(19) United States
(12) Patent Application Publication
(10) Pub. No.: US 2018/0090687 A1
(43) Pub. Date: Mar. 29, 2018

(54) CROSS-CONJUGATED POLYMER, MATERIAL FOR ELECTRONIC ELEMENT, MATERIAL FOR ORGANIC ELECTROLUMINESCENT ELEMENT, AND ORGANIC ELECTROLUMINESCENT ELEMENT

(71) Applicant: IDEMITSU KOSAN CO., LTD., Chiyoda-ku (JP)

(72) Inventors: Shigeru MATSUO, Ichihara-shi (JP); Hironori KAWAKAMI, Katsushika-ku (JP); Yuichiro KAWAMURA, Chiba-shi (JP); Masakazu FUNAHASHI, Chiba-shi (JP); Takahiro FUJIYAMA, Kisarazu-shi (JP)

(73) Assignee: IDEMITSU KOSAN CO., LTD, Chiyoda-ku (JP)

(21) Appl. No.: 15/561,743

(22) PCT Filed: Mar. 31, 2016
(86) PCT No.: PCT/JP2016/060783

§ 371 Date: Sep. 26, 2017

(30) Foreign Application Priority Data
Mar. 31, 2015 (JP) 2015-074537

(51) Int. Cl. H01L 51/00; H01L 51/50; C08G 61/10

(52) U.S. Cl. CPC H01L 51/0071; C07D 209/82; C08G 61/10; H01L 51/50

(57) ABSTRACT

A cross-conjugated polymer having a hole transporting property and a solubility suitable for forming a film by coating, a material including the polymer for electronic device and organic electroluminescence device, a solution including the polymer, and an organic electroluminescence device are described. The organic electroluminescence device contains a cathode, an anode, and at least one organic thin film layer disposed between the cathode and the anode and contains a light emitting layer. At least one of the at least one organic thin film layer is a hole injecting layer or a hole transporting layer, and at least one of the hole injecting layer and the hole transporting layer contains the cross-conjugated polymer having a structural unit (A) for forming a cross-conjugation system as a repeating unit and a substituent X containing at least one of an arylamine portion, a carbazole portion, and an indole portion as a side chain.

### TECHNICAL FIELD

The present invention relates to cross-conjugated polymers, materials for electronic devices comprising the polymers, materials for organic electroluminescence devices (also referred to as “organic EL device”) comprising the polymers, organic EL devices, coating solutions, methods of producing organic EL devices.

### BACKGROUND ART

In the production of an electronic device including an organic EL device, the film comprising a material for functioning the device has been generally formed by a vapor deposition method. On the other hand, the method of forming the film by a coating method has been also studied. However, a material sufficiently suitable for a coating solution still remains not obtained, and the research for developing a polymer material having both a charge transporting property and a solubility has been continued.

A polyvinylcarbazole (also referred to as “PVK”) has been long known as a material for organic EL device (Patent Literature 1, page 2, upper right hand column), and PVK has been further improved with the progress of the improvement in organic EL devices. Patent Literature 2 describes an organic EL device comprising a copolymer of a vinylanthracene derivative and a vinylcarbazole derivative, but a product with a high molecular weight has been not obtained.

Patent Literature 3 describes a copolymer of a unit having a carbazole derivative and a unit having an amino group, but a product with a high molecular weight has been not obtained.

### CITATION LIST

### Patent Literature

### Patent Literature 1: WO 2007/133632

### Patent Literature 2: JP 2010-196040A

### Patent Literature 2: JP 2005-309898A

### SUMMARY OF INVENTION

### Technical Problem

The present invention provides a cross-conjugated polymer suitable for use in forming a film by a coating method, a material for electronic device and a material for organic EL device each comprising the polymer, a solution comprising the polymer, and an organic EL device.

### Solution to Problem

As a result of extensive research for achieving the above object, the inventors have found that a cross-conjugated polymer comprising a structural unit (A) for forming a cross-conjugation system as a repeating unit and a specific substituent X as a side chain achieves the above object. The present invention is based on this finding.

(1) An organic electroluminescence device comprising a cathode, an anode, and at least one organic thin film layer which is disposed between the cathode and the anode and comprises a light emitting layer, wherein:

at least one layer of the at least one organic thin film layer is a hole injecting layer or a hole transporting layer; and

at least one of the hole injecting layer and the hole transporting layer comprises a cross-conjugated polymer comprising a structural unit (A) for forming a cross-conjugation system as a repeating unit and a substituent X comprising at least one selected from an arylamine portion, a carbazole portion, and an indole portion as a side chain.

(2) The organic electroluminescence device of (1), wherein the structural unit (A) is represented by at least one selected from formulae (A1) to (A5):

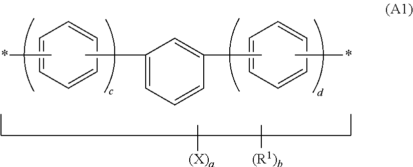

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1;

b is an integer of 0 to 3; and

each of c and d is independently an integer of 0 to 3;

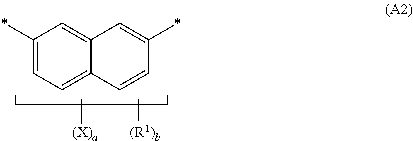

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1; and

b is an integer of 0 to 3

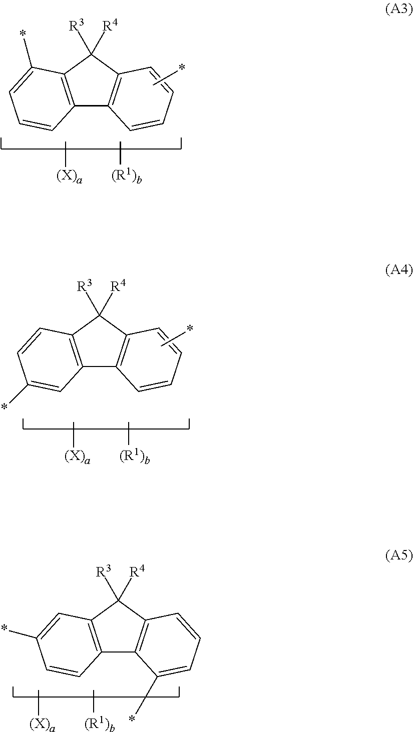

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an aralkyl group, or a substituted amino group;

each of R³ and R⁴ is independently a hydrogen atom, an alkyl group having 1 to 60 carbon atoms, an aryl group, a mono valent aromatic heterocyclic group, an aralkyl group, or a substituted amino group;

* is a bonding site in a main skeleton;

a is 0 or 1; and

b is an integer of 0 to 3.

(3) The organic electroluminescence device of (1) or (2), wherein the substituent X is represented by formula (HTG1):

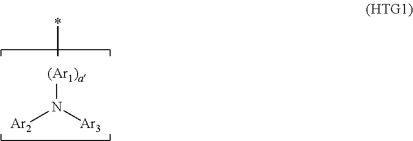

wherein:

* is a bonding site;

each of Ar₁, Ar₂, and Ar₃ is independently a substituted or unsubstituted aromatic hydrocarbon group having 6 to 60 ring carbon atoms, a substituted or unsubstituted aromatic heterocyclic group having 5 to 60 ring atoms, or a substituted or unsubstituted arylamino group;

Ar₂ and Ar₃ may be bonded to each other to form a ring;

a′ is 0 or 1;

when a′ is 0, the bonding site * exists on the nitrogen atom; and

when a′ is 1, the bonding site * exists on Ar₁, Ar₂, or Ar₃.

(4) The organic electroluminescence device of any one of (1) to (3), wherein the substituent X is bonded to the structural unit (A).
(5) The organic electroluminescence device of any one of (1) to (4), wherein the cross-conjugated polymer further comprises a structural unit (B) comprising at least one selected from an aromatic hydrocarbon ring group and an aromatic heterocyclic group as a repeating unit.
(6) The organic electroluminescence device of (5), wherein the structural unit (B) is represented by at least one selected from formula (B1) to (B3):

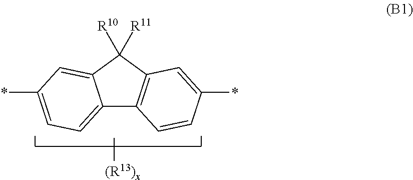

wherein:

each of R¹⁰ and R¹¹ is independently a hydrogen atom, an alkyl group having 1 to 60 carbon atoms, an aryl group, a mono valent aromatic heterocyclic group, an aralkyl group, or a substituted amino group;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

x is an integer of 0 to 3;

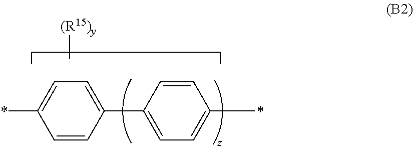

wherein:

each R¹⁵ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

y is an integer of 0 to 3; and

z is 0 or 1;

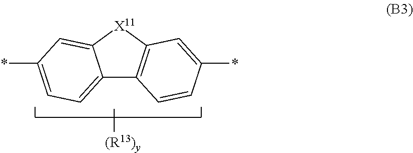

wherein:

X¹¹ is an oxygen atom or a sulfur atom;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

y is an integer of 0 to 3.

(7) The organic electroluminescence device of (5) or (6), wherein a molar ratio (M_{A}) of the structural unit (A) and a molar ratio (M_{B}) of the structural unit (B) are in a relationship represented by formula (1);

M_{A}≥M_{R} (1).

(8) The organic electroluminescence device of (7), wherein the molar ratio (M_{A}) of the structural unit (A) and the molar ratio (M_{B}) of the structural unit (B) are in a relationship represented by formula (2):

M_{A}>M_{B} (2).

(9) The organic electroluminescence device of any one of (1) to (8), wherein the cross-conjugated polymer further comprises a structural unit (C) comprising a cross-linkable group as a repeating unit.
(10) The organic electroluminescence device of (9), wherein the structural unit (C) is represented by at least one selected from formulae (C1) and (C2);

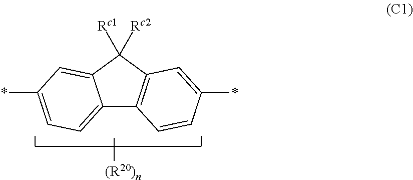

wherein:

each of R^{c1} and R^{c2} is independently a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

n is an integer of 0 to 3;

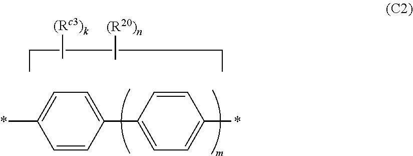

wherein:

each R^{c3} is a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

m is 0 or 1;

n is an integer of 0 to 3; and

k is an integer of 1 to 3.

(11) The organic electroluminescence device of any one of (1) to (10), wherein a weight average molecular weight of the cross-conjugated polymer is 50,000 to 1,500,000.
(12) A cross-conjugated polymer comprising a structural unit (A) and a structural unit (B) as repeating units and a substituent X as a side chain, wherein:

the structural unit (A) forms a cross-conjugation system;

the structural unit (B) comprises at least one selected from an aromatic hydrocarbon ring group and an aromatic heterocyclic group in a main chain;

the substituent X comprises at least one selected from an arylamine portion, a carbazole portion, and an indole portion; and

a molar ratio (M_{A}) of the structural unit (A) and a molar ratio (M_{B}) of the structural unit (B) are in a relationship represented by formula (1):

M_{A}≥M_{B} (1).

(13) The cross-conjugated polymer of (12), wherein the molar ratio (M_{A}) of the structural unit (A) and the molar ratio (M_{B}) of the structural unit (B) are in a relationship represented by formula (2):

M_{A}>M_{B} (2).

(14) The cross-conjugated polymer of (12) or (13), wherein the structural unit (A) is represented by at least one selected from formulae (A1) to (A5):

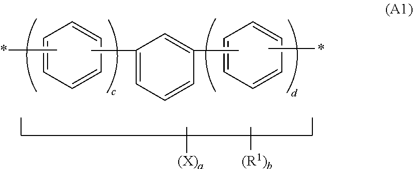

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1;

b is an integer of 0 to 3; and

each of c and d is independently an integer of 0 to 3;

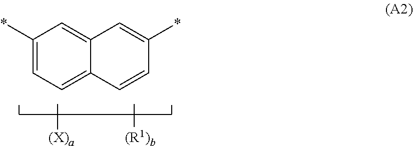

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1; and

b is an integer of 0 to 3;

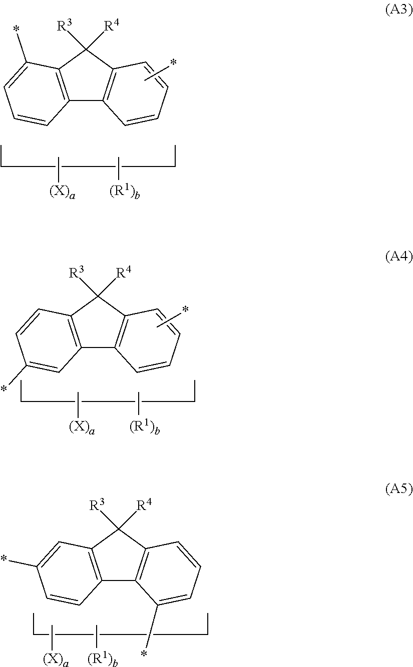

wherein:

X is the substituent X comprising at least one selected from the arylamine portion, the carbazole portion, and the indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

each of R³ and R⁴ is independently a hydrogen atom, an alkyl group having 1 to 60 carbon atoms, an aryl group, a mono valent aromatic heterocyclic group, an aralkyl group, or a substituted amino group;

* is a bonding site in a main skeleton;

a is 0 or 1; and

b is an integer of 0 to 3.

(15) The cross-conjugated polymer of any one of (12) to (14), wherein the substituent X is represented by formula (HTG1):

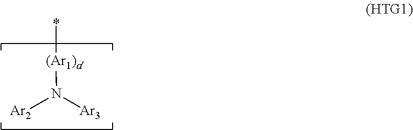

wherein:

* is a bonding site;

each of Ar₁, Ar₂, and Ara is independently a substituted or unsubstituted aromatic hydrocarbon group having 6 to 60 ring carbon atoms, a substituted or unsubstituted aromatic heterocyclic group having 5 to 60 ring atoms, or a substituted or unsubstituted arylamino group;

Ar₂ and Ara may be bonded to each other to form a ring;

a′ is 0 or 1;

when a′ is 0, the bonding site * exists on the nitrogen atom; and

when a′ is 1, the bonding site * exists on Ar₁, Ar₂, or Ara.

(16) The cross-conjugated polymer of any one of (12) to (15), wherein the substituent X is bonded to the structural unit (A).
(17) The cross-conjugated polymer of any one of (12) to (16), wherein the structural unit (B) is represented by at least one selected from formula (B1) to (B3):

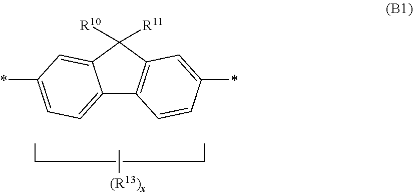

wherein:

each of R¹⁰ and R¹¹ is independently a hydrogen atom, an alkyl group having 1 to 60 carbon atoms, an aryl group, a mono valent aromatic heterocyclic group, an aralkyl group, or a substituted amino group;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main chain; and

x is an integer of 0 to 3;

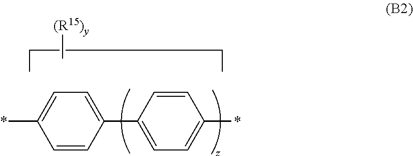

wherein:

each R¹⁵ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

y is an integer of 0 to 3; and

z is 0 or 1;

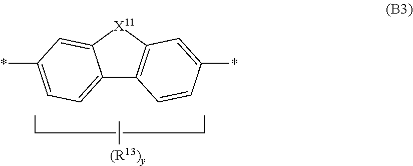

wherein:

X¹¹ is an oxygen atom or a sulfur atom;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

y is an integer of 0 to 3.

(18) The cross-conjugated polymer of any one of (12) to (17), wherein the cross-conjugated polymer further comprises a structural unit (C) comprising a cross-linkable group as a repeating unit.
(19) The cross-conjugated polymer of (18), wherein the structural unit (C) is represented by at least one selected from formulae (C1) and (C2):

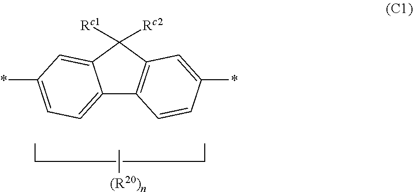

wherein:

each of R^{c1} and R^{c2} is independently a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

n is an integer of 0 to 3;

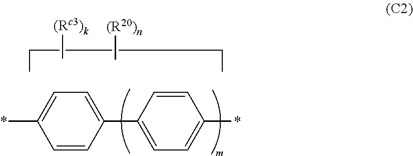

wherein:

each R^{c3} is a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

m is 0 or 1;

n is an integer of 0 to 3; and

k is an integer of 1 to 3.

(20) The cross-conjugated polymer of (18) or (19), wherein a molar ratio (M_{A}) of the structural unit (A), a molar ratio (M_{B}) of the structural unit (B), and a molar ratio (M_{C}) of the structural unit (C) are in a relationship represented by formula (3)

M_{A}≥M_{B}+M_{C} (3).

(21) The cross-conjugated polymer of any one of (12) to (20), wherein a weight average molecular weight of the cross-conjugated polymer is 50,000 to 1,500,000.
(22) A material for electronic device comprising the polymer of any one of (12) to (21).
(23) A material for organic electroluminescence device comprising the polymer of any one of (12) to (21).
(24) An organic electroluminescence device comprising an organic thin film layer between an anode and a cathode, wherein:

the organic thin film layer comprises one or more layers which comprise a light emitting layer; and

at least one layer of the organic thin film layer comprises the polymer of any one of (12) to (21).

(25) The organic electroluminescence device of (24), wherein the organic thin film layer comprises a hole transporting layer, and the hole transporting layer comprises the polymer.
(26) A coating solution comprising the polymer of any one of (12) to (21) and a solvent.
(27) A method of producing an organic electroluminescence device, which comprises forming a thin film layer by using the coating solution of (26).
(28) The method of (27), wherein the thin film layer is formed by a wet film-forming method.

### Advantageous Effects of Invention

According to the present invention, a cross-conjugated polymer suitable for use in the formation of a film by a coating method, a material for electronic device or organic EL device comprising the polymer, a solution comprising the polymer, and an organic EL device are provided.

### DESCRIPTION OF EMBODIMENTS

The organic electroluminescence device of the invention comprises a cathode, an anode, and at least one organic thin film layer which is disposed between the cathode and the anode and comprises a light emitting layer, wherein:

the at least one layer of the at least one organic thin film layer is a hole injecting layer or a hole transporting layer;

at least one of the hole injecting layer and the hole transporting layer comprises a cross-conjugated polymer comprising a structural unit (A) for forming a cross-conjugation system as a repeating unit and a substituent X comprising at least one selected from an arylamine portion, a carbazole portion, and an indole portion as a side chain.

The definition of “hydrogen atom” used herein includes isotopes different in the neutron numbers, i.e., light hydrogen (protium), heavy hydrogen (deuterium), and tritium. This definition equally applies to hydrogen atoms of the copolymer in an aspect of the present invention.

The term of “a to b carbon atoms” referred to by “a substituted or unsubstituted group XX having a to b carbon atoms” or similar expression used herein is the number not including any carbon atom in the substituent of the substituted group XX.

Examples of the optional substituent referred to by “substituted or unsubstituted” used herein include an alkyl group having 1 to 50, preferably 1 to 10, more preferably 1 to 5 carbon atoms; a cycloalkyl group having 3 to 50, preferably 3 to 6, more preferably 5 or 6 ring carbon atoms; an aryl group having 6 to 50, preferably 6 to 24, more preferably 6 to 12 ring carbon atoms; an aralkyl group composed of an aryl group having 6 to 50, preferably 6 to 24, more preferably 6 to 12 ring carbon atoms and an alkyl portion having 1 to 50, preferably 1 to 10, more preferably 1 to 5 carbon atoms; an amino group; a mono- or dialkylamino group having an alkyl group having 1 to 50, preferably 1 to 10, more preferably 1 to 5 carbon atoms; a mono- or diarylamino group having an aryl group having 6 to 50, preferably 6 to 24, more preferably 6 to 12 ring carbon atoms; an alkoxy group having an alkyl group having 1 to 50, preferably 1 to 10, more preferably 1 to 5 carbon atoms; an aryloxy group having an aryl group having 6 to 50, preferably 6 to 24, more preferably 6 to 12 ring carbon atoms; a mono-, di- or tri-substituted silyl group having a group selected from an alkyl group having 1 to 50, preferably 1 to 10, more preferably 1 to 5 carbon atoms and an aryl group having 6 to 50, preferably 6 to 24, more preferably 6 to 12 ring carbon atoms; a heterocyclic group having 5 to 50, preferably 5 to 24, more preferably 5 to 12 ring atoms and 1 to 5, preferably 1 to 3, more preferably 1 to 2 hetero atoms, such as a nitrogen atom, an oxygen atom and a sulfur atom; a haloalkyl group having 1 to 50, preferably 1 to 10, more preferably 1 to 5 carbon atoms and 1 to 8, preferably 1 to 5, more preferably 1 to 3 halogen atoms, such as a fluorine atom, a chlorine atom, a bromine atom and an iodine atom; a halogen atom, such as a fluorine atom, a chlorine atom, a bromine atom and an iodine atom; a cyano group; and a nitro group.

The optional group is preferably selected from a halogen atom, a cyano group, a trialkylsilyl group having an alkyl group having 1 to 5 carbon atoms, an alkyl group having 1 to 5 carbon atoms, a cycloalkyl group having 5 or 6 ring carbon atoms, and an aryl group having 6 to 12 ring carbon atoms.

### Cross-Conjugated Polymer

The cross-conjugated polymer comprises a structural unit (A) for forming a cross-conjugation system as a repeating unit and a substituent X comprising at least one selected from an arylamine portion, a carbazole portion, and an indole portion as a side chain. The substituent X is preferably included in the structural unit (A).

### Structural Unit (A)

The structural unit (A) forms a cross-conjugation system.

The cross-conjugation system is a conjugation, wherein in a set of three 7T bonds only two π bonds interact with each other by conjugation and the third π bond is excluded from the interaction. The 7T conjugation in the polymer main chain is discontinued by the presence of the structural unit (A) for forming a cross-conjugation system. Therefore, the energy difference between HOMO and LUMO (band gap) is not made extremely small even when introducing a conjugated structural unit into the polymer main chain, thereby reducing the adverse effect attributable to the polymer main chain on the emission properties.

The structural unit (A) is preferably represented by at least one selected from formulae (A1) to (A5) and more preferably represented by formula (A1):

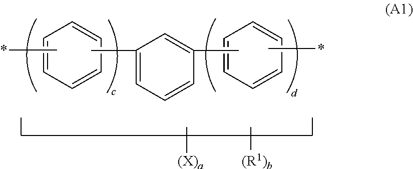

wherein:

X is the substituent X comprising at least one selected from an arylamine portion, a carbazole portion, and an indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1;

b is an integer of 0 to 3; and

each of c and d is independently an integer of 0 to 3;

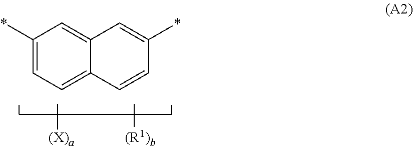

wherein:

X is the substituent X comprising at least one selected from an arylamine portion, a carbazole portion, and an indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

a is 0 or 1; and

b is an integer of 0 to 3

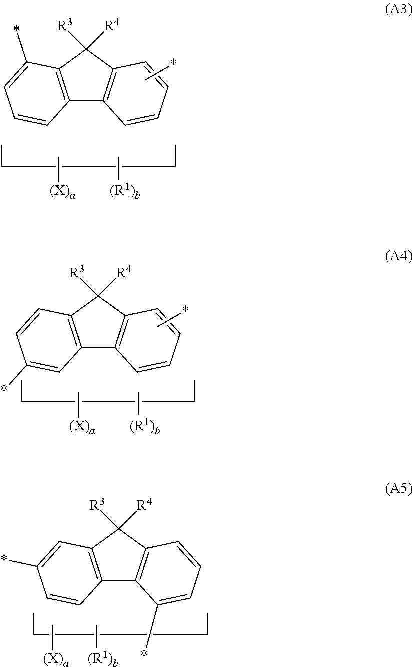

wherein:

X is the substituent X comprising at least one selected from an arylamine portion, a carbazole portion, and an indole portion;

each R¹ is independently a tertiary alkyl group having 4 to 60 carbon atoms, an aryl group, a mono valent aromatic heterocyclic group, an aralkyl group, or a substituted amino group;

each of R³ and R⁴ is independently a hydrogen atom, an alkyl group having 1 to 60 carbon atoms, an aryl group, a mono valent aromatic heterocyclic group, an aralkyl group, or a substituted amino group;

* is a bonding site in a main skeleton;

a is 0 or 1; and

b is an integer of 0 to 3.

The structural unit (A) is more preferably represented by formula (A1-1):

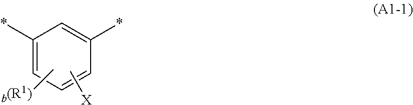

wherein:

X is the substituent X comprising at least one selected from an arylamine portion, a carbazole portion, and an indole portion;

each R¹ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

b is an integer of 0 to 3; and

X is preferably bonded to 5-position when * of the phenylene portion is taken as 1-position.

Each variable of formulae (A1) to (A5) and (A1-1) is described below. The subscript a is preferably 1.

The subscript b is preferably an integer of 0 to 2, more preferably 0 or 1, and further more preferably 0.

R¹ is preferably a tertiary alkyl group having 4 to 60 carbon atoms, an aryl group, or a mono valent aromatic heterocyclic group.

Each of R³ and R⁴ is preferably a hydrogen atom, an alkyl group having 1 to 60 carbon atoms, an aryl group, a mono valent aromatic heterocyclic group, an aralkyl group, or a substituted amino group, more preferably an alkyl group having 1 to 60 carbon atoms, and further more preferably a methyl group.

The tertiary alkyl group for R¹ has preferably 4 to 60, more preferably 4 to 30, further more preferably 4 to 20, and still further more preferably 4 to 10 carbon atoms.

Examples of the tertiary alkyl group having 4 to 60 carbon atoms for R¹ include a t-butyl group, a t-pentyl group, a 1,1-dimethylbutyl group, a 1,1-dimethylpentyl group, a 1,1-dimethylhexyl group, a 1,1-dimethylheptyl group, a 1,1-diethylhexyl group, a 1,1-dimethyldecyl group, and a1,1-dimethylundecyl group.

The alkyl group for R³ and R⁴ has preferably 1 to 60, more preferably 1 to 30, further more preferably 1 to 20, and still further more preferably 1 to 10 carbon atoms.

Examples of the alkyl group having 1 to 60 carbon atoms for R³ and R⁴ include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups).

The aryl group for R¹, R³, and R⁴ has preferably 6 to 60, more preferably 6 to 30, further more preferably 6 to 20 carbon atoms.

Examples of the aryl group for R¹ include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a biphenyl-2-yl group, a biphenyl-3-yl group, a biphenyl-4-yl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenyl-4-yl group, a 4″-t-butyl-p-terphenyl-4-yl group, a fluoren-1-yl group, a fluoren-2-yl group, a fluoren-3-yl group, and a fluoren-4-yl group. Preferred are a phenyl group, a naphthyl group, an anthryl group, a biphenyl group, a p-terphenyl group, a tolyl group, a fluorenyl group, a phenylene group, a naphthylene group, an anthracenylene group, a biphenylene group, a p-terphenylene group, a tolylene group, and a fluorenylene group.

The mono valent aromatic heterocyclic group for R¹, R³, and R⁴ has preferably 5 to 60, more preferably 5 to 30, and further more preferably 5 to 15 ring atoms.

Examples of the mono valent aromatic heterocyclic group for R¹, R³, and R⁴ include a 1-pyrrolyl group, a 2-pyrrolyl group, a 3-pyrrolyl group, a pyrazinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 2-dibenzofuranyl group, a 4-dibenzofuranyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-phenanthridinyl group, 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1,7-phenanthroline-2-yl group, a 1,7-phenanthroline-3-yl group, a 1,7-phenanthroline-4-yl group, a 1,7-phenanthroline-5-yl group, a 1,7-phenanthroline-6-yl group, a 1,7-phenanthroline-8-yl group, a 1,7-phenanthroline-9-yl group, a 1,7-phenanthroline-10-yl group, a 1,8-phenanthroline-2-yl group, a 1,8-phenanthroline-3-yl group, a 1,8-phenanthroline-4-yl group, a 1,8-phenanthroline-5-yl group, a 1,8-phenanthroline-6-yl group, a 1,8-phenanthroline-7-yl group, a 1,8-phenanthroline-9-yl group, a 1,8-phenanthroline-10-yl group, a 1,9-phenanthroline-2-yl group, a 1,9-phenanthroline-3-yl group, a 1,9-phenanthroline-4-yl group, a 1,9-phenanthroline-5-yl group, a 1,9-phenanthroline-6-yl group, a 1,9-phenanthroline-7-yl group, a 1,9-phenanthroline-8-yl group, a 1,9-phenanthroline-10-yl group, a 1,10-phenanthroline-2-yl group, a 1,10-phenanthroline-3-yl group, a 1,10-phenanthroline-4-yl group, a 1,10-phenanthroline-5-yl group, a 2,9-phenanthroline-1-yl group, a 2,9-phenanthroline-3-yl group, a 2,9-phenanthroline-4-yl group, a 2,9-phenanthroline-5-yl group, a 2,9-phenanthroline-6-yl group, a 2,9-phenanthroline-7-yl group, a 2,9-phenanthroline-8-yl group, a 2,9-phenanthroline-10-yl group, a 2,8-phenanthroline-1-yl group, a 2,8-phenanthroline-3-yl group, a 2,8-phenanthroline-4-yl group, a 2,8-phenanthroline-5-yl group, a 2,8-phenanthroline-6-yl group, a 2,8-phenanthroline-7-yl group, a 2,8-phenanthroline-9-yl group, a 2,8-phenanthroline-10-yl group, a 2,7-phenanthroline-1-yl group, a 2,7-phenanthroline-3-yl group, a 2,7-phenanthroline-4-yl group, a 2,7-phenanthroline-5-yl group, a 2,7-phenanthroline-6-yl group, a 2,7-phenanthroline-8-yl group, a 2,7-phenanthroline-9-yl group, a 2,7-phenanthroline-10-yl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrole-1-yl group, a 2-methylpyrrole-3-yl group, a 2-methylpyrrole-4-yl group, a 2-methylpyrrole-5-yl group, a 3-methylpyrrole-1-yl group, a 3-methylpyrrole-2-yl group, a 3-methylpyrrole-4-yl group, a 3-methylpyrrole-5-yl group, a 2-t-butylpyrrole-4-yl group, a 3-(2-phenylpropyl)pyrrole-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a 2-thienyl group, a 3-thienyl group, a 2-benzothiophenyl group, a 3-thiophenyl group, a 4-thiophenyl group, a 5-thiophenyl group, a 6-thiophenyl group, a 7-thiophenyl group, a 1-isothiophenyl group, a 3-isothiophenyl group, a 4-isothiophenyl group, a 5-isothiophenyl group, a 6-isothiophenyl group, a 7-thiophenyl group, a 2-dibenzothiophenyl group, and a 4-dibenzothiophenyl group.

The alkoxy group having 1 to 20 carbon atoms for R¹ is represented, for example, by —OY, wherein Y is selected from the alkyl group mentioned above. Examples of the alkoxy group include a methoxy group and an ethoxy group.

The cycloalkoxy group having 3 to 10 carbon atoms for R¹ is represented, for example, by —OY, wherein Y is selected from the cycloalkyl group mentioned above. Examples of the cycloalkoxy group include a cyclopentyloxy group and a cyclohexyloxy group.

The aryloxy group having 6 to 30 carbon atoms for R¹ is represented, for example, by —OY, wherein Y is selected from the aromatic hydrocarbon ring mentioned above. Example of the aryloxy group includes a phenoxy group.

The aralkyl group for R¹ is represented, for example, by —Y—Z, wherein Y is an alkylene group corresponding to the alkyl group mentioned above and Z is selected from the aryl group mentioned above. The aryl portion has preferably 6 to 30, more preferably 6 to 15 ring carbon atoms, and the alkyl portion has preferably 1 to 10, more preferably 1 to 6 carbon atoms. Examples thereof include a benzyl group, a phenylethyl group, a 2-phenylpropane-2-yl group.

The substituted amino group for R¹ is represented, for example, by —NY₂, wherein Y is selected from the alkyl group and the aryl group each mentioned above.

The alkylamino group is a monoalkyl amino group or a dialkylamino group, preferably a dialkylamino group. The alkyl portion is selected from the alkyl group mentioned above. The alkyl portion of the mono- or dialkylamino group has preferably 1 to 20 carbon atoms.

The arylamino group is a monoarylamino group, a diarylamino group, or an alkylarylamino group, preferably a diarylamino group or an alkylarylamino group. The aryl portion of the mono- or diarylamino group has preferably 6 to 30, more preferably 6 to 15 ring carbon atoms. The aryl group to be bonded to the nitrogen atom is selected from the aryl group mentioned above.

The substituted silyl group for R¹ has at least one substituent selected from an alkyl group and an aryl group or at least one substituent selected from an alkyloxy group and an aryloxy group.

In the substituted silyl group having at least one substituent selected from an alkyl group and an aryl group, the alkyl group and the aryl group to be bonded to the silicon atom are selected from the alkyl group and the aryl group each mentioned above. The alkyl substituents on the silyl group may be the same or different. Also, the aryl substituents on the silyl group may be the same or different. The substituents on the silyl group may be mixed substituents of an aryl group and an alkyl group. In view of improving the solubility, preferred are a trialkylsilyl group, a triarylsilyl group, a dialkylarylsilyl group, and an alkyldiarylsilyl group. Examples of the trialkylsilyl group include a trimethylsilyl group, a triethylsilyl group, an ethyldimethylsilyl group, a tripropylsilyl group, a propyldimethylsilyl group, a tributylsilyl group, a t-butyldimethylsilyl group, a tripentylsilyl group, a triheptylsilyl group, and a trihexylsilyl group, with a trimethylsilyl group, a triethylsilyl group, and an ethyldimethylsilyl group being preferred.

Examples of the triarylsilyl group include a triphenylsilyl group, a trinaphthylsilyl group, and a diphenylbenzylsilyl group, with a triphenylsilyl group being preferred. Examples of the dialkylarylsilyl group and the alkyldiarylsilyl group include a dimethylphenylsilyl group, a diethylphenylsilyl group, a diphenylmethylsilyl group, and an ethyldiphenylsilyl group, with a diphenylmethylsilyl group and an ethyldiphenylsilyl group being preferred.

In the substituted silyl group having at least one substituent selected from an alkyloxy group and aryloxy group, the alkyl group and the aryl group bonded to the oxygen atom are selected from the aryl group and the alkyl group each mentioned above. The alkyloxy substituents on the silyl group may be the same or different. The aryloxy substituents on the silyl group may be the same or different. The substituents on the silyl group may be mixed substituents of an aryloxy group and an alkyloxy group. In view of improving the solubility, preferred are a trialkyloxysilyl group and a triaryloxysilyl group. Examples of the trialkyloxysilyl group include a trimethoxysilyl group, a triethoxysilyl group, an ethoxydimethoxysilyl group, a tripropoxysilyl group, a propyloxydimethoxysilyl group, and tributoxysilyl group, with a trimethoxysilyl group and a triethoxysilyl group being preferred.

Examples of the triaryloxysilyl group include a triphenyloxysilyl group, a trinaphthyloxysilyl group, and a diphenyloxybenzyloxysilyl group.

The halogen atom for R¹ include a fluorine atom, a chlorine atom, a bromine atom, and an iodine atom, with a fluorine atom being particularly preferred.

Examples of the structural unit (A) are shown below, although not limited thereto. X is the substituent X.

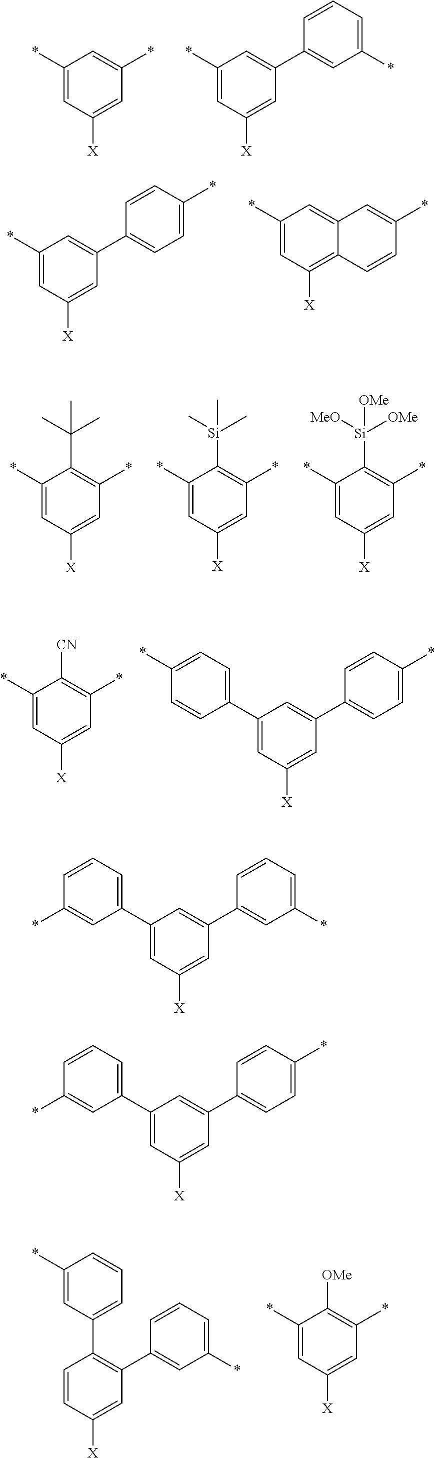

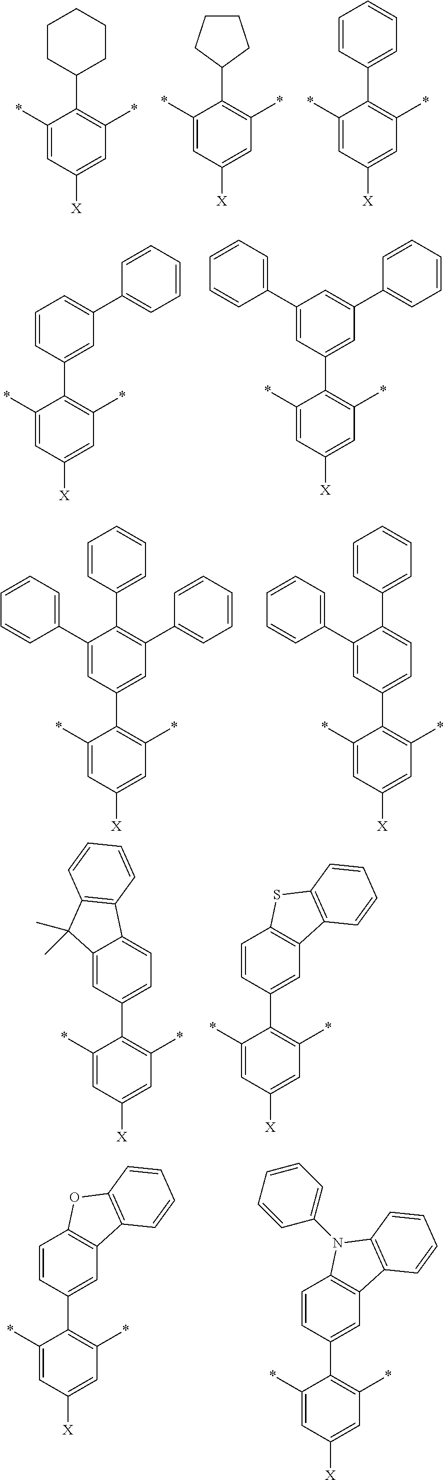

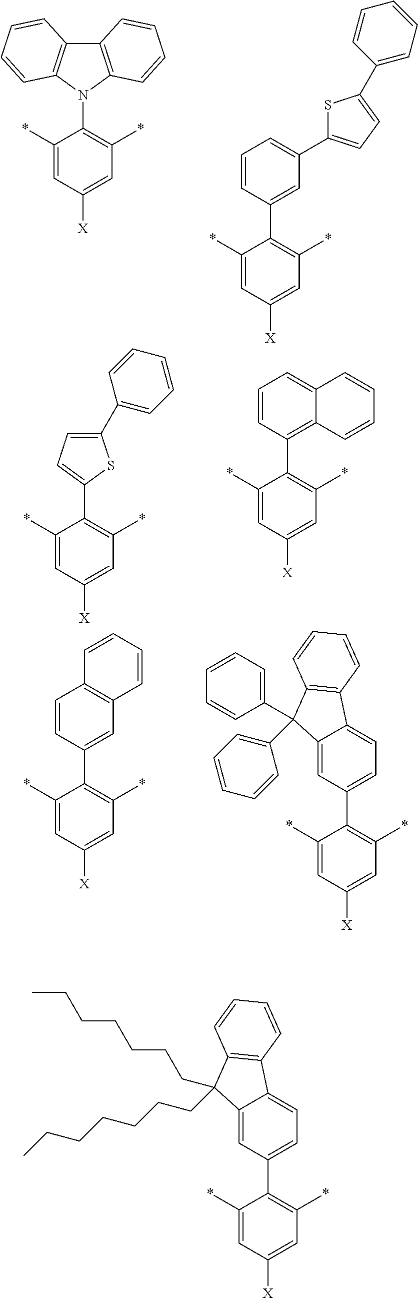

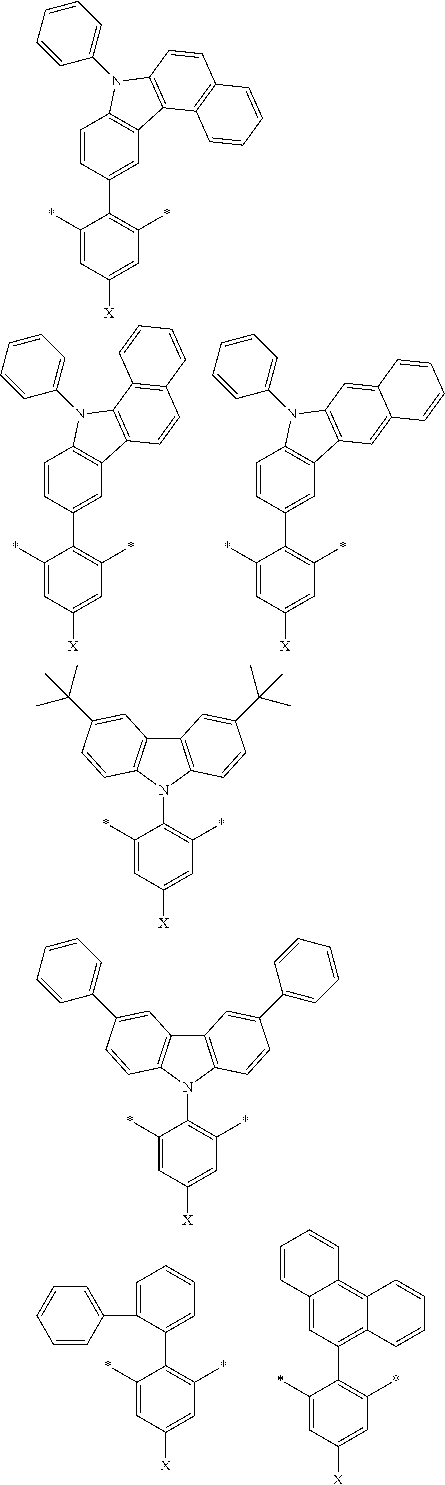

wherein X is the substituent X comprising at least one selected from an arylamine portion, a carbazole portion, and an indole portion. The substituent X is preferably represented by formula (HTG1), more preferably represented by at least one selected from formulae (HTG2a), (HTG2b), (HTG2c), (HTG2d), and (HTG2e), and further more preferably represented by at least one selected from formulae (HTG2a), (HTG2b), and (HTG2c).

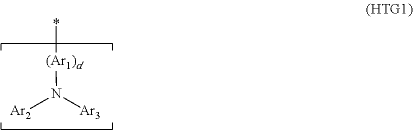

wherein:

* is a bonding site;

each of Ar₁, Ar₂, and Ar₃ is independently a substituted or unsubstituted aromatic hydrocarbon group having 6 to 60 ring carbon atoms, a substituted or unsubstituted aromatic heterocyclic group having 5 to 60 ring atoms, or a substituted or unsubstituted arylamino group;

Ar₂ and Ar₃ may be bonded to each other to form a ring;

a′ is 0 or 1;

when a′ is 0, the bonding site * exists on the nitrogen atom; and

when a′ is 1, the bonding site * exists on Ar₁, Ar₂, or Ar₃;

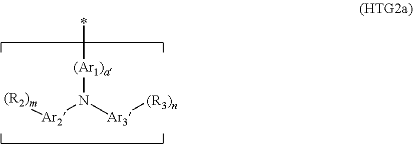

wherein:

Ar₁ is the same as Ar₁ of formula (HTG1);

each of Ar₂′ and Ar₃′ is independently a substituted or unsubstituted aromatic hydrocarbon group having 6 to 60 ring carbon atoms;

each of R₂ and R₃ is independently a substituted or unsubstituted aromatic hydrocarbon group having 6 to 60 ring carbon atoms or a substituted or unsubstituted aromatic heterocyclic group having 5 to 60 ring atoms;

R₂ and R₃ may be bonded to each other to form a ring;

Ar₂′ and Ar₃′ may be bonded to each other to form a ring;

a′ and a bonding site * are as defined in formula (HTG1), wherein the bonding site * may exist on any of positions inclusive of R₂, R₃, Ar₂′, and Ar₃′; and

each of m and n is independently an integer of 0 to 5;

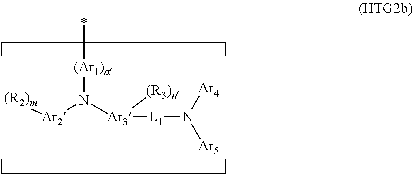

wherein:

each of Ar₁, Ar₄, and Ar₅ is the same as Ar₁ of formula (HTG1);

each of Ar₂′ and Ar₃′ is the same as Ar₂′ of formula (HTG2a);

each of R₂ and R₃ is the same as R₂ of formula (HTG2a);

L₁ is a single bond, a substituted or unsubstituted aromatic hydrocarbon group having 6 to 60 ring carbon atoms, or a substituted or unsubstituted aromatic heterocyclic group having 5 to 60 ring atoms;

R₂ and R₃ may be bonded to each other to form a ring;

Ar₂′ and Ar₃′ may be bonded to each other to form a ring;

Ar₄ and Ar₅ may be bonded to each other to form a ring;

a′ and a bonding site * are as defined in formula (HTG1), wherein the bonding site * may exist on any of positions inclusive of R₂, R₃, L₁, Ar₄, Ar₅, Ar₂′, and Ar₃′;

m is the same as m of formula (HTG2a); and

n′ is an integer of 0 to 4;

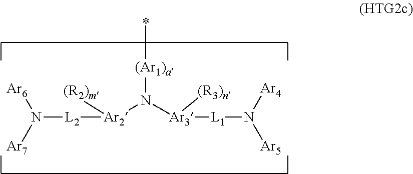

wherein:

each of Ar₁, Ar₄, Ar₅, Ar₆, and Ar₇ is the same as Ar₁ of formula (HTG1);

each of Ar₂′ and Ar₃′ is the same as Ar₂′ of formula (HTG2a);

each of R₂ and R₃ is the same as R₂ of formula (HTG2a);

each of L₁ and L₂ is the same as L₁ of formula (HTG2b);

R₂ and R₃ may be bonded to each other to form a ring;

Ar₂′ and Ar₃′ may be bonded to each other to form a ring;

Ar₄ and Ar₅ may be bonded to each other to form a ring;

Ar₆ and Ar₇ may be bonded to each other to form a ring;

a′ and a bonding site * are as defined in formula (HTG1), wherein the bonding site * may exist on any of positions inclusive of R², R₃, L₁, L₂, Ar₄, Ar₅, Ar₆, Ar₇, Ar₂′, and Ar₃′; and

m′ and n′ is the same as n′ of formula (HTG2b);

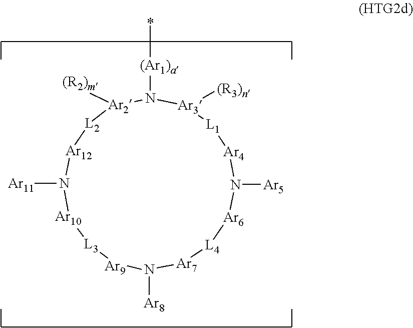

wherein;

each of Ar₁, Ar₄, Ar₅, Ar₆, Ar₇, Ar₈, Ar₉, Ar₁₀, Ar₁₁, and Ar₁₂ is the same as Ar₁ of formula (HTG1);

each of Ar₂′ and Ar₃′ is the same as Ar₂′ of formula (HTG2a);

each of R₂ and R₃ is the same as R₂ of formula (HTG2a);

each of L₁, L₂, L₃, and L₄ is the same as L₁ of formula (HTG2b);

R₂ and R₃ may be bonded to each other to form a ring;

Ar₂′ and Ar₃′ may be bonded to each other to form a ring;

Ar₄ and Ar₆ may be bonded to each other to form a ring;

Ar₇ and Ar₉ may be bonded to each other to form a ring;

Ar₁₀ and Ar₁₂ may be bonded to each other to form a ring;

a′ and a bonding site * are as defined in formula (HTG1), wherein the bonding site * may exist on any of positions inclusive of R₂, R₃, L₁, L₂, L₃, L₄, Ar₁, Ar₄, Ar₅, Ar₆, Ar₇, Ar₈, Ar₉, Ar₁₀, Ar₁₁, Ar₁₂, Ar₂′, and Ar₃′; and

each of m′ and n′ is the same as n′ of formula (HTG2b);

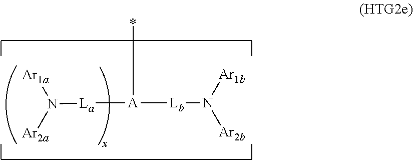

wherein:

each of Ar₁ₐ, Ar₂ₐ, Ar_{1b}, and Ar_{2b} is the same as Ar₁ of formula (HTG1);

A is a substituted or unsubstituted aromatic hydrocarbon group having 6 to 60 ring carbon atoms or a substituted or unsubstituted aromatic heterocyclic group having 5 to 60 ring atoms;

each of Lₐ and L_{b} is the same as L₁ of formula (HTG2b);

Ar₁ₐ and Ar₂ₐ may be bonded to each other to form a ring;

Ar_{1b} and Ar_{2b} may be bonded to each other to form a ring;

x is an integer of 1 or more; and

a bonding site * may exist on any position, i.e., the bonding site * may exist on any of positions inclusive of Lₐ, L_{b}, Ar₁ₐ, Ar₂ₐ, Ar_{1b}, and Ar_{2b}.

The substituent X is further more preferably represented by at least one selected from formulae (HTG3a), (HTG3b), and (HTG3c), more preferably represented by formula (HTG3a) or (HTG3b):

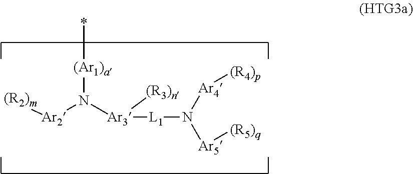

wherein:

Ar₁ is the same as Ar₁ of formula (HTG1);

each of Ar₂′ and Ar₃′ is the same as Ar₂′ of formula (HTG2a);

each of R₂, R₃, R₄, and R₅ is the same as R₂ of formula (HTG2a);

L₁ is the same as L₁ of formula (HTG2b);

R₂, R₃, R₄, and R₅ may be bonded to each other to form a ring;

Ar₂′ and Ar₃′ may be bonded to each other to form a ring;

Ar₄′ and Ar₅′ may be bonded to each other to form a ring;

a′ and a bonding site * are as defined in formula (HTG1), wherein the bonding site * may exist on any of positions inclusive of R₂, R₃, R₄, R₅, L₁, Ar₁, Ar₂′, Ar₃′, Ar₄′, and Ar₅′;

each of m, p, and q is the same as m of formula (HTG2a); and

n′ is the same as n′ of formula (HTG2b);

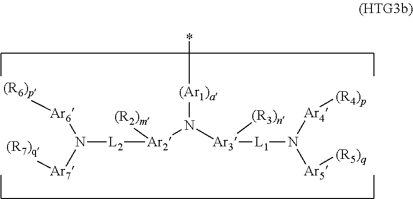

wherein:

Ar₁ is the same as Ar₁ of formula (HTG1);

each of Ar₂′, Ar₃′, Ar₄′, Ar₅′, Ar₆′, and Ar₇′ is the same as Ar₂′ of formula (HTG2a);

each of R₂, R₃, R₄, R₅, R₆, and R₇ is the same as R₂ of formula (HTG2a);

each of L₁ and L₂ is the same as L₁ of formula (HTG2b);

R₂, R₃, R₄, and R₅ may be bonded to each other to form a ring;

Ar₂′ and Ar₃′ may be bonded to each other to form a ring;

Ar₄′ and Ar₅′ may be bonded to each other to form a ring;

Ar₆′ and Ar₇′ may be bonded to each other to form a ring;

a′ and a bonding site * are as defined in formula (HTG1), wherein the bonding site * may exist on any of positions inclusive of R₂, R₃, R₄, R₅, R₆, R₇, L₁, L₂, Ar₁, Ar₂′, Ar₃′, Ar₄′, Ar₅′, Ar₆′, and Ar₇′;

each of p, q, p′, and q′ is the same as m of formula (HTG2a); and

each of m′ and n′ is the same as n′ of formula (HTG2b);

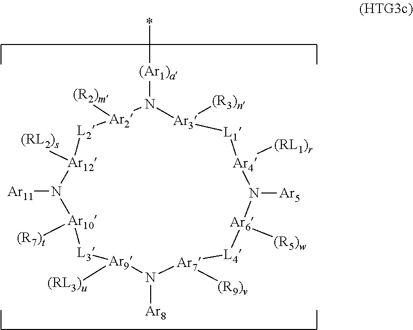

wherein:

each of Ar₁, Ar₅, Ar₈, and Ar₁₁ is the same as Ar₁ of formula (HTG1);

each of Ar₂′, Ar₃′, Ar₄′, Ar₆′, Ar₇′, Ar₉′, Ar₁₀′, and Ar₁₂′ is the same as Ar₂′ of formula (HTG2a);

each of R₂, R₃, R₅, R₇, R₉, RL₁, RL₂, and RL₃ is the same as R₂ of formula (HTG2a);

each of L₁′, L₂′, L₃′, and L₄′ is the same as L₁ of formula (HTG2b);

R₂, R₃, R₅, R₇, R₉, RL₁, RL₂, and RL₃ may be bonded to each other to form a ring;

Ar₂′ and Ar₃′ may be bonded to each other to form a ring;

Ar₄′ and Ar₆′ may be bonded to each other to form a ring;

Ar₇′ and Ar₉′ may be bonded to each other to form a ring;

Ar₁₀′ and Ar₁₂′ may be bonded to each other to form a ring;

a′ and a bonding site * are as defined in formula (HTG1), wherein the bonding site * may exist on any of positions inclusive of R₂, R₃, R₅, R₇, R₉, RL₁, RL₂, RL₃, L₁′, L₂′, L₃′, L₄′, Ar₁, Ar₅, Ar₈, Ar₉, Ar₂′, Ar₃′, Ar₄′, Ar₆′, Ar₇′, Ar₉′, Ar₁₀′, and Ar₁₂′; and

each of m′, n′, s, t, u, v, r, and w is the same as n′ of formula (HTG2b).

Examples of the substituted or unsubstituted aromatic hydrocarbon group having 6 to 60 ring carbon atoms for Ar₁, R₂, and L₁ include the aromatic hydrocarbon ring group mentioned below and a divalent residue thereof. The aromatic hydrocarbon ring group has preferably 6 to 60, more preferably 6 to 30, and further more preferably 6 to 15 ring carbon atoms.

Examples of the aromatic hydrocarbon ring group include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a biphenyl-2-yl group, a biphenyl-3-yl group, a biphenyl-4-yl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenyl-4-yl group, a 4″-t-butyl-p-terphenyl-4-yl group, a fluoren-1-yl group, a fluoren-2-yl group, a fluoren-3-yl group, and a fluoren-4-yl group. Preferred are a phenyl group, a naphthyl group, an anthryl group, a biphenyl group, a p-terphenyl group, a tolyl group, a fluorenyl group, a phenylene group, a naphthylene group, an anthracenylene group, a biphenylene group, a p-terphenylene group, a tolylene group, and a fluorenylene group.

Examples of the substituent of the aromatic hydrocarbon group include an alkyl group having 1 to 10 carbon atoms, a halogen atom, a heteroaryl group, such as a carbazolyl group, a dibenzofuranyl group, and a dibenzothiophenyl group, an aryl group, such as a 9,9-dioctylfluorenyl group, a diarylamino group, such as a diphenylamino group, and a cyano group.

Examples of the substituted or unsubstituted aromatic heterocyclic group having 5 to 60 ring atoms for Ar₁, R₂, and L₁ include the aromatic heterocyclic group mentioned below and a divalent residue thereof. The aromatic heterocyclic group has preferably 5 to 60, more preferably 5 to 30, and further more preferably 5 to 15 ring atoms.

Examples of the aromatic heterocyclic group include a 1-pyrrolyl group, a 2-pyrrolyl group, a 3-pyrrolyl group, a pyrazinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 2-dibenzofuranyl group, a 4-dibenzofuranyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-phenanthridinyl group, 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1,7-phenanthroline-2-yl group, a 1,7-phenanthroline-3-yl group, a 1,7-phenanthroline-4-yl group, a 1,7-phenanthroline-5-yl group, a 1,7-phenanthroline-6-yl group, a 1,7-phenanthroline-8-yl group, a 1,7-phenanthroline-9-yl group, a 1,7-phenanthroline-10-yl group, a 1,8-phenanthroline-2-yl group, a 1,8-phenanthroline-3-yl group, a 1,8-phenanthroline-4-yl group, a 1,8-phenanthroline-5-yl group, a 1,8-phenanthroline-6-yl group, a 1,8-phenanthroline-7-yl group, a 1,8-phenanthroline-9-yl group, a 1,8-phenanthroline-10-yl group, a 1,9-phenanthroline-2-yl group, a 1,9-phenanthroline-3-yl group, a 1,9-phenanthroline-4-yl group, a 1,9-phenanthroline-5-yl group, a 1,9-phenanthroline-6-yl group, a 1,9-phenanthroline-7-yl group, a 1,9-phenanthroline-8-yl group, a 1,9-phenanthroline-10-yl group, a 1,10-phenanthroline-2-yl group, a 1,10-phenanthroline-3-yl group, a 1,10-phenanthroline-4-yl group, a 1,10-phenanthroline-5-yl group, a 2,9-phenanthroline-1-yl group, a 2,9-phenanthroline-3-yl group, a 2,9-phenanthroline-4-yl group, a 2,9-phenanthroline-5-yl group, a 2,9-phenanthroline-6-yl group, a 2,9-phenanthroline-7-yl group, a 2,9-phenanthroline-8-yl group, a 2,9-phenanthroline-10-yl group, a 2,8-phenanthroline-1-yl group, a 2,8-phenanthroline-3-yl group, a 2,8-phenanthroline-4-yl group, a 2,8-phenanthroline-5-yl group, a 2,8-phenanthroline-6-yl group, a 2,8-phenanthroline-7-yl group, a 2,8-phenanthroline-9-yl group, a 2,8-phenanthroline-10-yl group, a 2,7-phenanthroline-1-yl group, a 2,7-phenanthroline-3-yl group, a 2,7-phenanthroline-4-yl group, a 2,7-phenanthroline-5-yl group, a 2,7-phenanthroline-6-yl group, a 2,7-phenanthroline-8-yl group, a 2,7-phenanthroline-9-yl group, a 2,7-phenanthroline-10-yl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrole-1-yl group, a 2-methylpyrrole-3-yl group, a 2-methylpyrrole-4-yl group, a 2-methylpyrrole-5-yl group, a 3-methylpyrrole-1-yl group, a 3-methylpyrrole-2-yl group, a 3-methylpyrrole-4-yl group, a 3-methylpyrrole-5-yl group, a 2-t-butylpyrrole-4-yl group, a 3-(2-phenylpropyl)pyrrole-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a 2-thienyl group, a 3-thienyl group, a 2-benzothiophenyl group, a 3-thiophenyl group, a 4-thiophenyl group, a 5-thiophenyl group, a 6-thiophenyl group, a 7-thiophenyl group, a 1-isothiophenyl group, a 3-isothiophenyl group, a 4-isothiophenyl group, a 5-isothiophenyl group, a 6-isothiophenyl group, a 7-thiophenyl group, a 2-dibenzothiophenyl group, and a 4-dibenzothiophenyl group.

Examples of the substituent include an aryl group, such as a phenyl group, a 9,9-dimethylfluorenyl group, and a 9,9-dioctylfluorenyl group, a heteroaryl group, such as a pyridyl group, a pyrimidyl group, and a dibenzofuranyl group, an alkyl group having 1 to 10 carbon atoms, a halogen atom, a cyano group, and a combination thereof.

Of the above, preferred are a 1-pyrrolyl group, a 2-pyrrolyl group, a 3-pyrrolyl group, a 2-dibenzofuranyl group, a 4-dibenzofuranyl group, a 2-dibenzothiophenyl group, a 4-dibenzothiophenyl group, a 2,2-bipyridin-4-yl group, a 2, 2-bipyridin-6-yl group, a 2,2′:2′,2″-terpyridin-4′-yl group, a 2,2′:6′,2″-terpyridin-4′-phenyl-4-yl group, a benzothiazol-4-yl group, a benzothiazol-5-yl group, a trans-stilben-4-yl group, a 2,2′-bithiophen-3-yl group, a 2,2′-bithiophen-5-yl group, a 3,4-ethylenedioxythiophen-2-yl group, a 7-(2-thienyl)-2,1,3-benzothiadiazol-4-(2-thienyl-5-yl) group, and divalent residues thereof.

The substituted or unsubstituted arylamino group for Ar₁ is bonded to the main chain and the side chain via the aryl group bonded to the nitrogen atom, bonded thereto via the nitrogen atom and the aryl group bonded to the nitrogen atom, or the diarylamino group is bonded to the nitrogen atom of the side chain. In any of these cases, the aryl group is preferably selected from the aromatic hydrocarbon ring group having 6 to 60 ring carbon atoms mentioned above.

The aromatic hydrocarbon ring group for Ar₂′ has preferably 6 to 60, more preferably 6 to 30, and further more preferably 6 to 15 ring carbon atoms. Examples of the substituted or unsubstituted aromatic hydrocarbon group having 6 to 60 ring carbon atoms for Ar₂′ are the same as those mentioned above with respect to the aromatic hydrocarbon ring group for Ar₁. Ar₂′ is preferably a phenyl group.

Examples of the substituted or unsubstituted aromatic hydrocarbon group having 6 to 60 ring carbon atoms and the substituted or unsubstituted aromatic heterocyclic group having 5 to 60 ring atoms for A are the same as those mentioned above with respect to Ar₁.

The subscript x in formula (HTG2e) is preferably an integer of 1 to 3 and more preferably 1 or 2.

Examples of the substituent X are shown below, although not limited thereto. In the following examples, any of hydrogen atoms may be replaced by the bonding site.

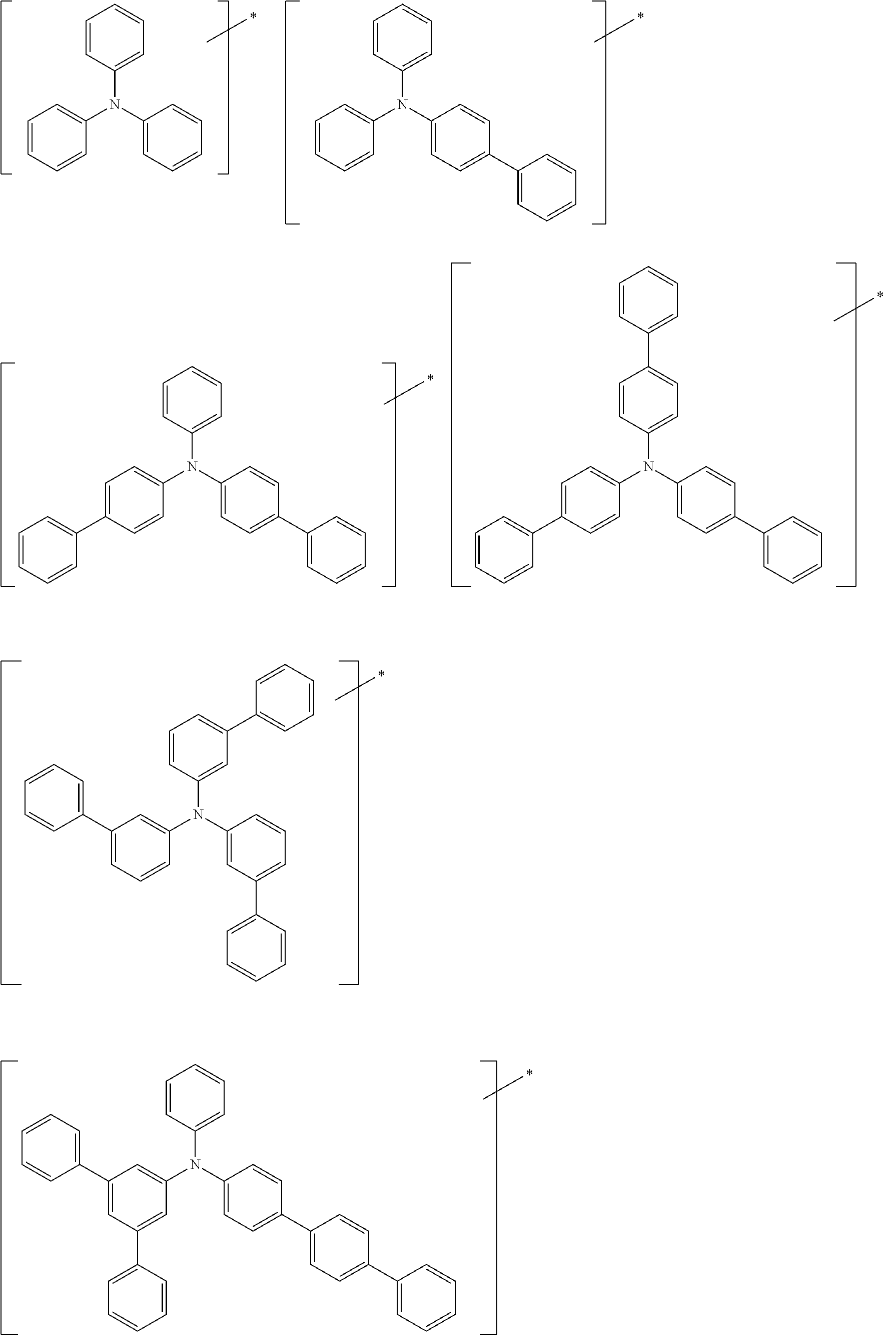

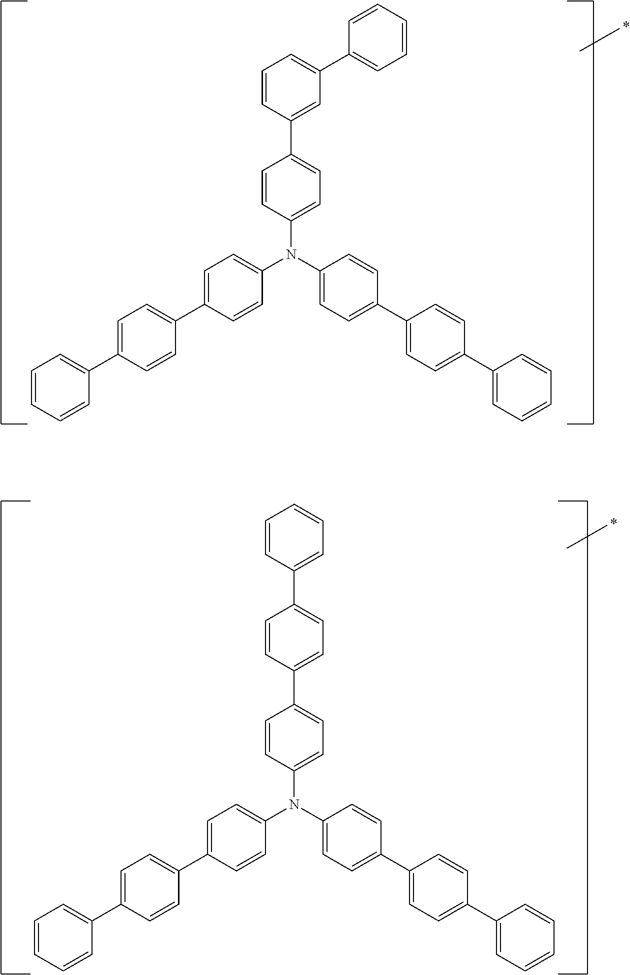

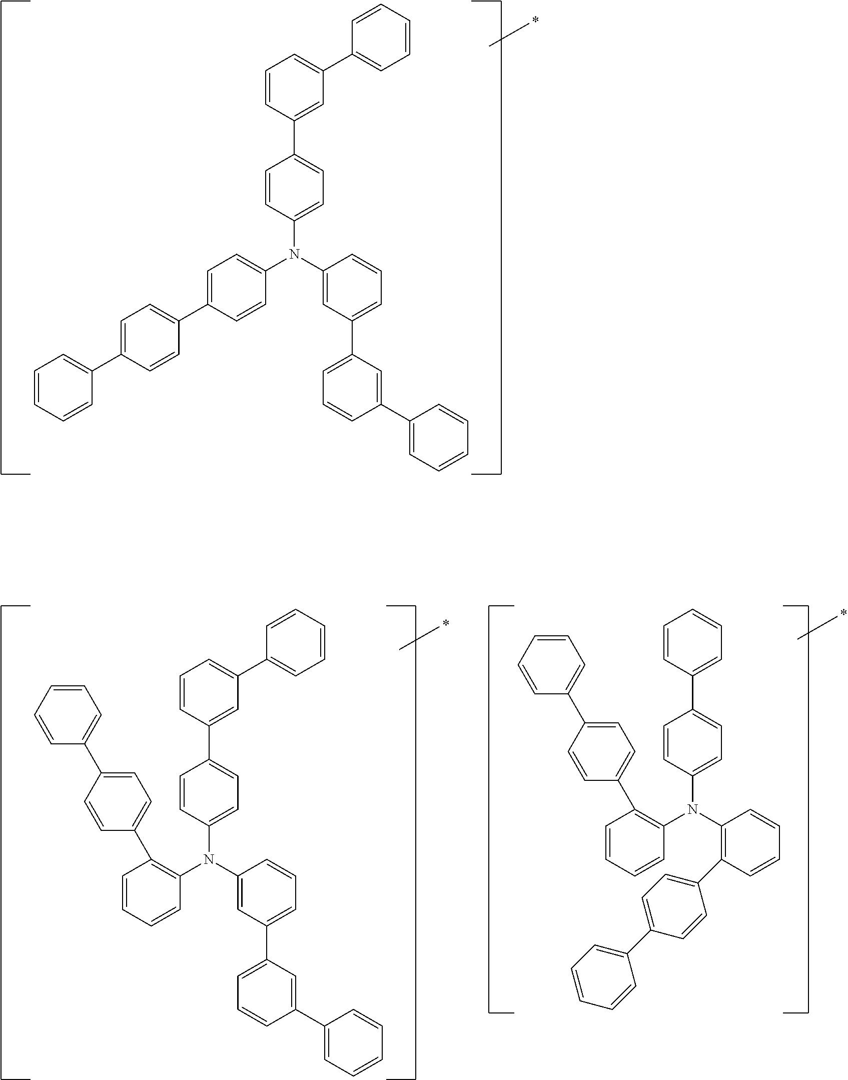

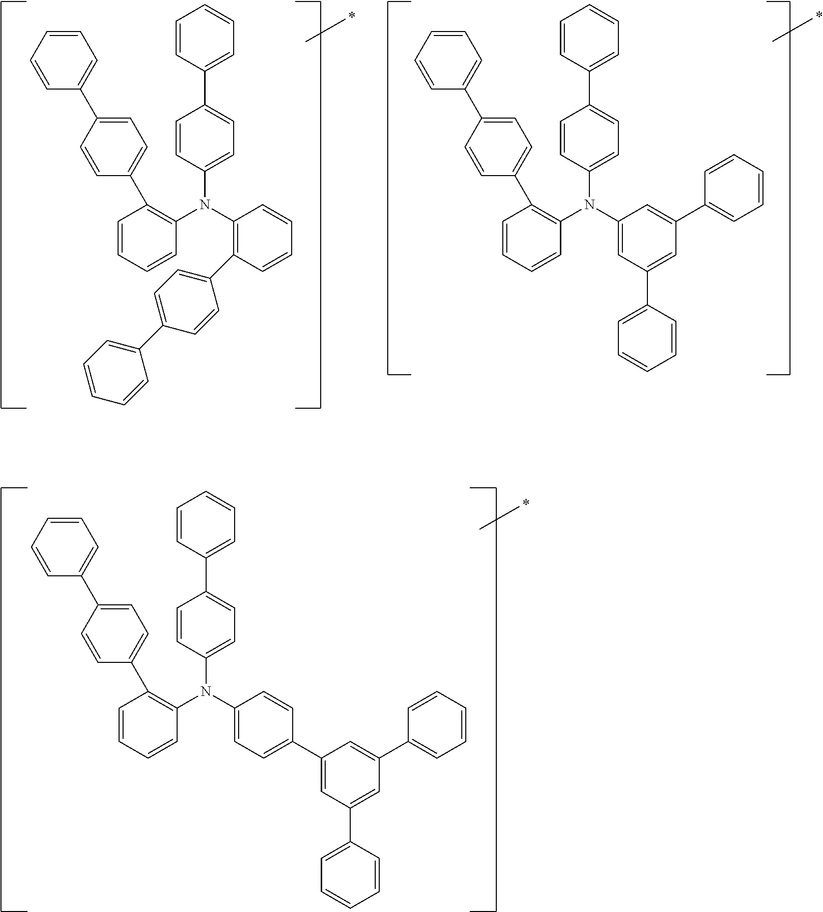

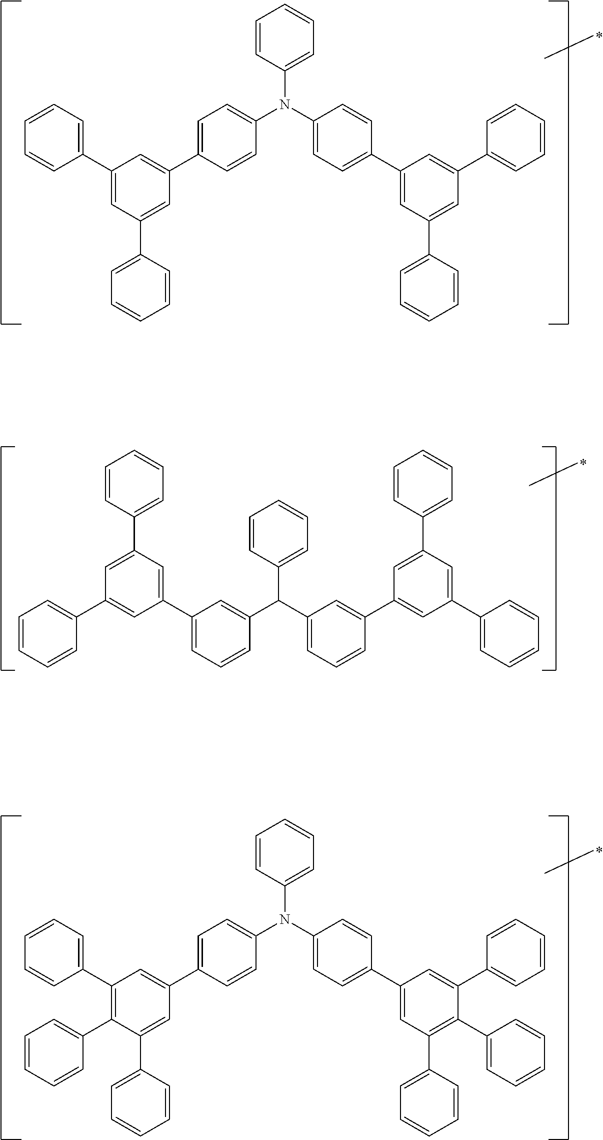

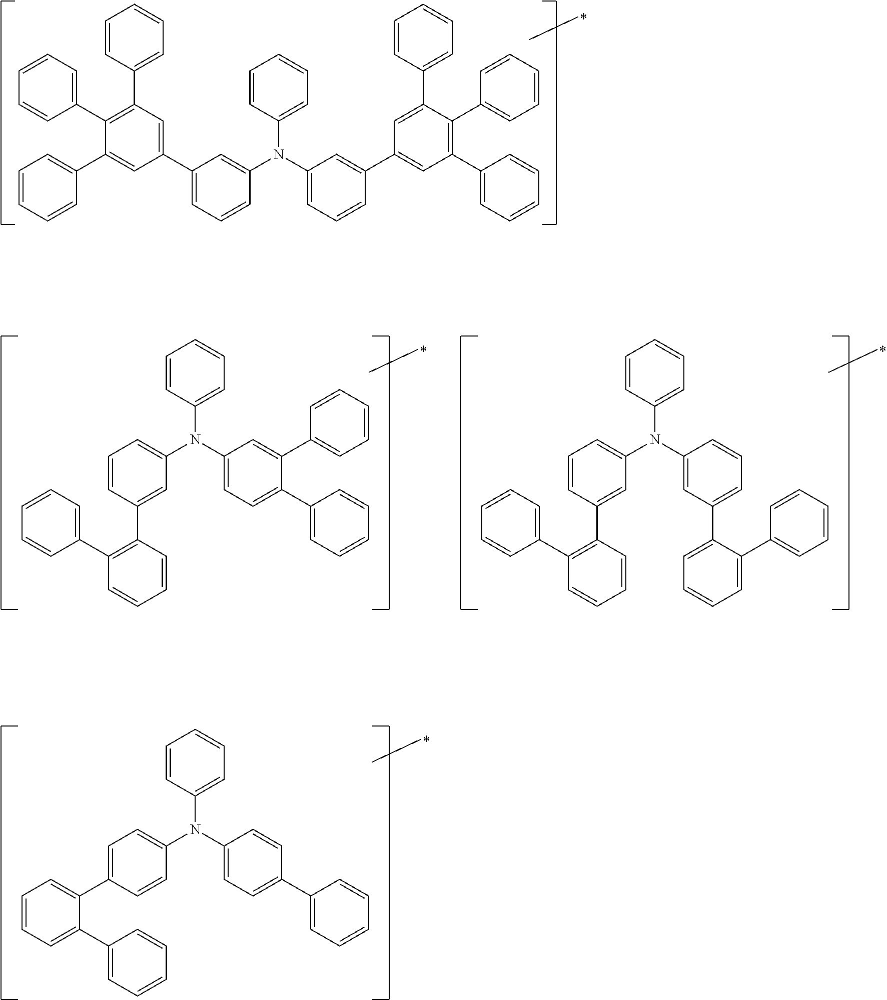

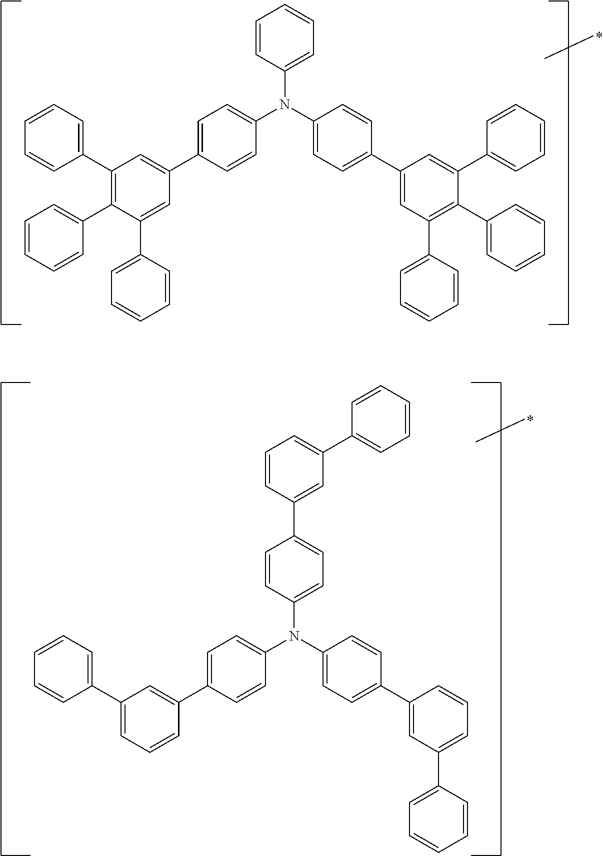

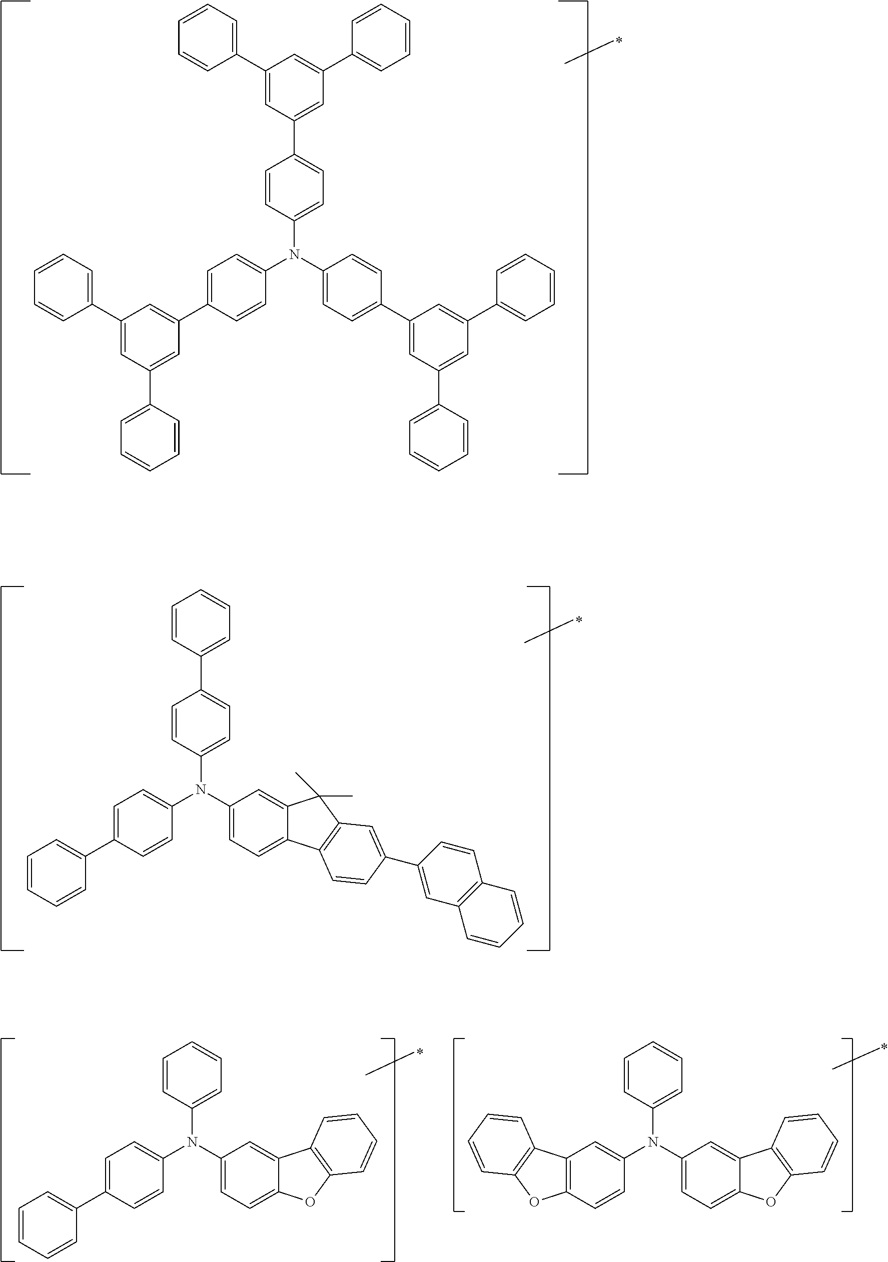

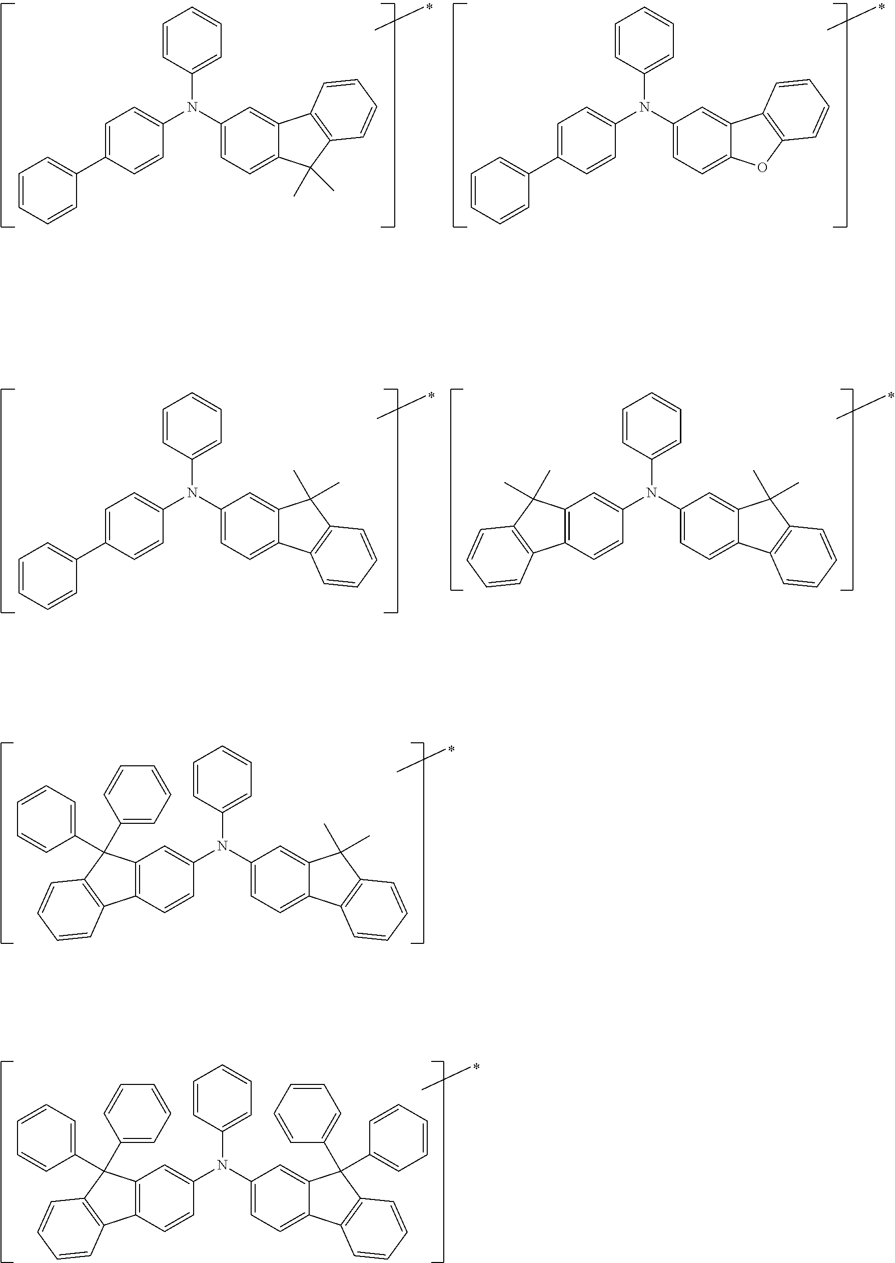

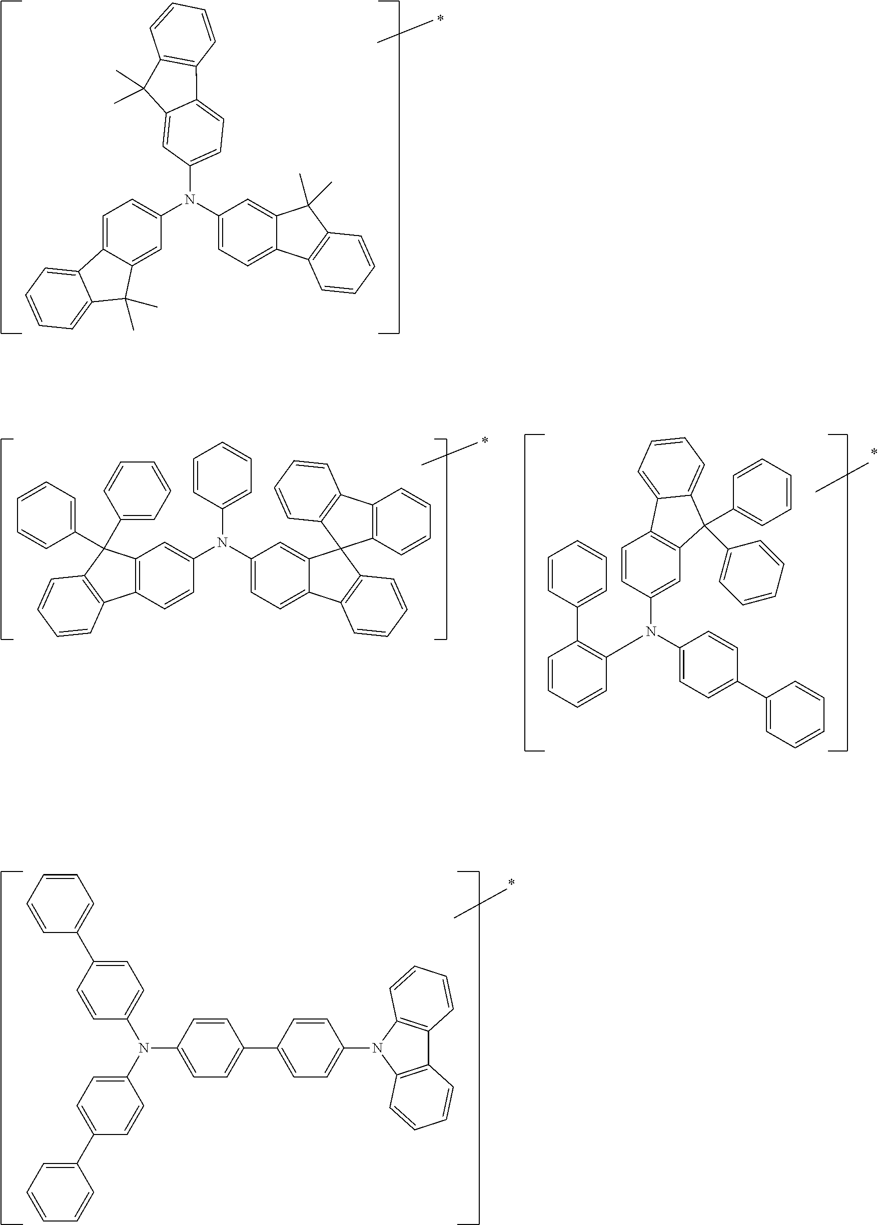

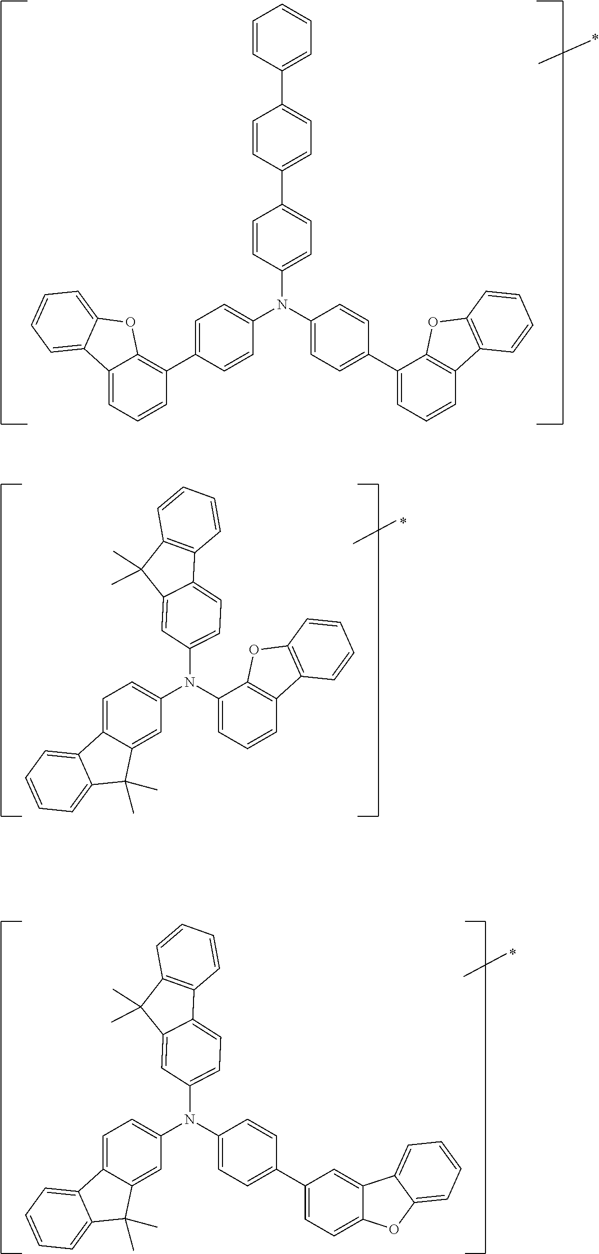

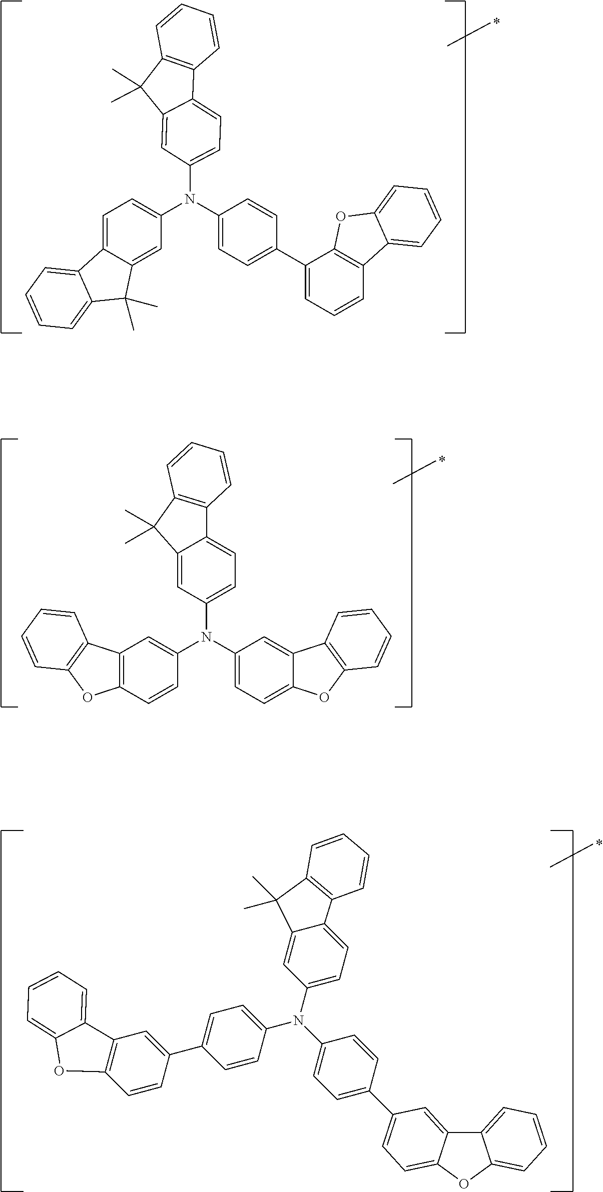

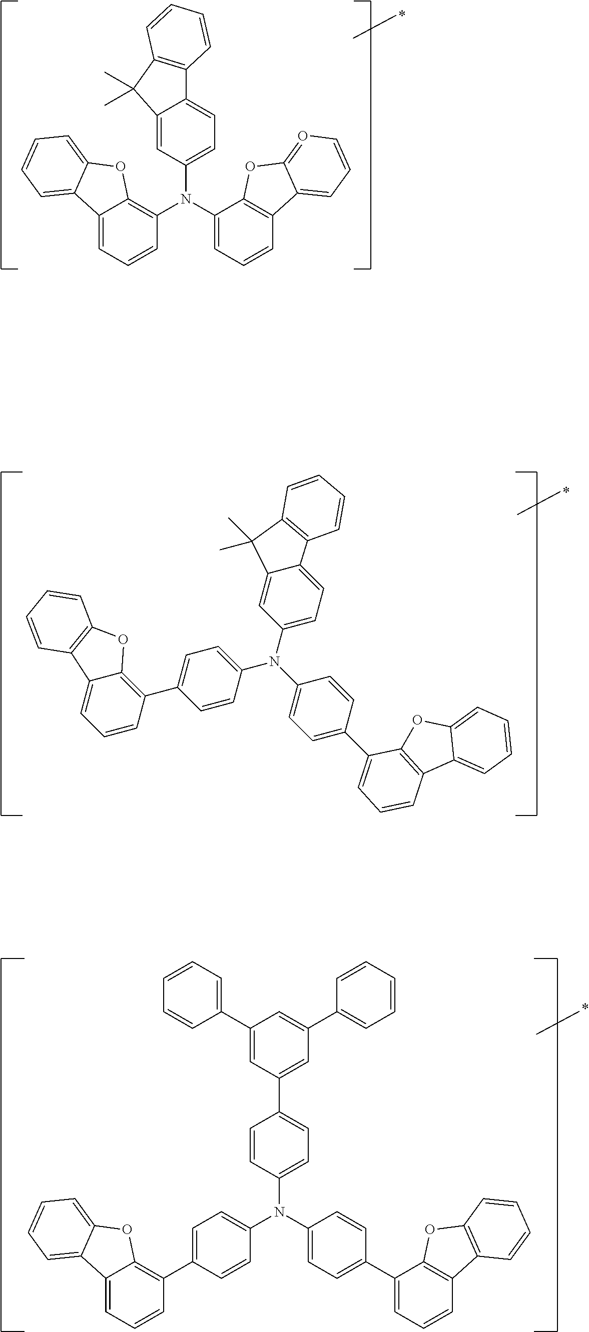

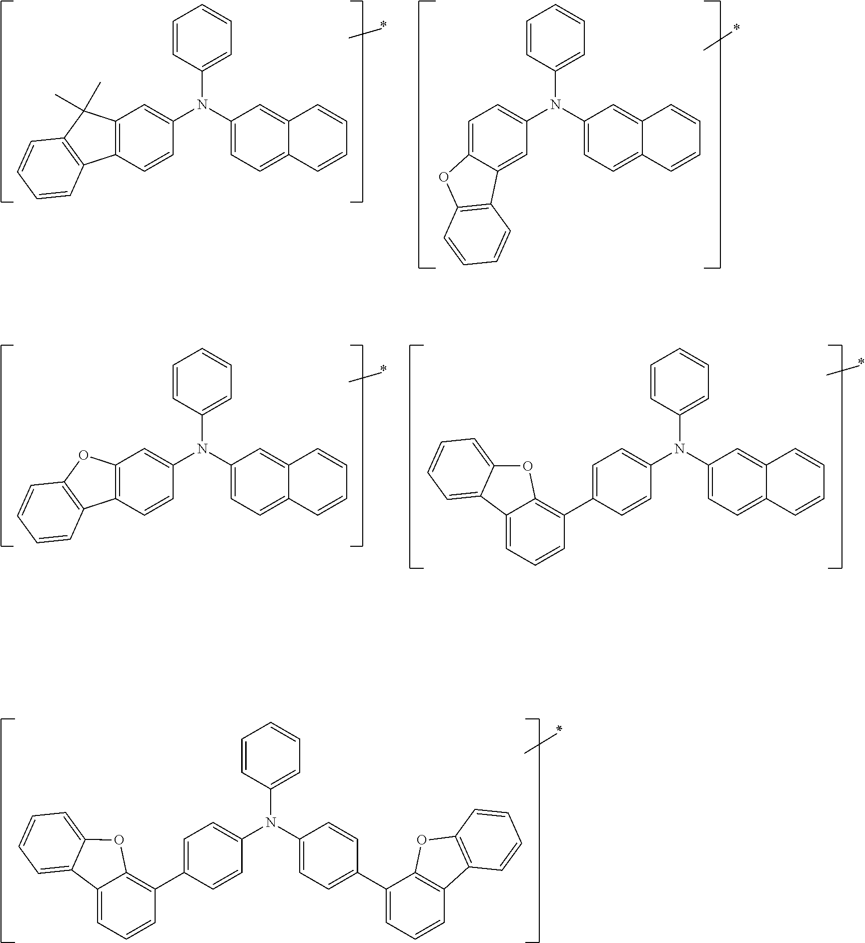

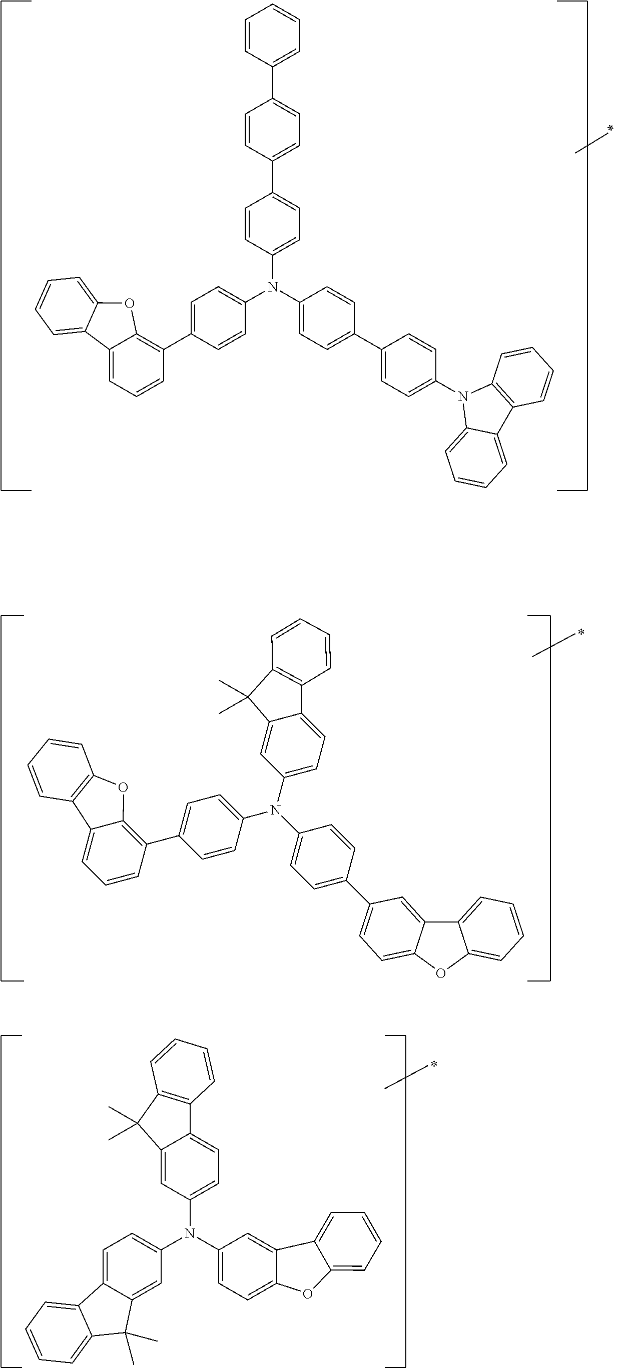

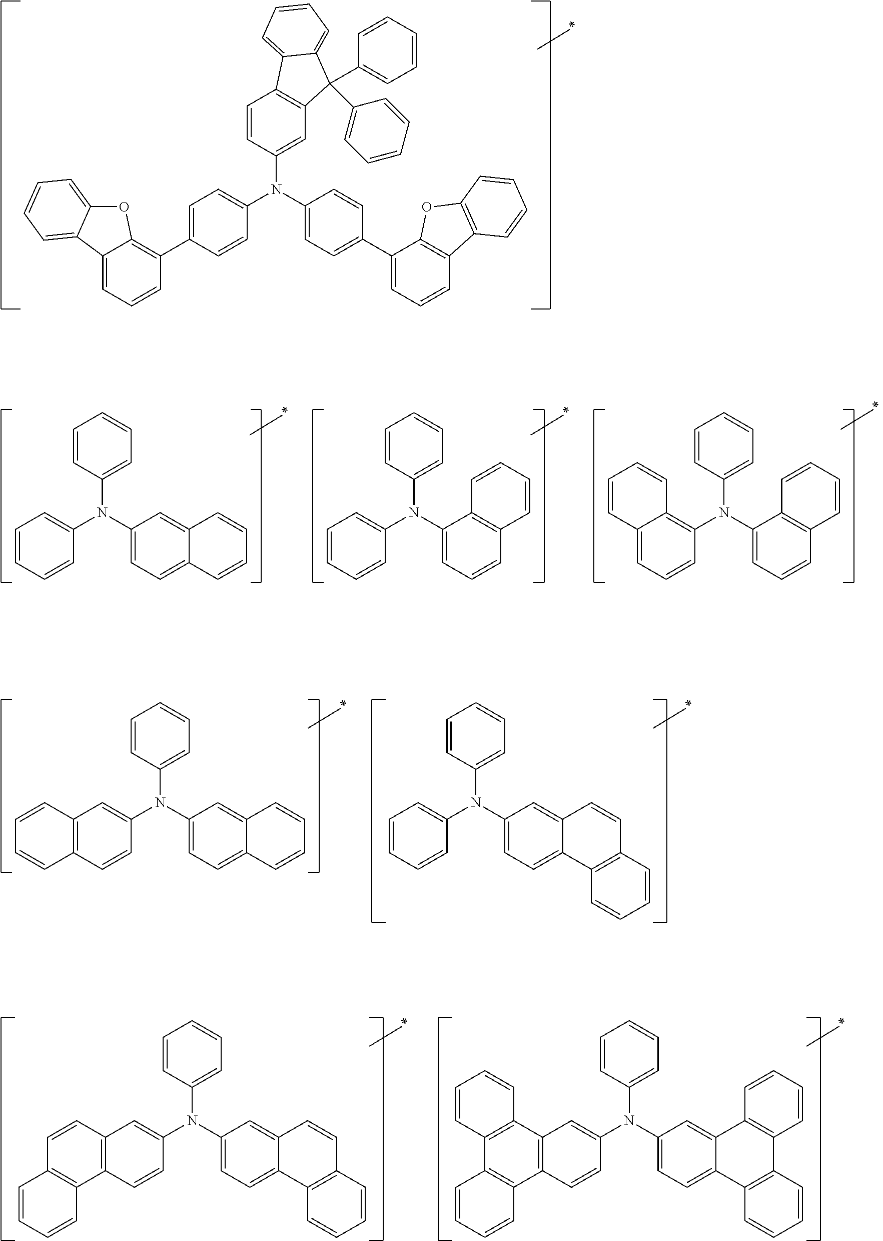

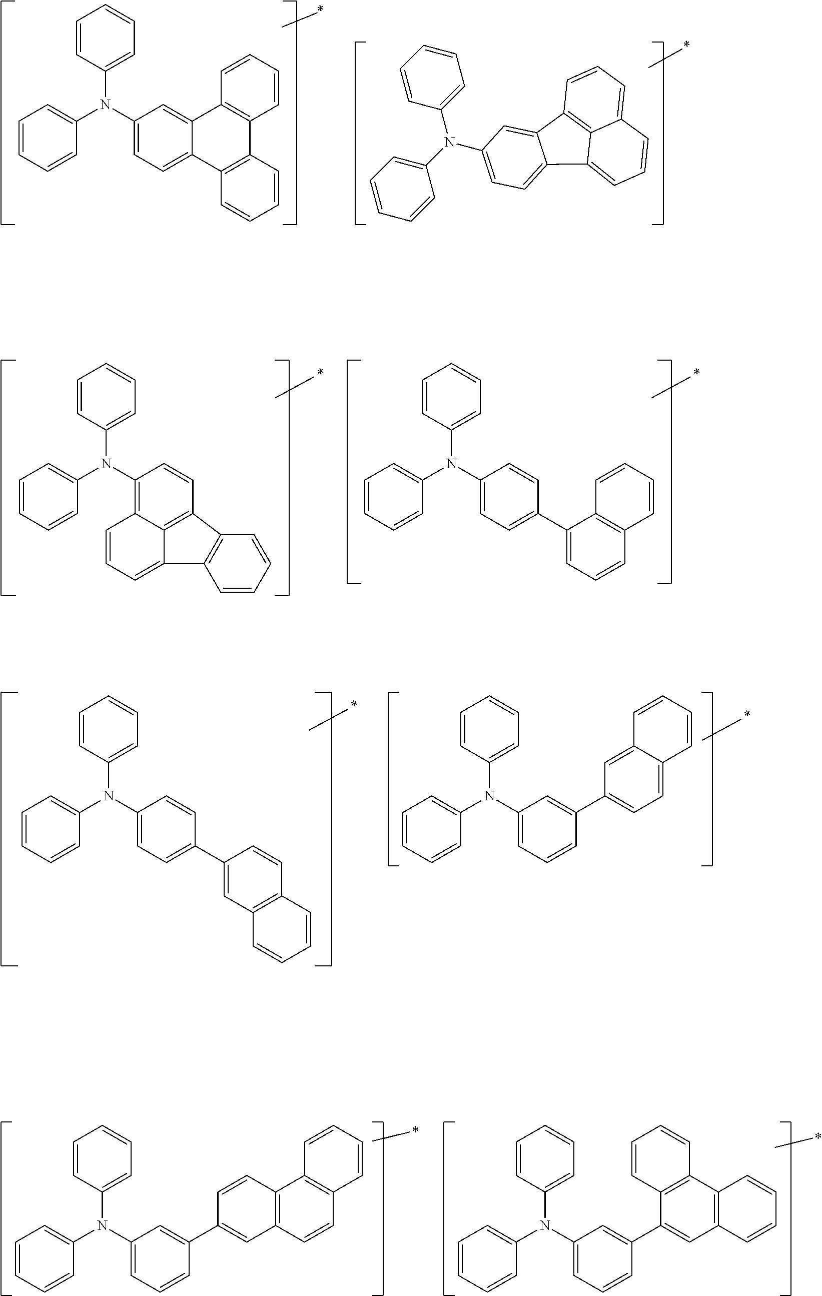

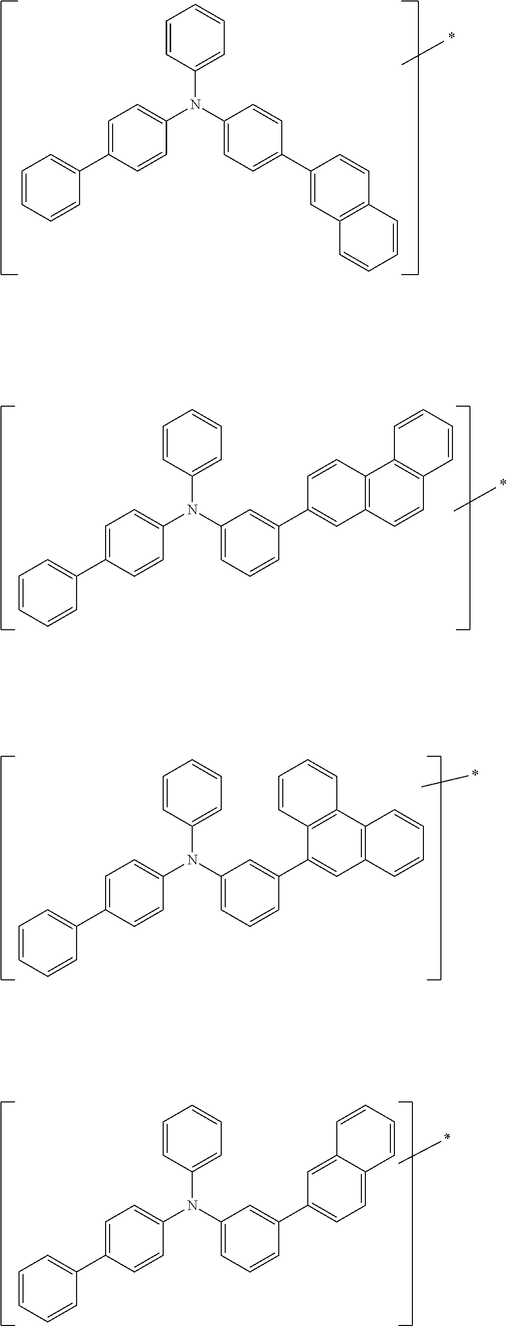

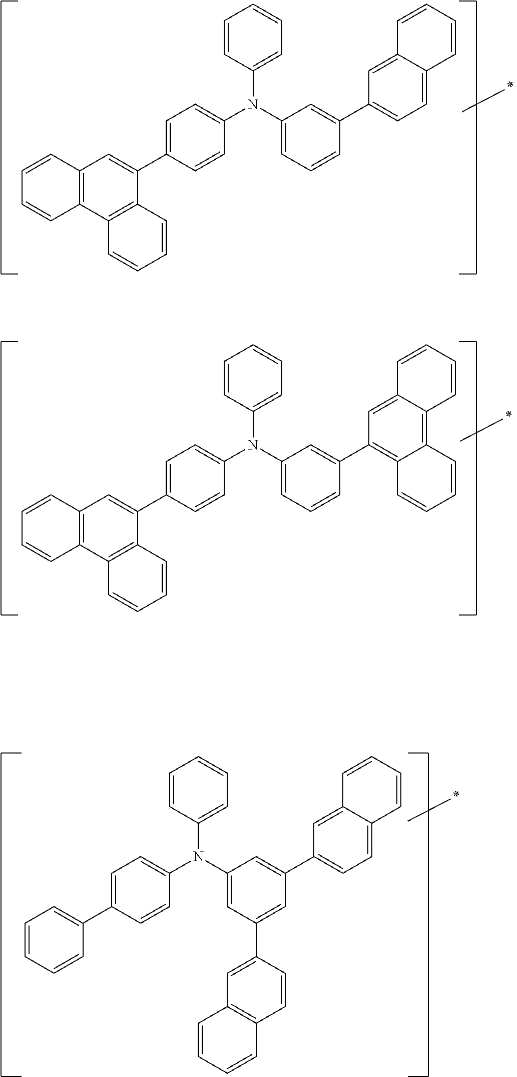

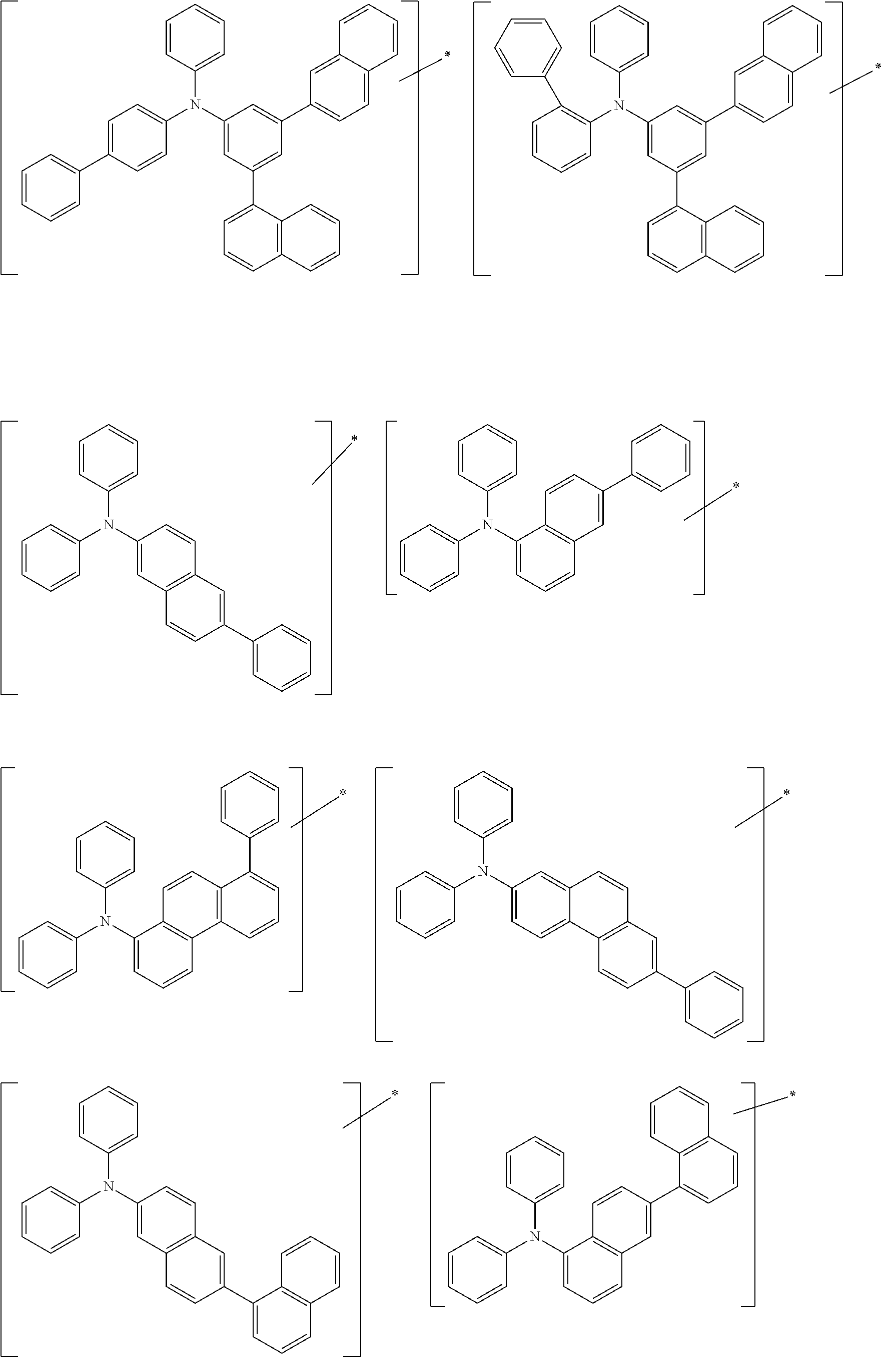

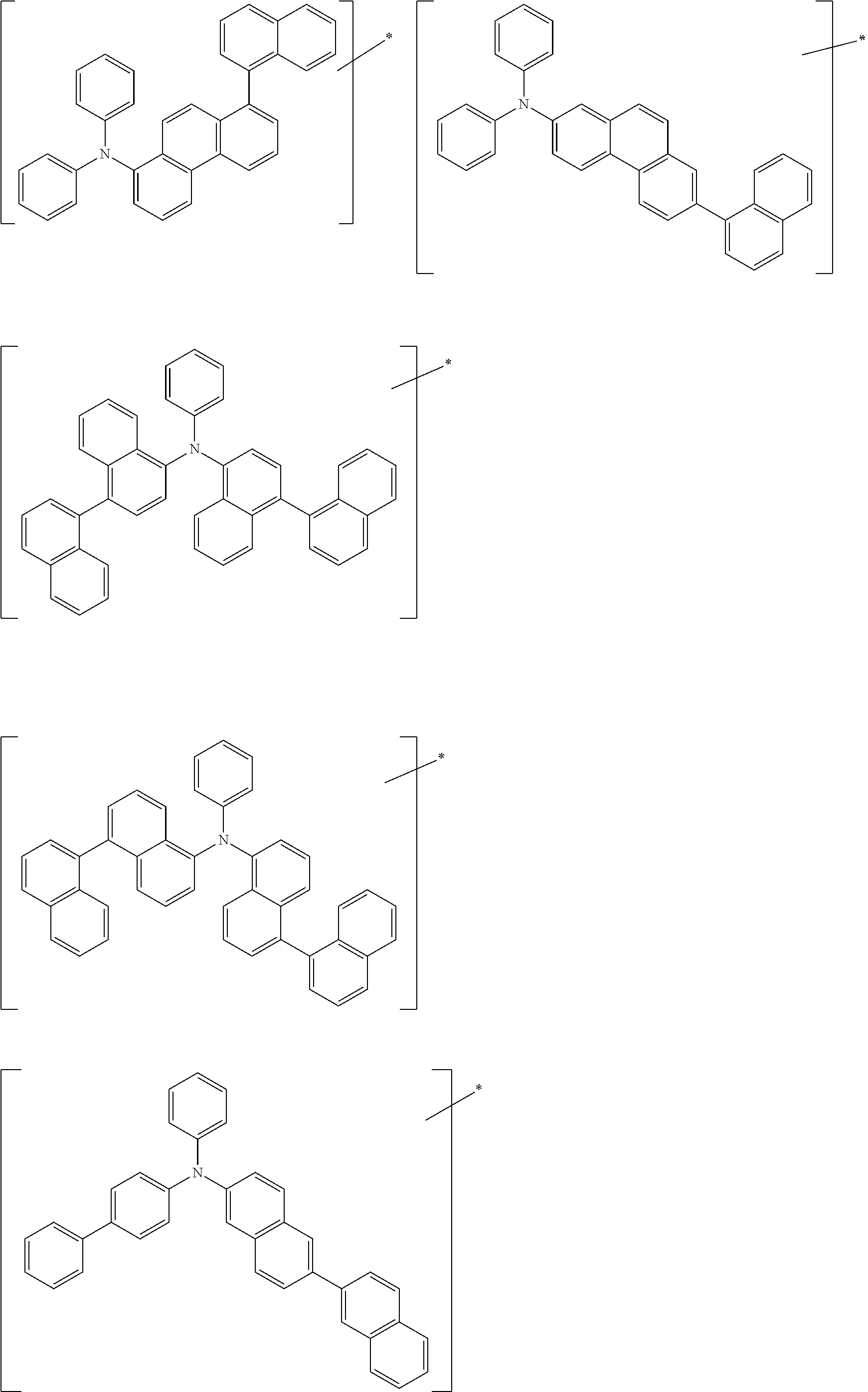

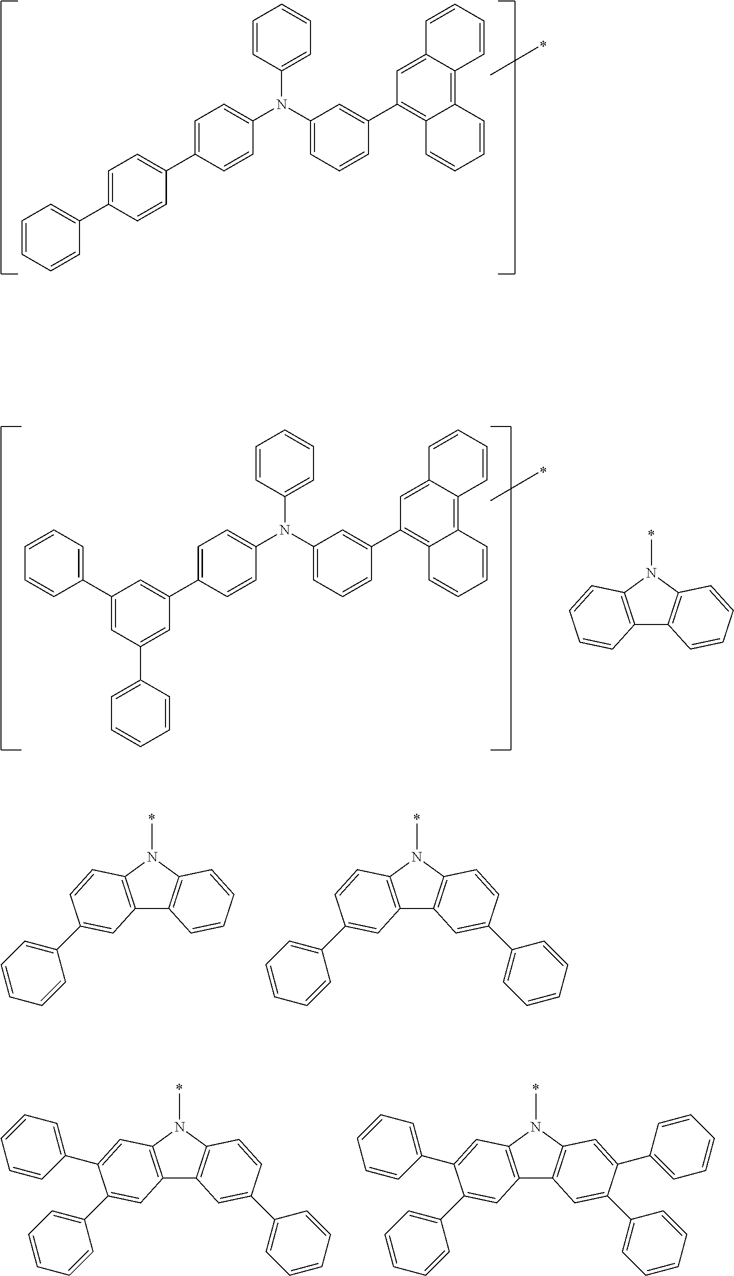

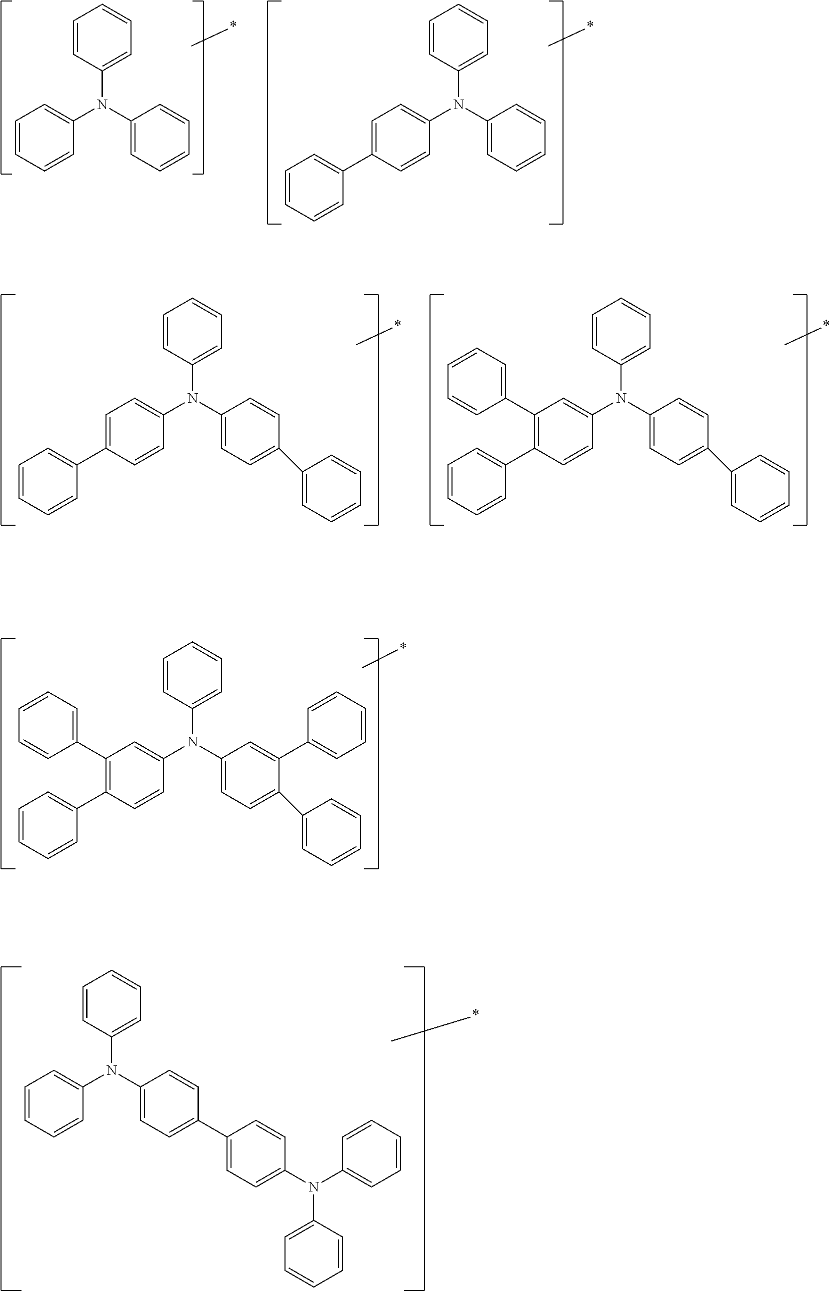

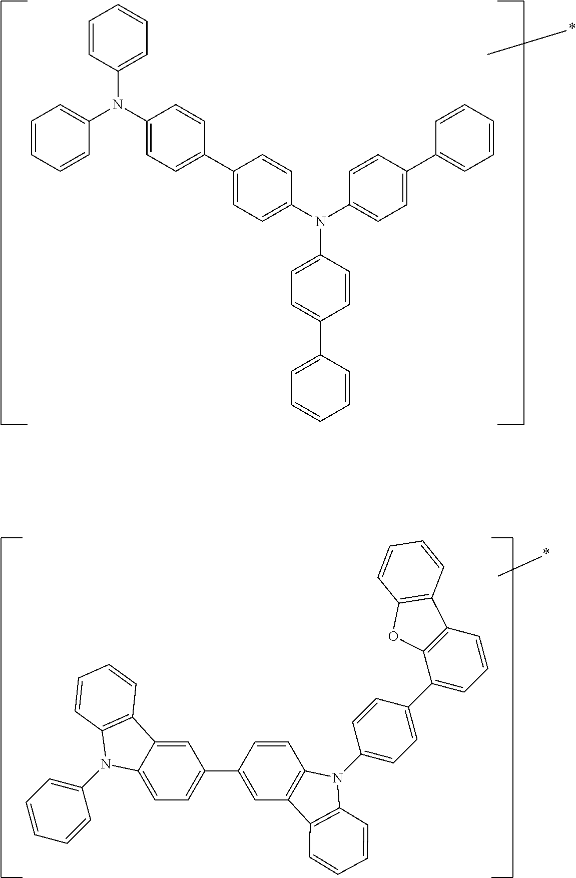

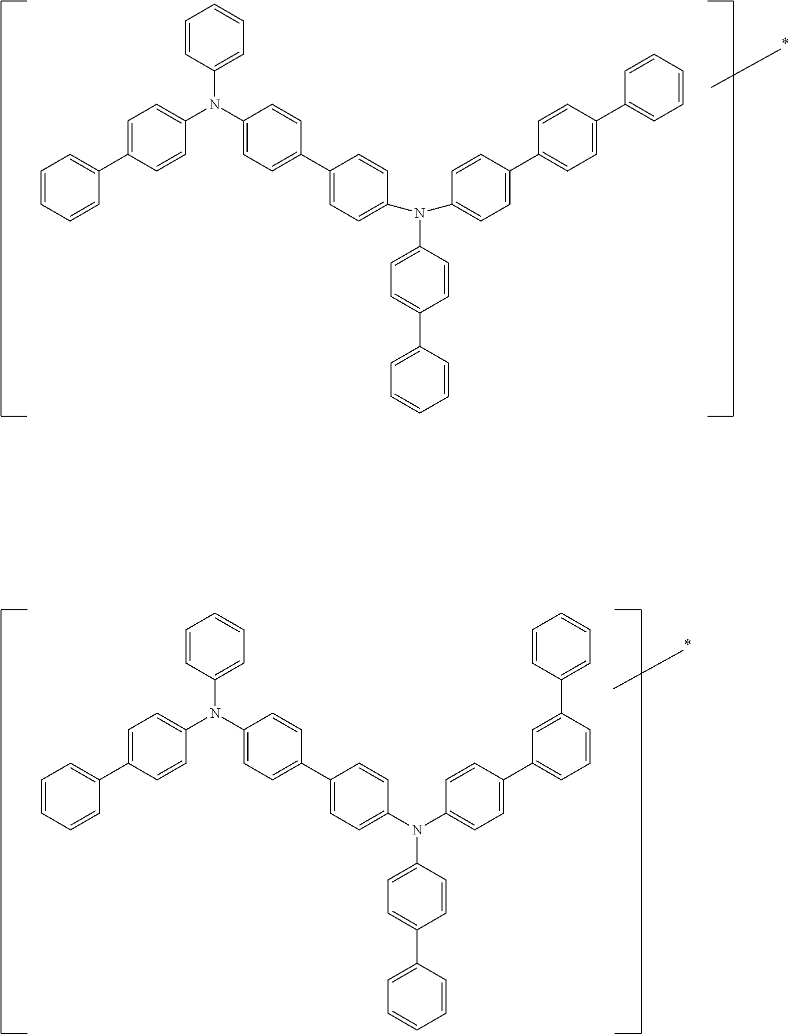

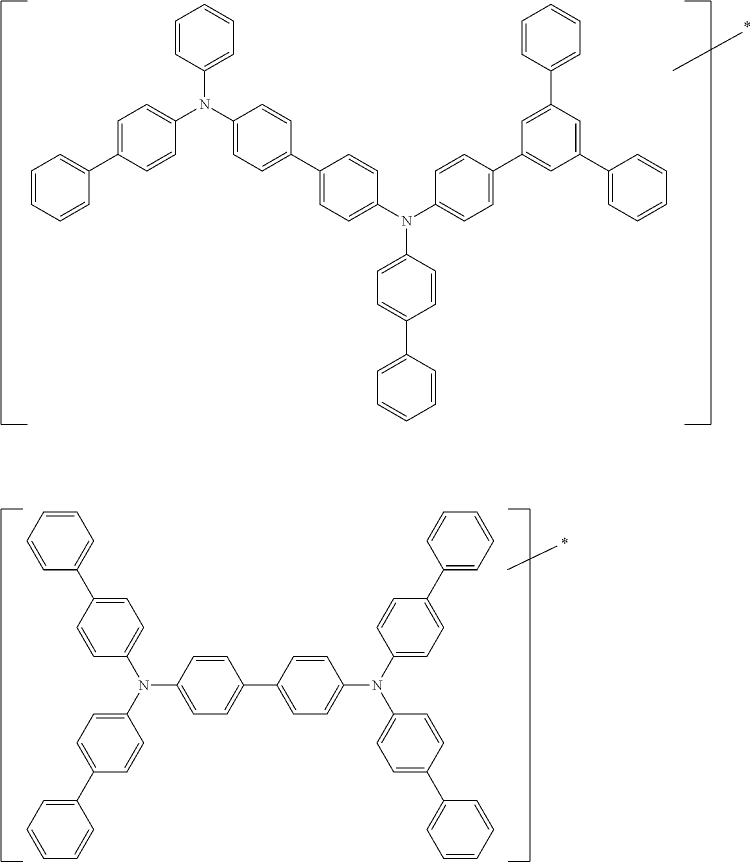

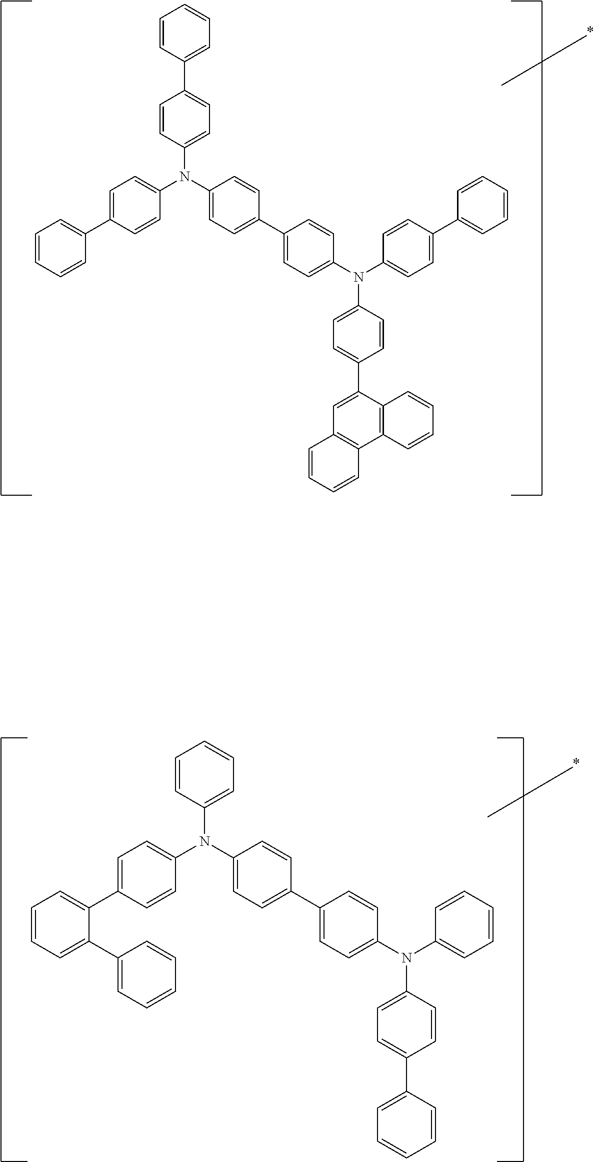

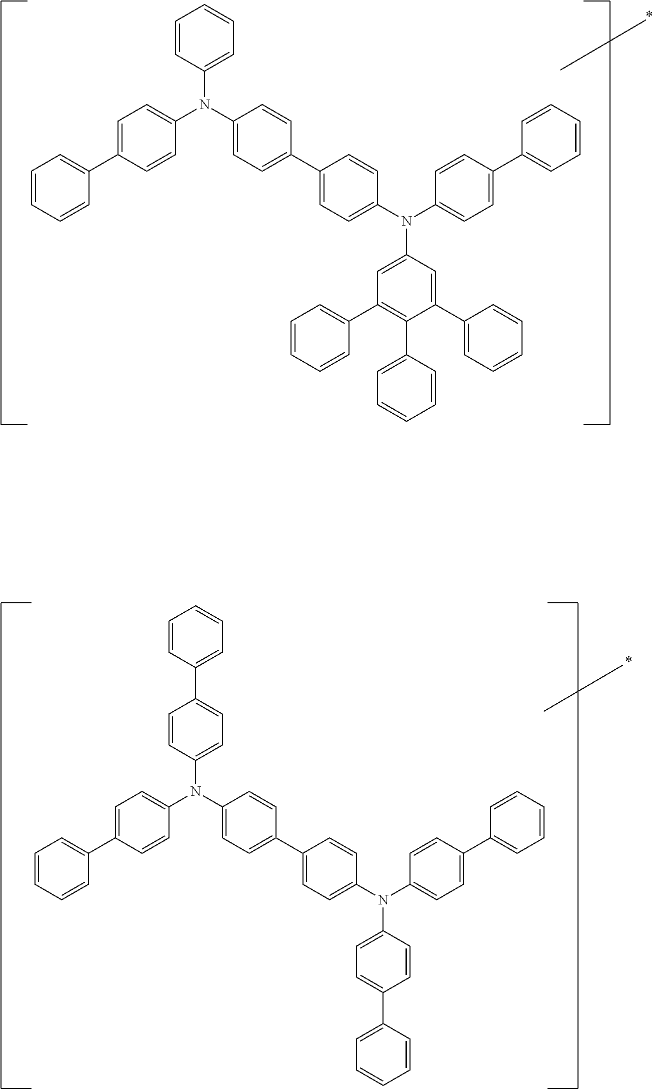

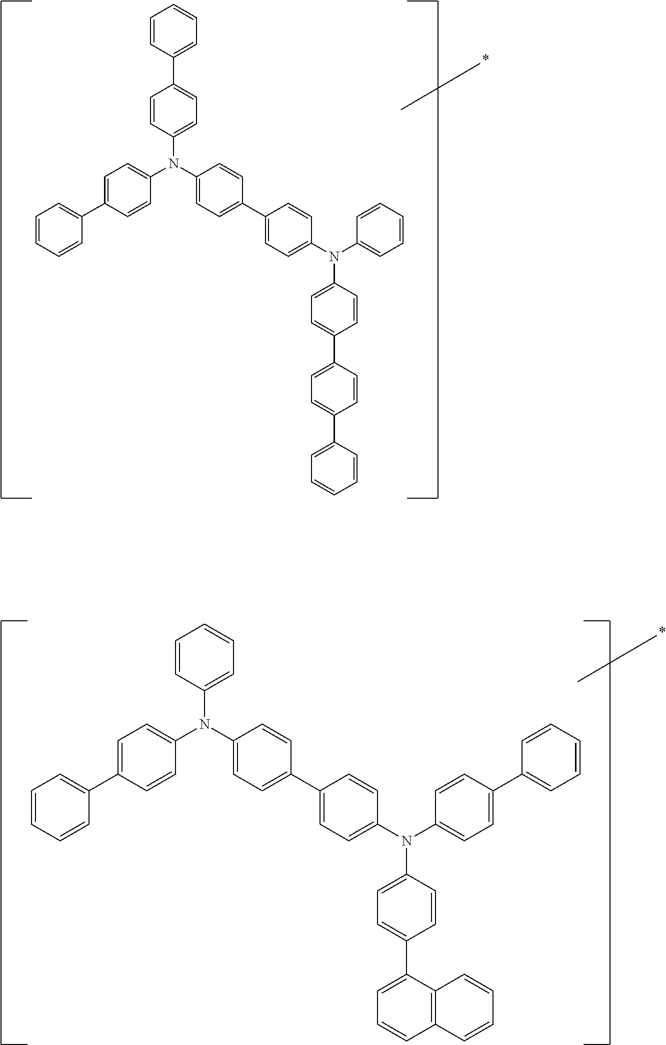

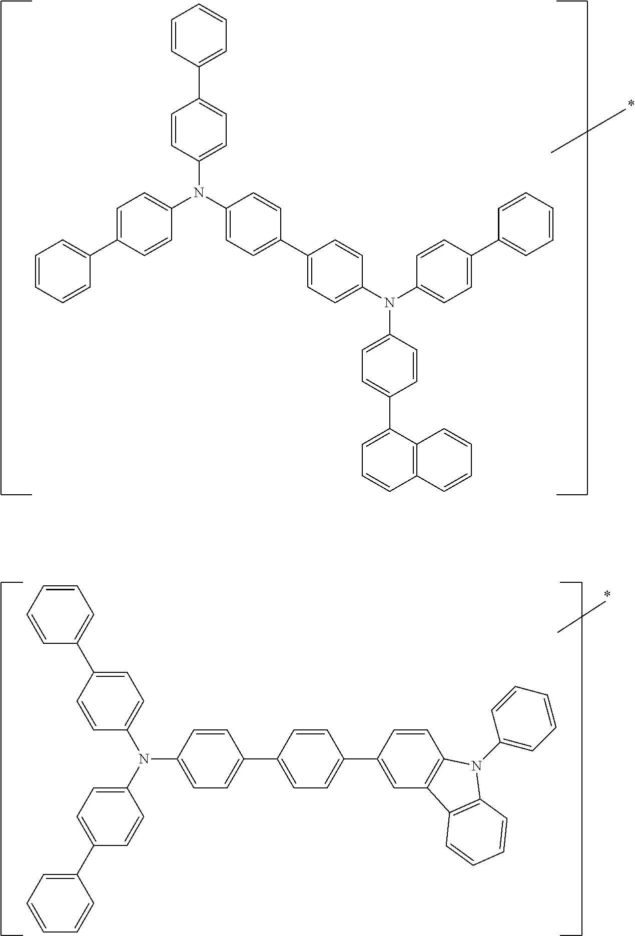

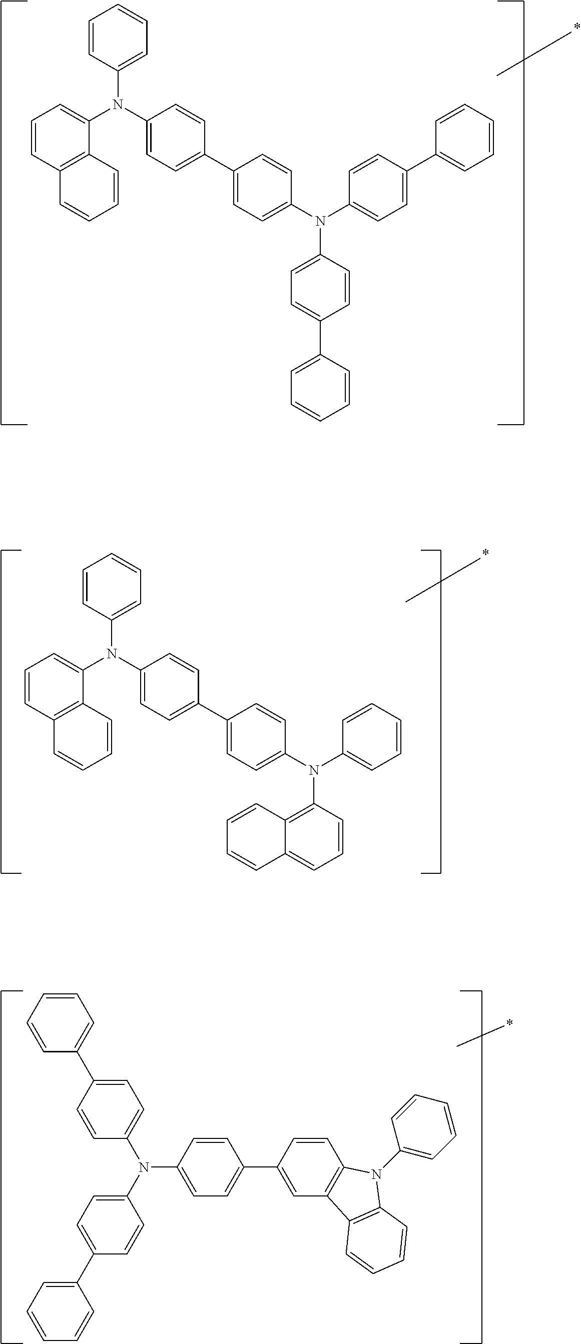

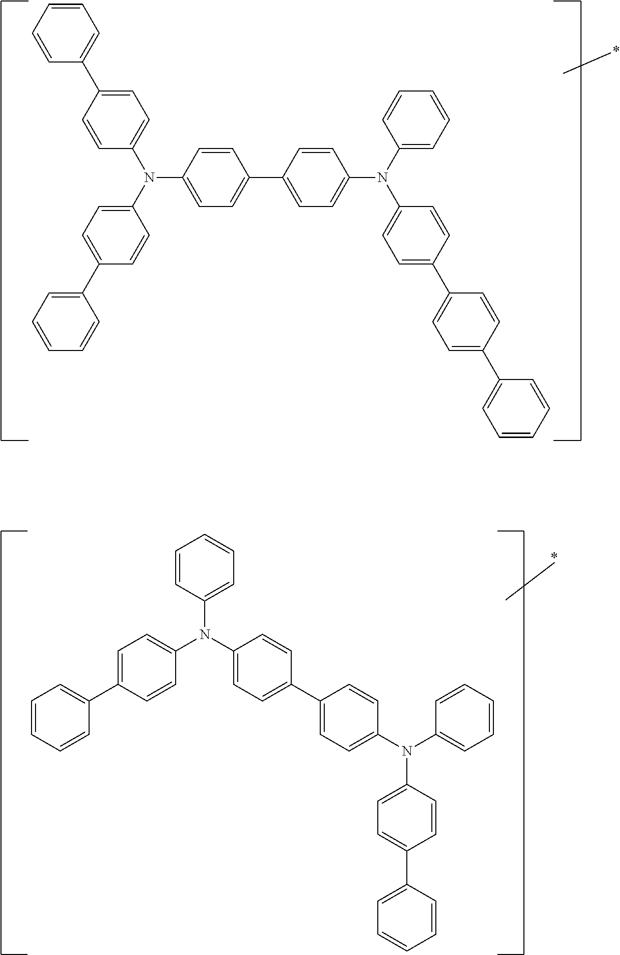

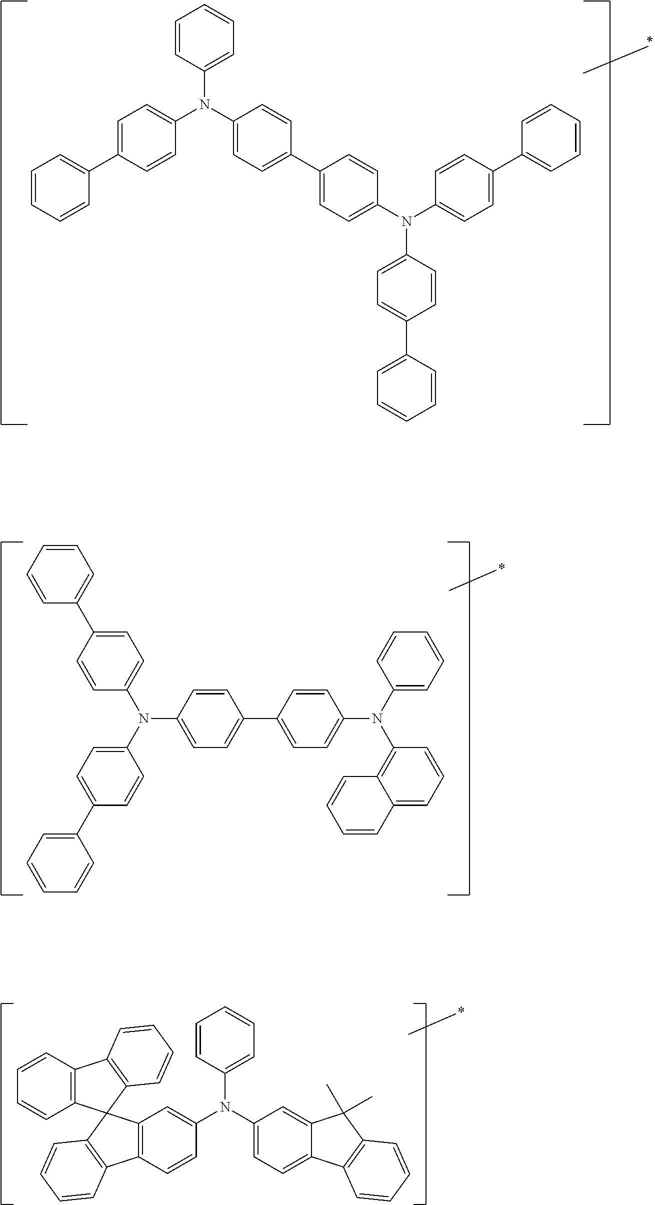

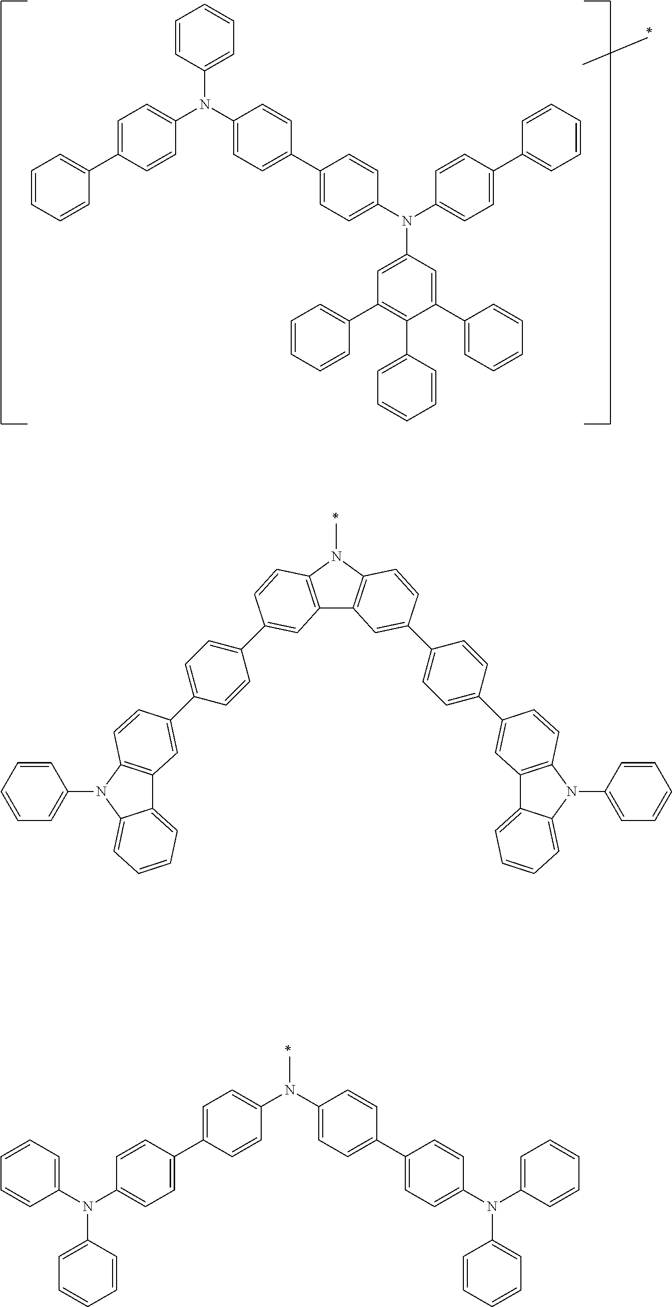

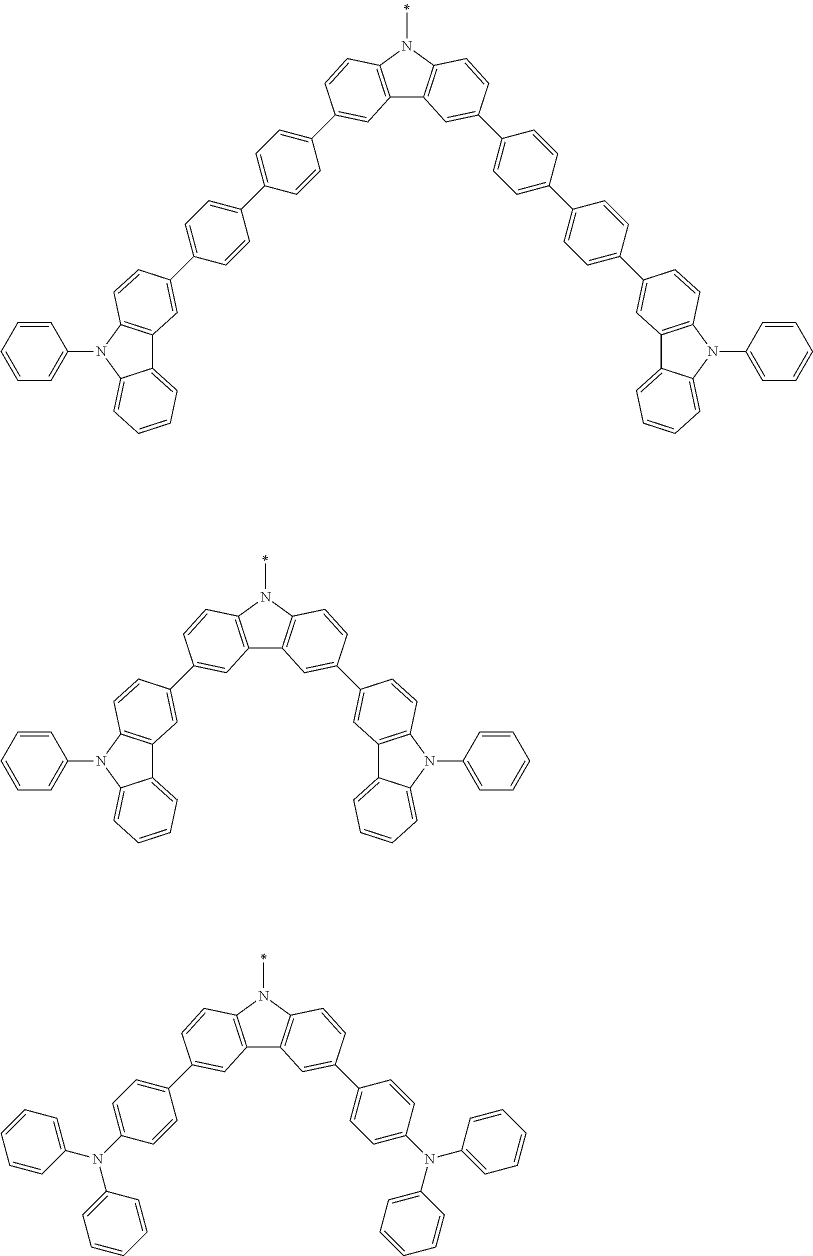 [SMILES: Cn1c2ccc(-c3ccc(-c4ccc(-c5ccc6c(c5)c5ccccc5n6-c5ccccc5)cc4)cc3)cc2c2cc(-c3ccc(-c4ccc(-c5ccc6c(c5)c5ccccc5n6-c5ccccc5)cc4)cc3)ccc21 ; Cn1c2ccc(-c3ccc4c(c3)c3ccccc3n4-c3ccccc3)cc2c2cc(-c3ccc4c(c3)c3ccccc3n4-c3ccccc3)ccc21 ; Cn1c2ccc(-c3ccc(N(c4ccccc4)c4ccccc4)cc3)cc2c2cc(-c3ccc(N(c4ccccc4)c4ccccc4)cc3)ccc21]

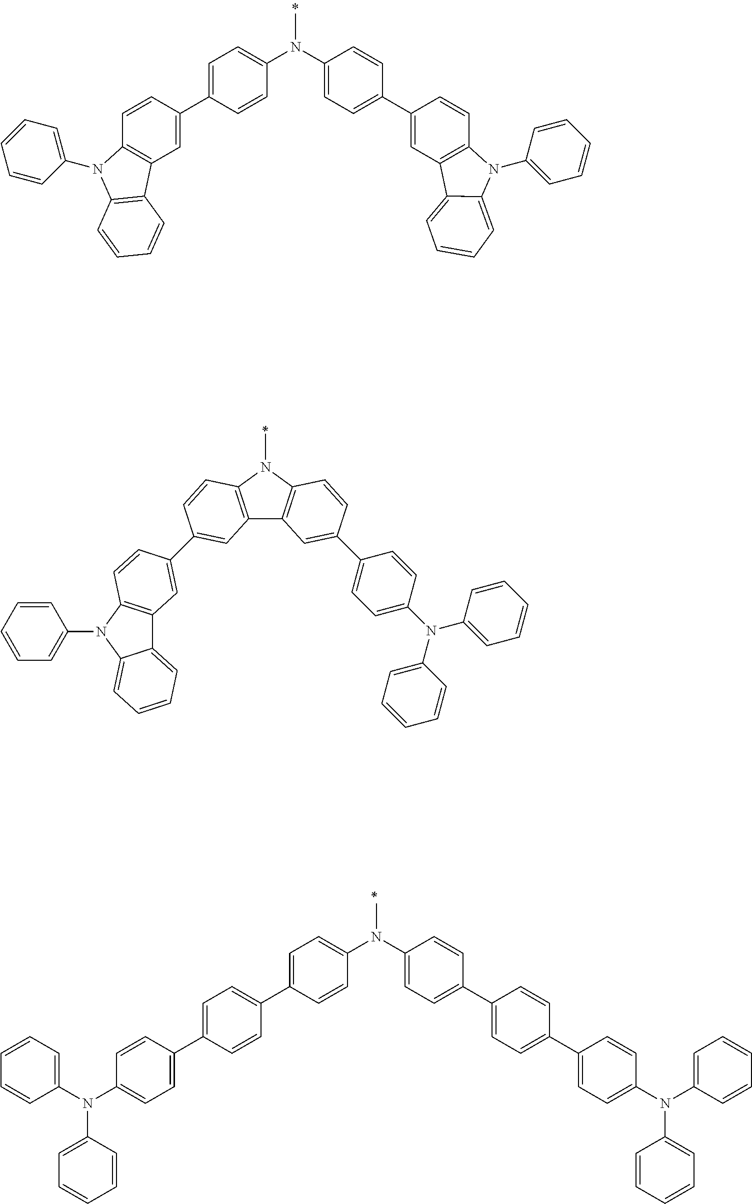 [SMILES: CN(c1ccc(-c2ccc3c(c2)c2ccccc2n3-c2ccccc2)cc1)c1ccc(-c2ccc3c(c2)c2ccccc2n3-c2ccccc2)cc1 ; Cn1c2ccc(-c3ccc(N(c4ccccc4)c4ccccc4)cc3)cc2c2cc(-c3ccc4c(c3)c3ccccc3n4-c3ccccc3)ccc21 ; CN(c1ccc(-c2ccc(-c3ccc(N(c4ccccc4)c4ccccc4)cc3)cc2)cc1)c1ccc(-c2ccc(-c3ccc(N(c4ccccc4)c4ccccc4)cc3)cc2)cc1]

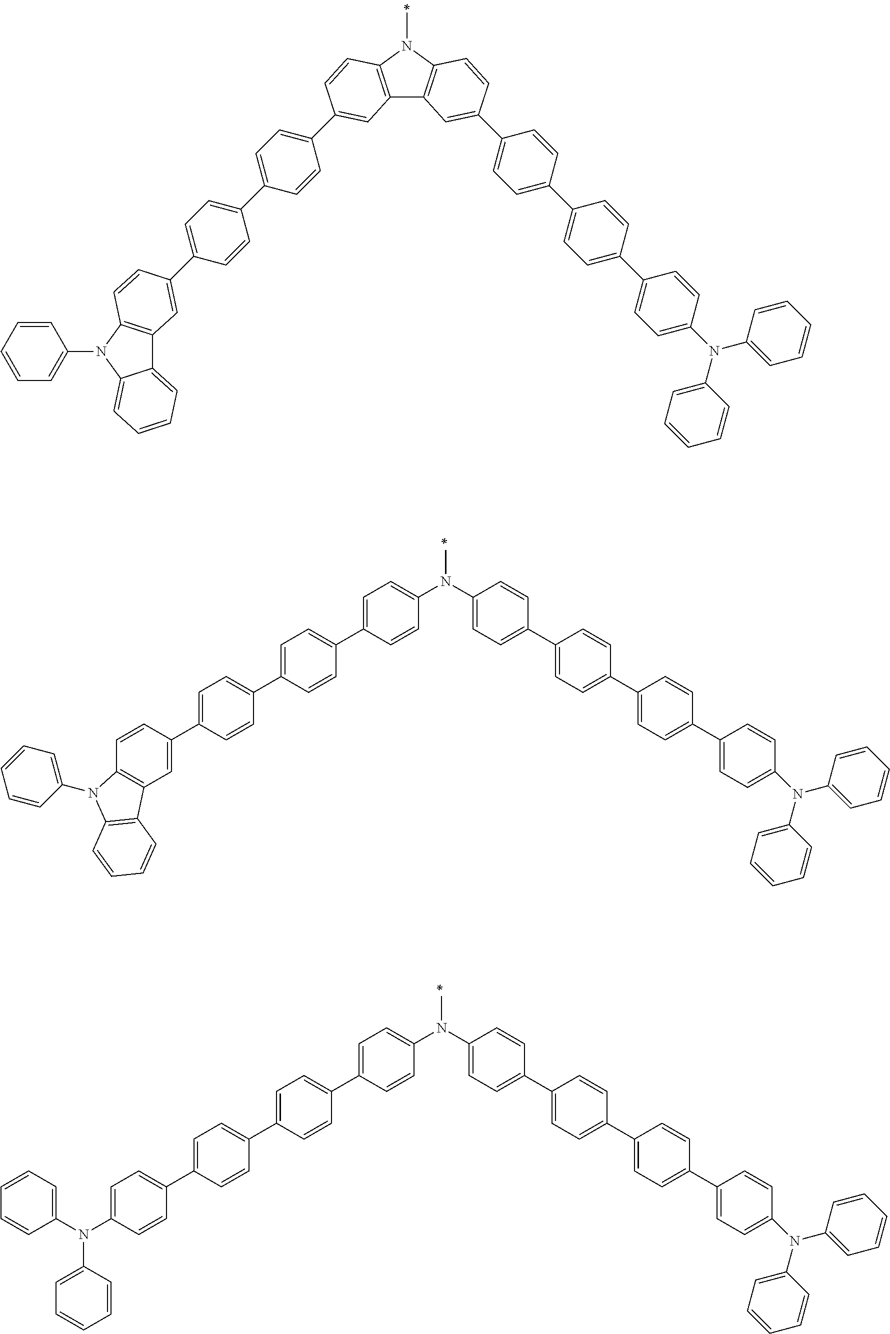 [SMILES: Cn1c2ccc(-c3ccc(-c4ccc(-c5ccc(N(c6ccccc6)c6ccccc6)cc5)cc4)cc3)cc2c2cc(-c3ccc(-c4ccc(-c5ccc6c(c5)c5ccccc5n6-c5ccccc5)cc4)cc3)ccc21 ; CN(c1ccc(-c2ccc(-c3ccc(-c4ccc(N(c5ccccc5)c5ccccc5)cc4)cc3)cc2)cc1)c1ccc(-c2ccc(-c3ccc(-c4ccc5c(c4)c4ccccc4n5-c4ccccc4)cc3)cc2)cc1 ; CN(c1ccc(-c2ccc(-c3ccc(-c4ccc(N(c5ccccc5)c5ccccc5)cc4)cc3)cc2)cc1)c1ccc(-c2ccc(-c3ccc(-c4ccc(N(c5ccccc5)c5ccccc5)cc4)cc3)cc2)cc1]

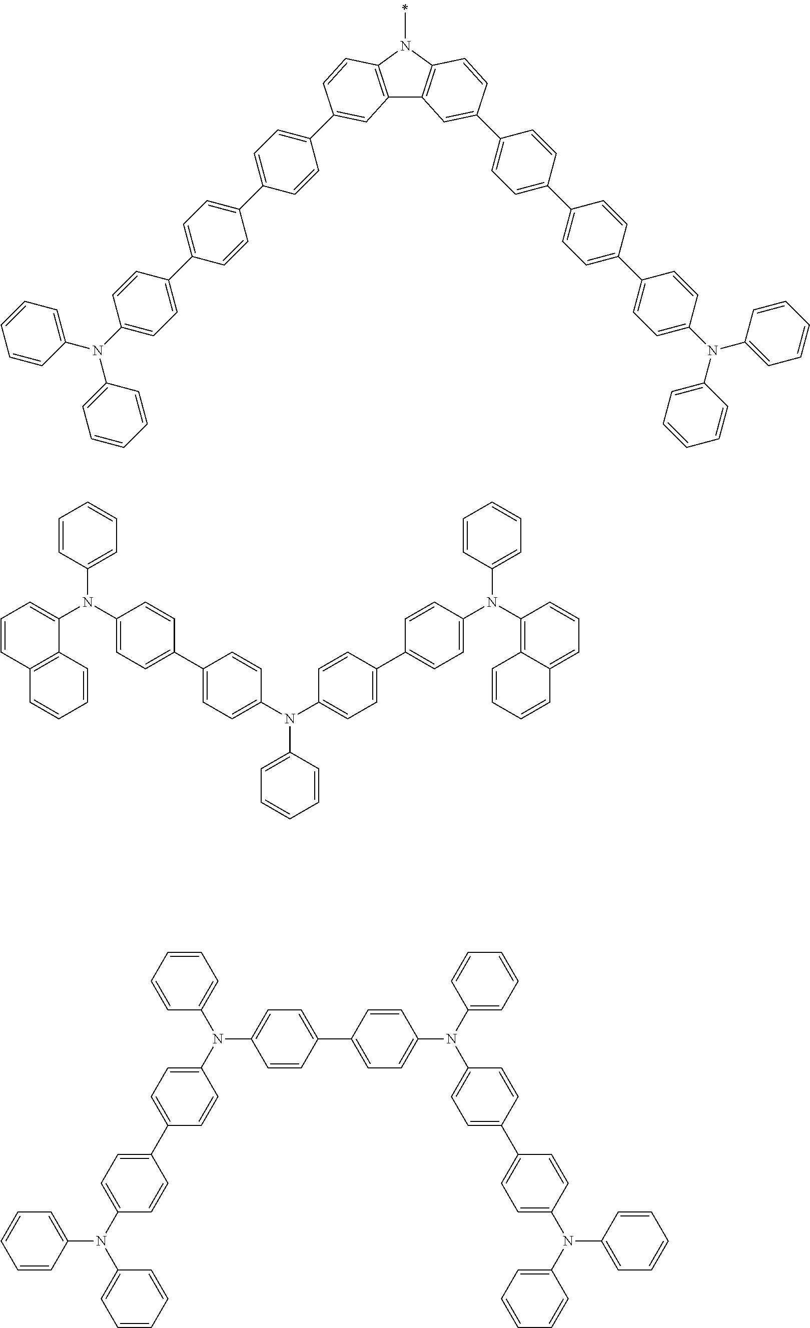 [SMILES: Cn1c2ccc(-c3ccc(-c4ccc(-c5ccc(N(c6ccccc6)c6ccccc6)cc5)cc4)cc3)cc2c2cc(-c3ccc(-c4ccc(-c5ccc(N(c6ccccc6)c6ccccc6)cc5)cc4)cc3)ccc21 ; c1ccc(N(c2ccc(-c3ccc(N(c4ccccc4)c4cccc5ccccc45)cc3)cc2)c2ccc(-c3ccc(N(c4ccccc4)c4cccc5ccccc45)cc3)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

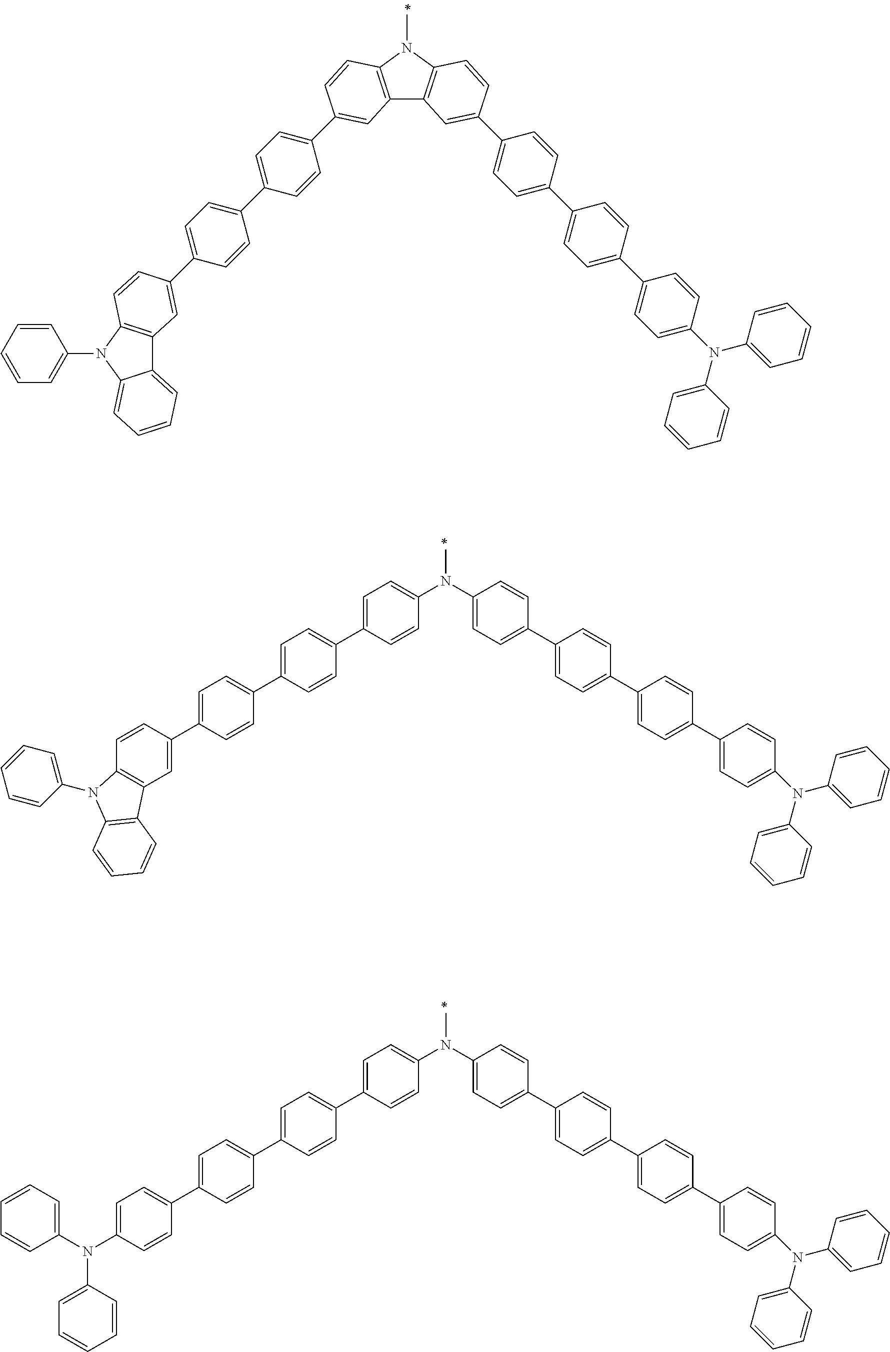 [SMILES: Cn1c2ccc(-c3ccc(-c4ccc(-c5ccc(N(c6ccccc6)c6ccccc6)cc5)cc4)cc3)cc2c2cc(-c3ccc(-c4ccc(-c5ccc6c(c5)c5ccccc5n6-c5ccccc5)cc4)cc3)ccc21 ; CN(c1ccc(-c2ccc(-c3ccc(-c4ccc(N(c5ccccc5)c5ccccc5)cc4)cc3)cc2)cc1)c1ccc(-c2ccc(-c3ccc(-c4ccc5c(c4)c4ccccc4n5-c4ccccc4)cc3)cc2)cc1 ; CN(c1ccc(-c2ccc(-c3ccc(-c4ccc(N(c5ccccc5)c5ccccc5)cc4)cc3)cc2)cc1)c1ccc(-c2ccc(-c3ccc(-c4ccc(N(c5ccccc5)c5ccccc5)cc4)cc3)cc2)cc1]

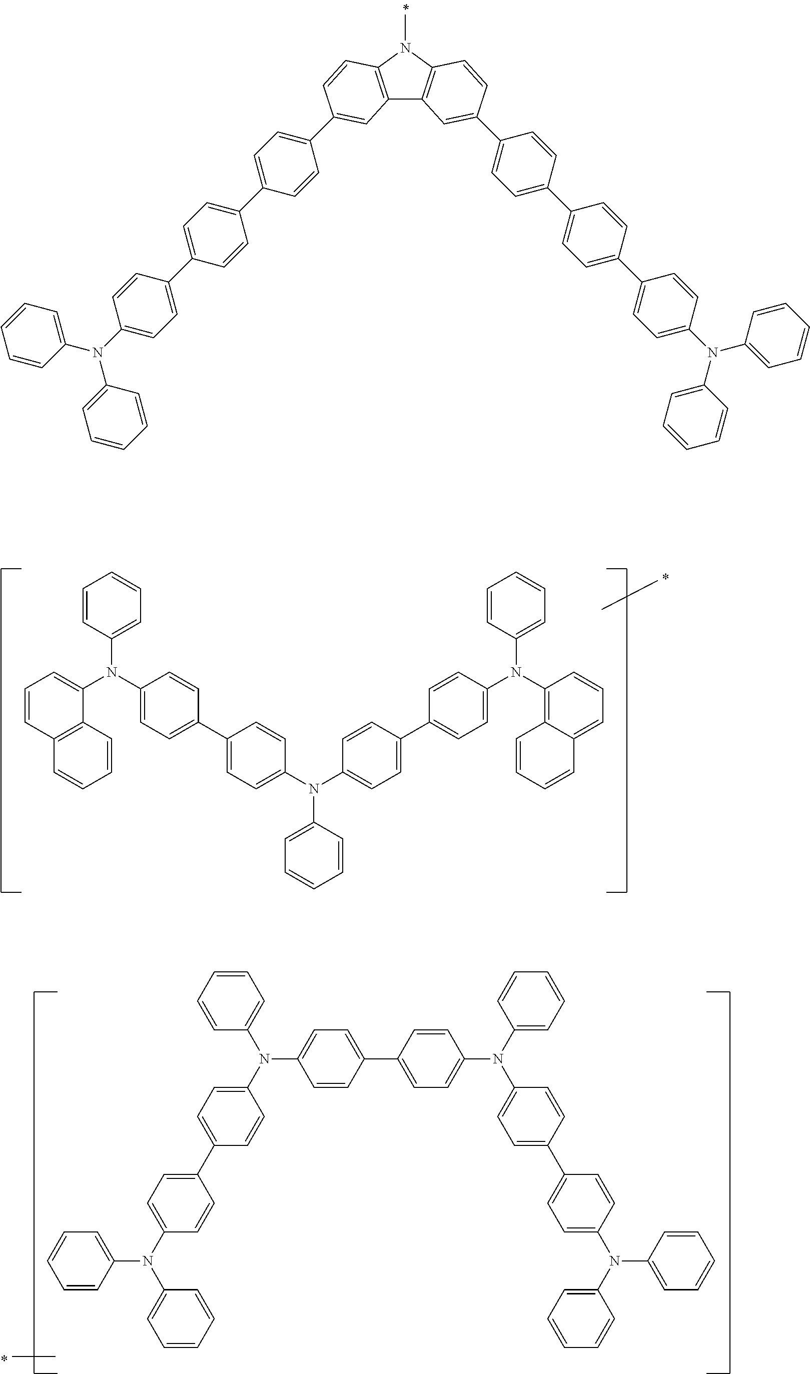

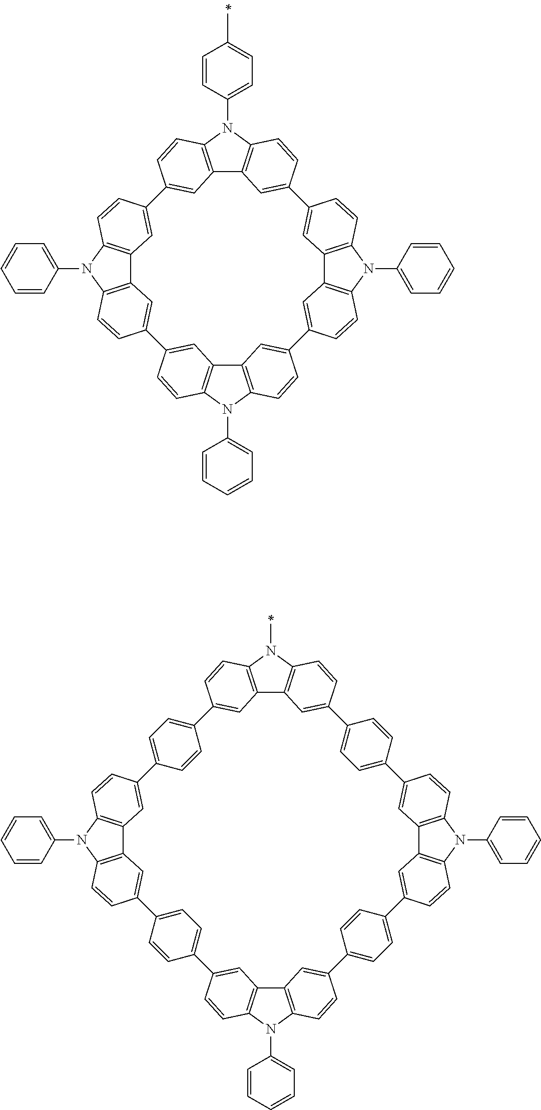

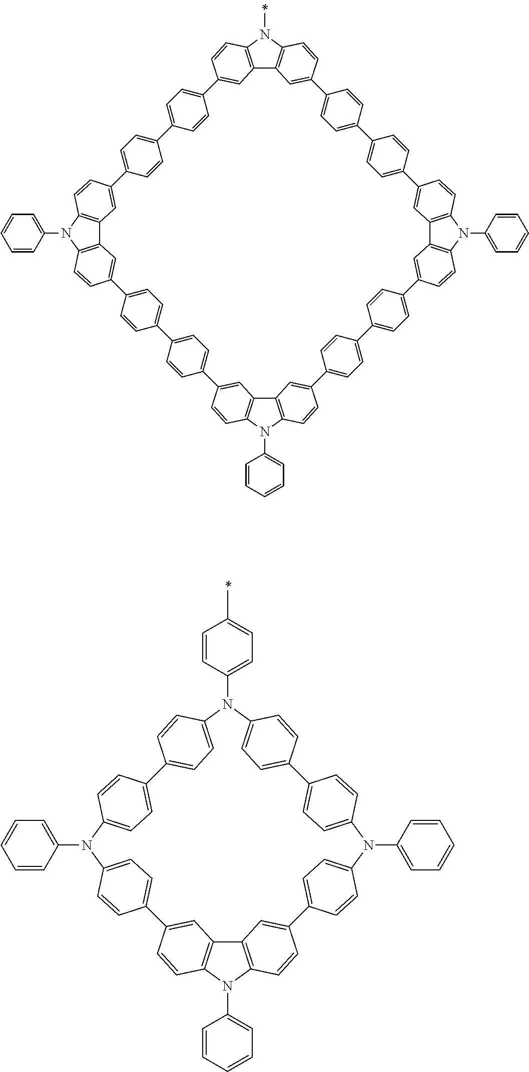

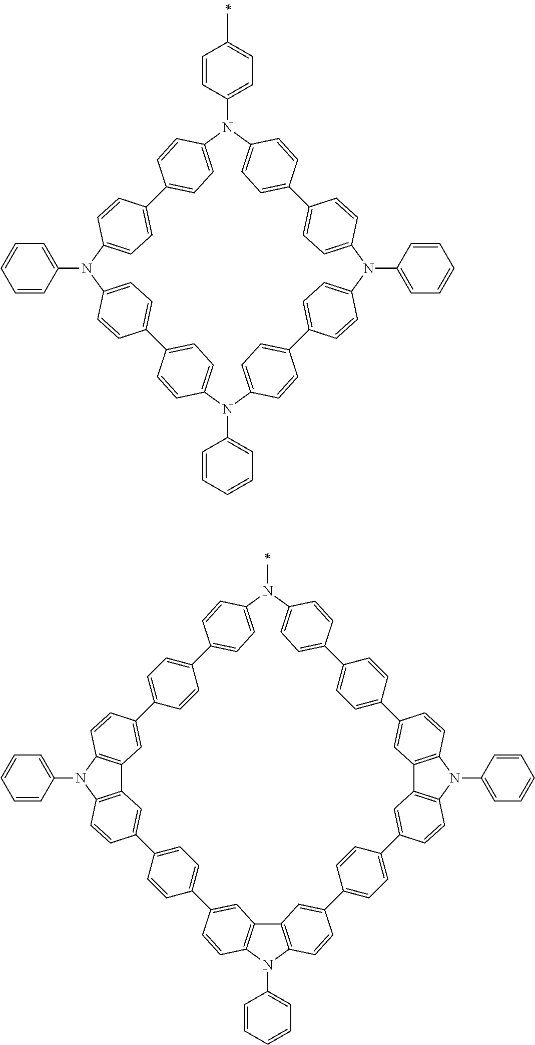

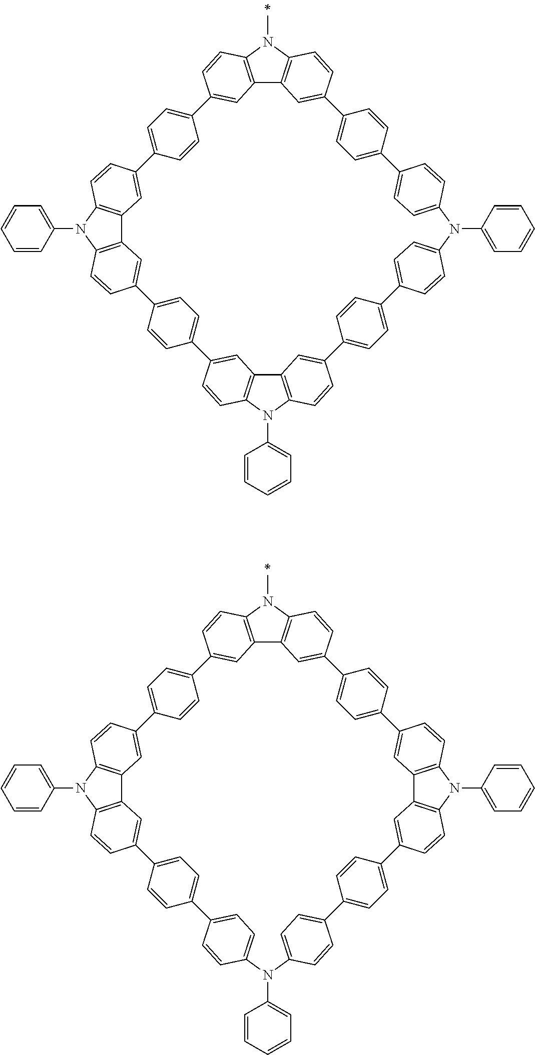 [SMILES: Cn1c2ccc3cc2c2cc(ccc21)-c1ccc(cc1)-c1ccc2c(c1)c1cc(ccc1n2-c1ccccc1)-c1ccc(cc1)-c1ccc2c(c1)c1cc(ccc1n2-c1ccccc1)-c1ccc(cc1)-c1ccc(cc1)N(c1ccccc1)c1ccc(cc1)-c1ccc-3cc1 ; Cn1c2ccc3cc2c2cc(ccc21)-c1ccc(cc1)-c1ccc2c(c1)c1cc(ccc1n2-c1ccccc1)-c1ccc(cc1)-c1ccc(cc1)N(c1ccccc1)c1ccc(cc1)-c1ccc(cc1)-c1ccc2c(c1)c1cc(ccc1n2-c1ccccc1)-c1ccc-3cc1]

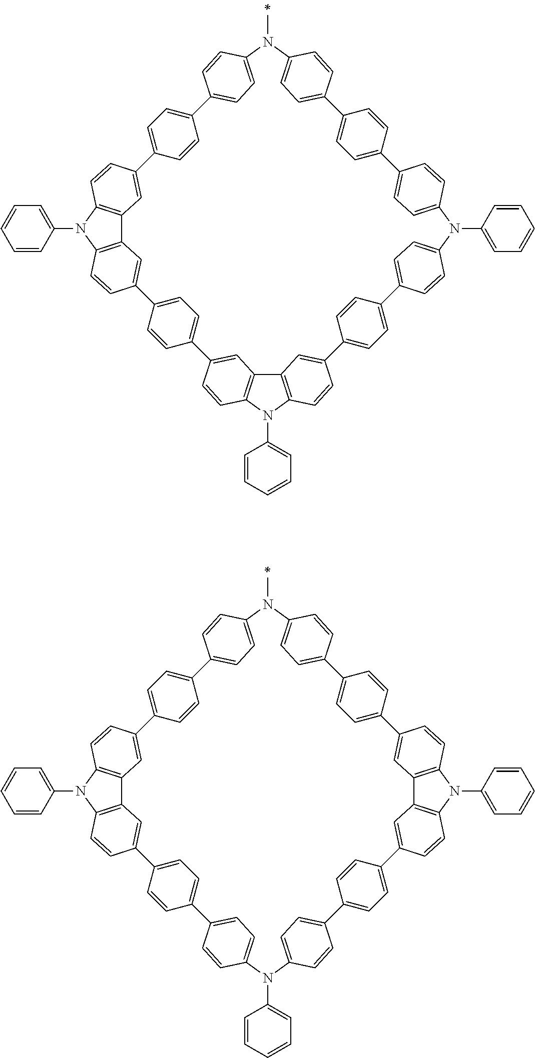 [SMILES: CN1c2ccc(cc2)-c2ccc(cc2)-c2ccc(cc2)N(c2ccccc2)c2ccc(cc2)-c2ccc(cc2)-c2ccc3c(c2)c2cc(ccc2n3-c2ccccc2)-c2ccc(cc2)-c2ccc3c(c2)c2cc(ccc2n3-c2ccccc2)-c2ccc(cc2)-c2ccc1cc2 ; CN1c2ccc(cc2)-c2ccc(cc2)-c2ccc3c(c2)c2cc(ccc2n3-c2ccccc2)-c2ccc(cc2)-c2ccc(cc2)N(c2ccccc2)c2ccc(cc2)-c2ccc(cc2)-c2ccc3c(c2)c2cc(ccc2n3-c2ccccc2)-c2ccc(cc2)-c2ccc1cc2]

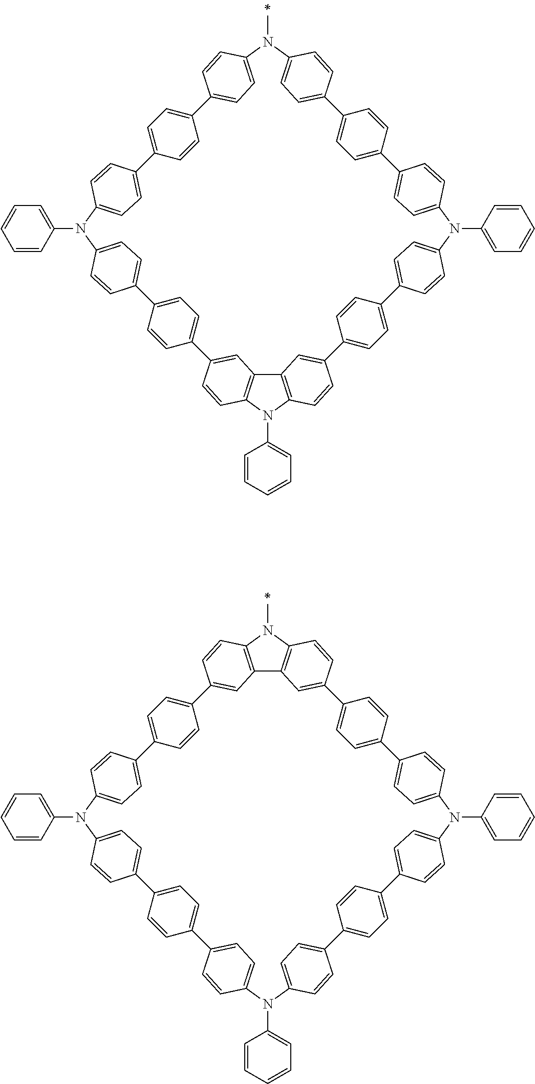 [SMILES: CN1c2ccc(cc2)-c2ccc(cc2)-c2ccc(cc2)N(c2ccccc2)c2ccc(cc2)-c2ccc(cc2)-c2ccc3c(c2)c2cc(ccc2n3-c2ccccc2)-c2ccc(cc2)-c2ccc(cc2)N(c2ccccc2)c2ccc(cc2)-c2ccc(cc2)-c2ccc1cc2 ; Cn1c2ccc3cc2c2cc(ccc21)-c1ccc(cc1)-c1ccc(cc1)N(c1ccccc1)c1ccc(cc1)-c1ccc(cc1)-c1ccc(cc1)N(c1ccccc1)c1ccc(cc1)-c1ccc(cc1)-c1ccc(cc1)N(c1ccccc1)c1ccc(cc1)-c1ccc-3cc1]

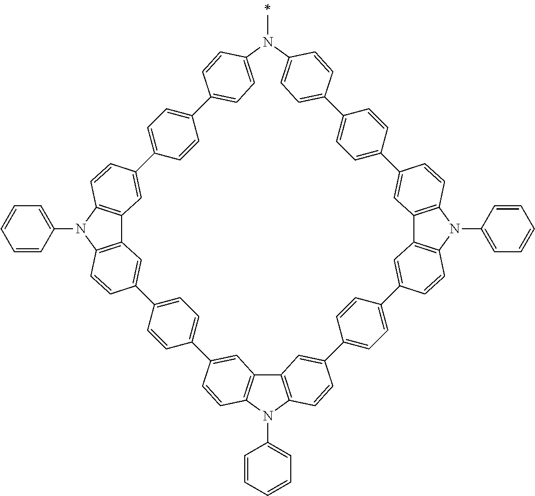 [SMILES: CN1c2ccc(cc2)-c2ccc(cc2)-c2ccc3c(c2)c2cc(ccc2n3-c2ccccc2)-c2ccc(cc2)-c2ccc3c(c2)c2cc(ccc2n3-c2ccccc2)-c2ccc(cc2)-c2ccc3c(c2)c2cc(ccc2n3-c2ccccc2)-c2ccc(cc2)-c2ccc1cc2]

Preferred structural units A are shown below.

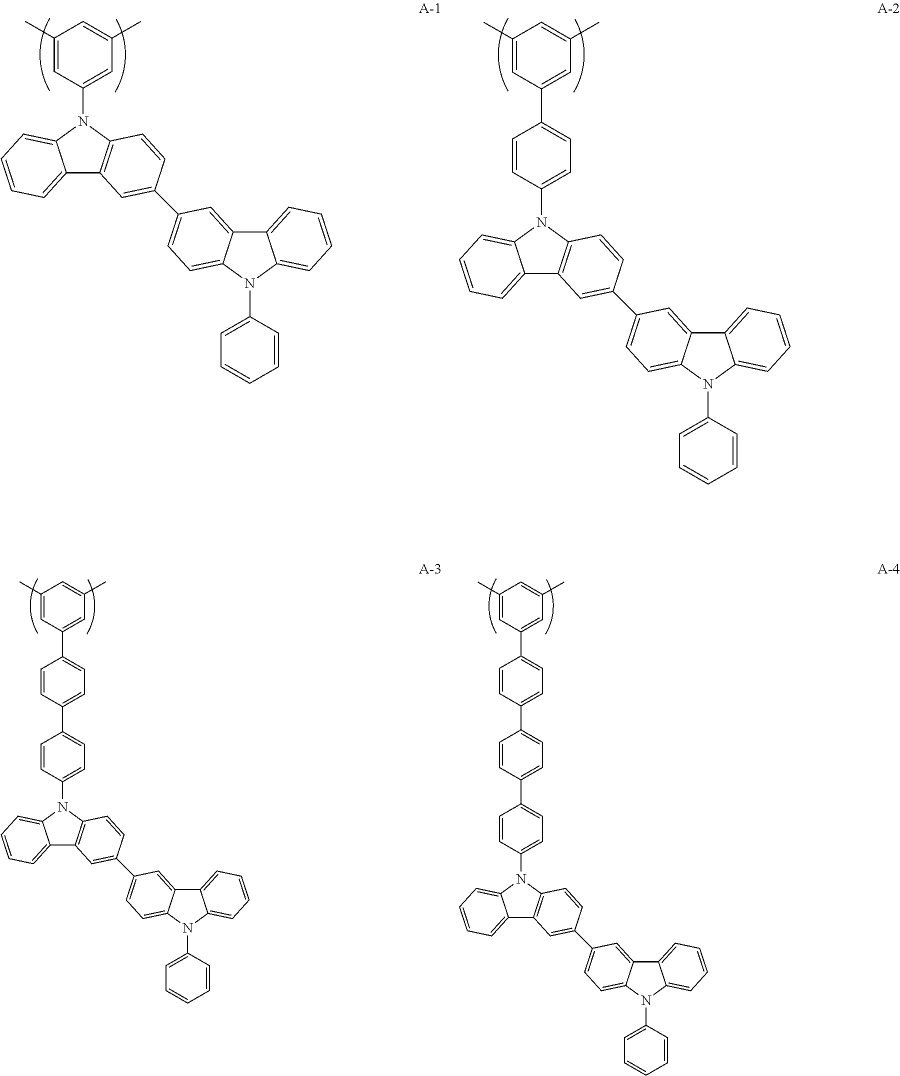

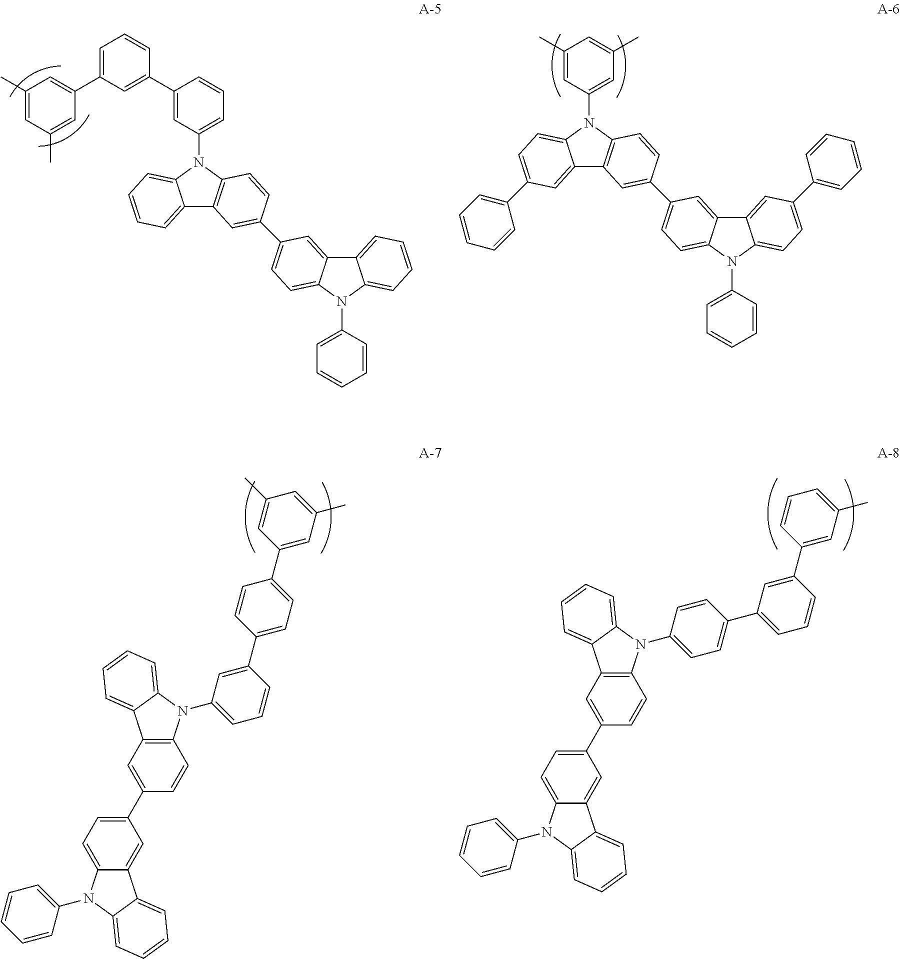

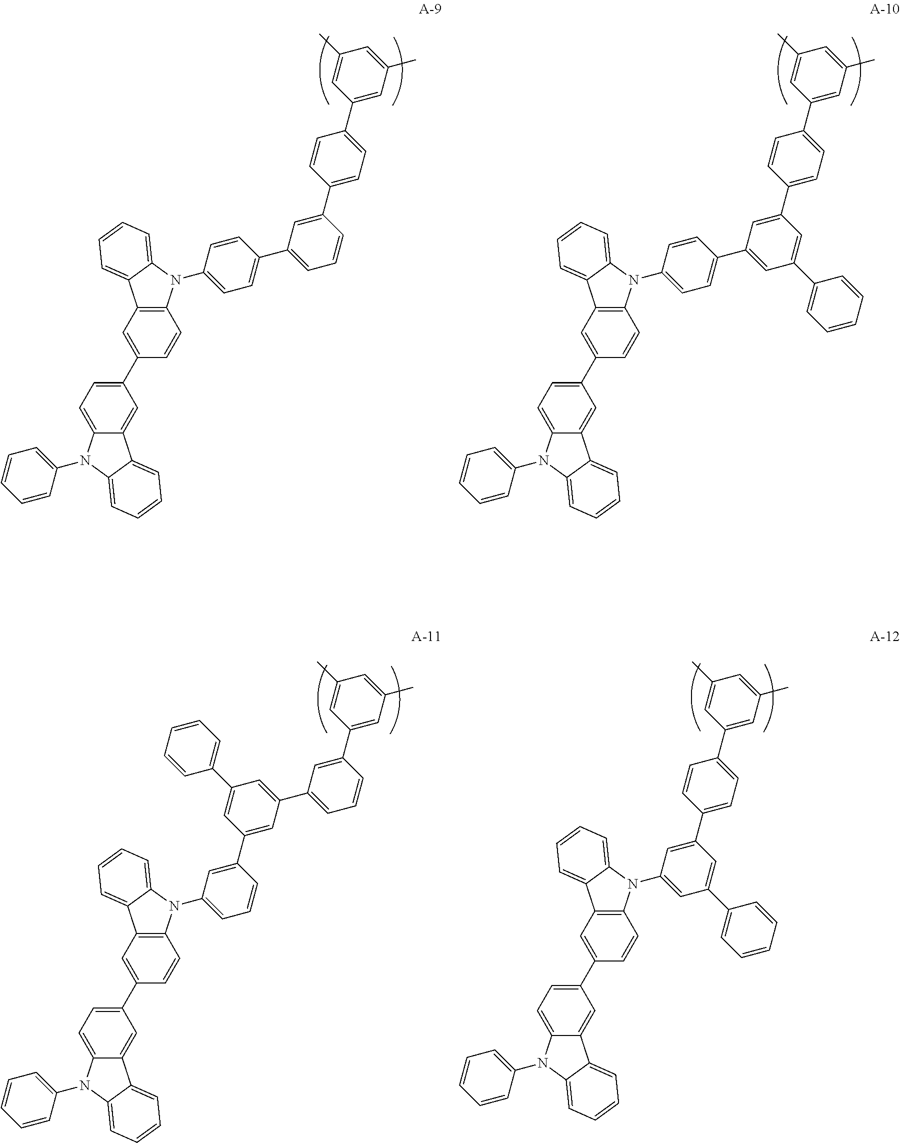

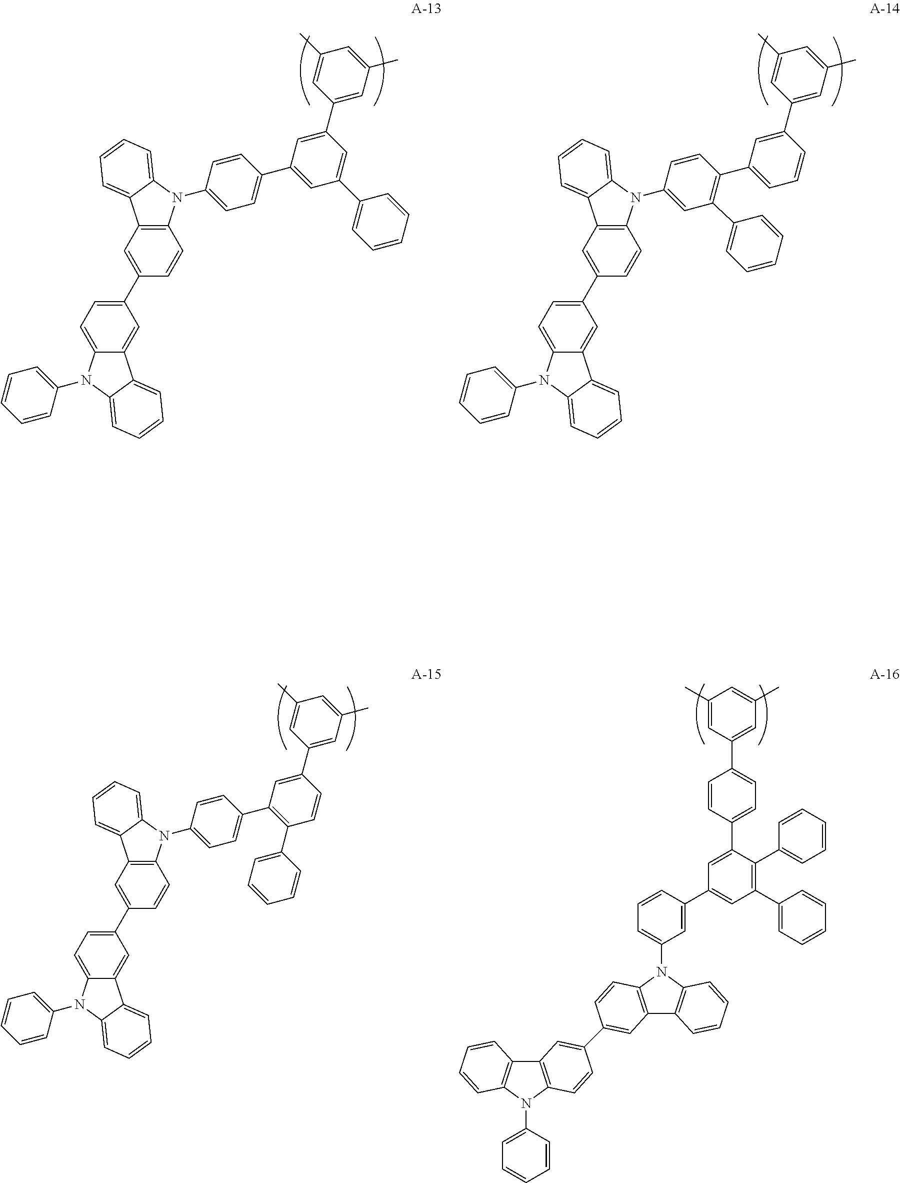

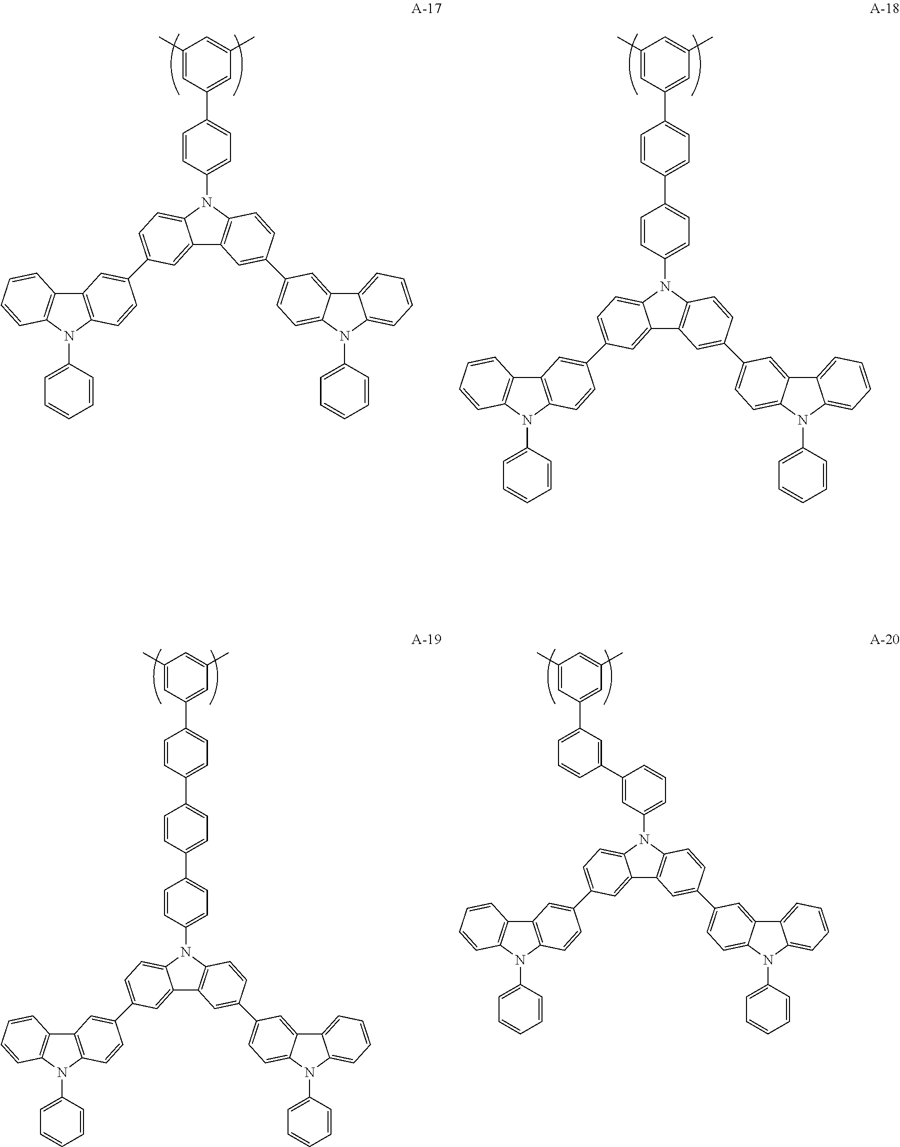

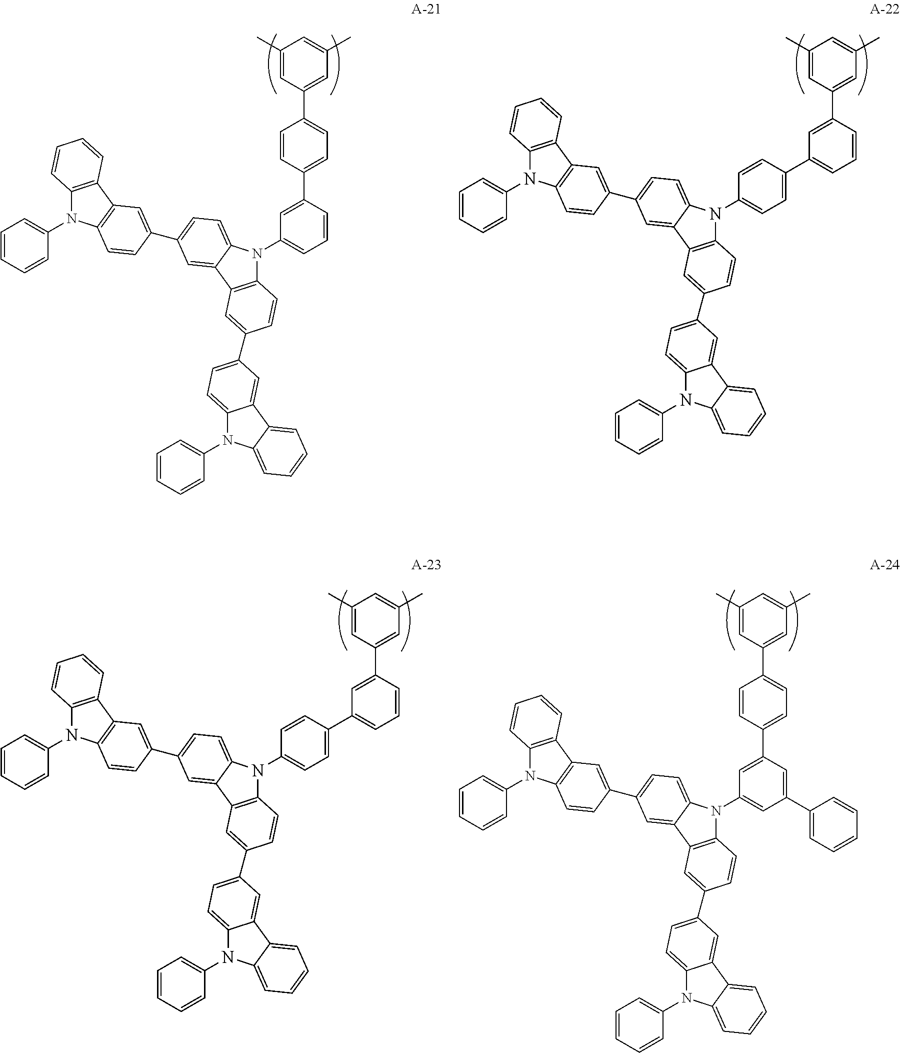

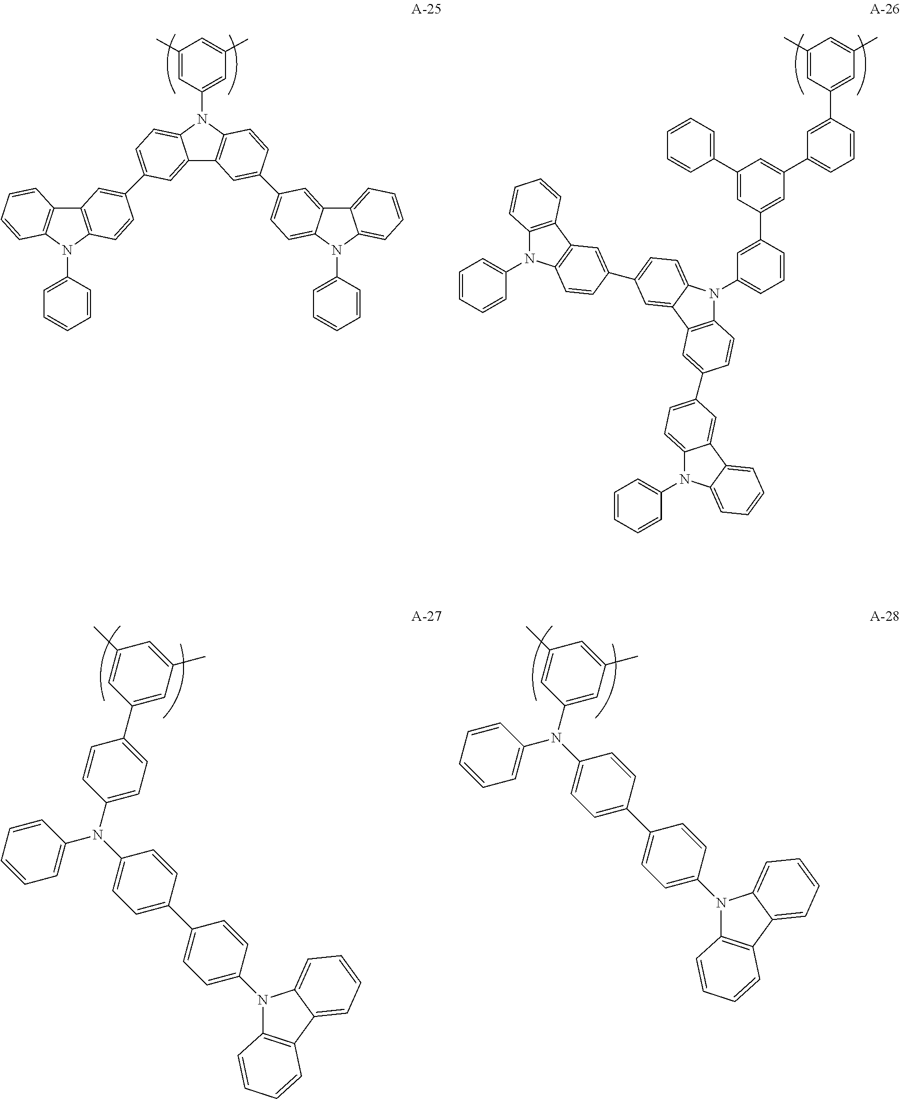

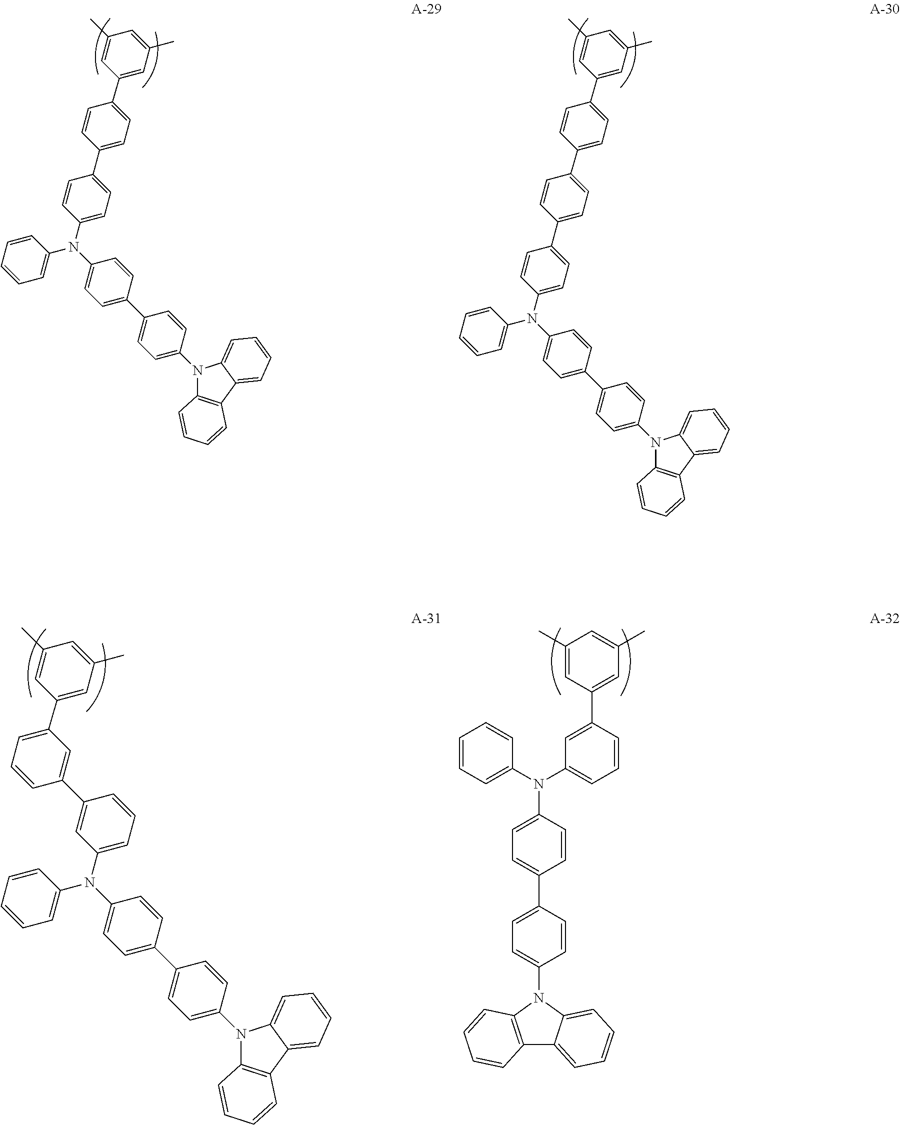

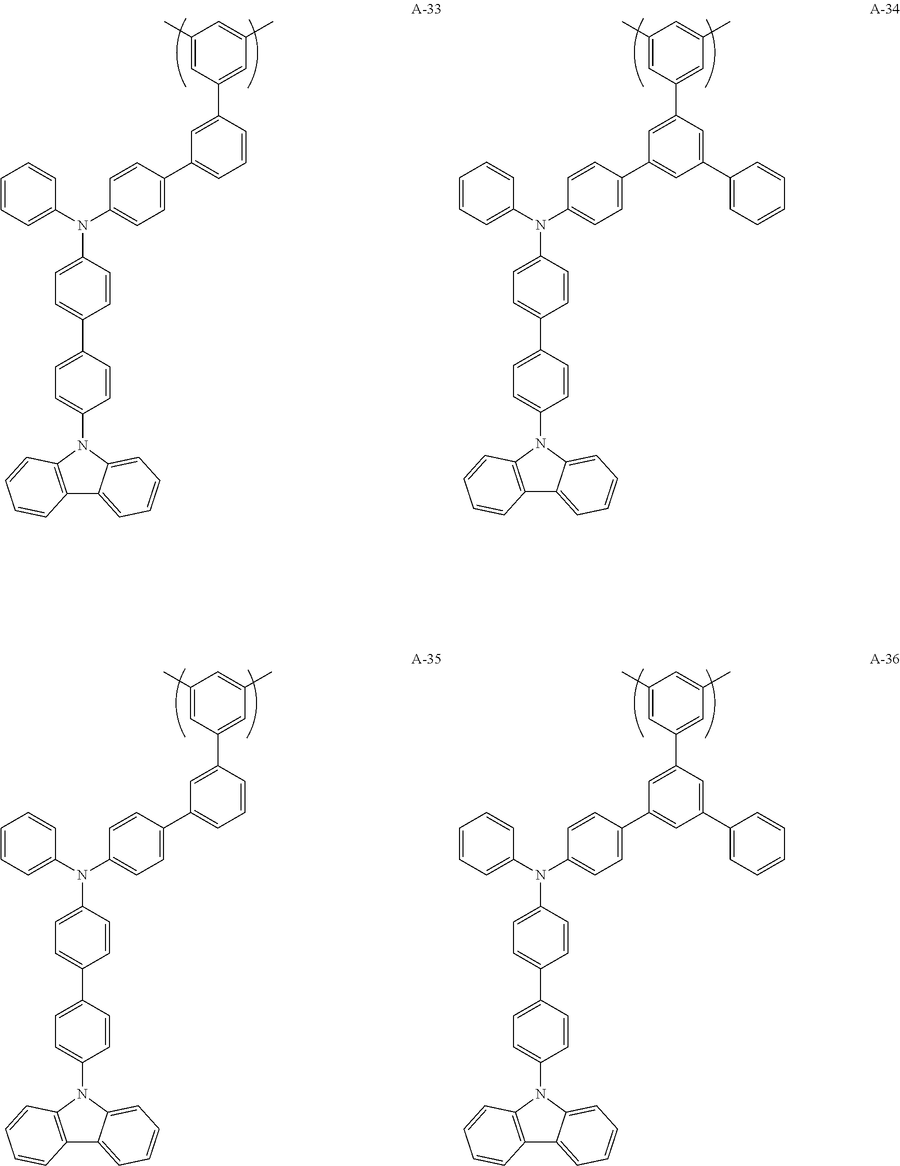

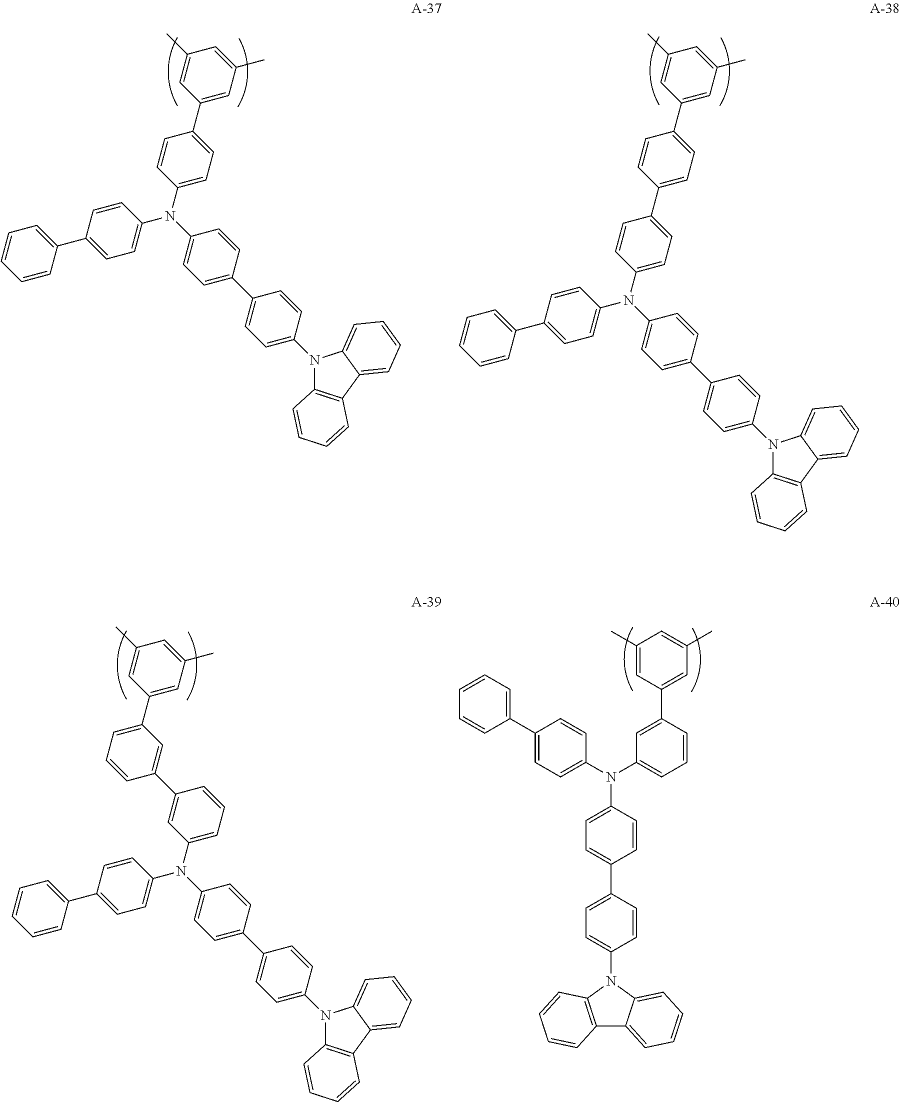

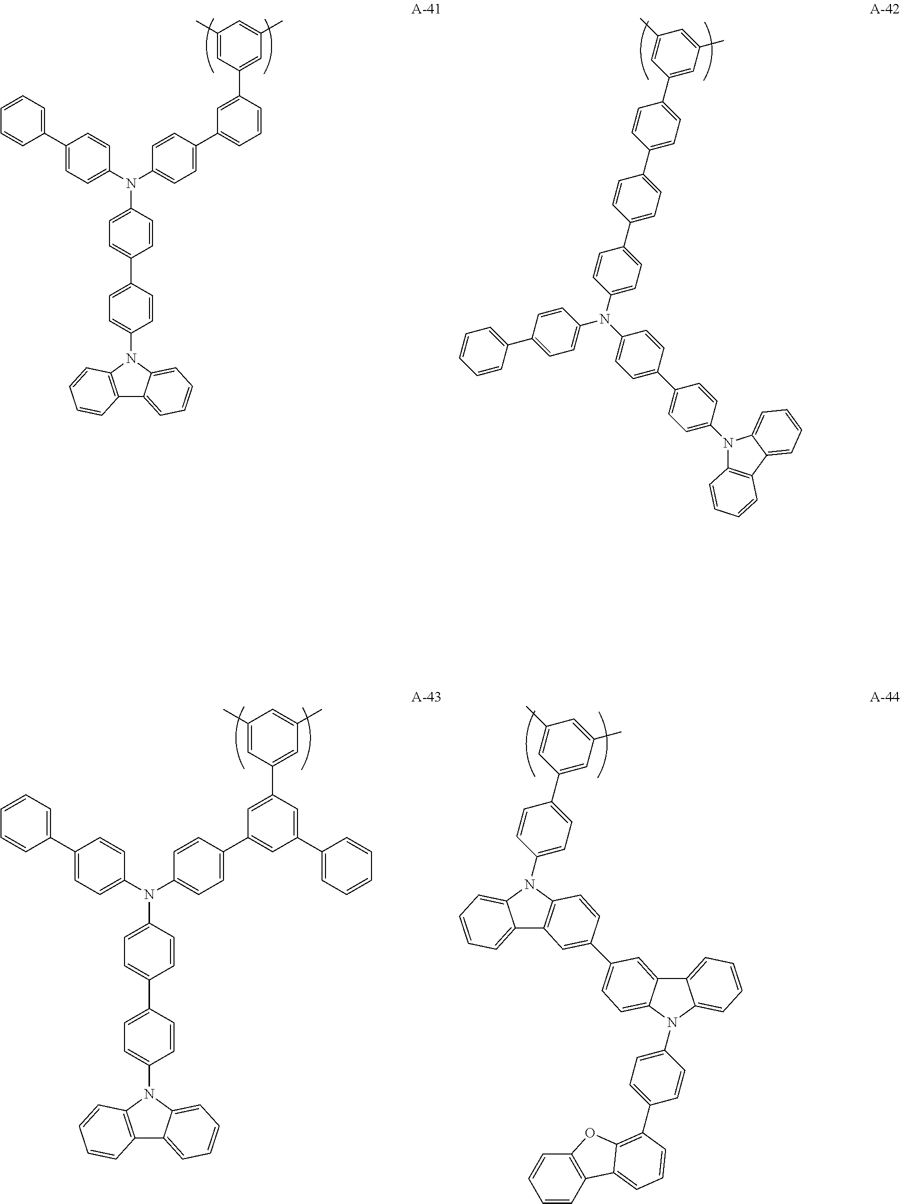

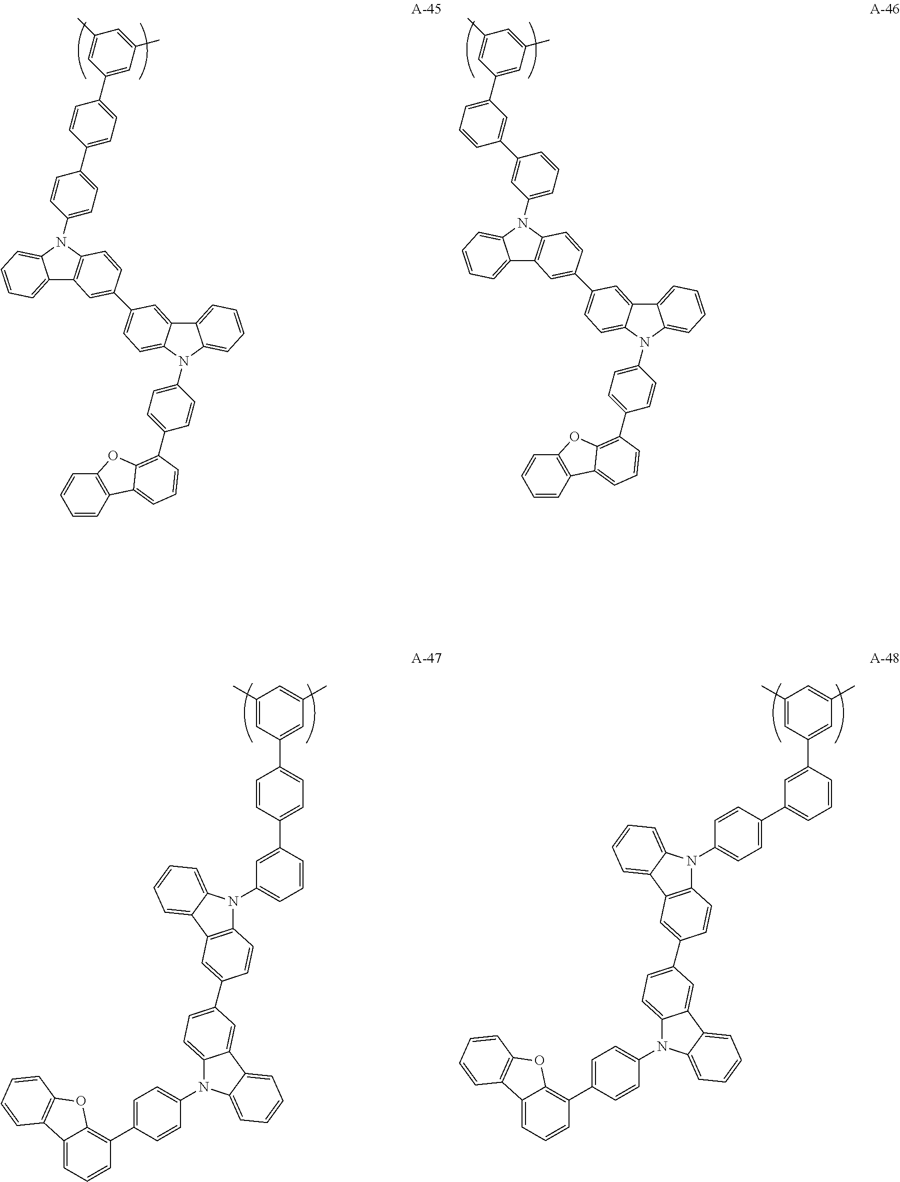

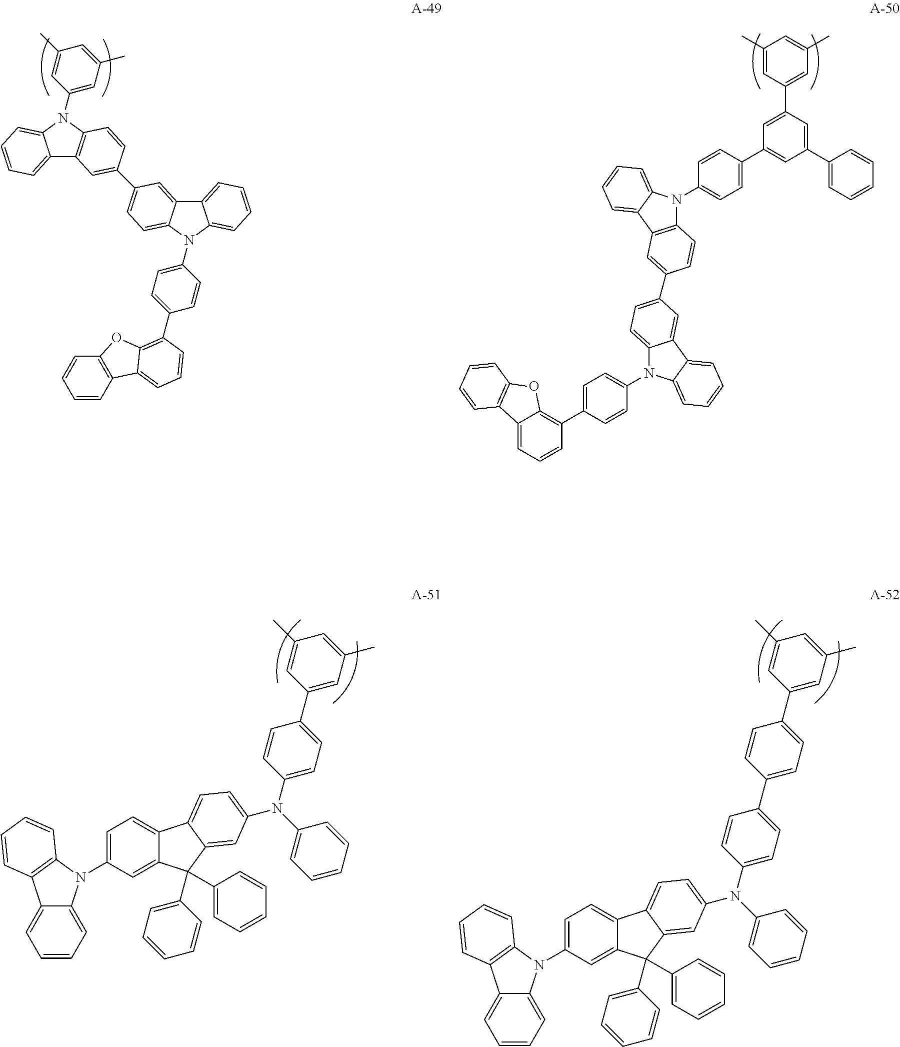

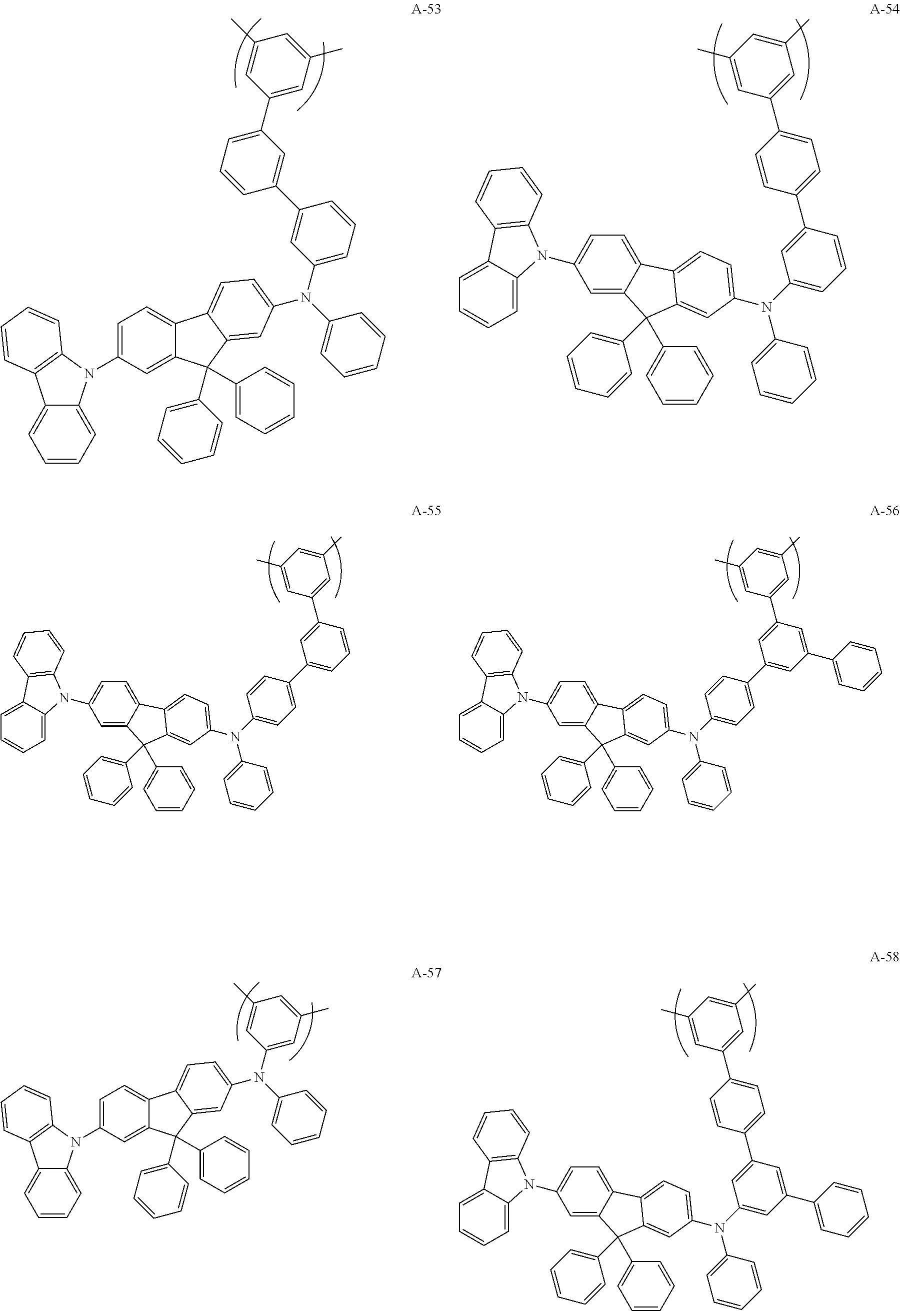

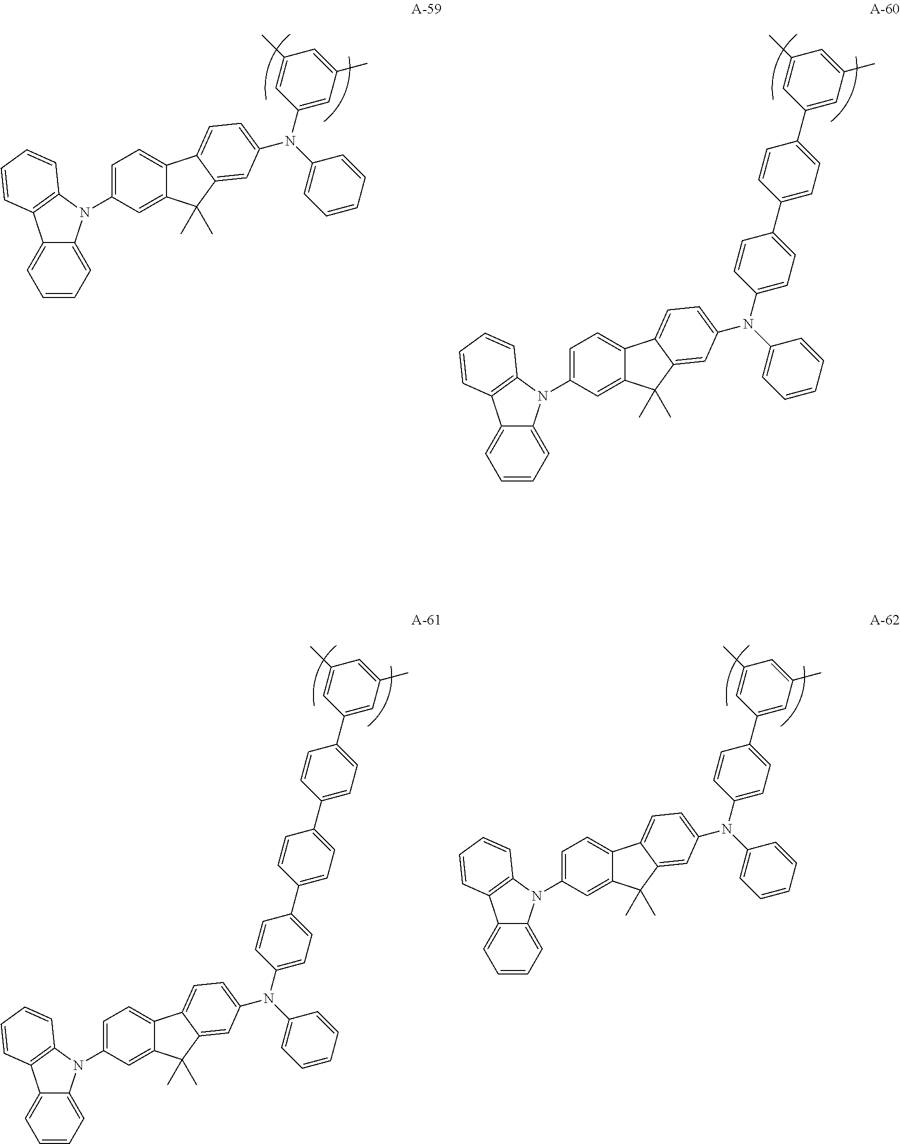

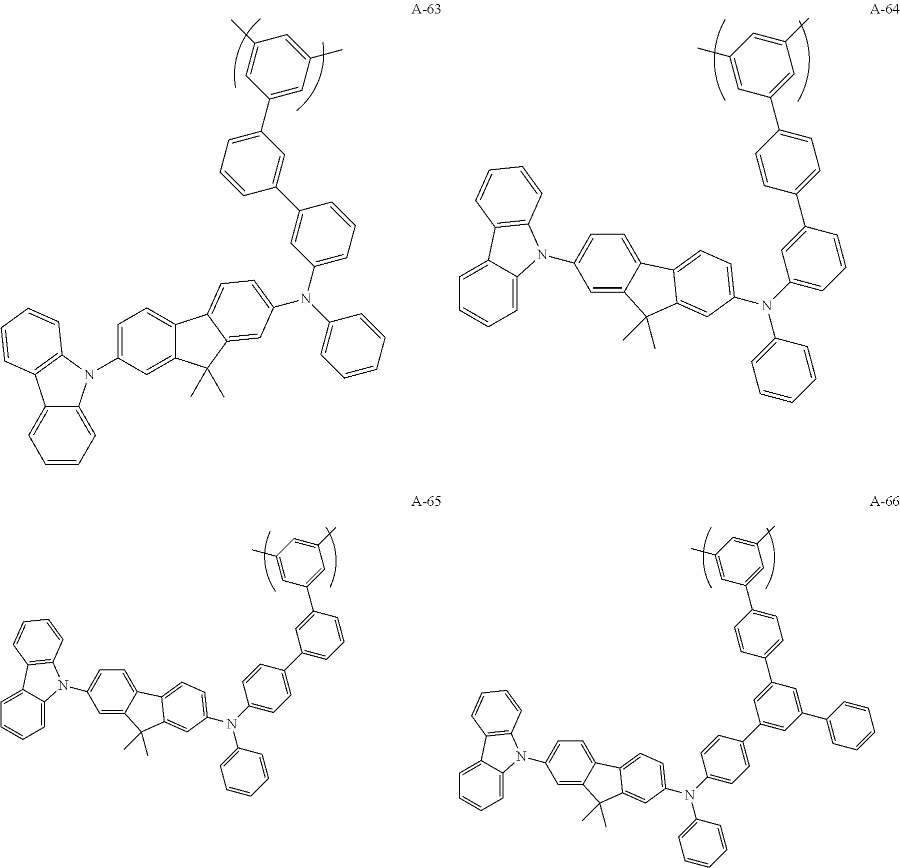

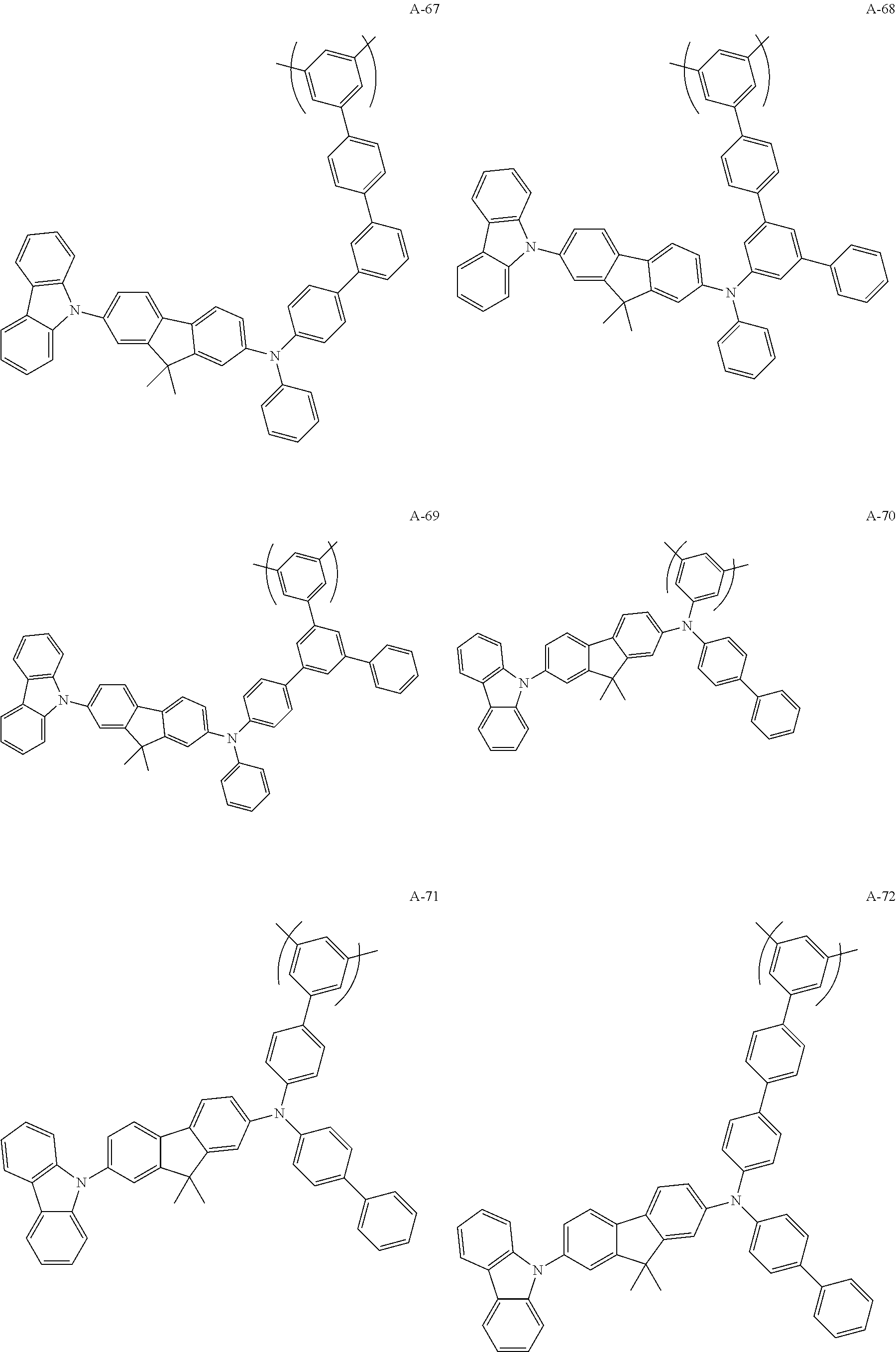

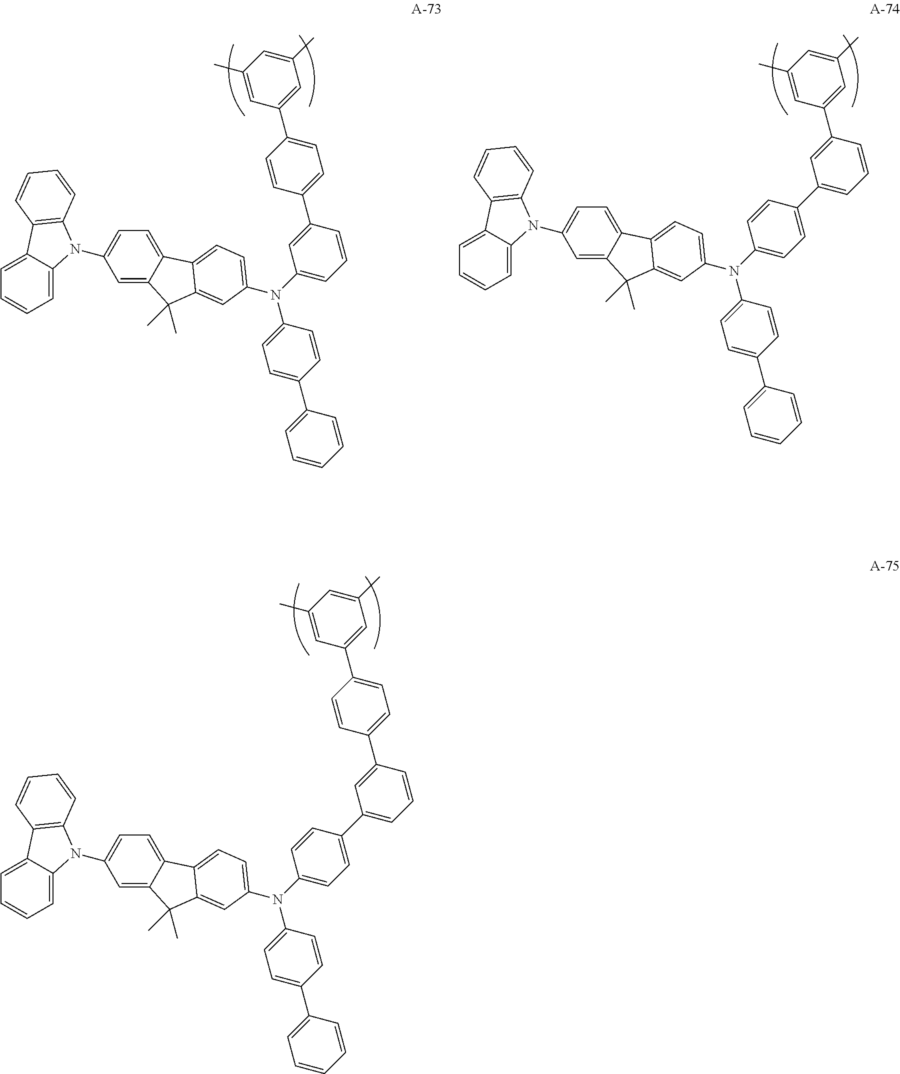

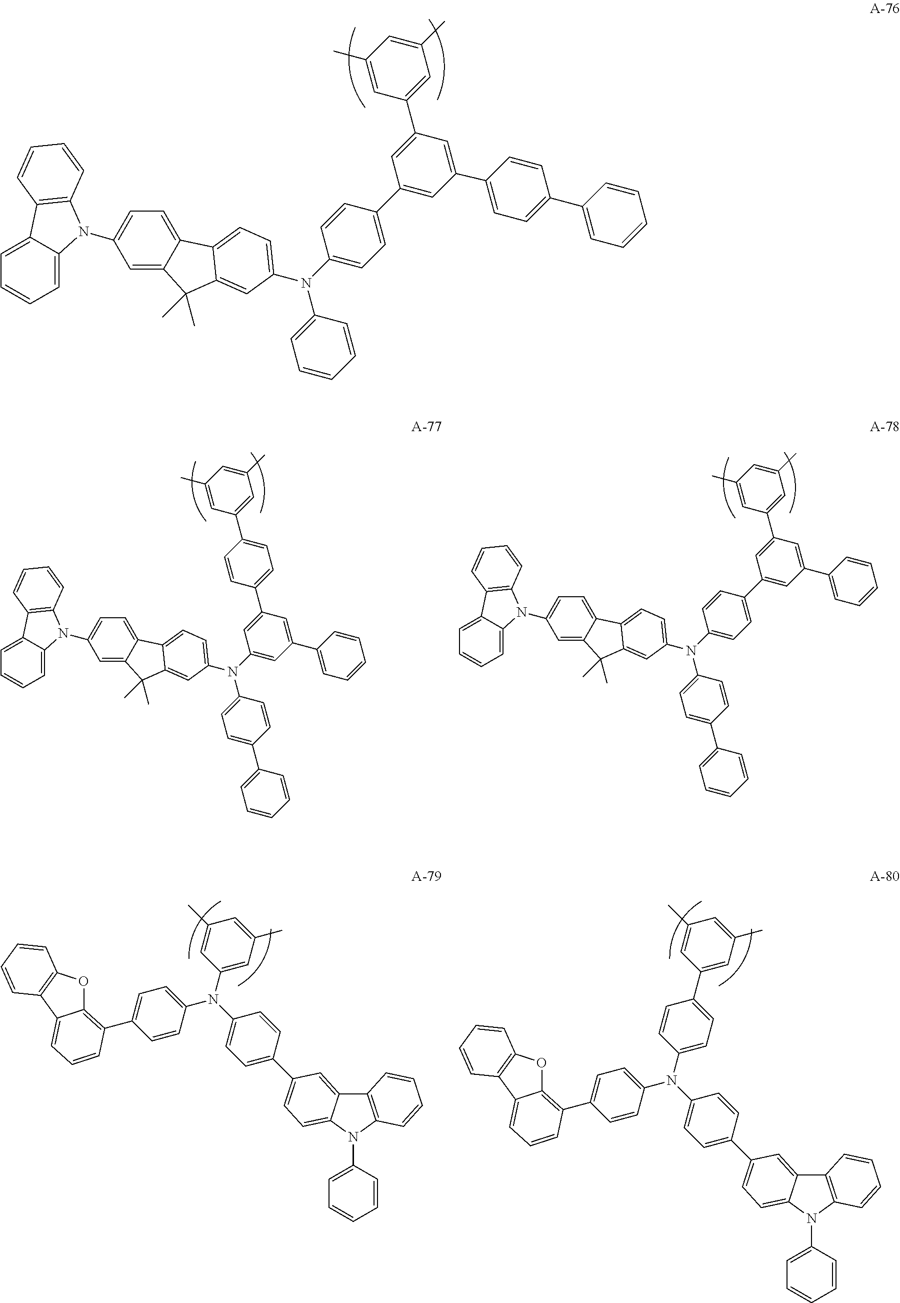

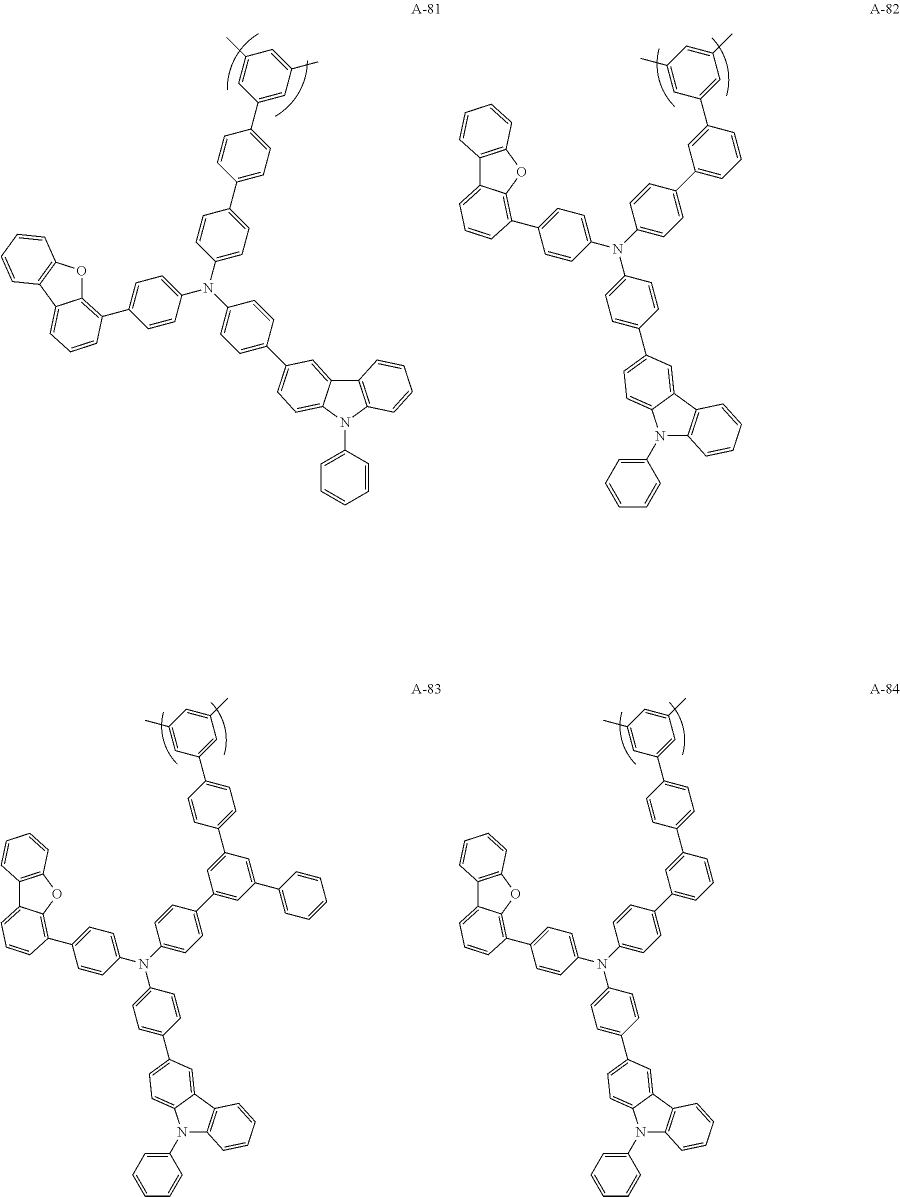

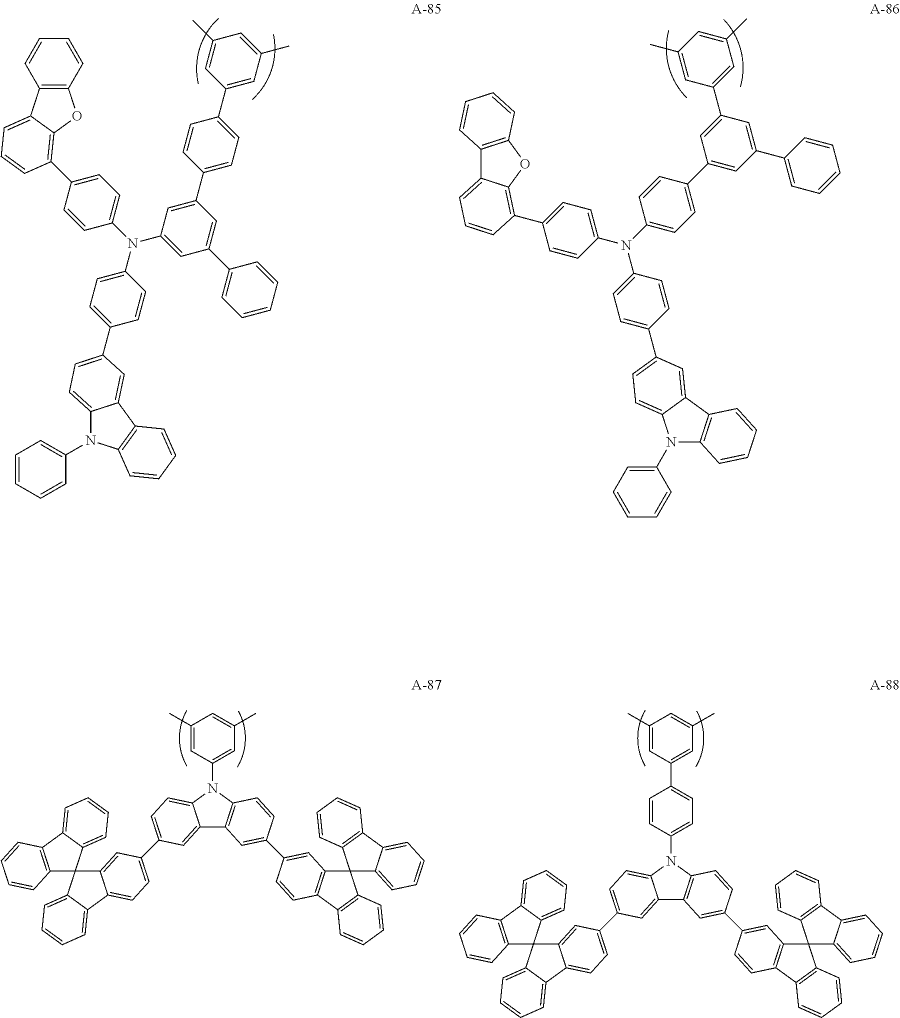

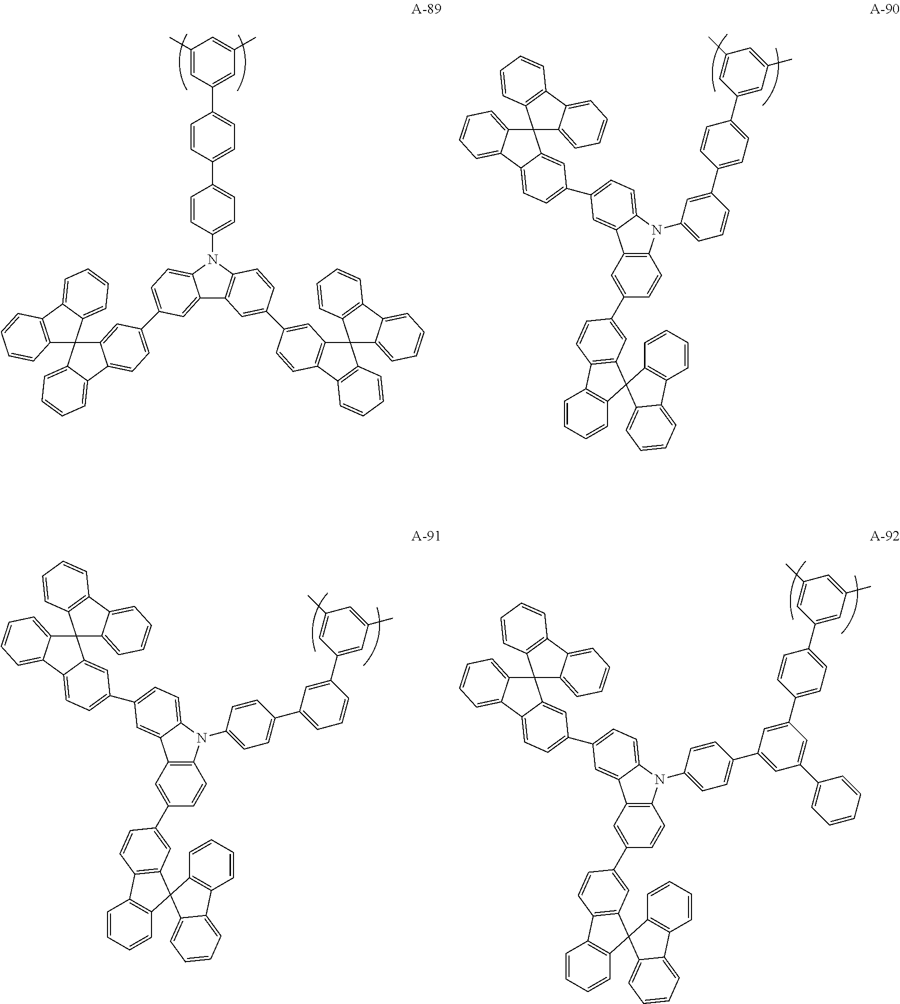

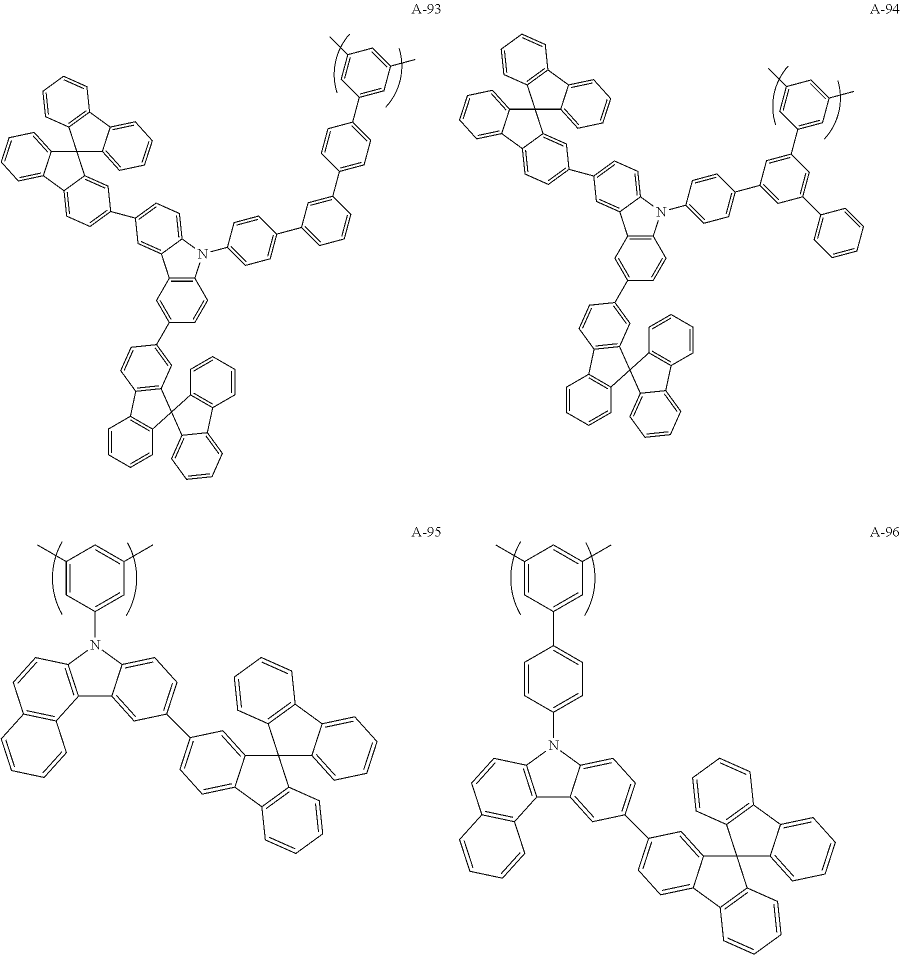

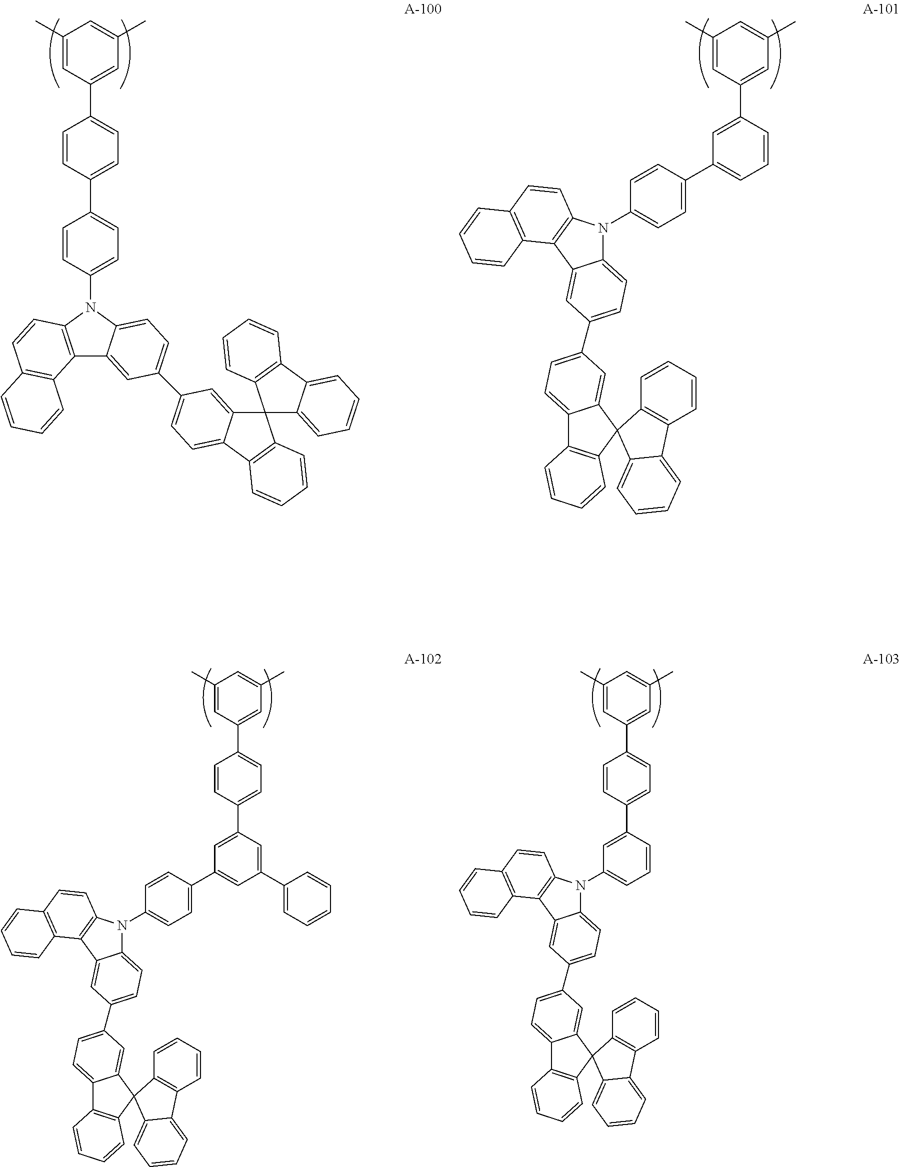

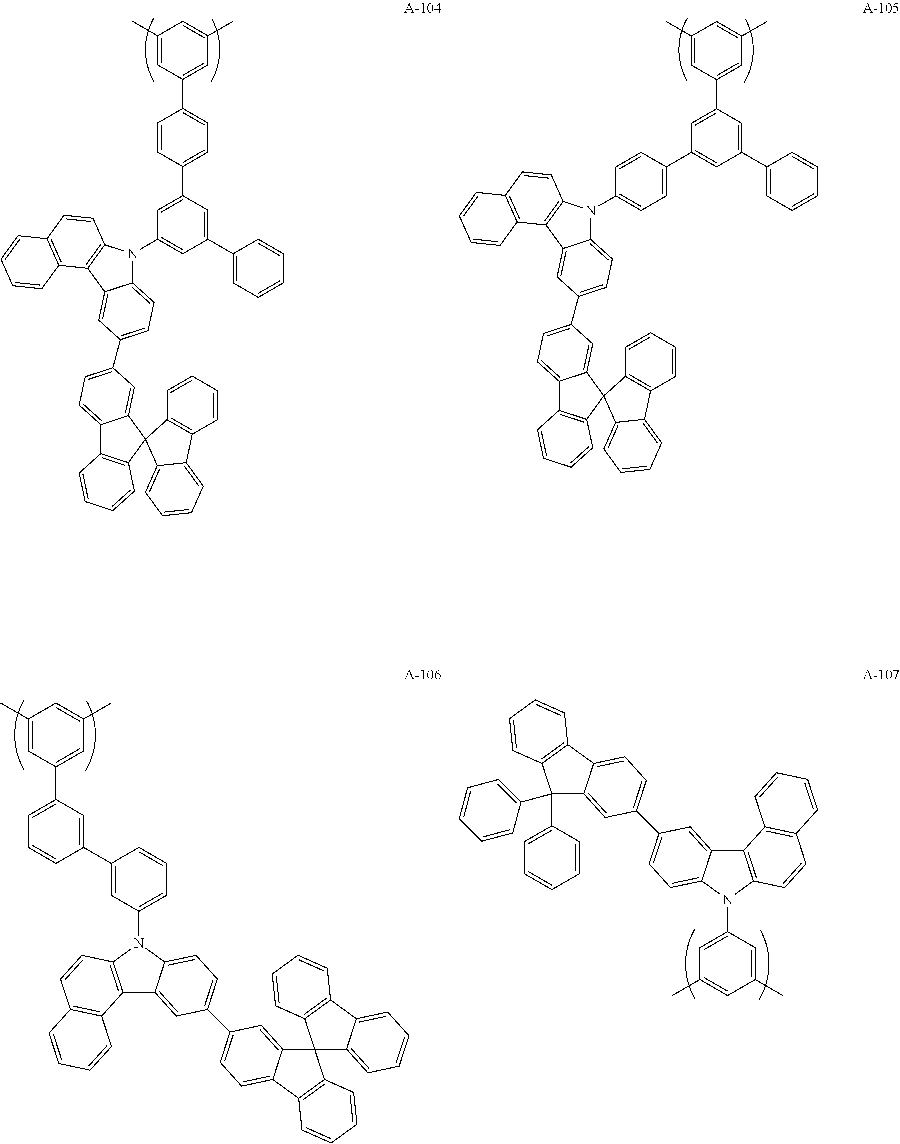

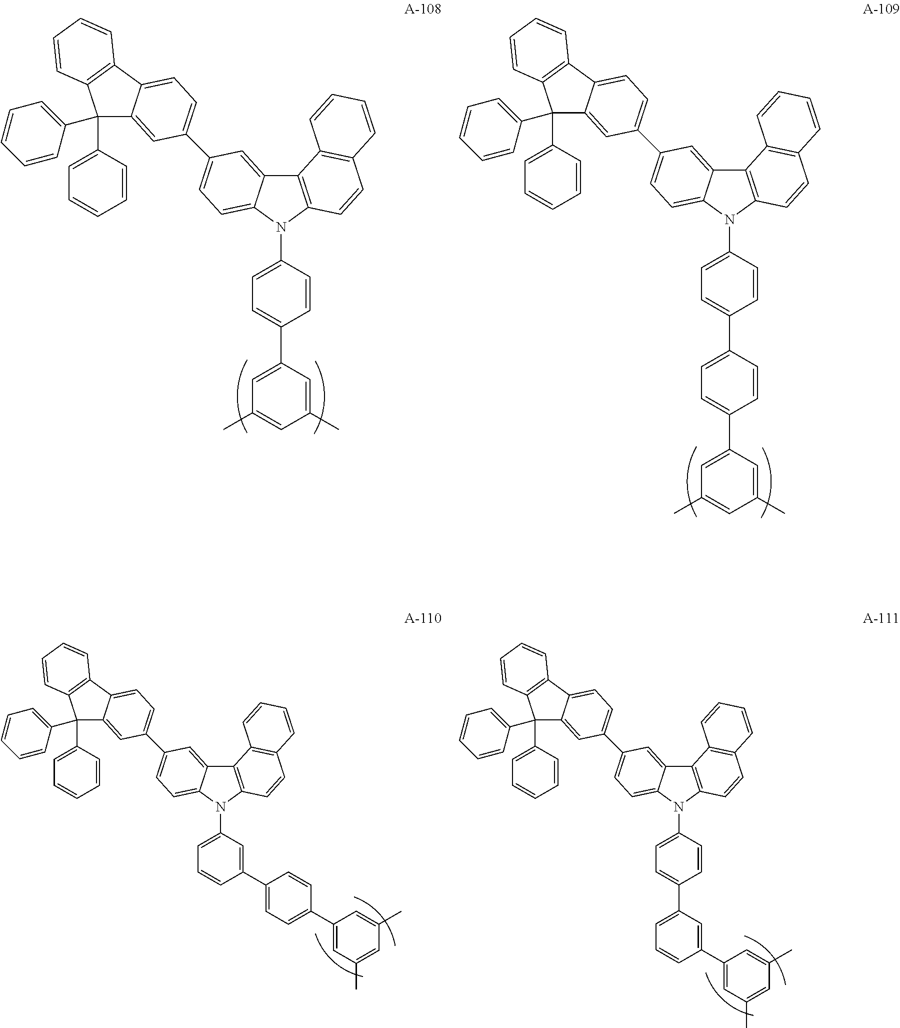

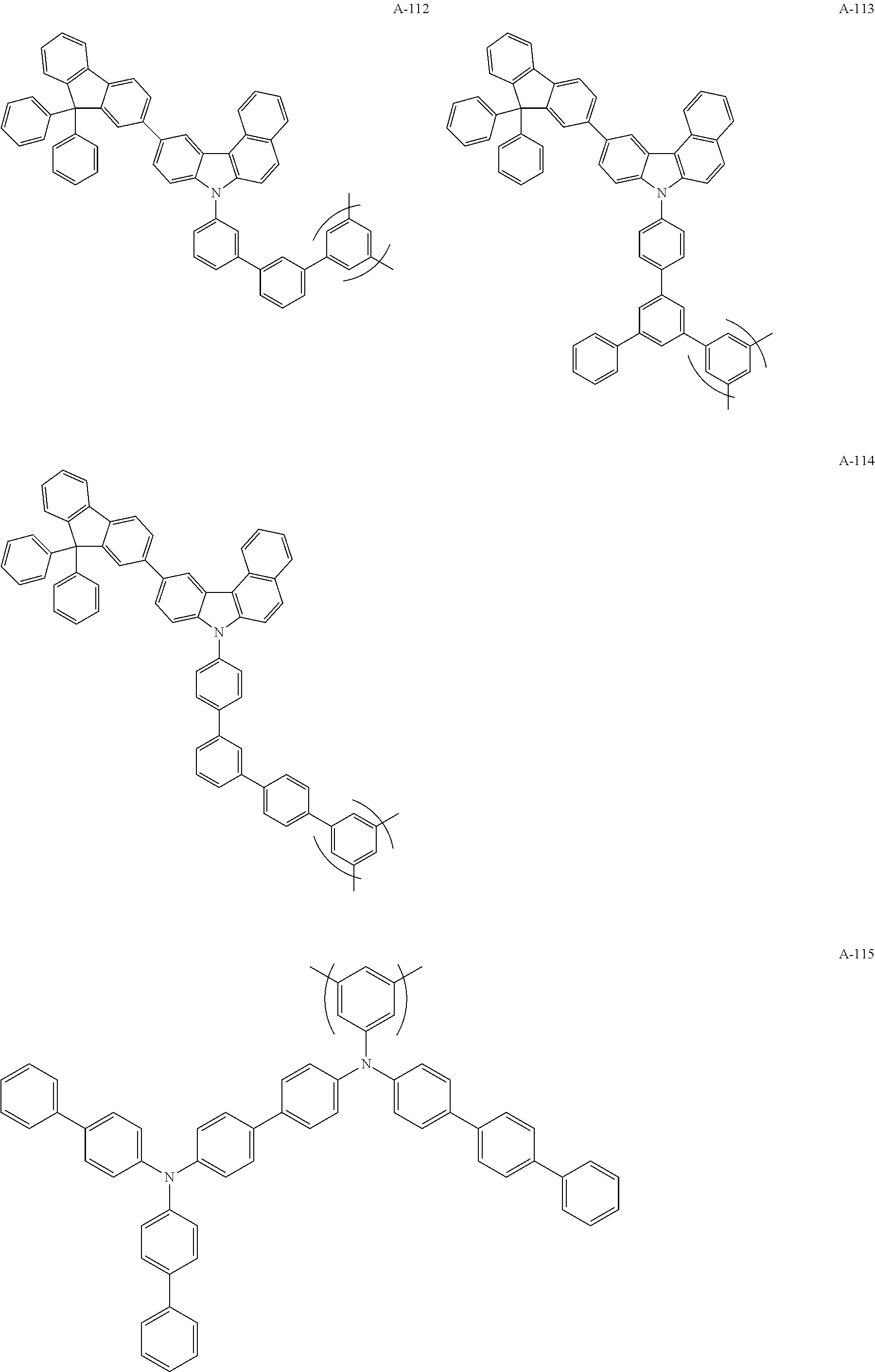

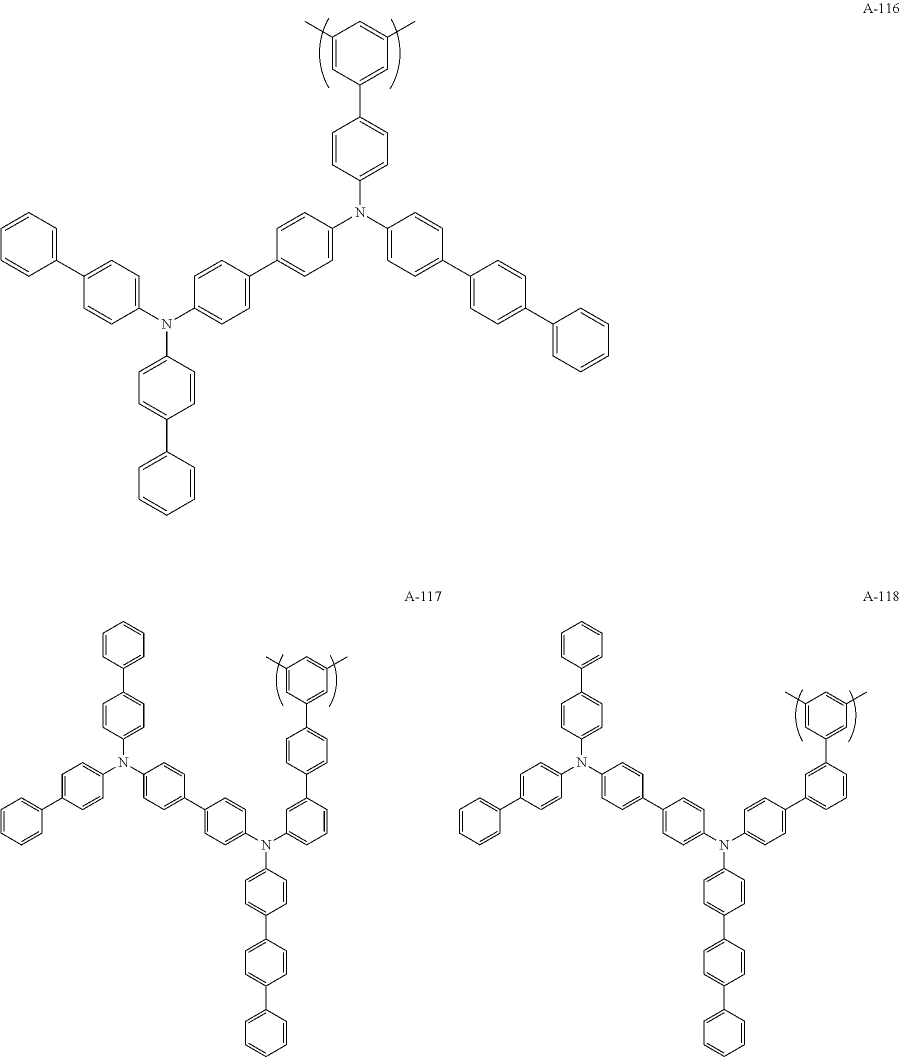

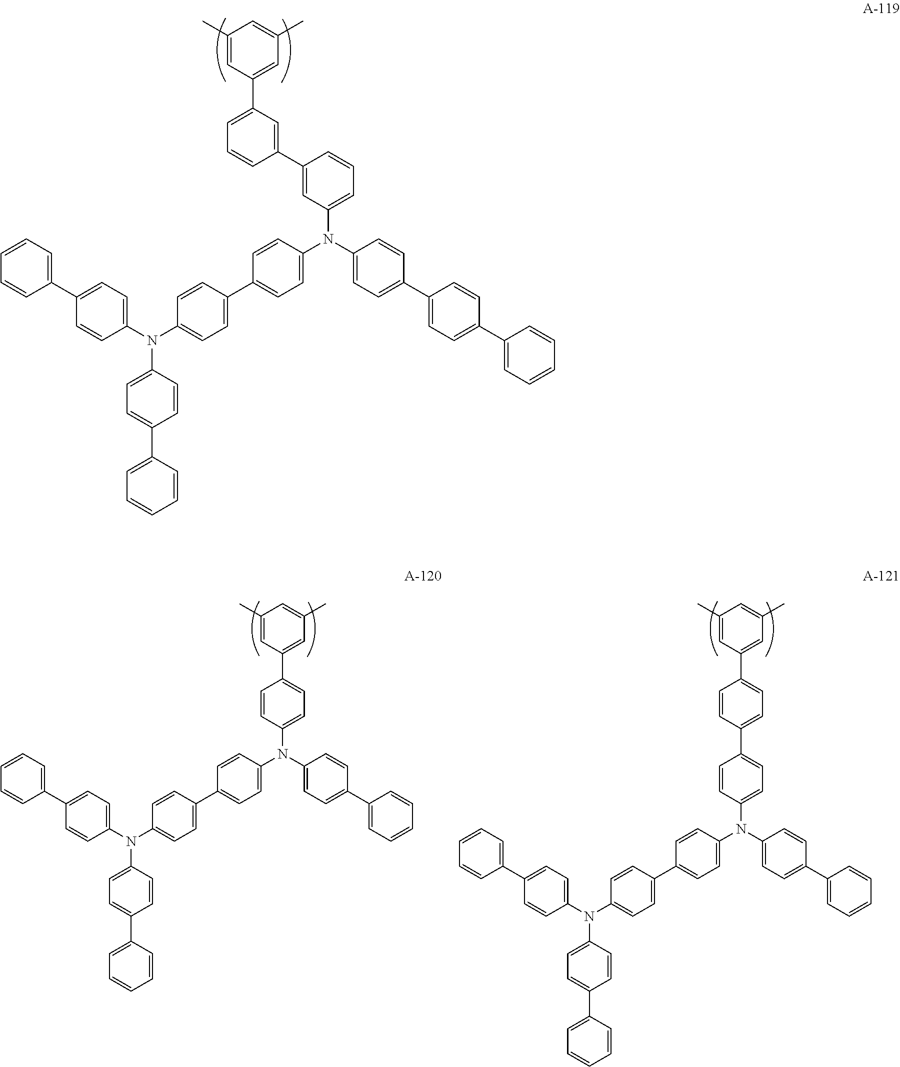

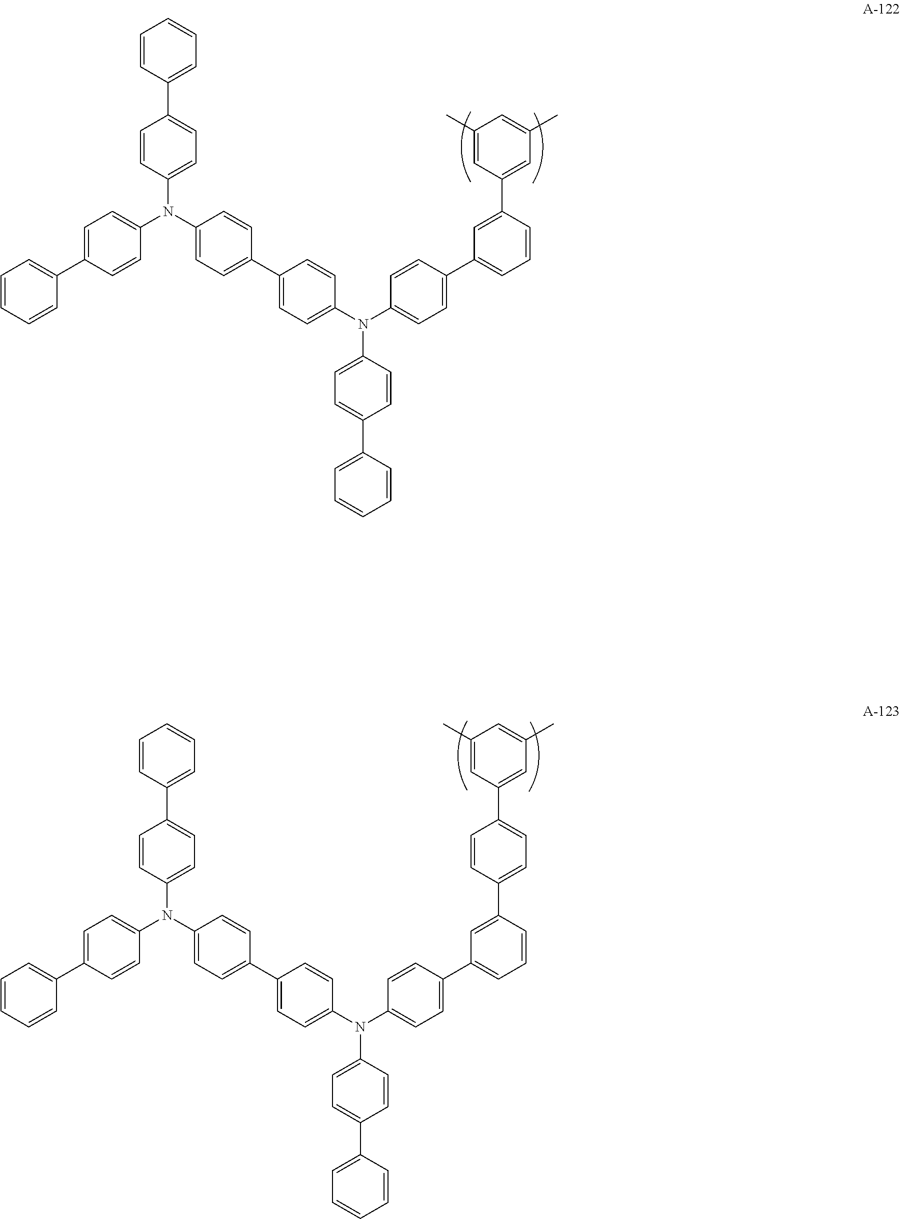

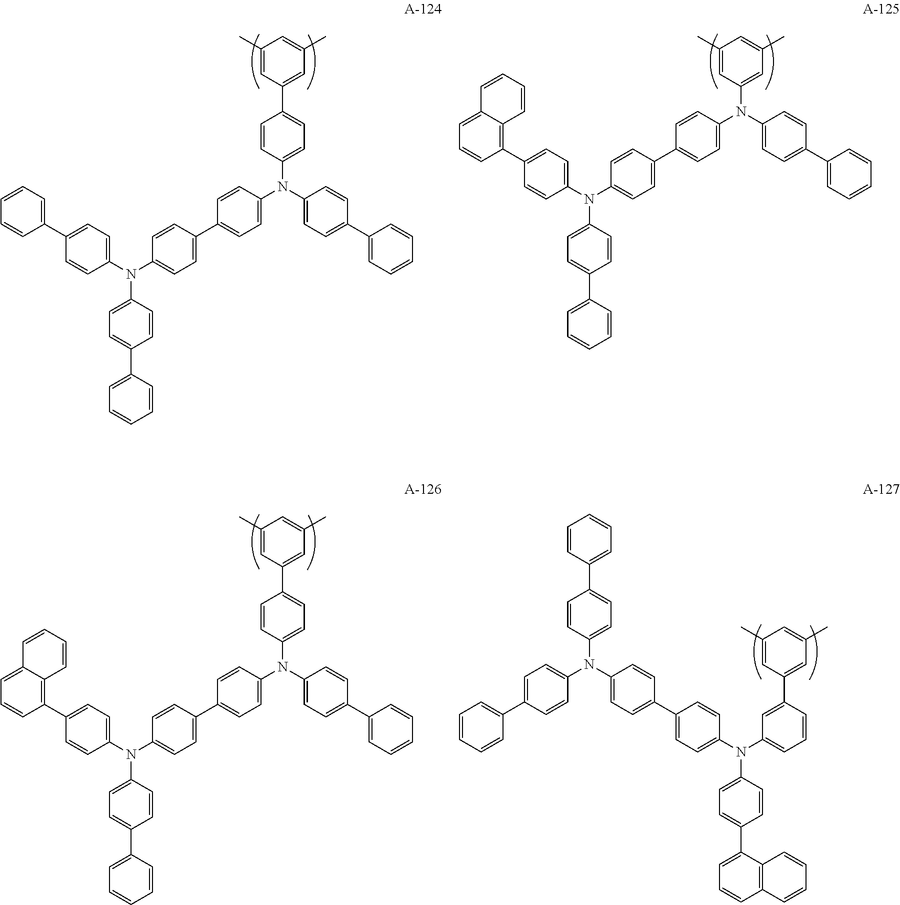

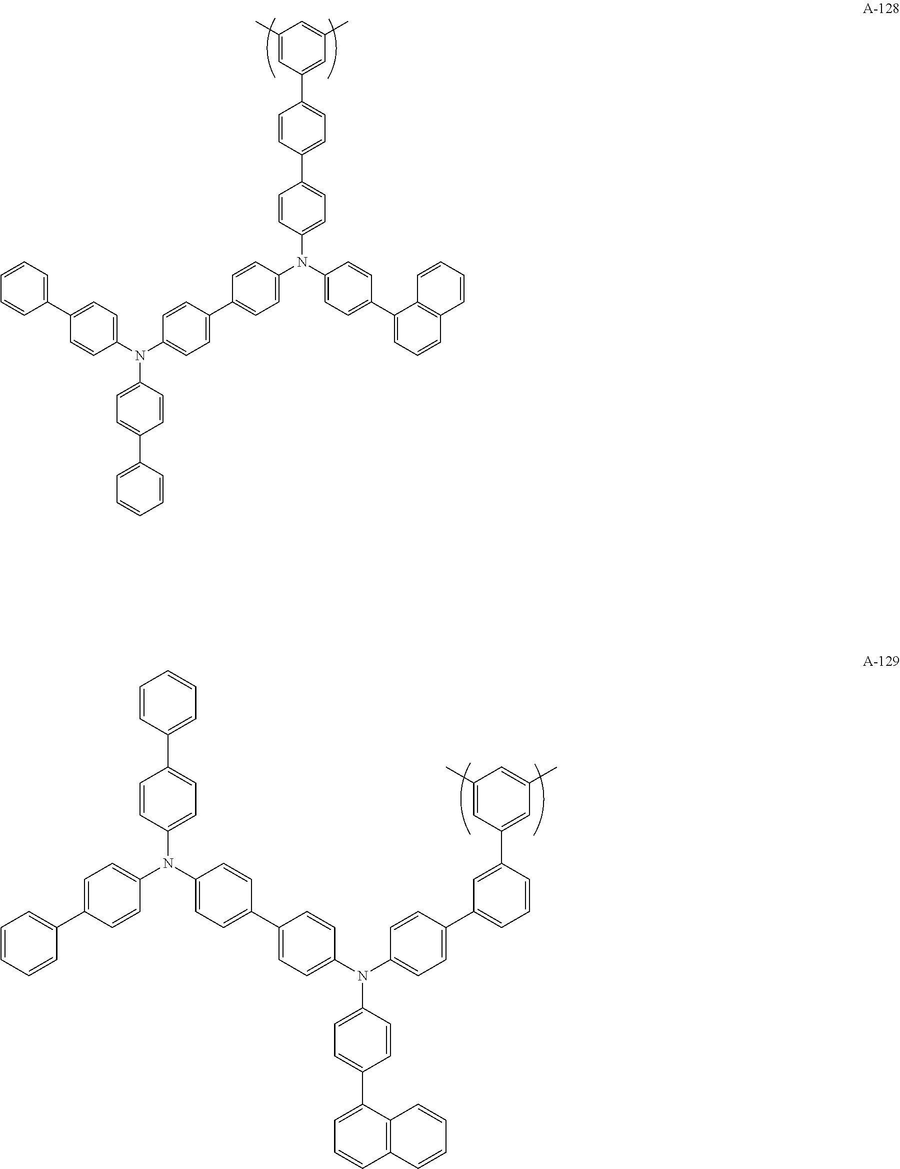

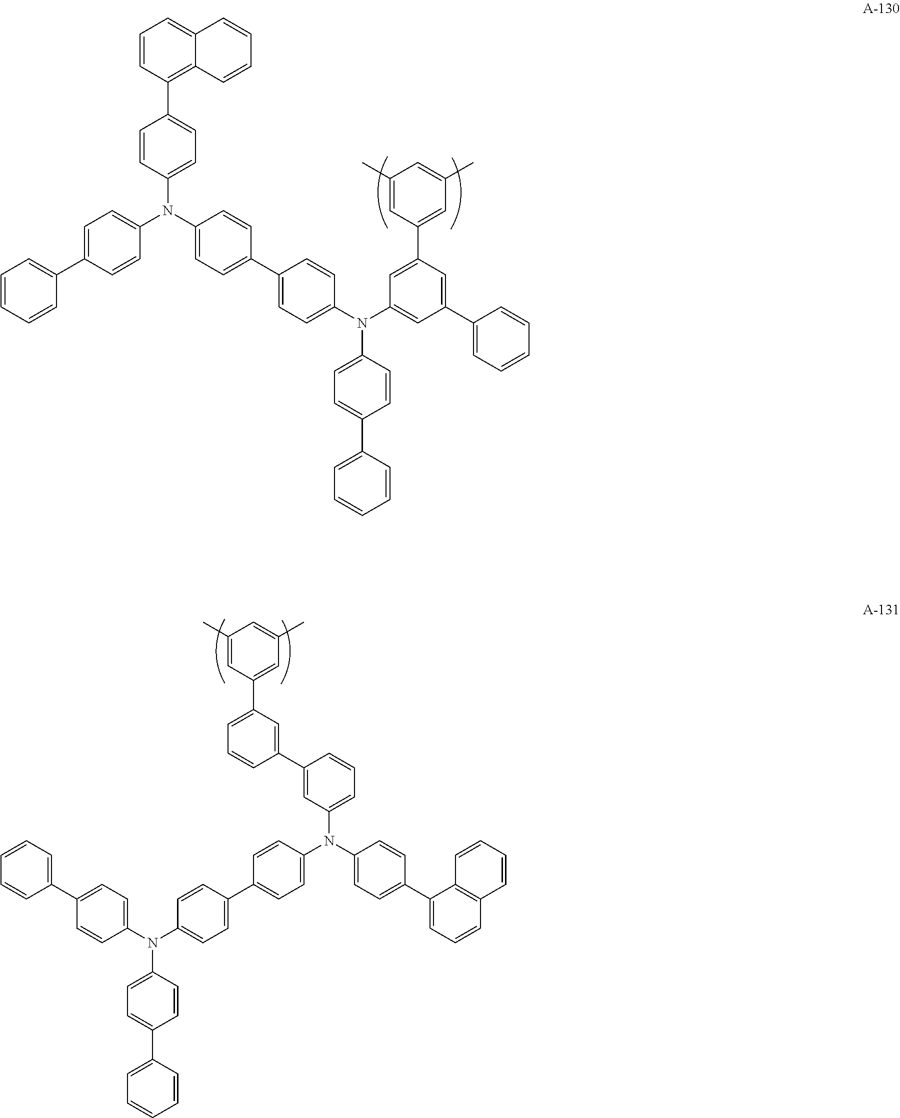

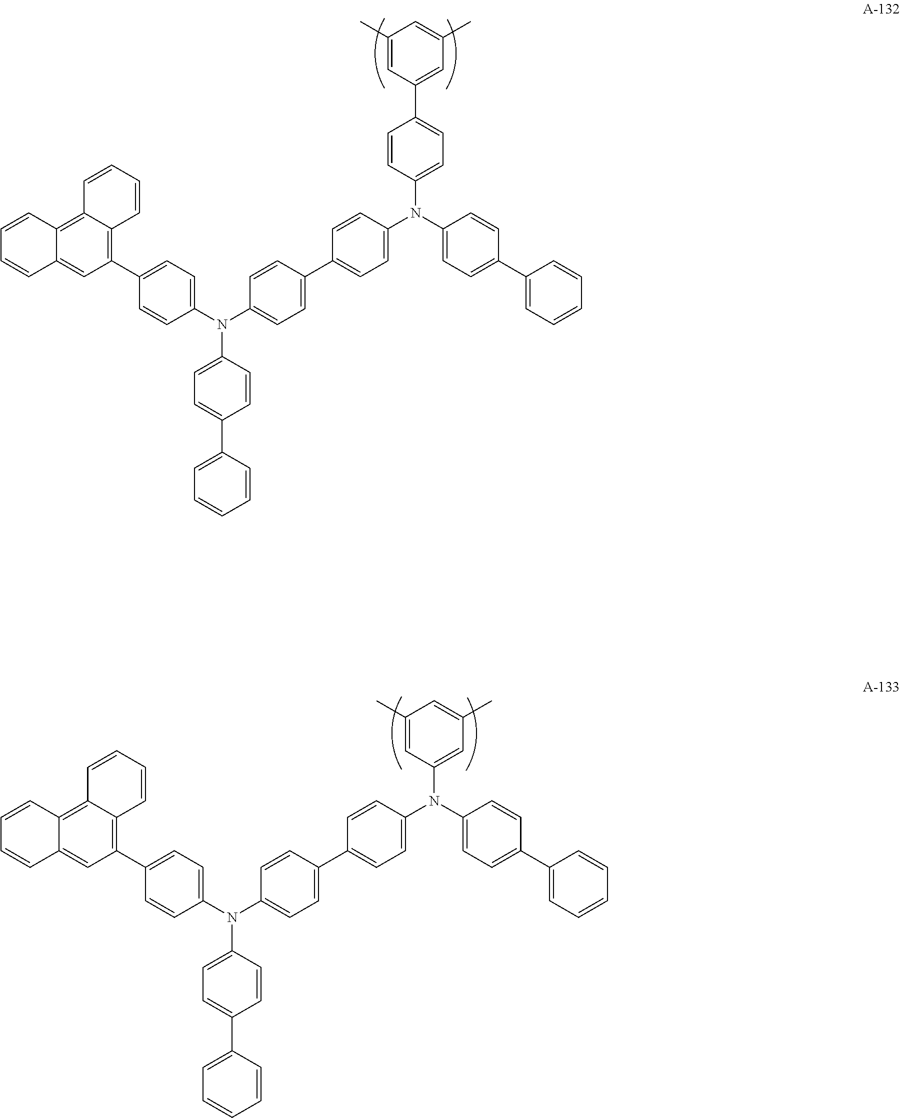

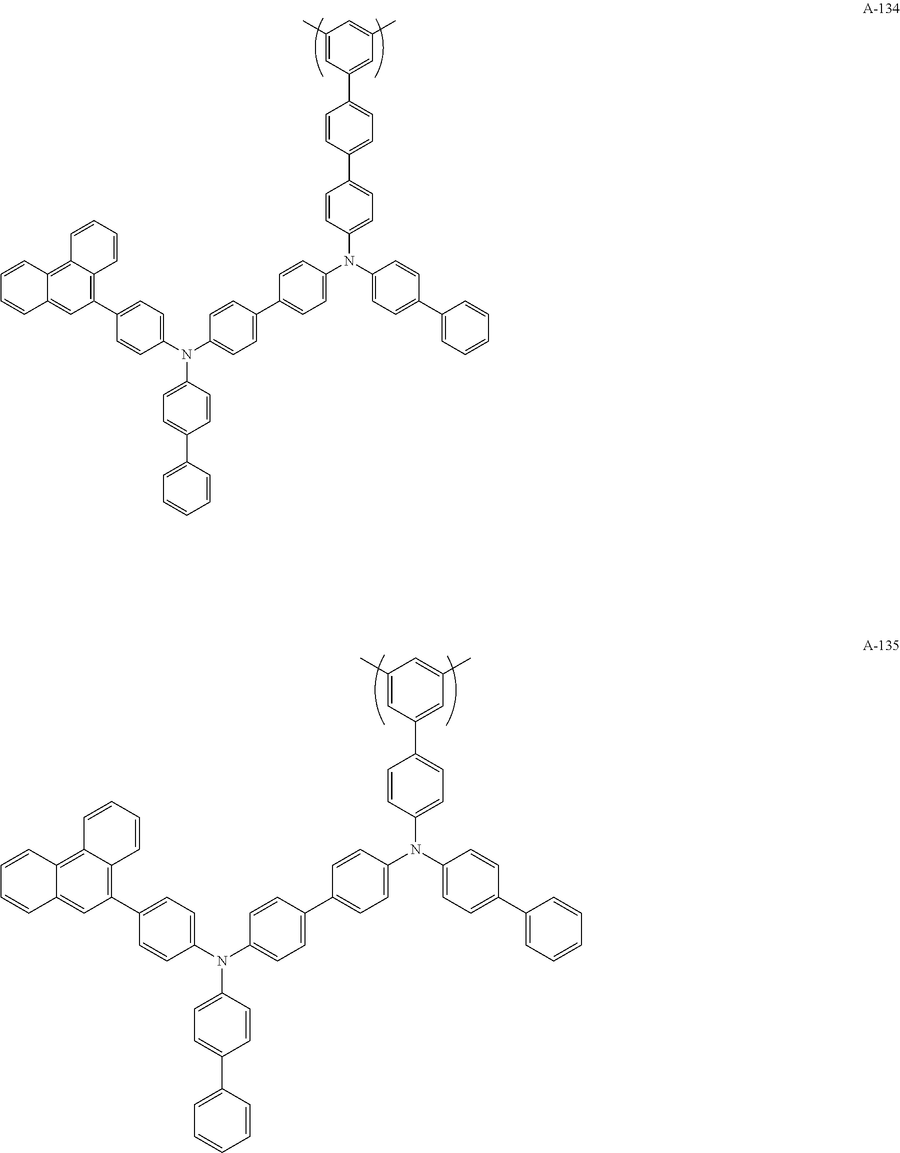

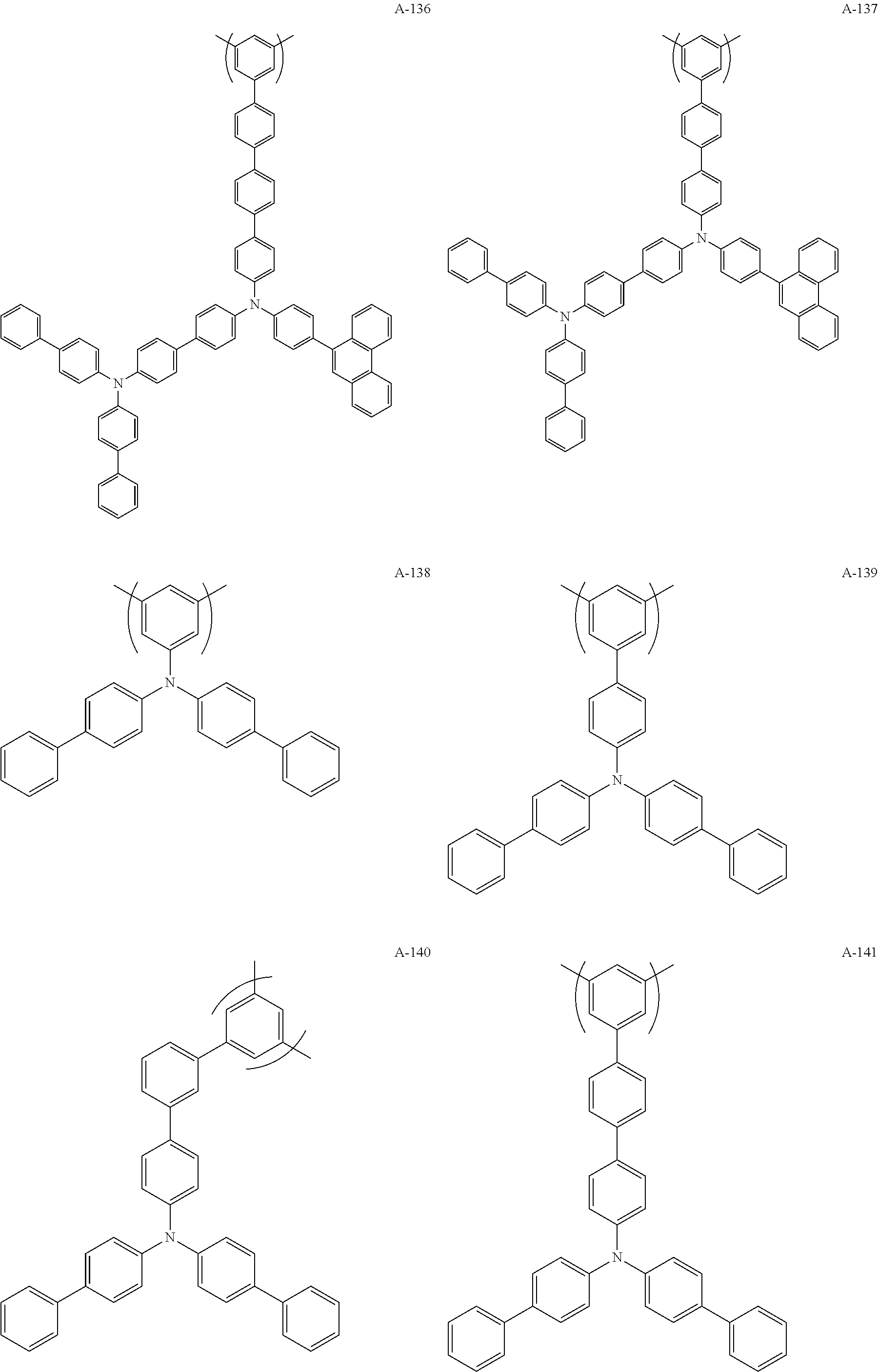

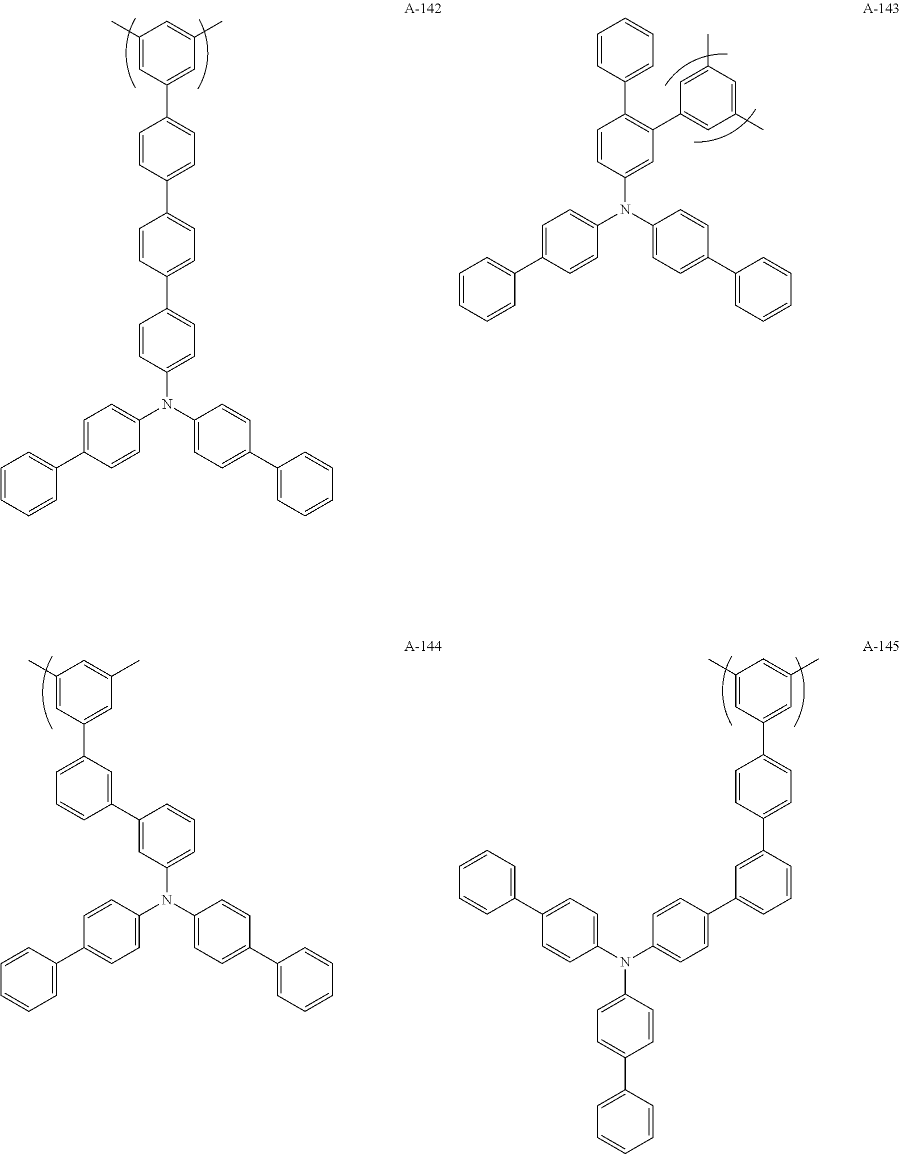

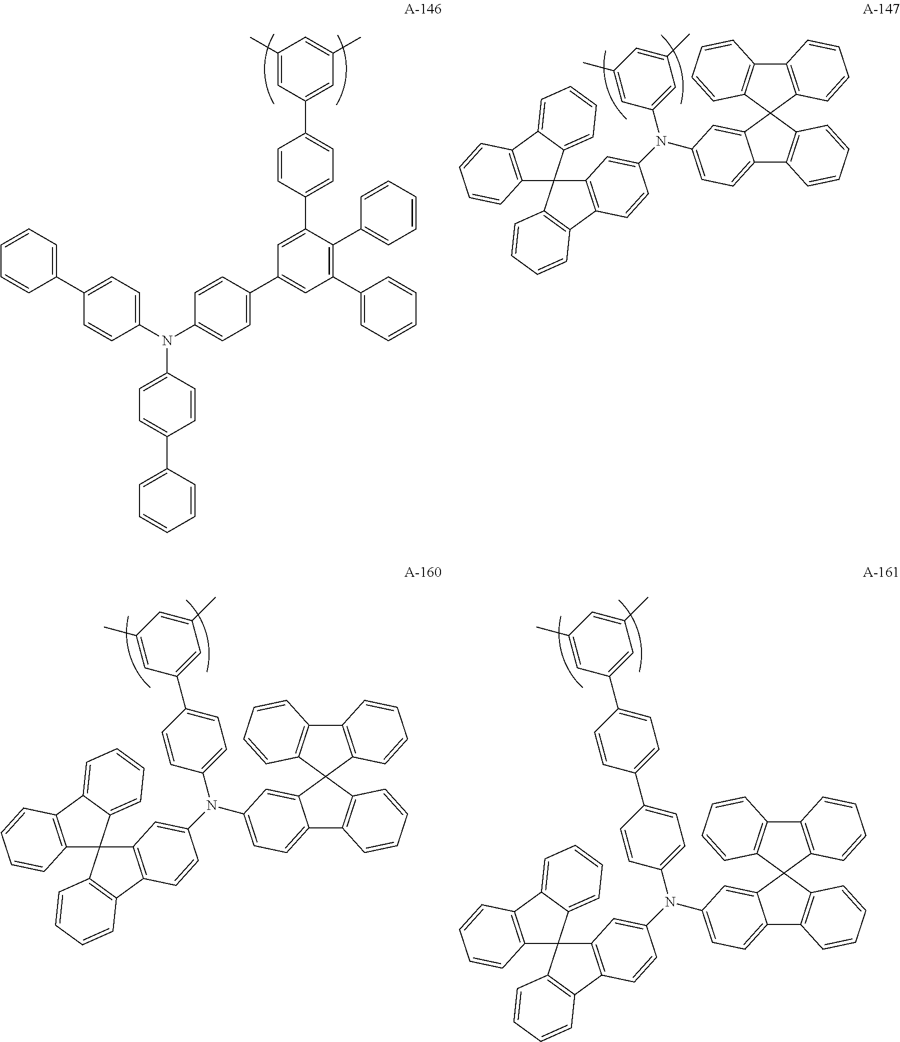

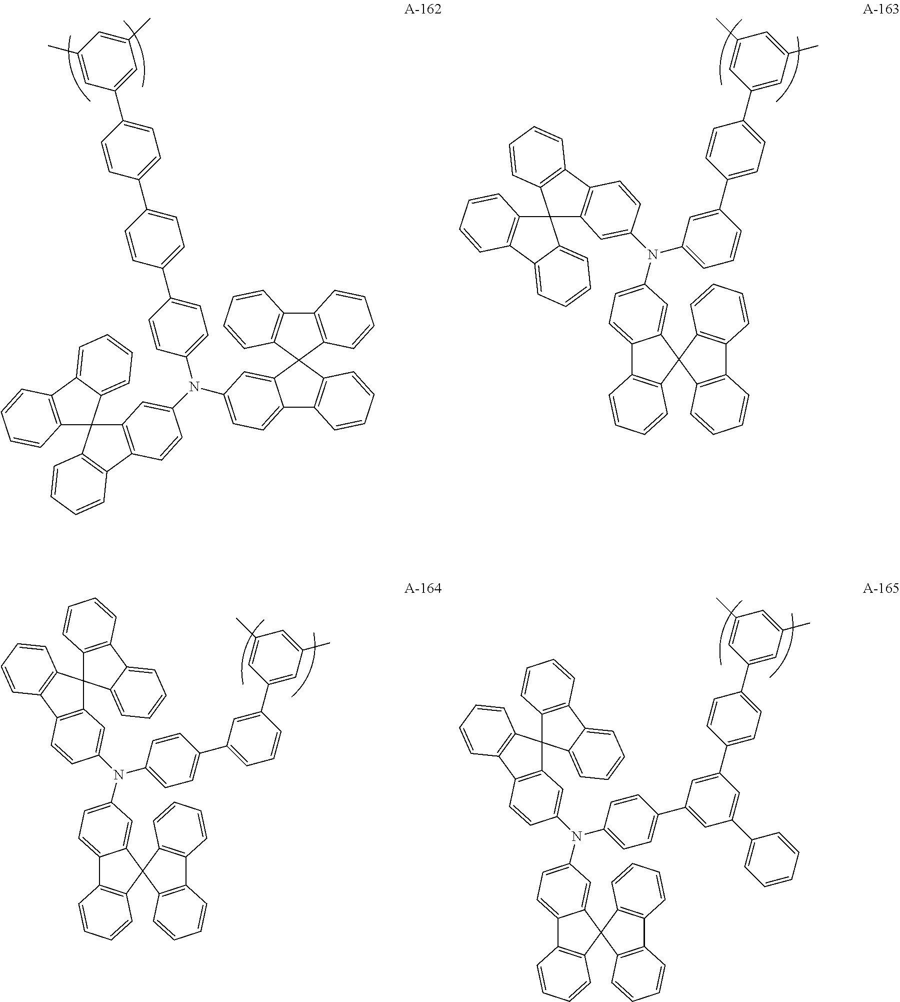

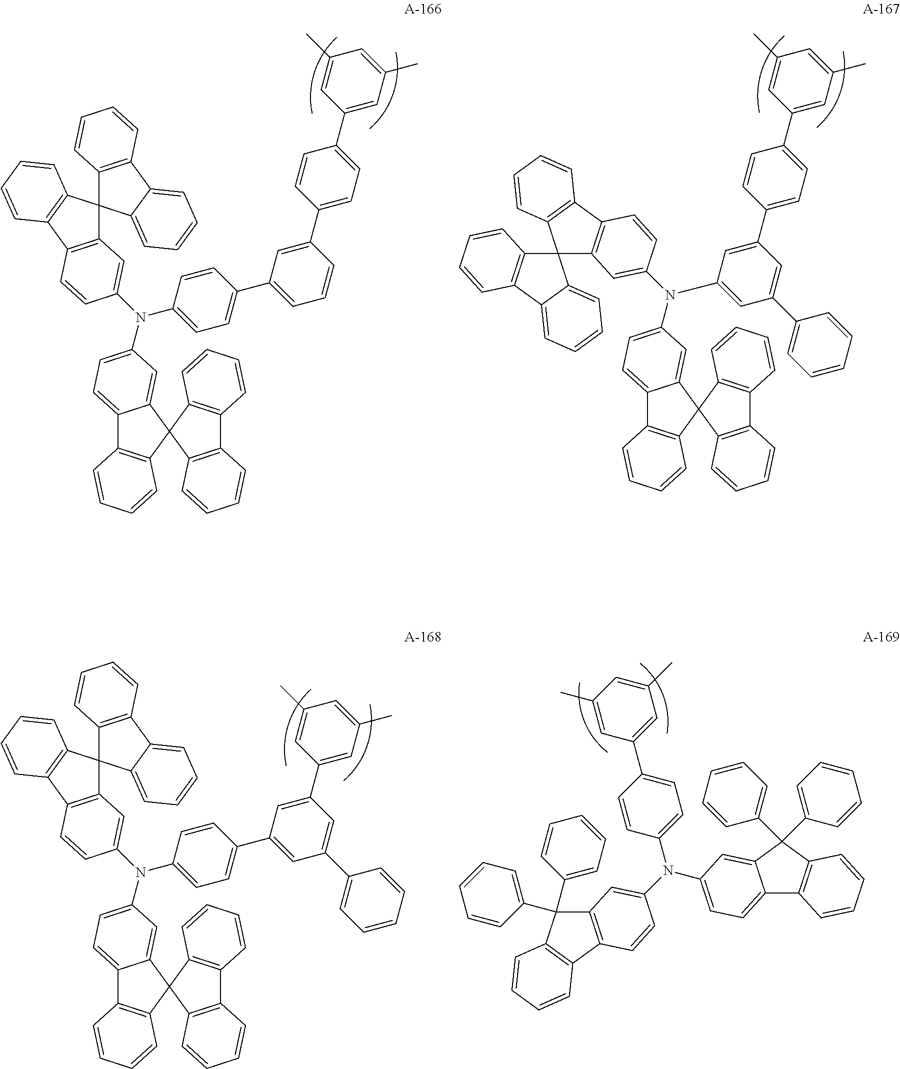

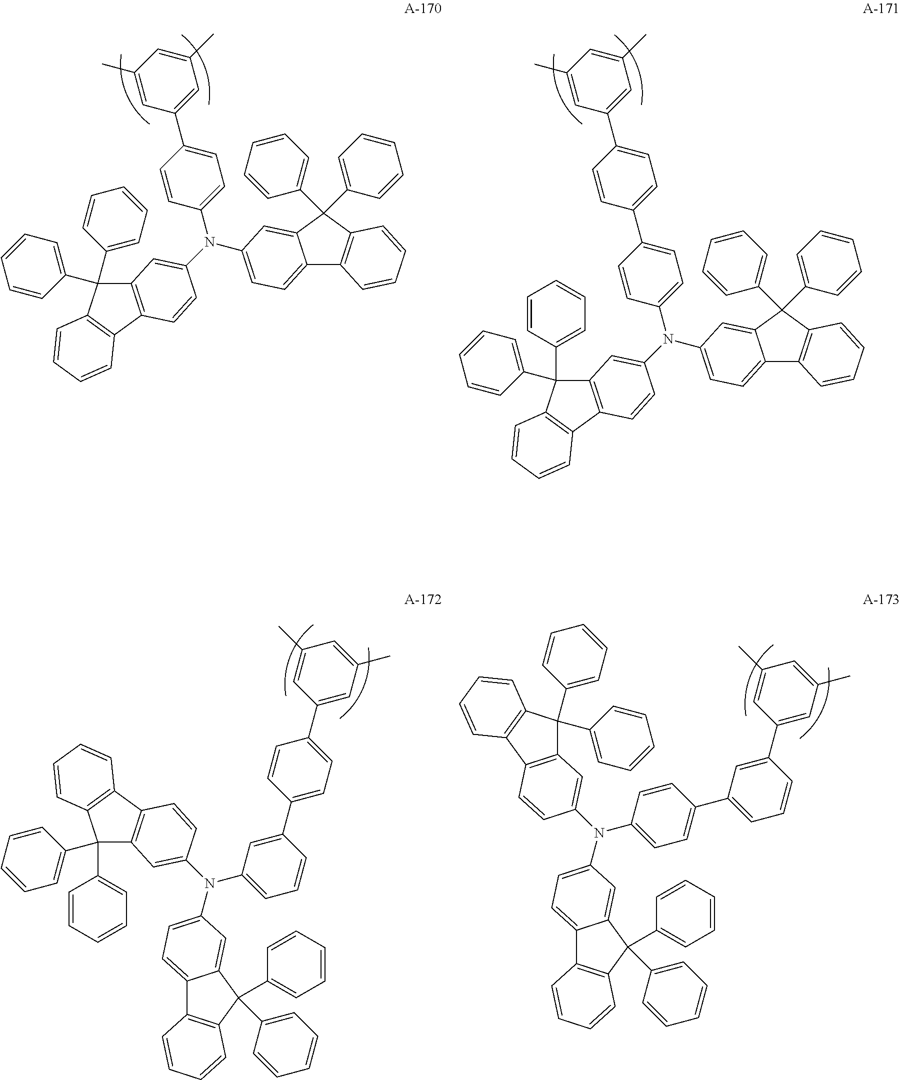

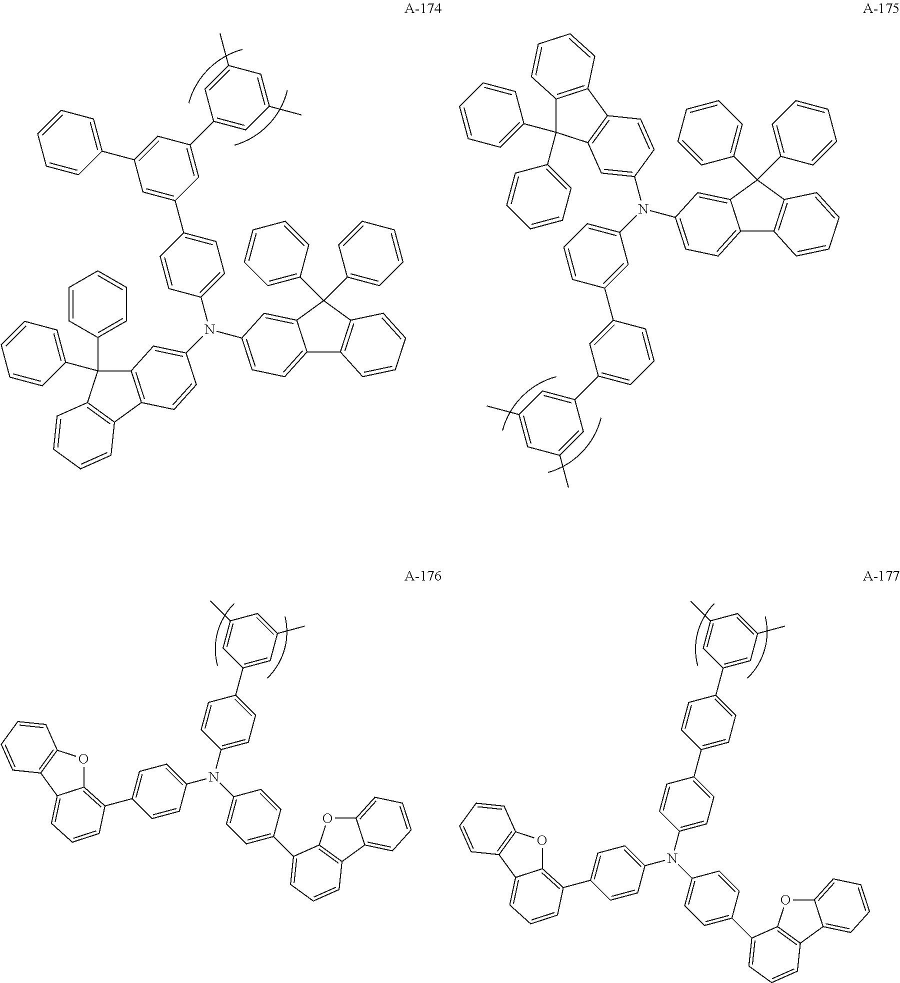

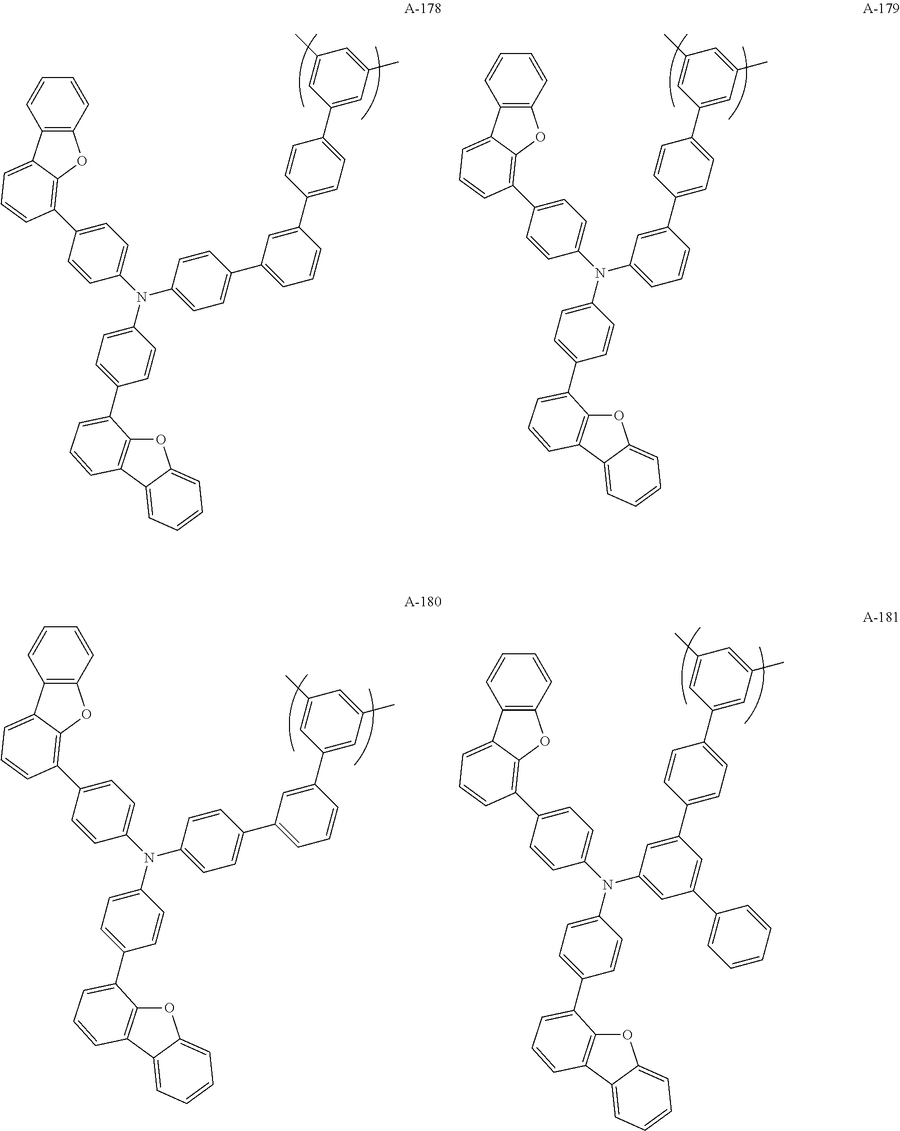

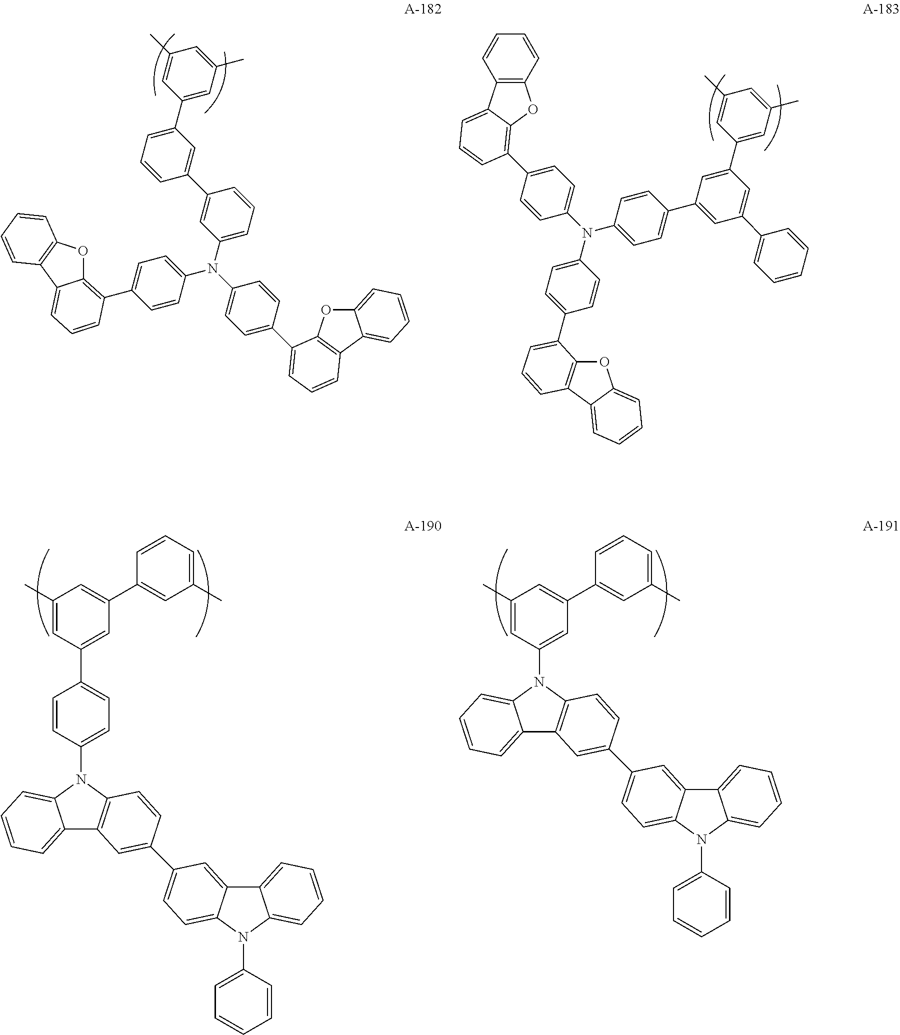

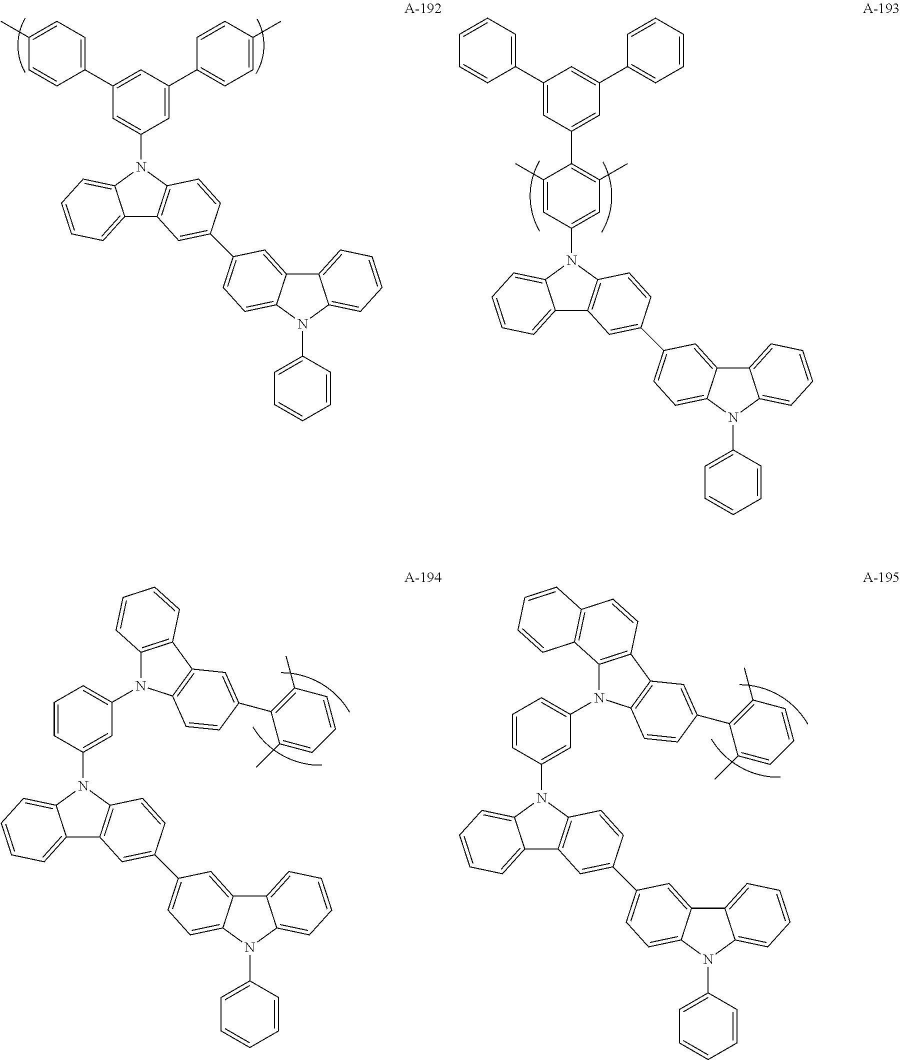

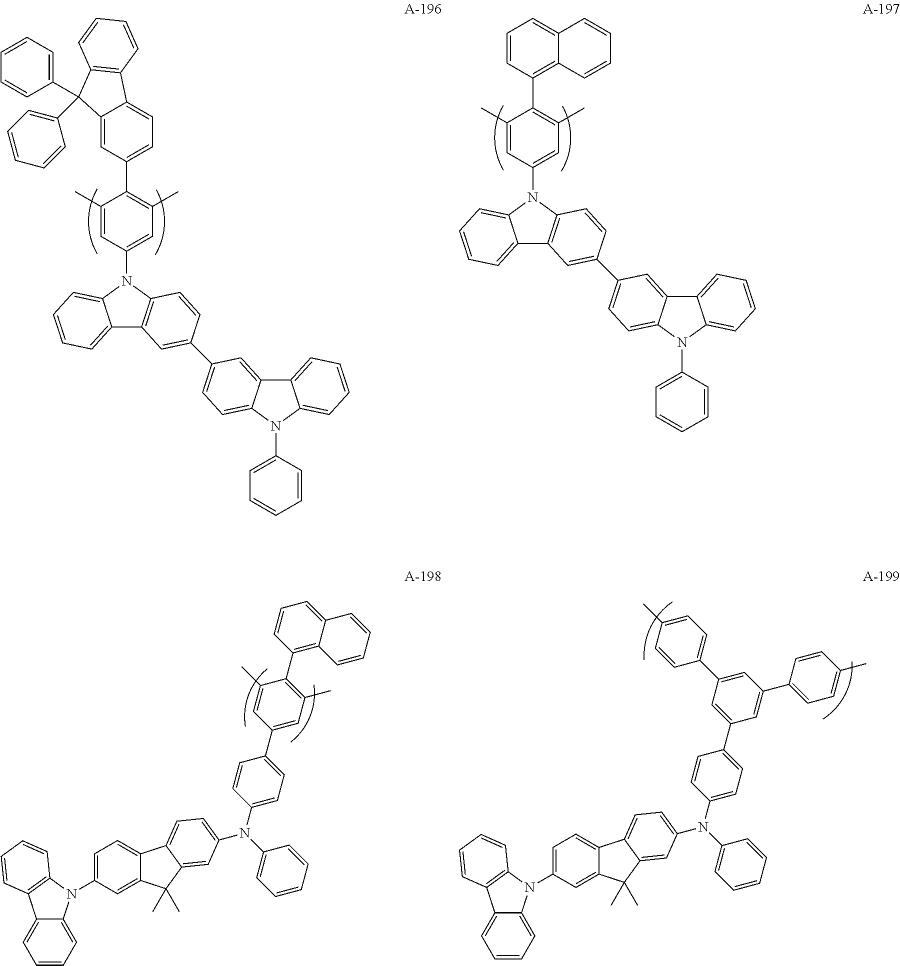

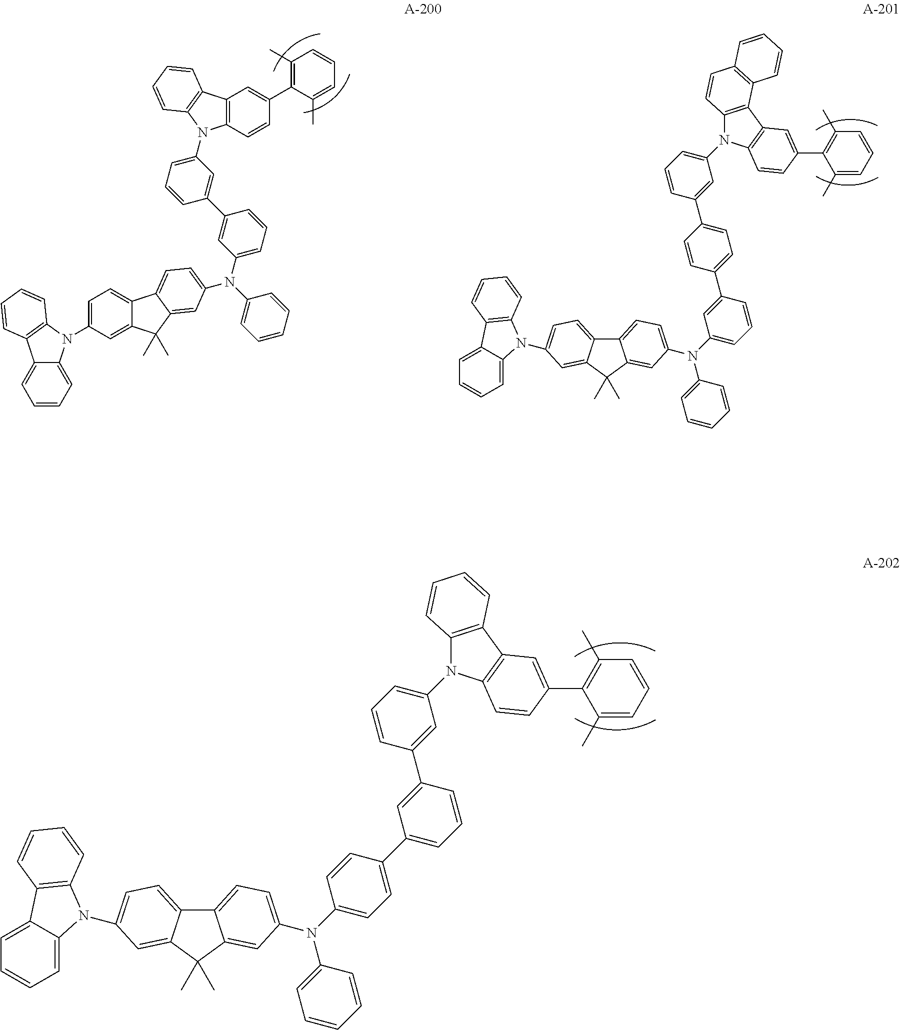

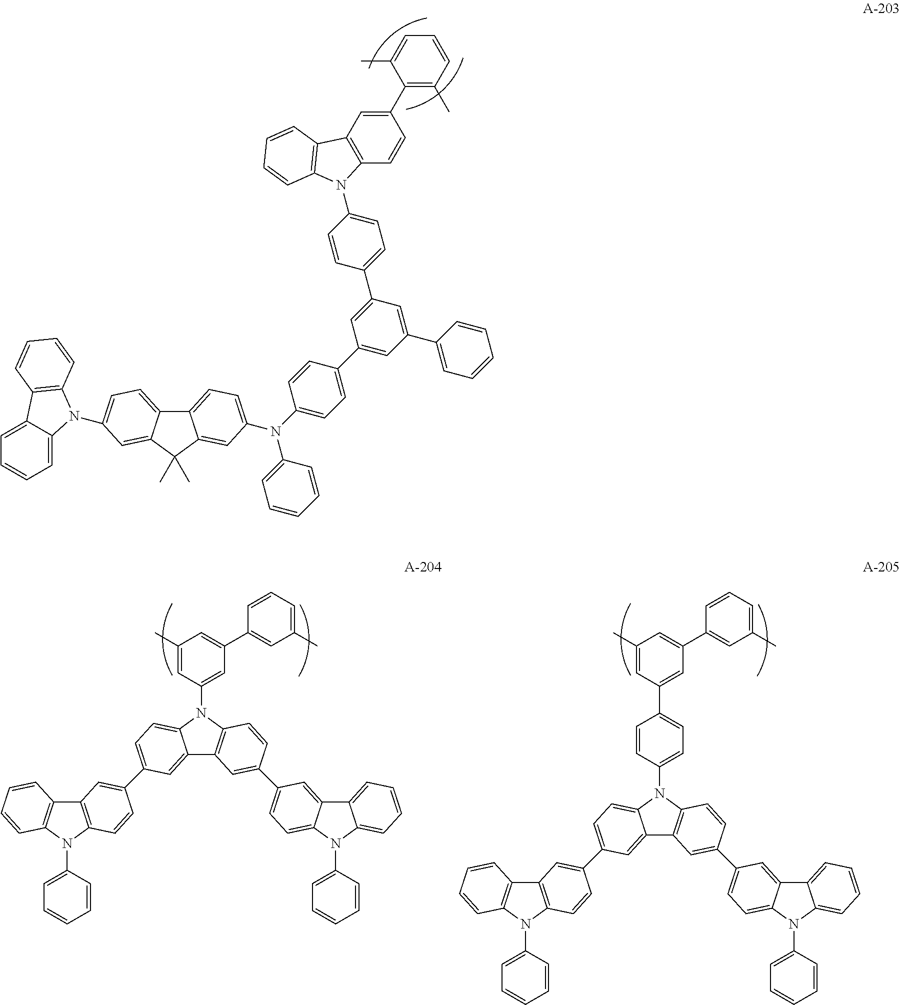

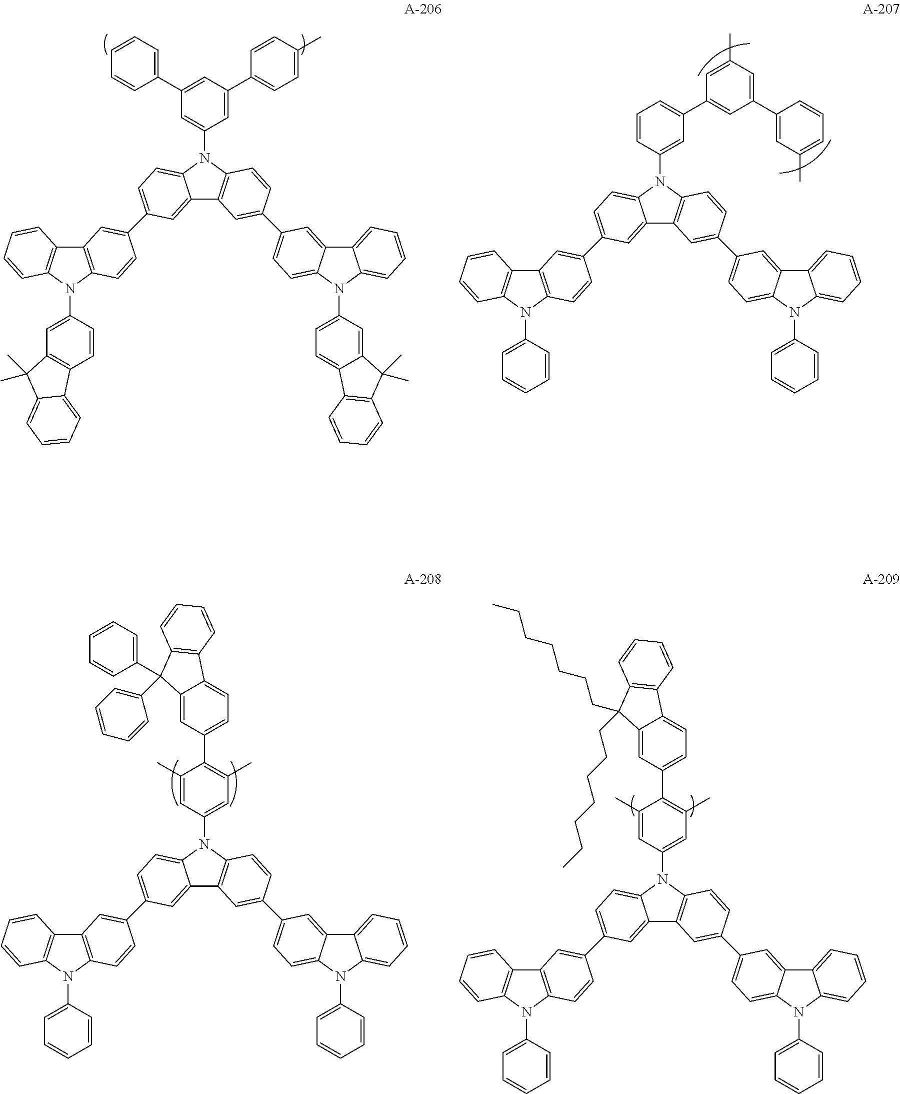

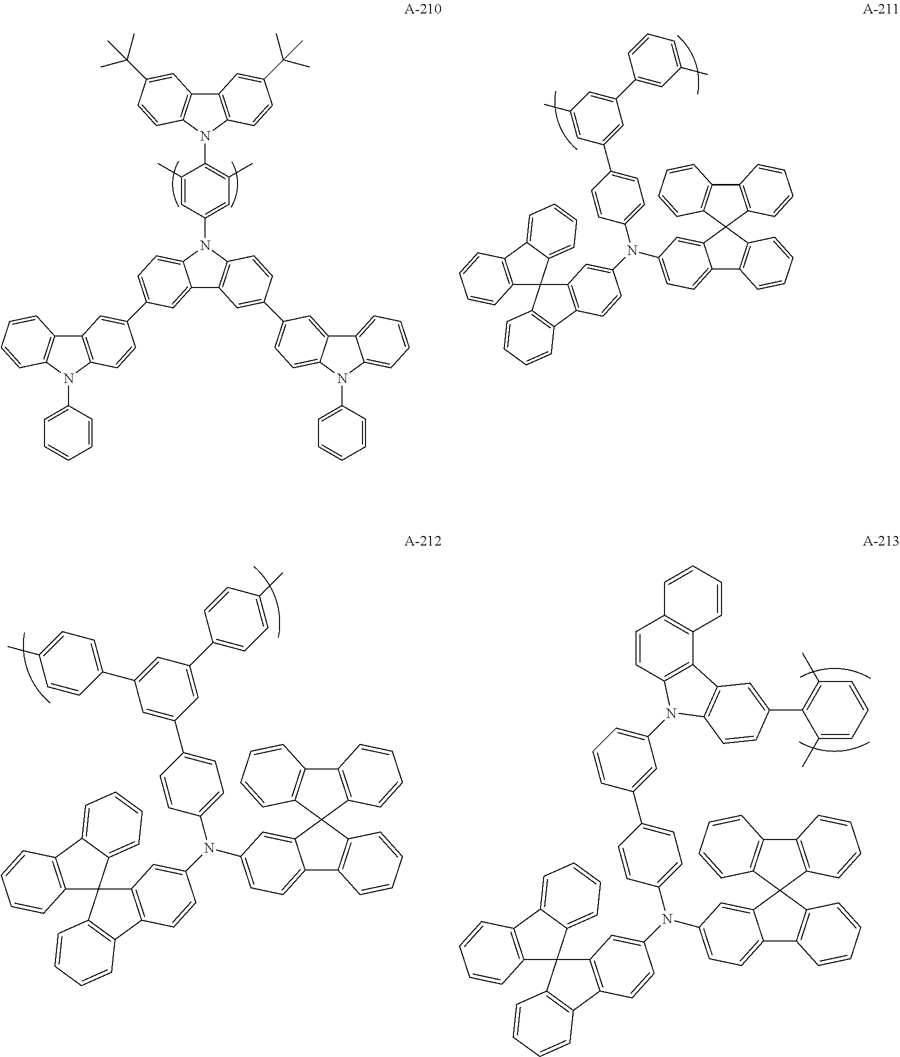

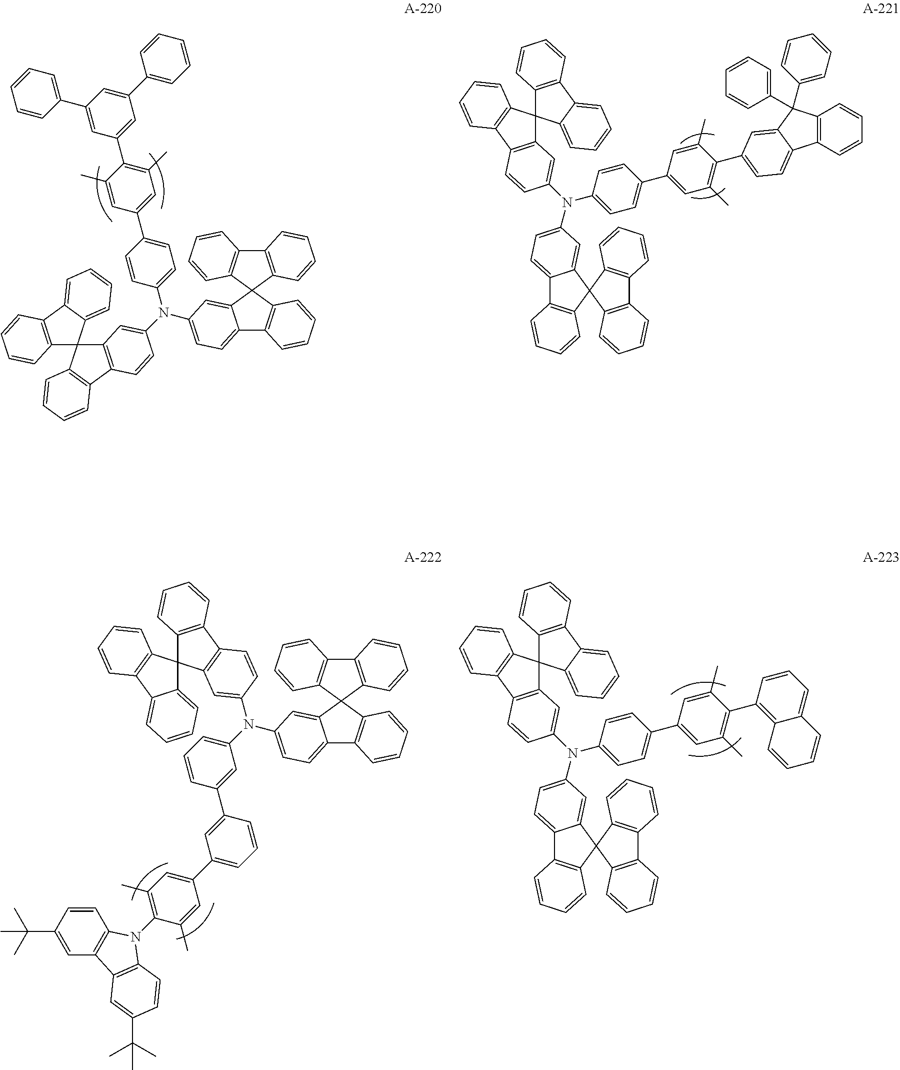

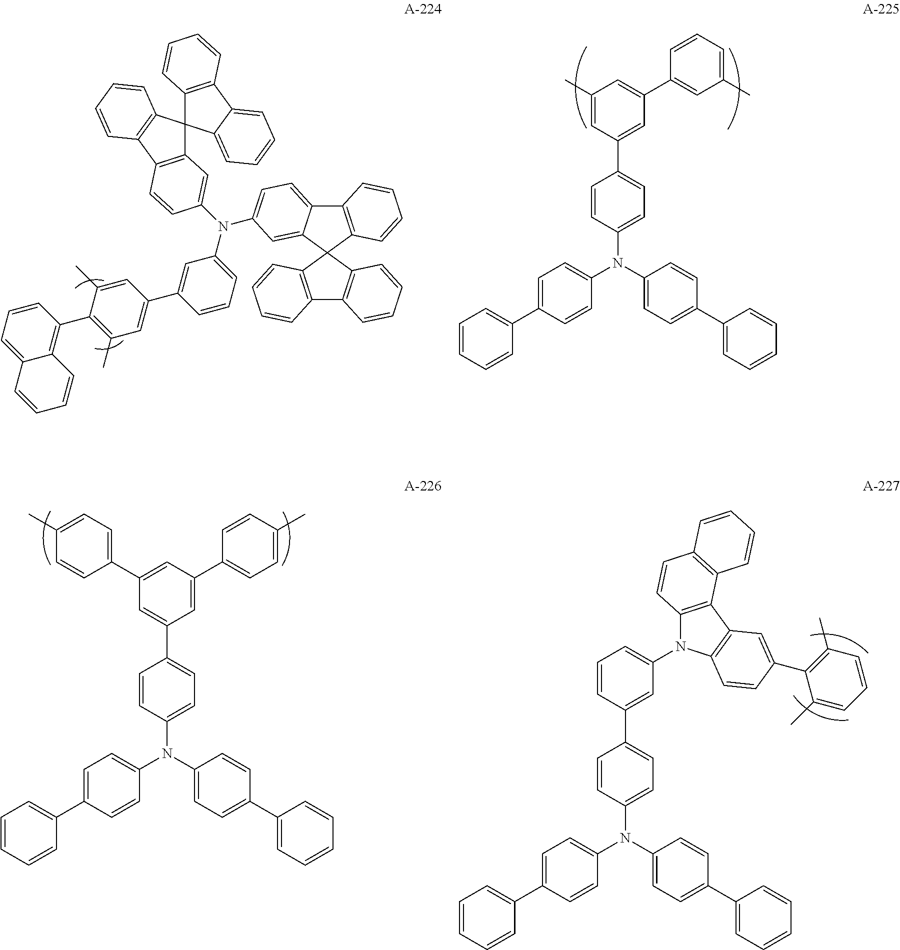

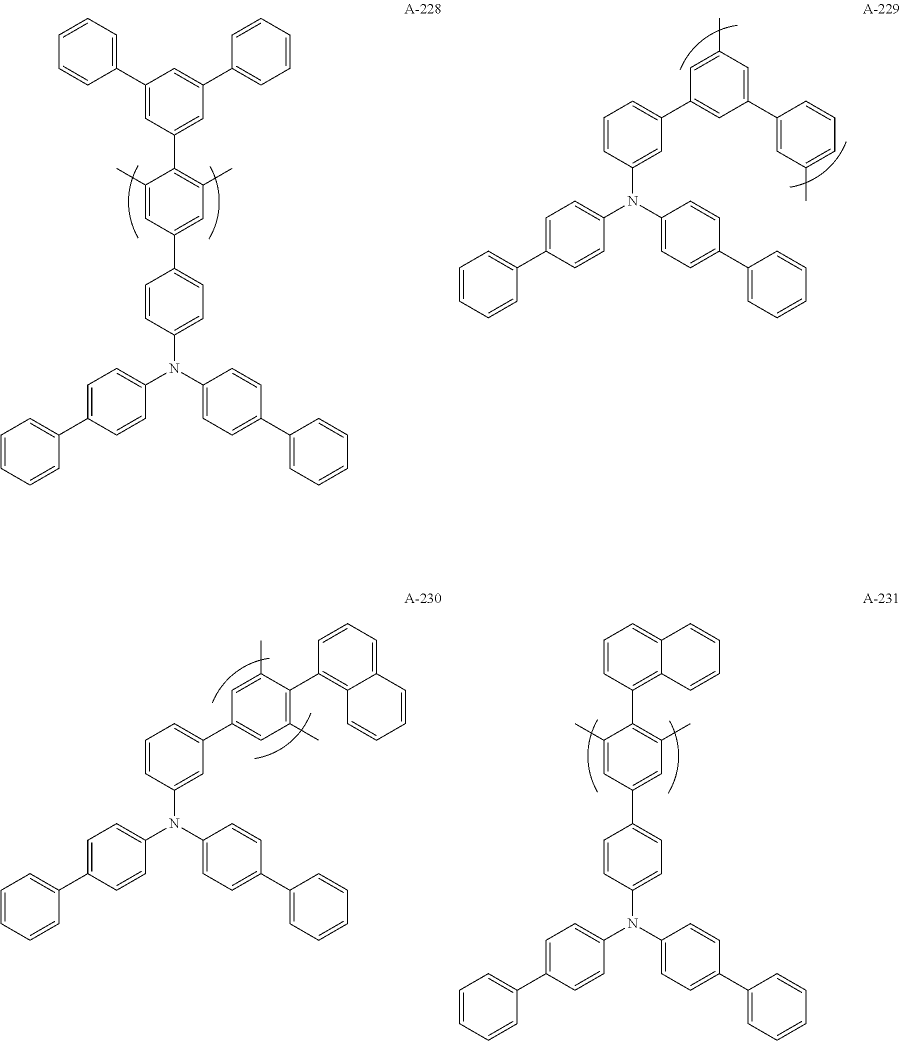

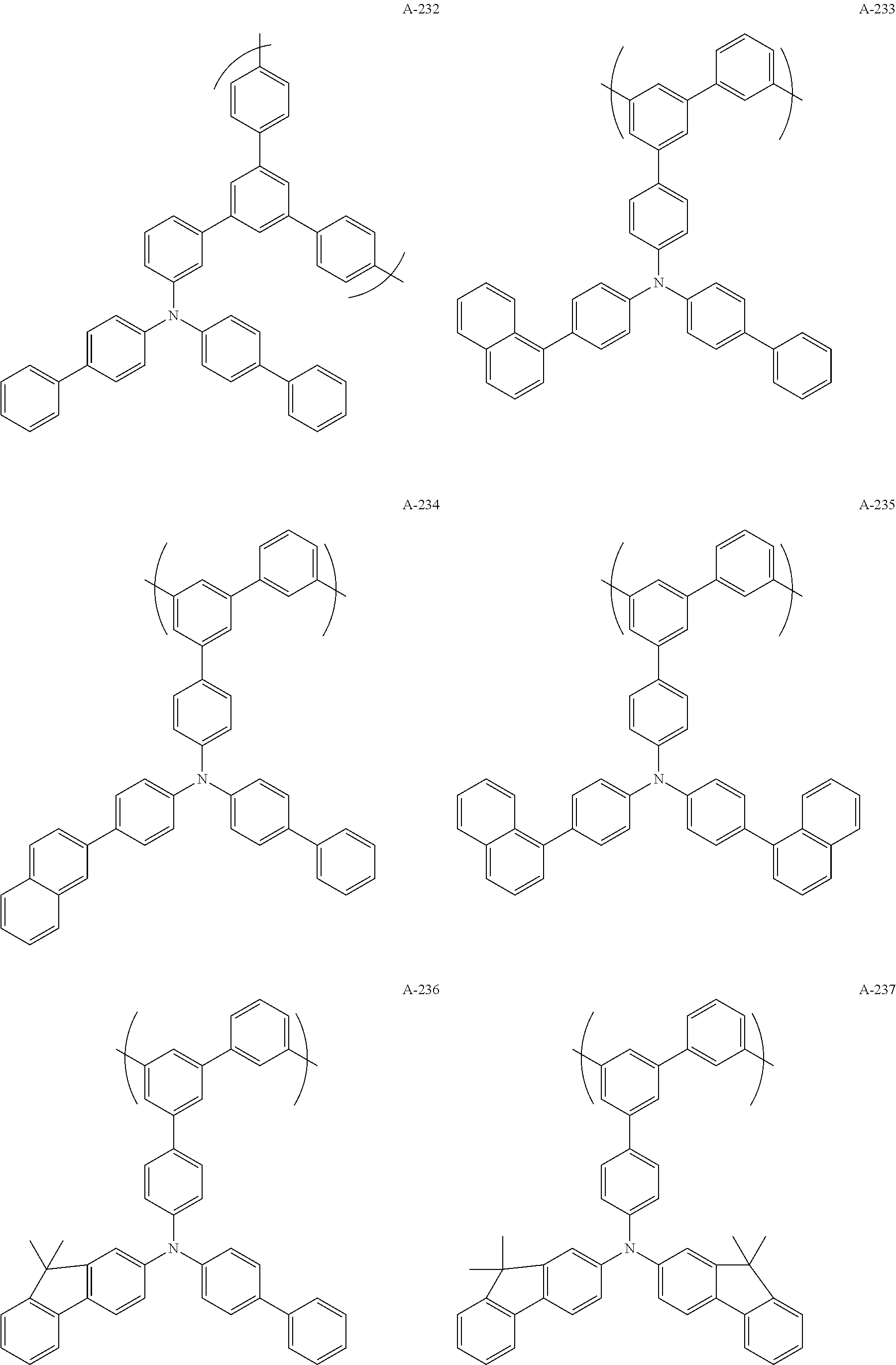

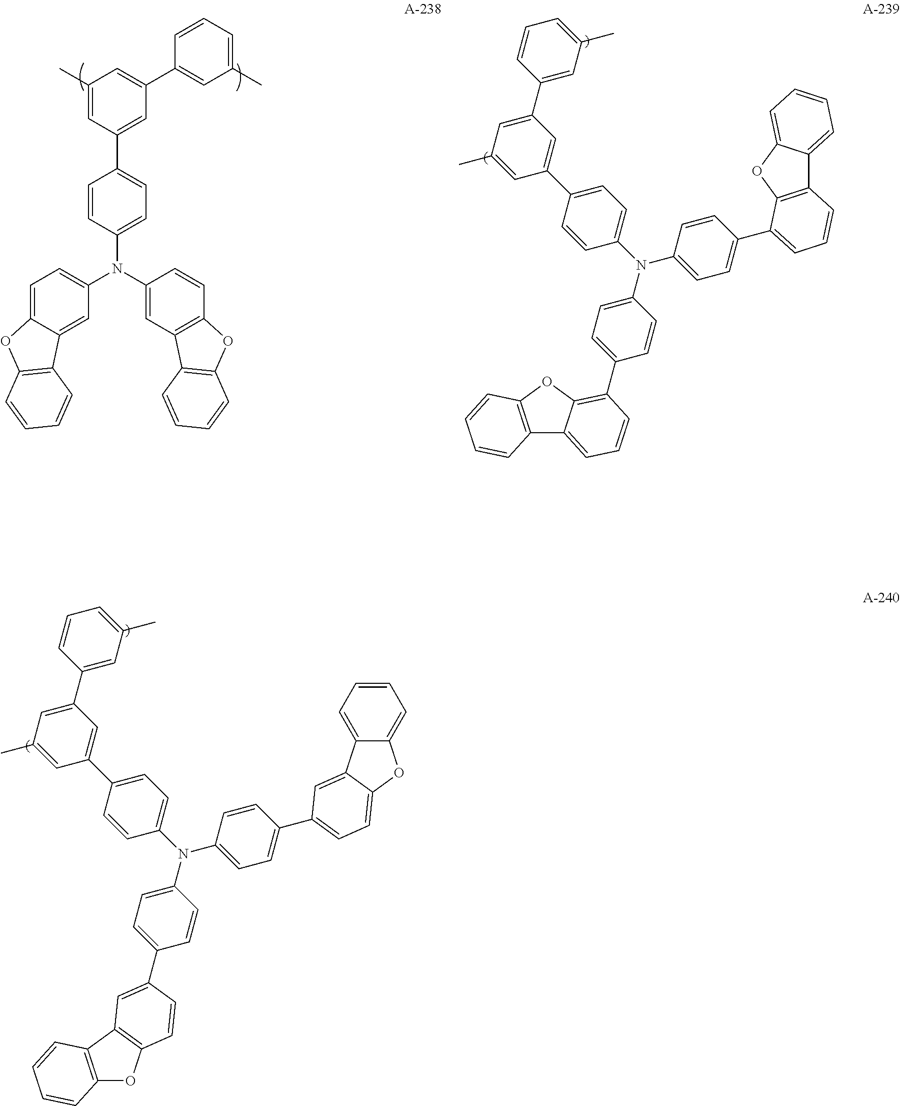

The structural unit (B) comprises at least one selected from an aromatic hydrocarbon ring group and an aromatic heterocyclic group.

The structural unit (B) is preferably represented by at least one selected from formulae (B1) to (B3) and more preferably represented by formula (B1):

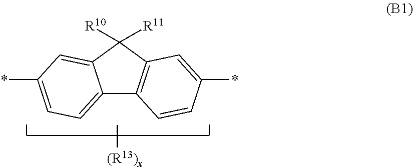

wherein:

each of R¹⁰ and R¹¹ is independently a hydrogen atom, an alkyl group having 1 to 60 carbon atoms, an aryl group, a mono valent aromatic heterocyclic group, an aralkyl group, or a substituted amino group;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

x is an integer of 0 to 3;

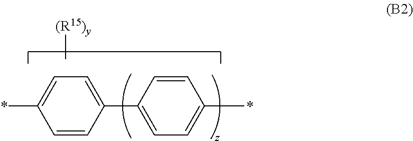

wherein:

each R¹⁵ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

y is an integer of 0 to 3; and

z is 0 or 1;

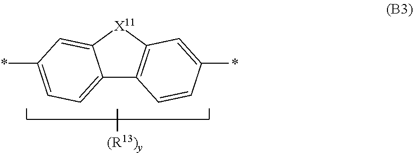

wherein:

X¹¹ is an oxygen atom or a sulfur atom;

each R¹³ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton; and

y is an integer of 0 to 3.

Each variable of formulae (B1) to (B3) is described below.

The subscript x is preferably an integer of 0 to 2 and more preferably 1 or 2.

The subscript y is preferably an integer of 1 to 3, more preferably 2 or 3, and further more preferably 2.

Each of R¹⁰ and R¹¹ is preferably an alkyl group having 1 to 60 carbon atoms, an aryl group, a mono valent aromatic heterocyclic group, an aralkyl group, or a substituted amino group. In view of increasing the solubility of the cross-conjugated polymer to a solvent, preferred is a linear or branched alkyl group having preferably 1 to 60, more preferably 4 to 50, further more preferably 4 to 30, and still further more preferably 4 to 10 carbon atoms.

Examples of the alkyl group having 1 to 60 carbon atoms for R¹⁰ and R¹¹ include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups). Preferred are a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups). More preferred are a n-butyl group, a sec-butyl group, a t-butyl group, a n-pentyl group, a sec-pentyl group, a n-hexyl group, a n-heptyl group, a n-an octyl group, and an isooctyl group.

Each of R¹³ and R¹⁵ is preferably a tertiary alkyl group having 4 to 60 carbon atoms, an alkoxy group having 1 to 20 carbon atoms, or a cycloalkoxy group having 3 to 10 ring carbon atoms and more preferably an alkoxy group having 1 to 20 carbon atoms. The alkoxy group has preferably 4 to 20, more preferably 4 to 15, and further more preferably 4 to 10 carbon atoms.

Examples of the alkoxy group include a n-butoxy group, a sec-butoxy group, a t-butoxy group, a n-pentyloxy group, a sec-pentyloxy group, a n-hexyloxy group, a n-heptyloxy group, a n-octyloxy group, and an isooctyloxy group.

The aryl group, the mono valent aromatic heterocyclic group, the aryloxy group having 6 to 30 ring carbon atoms, the aralkyl group, the substituted amino group, the substituted silyl group, the cyano group, and the halogen atom for R¹⁰, R¹¹, R¹³, and R¹⁵ are the same as those mentioned above with respect to R¹.

Examples of the structural unit (B) are shown below, although not limited thereto.

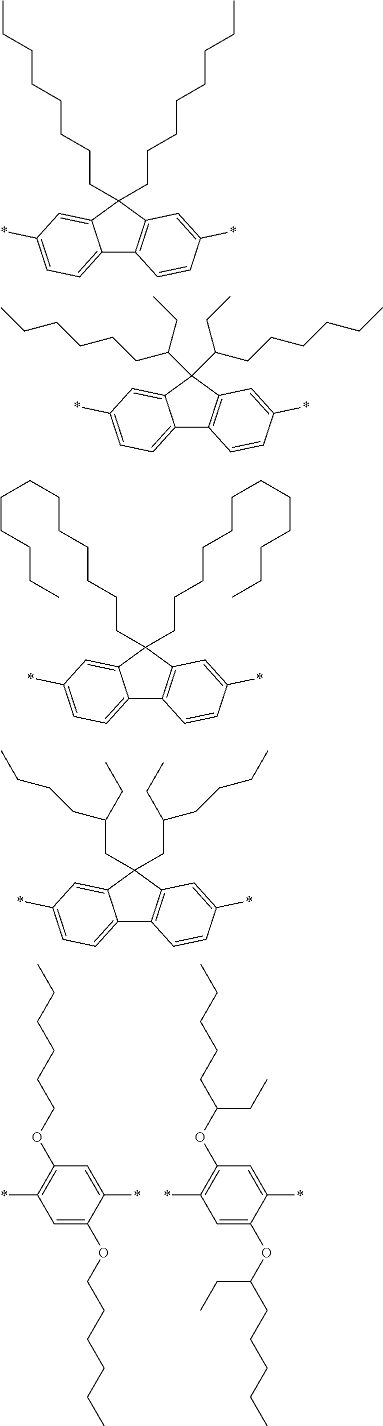

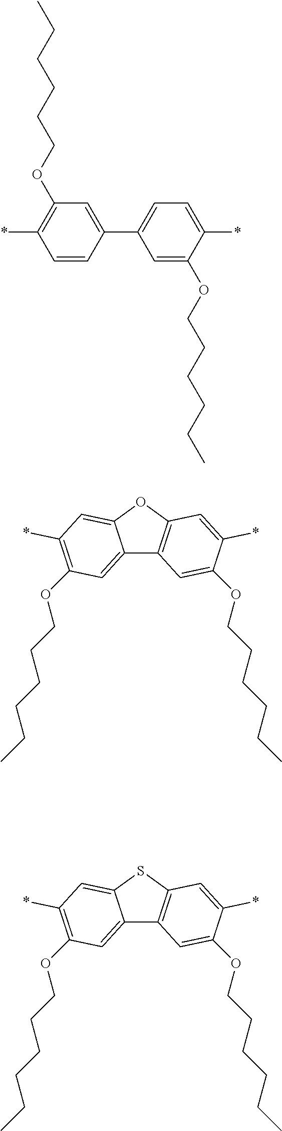

Of the above structural units B, more preferred are:

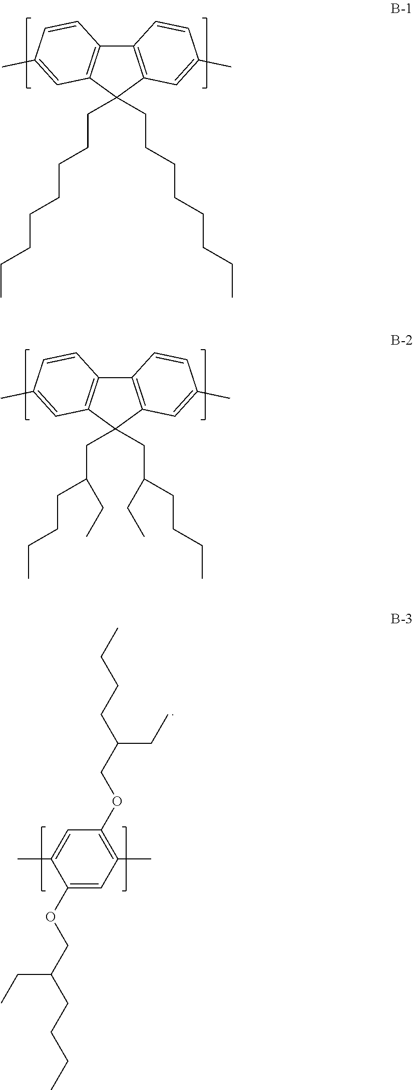

The cross-conjugated polymer of the invention preferably comprises a cross-linkable structural unit (C) as a repeating unit. The structural unit (C) is preferably represented by formula (C1) or (C2).

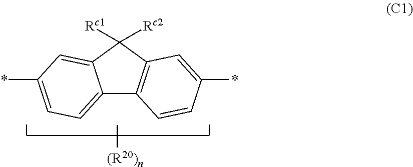

wherein:

each of R^{c1} and R^{c2} is independently a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

n is an integer of 0 to 3;

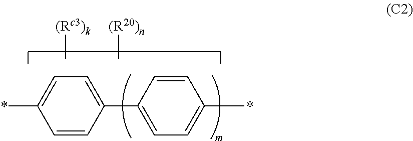

wherein:

each R^{c3} is a group comprising a small ring having 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;

each R²⁰ is independently a tertiary alkyl group having 4 to 60 carbon atoms, an aryl group, a mono valent aromatic heterocyclic group, an alkoxy group having 1 to 20 carbon atoms, a cycloalkoxy group having 3 to 10 ring carbon atoms, an aryloxy group having 6 to 30 ring carbon atoms, an aralkyl group, a substituted amino group, a substituted silyl group, a cyano group, or a halogen atom;

* is a bonding site in a main skeleton;

m is 0 or 1;

n is an integer of 0 to 3; and

k is an integer of 1 to 3.

Each of R^{c1} and R^{c2} is preferably a group comprising a small ring having 3 to 4 ring atoms.

Examples of the small ring having 3 to 4 ring atoms for R^{c1} and R^{c2} include a cyclopropyl group, a cyclobutyl group, an epoxy group, an oxetanyl group, a diketenyl group, and an epithio group.

Example of the group comprising a small ring having 3 to 4 ring atoms includes a group comprising a fused ring system wherein an unsubstituted aromatic hydrocarbon ring having 6 to 60 ring carbon atoms and a cyclobutane ring are fused to each other, such as a residue derived from a benzocyclobutene (BCB) by removing at least one hydrogen atom.

Examples of R^{c1} and R^{c2} are shown below.

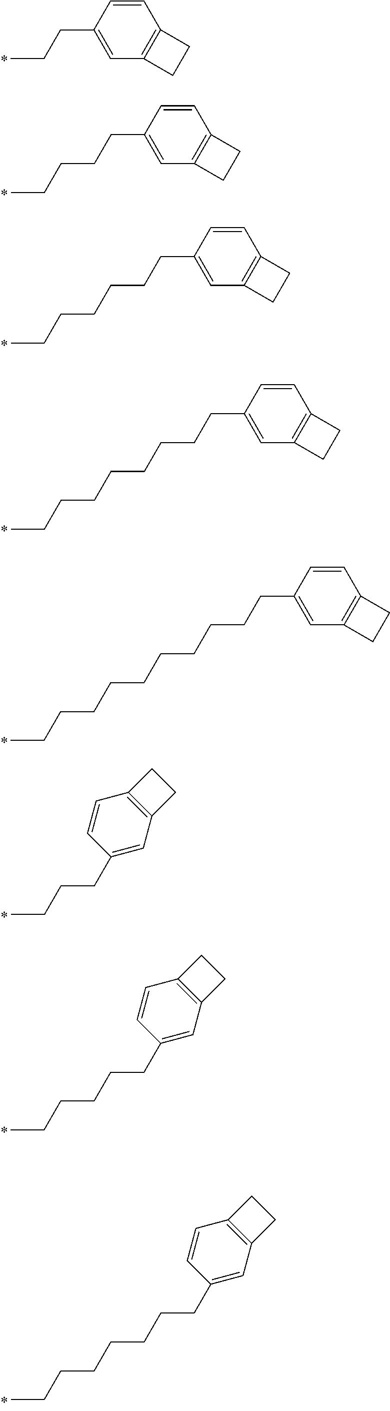

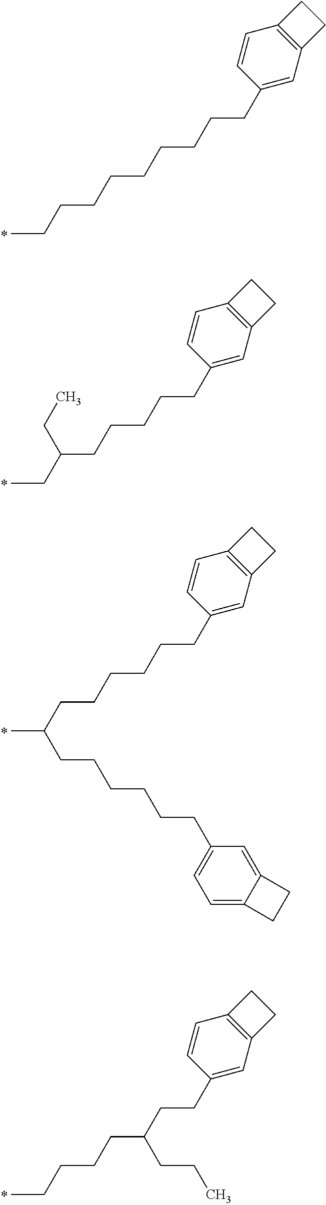

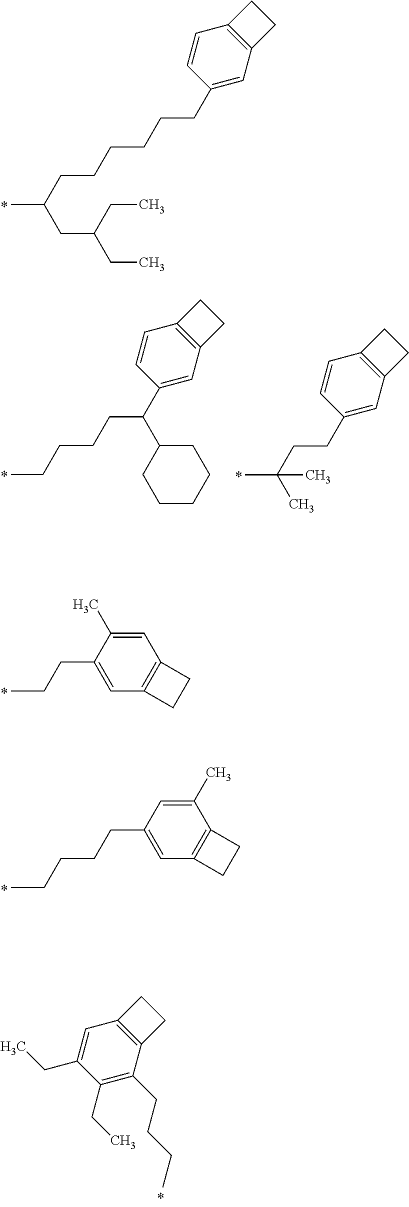

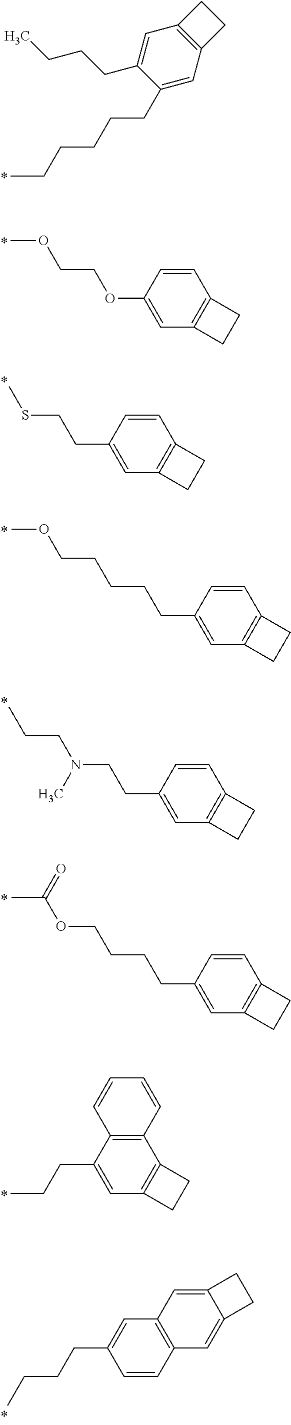

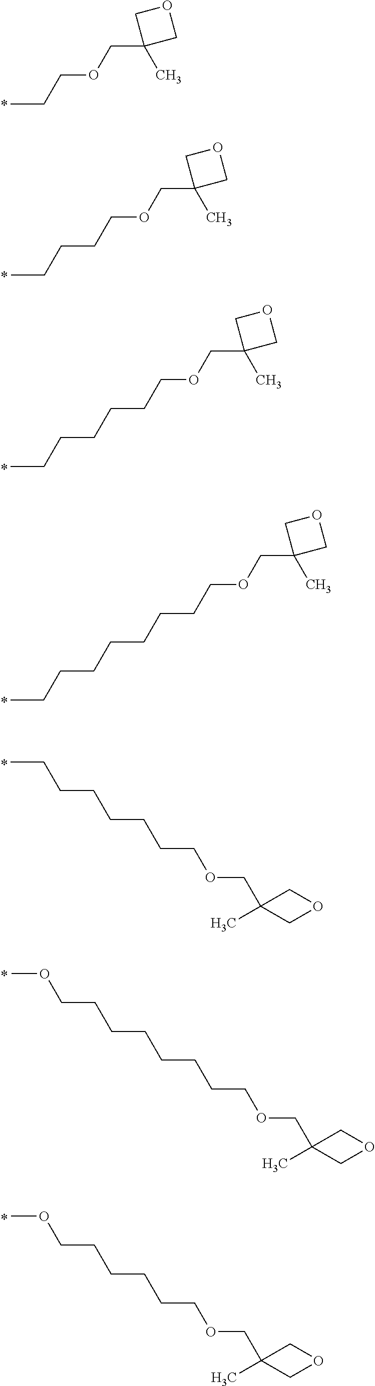

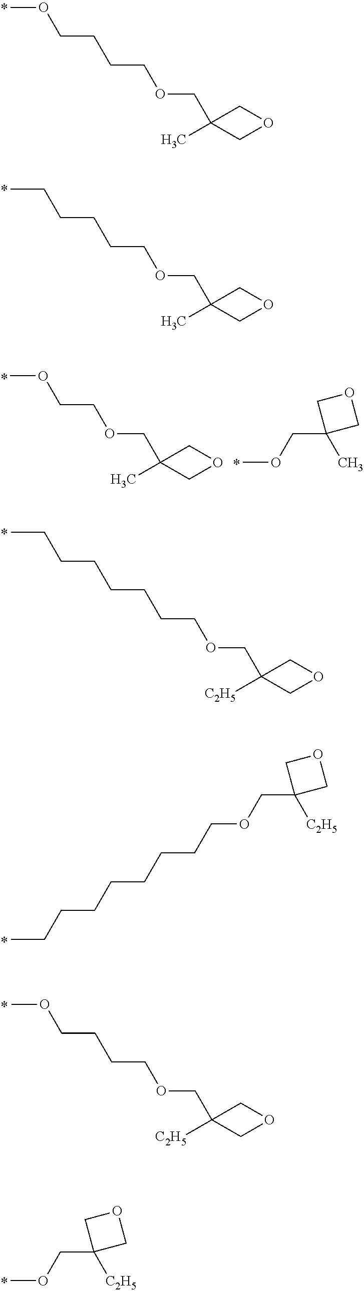

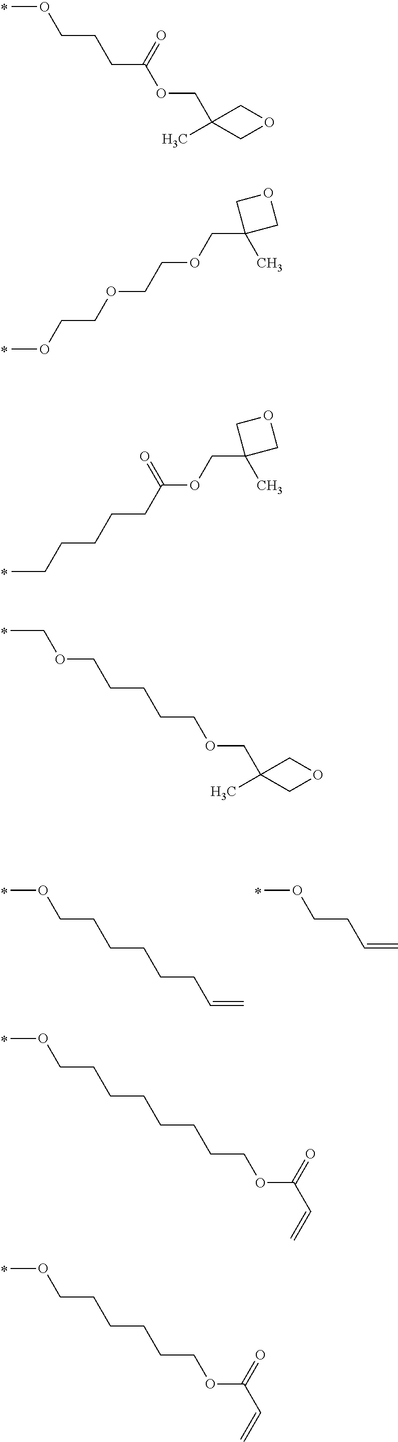

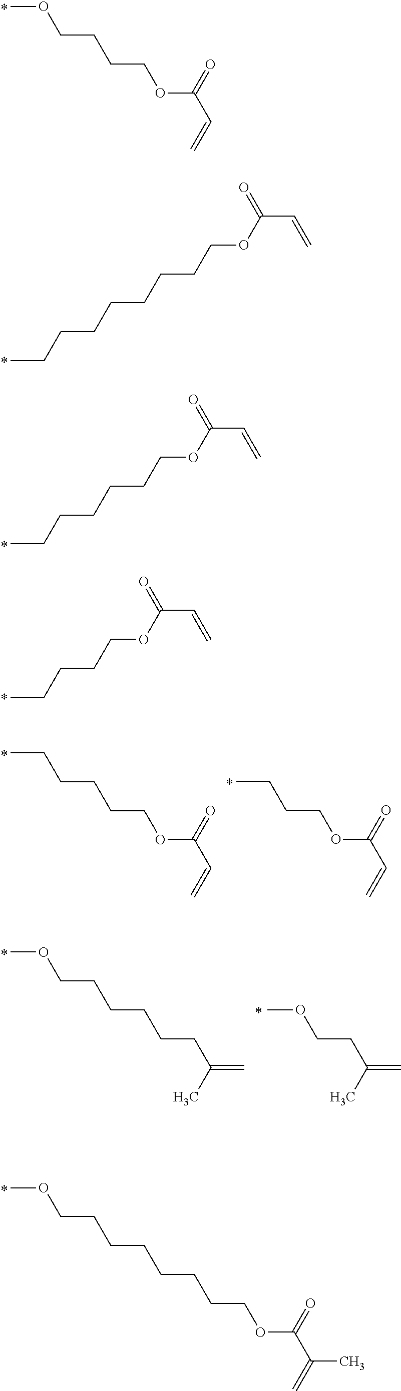

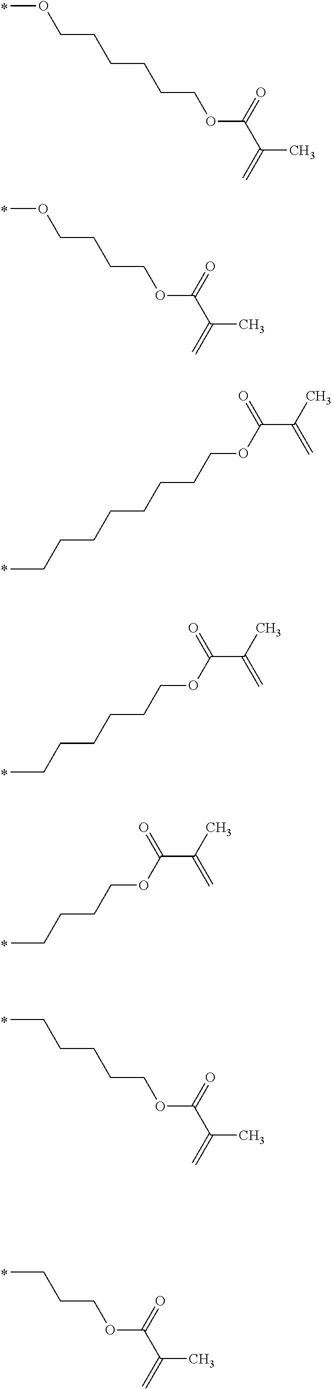

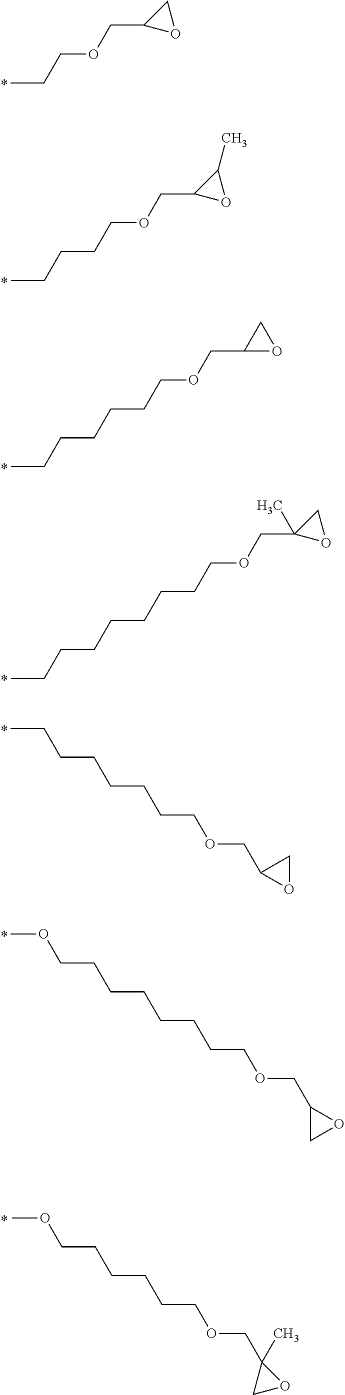

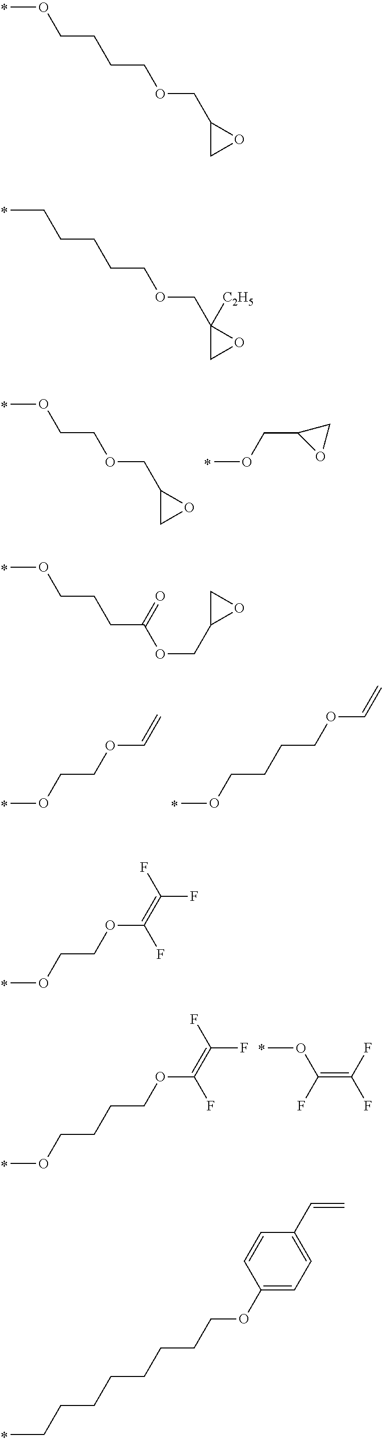

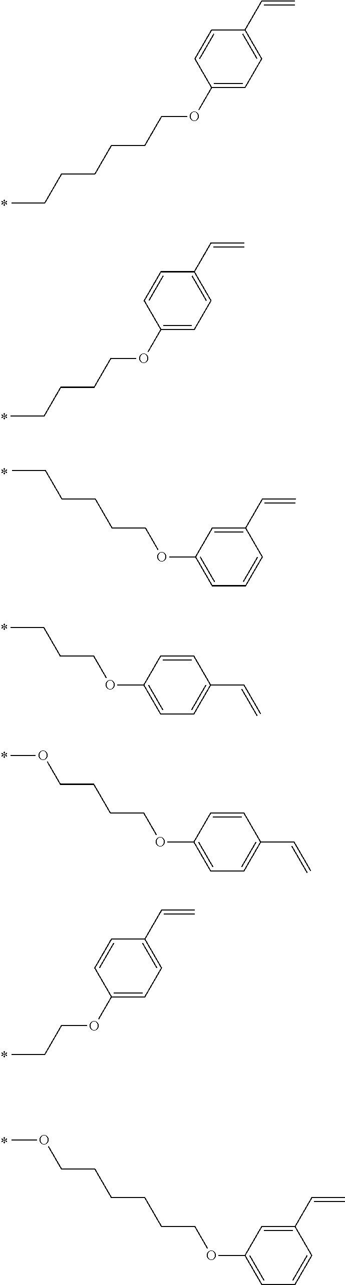

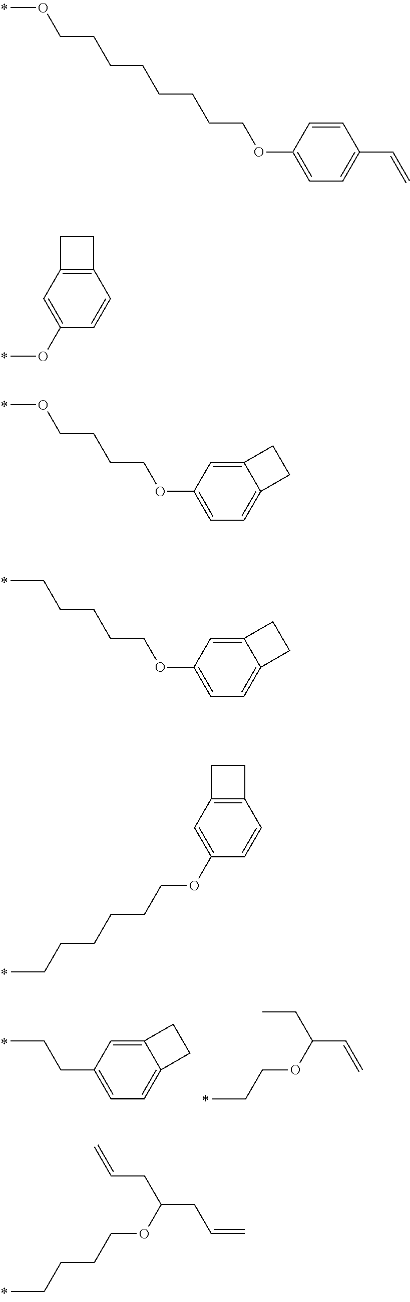

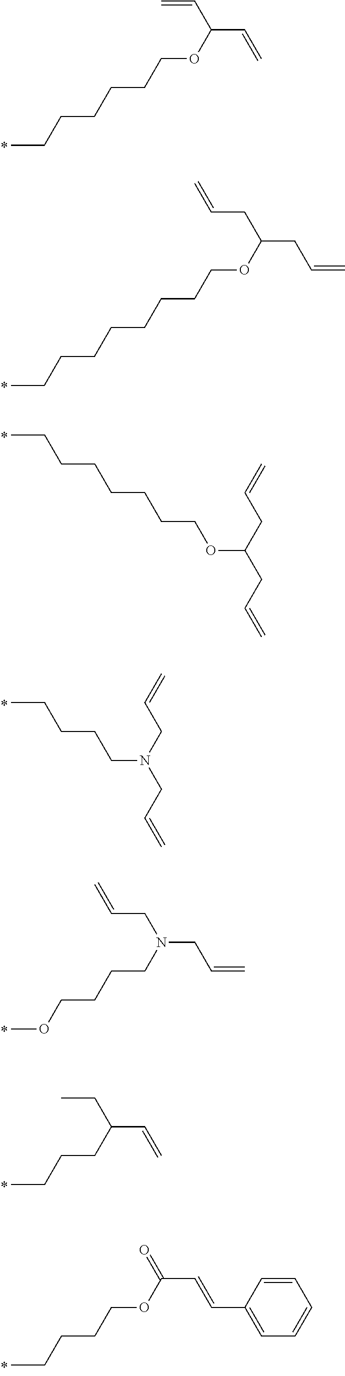

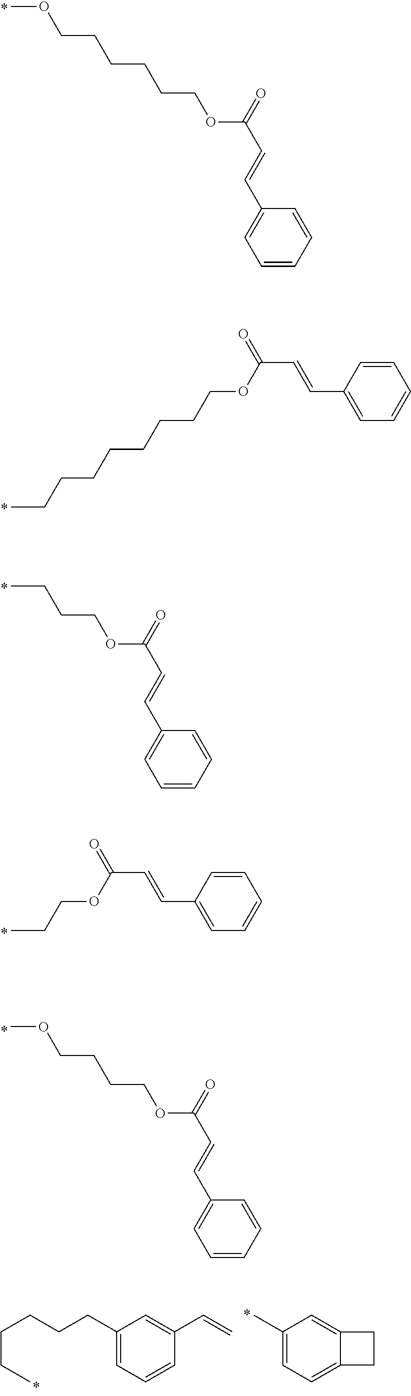

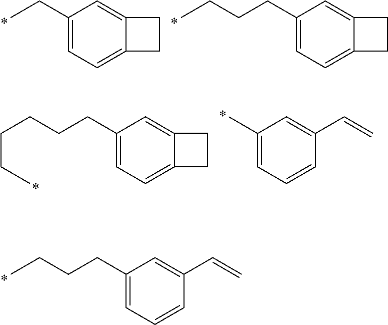

Preferred examples of R²⁰ are the same as those of R¹.

Examples of the structural unit (C) are shown below, although not limited thereto.

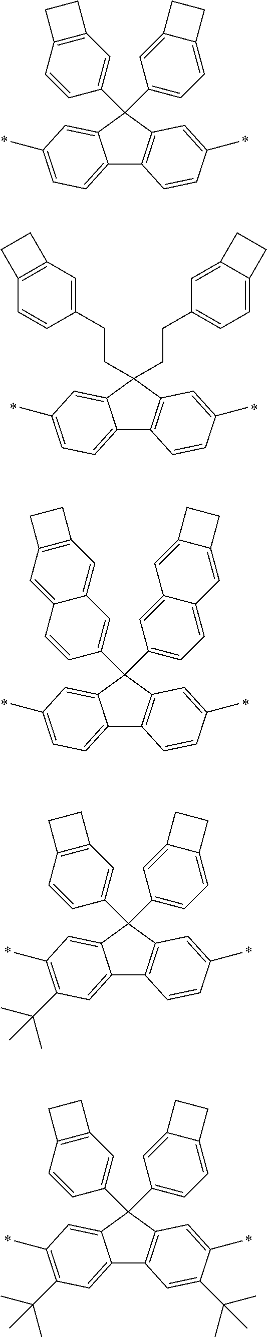

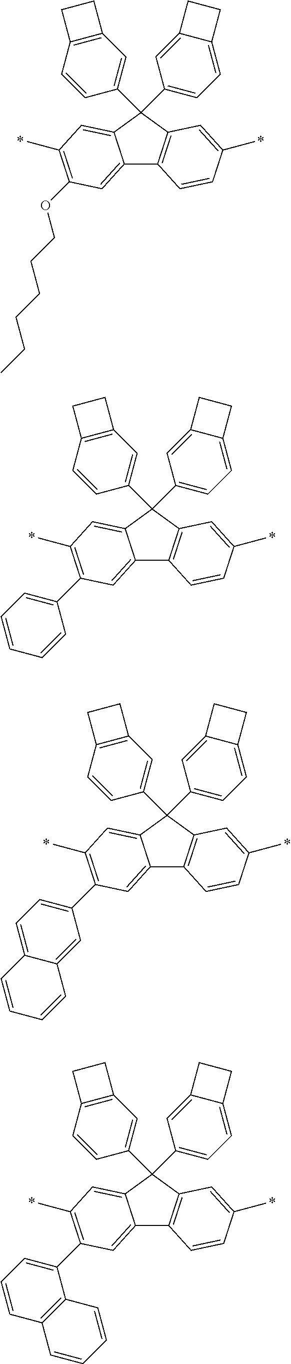

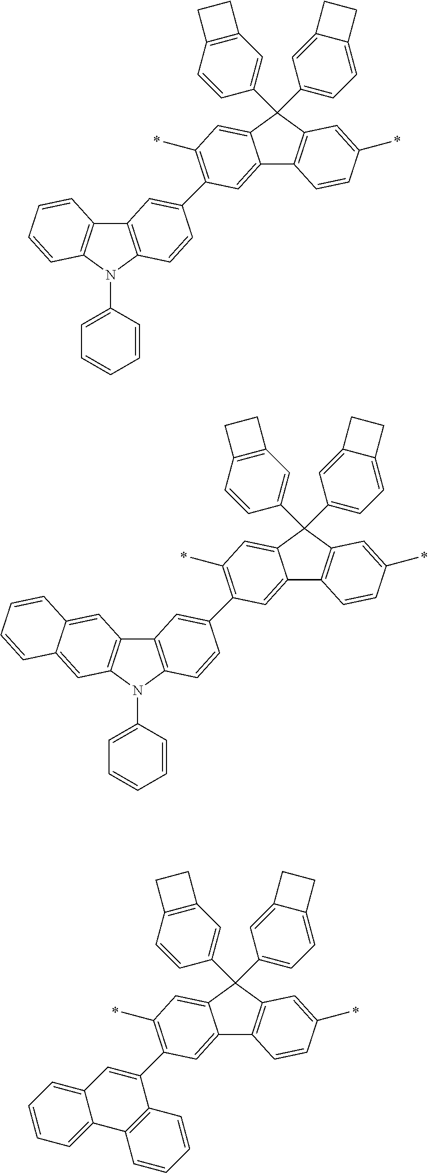

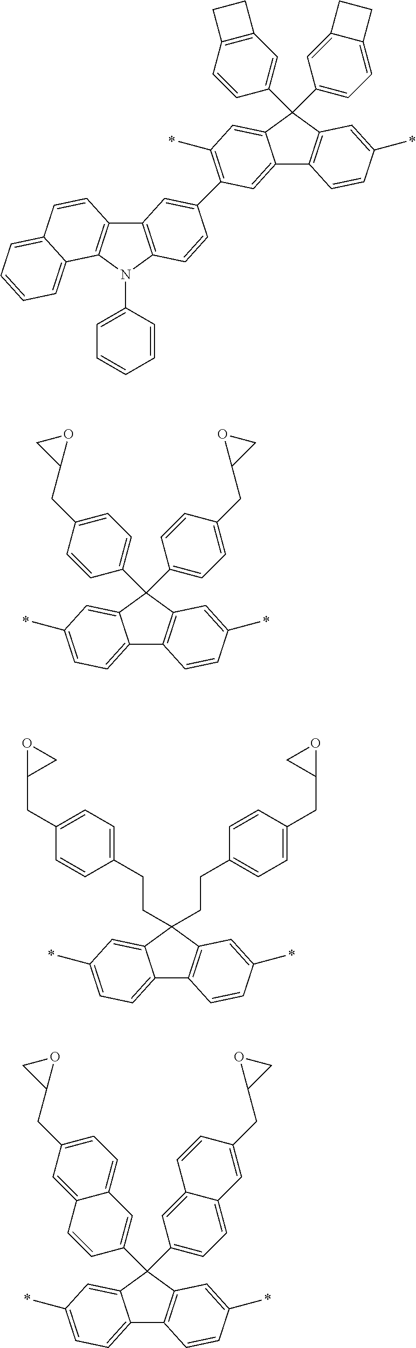

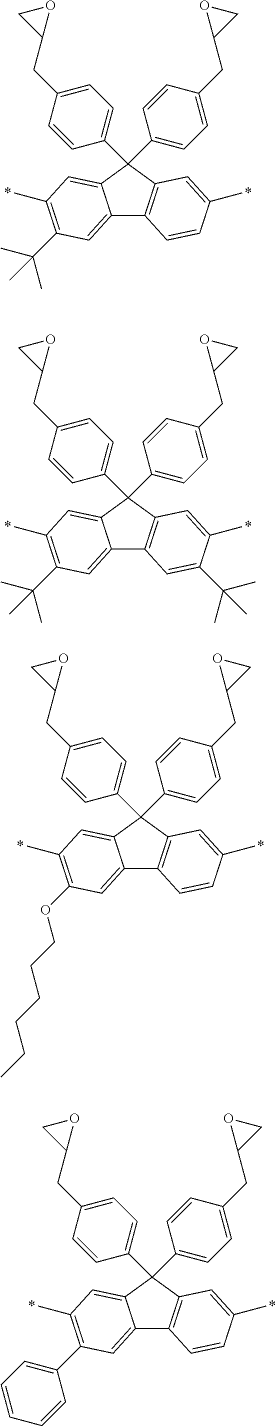

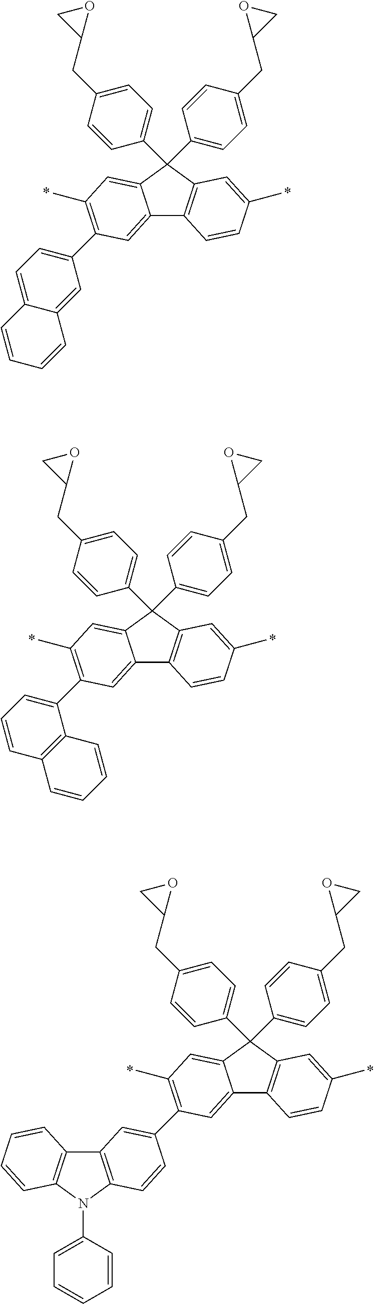

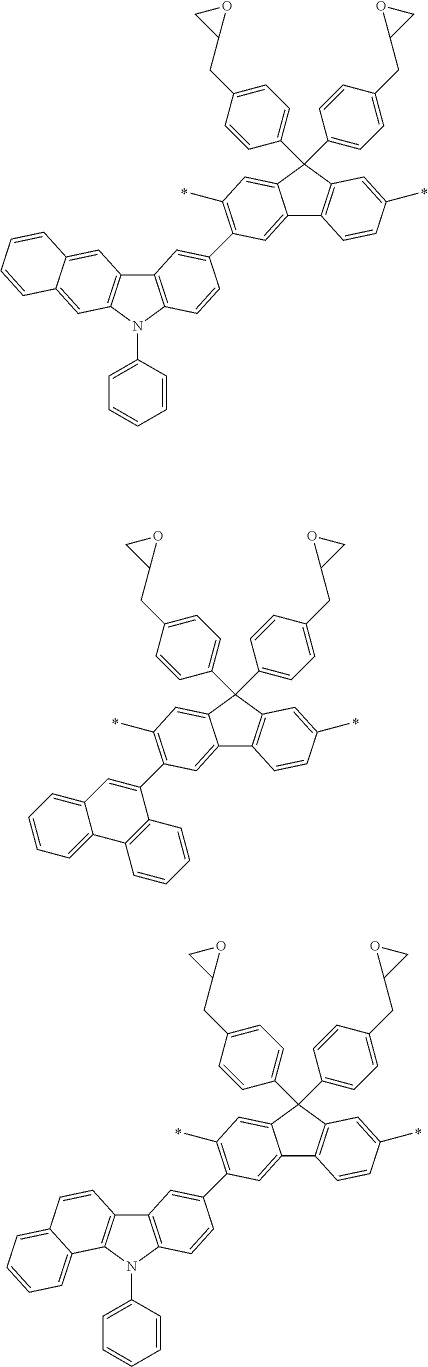

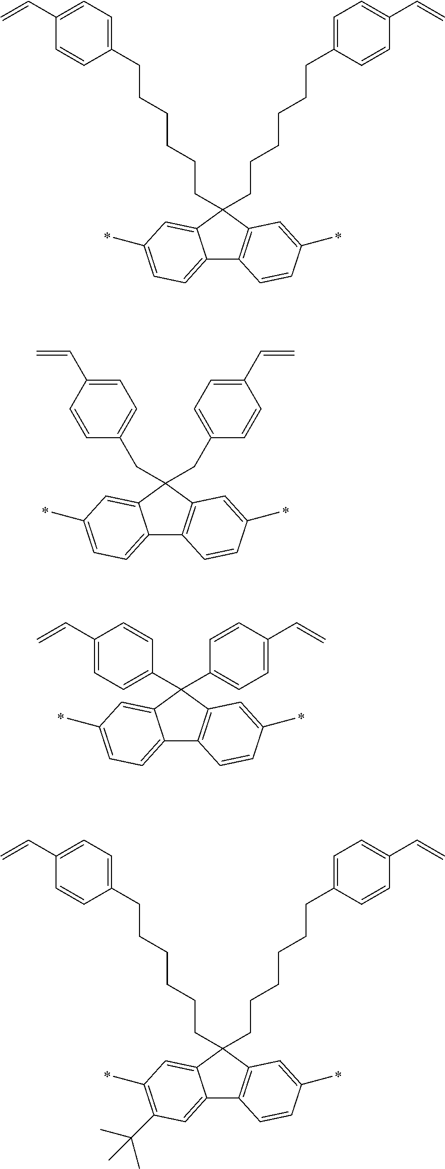

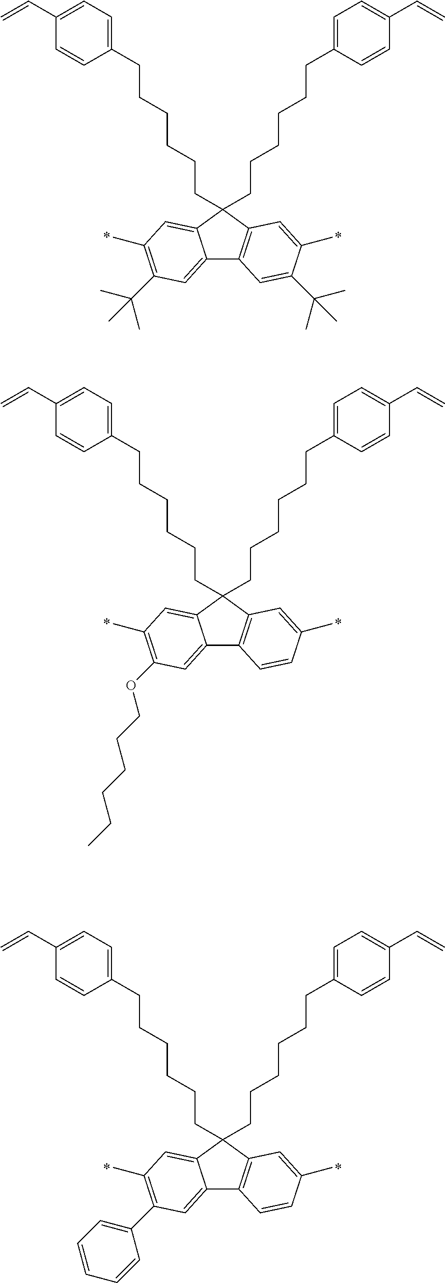

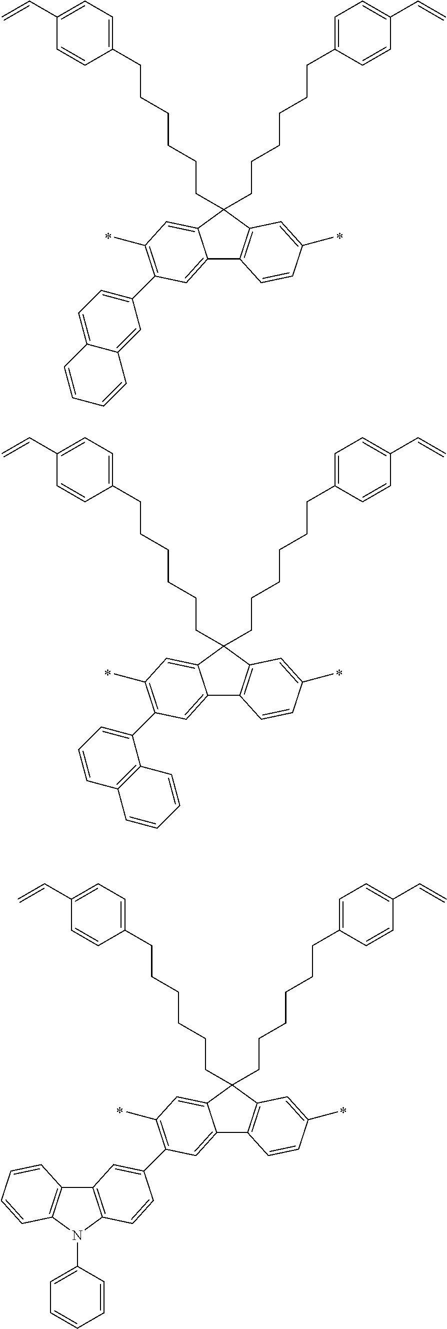

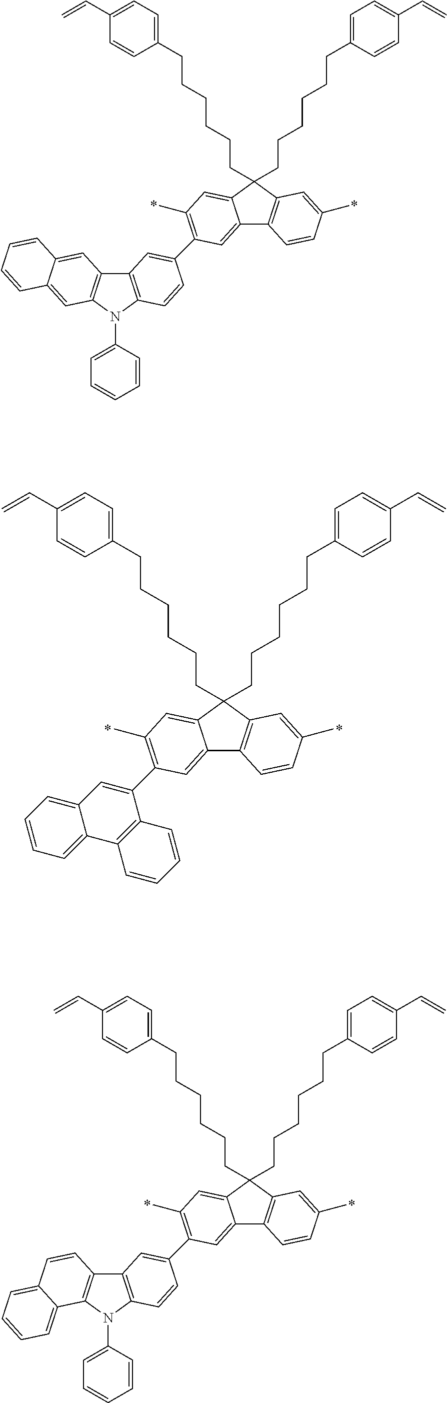

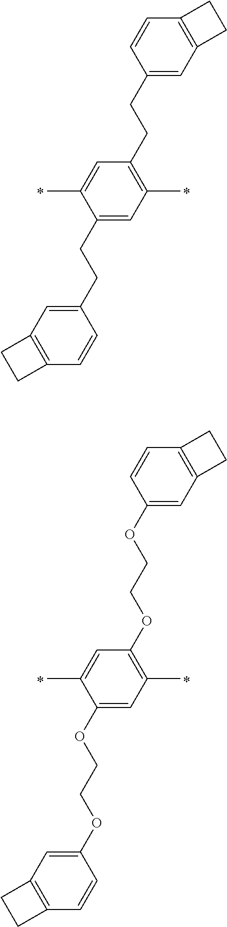

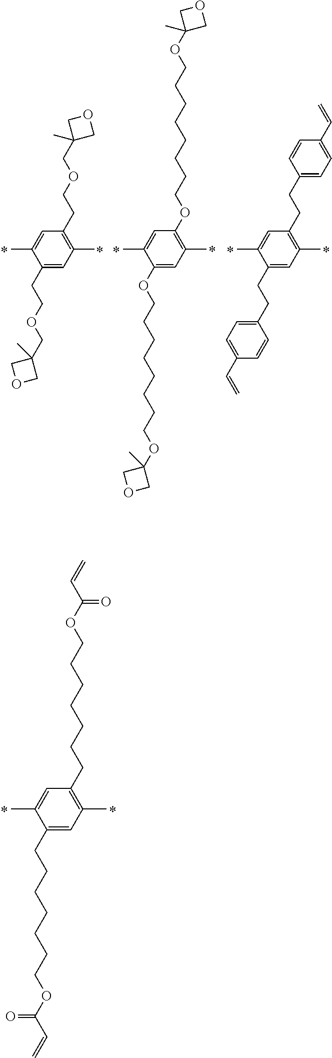

Of the structural units C exemplified above, more preferred are:

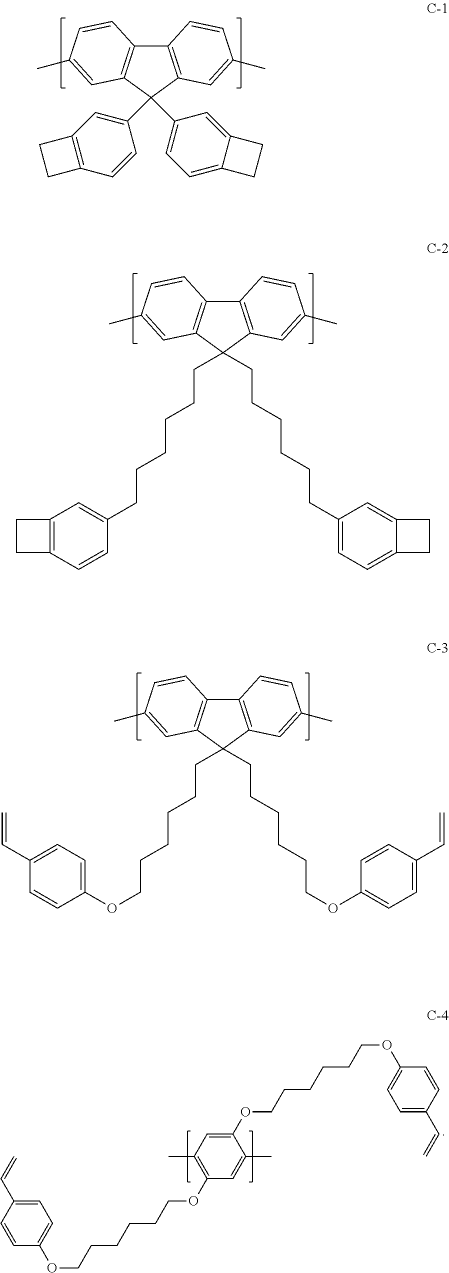

The type of the copolymerization structure of the copolymer of the invention is not limited and may be an alternating copolymer or a random copolymer.

The combinations of the repeating units of the cross-conjugated polymer are shown below.

TABLE 1

| | | | Ratio of units (mol %) | | |
|---|---|---|---|---|---|
| A | B | C | A | B | C |
| A-1 | B-1 | C-1 | 80 | 15 | 5 |
| A-2 | B-1 | C-1 | 80 | 15 | 5 |
| A-3 | B-1 | C-1 | 80 | 15 | 5 |
| A-4 | B-1 | C-1 | 80 | 15 | 5 |
| A-5 | B-1 | C-1 | 80 | 15 | 5 |
| A-6 | B-1 | C-1 | 80 | 15 | 5 |
| A-7 | B-1 | C-1 | 80 | 15 | 5 |
| A-8 | B-1 | C-1 | 80 | 15 | 5 |
| A-9 | B-1 | C-1 | 80 | 15 | 5 |
| A-10 | B-1 | C-1 | 80 | 15 | 5 |
| A-11 | B-1 | C-1 | 80 | 15 | 5 |
| A-12 | B-1 | C-1 | 80 | 15 | 5 |
| A-13 | B-1 | C-1 | 80 | 15 | 5 |
| A-14 | B-1 | C-1 | 80 | 15 | 5 |
| A-15 | B-1 | C-1 | 80 | 15 | 5 |
| A-16 | B-1 | C-1 | 80 | 15 | 5 |
| A-17 | B-1 | C-1 | 80 | 15 | 5 |
| A-18 | B-1 | C-1 | 80 | 15 | 5 |
| A-19 | B-1 | C-1 | 80 | 15 | 5 |
| A-20 | B-1 | C-1 | 80 | 15 | 5 |
| A-21 | B-1 | C-1 | 80 | 15 | 5 |
| A-22 | B-1 | C-1 | 80 | 15 | 5 |
| A-23 | B-1 | C-1 | 80 | 15 | 5 |
| A-24 | B-1 | C-1 | 80 | 15 | 5 |
| A-25 | B-1 | C-1 | 80 | 15 | 5 |
| A-26 | B-1 | C-1 | 80 | 15 | 5 |
| A-28 | B-1 | C-1 | 80 | 15 | 5 |
| A-29 | B-1 | C-1 | 80 | 15 | 5 |
| A-30 | B-1 | C-1 | 80 | 15 | 5 |
| A-31 | B-1 | C-1 | 80 | 15 | 5 |
| A-32 | B-1 | C-1 | 80 | 15 | 5 |
| A-33 | B-1 | C-1 | 80 | 15 | 5 |
| A-34 | B-1 | C-1 | 80 | 15 | 5 |
| A-35 | B-1 | C-1 | 80 | 15 | 5 |
| A-36 | B-1 | C-1 | 80 | 15 | 5 |
| A-37 | B-1 | C-1 | 80 | 15 | 5 |
| A-38 | B-1 | C-1 | 80 | 15 | 5 |
| A-39 | B-1 | C-1 | 80 | 15 | 5 |
| A-40 | B-1 | C-1 | 80 | 15 | 5 |
| A-41 | B-1 | C-1 | 80 | 15 | 5 |
| A-42 | B-1 | C-1 | 80 | 15 | 5 |
| A-43 | B-1 | C-1 | 80 | 15 | 5 |
| A-44 | B-1 | C-1 | 80 | 15 | 5 |
| A-45 | B-1 | C-1 | 80 | 15 | 5 |
| A-46 | B-1 | C-1 | 80 | 15 | 5 |
| A-47 | B-1 | C-1 | 80 | 15 | 5 |
| A-49 | B-1 | C-1 | 80 | 15 | 5 |
| A-50 | B-1 | C-1 | 80 | 15 | 5 |
| A-51 | B-1 | C-1 | 80 | 15 | 5 |
| A-52 | B-1 | C-1 | 80 | 15 | 5 |
| A-53 | B-1 | C-1 | 80 | 15 | 5 |
| A-54 | B-1 | C-1 | 80 | 15 | 5 |
| A-55 | B-1 | C-1 | 80 | 15 | 5 |
| A-56 | B-1 | C-1 | 80 | 15 | 5 |
| A-57 | B-1 | C-1 | 80 | 15 | 5 |
| A-58 | B-1 | C-1 | 80 | 15 | 5 |
| A-59 | B-1 | C-1 | 80 | 15 | 5 |
| A-60 | B-1 | C-1 | 80 | 15 | 5 |

TABLE 2

| | | | Ratio of units (mol %) | | |
|---|---|---|---|---|---|
| A | B | C | A | B | C |
| A-61 | B-1 | C-1 | 80 | 15 | 5 |
| A-62 | B-1 | C-1 | 80 | 15 | 5 |
| A-63 | B-1 | C-1 | 80 | 15 | 5 |
| A-64 | B-1 | C-1 | 80 | 15 | 5 |
| A-65 | B-1 | C-1 | 80 | 15 | 5 |
| A-66 | B-1 | C-1 | 80 | 15 | 5 |
| A-67 | B-1 | C-1 | 80 | 15 | 5 |
| A-68 | B-1 | C-1 | 80 | 15 | 5 |
| A-69 | B-1 | C-1 | 80 | 15 | 5 |
| A-70 | B-1 | C-1 | 80 | 15 | 5 |
| A-71 | B-1 | C-1 | 80 | 15 | 5 |
| A-72 | B-1 | C-1 | 80 | 15 | 5 |
| A-73 | B-1 | C-1 | 80 | 15 | 5 |
| A-74 | B-1 | C-1 | 80 | 15 | 5 |
| A-75 | B-1 | C-1 | 80 | 15 | 5 |
| A-76 | B-1 | C-1 | 80 | 15 | 5 |
| A-77 | B-1 | C-1 | 80 | 15 | 5 |
| A-78 | B-1 | C-1 | 80 | 15 | 5 |
| A-79 | B-1 | C-1 | 80 | 15 | 5 |
| A-80 | B-1 | C-1 | 80 | 15 | 5 |
| A-81 | B-1 | C-1 | 80 | 15 | 5 |
| A-82 | B-1 | C-1 | 80 | 15 | 5 |
| A-83 | B-1 | C-1 | 80 | 15 | 5 |
| A-84 | B-1 | C-1 | 80 | 15 | 5 |
| A-85 | B-1 | C-1 | 80 | 15 | 5 |
| A-86 | B-1 | C-1 | 80 | 15 | 5 |
| A-87 | B-1 | C-1 | 80 | 15 | 5 |
| A-88 | B-1 | C-1 | 80 | 15 | 5 |
| A-89 | B-1 | C-1 | 80 | 15 | 5 |
| A-90 | B-1 | C-1 | 80 | 15 | 5 |
| A-91 | B-1 | C-1 | 80 | 15 | 5 |
| A-92 | B-1 | C-1 | 80 | 15 | 5 |
| A-93 | B-1 | C-1 | 80 | 15 | 5 |
| A-94 | B-1 | C-1 | 80 | 15 | 5 |
| A-95 | B-1 | C-1 | 80 | 15 | 5 |
| A-96 | B-1 | C-1 | 80 | 15 | 5 |
| A-100 | B-1 | C-1 | 80 | 15 | 5 |
| A-101 | B-1 | C-1 | 80 | 15 | 5 |
| A-102 | B-1 | C-1 | 80 | 15 | 5 |
| A-103 | B-1 | C-1 | 80 | 15 | 5 |
| A-104 | B-1 | C-1 | 80 | 15 | 5 |
| A-105 | B-1 | C-1 | 80 | 15 | 5 |
| A-106 | B-1 | C-1 | 80 | 15 | 5 |
| A-107 | B-1 | C-1 | 80 | 15 | 5 |
| A-108 | B-1 | C-1 | 80 | 15 | 5 |
| A-109 | B-1 | C-1 | 80 | 15 | 5 |
| A-110 | B-1 | C-1 | 80 | 15 | 5 |
| A-111 | B-1 | C-1 | 80 | 15 | 5 |
| A-112 | B-1 | C-1 | 80 | 15 | 5 |
| A-113 | B-1 | C-1 | 80 | 15 | 5 |
| A-114 | B-1 | C-1 | 80 | 15 | 5 |
| A-115 | B-1 | C-1 | 80 | 15 | 5 |
| A-116 | B-1 | C-1 | 80 | 15 | 5 |
| A-117 | B-1 | C-1 | 80 | 15 | 5 |
| A-118 | B-1 | C-1 | 80 | 15 | 5 |
| A-119 | B-1 | C-1 | 80 | 15 | 5 |
| A-120 | B-1 | C-1 | 80 | 15 | 5 |
| A-121 | B-1 | C-1 | 80 | 15 | 5 |
| A-122 | B-1 | C-1 | 80 | 15 | 5 |
| A-123 | B-1 | C-1 | 80 | 15 | 5 |

TABLE 3

| | | | Ratio of units (mol %) | | |
|---|---|---|---|---|---|
| A | B | C | A | B | C |
| A-124 | B-1 | C-1 | 80 | 15 | 5 |
| A-125 | B-1 | C-1 | 80 | 15 | 5 |
| A-126 | B-1 | C-1 | 80 | 15 | 5 |
| A-127 | B-1 | C-1 | 80 | 15 | 5 |
| A-128 | B-1 | C-1 | 80 | 15 | 5 |
| A-129 | B-1 | C-1 | 80 | 15 | 5 |
| A-130 | B-1 | C-1 | 80 | 15 | 5 |
| A-131 | B-1 | C-1 | 80 | 15 | 5 |
| A-132 | B-1 | C-1 | 80 | 15 | 5 |
| A-133 | B-1 | C-1 | 80 | 15 | 5 |
| A-134 | B-1 | C-1 | 80 | 15 | 5 |
| A-135 | B-1 | C-1 | 80 | 15 | 5 |
| A-136 | B-1 | C-1 | 80 | 15 | 5 |
| A-137 | B-1 | C-1 | 80 | 15 | 5 |
| A-138 | B-1 | C-1 | 80 | 15 | 5 |
| A-139 | B-1 | C-1 | 80 | 15 | 5 |
| A-140 | B-1 | C-1 | 80 | 15 | 5 |
| A-141 | B-1 | C-1 | 80 | 15 | 5 |
| A-142 | B-1 | C-1 | 80 | 15 | 5 |
| A-143 | B-1 | C-1 | 80 | 15 | 5 |
| A-144 | B-1 | C-1 | 80 | 15 | 5 |
| A-145 | B-1 | C-1 | 80 | 15 | 5 |
| A-146 | B-1 | C-1 | 80 | 15 | 5 |
| A-147 | B-1 | C-1 | 80 | 15 | 5 |
| A-160 | B-1 | C-1 | 80 | 15 | 5 |
| A-161 | B-1 | C-1 | 80 | 15 | 5 |
| A-162 | B-1 | C-1 | 80 | 15 | 5 |
| A-163 | B-1 | C-1 | 80 | 15 | 5 |
| A-164 | B-1 | C-1 | 80 | 15 | 5 |
| A-165 | B-1 | C-1 | 80 | 15 | 5 |
| A-166 | B-1 | C-1 | 80 | 15 | 5 |
| A-167 | B-1 | C-1 | 80 | 15 | 5 |
| A-168 | B-1 | C-1 | 80 | 15 | 5 |
| A-169 | B-1 | C-1 | 80 | 15 | 5 |
| A-170 | B-1 | C-1 | 80 | 15 | 5 |
| A-171 | B-1 | C-1 | 80 | 15 | 5 |
| A-172 | B-1 | C-1 | 80 | 15 | 5 |
| A-173 | B-1 | C-1 | 80 | 15 | 5 |
| A-174 | B-1 | C-1 | 80 | 15 | 5 |
| A-175 | B-1 | C-1 | 80 | 15 | 5 |
| A-176 | B-1 | C-1 | 80 | 15 | 5 |
| A-177 | B-1 | C-1 | 80 | 15 | 5 |
| A-178 | B-1 | C-1 | 80 | 15 | 5 |
| A-179 | B-1 | C-1 | 80 | 15 | 5 |
| A-180 | B-1 | C-1 | 80 | 15 | 5 |
| A-181 | B-1 | C-1 | 80 | 15 | 5 |
| A-182 | B-1 | C-1 | 80 | 15 | 5 |
| A-183 | B-1 | C-1 | 80 | 15 | 5 |
| A-190 | B-1 | C-1 | 80 | 15 | 5 |
| A-191 | B-1 | C-1 | 80 | 15 | 5 |
| A-192 | B-1 | C-1 | 80 | 15 | 5 |
| A-193 | B-1 | C-1 | 80 | 15 | 5 |
| A-194 | B-1 | C-1 | 80 | 15 | 5 |
| A-195 | B-1 | C-1 | 80 | 15 | 5 |
| A-196 | B-1 | C-1 | 80 | 15 | 5 |
| A-197 | B-1 | C-1 | 80 | 15 | 5 |
| A-198 | B-1 | C-1 | 80 | 15 | 5 |
| A-199 | B-1 | C-1 | 80 | 15 | 5 |
| A-200 | B-1 | C-1 | 80 | 15 | 5 |
| A-201 | B-1 | C-1 | 80 | 15 | 5 |
| A-202 | B-1 | C-1 | 80 | 15 | 5 |
| A-203 | B-1 | C-1 | 80 | 15 | 5 |
| A-204 | B-1 | C-1 | 80 | 15 | 5 |
| A-205 | B-1 | C-1 | 80 | 15 | 5 |
| A-206 | B-1 | C-1 | 80 | 15 | 5 |
| A-207 | B-1 | C-1 | 80 | 15 | 5 |
| A-208 | B-1 | C-1 | 80 | 15 | 5 |
| A-209 | B-1 | C-1 | 80 | 15 | 5 |
| A-210 | B-1 | C-1 | 80 | 15 | 5 |
| A-211 | B-1 | C-1 | 80 | 15 | 5 |
| A-212 | B-1 | C-1 | 80 | 15 | 5 |
| A-213 | B-1 | C-1 | 80 | 15 | 5 |

TABLE 4

| | | | Ratio of units (mol %) | | |
|---|---|---|---|---|---|
| A | B | C | A | B | C |
| A-220 | B-1 | C-1 | 80 | 15 | 5 |
| A-221 | B-1 | C-1 | 80 | 15 | 5 |
| A-222 | B-1 | C-1 | 80 | 15 | 5 |
| A-223 | B-1 | C-1 | 80 | 15 | 5 |
| A-224 | B-1 | C-1 | 80 | 15 | 5 |
| A-225 | B-1 | C-1 | 80 | 15 | 5 |
| A-226 | B-1 | C-1 | 80 | 15 | 5 |
| A-227 | B-1 | C-1 | 80 | 15 | 5 |
| A-228 | B-1 | C-1 | 80 | 15 | 5 |
| A-229 | B-1 | C-1 | 80 | 15 | 5 |
| A-230 | B-1 | C-1 | 80 | 15 | 5 |
| A-231 | B-1 | C-1 | 80 | 15 | 5 |
| A-232 | B-1 | C-1 | 80 | 15 | 5 |
| A-233 | B-1 | C-1 | 80 | 15 | 5 |
| A-234 | B-1 | C-1 | 80 | 15 | 5 |
| A-235 | B-1 | C-1 | 80 | 15 | 5 |
| A-236 | B-1 | C-1 | 80 | 15 | 5 |
| A-237 | B-1 | C-1 | 80 | 15 | 5 |
| A-238 | B-1 | C-1 | 80 | 15 | 5 |
| A-239 | B-1 | C-1 | 80 | 15 | 5 |
| A-240 | B-1 | C-1 | 80 | 15 | 5 |

TABLE 5

| | | | Ratio of | | |
|---|---|---|---|---|---|
| | | | units | | |
| | | | (mol %) | | |
| A | B | C | A | B | C |
| A-1 | B-1 | C-2 | 80 | 15 | 5 |
| A-2 | B-1 | C-2 | 80 | 15 | 5 |
| A-3 | B-1 | C-2 | 80 | 15 | 5 |
| A-4 | B-1 | C-2 | 80 | 15 | 5 |
| A-5 | B-1 | C-2 | 80 | 15 | 5 |
| A-6 | B-1 | C-2 | 80 | 15 | 5 |
| A-7 | B-1 | C-2 | 80 | 15 | 5 |
| A-8 | B-1 | C-2 | 80 | 15 | 5 |
| A-9 | B-1 | C-2 | 80 | 15 | 5 |
| A-10 | B-1 | C-2 | 80 | 15 | 5 |
| A-11 | B-1 | C-2 | 80 | 15 | 5 |
| A-12 | B-1 | C-2 | 80 | 15 | 5 |
| A-13 | B-1 | C-2 | 80 | 15 | 5 |
| A-14 | B-1 | C-2 | 80 | 15 | 5 |
| A-15 | B-1 | C-2 | 80 | 15 | 5 |
| A-16 | B-1 | C-2 | 80 | 15 | 5 |
| A-17 | B-1 | C-2 | 80 | 15 | 5 |
| A-18 | B-1 | C-2 | 80 | 15 | 5 |
| A-19 | B-1 | C-2 | 80 | 15 | 5 |
| A-20 | B-1 | C-2 | 80 | 15 | 5 |
| A-21 | B-1 | C-2 | 80 | 15 | 5 |
| A-22 | B-1 | C-2 | 80 | 15 | 5 |
| A-23 | B-1 | C-2 | 80 | 15 | 5 |
| A-24 | B-1 | C-2 | 80 | 15 | 5 |
| A-25 | B-1 | C-2 | 80 | 15 | 5 |
| A-26 | B-1 | C-2 | 80 | 15 | 5 |
| A-28 | B-1 | C-2 | 80 | 15 | 5 |
| A-29 | B-1 | C-2 | 80 | 15 | 5 |
| A-30 | B-1 | C-2 | 80 | 15 | 5 |
| A-31 | B-1 | C-2 | 80 | 15 | 5 |
| A-32 | B-1 | C-2 | 80 | 15 | 5 |
| A-33 | B-1 | C-2 | 80 | 15 | 5 |
| A-34 | B-1 | C-2 | 80 | 15 | 5 |
| A-35 | B-1 | C-2 | 80 | 15 | 5 |
| A-36 | B-1 | C-2 | 80 | 15 | 5 |
| A-37 | B-1 | C-2 | 80 | 15 | 5 |
| A-38 | B-1 | C-2 | 80 | 15 | 5 |
| A-39 | B-1 | C-2 | 80 | 15 | 5 |
| A-40 | B-1 | C-2 | 80 | 15 | 5 |
| A-41 | B-1 | C-2 | 80 | 15 | 5 |
| A-42 | B-1 | C-2 | 80 | 15 | 5 |
| A-43 | B-1 | C-2 | 80 | 15 | 5 |
| A-44 | B-1 | C-2 | 80 | 15 | 5 |
| A-45 | B-1 | C-2 | 80 | 15 | 5 |
| A-46 | B-1 | C-2 | 80 | 15 | 5 |
| A-47 | B-1 | C-2 | 80 | 15 | 5 |
| A-49 | B-1 | C-2 | 80 | 15 | 5 |
| A-50 | B-1 | C-2 | 80 | 15 | 5 |
| A-51 | B-1 | C-2 | 80 | 15 | 5 |
| A-52 | B-1 | C-2 | 80 | 15 | 5 |
| A-53 | B-1 | C-2 | 80 | 15 | 5 |
| A-54 | B-1 | C-2 | 80 | 15 | 5 |
| A-55 | B-1 | C-2 | 80 | 15 | 5 |
| A-56 | B-1 | C-2 | 80 | 15 | 5 |
| A-57 | B-1 | C-2 | 80 | 15 | 5 |
| A-58 | B-1 | C-2 | 80 | 15 | 5 |
| A-59 | B-1 | C-2 | 80 | 15 | 5 |
| A-60 | B-1 | C-2 | 80 | 15 | 5 |

TABLE 6

| | | | Ratio of | | |
|---|---|---|---|---|---|
| | | | units | | |
| | | | (mol %) | | |
| A | B | C | A | B | C |
| A-61 | B-1 | C-2 | 80 | 15 | 5 |
| A-62 | B-1 | C-2 | 80 | 15 | 5 |
| A-63 | B-1 | C-2 | 80 | 15 | 5 |
| A-64 | B-1 | C-2 | 80 | 15 | 5 |
| A-65 | B-1 | C-2 | 80 | 15 | 5 |
| A-66 | B-1 | C-2 | 80 | 15 | 5 |
| A-67 | B-1 | C-2 | 80 | 15 | 5 |
| A-68 | B-1 | C-2 | 80 | 15 | 5 |
| A-69 | B-1 | C-2 | 80 | 15 | 5 |
| A-70 | B-1 | C-2 | 80 | 15 | 5 |
| A-71 | B-1 | C-2 | 80 | 15 | 5 |
| A-72 | B-1 | C-2 | 80 | 15 | 5 |
| A-73 | B-1 | C-2 | 80 | 15 | 5 |
| A-74 | B-1 | C-2 | 80 | 15 | 5 |
| A-75 | B-1 | C-2 | 80 | 15 | 5 |
| A-76 | B-1 | C-2 | 80 | 15 | 5 |
| A-77 | B-1 | C-2 | 80 | 15 | 5 |
| A-78 | B-1 | C-2 | 80 | 15 | 5 |
| A-79 | B-1 | C-2 | 80 | 15 | 5 |
| A-80 | B-1 | C-2 | 80 | 15 | 5 |
| A-81 | B-1 | C-2 | 80 | 15 | 5 |
| A-82 | B-1 | C-2 | 80 | 15 | 5 |
| A-83 | B-1 | C-2 | 80 | 15 | 5 |
| A-84 | B-1 | C-2 | 80 | 15 | 5 |
| A-85 | B-1 | C-2 | 80 | 15 | 5 |
| A-86 | B-1 | C-2 | 80 | 15 | 5 |
| A-87 | B-1 | C-2 | 80 | 15 | 5 |
| A-88 | B-1 | C-2 | 80 | 15 | 5 |
| A-89 | B-1 | C-2 | 80 | 15 | 5 |
| A-90 | B-1 | C-2 | 80 | 15 | 5 |
| A-91 | B-1 | C-2 | 80 | 15 | 5 |
| A-92 | B-1 | C-2 | 80 | 15 | 5 |
| A-93 | B-1 | C-2 | 80 | 15 | 5 |
| A-94 | B-1 | C-2 | 80 | 15 | 5 |
| A-95 | B-1 | C-2 | 80 | 15 | 5 |
| A-96 | B-1 | C-2 | 80 | 15 | 5 |
| A-100 | B-1 | C-2 | 80 | 15 | 5 |
| A-101 | B-1 | C-2 | 80 | 15 | 5 |
| A-102 | B-1 | C-2 | 80 | 15 | 5 |
| A-103 | B-1 | C-2 | 80 | 15 | 5 |
| A-104 | B-1 | C-2 | 80 | 15 | 5 |
| A-105 | B-1 | C-2 | 80 | 15 | 5 |
| A-106 | B-1 | C-2 | 80 | 15 | 5 |
| A-107 | B-1 | C-2 | 80 | 15 | 5 |
| A-108 | B-1 | C-2 | 80 | 15 | 5 |
| A-109 | B-1 | C-2 | 80 | 15 | 5 |
| A-110 | B-1 | C-2 | 80 | 15 | 5 |
| A-111 | B-1 | C-2 | 80 | 15 | 5 |
| A-112 | B-1 | C-2 | 80 | 15 | 5 |
| A-113 | B-1 | C-2 | 80 | 15 | 5 |
| A-114 | B-1 | C-2 | 80 | 15 | 5 |
| A-115 | B-1 | C-2 | 80 | 15 | 5 |
| A-116 | B-1 | C-2 | 80 | 15 | 5 |
| A-117 | B-1 | C-2 | 80 | 15 | 5 |
| A-118 | B-1 | C-2 | 80 | 15 | 5 |
| A-119 | B-1 | C-2 | 80 | 15 | 5 |
| A-120 | B-1 | C-2 | 80 | 15 | 5 |
| A-121 | B-1 | C-2 | 80 | 15 | 5 |
| A-122 | B-1 | C-2 | 80 | 15 | 5 |
| A-123 | B-1 | C-2 | 80 | 15 | 5 |

TABLE 7

| | | | Ratio of | | |
|---|---|---|---|---|---|
| | | | units | | |
| | | | (mol %) | | |
| A | B | C | A | B | C |
| A-124 | B-1 | C-2 | 80 | 15 | 5 |
| A-125 | B-1 | C-2 | 80 | 15 | 5 |
| A-126 | B-1 | C-2 | 80 | 15 | 5 |
| A-127 | B-1 | C-2 | 80 | 15 | 5 |
| A-128 | B-1 | C-2 | 80 | 15 | 5 |
| A-129 | B-1 | C-2 | 80 | 15 | 5 |
| A-130 | B-1 | C-2 | 80 | 15 | 5 |
| A-131 | B-1 | C-2 | 80 | 15 | 5 |
| A-132 | B-1 | C-2 | 80 | 15 | 5 |
| A-133 | B-1 | C-2 | 80 | 15 | 5 |
| A-134 | B-1 | C-2 | 80 | 15 | 5 |
| A-135 | B-1 | C-2 | 80 | 15 | 5 |
| A-136 | B-1 | C-2 | 80 | 15 | 5 |
| A-137 | B-1 | C-2 | 80 | 15 | 5 |
| A-138 | B-1 | C-2 | 80 | 15 | 5 |
| A-139 | B-1 | C-2 | 80 | 15 | 5 |
| A-140 | B-1 | C-2 | 80 | 15 | 5 |
| A-141 | B-1 | C-2 | 80 | 15 | 5 |
| A-142 | B-1 | C-2 | 80 | 15 | 5 |
| A-143 | B-1 | C-2 | 80 | 15 | 5 |
| A-144 | B-1 | C-2 | 80 | 15 | 5 |
| A-145 | B-1 | C-2 | 80 | 15 | 5 |
| A-146 | B-1 | C-2 | 80 | 15 | 5 |
| A-147 | B-1 | C-2 | 80 | 15 | 5 |
| A-160 | B-1 | C-2 | 80 | 15 | 5 |
| A-161 | B-1 | C-2 | 80 | 15 | 5 |
| A-162 | B-1 | C-2 | 80 | 15 | 5 |
| A-163 | B-1 | C-2 | 80 | 15 | 5 |
| A-164 | B-1 | C-2 | 80 | 15 | 5 |
| A-165 | B-1 | C-2 | 80 | 15 | 5 |
| A-166 | B-1 | C-2 | 80 | 15 | 5 |
| A-167 | B-1 | C-2 | 80 | 15 | 5 |
| A-168 | B-1 | C-2 | 80 | 15 | 5 |
| A-169 | B-1 | C-2 | 80 | 15 | 5 |
| A-170 | B-1 | C-2 | 80 | 15 | 5 |
| A-171 | B-1 | C-2 | 80 | 15 | 5 |
| A-172 | B-1 | C-2 | 80 | 15 | 5 |
| A-173 | B-1 | C-2 | 80 | 15 | 5 |
| A-174 | B-1 | C-2 | 80 | 15 | 5 |
| A-175 | B-1 | C-2 | 80 | 15 | 5 |
| A-176 | B-1 | C-2 | 80 | 15 | 5 |
| A-177 | B-1 | C-2 | 80 | 15 | 5 |
| A-178 | B-1 | C-2 | 80 | 15 | 5 |
| A-179 | B-1 | C-2 | 80 | 15 | 5 |
| A-180 | B-1 | C-2 | 80 | 15 | 5 |
| A-181 | B-1 | C-2 | 80 | 15 | 5 |
| A-182 | B-1 | C-2 | 80 | 15 | 5 |
| A-183 | B-1 | C-2 | 80 | 15 | 5 |
| A-190 | B-1 | C-2 | 80 | 15 | 5 |
| A-191 | B-1 | C-2 | 80 | 15 | 5 |
| A-192 | B-1 | C-2 | 80 | 15 | 5 |
| A-193 | B-1 | C-2 | 80 | 15 | 5 |
| A-194 | B-1 | C-2 | 80 | 15 | 5 |
| A-195 | B-1 | C-2 | 80 | 15 | 5 |
| A-196 | B-1 | C-2 | 80 | 15 | 5 |
| A-197 | B-1 | C-2 | 80 | 15 | 5 |
| A-198 | B-1 | C-2 | 80 | 15 | 5 |
| A-199 | B-1 | C-2 | 80 | 15 | 5 |

TABLE 8

| | | | Ratio | | |
|---|---|---|---|---|---|
| | | | of units | | |
| A | B | C | A | B | C |
| A-200 | B-1 | C-2 | 80 | 15 | 5 |
| A-201 | B-1 | C-2 | 80 | 15 | 5 |
| A-202 | B-1 | C-2 | 80 | 15 | 5 |
| A-203 | B-1 | C-2 | 80 | 15 | 5 |
| A-204 | B-1 | C-2 | 80 | 15 | 5 |
| A-205 | B-1 | C-2 | 80 | 15 | 5 |
| A-206 | B-1 | C-2 | 80 | 15 | 5 |
| A-207 | B-1 | C-2 | 80 | 15 | 5 |
| A-208 | B-1 | C-2 | 80 | 15 | 5 |
| A-209 | B-1 | C-2 | 80 | 15 | 5 |
| A-210 | B-1 | C-2 | 80 | 15 | 5 |
| A-211 | B-1 | C-2 | 80 | 15 | 5 |
| A-212 | B-1 | C-2 | 80 | 15 | 5 |
| A-213 | B-1 | C-2 | 80 | 15 | 5 |
| A-220 | B-1 | C-2 | 80 | 15 | 5 |
| A-221 | B-1 | C-2 | 80 | 15 | 5 |
| A-222 | B-1 | C-2 | 80 | 15 | 5 |
| A-223 | B-1 | C-2 | 80 | 15 | 5 |
| A-224 | B-1 | C-2 | 80 | 15 | 5 |
| A-225 | B-1 | C-2 | 80 | 15 | 5 |
| A-226 | B-1 | C-2 | 80 | 15 | 5 |
| A-227 | B-1 | C-2 | 80 | 15 | 5 |
| A-228 | B-1 | C-2 | 80 | 15 | 5 |
| A-229 | B-1 | C-2 | 80 | 15 | 5 |
| A-230 | B-1 | C-2 | 80 | 15 | 5 |
| A-231 | B-1 | C-2 | 80 | 15 | 5 |
| A-232 | B-1 | C-2 | 80 | 15 | 5 |
| A-233 | B-1 | C-2 | 80 | 15 | 5 |
| A-234 | B-1 | C-2 | 80 | 15 | 5 |
| A-235 | B-1 | C-2 | 80 | 15 | 5 |
| A-236 | B-1 | C-2 | 80 | 15 | 5 |
| A-237 | B-1 | C-2 | 80 | 15 | 5 |
| A-238 | B-1 | C-2 | 80 | 15 | 5 |
| A-239 | B-1 | C-2 | 80 | 15 | 5 |
| A-240 | B-1 | C-2 | 80 | 15 | 5 |

TABLE 9

| | | | Ratio of | | |
|---|---|---|---|---|---|
| | | | units | | |
| | | | (mol %) | | |
| A | B | C | A | B | C |
| A-1 | B-1 | C-3 | 80 | 15 | 5 |
| A-2 | B-1 | C-3 | 80 | 15 | 5 |
| A-3 | B-1 | C-3 | 80 | 15 | 5 |
| A-4 | B-1 | C-3 | 80 | 15 | 5 |
| A-5 | B-1 | C-3 | 80 | 15 | 5 |
| A-6 | B-1 | C-3 | 80 | 15 | 5 |
| A-7 | B-1 | C-3 | 80 | 15 | 5 |
| A-8 | B-1 | C-3 | 80 | 15 | 5 |
| A-9 | B-1 | C-3 | 80 | 15 | 5 |
| A-10 | B-1 | C-3 | 80 | 15 | 5 |
| A-11 | B-1 | C-3 | 80 | 15 | 5 |
| A-12 | B-1 | C-3 | 80 | 15 | 5 |
| A-13 | B-1 | C-3 | 80 | 15 | 5 |
| A-14 | B-1 | C-3 | 80 | 15 | 5 |
| A-15 | B-1 | C-3 | 80 | 15 | 5 |
| A-16 | B-1 | C-3 | 80 | 15 | 5 |
| A-17 | B-1 | C-3 | 80 | 15 | 5 |
| A-18 | B-1 | C-3 | 80 | 15 | 5 |
| A-19 | B-1 | C-3 | 80 | 15 | 5 |
| A-20 | B-1 | C-3 | 80 | 15 | 5 |
| A-21 | B-1 | C-3 | 80 | 15 | 5 |
| A-22 | B-1 | C-3 | 80 | 15 | 5 |
| A-23 | B-1 | C-3 | 80 | 15 | 5 |
| A-24 | B-1 | C-3 | 80 | 15 | 5 |
| A-25 | B-1 | C-3 | 80 | 15 | 5 |
| A-26 | B-1 | C-3 | 80 | 15 | 5 |
| A-28 | B-1 | C-3 | 80 | 15 | 5 |
| A-29 | B-1 | C-3 | 80 | 15 | 5 |
| A-30 | B-1 | C-3 | 80 | 15 | 5 |
| A-31 | B-1 | C-3 | 80 | 15 | 5 |
| A-32 | B-1 | C-3 | 80 | 15 | 5 |
| A-33 | B-1 | C-3 | 80 | 15 | 5 |
| A-34 | B-1 | C-3 | 80 | 15 | 5 |
| A-35 | B-1 | C-3 | 80 | 15 | 5 |
| A-36 | B-1 | C-3 | 80 | 15 | 5 |
| A-37 | B-1 | C-3 | 80 | 15 | 5 |
| A-38 | B-1 | C-3 | 80 | 15 | 5 |
| A-39 | B-1 | C-3 | 80 | 15 | 5 |
| A-40 | B-1 | C-3 | 80 | 15 | 5 |
| A-41 | B-1 | C-3 | 80 | 15 | 5 |
| A-42 | B-1 | C-3 | 80 | 15 | 5 |
| A-43 | B-1 | C-3 | 80 | 15 | 5 |
| A-44 | B-1 | C-3 | 80 | 15 | 5 |
| A-45 | B-1 | C-3 | 80 | 15 | 5 |
| A-46 | B-1 | C-3 | 80 | 15 | 5 |
| A-47 | B-1 | C-3 | 80 | 15 | 5 |
| A-49 | B-1 | C-3 | 80 | 15 | 5 |
| A-50 | B-1 | C-3 | 80 | 15 | 5 |
| A-51 | B-1 | C-3 | 80 | 15 | 5 |
| A-52 | B-1 | C-3 | 80 | 15 | 5 |
| A-53 | B-1 | C-3 | 80 | 15 | 5 |
| A-54 | B-1 | C-3 | 80 | 15 | 5 |
| A-55 | B-1 | C-3 | 80 | 15 | 5 |
| A-56 | B-1 | C-3 | 80 | 15 | 5 |
| A-57 | B-1 | C-3 | 80 | 15 | 5 |
| A-58 | B-1 | C-3 | 80 | 15 | 5 |
| A-59 | B-1 | C-3 | 80 | 15 | 5 |
| A-60 | B-1 | C-3 | 80 | 15 | 5 |

TABLE 10

| | | | Ratio of | | |
|---|---|---|---|---|---|
| | | | units | | |
| | | | (mol %) | | |
| A | B | C | A | B | C |
| A-61 | B-1 | C-3 | 80 | 15 | 5 |
| A-62 | B-1 | C-3 | 80 | 15 | 5 |
| A-63 | B-1 | C-3 | 80 | 15 | 5 |
| A-64 | B-1 | C-3 | 80 | 15 | 5 |
| A-65 | B-1 | C-3 | 80 | 15 | 5 |
| A-66 | B-1 | C-3 | 80 | 15 | 5 |
| A-67 | B-1 | C-3 | 80 | 15 | 5 |
| A-68 | B-1 | C-3 | 80 | 15 | 5 |
| A-69 | B-1 | C-3 | 80 | 15 | 5 |
| A-70 | B-1 | C-3 | 80 | 15 | 5 |
| A-71 | B-1 | C-3 | 80 | 15 | 5 |
| A-72 | B-1 | C-3 | 80 | 15 | 5 |
| A-73 | B-1 | C-3 | 80 | 15 | 5 |
| A-74 | B-1 | C-3 | 80 | 15 | 5 |
| A-75 | B-1 | C-3 | 80 | 15 | 5 |
| A-76 | B-1 | C-3 | 80 | 15 | 5 |
| A-77 | B-1 | C-3 | 80 | 15 | 5 |
| A-78 | B-1 | C-3 | 80 | 15 | 5 |
| A-79 | B-1 | C-3 | 80 | 15 | 5 |
| A-80 | B-1 | C-3 | 80 | 15 | 5 |
| A-81 | B-1 | C-3 | 80 | 15 | 5 |
| A-82 | B-1 | C-3 | 80 | 15 | 5 |
| A-83 | B-1 | C-3 | 80 | 15 | 5 |
| A-84 | B-1 | C-3 | 80 | 15 | 5 |
| A-85 | B-1 | C-3 | 80 | 15 | 5 |
| A-86 | B-1 | C-3 | 80 | 15 | 5 |
| A-87 | B-1 | C-3 | 80 | 15 | 5 |
| A-88 | B-1 | C-3 | 80 | 15 | 5 |
| A-89 | B-1 | C-3 | 80 | 15 | 5 |
| A-90 | B-1 | C-3 | 80 | 15 | 5 |
| A-91 | B-1 | C-3 | 80 | 15 | 5 |
| A-92 | B-1 | C-3 | 80 | 15 | 5 |
| A-93 | B-1 | C-3 | 80 | 15 | 5 |
| A-94 | B-1 | C-3 | 80 | 15 | 5 |
| A-95 | B-1 | C-3 | 80 | 15 | 5 |
| A-96 | B-1 | C-3 | 80 | 15 | 5 |
| A-100 | B-1 | C-3 | 80 | 15 | 5 |
| A-101 | B-1 | C-3 | 80 | 15 | 5 |
| A-102 | B-1 | C-3 | 80 | 15 | 5 |
| A-103 | B-1 | C-3 | 80 | 15 | 5 |
| A-104 | B-1 | C-3 | 80 | 15 | 5 |
| A-105 | B-1 | C-3 | 80 | 15 | 5 |
| A-106 | B-1 | C-3 | 80 | 15 | 5 |
| A-107 | B-1 | C-3 | 80 | 15 | 5 |
| A-108 | B-1 | C-3 | 80 | 15 | 5 |
| A-109 | B-1 | C-3 | 80 | 15 | 5 |
| A-110 | B-1 | C-3 | 80 | 15 | 5 |
| A-111 | B-1 | C-3 | 80 | 15 | 5 |
| A-112 | B-1 | C-3 | 80 | 15 | 5 |
| A-113 | B-1 | C-3 | 80 | 15 | 5 |
| A-114 | B-1 | C-3 | 80 | 15 | 5 |
| A-115 | B-1 | C-3 | 80 | 15 | 5 |
| A-116 | B-1 | C-3 | 80 | 15 | 5 |
| A-117 | B-1 | C-3 | 80 | 15 | 5 |
| A-118 | B-1 | C-3 | 80 | 15 | 5 |
| A-119 | B-1 | C-3 | 80 | 15 | 5 |
| A-120 | B-1 | C-3 | 80 | 15 | 5 |
| A-121 | B-1 | C-3 | 80 | 15 | 5 |
| A-122 | B-1 | C-3 | 80 | 15 | 5 |
| A-123 | B-1 | C-3 | 80 | 15 | 5 |

TABLE 11

| | | | Ratio of | | |
|---|---|---|---|---|---|
| | | | units | | |
| | | | (mol %) | | |
| A | B | C | A | B | C |
| A-124 | B-1 | C-3 | 80 | 15 | 5 |
| A-125 | B-1 | C-3 | 80 | 15 | 5 |
| A-126 | B-1 | C-3 | 80 | 15 | 5 |
| A-127 | B-1 | C-3 | 80 | 15 | 5 |
| A-128 | B-1 | C-3 | 80 | 15 | 5 |
| A-129 | B-1 | C-3 | 80 | 15 | 5 |
| A-130 | B-1 | C-3 | 80 | 15 | 5 |
| A-131 | B-1 | C-3 | 80 | 15 | 5 |
| A-132 | B-1 | C-3 | 80 | 15 | 5 |
| A-133 | B-1 | C-3 | 80 | 15 | 5 |
| A-134 | B-1 | C-3 | 80 | 15 | 5 |
| A-135 | B-1 | C-3 | 80 | 15 | 5 |
| A-136 | B-1 | C-3 | 80 | 15 | 5 |
| A-137 | B-1 | C-3 | 80 | 15 | 5 |
| A-138 | B-1 | C-3 | 80 | 15 | 5 |
| A-139 | B-1 | C-3 | 80 | 15 | 5 |
| A-140 | B-1 | C-3 | 80 | 15 | 5 |
| A-141 | B-1 | C-3 | 80 | 15 | 5 |
| A-142 | B-1 | C-3 | 80 | 15 | 5 |
| A-143 | B-1 | C-3 | 80 | 15 | 5 |
| A-144 | B-1 | C-3 | 80 | 15 | 5 |
| A-145 | B-1 | C-3 | 80 | 15 | 5 |
| A-146 | B-1 | C-3 | 80 | 15 | 5 |
| A-147 | B-1 | C-3 | 80 | 15 | 5 |
| A-160 | B-1 | C-3 | 80 | 15 | 5 |
| A-161 | B-1 | C-3 | 80 | 15 | 5 |
| A-162 | B-1 | C-3 | 80 | 15 | 5 |
| A-163 | B-1 | C-3 | 80 | 15 | 5 |
| A-164 | B-1 | C-3 | 80 | 15 | 5 |
| A-165 | B-1 | C-3 | 80 | 15 | 5 |
| A-166 | B-1 | C-3 | 80 | 15 | 5 |
| A-167 | B-1 | C-3 | 80 | 15 | 5 |
| A-168 | B-1 | C-3 | 80 | 15 | 5 |
| A-169 | B-1 | C-3 | 80 | 15 | 5 |
| A-170 | B-1 | C-3 | 80 | 15 | 5 |
| A-171 | B-1 | C-3 | 80 | 15 | 5 |
| A-172 | B-1 | C-3 | 80 | 15 | 5 |
| A-173 | B-1 | C-3 | 80 | 15 | 5 |
| A-174 | B-1 | C-3 | 80 | 15 | 5 |
| A-175 | B-1 | C-3 | 80 | 15 | 5 |
| A-176 | B-1 | C-3 | 80 | 15 | 5 |
| A-177 | B-1 | C-3 | 80 | 15 | 5 |
| A-178 | B-1 | C-3 | 80 | 15 | 5 |
| A-179 | B-1 | C-3 | 80 | 15 | 5 |
| A-180 | B-1 | C-3 | 80 | 15 | 5 |
| A-181 | B-1 | C-3 | 80 | 15 | 5 |
| A-182 | B-1 | C-3 | 80 | 15 | 5 |
| A-183 | B-1 | C-3 | 80 | 15 | 5 |
| A-190 | B-1 | C-3 | 80 | 15 | 5 |
| A-191 | B-1 | C-3 | 80 | 15 | 5 |
| A-192 | B-1 | C-3 | 80 | 15 | 5 |
| A-193 | B-1 | C-3 | 80 | 15 | 5 |
| A-194 | B-1 | C-3 | 80 | 15 | 5 |
| A-195 | B-1 | C-3 | 80 | 15 | 5 |
| A-196 | B-1 | C-3 | 80 | 15 | 5 |
| A-197 | B-1 | C-3 | 80 | 15 | 5 |
| A-198 | B-1 | C-3 | 80 | 15 | 5 |
| A-199 | B-1 | C-3 | 80 | 15 | 5 |

TABLE 12

| | | | Ratio of | | |
|---|---|---|---|---|---|
| | | | units | | |
| | | | (mol %) | | |
| A | B | C | A | B | C |
| A-200 | B-1 | C-3 | 80 | 15 | 5 |
| A-201 | B-1 | C-3 | 80 | 15 | 5 |
| A-202 | B-1 | C-3 | 80 | 15 | 5 |
| A-203 | B-1 | C-3 | 80 | 15 | 5 |
| A-204 | B-1 | C-3 | 80 | 15 | 5 |
| A-205 | B-1 | C-3 | 80 | 15 | 5 |
| A-206 | B-1 | C-3 | 80 | 15 | 5 |
| A-207 | B-1 | C-3 | 80 | 15 | 5 |
| A-208 | B-1 | C-3 | 80 | 15 | 5 |
| A-209 | B-1 | C-3 | 80 | 15 | 5 |
| A-210 | B-1 | C-3 | 80 | 15 | 5 |
| A-211 | B-1 | C-3 | 80 | 15 | 5 |
| A-212 | B-1 | C-3 | 80 | 15 | 5 |
| A-213 | B-1 | C-3 | 80 | 15 | 5 |
| A-220 | B-1 | C-3 | 80 | 15 | 5 |
| A-221 | B-1 | C-3 | 80 | 15 | 5 |
| A-222 | B-1 | C-3 | 80 | 15 | 5 |
| A-223 | B-1 | C-3 | 80 | 15 | 5 |
| A-224 | B-1 | C-3 | 80 | 15 | 5 |
| A-225 | B-1 | C-3 | 80 | 15 | 5 |
| A-226 | B-1 | C-3 | 80 | 15 | 5 |
| A-227 | B-1 | C-3 | 80 | 15 | 5 |
| A-228 | B-1 | C-3 | 80 | 15 | 5 |
| A-229 | B-1 | C-3 | 80 | 15 | 5 |
| A-230 | B-1 | C-3 | 80 | 15 | 5 |
| A-231 | B-1 | C-3 | 80 | 15 | 5 |
| A-232 | B-1 | C-3 | 80 | 15 | 5 |
| A-233 | B-1 | C-3 | 80 | 15 | 5 |
| A-234 | B-1 | C-3 | 80 | 15 | 5 |
| A-235 | B-1 | C-3 | 80 | 15 | 5 |
| A-236 | B-1 | C-3 | 80 | 15 | 5 |
| A-237 | B-1 | C-3 | 80 | 15 | 5 |
| A-238 | B-1 | C-3 | 80 | 15 | 5 |
| A-239 | B-1 | C-3 | 80 | 15 | 5 |
| A-240 | B-1 | C-3 | 80 | 15 | 5 |

TABLE 13

| | | | Ratio of | | |
|---|---|---|---|---|---|
| | | | units | | |
| | | | (mol %) | | |
| A | B | C | A | B | C |
| A-1 | B-1 | C-4 | 80 | 15 | 5 |
| A-2 | B-1 | C-4 | 80 | 15 | 5 |
| A-3 | B-1 | C-4 | 80 | 15 | 5 |
| A-4 | B-1 | C-4 | 80 | 15 | 5 |
| A-5 | B-1 | C-4 | 80 | 15 | 5 |
| A-6 | B-1 | C-4 | 80 | 15 | 5 |
| A-7 | B-1 | C-4 | 80 | 15 | 5 |
| A-8 | B-1 | C-4 | 80 | 15 | 5 |
| A-9 | B-1 | C-4 | 80 | 15 | 5 |
| A-10 | B-1 | C-4 | 80 | 15 | 5 |
| A-11 | B-1 | C-4 | 80 | 15 | 5 |
| A-12 | B-1 | C-4 | 80 | 15 | 5 |
| A-13 | B-1 | C-4 | 80 | 15 | 5 |
| A-14 | B-1 | C-4 | 80 | 15 | 5 |
| A-15 | B-1 | C-4 | 80 | 15 | 5 |
| A-16 | B-1 | C-4 | 80 | 15 | 5 |
| A-17 | B-1 | C-4 | 80 | 15 | 5 |
| A-18 | B-1 | C-4 | 80 | 15 | 5 |
| A-19 | B-1 | C-4 | 80 | 15 | 5 |
| A-20 | B-1 | C-4 | 80 | 15 | 5 |
| A-21 | B-1 | C-4 | 80 | 15 | 5 |
| A-22 | B-1 | C-4 | 80 | 15 | 5 |
| A-23 | B-1 | C-4 | 80 | 15 | 5 |
| A-24 | B-1 | C-4 | 80 | 15 | 5 |
| A-25 | B-1 | C-4 | 80 | 15 | 5 |
| A-26 | B-1 | C-4 | 80 | 15 | 5 |
| A-28 | B-1 | C-4 | 80 | 15 | 5 |
| A-29 | B-1 | C-4 | 80 | 15 | 5 |
| A-30 | B-1 | C-4 | 80 | 15 | 5 |
| A-31 | B-1 | C-4 | 80 | 15 | 5 |
| A-32 | B-1 | C-4 | 80 | 15 | 5 |
| A-33 | B-1 | C-4 | 80 | 15 | 5 |
| A-34 | B-1 | C-4 | 80 | 15 | 5 |
| A-35 | B-1 | C-4 | 80 | 15 | 5 |
| A-36 | B-1 | C-4 | 80 | 15 | 5 |
| A-37 | B-1 | C-4 | 80 | 15 | 5 |
| A-38 | B-1 | C-4 | 80 | 15 | 5 |
| A-39 | B-1 | C-4 | 80 | 15 | 5 |
| A-40 | B-1 | C-4 | 80 | 15 | 5 |
| A-41 | B-1 | C-4 | 80 | 15 | 5 |
| A-42 | B-1 | C-4 | 80 | 15 | 5 |
| A-43 | B-1 | C-4 | 80 | 15 | 5 |
| A-44 | B-1 | C-4 | 80 | 15 | 5 |
| A-45 | B-1 | C-4 | 80 | 15 | 5 |
| A-46 | B-1 | C-4 | 80 | 15 | 5 |
| A-47 | B-1 | C-4 | 80 | 15 | 5 |
| A-49 | B-1 | C-4 | 80 | 15 | 5 |
| A-50 | B-1 | C-4 | 80 | 15 | 5 |
| A-51 | B-1 | C-4 | 80 | 15 | 5 |
| A-52 | B-1 | C-4 | 80 | 15 | 5 |
| A-53 | B-1 | C-4 | 80 | 15 | 5 |
| A-54 | B-1 | C-4 | 80 | 15 | 5 |
| A-55 | B-1 | C-4 | 80 | 15 | 5 |
| A-56 | B-1 | C-4 | 80 | 15 | 5 |
| A-57 | B-1 | C-4 | 80 | 15 | 5 |
| A-58 | B-1 | C-4 | 80 | 15 | 5 |
| A-59 | B-1 | C-4 | 80 | 15 | 5 |
| A-60 | B-1 | C-4 | 80 | 15 | 5 |

TABLE 14

| | | | Ratio of | | |
|---|---|---|---|---|---|
| | | | units | | |
| | | | (mol %) | | |
| A | B | C | A | B | C |
| A-61 | B-1 | C-4 | 80 | 15 | 5 |
| A-62 | B-1 | C-4 | 80 | 15 | 5 |
| A-63 | B-1 | C-4 | 80 | 15 | 5 |
| A-64 | B-1 | C-4 | 80 | 15 | 5 |
| A-65 | B-1 | C-4 | 80 | 15 | 5 |
| A-66 | B-1 | C-4 | 80 | 15 | 5 |
| A-67 | B-1 | C-4 | 80 | 15 | 5 |
| A-68 | B-1 | C-4 | 80 | 15 | 5 |
| A-69 | B-1 | C-4 | 80 | 15 | 5 |
| A-70 | B-1 | C-4 | 80 | 15 | 5 |
| A-71 | B-1 | C-4 | 80 | 15 | 5 |
| A-72 | B-1 | C-4 | 80 | 15 | 5 |
| A-73 | B-1 | C-4 | 80 | 15 | 5 |
| A-74 | B-1 | C-4 | 80 | 15 | 5 |
| A-75 | B-1 | C-4 | 80 | 15 | 5 |
| A-76 | B-1 | C-4 | 80 | 15 | 5 |
| A-77 | B-1 | C-4 | 80 | 15 | 5 |
| A-78 | B-1 | C-4 | 80 | 15 | 5 |
| A-79 | B-1 | C-4 | 80 | 15 | 5 |
| A-80 | B-1 | C-4 | 80 | 15 | 5 |
| A-81 | B-1 | C-4 | 80 | 15 | 5 |
| A-82 | B-1 | C-4 | 80 | 15 | 5 |
| A-83 | B-1 | C-4 | 80 | 15 | 5 |
| A-84 | B-1 | C-4 | 80 | 15 | 5 |
| A-85 | B-1 | C-4 | 80 | 15 | 5 |
| A-86 | B-1 | C-4 | 80 | 15 | 5 |
| A-87 | B-1 | C-4 | 80 | 15 | 5 |
| A-88 | B-1 | C-4 | 80 | 15 | 5 |
| A-89 | B-1 | C-4 | 80 | 15 | 5 |
| A-90 | B-1 | C-4 | 80 | 15 | 5 |
| A-91 | B-1 | C-4 | 80 | 15 | 5 |
| A-92 | B-1 | C-4 | 80 | 15 | 5 |
| A-93 | B-1 | C-4 | 80 | 15 | 5 |
| A-94 | B-1 | C-4 | 80 | 15 | 5 |
| A-95 | B-1 | C-4 | 80 | 15 | 5 |
| A-96 | B-1 | C-4 | 80 | 15 | 5 |
| A-100 | B-1 | C-4 | 80 | 15 | 5 |
| A-101 | B-1 | C-4 | 80 | 15 | 5 |
| A-102 | B-1 | C-4 | 80 | 15 | 5 |
| A-103 | B-1 | C-4 | 80 | 15 | 5 |
| A-104 | B-1 | C-4 | 80 | 15 | 5 |
| A-105 | B-1 | C-4 | 80 | 15 | 5 |
| A-106 | B-1 | C-4 | 80 | 15 | 5 |
| A-107 | B-1 | C-4 | 80 | 15 | 5 |
| A-108 | B-1 | C-4 | 80 | 15 | 5 |
| A-109 | B-1 | C-4 | 80 | 15 | 5 |
| A-110 | B-1 | C-4 | 80 | 15 | 5 |
| A-111 | B-1 | C-4 | 80 | 15 | 5 |
| A-112 | B-1 | C-4 | 80 | 15 | 5 |
| A-113 | B-1 | C-4 | 80 | 15 | 5 |
| A-114 | B-1 | C-4 | 80 | 15 | 5 |
| A-115 | B-1 | C-4 | 80 | 15 | 5 |
| A-116 | B-1 | C-4 | 80 | 15 | 5 |
| A-117 | B-1 | C-4 | 80 | 15 | 5 |
| A-118 | B-1 | C-4 | 80 | 15 | 5 |
| A-119 | B-1 | C-4 | 80 | 15 | 5 |
| A-120 | B-1 | C-4 | 80 | 15 | 5 |
| A-121 | B-1 | C-4 | 80 | 15 | 5 |
| A-122 | B-1 | C-4 | 80 | 15 | 5 |
| A-123 | B-1 | C-4 | 80 | 15 | 5 |

TABLE 15

| | | | Ratio of | | |
|---|---|---|---|---|---|
| | | | units | | |
| | | | (mol %) | | |
| A | B | C | A | B | C |
| A-124 | B-1 | C-4 | 80 | 15 | 5 |
| A-125 | B-1 | C-4 | 80 | 15 | 5 |
| A-126 | B-1 | C-4 | 80 | 15 | 5 |
| A-127 | B-1 | C-4 | 80 | 15 | 5 |
| A-128 | B-1 | C-4 | 80 | 15 | 5 |
| A-129 | B-1 | C-4 | 80 | 15 | 5 |
| A-130 | B-1 | C-4 | 80 | 15 | 5 |
| A-131 | B-1 | C-4 | 80 | 15 | 5 |
| A-132 | B-1 | C-4 | 80 | 15 | 5 |
| A-133 | B-1 | C-4 | 80 | 15 | 5 |
| A-134 | B-1 | C-4 | 80 | 15 | 5 |
| A-135 | B-1 | C-4 | 80 | 15 | 5 |
| A-136 | B-1 | C-4 | 80 | 15 | 5 |
| A-137 | B-1 | C-4 | 80 | 15 | 5 |
| A-138 | B-1 | C-4 | 80 | 15 | 5 |
| A-139 | B-1 | C-4 | 80 | 15 | 5 |
| A-140 | B-1 | C-4 | 80 | 15 | 5 |
| A-141 | B-1 | C-4 | 80 | 15 | 5 |
| A-142 | B-1 | C-4 | 80 | 15 | 5 |
| A-143 | B-1 | C-4 | 80 | 15 | 5 |
| A-144 | B-1 | C-4 | 80 | 15 | 5 |
| A-145 | B-1 | C-4 | 80 | 15 | 5 |
| A-146 | B-1 | C-4 | 80 | 15 | 5 |
| A-147 | B-1 | C-4 | 80 | 15 | 5 |
| A-160 | B-1 | C-4 | 80 | 15 | 5 |
| A-161 | B-1 | C-4 | 80 | 15 | 5 |
| A-162 | B-1 | C-4 | 80 | 15 | 5 |
| A-163 | B-1 | C-4 | 80 | 15 | 5 |
| A-164 | B-1 | C-4 | 80 | 15 | 5 |
| A-165 | B-1 | C-4 | 80 | 15 | 5 |
| A-166 | B-1 | C-4 | 80 | 15 | 5 |
| A-167 | B-1 | C-4 | 80 | 15 | 5 |
| A-168 | B-1 | C-4 | 80 | 15 | 5 |
| A-169 | B-1 | C-4 | 80 | 15 | 5 |
| A-170 | B-1 | C-4 | 80 | 15 | 5 |
| A-171 | B-1 | C-4 | 80 | 15 | 5 |
| A-172 | B-1 | C-4 | 80 | 15 | 5 |
| A-173 | B-1 | C-4 | 80 | 15 | 5 |
| A-174 | B-1 | C-4 | 80 | 15 | 5 |
| A-175 | B-1 | C-4 | 80 | 15 | 5 |
| A-176 | B-1 | C-4 | 80 | 15 | 5 |
| A-177 | B-1 | C-4 | 80 | 15 | 5 |
| A-178 | B-1 | C-4 | 80 | 15 | 5 |
| A-179 | B-1 | C-4 | 80 | 15 | 5 |
| A-180 | B-1 | C-4 | 80 | 15 | 5 |
| A-181 | B-1 | C-4 | 80 | 15 | 5 |
| A-182 | B-1 | C-4 | 80 | 15 | 5 |
| A-183 | B-1 | C-4 | 80 | 15 | 5 |
| A-190 | B-1 | C-4 | 80 | 15 | 5 |
| A-191 | B-1 | C-4 | 80 | 15 | 5 |
| A-192 | B-1 | C-4 | 80 | 15 | 5 |
| A-193 | B-1 | C-4 | 80 | 15 | 5 |
| A-194 | B-1 | C-4 | 80 | 15 | 5 |
| A-195 | B-1 | C-4 | 80 | 15 | 5 |
| A-196 | B-1 | C-4 | 80 | 15 | 5 |
| A-197 | B-1 | C-4 | 80 | 15 | 5 |
| A-198 | B-1 | C-4 | 80 | 15 | 5 |
| A-199 | B-1 | C-4 | 80 | 15 | 5 |

TABLE 16

| | | | Ratio of | | |
|---|---|---|---|---|---|
| | | | units | | |
| | | | (mol %) | | |
| A | B | C | A | B | C |
| A-200 | B-1 | C-4 | 80 | 15 | 5 |
| A-201 | B-1 | C-4 | 80 | 15 | 5 |
| A-202 | B-1 | C-4 | 80 | 15 | 5 |
| A-203 | B-1 | C-4 | 80 | 15 | 5 |
| A-204 | B-1 | C-4 | 80 | 15 | 5 |
| A-205 | B-1 | C-4 | 80 | 15 | 5 |
| A-206 | B-1 | C-4 | 80 | 15 | 5 |
| A-207 | B-1 | C-4 | 80 | 15 | 5 |
| A-208 | B-1 | C-4 | 80 | 15 | 5 |
| A-209 | B-1 | C-4 | 80 | 15 | 5 |
| A-210 | B-1 | C-4 | 80 | 15 | 5 |
| A-211 | B-1 | C-4 | 80 | 15 | 5 |
| A-212 | B-1 | C-4 | 80 | 15 | 5 |
| A-213 | B-1 | C-4 | 80 | 15 | 5 |
| A-220 | B-1 | C-4 | 80 | 15 | 5 |
| A-221 | B-1 | C-4 | 80 | 15 | 5 |
| A-222 | B-1 | C-4 | 80 | 15 | 5 |
| A-223 | B-1 | C-4 | 80 | 15 | 5 |
| A-224 | B-1 | C-4 | 80 | 15 | 5 |
| A-225 | B-1 | C-4 | 80 | 15 | 5 |
| A-226 | B-1 | C-4 | 80 | 15 | 5 |
| A-227 | B-1 | C-4 | 80 | 15 | 5 |
| A-228 | B-1 | C-4 | 80 | 15 | 5 |
| A-229 | B-1 | C-4 | 80 | 15 | 5 |
| A-230 | B-1 | C-4 | 80 | 15 | 5 |
| A-231 | B-1 | C-4 | 80 | 15 | 5 |
| A-232 | B-1 | C-4 | 80 | 15 | 5 |
| A-233 | B-1 | C-4 | 80 | 15 | 5 |
| A-234 | B-1 | C-4 | 80 | 15 | 5 |
| A-235 | B-1 | C-4 | 80 | 15 | 5 |
| A-236 | B-1 | C-4 | 80 | 15 | 5 |
| A-237 | B-1 | C-4 | 80 | 15 | 5 |
| A-238 | B-1 | C-4 | 80 | 15 | 5 |
| A-239 | B-1 | C-4 | 80 | 15 | 5 |
| A-240 | B-1 | C-4 | 80 | 15 | 5 |

TABLE 17

| | | | Ratio | | |
|---|---|---|---|---|---|
| | | | of units | | |
| | | | (mol %) | | |
| A | B | C | A | B | C |
| A-1 | B-2 | C-1 | 80 | 15 | 5 |
| A-2 | B-2 | C-1 | 80 | 15 | 5 |
| A-3 | B-2 | C-1 | 80 | 15 | 5 |
| A-4 | B-2 | C-1 | 80 | 15 | 5 |
| A-5 | B-2 | C-1 | 80 | 15 | 5 |
| A-6 | B-2 | C-1 | 80 | 15 | 5 |
| A-7 | B-2 | C-1 | 80 | 15 | 5 |
| A-8 | B-2 | C-1 | 80 | 15 | 5 |
| A-9 | B-2 | C-1 | 80 | 15 | 5 |
| A-10 | B-2 | C-1 | 80 | 15 | 5 |
| A-11 | B-2 | C-1 | 80 | 15 | 5 |
| A-12 | B-2 | C-1 | 80 | 15 | 5 |
| A-13 | B-2 | C-1 | 80 | 15 | 5 |
| A-14 | B-2 | C-1 | 80 | 15 | 5 |
| A-15 | B-2 | C-1 | 80 | 15 | 5 |
| A-16 | B-2 | C-1 | 80 | 15 | 5 |
| A-17 | B-2 | C-1 | 80 | 15 | 5 |
| A-18 | B-2 | C-1 | 80 | 15 | 5 |
| A-19 | B-2 | C-1 | 80 | 15 | 5 |
| A-20 | B-2 | C-1 | 80 | 15 | 5 |
| A-21 | B-2 | C-1 | 80 | 15 | 5 |
| A-22 | B-2 | C-1 | 80 | 15 | 5 |
| A-23 | B-2 | C-1 | 80 | 15 | 5 |
| A-24 | B-2 | C-1 | 80 | 15 | 5 |
| A-25 | B-2 | C-1 | 80 | 15 | 5 |
| A-26 | B-2 | C-1 | 80 | 15 | 5 |
| A-28 | B-2 | C-1 | 80 | 15 | 5 |
| A-29 | B-2 | C-1 | 80 | 15 | 5 |
| A-30 | B-2 | C-1 | 80 | 15 | 5 |
| A-31 | B-2 | C-1 | 80 | 15 | 5 |
| A-32 | B-2 | C-1 | 80 | 15 | 5 |
| A-33 | B-2 | C-1 | 80 | 15 | 5 |
| A-34 | B-2 | C-1 | 80 | 15 | 5 |
| A-35 | B-2 | C-1 | 80 | 15 | 5 |
| A-36 | B-2 | C-1 | 80 | 15 | 5 |
| A-37 | B-2 | C-1 | 80 | 15 | 5 |
| A-38 | B-2 | C-1 | 80 | 15 | 5 |
| A-39 | B-2 | C-1 | 80 | 15 | 5 |
| A-40 | B-2 | C-1 | 80 | 15 | 5 |
| A-41 | B-2 | C-1 | 80 | 15 | 5 |
| A-42 | B-2 | C-1 | 80 | 15 | 5 |
| A-43 | B-2 | C-1 | 80 | 15 | 5 |
| A-44 | B-2 | C-1 | 80 | 15 | 5 |
| A-45 | B-2 | C-1 | 80 | 15 | 5 |
| A-46 | B-2 | C-1 | 80 | 15 | 5 |
| A-47 | B-2 | C-1 | 80 | 15 | 5 |
| A-49 | B-2 | C-1 | 80 | 15 | 5 |
| A-50 | B-2 | C-1 | 80 | 15 | 5 |
| A-51 | B-2 | C-1 | 80 | 15 | 5 |
| A-52 | B-2 | C-1 | 80 | 15 | 5 |
| A-53 | B-2 | C-1 | 80 | 15 | 5 |
| A-54 | B-2 | C-1 | 80 | 15 | 5 |
| A-55 | B-2 | C-1 | 80 | 15 | 5 |
| A-56 | B-2 | C-1 | 80 | 15 | 5 |
| A-57 | B-2 | C-1 | 80 | 15 | 5 |
| A-58 | B-2 | C-1 | 80 | 15 | 5 |
| A-59 | B-2 | C-1 | 80 | 15 | 5 |
| A-60 | B-2 | C-1 | 80 | 15 | 5 |

TABLE 18

| | | | Ratio | | |
|---|---|---|---|---|---|
| | | | of units | | |
| | | | (mol %) | | |
| A | B | C | A | B | C |
| A-61 | B-2 | C-1 | 80 | 15 | 5 |
| A-62 | B-2 | C-1 | 80 | 15 | 5 |
| A-63 | B-2 | C-1 | 80 | 15 | 5 |
| A-64 | B-2 | C-1 | 80 | 15 | 5 |
| A-65 | B-2 | C-1 | 80 | 15 | 5 |
| A-66 | B-2 | C-1 | 80 | 15 | 5 |
| A-67 | B-2 | C-1 | 80 | 15 | 5 |
| A-68 | B-2 | C-1 | 80 | 15 | 5 |
| A-69 | B-2 | C-1 | 80 | 15 | 5 |
| A-70 | B-2 | C-1 | 80 | 15 | 5 |
| A-71 | B-2 | C-1 | 80 | 15 | 5 |
| A-72 | B-2 | C-1 | 80 | 15 | 5 |
| A-73 | B-2 | C-1 | 80 | 15 | 5 |
| A-74 | B-2 | C-1 | 80 | 15 | 5 |
| A-75 | B-2 | C-1 | 80 | 15 | 5 |
| A-76 | B-2 | C-1 | 80 | 15 | 5 |
| A-77 | B-2 | C-1 | 80 | 15 | 5 |
| A-78 | B-2 | C-1 | 80 | 15 | 5 |
| A-79 | B-2 | C-1 | 80 | 15 | 5 |
| A-80 | B-2 | C-1 | 80 | 15 | 5 |
| A-81 | B-2 | C-1 | 80 | 15 | 5 |
| A-82 | B-2 | C-1 | 80 | 15 | 5 |
| A-83 | B-2 | C-1 | 80 | 15 | 5 |
| A-84 | B-2 | C-1 | 80 | 15 | 5 |
| A-85 | B-2 | C-1 | 80 | 15 | 5 |
| A-86 | B-2 | C-1 | 80 | 15 | 5 |
| A-87 | B-2 | C-1 | 80 | 15 | 5 |
| A-88 | B-2 | C-1 | 80 | 15 | 5 |
| A-89 | B-2 | C-1 | 80 | 15 | 5 |
| A-90 | B-2 | C-1 | 80 | 15 | 5 |
| A-91 | B-2 | C-1 | 80 | 15 | 5 |
| A-92 | B-2 | C-1 | 80 | 15 | 5 |
| A-93 | B-2 | C-1 | 80 | 15 | 5 |
| A-94 | B-2 | C-1 | 80 | 15 | 5 |
| A-95 | B-2 | C-1 | 80 | 15 | 5 |
| A-96 | B-2 | C-1 | 80 | 15 | 5 |
| A-100 | B-2 | C-1 | 80 | 15 | 5 |
| A-101 | B-2 | C-1 | 80 | 15 | 5 |
| A-102 | B-2 | C-1 | 80 | 15 | 5 |
| A-103 | B-2 | C-1 | 80 | 15 | 5 |
| A-104 | B-2 | C-1 | 80 | 15 | 5 |
| A-105 | B-2 | C-1 | 80 | 15 | 5 |
| A-106 | B-2 | C-1 | 80 | 15 | 5 |
| A-107 | B-2 | C-1 | 80 | 15 | 5 |
| A-108 | B-2 | C-1 | 80 | 15 | 5 |
| A-109 | B-2 | C-1 | 80 | 15 | 5 |
| A-110 | B-2 | C-1 | 80 | 15 | 5 |
| A-111 | B-2 | C-1 | 80 | 15 | 5 |
| A-112 | B-2 | C-1 | 80 | 15 | 5 |
| A-113 | B-2 | C-1 | 80 | 15 | 5 |
| A-114 | B-2 | C-1 | 80 | 15 | 5 |
| A-115 | B-2 | C-1 | 80 | 15 | 5 |
| A-116 | B-2 | C-1 | 80 | 15 | 5 |
| A-117 | B-2 | C-1 | 80 | 15 | 5 |
| A-118 | B-2 | C-1 | 80 | 15 | 5 |
| A-119 | B-2 | C-1 | 80 | 15 | 5 |
| A-120 | B-2 | C-1 | 80 | 15 | 5 |
| A-121 | B-2 | C-1 | 80 | 15 | 5 |
| A-122 | B-2 | C-1 | 80 | 15 | 5 |
| A-123 | B-2 | C-1 | 80 | 15 | 5 |

TABLE 19

| | | | Ratio | | |
|---|---|---|---|---|---|
| | | | of units | | |
| | | | (mol %) | | |
| A | B | C | A | B | C |
| A-124 | B-2 | C-1 | 80 | 15 | 5 |
| A-125 | B-2 | C-1 | 80 | 15 | 5 |
| A-126 | B-2 | C-1 | 80 | 15 | 5 |
| A-127 | B-2 | C-1 | 80 | 15 | 5 |
| A-128 | B-2 | C-1 | 80 | 15 | 5 |
| A-129 | B-2 | C-1 | 80 | 15 | 5 |
| A-130 | B-2 | C-1 | 80 | 15 | 5 |
| A-131 | B-2 | C-1 | 80 | 15 | 5 |
| A-132 | B-2 | C-1 | 80 | 15 | 5 |
| A-133 | B-2 | C-1 | 80 | 15 | 5 |
| A-134 | B-2 | C-1 | 80 | 15 | 5 |
| A-135 | B-2 | C-1 | 80 | 15 | 5 |
| A-136 | B-2 | C-1 | 80 | 15 | 5 |
| A-137 | B-2 | C-1 | 80 | 15 | 5 |
| A-138 | B-2 | C-1 | 80 | 15 | 5 |
| A-139 | B-2 | C-1 | 80 | 15 | 5 |
| A-140 | B-2 | C-1 | 80 | 15 | 5 |
| A-141 | B-2 | C-1 | 80 | 15 | 5 |
| A-142 | B-2 | C-1 | 80 | 15 | 5 |
| A-143 | B-2 | C-1 | 80 | 15 | 5 |
| A-144 | B-2 | C-1 | 80 | 15 | 5 |
| A-145 | B-2 | C-1 | 80 | 15 | 5 |
| A-146 | B-2 | C-1 | 80 | 15 | 5 |
| A-147 | B-2 | C-1 | 80 | 15 | 5 |
| A-160 | B-2 | C-1 | 80 | 15 | 5 |
| A-161 | B-2 | C-1 | 80 | 15 | 5 |
| A-162 | B-2 | C-1 | 80 | 15 | 5 |
| A-163 | B-2 | C-1 | 80 | 15 | 5 |
| A-164 | B-2 | C-1 | 80 | 15 | 5 |
| A-165 | B-2 | C-1 | 80 | 15 | 5 |
| A-166 | B-2 | C-1 | 80 | 15 | 5 |
| A-167 | B-2 | C-1 | 80 | 15 | 5 |
| A-168 | B-2 | C-1 | 80 | 15 | 5 |
| A-169 | B-2 | C-1 | 80 | 15 | 5 |
| A-170 | B-2 | C-1 | 80 | 15 | 5 |
| A-171 | B-2 | C-1 | 80 | 15 | 5 |
| A-172 | B-2 | C-1 | 80 | 15 | 5 |
| A-173 | B-2 | C-1 | 80 | 15 | 5 |
| A-174 | B-2 | C-1 | 80 | 15 | 5 |
| A-175 | B-2 | C-1 | 80 | 15 | 5 |
| A-176 | B-2 | C-1 | 80 | 15 | 5 |
| A-177 | B-2 | C-1 | 80 | 15 | 5 |
| A-178 | B-2 | C-1 | 80 | 15 | 5 |
| A-179 | B-2 | C-1 | 80 | 15 | 5 |
| A-180 | B-2 | C-1 | 80 | 15 | 5 |
| A-181 | B-2 | C-1 | 80 | 15 | 5 |
| A-182 | B-2 | C-1 | 80 | 15 | 5 |
| A-183 | B-2 | C-1 | 80 | 15 | 5 |
| A-190 | B-2 | C-1 | 80 | 15 | 5 |
| A-191 | B-2 | C-1 | 80 | 15 | 5 |
| A-192 | B-2 | C-1 | 80 | 15 | 5 |
| A-193 | B-2 | C-1 | 80 | 15 | 5 |
| A-194 | B-2 | C-1 | 80 | 15 | 5 |
| A-195 | B-2 | C-1 | 80 | 15 | 5 |
| A-196 | B-2 | C-1 | 80 | 15 | 5 |
| A-197 | B-2 | C-1 | 80 | 15 | 5 |
| A-198 | B-2 | C-1 | 80 | 15 | 5 |
| A-199 | B-2 | C-1 | 80 | 15 | 5 |

TABLE 20

| | | | Ratio of units (mol %) | | |
|---|---|---|---|---|---|
| A | B | C | A | B | C |
| A-200 | B-2 | C-1 | 80 | 15 | 5 |
| A-201 | B-2 | C-1 | 80 | 15 | 5 |
| A-202 | B-2 | C-1 | 80 | 15 | 5 |
| A-203 | B-2 | C-1 | 80 | 15 | 5 |
| A-204 | B-2 | C-1 | 80 | 15 | 5 |
| A-205 | B-2 | C-1 | 80 | 15 | 5 |
| A-206 | B-2 | C-1 | 80 | 15 | 5 |
| A-207 | B-2 | C-1 | 80 | 15 | 5 |
| A-208 | B-2 | C-1 | 80 | 15 | 5 |
| A-209 | B-2 | C-1 | 80 | 15 | 5 |
| A-210 | B-2 | C-1 | 80 | 15 | 5 |
| A-211 | B-2 | C-1 | 80 | 15 | 5 |
| A-212 | B-2 | C-1 | 80 | 15 | 5 |
| A-213 | B-2 | C-1 | 80 | 15 | 5 |
| A-220 | B-2 | C-1 | 80 | 15 | 5 |
| A-221 | B-2 | C-1 | 80 | 15 | 5 |
| A-222 | B-2 | C-1 | 80 | 15 | 5 |
| A-223 | B-2 | C-1 | 80 | 15 | 5 |
| A-224 | B-2 | C-1 | 80 | 15 | 5 |
| A-225 | B-2 | C-1 | 80 | 15 | 5 |
| A-226 | B-2 | C-1 | 80 | 15 | 5 |
| A-227 | B-2 | C-1 | 80 | 15 | 5 |
| A-228 | B-2 | C-1 | 80 | 15 | 5 |
| A-229 | B-2 | C-1 | 80 | 15 | 5 |
| A-230 | B-2 | C-1 | 80 | 15 | 5 |
| A-231 | B-2 | C-1 | 80 | 15 | 5 |
| A-232 | B-2 | C-1 | 80 | 15 | 5 |
| A-233 | B-2 | C-1 | 80 | 15 | 5 |
| A-234 | B-2 | C-1 | 80 | 15 | 5 |
| A-235 | B-2 | C-1 | 80 | 15 | 5 |
| A-236 | B-2 | C-1 | 80 | 15 | 5 |
| A-237 | B-2 | C-1 | 80 | 15 | 5 |
| A-238 | B-2 | C-1 | 80 | 15 | 5 |
| A-239 | B-2 | C-1 | 80 | 15 | 5 |
| A-240 | B-2 | C-1 | 80 | 15 | 5 |

TABLE 21

| | | | Ratio of units (mol %) | | |
|---|---|---|---|---|---|
| A | B | C | A | B | C |
| A-1 | B-2 | C-2 | 80 | 15 | 5 |
| A-2 | B-2 | C-2 | 80 | 15 | 5 |
| A-3 | B-2 | C-2 | 80 | 15 | 5 |
| A-4 | B-2 | C-2 | 80 | 15 | 5 |
| A-5 | B-2 | C-2 | 80 | 15 | 5 |
| A-6 | B-2 | C-2 | 80 | 15 | 5 |
| A-7 | B-2 | C-2 | 80 | 15 | 5 |
| A-8 | B-2 | C-2 | 80 | 15 | 5 |
| A-9 | B-2 | C-2 | 80 | 15 | 5 |
| A-10 | B-2 | C-2 | 80 | 15 | 5 |
| A-11 | B-2 | C-2 | 80 | 15 | 5 |
| A-12 | B-2 | C-2 | 80 | 15 | 5 |
| A-13 | B-2 | C-2 | 80 | 15 | 5 |
| A-14 | B-2 | C-2 | 80 | 15 | 5 |
| A-15 | B-2 | C-2 | 80 | 15 | 5 |
| A-16 | B-2 | C-2 | 80 | 15 | 5 |
| A-17 | B-2 | C-2 | 80 | 15 | 5 |
| A-18 | B-2 | C-2 | 80 | 15 | 5 |
| A-19 | B-2 | C-2 | 80 | 15 | 5 |
| A-20 | B-2 | C-2 | 80 | 15 | 5 |
| A-21 | B-2 | C-2 | 80 | 15 | 5 |
| A-22 | B-2 | C-2 | 80 | 15 | 5 |
| A-23 | B-2 | C-2 | 80 | 15 | 5 |
| A-24 | B-2 | C-2 | 80 | 15 | 5 |
| A-25 | B-2 | C-2 | 80 | 15 | 5 |
| A-26 | B-2 | C-2 | 80 | 15 | 5 |
| A-28 | B-2 | C-2 | 80 | 15 | 5 |
| A-29 | B-2 | C-2 | 80 | 15 | 5 |
| A-30 | B-2 | C-2 | 80 | 15 | 5 |
| A-31 | B-2 | C-2 | 80 | 15 | 5 |
| A-32 | B-2 | C-2 | 80 | 15 | 5 |
| A-33 | B-2 | C-2 | 80 | 15 | 5 |
| A-34 | B-2 | C-2 | 80 | 15 | 5 |
| A-35 | B-2 | C-2 | 80 | 15 | 5 |
| A-36 | B-2 | C-2 | 80 | 15 | 5 |
| A-37 | B-2 | C-2 | 80 | 15 | 5 |
| A-38 | B-2 | C-2 | 80 | 15 | 5 |
| A-39 | B-2 | C-2 | 80 | 15 | 5 |
| A-40 | B-2 | C-2 | 80 | 15 | 5 |
| A-41 | B-2 | C-2 | 80 | 15 | 5 |
| A-42 | B-2 | C-2 | 80 | 15 | 5 |
| A-43 | B-2 | C-2 | 80 | 15 | 5 |
| A-44 | B-2 | C-2 | 80 | 15 | 5 |
| A-45 | B-2 | C-2 | 80 | 15 | 5 |
| A-46 | B-2 | C-2 | 80 | 15 | 5 |
| A-47 | B-2 | C-2 | 80 | 15 | 5 |
| A-49 | B-2 | C-2 | 80 | 15 | 5 |
| A-50 | B-2 | C-2 | 80 | 15 | 5 |
| A-51 | B-2 | C-2 | 80 | 15 | 5 |
| A-52 | B-2 | C-2 | 80 | 15 | 5 |
| A-53 | B-2 | C-2 | 80 | 15 | 5 |
| A-54 | B-2 | C-2 | 80 | 15 | 5 |
| A-55 | B-2 | C-2 | 80 | 15 | 5 |
| A-56 | B-2 | C-2 | 80 | 15 | 5 |
| A-57 | B-2 | C-2 | 80 | 15 | 5 |
| A-58 | B-2 | C-2 | 80 | 15 | 5 |
| A-59 | B-2 | C-2 | 80 | 15 | 5 |
| A-60 | B-2 | C-2 | 80 | 15 | 5 |

TABLE 22

| | | | Ratio of units (mol %) | | |
|---|---|---|---|---|---|
| A | B | C | A | B | C |
| A-61 | B-2 | C-2 | 80 | 15 | 5 |
| A-62 | B-2 | C-2 | 80 | 15 | 5 |
| A-63 | B-2 | C-2 | 80 | 15 | 5 |
| A-64 | B-2 | C-2 | 80 | 15 | 5 |
| A-65 | B-2 | C-2 | 80 | 15 | 5 |
| A-66 | B-2 | C-2 | 80 | 15 | 5 |
| A-67 | B-2 | C-2 | 80 | 15 | 5 |
| A-68 | B-2 | C-2 | 80 | 15 | 5 |
| A-69 | B-2 | C-2 | 80 | 15 | 5 |
| A-70 | B-2 | C-2 | 80 | 15 | 5 |
| A-71 | B-2 | C-2 | 80 | 15 | 5 |
| A-72 | B-2 | C-2 | 80 | 15 | 5 |
| A-73 | B-2 | C-2 | 80 | 15 | 5 |
| A-74 | B-2 | C-2 | 80 | 15 | 5 |
| A-75 | B-2 | C-2 | 80 | 15 | 5 |
| A-76 | B-2 | C-2 | 80 | 15 | 5 |
| A-77 | B-2 | C-2 | 80 | 15 | 5 |
| A-78 | B-2 | C-2 | 80 | 15 | 5 |
| A-79 | B-2 | C-2 | 80 | 15 | 5 |
| A-80 | B-2 | C-2 | 80 | 15 | 5 |
| A-81 | B-2 | C-2 | 80 | 15 | 5 |
| A-82 | B-2 | C-2 | 80 | 15 | 5 |
| A-83 | B-2 | C-2 | 80 | 15 | 5 |
| A-84 | B-2 | C-2 | 80 | 15 | 5 |
| A-85 | B-2 | C-2 | 80 | 15 | 5 |
| A-86 | B-2 | C-2 | 80 | 15 | 5 |
| A-87 | B-2 | C-2 | 80 | 15 | 5 |
| A-88 | B-2 | C-2 | 80 | 15 | 5 |
| A-89 | B-2 | C-2 | 80 | 15 | 5 |
| A-90 | B-2 | C-2 | 80 | 15 | 5 |
| A-91 | B-2 | C-2 | 80 | 15 | 5 |
| A-92 | B-2 | C-2 | 80 | 15 | 5 |
| A-93 | B-2 | C-2 | 80 | 15 | 5 |
| A-94 | B-2 | C-2 | 80 | 15 | 5 |
| A-95 | B-2 | C-2 | 80 | 15 | 5 |
| A-96 | B-2 | C-2 | 80 | 15 | 5 |
| A-100 | B-2 | C-2 | 80 | 15 | 5 |
| A-101 | B-2 | C-2 | 80 | 15 | 5 |
| A-102 | B-2 | C-2 | 80 | 15 | 5 |
| A-103 | B-2 | C-2 | 80 | 15 | 5 |
| A-104 | B-2 | C-2 | 80 | 15 | 5 |
| A-105 | B-2 | C-2 | 80 | 15 | 5 |
| A-106 | B-2 | C-2 | 80 | 15 | 5 |
| A-107 | B-2 | C-2 | 80 | 15 | 5 |
| A-108 | B-2 | C-2 | 80 | 15 | 5 |
| A-109 | B-2 | C-2 | 80 | 15 | 5 |
| A-110 | B-2 | C-2 | 80 | 15 | 5 |
| A-111 | B-2 | C-2 | 80 | 15 | 5 |
| A-112 | B-2 | C-2 | 80 | 15 | 5 |
| A-113 | B-2 | C-2 | 80 | 15 | 5 |
| A-114 | B-2 | C-2 | 80 | 15 | 5 |
| A-115 | B-2 | C-2 | 80 | 15 | 5 |
| A-116 | B-2 | C-2 | 80 | 15 | 5 |
| A-117 | B-2 | C-2 | 80 | 15 | 5 |
| A-118 | B-2 | C-2 | 80 | 15 | 5 |
| A-119 | B-2 | C-2 | 80 | 15 | 5 |
| A-120 | B-2 | C-2 | 80 | 15 | 5 |
| A-121 | B-2 | C-2 | 80 | 15 | 5 |
| A-122 | B-2 | C-2 | 80 | 15 | 5 |
| A-123 | B-2 | C-2 | 80 | 15 | 5 |

TABLE 23

| | | | Ratio of units (mol %) | | |
|---|---|---|---|---|---|
| A | B | C | A | B | C |
| A-124 | B-2 | C-2 | 80 | 15 | 5 |
| A-125 | B-2 | C-2 | 80 | 15 | 5 |
| A-126 | B-2 | C-2 | 80 | 15 | 5 |
| A-127 | B-2 | C-2 | 80 | 15 | 5 |
| A-128 | B-2 | C-2 | 80 | 15 | 5 |
| A-129 | B-2 | C-2 | 80 | 15 | 5 |
| A-130 | B-2 | C-2 | 80 | 15 | 5 |
| A-131 | B-2 | C-2 | 80 | 15 | 5 |
| A-132 | B-2 | C-2 | 80 | 15 | 5 |
| A-133 | B-2 | C-2 | 80 | 15 | 5 |
| A-134 | B-2 | C-2 | 80 | 15 | 5 |
| A-135 | B-2 | C-2 | 80 | 15 | 5 |
| A-136 | B-2 | C-2 | 80 | 15 | 5 |
| A-137 | B-2 | C-2 | 80 | 15 | 5 |
| A-138 | B-2 | C-2 | 80 | 15 | 5 |
| A-139 | B-2 | C-2 | 80 | 15 | 5 |
| A-140 | B-2 | C-2 | 80 | 15 | 5 |
| A-141 | B-2 | C-2 | 80 | 15 | 5 |
| A-142 | B-2 | C-2 | 80 | 15 | 5 |
| A-143 | B-2 | C-2 | 80 | 15 | 5 |
| A-144 | B-2 | C-2 | 80 | 15 | 5 |
| A-145 | B-2 | C-2 | 80 | 15 | 5 |
| A-146 | B-2 | C-2 | 80 | 15 | 5 |
| A-147 | B-2 | C-2 | 80 | 15 | 5 |
| A-160 | B-2 | C-2 | 80 | 15 | 5 |
| A-161 | B-2 | C-2 | 80 | 15 | 5 |
| A-162 | B-2 | C-2 | 80 | 15 | 5 |
| A-163 | B-2 | C-2 | 80 | 15 | 5 |
| A-164 | B-2 | C-2 | 80 | 15 | 5 |
| A-165 | B-2 | C-2 | 80 | 15 | 5 |
| A-166 | B-2 | C-2 | 80 | 15 | 5 |
| A-167 | B-2 | C-2 | 80 | 15 | 5 |
| A-168 | B-2 | C-2 | 80 | 15 | 5 |
| A-169 | B-2 | C-2 | 80 | 15 | 5 |
| A-170 | B-2 | C-2 | 80 | 15 | 5 |
| A-171 | B-2 | C-2 | 80 | 15 | 5 |
| A-172 | B-2 | C-2 | 80 | 15 | 5 |
| A-173 | B-2 | C-2 | 80 | 15 | 5 |
| A-174 | B-2 | C-2 | 80 | 15 | 5 |
| A-175 | B-2 | C-2 | 80 | 15 | 5 |
| A-176 | B-2 | C-2 | 80 | 15 | 5 |
| A-177 | B-2 | C-2 | 80 | 15 | 5 |
| A-178 | B-2 | C-2 | 80 | 15 | 5 |
| A-179 | B-2 | C-2 | 80 | 15 | 5 |
| A-180 | B-2 | C-2 | 80 | 15 | 5 |
| A-181 | B-2 | C-2 | 80 | 15 | 5 |
| A-182 | B-2 | C-2 | 80 | 15 | 5 |
| A-183 | B-2 | C-2 | 80 | 15 | 5 |
| A-190 | B-2 | C-2 | 80 | 15 | 5 |
| A-191 | B-2 | C-2 | 80 | 15 | 5 |
| A-192 | B-2 | C-2 | 80 | 15 | 5 |
| A-193 | B-2 | C-2 | 80 | 15 | 5 |
| A-194 | B-2 | C-2 | 80 | 15 | 5 |
| A-195 | B-2 | C-2 | 80 | 15 | 5 |
| A-196 | B-2 | C-2 | 80 | 15 | 5 |
| A-197 | B-2 | C-2 | 80 | 15 | 5 |
| A-198 | B-2 | C-2 | 80 | 15 | 5 |
| A-199 | B-2 | C-2 | 80 | 15 | 5 |

TABLE 24

| | | | Ratio of units (mol %) | | |
|---|---|---|---|---|---|
| A | B | C | A | B | C |
| A-200 | B-2 | C-2 | 80 | 15 | 5 |
| A-201 | B-2 | C-2 | 80 | 15 | 5 |
| A-202 | B-2 | C-2 | 80 | 15 | 5 |
| A-203 | B-2 | C-2 | 80 | 15 | 5 |
| A-204 | B-2 | C-2 | 80 | 15 | 5 |
| A-205 | B-2 | C-2 | 80 | 15 | 5 |
| A-206 | B-2 | C-2 | 80 | 15 | 5 |
| A-207 | B-2 | C-2 | 80 | 15 | 5 |
| A-208 | B-2 | C-2 | 80 | 15 | 5 |
| A-209 | B-2 | C-2 | 80 | 15 | 5 |
| A-210 | B-2 | C-2 | 80 | 15 | 5 |
| A-211 | B-2 | C-2 | 80 | 15 | 5 |
| A-212 | B-2 | C-2 | 80 | 15 | 5 |
| A-213 | B-2 | C-2 | 80 | 15 | 5 |
| A-220 | B-2 | C-2 | 80 | 15 | 5 |
| A-221 | B-2 | C-2 | 80 | 15 | 5 |
| A-222 | B-2 | C-2 | 80 | 15 | 5 |
| A-223 | B-2 | C-2 | 80 | 15 | 5 |
| A-224 | B-2 | C-2 | 80 | 15 | 5 |
| A-225 | B-2 | C-2 | 80 | 15 | 5 |
| A-226 | B-2 | C-2 | 80 | 15 | 5 |
| A-227 | B-2 | C-2 | 80 | 15 | 5 |
| A-228 | B-2 | C-2 | 80 | 15 | 5 |
| A-229 | B-2 | C-2 | 80 | 15 | 5 |
| A-230 | B-2 | C-2 | 80 | 15 | 5 |
| A-231 | B-2 | C-2 | 80 | 15 | 5 |
| A-232 | B-2 | C-2 | 80 | 15 | 5 |
| A-233 | B-2 | C-2 | 80 | 15 | 5 |
| A-234 | B-2 | C-2 | 80 | 15 | 5 |
| A-235 | B-2 | C-2 | 80 | 15 | 5 |
| A-236 | B-2 | C-2 | 80 | 15 | 5 |
| A-237 | B-2 | C-2 | 80 | 15 | 5 |
| A-238 | B-2 | C-2 | 80 | 15 | 5 |
| A-239 | B-2 | C-2 | 80 | 15 | 5 |
| A-240 | B-2 | C-2 | 80 | 15 | 5 |

TABLE 25

| | | | Ratio of units (mol %) | | |
|---|---|---|---|---|---|
| A | B | C | A | B | C |
| A-1 | B-2 | C-3 | 80 | 15 | 5 |
| A-2 | B-2 | C-3 | 80 | 15 | 5 |
| A-3 | B-2 | C-3 | 80 | 15 | 5 |
| A-4 | B-2 | C-3 | 80 | 15 | 5 |
| A-5 | B-2 | C-3 | 80 | 15 | 5 |
| A-6 | B-2 | C-3 | 80 | 15 | 5 |
| A-7 | B-2 | C-3 | 80 | 15 | 5 |
| A-8 | B-2 | C-3 | 80 | 15 | 5 |
| A-9 | B-2 | C-3 | 80 | 15 | 5 |
| A-10 | B-2 | C-3 | 80 | 15 | 5 |
| A-11 | B-2 | C-3 | 80 | 15 | 5 |
| A-12 | B-2 | C-3 | 80 | 15 | 5 |
| A-13 | B-2 | C-3 | 80 | 15 | 5 |
| A-14 | B-2 | C-3 | 80 | 15 | 5 |
| A-15 | B-2 | C-3 | 80 | 15 | 5 |
| A-16 | B-2 | C-3 | 80 | 15 | 5 |
| A-17 | B-2 | C-3 | 80 | 15 | 5 |
| A-18 | B-2 | C-3 | 80 | 15 | 5 |
| A-19 | B-2 | C-3 | 80 | 15 | 5 |
| A-20 | B-2 | C-3 | 80 | 15 | 5 |
| A-21 | B-2 | C-3 | 80 | 15 | 5 |
| A-22 | B-2 | C-3 | 80 | 15 | 5 |
| A-23 | B-2 | C-3 | 80 | 15 | 5 |
| A-24 | B-2 | C-3 | 80 | 15 | 5 |
| A-25 | B-2 | C-3 | 80 | 15 | 5 |
| A-26 | B-2 | C-3 | 80 | 15 | 5 |
| A-28 | B-2 | C-3 | 80 | 15 | 5 |
| A-29 | B-2 | C-3 | 80 | 15 | 5 |
| A-30 | B-2 | C-3 | 80 | 15 | 5 |
| A-31 | B-2 | C-3 | 80 | 15 | 5 |
| A-32 | B-2 | C-3 | 80 | 15 | 5 |
| A-33 | B-2 | C-3 | 80 | 15 | 5 |
| A-34 | B-2 | C-3 | 80 | 15 | 5 |
| A-35 | B-2 | C-3 | 80 | 15 | 5 |
| A-36 | B-2 | C-3 | 80 | 15 | 5 |
| A-37 | B-2 | C-3 | 80 | 15 | 5 |
| A-38 | B-2 | C-3 | 80 | 15 | 5 |
| A-39 | B-2 | C-3 | 80 | 15 | 5 |
| A-40 | B-2 | C-3 | 80 | 15 | 5 |
| A-41 | B-2 | C-3 | 80 | 15 | 5 |
| A-42 | B-2 | C-3 | 80 | 15 | 5 |
| A-43 | B-2 | C-3 | 80 | 15 | 5 |
| A-44 | B-2 | C-3 | 80 | 15 | 5 |
| A-45 | B-2 | C-3 | 80 | 15 | 5 |
| A-46 | B-2 | C-3 | 80 | 15 | 5 |
| A-47 | B-2 | C-3 | 80 | 15 | 5 |
| A-49 | B-2 | C-3 | 80 | 15 | 5 |
| A-50 | B-2 | C-3 | 80 | 15 | 5 |
| A-51 | B-2 | C-3 | 80 | 15 | 5 |
| A-52 | B-2 | C-3 | 80 | 15 | 5 |
| A-53 | B-2 | C-3 | 80 | 15 | 5 |
| A-54 | B-2 | C-3 | 80 | 15 | 5 |
| A-55 | B-2 | C-3 | 80 | 15 | 5 |
| A-56 | B-2 | C-3 | 80 | 15 | 5 |
| A-57 | B-2 | C-3 | 80 | 15 | 5 |
| A-58 | B-2 | C-3 | 80 | 15 | 5 |
| A-59 | B-2 | C-3 | 80 | 15 | 5 |
| A-60 | B-2 | C-3 | 80 | 15 | 5 |

TABLE 26

| | | | Ratio of units (mol %) | | |
|---|---|---|---|---|---|
| A | B | C | A | B | C |
| A-61 | B-2 | C-3 | 80 | 15 | 5 |
| A-62 | B-2 | C-3 | 80 | 15 | 5 |
| A-63 | B-2 | C-3 | 80 | 15 | 5 |
| A-64 | B-2 | C-3 | 80 | 15 | 5 |
| A-65 | B-2 | C-3 | 80 | 15 | 5 |
| A-66 | B-2 | C-3 | 80 | 15 | 5 |
| A-67 | B-2 | C-3 | 80 | 15 | 5 |
| A-68 | B-2 | C-3 | 80 | 15 | 5 |
| A-69 | B-2 | C-3 | 80 | 15 | 5 |
| A-70 | B-2 | C-3 | 80 | 15 | 5 |
| A-71 | B-2 | C-3 | 80 | 15 | 5 |
| A-72 | B-2 | C-3 | 80 | 15 | 5 |
| A-73 | B-2 | C-3 | 80 | 15 | 5 |
| A-74 | B-2 | C-3 | 80 | 15 | 5 |
| A-75 | B-2 | C-3 | 80 | 15 | 5 |
| A-76 | B-2 | C-3 | 80 | 15 | 5 |
| A-77 | B-2 | C-3 | 80 | 15 | 5 |
| A-78 | B-2 | C-3 | 80 | 15 | 5 |
| A-79 | B-2 | C-3 | 80 | 15 | 5 |
| A-80 | B-2 | C-3 | 80 | 15 | 5 |
| A-81 | B-2 | C-3 | 80 | 15 | 5 |
| A-82 | B-2 | C-3 | 80 | 15 | 5 |
| A-83 | B-2 | C-3 | 80 | 15 | 5 |
| A-84 | B-2 | C-3 | 80 | 15 | 5 |
| A-85 | B-2 | C-3 | 80 | 15 | 5 |
| A-86 | B-2 | C-3 | 80 | 15 | 5 |
| A-87 | B-2 | C-3 | 80 | 15 | 5 |
| A-88 | B-2 | C-3 | 80 | 15 | 5 |
| A-89 | B-2 | C-3 | 80 | 15 | 5 |
| A-90 | B-2 | C-3 | 80 | 15 | 5 |
| A-91 | B-2 | C-3 | 80 | 15 | 5 |
| A-92 | B-2 | C-3 | 80 | 15 | 5 |
| A-93 | B-2 | C-3 | 80 | 15 | 5 |
| A-94 | B-2 | C-3 | 80 | 15 | 5 |
| A-95 | B-2 | C-3 | 80 | 15 | 5 |
| A-96 | B-2 | C-3 | 80 | 15 | 5 |
| A-100 | B-2 | C-3 | 80 | 15 | 5 |
| A-101 | B-2 | C-3 | 80 | 15 | 5 |
| A-102 | B-2 | C-3 | 80 | 15 | 5 |
| A-103 | B-2 | C-3 | 80 | 15 | 5 |
| A-104 | B-2 | C-3 | 80 | 15 | 5 |
| A-105 | B-2 | C-3 | 80 | 15 | 5 |
| A-106 | B-2 | C-3 | 80 | 15 | 5 |
| A-107 | B-2 | C-3 | 80 | 15 | 5 |
| A-108 | B-2 | C-3 | 80 | 15 | 5 |
| A-109 | B-2 | C-3 | 80 | 15 | 5 |
| A-110 | B-2 | C-3 | 80 | 15 | 5 |
| A-111 | B-2 | C-3 | 80 | 15 | 5 |
| A-112 | B-2 | C-3 | 80 | 15 | 5 |
| A-113 | B-2 | C-3 | 80 | 15 | 5 |
| A-114 | B-2 | C-3 | 80 | 15 | 5 |
| A-115 | B-2 | C-3 | 80 | 15 | 5 |
| A-116 | B-2 | C-3 | 80 | 15 | 5 |
| A-117 | B-2 | C-3 | 80 | 15 | 5 |
| A-118 | B-2 | C-3 | 80 | 15 | 5 |
| A-119 | B-2 | C-3 | 80 | 15 | 5 |
| A-120 | B-2 | C-3 | 80 | 15 | 5 |
| A-121 | B-2 | C-3 | 80 | 15 | 5 |
| A-122 | B-2 | C-3 | 80 | 15 | 5 |
| A-123 | B-2 | C-3 | 80 | 15 | 5 |

TABLE 27

| | | | Ratio of units (mol %) | | |
|---|---|---|---|---|---|
| A | B | C | A | B | C |
| A-124 | B-2 | C-3 | 80 | 15 | 5 |
| A-125 | B-2 | C-3 | 80 | 15 | 5 |
| A-126 | B-2 | C-3 | 80 | 15 | 5 |
| A-127 | B-2 | C-3 | 80 | 15 | 5 |
| A-128 | B-2 | C-3 | 80 | 15 | 5 |
| A-129 | B-2 | C-3 | 80 | 15 | 5 |
| A-130 | B-2 | C-3 | 80 | 15 | 5 |
| A-131 | B-2 | C-3 | 80 | 15 | 5 |
| A-132 | B-2 | C-3 | 80 | 15 | 5 |
| A-133 | B-2 | C-3 | 80 | 15 | 5 |
| A-134 | B-2 | C-3 | 80 | 15 | 5 |
| A-135 | B-2 | C-3 | 80 | 15 | 5 |
| A-136 | B-2 | C-3 | 80 | 15 | 5 |
| A-137 | B-2 | C-3 | 80 | 15 | 5 |
| A-138 | B-2 | C-3 | 80 | 15 | 5 |
| A-139 | B-2 | C-3 | 80 | 15 | 5 |
| A-140 | B-2 | C-3 | 80 | 15 | 5 |
| A-141 | B-2 | C-3 | 80 | 15 | 5 |
| A-142 | B-2 | C-3 | 80 | 15 | 5 |
| A-143 | B-2 | C-3 | 80 | 15 | 5 |
| A-144 | B-2 | C-3 | 80 | 15 | 5 |
| A-145 | B-2 | C-3 | 80 | 15 | 5 |
| A-146 | B-2 | C-3 | 80 | 15 | 5 |
| A-147 | B-2 | C-3 | 80 | 15 | 5 |
| A-160 | B-2 | C-3 | 80 | 15 | 5 |
| A-161 | B-2 | C-3 | 80 | 15 | 5 |
| A-162 | B-2 | C-3 | 80 | 15 | 5 |
| A-163 | B-2 | C-3 | 80 | 15 | 5 |
| A-164 | B-2 | C-3 | 80 | 15 | 5 |
| A-165 | B-2 | C-3 | 80 | 15 | 5 |
| A-166 | B-2 | C-3 | 80 | 15 | 5 |
| A-167 | B-2 | C-3 | 80 | 15 | 5 |
| A-168 | B-2 | C-3 | 80 | 15 | 5 |
| A-169 | B-2 | C-3 | 80 | 15 | 5 |
| A-170 | B-2 | C-3 | 80 | 15 | 5 |
| A-171 | B-2 | C-3 | 80 | 15 | 5 |
| A-172 | B-2 | C-3 | 80 | 15 | 5 |
| A-173 | B-2 | C-3 | 80 | 15 | 5 |
| A-174 | B-2 | C-3 | 80 | 15 | 5 |
| A-175 | B-2 | C-3 | 80 | 15 | 5 |
| A-176 | B-2 | C-3 | 80 | 15 | 5 |
| A-177 | B-2 | C-3 | 80 | 15 | 5 |
| A-178 | B-2 | C-3 | 80 | 15 | 5 |
| A-179 | B-2 | C-3 | 80 | 15 | 5 |
| A-180 | B-2 | C-3 | 80 | 15 | 5 |
| A-181 | B-2 | C-3 | 80 | 15 | 5 |
| A-182 | B-2 | C-3 | 80 | 15 | 5 |
| A-183 | B-2 | C-3 | 80 | 15 | 5 |
| A-190 | B-2 | C-3 | 80 | 15 | 5 |
| A-191 | B-2 | C-3 | 80 | 15 | 5 |
| A-192 | B-2 | C-3 | 80 | 15 | 5 |
| A-193 | B-2 | C-3 | 80 | 15 | 5 |
| A-194 | B-2 | C-3 | 80 | 15 | 5 |
| A-195 | B-2 | C-3 | 80 | 15 | 5 |
| A-196 | B-2 | C-3 | 80 | 15 | 5 |
| A-197 | B-2 | C-3 | 80 | 15 | 5 |
| A-198 | B-2 | C-3 | 80 | 15 | 5 |
| A-199 | B-2 | C-3 | 80 | 15 | 5 |

TABLE 28

| | | | Ratio of units (mol %) | | |
|---|---|---|---|---|---|
| A | B | C | A | B | C |
| A-200 | B-2 | C-3 | 80 | 15 | 5 |
| A-201 | B-2 | C-3 | 80 | 15 | 5 |
| A-202 | B-2 | C-3 | 80 | 15 | 5 |
| A-203 | B-2 | C-3 | 80 | 15 | 5 |
| A-204 | B-2 | C-3 | 80 | 15 | 5 |
| A-205 | B-2 | C-3 | 80 | 15 | 5 |
| A-206 | B-2 | C-3 | 80 | 15 | 5 |
| A-207 | B-2 | C-3 | 80 | 15 | 5 |
| A-208 | B-2 | C-3 | 80 | 15 | 5 |
| A-209 | B-2 | C-3 | 80 | 15 | 5 |
| A-210 | B-2 | C-3 | 80 | 15 | 5 |
| A-211 | B-2 | C-3 | 80 | 15 | 5 |
| A-212 | B-2 | C-3 | 80 | 15 | 5 |
| A-213 | B-2 | C-3 | 80 | 15 | 5 |
| A-220 | B-2 | C-3 | 80 | 15 | 5 |
| A-221 | B-2 | C-3 | 80 | 15 | 5 |
| A-222 | B-2 | C-3 | 80 | 15 | 5 |
| A-223 | B-2 | C-3 | 80 | 15 | 5 |
| A-224 | B-2 | C-3 | 80 | 15 | 5 |
| A-225 | B-2 | C-3 | 80 | 15 | 5 |
| A-226 | B-2 | C-3 | 80 | 15 | 5 |
| A-227 | B-2 | C-3 | 80 | 15 | 5 |
| A-228 | B-2 | C-3 | 80 | 15 | 5 |
| A-229 | B-2 | C-3 | 80 | 15 | 5 |
| A-230 | B-2 | C-3 | 80 | 15 | 5 |
| A-231 | B-2 | C-3 | 80 | 15 | 5 |
| A-232 | B-2 | C-3 | 80 | 15 | 5 |
| A-233 | B-2 | C-3 | 80 | 15 | 5 |
| A-234 | B-2 | C-3 | 80 | 15 | 5 |
| A-235 | B-2 | C-3 | 80 | 15 | 5 |
| A-236 | B-2 | C-3 | 80 | 15 | 5 |
| A-237 | B-2 | C-3 | 80 | 15 | 5 |
| A-238 | B-2 | C-3 | 80 | 15 | 5 |
| A-239 | B-2 | C-3 | 80 | 15 | 5 |
| A-240 | B-2 | C-3 | 80 | 15 | 5 |

TABLE 29

| | | | Ratio of units (mol %) | | |
|---|---|---|---|---|---|
| A | B | C | A | B | C |
| A-1 | B-2 | C-4 | 80 | 15 | 5 |
| A-2 | B-2 | C-4 | 80 | 15 | 5 |
| A-3 | B-2 | C-4 | 80 | 15 | 5 |
| A-4 | B-2 | C-4 | 80 | 15 | 5 |
| A-5 | B-2 | C-4 | 80 | 15 | 5 |
| A-6 | B-2 | C-4 | 80 | 15 | 5 |
| A-7 | B-2 | C-4 | 80 | 15 | 5 |
| A-8 | B-2 | C-4 | 80 | 15 | 5 |
| A-9 | B-2 | C-4 | 80 | 15 | 5 |
| A-10 | B-2 | C-4 | 80 | 15 | 5 |
| A-11 | B-2 | C-4 | 80 | 15 | 5 |
| A-12 | B-2 | C-4 | 80 | 15 | 5 |
| A-13 | B-2 | C-4 | 80 | 15 | 5 |
| A-14 | B-2 | C-4 | 80 | 15 | 5 |
| A-15 | B-2 | C-4 | 80 | 15 | 5 |
| A-16 | B-2 | C-4 | 80 | 15 | 5 |
| A-17 | B-2 | C-4 | 80 | 15 | 5 |
| A-18 | B-2 | C-4 | 80 | 15 | 5 |
| A-19 | B-2 | C-4 | 80 | 15 | 5 |
| A-20 | B-2 | C-4 | 80 | 15 | 5 |
| A-21 | B-2 | C-4 | 80 | 15 | 5 |
| A-22 | B-2 | C-4 | 80 | 15 | 5 |
| A-23 | B-2 | C-4 | 80 | 15 | 5 |
| A-24 | B-2 | C-4 | 80 | 15 | 5 |
| A-25 | B-2 | C-4 | 80 | 15 | 5 |
| A-26 | B-2 | C-4 | 80 | 15 | 5 |
| A-28 | B-2 | C-4 | 80 | 15 | 5 |
| A-29 | B-2 | C-4 | 80 | 15 | 5 |
| A-30 | B-2 | C-4 | 80 | 15 | 5 |
| A-31 | B-2 | C-4 | 80 | 15 | 5 |
| A-32 | B-2 | C-4 | 80 | 15 | 5 |
| A-33 | B-2 | C-4 | 80 | 15 | 5 |
| A-34 | B-2 | C-4 | 80 | 15 | 5 |
| A-35 | B-2 | C-4 | 80 | 15 | 5 |
| A-36 | B-2 | C-4 | 80 | 15 | 5 |
| A-37 | B-2 | C-4 | 80 | 15 | 5 |
| A-38 | B-2 | C-4 | 80 | 15 | 5 |
| A-39 | B-2 | C-4 | 80 | 15 | 5 |
| A-40 | B-2 | C-4 | 80 | 15 | 5 |
| A-41 | B-2 | C-4 | 80 | 15 | 5 |
| A-42 | B-2 | C-4 | 80 | 15 | 5 |
| A-43 | B-2 | C-4 | 80 | 15 | 5 |
| A-44 | B-2 | C-4 | 80 | 15 | 5 |
| A-45 | B-2 | C-4 | 80 | 15 | 5 |
| A-46 | B-2 | C-4 | 80 | 15 | 5 |
| A-47 | B-2 | C-4 | 80 | 15 | 5 |
| A-49 | B-2 | C-4 | 80 | 15 | 5 |
| A-50 | B-2 | C-4 | 80 | 15 | 5 |
| A-51 | B-2 | C-4 | 80 | 15 | 5 |
| A-52 | B-2 | C-4 | 80 | 15 | 5 |
| A-53 | B-2 | C-4 | 80 | 15 | 5 |
| A-54 | B-2 | C-4 | 80 | 15 | 5 |
| A-55 | B-2 | C-4 | 80 | 15 | 5 |
| A-56 | B-2 | C-4 | 80 | 15 | 5 |
| A-57 | B-2 | C-4 | 80 | 15 | 5 |
| A-58 | B-2 | C-4 | 80 | 15 | 5 |
| A-59 | B-2 | C-4 | 80 | 15 | 5 |
| A-60 | B-2 | C-4 | 80 | 15 | 5 |

TABLE 30

| | | | Ratio of units (mol %) | | |
|---|---|---|---|---|---|
| A | B | C | A | B | C |
| A-61 | B-2 | C-4 | 80 | 15 | 5 |
| A-62 | B-2 | C-4 | 80 | 15 | 5 |
| A-63 | B-2 | C-4 | 80 | 15 | 5 |
| A-64 | B-2 | C-4 | 80 | 15 | 5 |
| A-65 | B-2 | C-4 | 80 | 15 | 5 |
| A-66 | B-2 | C-4 | 80 | 15 | 5 |
| A-67 | B-2 | C-4 | 80 | 15 | 5 |
| A-68 | B-2 | C-4 | 80 | 15 | 5 |
| A-69 | B-2 | C-4 | 80 | 15 | 5 |
| A-70 | B-2 | C-4 | 80 | 15 | 5 |
| A-71 | B-2 | C-4 | 80 | 15 | 5 |
| A-72 | B-2 | C-4 | 80 | 15 | 5 |
| A-73 | B-2 | C-4 | 80 | 15 | 5 |
| A-74 | B-2 | C-4 | 80 | 15 | 5 |
| A-75 | B-2 | C-4 | 80 | 15 | 5 |
| A-76 | B-2 | C-4 | 80 | 15 | 5 |
| A-77 | B-2 | C-4 | 80 | 15 | 5 |
| A-78 | B-2 | C-4 | 80 | 15 | 5 |
| A-79 | B-2 | C-4 | 80 | 15 | 5 |
| A-80 | B-2 | C-4 | 80 | 15 | 5 |
| A-81 | B-2 | C-4 | 80 | 15 | 5 |
| A-82 | B-2 | C-4 | 80 | 15 | 5 |
| A-83 | B-2 | C-4 | 80 | 15 | 5 |
| A-84 | B-2 | C-4 | 80 | 15 | 5 |
| A-85 | B-2 | C-4 | 80 | 15 | 5 |
| A-86 | B-2 | C-4 | 80 | 15 | 5 |
| A-87 | B-2 | C-4 | 80 | 15 | 5 |
| A-88 | B-2 | C-4 | 80 | 15 | 5 |
| A-89 | B-2 | C-4 | 80 | 15 | 5 |
| A-90 | B-2 | C-4 | 80 | 15 | 5 |
| A-91 | B-2 | C-4 | 80 | 15 | 5 |
| A-92 | B-2 | C-4 | 80 | 15 | 5 |
| A-93 | B-2 | C-4 | 80 | 15 | 5 |
| A-94 | B-2 | C-4 | 80 | 15 | 5 |
| A-95 | B-2 | C-4 | 80 | 15 | 5 |
| A-96 | B-2 | C-4 | 80 | 15 | 5 |
| A-100 | B-2 | C-4 | 80 | 15 | 5 |
| A-101 | B-2 | C-4 | 80 | 15 | 5 |
| A-102 | B-2 | C-4 | 80 | 15 | 5 |
| A-103 | B-2 | C-4 | 80 | 15 | 5 |
| A-104 | B-2 | C-4 | 80 | 15 | 5 |
| A-105 | B-2 | C-4 | 80 | 15 | 5 |
| A-106 | B-2 | C-4 | 80 | 15 | 5 |
| A-107 | B-2 | C-4 | 80 | 15 | 5 |
| A-108 | B-2 | C-4 | 80 | 15 | 5 |
| A-109 | B-2 | C-4 | 80 | 15 | 5 |
| A-110 | B-2 | C-4 | 80 | 15 | 5 |
| A-111 | B-2 | C-4 | 80 | 15 | 5 |
| A-112 | B-2 | C-4 | 80 | 15 | 5 |
| A-113 | B-2 | C-4 | 80 | 15 | 5 |
| A-114 | B-2 | C-4 | 80 | 15 | 5 |
| A-115 | B-2 | C-4 | 80 | 15 | 5 |
| A-116 | B-2 | C-4 | 80 | 15 | 5 |
| A-117 | B-2 | C-4 | 80 | 15 | 5 |
| A-118 | B-2 | C-4 | 80 | 15 | 5 |
| A-119 | B-2 | C-4 | 80 | 15 | 5 |
| A-120 | B-2 | C-4 | 80 | 15 | 5 |
| A-121 | B-2 | C-4 | 80 | 15 | 5 |
| A-122 | B-2 | C-4 | 80 | 15 | 5 |
| A-123 | B-2 | C-4 | 80 | 15 | 5 |

TABLE 31

| | | | Ratio of units (mol %) | | |
|---|---|---|---|---|---|
| A | B | C | A | B | C |
| A-124 | B-2 | C-4 | 80 | 15 | 5 |
| A-125 | B-2 | C-4 | 80 | 15 | 5 |
| A-126 | B-2 | C-4 | 80 | 15 | 5 |
| A-127 | B-2 | C-4 | 80 | 15 | 5 |
| A-128 | B-2 | C-4 | 80 | 15 | 5 |
| A-129 | B-2 | C-4 | 80 | 15 | 5 |
| A-130 | B-2 | C-4 | 80 | 15 | 5 |
| A-131 | B-2 | C-4 | 80 | 15 | 5 |
| A-132 | B-2 | C-4 | 80 | 15 | 5 |
| A-133 | B-2 | C-4 | 80 | 15 | 5 |
| A-134 | B-2 | C-4 | 80 | 15 | 5 |
| A-135 | B-2 | C-4 | 80 | 15 | 5 |
| A-136 | B-2 | C-4 | 80 | 15 | 5 |
| A-137 | B-2 | C-4 | 80 | 15 | 5 |
| A-138 | B-2 | C-4 | 80 | 15 | 5 |
| A-139 | B-2 | C-4 | 80 | 15 | 5 |
| A-140 | B-2 | C-4 | 80 | 15 | 5 |
| A-141 | B-2 | C-4 | 80 | 15 | 5 |
| A-142 | B-2 | C-4 | 80 | 15 | 5 |
| A-143 | B-2 | C-4 | 80 | 15 | 5 |
| A-144 | B-2 | C-4 | 80 | 15 | 5 |
| A-145 | B-2 | C-4 | 80 | 15 | 5 |
| A-146 | B-2 | C-4 | 80 | 15 | 5 |
| A-147 | B-2 | C-4 | 80 | 15 | 5 |
| A-160 | B-2 | C-4 | 80 | 15 | 5 |
| A-161 | B-2 | C-4 | 80 | 15 | 5 |
| A-162 | B-2 | C-4 | 80 | 15 | 5 |
| A-163 | B-2 | C-4 | 80 | 15 | 5 |
| A-164 | B-2 | C-4 | 80 | 15 | 5 |
| A-165 | B-2 | C-4 | 80 | 15 | 5 |
| A-166 | B-2 | C-4 | 80 | 15 | 5 |
| A-167 | B-2 | C-4 | 80 | 15 | 5 |
| A-168 | B-2 | C-4 | 80 | 15 | 5 |
| A-169 | B-2 | C-4 | 80 | 15 | 5 |
| A-170 | B-2 | C-4 | 80 | 15 | 5 |
| A-171 | B-2 | C-4 | 80 | 15 | 5 |
| A-172 | B-2 | C-4 | 80 | 15 | 5 |
| A-173 | B-2 | C-4 | 80 | 15 | 5 |
| A-174 | B-2 | C-4 | 80 | 15 | 5 |
| A-175 | B-2 | C-4 | 80 | 15 | 5 |
| A-176 | B-2 | C-4 | 80 | 15 | 5 |
| A-177 | B-2 | C-4 | 80 | 15 | 5 |
| A-178 | B-2 | C-4 | 80 | 15 | 5 |
| A-179 | B-2 | C-4 | 80 | 15 | 5 |
| A-180 | B-2 | C-4 | 80 | 15 | 5 |
| A-181 | B-2 | C-4 | 80 | 15 | 5 |
| A-182 | B-2 | C-4 | 80 | 15 | 5 |
| A-183 | B-2 | C-4 | 80 | 15 | 5 |
| A-190 | B-2 | C-4 | 80 | 15 | 5 |
| A-191 | B-2 | C-4 | 80 | 15 | 5 |
| A-192 | B-2 | C-4 | 80 | 15 | 5 |
| A-193 | B-2 | C-4 | 80 | 15 | 5 |
| A-194 | B-2 | C-4 | 80 | 15 | 5 |
| A-195 | B-2 | C-4 | 80 | 15 | 5 |
| A-196 | B-2 | C-4 | 80 | 15 | 5 |
| A-197 | B-2 | C-4 | 80 | 15 | 5 |
| A-198 | B-2 | C-4 | 80 | 15 | 5 |
| A-199 | B-2 | C-4 | 80 | 15 | 5 |

TABLE 32

| | | | Ratio of units (mol %) | | |
|---|---|---|---|---|---|
| A | B | C | A | B | C |
| A-200 | B-2 | C-4 | 80 | 15 | 5 |
| A-201 | B-2 | C-4 | 80 | 15 | 5 |
| A-202 | B-2 | C-4 | 80 | 15 | 5 |
| A-203 | B-2 | C-4 | 80 | 15 | 5 |
| A-204 | B-2 | C-4 | 80 | 15 | 5 |
| A-205 | B-2 | C-4 | 80 | 15 | 5 |
| A-206 | B-2 | C-4 | 80 | 15 | 5 |
| A-207 | B-2 | C-4 | 80 | 15 | 5 |
| A-208 | B-2 | C-4 | 80 | 15 | 5 |
| A-209 | B-2 | C-4 | 80 | 15 | 5 |
| A-210 | B-2 | C-4 | 80 | 15 | 5 |
| A-211 | B-2 | C-4 | 80 | 15 | 5 |
| A-212 | B-2 | C-4 | 80 | 15 | 5 |
| A-213 | B-2 | C-4 | 80 | 15 | 5 |
| A-220 | B-2 | C-4 | 80 | 15 | 5 |
| A-221 | B-2 | C-4 | 80 | 15 | 5 |
| A-222 | B-2 | C-4 | 80 | 15 | 5 |
| A-223 | B-2 | C-4 | 80 | 15 | 5 |
| A-224 | B-2 | C-4 | 80 | 15 | 5 |
| A-225 | B-2 | C-4 | 80 | 15 | 5 |
| A-226 | B-2 | C-4 | 80 | 15 | 5 |
| A-227 | B-2 | C-4 | 80 | 15 | 5 |
| A-228 | B-2 | C-4 | 80 | 15 | 5 |
| A-229 | B-2 | C-4 | 80 | 15 | 5 |
| A-230 | B-2 | C-4 | 80 | 15 | 5 |
| A-231 | B-2 | C-4 | 80 | 15 | 5 |
| A-232 | B-2 | C-4 | 80 | 15 | 5 |
| A-233 | B-2 | C-4 | 80 | 15 | 5 |
| A-234 | B-2 | C-4 | 80 | 15 | 5 |
| A-235 | B-2 | C-4 | 80 | 15 | 5 |
| A-236 | B-2 | C-4 | 80 | 15 | 5 |
| A-237 | B-2 | C-4 | 80 | 15 | 5 |
| A-238 | B-2 | C-4 | 80 | 15 | 5 |
| A-239 | B-2 | C-4 | 80 | 15 | 5 |
| A-240 | B-2 | C-4 | 80 | 15 | 5 |

TABLE 33

| | | | Ratio of units (mol %) | | |
|---|---|---|---|---|---|
| A | B | C | A | B | C |
| A-1 | B-3 | C-1 | 80 | 15 | 5 |
| A-2 | B-3 | C-1 | 80 | 15 | 5 |
| A-3 | B-3 | C-1 | 80 | 15 | 5 |
| A-4 | B-3 | C-1 | 80 | 15 | 5 |
| A-5 | B-3 | C-1 | 80 | 15 | 5 |
| A-6 | B-3 | C-1 | 80 | 15 | 5 |
| A-7 | B-3 | C-1 | 80 | 15 | 5 |
| A-8 | B-3 | C-1 | 80 | 15 | 5 |
| A-9 | B-3 | C-1 | 80 | 15 | 5 |
| A-10 | B-3 | C-1 | 80 | 15 | 5 |
| A-11 | B-3 | C-1 | 80 | 15 | 5 |
| A-12 | B-3 | C-1 | 80 | 15 | 5 |
| A-13 | B-3 | C-1 | 80 | 15 | 5 |
| A-14 | B-3 | C-1 | 80 | 15 | 5 |
| A-15 | B-3 | C-1 | 80 | 15 | 5 |
| A-16 | B-3 | C-1 | 80 | 15 | 5 |
| A-17 | B-3 | C-1 | 80 | 15 | 5 |
| A-18 | B-3 | C-1 | 80 | 15 | 5 |
| A-19 | B-3 | C-1 | 80 | 15 | 5 |
| A-20 | B-3 | C-1 | 80 | 15 | 5 |
| A-21 | B-3 | C-1 | 80 | 15 | 5 |
| A-22 | B-3 | C-1 | 80 | 15 | 5 |
| A-23 | B-3 | C-1 | 80 | 15 | 5 |
| A-24 | B-3 | C-1 | 80 | 15 | 5 |
| A-25 | B-3 | C-1 | 80 | 15 | 5 |
| A-26 | B-3 | C-1 | 80 | 15 | 5 |
| A-28 | B-3 | C-1 | 80 | 15 | 5 |
| A-29 | B-3 | C-1 | 80 | 15 | 5 |
| A-30 | B-3 | C-1 | 80 | 15 | 5 |
| A-31 | B-3 | C-1 | 80 | 15 | 5 |
| A-32 | B-3 | C-1 | 80 | 15 | 5 |
| A-33 | B-3 | C-1 | 80 | 15 | 5 |
| A-34 | B-3 | C-1 | 80 | 15 | 5 |
| A-35 | B-3 | C-1 | 80 | 15 | 5 |
| A-36 | B-3 | C-1 | 80 | 15 | 5 |
| A-37 | B-3 | C-1 | 80 | 15 | 5 |
| A-38 | B-3 | C-1 | 80 | 15 | 5 |
| A-39 | B-3 | C-1 | 80 | 15 | 5 |
| A-40 | B-3 | C-1 | 80 | 15 | 5 |
| A-41 | B-3 | C-1 | 80 | 15 | 5 |
| A-42 | B-3 | C-1 | 80 | 15 | 5 |
| A-43 | B-3 | C-1 | 80 | 15 | 5 |
| A-44 | B-3 | C-1 | 80 | 15 | 5 |
| A-45 | B-3 | C-1 | 80 | 15 | 5 |
| A-46 | B-3 | C-1 | 80 | 15 | 5 |
| A-47 | B-3 | C-1 | 80 | 15 | 5 |
| A-49 | B-3 | C-1 | 80 | 15 | 5 |
| A-50 | B-3 | C-1 | 80 | 15 | 5 |
| A-51 | B-3 | C-1 | 80 | 15 | 5 |
| A-52 | B-3 | C-1 | 80 | 15 | 5 |
| A-53 | B-3 | C-1 | 80 | 15 | 5 |
| A-54 | B-3 | C-1 | 80 | 15 | 5 |
| A-55 | B-3 | C-1 | 80 | 15 | 5 |
| A-56 | B-3 | C-1 | 80 | 15 | 5 |
| A-57 | B-3 | C-1 | 80 | 15 | 5 |
| A-58 | B-3 | C-1 | 80 | 15 | 5 |
| A-59 | B-3 | C-1 | 80 | 15 | 5 |
| A-60 | B-3 | C-1 | 80 | 15 | 5 |

TABLE 34

| | | | Ratio of units (mol %) | | |
|---|---|---|---|---|---|
| A | B | C | A | B | C |
| A-61 | B-3 | C-1 | 80 | 15 | 5 |
| A-62 | B-3 | C-1 | 80 | 15 | 5 |
| A-63 | B-3 | C-1 | 80 | 15 | 5 |
| A-64 | B-3 | C-1 | 80 | 15 | 5 |
| A-65 | B-3 | C-1 | 80 | 15 | 5 |
| A-66 | B-3 | C-1 | 80 | 15 | 5 |
| A-67 | B-3 | C-1 | 80 | 15 | 5 |
| A-68 | B-3 | C-1 | 80 | 15 | 5 |
| A-69 | B-3 | C-1 | 80 | 15 | 5 |
| A-70 | B-3 | C-1 | 80 | 15 | 5 |
| A-71 | B-3 | C-1 | 80 | 15 | 5 |
| A-72 | B-3 | C-1 | 80 | 15 | 5 |
| A-73 | B-3 | C-1 | 80 | 15 | 5 |
| A-74 | B-3 | C-1 | 80 | 15 | 5 |
| A-75 | B-3 | C-1 | 80 | 15 | 5 |
| A-76 | B-3 | C-1 | 80 | 15 | 5 |
| A-77 | B-3 | C-1 | 80 | 15 | 5 |
| A-78 | B-3 | C-1 | 80 | 15 | 5 |
| A-79 | B-3 | C-1 | 80 | 15 | 5 |
| A-80 | B-3 | C-1 | 80 | 15 | 5 |
| A-81 | B-3 | C-1 | 80 | 15 | 5 |
| A-82 | B-3 | C-1 | 80 | 15 | 5 |
| A-83 | B-3 | C-1 | 80 | 15 | 5 |
| A-84 | B-3 | C-1 | 80 | 15 | 5 |
| A-85 | B-3 | C-1 | 80 | 15 | 5 |
| A-86 | B-3 | C-1 | 80 | 15 | 5 |
| A-87 | B-3 | C-1 | 80 | 15 | 5 |
| A-88 | B-3 | C-1 | 80 | 15 | 5 |
| A-89 | B-3 | C-1 | 80 | 15 | 5 |
| A-90 | B-3 | C-1 | 80 | 15 | 5 |
| A-91 | B-3 | C-1 | 80 | 15 | 5 |
| A-92 | B-3 | C-1 | 80 | 15 | 5 |
| A-93 | B-3 | C-1 | 80 | 15 | 5 |
| A-94 | B-3 | C-1 | 80 | 15 | 5 |
| A-95 | B-3 | C-1 | 80 | 15 | 5 |
| A-96 | B-3 | C-1 | 80 | 15 | 5 |
| A-100 | B-3 | C-1 | 80 | 15 | 5 |
| A-101 | B-3 | C-1 | 80 | 15 | 5 |
| A-102 | B-3 | C-1 | 80 | 15 | 5 |
| A-103 | B-3 | C-1 | 80 | 15 | 5 |
| A-104 | B-3 | C-1 | 80 | 15 | 5 |
| A-105 | B-3 | C-1 | 80 | 15 | 5 |
| A-106 | B-3 | C-1 | 80 | 15 | 5 |
| A-107 | B-3 | C-1 | 80 | 15 | 5 |
| A-108 | B-3 | C-1 | 80 | 15 | 5 |
| A-109 | B-3 | C-1 | 80 | 15 | 5 |
| A-110 | B-3 | C-1 | 80 | 15 | 5 |
| A-111 | B-3 | C-1 | 80 | 15 | 5 |
| A-112 | B-3 | C-1 | 80 | 15 | 5 |
| A-113 | B-3 | C-1 | 80 | 15 | 5 |
| A-114 | B-3 | C-1 | 80 | 15 | 5 |
| A-115 | B-3 | C-1 | 80 | 15 | 5 |
| A-116 | B-3 | C-1 | 80 | 15 | 5 |
| A-117 | B-3 | C-1 | 80 | 15 | 5 |
| A-118 | B-3 | C-1 | 80 | 15 | 5 |
| A-119 | B-3 | C-1 | 80 | 15 | 5 |
| A-120 | B-3 | C-1 | 80 | 15 | 5 |
| A-121 | B-3 | C-1 | 80 | 15 | 5 |
| A-122 | B-3 | C-1 | 80 | 15 | 5 |
| A-123 | B-3 | C-1 | 80 | 15 | 5 |

TABLE 35

| | | | Ratio of | | |
|---|---|---|---|---|---|
| | | | units | | |
| | | | (mol %) | | |
| A | B | C | A | B | C |
| A-124 | B-3 | C-1 | 80 | 15 | 5 |
| A-125 | B-3 | C-1 | 80 | 15 | 5 |
| A-126 | B-3 | C-1 | 80 | 15 | 5 |
| A-127 | B-3 | C-1 | 80 | 15 | 5 |
| A-128 | B-3 | C-1 | 80 | 15 | 5 |
| A-129 | B-3 | C-1 | 80 | 15 | 5 |
| A-130 | B-3 | C-1 | 80 | 15 | 5 |
| A-131 | B-3 | C-1 | 80 | 15 | 5 |
| A-132 | B-3 | C-1 | 80 | 15 | 5 |
| A-133 | B-3 | C-1 | 80 | 15 | 5 |
| A-134 | B-3 | C-1 | 80 | 15 | 5 |
| A-135 | B-3 | C-1 | 80 | 15 | 5 |
| A-136 | B-3 | C-1 | 80 | 15 | 5 |
| A-137 | B-3 | C-1 | 80 | 15 | 5 |
| A-138 | B-3 | C-1 | 80 | 15 | 5 |
| A-139 | B-3 | C-1 | 80 | 15 | 5 |
| A-140 | B-3 | C-1 | 80 | 15 | 5 |
| A-141 | B-3 | C-1 | 80 | 15 | 5 |
| A-142 | B-3 | C-1 | 80 | 15 | 5 |
| A-143 | B-3 | C-1 | 80 | 15 | 5 |
| A-144 | B-3 | C-1 | 80 | 15 | 5 |
| A-145 | B-3 | C-1 | 80 | 15 | 5 |
| A-146 | B-3 | C-1 | 80 | 15 | 5 |
| A-147 | B-3 | C-1 | 80 | 15 | 5 |
| A-160 | B-3 | C-1 | 80 | 15 | 5 |
| A-161 | B-3 | C-1 | 80 | 15 | 5 |
| A-162 | B-3 | C-1 | 80 | 15 | 5 |
| A-163 | B-3 | C-1 | 80 | 15 | 5 |
| A-164 | B-3 | C-1 | 80 | 15 | 5 |
| A-165 | B-3 | C-1 | 80 | 15 | 5 |
| A-166 | B-3 | C-1 | 80 | 15 | 5 |
| A-167 | B-3 | C-1 | 80 | 15 | 5 |
| A-168 | B-3 | C-1 | 80 | 15 | 5 |
| A-169 | B-3 | C-1 | 80 | 15 | 5 |
| A-170 | B-3 | C-1 | 80 | 15 | 5 |
| A-171 | B-3 | C-1 | 80 | 15 | 5 |
| A-172 | B-3 | C-1 | 80 | 15 | 5 |
| A-173 | B-3 | C-1 | 80 | 15 | 5 |
| A-174 | B-3 | C-1 | 80 | 15 | 5 |
| A-175 | B-3 | C-1 | 80 | 15 | 5 |
| A-176 | B-3 | C-1 | 80 | 15 | 5 |
| A-177 | B-3 | C-1 | 80 | 15 | 5 |
| A-178 | B-3 | C-1 | 80 | 15 | 5 |
| A-179 | B-3 | C-1 | 80 | 15 | 5 |
| A-180 | B-3 | C-1 | 80 | 15 | 5 |
| A-181 | B-3 | C-1 | 80 | 15 | 5 |
| A-182 | B-3 | C-1 | 80 | 15 | 5 |
| A-183 | B-3 | C-1 | 80 | 15 | 5 |
| A-190 | B-3 | C-1 | 80 | 15 | 5 |
| A-191 | B-3 | C-1 | 80 | 15 | 5 |
| A-192 | B-3 | C-1 | 80 | 15 | 5 |
| A-193 | B-3 | C-1 | 80 | 15 | 5 |
| A-194 | B-3 | C-1 | 80 | 15 | 5 |
| A-195 | B-3 | C-1 | 80 | 15 | 5 |
| A-196 | B-3 | C-1 | 80 | 15 | 5 |
| A-197 | B-3 | C-1 | 80 | 15 | 5 |
| A-198 | B-3 | C-1 | 80 | 15 | 5 |
| A-199 | B-3 | C-1 | 80 | 15 | 5 |

TABLE 36

| | | | Ratio of | | |
|---|---|---|---|---|---|
| | | | units | | |
| | | | (mol %) | | |
| A | B | C | A | B | C |
| A-200 | B-3 | C-1 | 80 | 15 | 5 |
| A-201 | B-3 | C-1 | 80 | 15 | 5 |
| A-202 | B-3 | C-1 | 80 | 15 | 5 |
| A-203 | B-3 | C-1 | 80 | 15 | 5 |
| A-204 | B-3 | C-1 | 80 | 15 | 5 |
| A-205 | B-3 | C-1 | 80 | 15 | 5 |
| A-206 | B-3 | C-1 | 80 | 15 | 5 |
| A-207 | B-3 | C-1 | 80 | 15 | 5 |
| A-208 | B-3 | C-1 | 80 | 15 | 5 |
| A-209 | B-3 | C-1 | 80 | 15 | 5 |
| A-210 | B-3 | C-1 | 80 | 15 | 5 |
| A-211 | B-3 | C-1 | 80 | 15 | 5 |
| A-212 | B-3 | C-1 | 80 | 15 | 5 |
| A-213 | B-3 | C-1 | 80 | 15 | 5 |
| A-220 | B-3 | C-1 | 80 | 15 | 5 |
| A-221 | B-3 | C-1 | 80 | 15 | 5 |
| A-222 | B-3 | C-1 | 80 | 15 | 5 |
| A-223 | B-3 | C-1 | 80 | 15 | 5 |
| A-224 | B-3 | C-1 | 80 | 15 | 5 |
| A-225 | B-3 | C-1 | 80 | 15 | 5 |
| A-226 | B-3 | C-1 | 80 | 15 | 5 |
| A-227 | B-3 | C-1 | 80 | 15 | 5 |
| A-228 | B-3 | C-1 | 80 | 15 | 5 |
| A-229 | B-3 | C-1 | 80 | 15 | 5 |
| A-230 | B-3 | C-1 | 80 | 15 | 5 |
| A-231 | B-3 | C-1 | 80 | 15 | 5 |
| A-232 | B-3 | C-1 | 80 | 15 | 5 |
| A-233 | B-3 | C-1 | 80 | 15 | 5 |
| A-234 | B-3 | C-1 | 80 | 15 | 5 |
| A-235 | B-3 | C-1 | 80 | 15 | 5 |
| A-236 | B-3 | C-1 | 80 | 15 | 5 |
| A-237 | B-3 | C-1 | 80 | 15 | 5 |
| A-238 | B-3 | C-1 | 80 | 15 | 5 |
| A-239 | B-3 | C-1 | 80 | 15 | 5 |
| A-240 | B-3 | C-1 | 80 | 15 | 5 |

TABLE 37

| | | | Ratio of | | |
|---|---|---|---|---|---|
| | | | units | | |
| | | | (mol %) | | |
| A | B | C | A | B | C |
| A-1 | B-3 | C-2 | 80 | 15 | 5 |
| A-2 | B-3 | C-2 | 80 | 15 | 5 |
| A-3 | B-3 | C-2 | 80 | 15 | 5 |
| A-4 | B-3 | C-2 | 80 | 15 | 5 |
| A-5 | B-3 | C-2 | 80 | 15 | 5 |
| A-6 | B-3 | C-2 | 80 | 15 | 5 |
| A-7 | B-3 | C-2 | 80 | 15 | 5 |
| A-8 | B-3 | C-2 | 80 | 15 | 5 |
| A-9 | B-3 | C-2 | 80 | 15 | 5 |
| A-10 | B-3 | C-2 | 80 | 15 | 5 |
| A-11 | B-3 | C-2 | 80 | 15 | 5 |
| A-12 | B-3 | C-2 | 80 | 15 | 5 |
| A-13 | B-3 | C-2 | 80 | 15 | 5 |
| A-14 | B-3 | C-2 | 80 | 15 | 5 |
| A-15 | B-3 | C-2 | 80 | 15 | 5 |
| A-16 | B-3 | C-2 | 80 | 15 | 5 |
| A-17 | B-3 | C-2 | 80 | 15 | 5 |
| A-18 | B-3 | C-2 | 80 | 15 | 5 |
| A-19 | B-3 | C-2 | 80 | 15 | 5 |
| A-20 | B-3 | C-2 | 80 | 15 | 5 |
| A-21 | B-3 | C-2 | 80 | 15 | 5 |
| A-22 | B-3 | C-2 | 80 | 15 | 5 |
| A-23 | B-3 | C-2 | 80 | 15 | 5 |
| A-24 | B-3 | C-2 | 80 | 15 | 5 |
| A-25 | B-3 | C-2 | 80 | 15 | 5 |
| A-26 | B-3 | C-2 | 80 | 15 | 5 |
| A-28 | B-3 | C-2 | 80 | 15 | 5 |
| A-29 | B-3 | C-2 | 80 | 15 | 5 |
| A-30 | B-3 | C-2 | 80 | 15 | 5 |
| A-31 | B-3 | C-2 | 80 | 15 | 5 |
| A-32 | B-3 | C-2 | 80 | 15 | 5 |
| A-33 | B-3 | C-2 | 80 | 15 | 5 |
| A-34 | B-3 | C-2 | 80 | 15 | 5 |
| A-35 | B-3 | C-2 | 80 | 15 | 5 |
| A-36 | B-3 | C-2 | 80 | 15 | 5 |
| A-37 | B-3 | C-2 | 80 | 15 | 5 |
| A-38 | B-3 | C-2 | 80 | 15 | 5 |
| A-39 | B-3 | C-2 | 80 | 15 | 5 |
| A-40 | B-3 | C-2 | 80 | 15 | 5 |
| A-41 | B-3 | C-2 | 80 | 15 | 5 |
| A-42 | B-3 | C-2 | 80 | 15 | 5 |
| A-43 | B-3 | C-2 | 80 | 15 | 5 |
| A-44 | B-3 | C-2 | 80 | 15 | 5 |
| A-45 | B-3 | C-2 | 80 | 15 | 5 |
| A-46 | B-3 | C-2 | 80 | 15 | 5 |
| A-47 | B-3 | C-2 | 80 | 15 | 5 |
| A-49 | B-3 | C-2 | 80 | 15 | 5 |
| A-50 | B-3 | C-2 | 80 | 15 | 5 |
| A-51 | B-3 | C-2 | 80 | 15 | 5 |
| A-52 | B-3 | C-2 | 80 | 15 | 5 |
| A-53 | B-3 | C-2 | 80 | 15 | 5 |
| A-54 | B-3 | C-2 | 80 | 15 | 5 |
| A-55 | B-3 | C-2 | 80 | 15 | 5 |
| A-56 | B-3 | C-2 | 80 | 15 | 5 |
| A-57 | B-3 | C-2 | 80 | 15 | 5 |
| A-58 | B-3 | C-2 | 80 | 15 | 5 |
| A-59 | B-3 | C-2 | 80 | 15 | 5 |
| A-60 | B-3 | C-2 | 80 | 15 | 5 |

TABLE 38

| | | | Ratio of | | |
|---|---|---|---|---|---|
| | | | units | | |
| | | | (mol %) | | |
| A | B | C | A | B | C |
| A-61 | B-3 | C-2 | 80 | 15 | 5 |
| A-62 | B-3 | C-2 | 80 | 15 | 5 |
| A-63 | B-3 | C-2 | 80 | 15 | 5 |
| A-64 | B-3 | C-2 | 80 | 15 | 5 |
| A-65 | B-3 | C-2 | 80 | 15 | 5 |
| A-66 | B-3 | C-2 | 80 | 15 | 5 |
| A-67 | B-3 | C-2 | 80 | 15 | 5 |
| A-68 | B-3 | C-2 | 80 | 15 | 5 |
| A-69 | B-3 | C-2 | 80 | 15 | 5 |
| A-70 | B-3 | C-2 | 80 | 15 | 5 |
| A-71 | B-3 | C-2 | 80 | 15 | 5 |
| A-72 | B-3 | C-2 | 80 | 15 | 5 |
| A-73 | B-3 | C-2 | 80 | 15 | 5 |
| A-74 | B-3 | C-2 | 80 | 15 | 5 |
| A-75 | B-3 | C-2 | 80 | 15 | 5 |
| A-76 | B-3 | C-2 | 80 | 15 | 5 |
| A-77 | B-3 | C-2 | 80 | 15 | 5 |
| A-78 | B-3 | C-2 | 80 | 15 | 5 |
| A-79 | B-3 | C-2 | 80 | 15 | 5 |
| A-80 | B-3 | C-2 | 80 | 15 | 5 |
| A-81 | B-3 | C-2 | 80 | 15 | 5 |
| A-82 | B-3 | C-2 | 80 | 15 | 5 |
| A-83 | B-3 | C-2 | 80 | 15 | 5 |
| A-84 | B-3 | C-2 | 80 | 15 | 5 |
| A-85 | B-3 | C-2 | 80 | 15 | 5 |
| A-86 | B-3 | C-2 | 80 | 15 | 5 |
| A-87 | B-3 | C-2 | 80 | 15 | 5 |
| A-88 | B-3 | C-2 | 80 | 15 | 5 |
| A-89 | B-3 | C-2 | 80 | 15 | 5 |
| A-90 | B-3 | C-2 | 80 | 15 | 5 |
| A-91 | B-3 | C-2 | 80 | 15 | 5 |
| A-92 | B-3 | C-2 | 80 | 15 | 5 |
| A-93 | B-3 | C-2 | 80 | 15 | 5 |
| A-94 | B-3 | C-2 | 80 | 15 | 5 |
| A-95 | B-3 | C-2 | 80 | 15 | 5 |
| A-96 | B-3 | C-2 | 80 | 15 | 5 |
| A-100 | B-3 | C-2 | 80 | 15 | 5 |
| A-101 | B-3 | C-2 | 80 | 15 | 5 |
| A-102 | B-3 | C-2 | 80 | 15 | 5 |
| A-103 | B-3 | C-2 | 80 | 15 | 5 |
| A-104 | B-3 | C-2 | 80 | 15 | 5 |
| A-105 | B-3 | C-2 | 80 | 15 | 5 |
| A-106 | B-3 | C-2 | 80 | 15 | 5 |
| A-107 | B-3 | C-2 | 80 | 15 | 5 |
| A-108 | B-3 | C-2 | 80 | 15 | 5 |
| A-109 | B-3 | C-2 | 80 | 15 | 5 |
| A-110 | B-3 | C-2 | 80 | 15 | 5 |
| A-111 | B-3 | C-2 | 80 | 15 | 5 |
| A-112 | B-3 | C-2 | 80 | 15 | 5 |
| A-113 | B-3 | C-2 | 80 | 15 | 5 |
| A-114 | B-3 | C-2 | 80 | 15 | 5 |
| A-115 | B-3 | C-2 | 80 | 15 | 5 |
| A-116 | B-3 | C-2 | 80 | 15 | 5 |
| A-117 | B-3 | C-2 | 80 | 15 | 5 |
| A-118 | B-3 | C-2 | 80 | 15 | 5 |
| A-119 | B-3 | C-2 | 80 | 15 | 5 |
| A-120 | B-3 | C-2 | 80 | 15 | 5 |
| A-121 | B-3 | C-2 | 80 | 15 | 5 |
| A-122 | B-3 | C-2 | 80 | 15 | 5 |
| A-123 | B-3 | C-2 | 80 | 15 | 5 |

TABLE 39

| | | | Ratio of | | |
|---|---|---|---|---|---|
| | | | units | | |
| | | | (mol %) | | |
| A | B | C | A | B | C |
| A-124 | B-3 | C-2 | 80 | 15 | 5 |
| A-125 | B-3 | C-2 | 80 | 15 | 5 |
| A-126 | B-3 | C-2 | 80 | 15 | 5 |
| A-127 | B-3 | C-2 | 80 | 15 | 5 |
| A-128 | B-3 | C-2 | 80 | 15 | 5 |
| A-129 | B-3 | C-2 | 80 | 15 | 5 |
| A-130 | B-3 | C-2 | 80 | 15 | 5 |
| A-131 | B-3 | C-2 | 80 | 15 | 5 |
| A-132 | B-3 | C-2 | 80 | 15 | 5 |
| A-133 | B-3 | C-2 | 80 | 15 | 5 |
| A-134 | B-3 | C-2 | 80 | 15 | 5 |
| A-135 | B-3 | C-2 | 80 | 15 | 5 |
| A-136 | B-3 | C-2 | 80 | 15 | 5 |
| A-137 | B-3 | C-2 | 80 | 15 | 5 |
| A-138 | B-3 | C-2 | 80 | 15 | 5 |
| A-139 | B-3 | C-2 | 80 | 15 | 5 |
| A-140 | B-3 | C-2 | 80 | 15 | 5 |
| A-141 | B-3 | C-2 | 80 | 15 | 5 |
| A-142 | B-3 | C-2 | 80 | 15 | 5 |
| A-143 | B-3 | C-2 | 80 | 15 | 5 |
| A-144 | B-3 | C-2 | 80 | 15 | 5 |
| A-145 | B-3 | C-2 | 80 | 15 | 5 |
| A-146 | B-3 | C-2 | 80 | 15 | 5 |
| A-147 | B-3 | C-2 | 80 | 15 | 5 |
| A-160 | B-3 | C-2 | 80 | 15 | 5 |
| A-161 | B-3 | C-2 | 80 | 15 | 5 |
| A-162 | B-3 | C-2 | 80 | 15 | 5 |
| A-163 | B-3 | C-2 | 80 | 15 | 5 |
| A-164 | B-3 | C-2 | 80 | 15 | 5 |
| A-165 | B-3 | C-2 | 80 | 15 | 5 |
| A-166 | B-3 | C-2 | 80 | 15 | 5 |
| A-167 | B-3 | C-2 | 80 | 15 | 5 |
| A-168 | B-3 | C-2 | 80 | 15 | 5 |
| A-169 | B-3 | C-2 | 80 | 15 | 5 |
| A-170 | B-3 | C-2 | 80 | 15 | 5 |
| A-171 | B-3 | C-2 | 80 | 15 | 5 |
| A-172 | B-3 | C-2 | 80 | 15 | 5 |
| A-173 | B-3 | C-2 | 80 | 15 | 5 |
| A-174 | B-3 | C-2 | 80 | 15 | 5 |
| A-175 | B-3 | C-2 | 80 | 15 | 5 |
| A-176 | B-3 | C-2 | 80 | 15 | 5 |
| A-177 | B-3 | C-2 | 80 | 15 | 5 |
| A-178 | B-3 | C-2 | 80 | 15 | 5 |
| A-179 | B-3 | C-2 | 80 | 15 | 5 |
| A-180 | B-3 | C-2 | 80 | 15 | 5 |
| A-181 | B-3 | C-2 | 80 | 15 | 5 |
| A-182 | B-3 | C-2 | 80 | 15 | 5 |
| A-183 | B-3 | C-2 | 80 | 15 | 5 |
| A-190 | B-3 | C-2 | 80 | 15 | 5 |
| A-191 | B-3 | C-2 | 80 | 15 | 5 |
| A-192 | B-3 | C-2 | 80 | 15 | 5 |
| A-193 | B-3 | C-2 | 80 | 15 | 5 |
| A-194 | B-3 | C-2 | 80 | 15 | 5 |
| A-195 | B-3 | C-2 | 80 | 15 | 5 |
| A-196 | B-3 | C-2 | 80 | 15 | 5 |
| A-197 | B-3 | C-2 | 80 | 15 | 5 |
| A-198 | B-3 | C-2 | 80 | 15 | 5 |
| A-199 | B-3 | C-2 | 80 | 15 | 5 |

TABLE 40

| | | | Ratio of | | |
|---|---|---|---|---|---|
| | | | units | | |
| | | | (mol %) | | |
| A | B | C | A | B | C |
| A-200 | B-3 | C-2 | 80 | 15 | 5 |
| A-201 | B-3 | C-2 | 80 | 15 | 5 |
| A-202 | B-3 | C-2 | 80 | 15 | 5 |
| A-203 | B-3 | C-2 | 80 | 15 | 5 |
| A-204 | B-3 | C-2 | 80 | 15 | 5 |
| A-205 | B-3 | C-2 | 80 | 15 | 5 |
| A-206 | B-3 | C-2 | 80 | 15 | 5 |
| A-207 | B-3 | C-2 | 80 | 15 | 5 |
| A-208 | B-3 | C-2 | 80 | 15 | 5 |
| A-209 | B-3 | C-2 | 80 | 15 | 5 |
| A-210 | B-3 | C-2 | 80 | 15 | 5 |
| A-211 | B-3 | C-2 | 80 | 15 | 5 |
| A-212 | B-3 | C-2 | 80 | 15 | 5 |
| A-213 | B-3 | C-2 | 80 | 15 | 5 |
| A-220 | B-3 | C-2 | 80 | 15 | 5 |
| A-221 | B-3 | C-2 | 80 | 15 | 5 |
| A-222 | B-3 | C-2 | 80 | 15 | 5 |
| A-223 | B-3 | C-2 | 80 | 15 | 5 |
| A-224 | B-3 | C-2 | 80 | 15 | 5 |
| A-225 | B-3 | C-2 | 80 | 15 | 5 |
| A-226 | B-3 | C-2 | 80 | 15 | 5 |
| A-227 | B-3 | C-2 | 80 | 15 | 5 |
| A-228 | B-3 | C-2 | 80 | 15 | 5 |
| A-229 | B-3 | C-2 | 80 | 15 | 5 |
| A-230 | B-3 | C-2 | 80 | 15 | 5 |
| A-231 | B-3 | C-2 | 80 | 15 | 5 |
| A-232 | B-3 | C-2 | 80 | 15 | 5 |
| A-233 | B-3 | C-2 | 80 | 15 | 5 |
| A-234 | B-3 | C-2 | 80 | 15 | 5 |
| A-235 | B-3 | C-2 | 80 | 15 | 5 |
| A-236 | B-3 | C-2 | 80 | 15 | 5 |
| A-237 | B-3 | C-2 | 80 | 15 | 5 |
| A-238 | B-3 | C-2 | 80 | 15 | 5 |
| A-239 | B-3 | C-2 | 80 | 15 | 5 |
| A-240 | B-3 | C-2 | 80 | 15 | 5 |

TABLE 41

| | | | Ratio of | | |
|---|---|---|---|---|---|
| | | | units | | |
| | | | (mol %) | | |
| A | B | C | A | B | C |
| A-1 | B-3 | C-3 | 80 | 15 | 5 |
| A-2 | B-3 | C-3 | 80 | 15 | 5 |
| A-3 | B-3 | C-3 | 80 | 15 | 5 |
| A-4 | B-3 | C-3 | 80 | 15 | 5 |
| A-5 | B-3 | C-3 | 80 | 15 | 5 |
| A-6 | B-3 | C-3 | 80 | 15 | 5 |
| A-7 | B-3 | C-3 | 80 | 15 | 5 |
| A-8 | B-3 | C-3 | 80 | 15 | 5 |
| A-9 | B-3 | C-3 | 80 | 15 | 5 |
| A-10 | B-3 | C-3 | 80 | 15 | 5 |
| A-11 | B-3 | C-3 | 80 | 15 | 5 |
| A-12 | B-3 | C-3 | 80 | 15 | 5 |
| A-13 | B-3 | C-3 | 80 | 15 | 5 |
| A-14 | B-3 | C-3 | 80 | 15 | 5 |
| A-15 | B-3 | C-3 | 80 | 15 | 5 |
| A-16 | B-3 | C-3 | 80 | 15 | 5 |
| A-17 | B-3 | C-3 | 80 | 15 | 5 |
| A-18 | B-3 | C-3 | 80 | 15 | 5 |
| A-19 | B-3 | C-3 | 80 | 15 | 5 |
| A-20 | B-3 | C-3 | 80 | 15 | 5 |
| A-21 | B-3 | C-3 | 80 | 15 | 5 |
| A-22 | B-3 | C-3 | 80 | 15 | 5 |
| A-23 | B-3 | C-3 | 80 | 15 | 5 |
| A-24 | B-3 | C-3 | 80 | 15 | 5 |
| A-25 | B-3 | C-3 | 80 | 15 | 5 |
| A-26 | B-3 | C-3 | 80 | 15 | 5 |
| A-28 | B-3 | C-3 | 80 | 15 | 5 |
| A-29 | B-3 | C-3 | 80 | 15 | 5 |
| A-30 | B-3 | C-3 | 80 | 15 | 5 |
| A-31 | B-3 | C-3 | 80 | 15 | 5 |
| A-32 | B-3 | C-3 | 80 | 15 | 5 |
| A-33 | B-3 | C-3 | 80 | 15 | 5 |
| A-34 | B-3 | C-3 | 80 | 15 | 5 |
| A-35 | B-3 | C-3 | 80 | 15 | 5 |
| A-36 | B-3 | C-3 | 80 | 15 | 5 |
| A-37 | B-3 | C-3 | 80 | 15 | 5 |
| A-38 | B-3 | C-3 | 80 | 15 | 5 |
| A-39 | B-3 | C-3 | 80 | 15 | 5 |
| A-40 | B-3 | C-3 | 80 | 15 | 5 |
| A-41 | B-3 | C-3 | 80 | 15 | 5 |
| A-42 | B-3 | C-3 | 80 | 15 | 5 |
| A-43 | B-3 | C-3 | 80 | 15 | 5 |
| A-44 | B-3 | C-3 | 80 | 15 | 5 |
| A-45 | B-3 | C-3 | 80 | 15 | 5 |
| A-46 | B-3 | C-3 | 80 | 15 | 5 |
| A-47 | B-3 | C-3 | 80 | 15 | 5 |
| A-49 | B-3 | C-3 | 80 | 15 | 5 |
| A-50 | B-3 | C-3 | 80 | 15 | 5 |
| A-51 | B-3 | C-3 | 80 | 15 | 5 |
| A-52 | B-3 | C-3 | 80 | 15 | 5 |
| A-53 | B-3 | C-3 | 80 | 15 | 5 |
| A-54 | B-3 | C-3 | 80 | 15 | 5 |
| A-55 | B-3 | C-3 | 80 | 15 | 5 |
| A-56 | B-3 | C-3 | 80 | 15 | 5 |
| A-57 | B-3 | C-3 | 80 | 15 | 5 |
| A-58 | B-3 | C-3 | 80 | 15 | 5 |
| A-59 | B-3 | C-3 | 80 | 15 | 5 |
| A-60 | B-3 | C-3 | 80 | 15 | 5 |

TABLE 42

| | | | Ratio of | | |
|---|---|---|---|---|---|
| | | | units | | |
| | | | (mol %) | | |
| A | B | C | A | B | C |
| A-61 | B-3 | C-3 | 80 | 15 | 5 |
| A-62 | B-3 | C-3 | 80 | 15 | 5 |
| A-63 | B-3 | C-3 | 80 | 15 | 5 |
| A-64 | B-3 | C-3 | 80 | 15 | 5 |
| A-65 | B-3 | C-3 | 80 | 15 | 5 |
| A-66 | B-3 | C-3 | 80 | 15 | 5 |
| A-67 | B-3 | C-3 | 80 | 15 | 5 |
| A-68 | B-3 | C-3 | 80 | 15 | 5 |
| A-69 | B-3 | C-3 | 80 | 15 | 5 |
| A-70 | B-3 | C-3 | 80 | 15 | 5 |
| A-71 | B-3 | C-3 | 80 | 15 | 5 |
| A-72 | B-3 | C-3 | 80 | 15 | 5 |
| A-73 | B-3 | C-3 | 80 | 15 | 5 |
| A-74 | B-3 | C-3 | 80 | 15 | 5 |
| A-75 | B-3 | C-3 | 80 | 15 | 5 |
| A-76 | B-3 | C-3 | 80 | 15 | 5 |
| A-77 | B-3 | C-3 | 80 | 15 | 5 |
| A-78 | B-3 | C-3 | 80 | 15 | 5 |
| A-79 | B-3 | C-3 | 80 | 15 | 5 |
| A-80 | B-3 | C-3 | 80 | 15 | 5 |
| A-81 | B-3 | C-3 | 80 | 15 | 5 |
| A-82 | B-3 | C-3 | 80 | 15 | 5 |
| A-83 | B-3 | C-3 | 80 | 15 | 5 |
| A-84 | B-3 | C-3 | 80 | 15 | 5 |
| A-85 | B-3 | C-3 | 80 | 15 | 5 |
| A-86 | B-3 | C-3 | 80 | 15 | 5 |
| A-87 | B-3 | C-3 | 80 | 15 | 5 |
| A-88 | B-3 | C-3 | 80 | 15 | 5 |
| A-89 | B-3 | C-3 | 80 | 15 | 5 |
| A-90 | B-3 | C-3 | 80 | 15 | 5 |
| A-91 | B-3 | C-3 | 80 | 15 | 5 |
| A-92 | B-3 | C-3 | 80 | 15 | 5 |
| A-93 | B-3 | C-3 | 80 | 15 | 5 |
| A-94 | B-3 | C-3 | 80 | 15 | 5 |
| A-95 | B-3 | C-3 | 80 | 15 | 5 |
| A-96 | B-3 | C-3 | 80 | 15 | 5 |
| A-100 | B-3 | C-3 | 80 | 15 | 5 |
| A-101 | B-3 | C-3 | 80 | 15 | 5 |
| A-102 | B-3 | C-3 | 80 | 15 | 5 |
| A-103 | B-3 | C-3 | 80 | 15 | 5 |
| A-104 | B-3 | C-3 | 80 | 15 | 5 |
| A-105 | B-3 | C-3 | 80 | 15 | 5 |
| A-106 | B-3 | C-3 | 80 | 15 | 5 |
| A-107 | B-3 | C-3 | 80 | 15 | 5 |
| A-108 | B-3 | C-3 | 80 | 15 | 5 |
| A-109 | B-3 | C-3 | 80 | 15 | 5 |
| A-110 | B-3 | C-3 | 80 | 15 | 5 |
| A-111 | B-3 | C-3 | 80 | 15 | 5 |
| A-112 | B-3 | C-3 | 80 | 15 | 5 |
| A-113 | B-3 | C-3 | 80 | 15 | 5 |
| A-114 | B-3 | C-3 | 80 | 15 | 5 |
| A-115 | B-3 | C-3 | 80 | 15 | 5 |
| A-116 | B-3 | C-3 | 80 | 15 | 5 |
| A-117 | B-3 | C-3 | 80 | 15 | 5 |
| A-118 | B-3 | C-3 | 80 | 15 | 5 |
| A-119 | B-3 | C-3 | 80 | 15 | 5 |
| A-120 | B-3 | C-3 | 80 | 15 | 5 |
| A-121 | B-3 | C-3 | 80 | 15 | 5 |
| A-122 | B-3 | C-3 | 80 | 15 | 5 |
| A-123 | B-3 | C-3 | 80 | 15 | 5 |

TABLE 43

| | | | Ratio of | | |
|---|---|---|---|---|---|
| | | | units | | |
| | | | (mol %) | | |
| A | B | C | A | B | C |
| A-124 | B-3 | C-3 | 80 | 15 | 5 |
| A-125 | B-3 | C-3 | 80 | 15 | 5 |
| A-126 | B-3 | C-3 | 80 | 15 | 5 |
| A-127 | B-3 | C-3 | 80 | 15 | 5 |
| A-128 | B-3 | C-3 | 80 | 15 | 5 |
| A-129 | B-3 | C-3 | 80 | 15 | 5 |
| A-130 | B-3 | C-3 | 80 | 15 | 5 |
| A-131 | B-3 | C-3 | 80 | 15 | 5 |
| A-132 | B-3 | C-3 | 80 | 15 | 5 |
| A-133 | B-3 | C-3 | 80 | 15 | 5 |
| A-134 | B-3 | C-3 | 80 | 15 | 5 |
| A-135 | B-3 | C-3 | 80 | 15 | 5 |
| A-136 | B-3 | C-3 | 80 | 15 | 5 |
| A-137 | B-3 | C-3 | 80 | 15 | 5 |
| A-138 | B-3 | C-3 | 80 | 15 | 5 |
| A-139 | B-3 | C-3 | 80 | 15 | 5 |
| A-140 | B-3 | C-3 | 80 | 15 | 5 |
| A-141 | B-3 | C-3 | 80 | 15 | 5 |
| A-142 | B-3 | C-3 | 80 | 15 | 5 |
| A-143 | B-3 | C-3 | 80 | 15 | 5 |
| A-144 | B-3 | C-3 | 80 | 15 | 5 |
| A-145 | B-3 | C-3 | 80 | 15 | 5 |
| A-146 | B-3 | C-3 | 80 | 15 | 5 |
| A-147 | B-3 | C-3 | 80 | 15 | 5 |
| A-160 | B-3 | C-3 | 80 | 15 | 5 |
| A-161 | B-3 | C-3 | 80 | 15 | 5 |
| A-162 | B-3 | C-3 | 80 | 15 | 5 |
| A-163 | B-3 | C-3 | 80 | 15 | 5 |
| A-164 | B-3 | C-3 | 80 | 15 | 5 |
| A-165 | B-3 | C-3 | 80 | 15 | 5 |
| A-166 | B-3 | C-3 | 80 | 15 | 5 |
| A-167 | B-3 | C-3 | 80 | 15 | 5 |
| A-168 | B-3 | C-3 | 80 | 15 | 5 |
| A-169 | B-3 | C-3 | 80 | 15 | 5 |
| A-170 | B-3 | C-3 | 80 | 15 | 5 |
| A-171 | B-3 | C-3 | 80 | 15 | 5 |
| A-172 | B-3 | C-3 | 80 | 15 | 5 |
| A-173 | B-3 | C-3 | 80 | 15 | 5 |
| A-174 | B-3 | C-3 | 80 | 15 | 5 |
| A-175 | B-3 | C-3 | 80 | 15 | 5 |
| A-176 | B-3 | C-3 | 80 | 15 | 5 |
| A-177 | B-3 | C-3 | 80 | 15 | 5 |
| A-178 | B-3 | C-3 | 80 | 15 | 5 |
| A-179 | B-3 | C-3 | 80 | 15 | 5 |
| A-180 | B-3 | C-3 | 80 | 15 | 5 |
| A-181 | B-3 | C-3 | 80 | 15 | 5 |
| A-182 | B-3 | C-3 | 80 | 15 | 5 |
| A-183 | B-3 | C-3 | 80 | 15 | 5 |
| A-190 | B-3 | C-3 | 80 | 15 | 5 |
| A-191 | B-3 | C-3 | 80 | 15 | 5 |
| A-192 | B-3 | C-3 | 80 | 15 | 5 |
| A-193 | B-3 | C-3 | 80 | 15 | 5 |
| A-194 | B-3 | C-3 | 80 | 15 | 5 |
| A-195 | B-3 | C-3 | 80 | 15 | 5 |
| A-196 | B-3 | C-3 | 80 | 15 | 5 |
| A-197 | B-3 | C-3 | 80 | 15 | 5 |
| A-198 | B-3 | C-3 | 80 | 15 | 5 |
| A-199 | B-3 | C-3 | 80 | 15 | 5 |

TABLE 44

| | | | Ratio of | | |
|---|---|---|---|---|---|
| | | | units | | |
| | | | (mol %) | | |
| A | B | C | A | B | C |
| A-200 | B-3 | C-3 | 80 | 15 | 5 |
| A-201 | B-3 | C-3 | 80 | 15 | 5 |
| A-202 | B-3 | C-3 | 80 | 15 | 5 |
| A-203 | B-3 | C-3 | 80 | 15 | 5 |
| A-204 | B-3 | C-3 | 80 | 15 | 5 |
| A-205 | B-3 | C-3 | 80 | 15 | 5 |
| A-206 | B-3 | C-3 | 80 | 15 | 5 |
| A-207 | B-3 | C-3 | 80 | 15 | 5 |
| A-208 | B-3 | C-3 | 80 | 15 | 5 |
| A-209 | B-3 | C-3 | 80 | 15 | 5 |
| A-210 | B-3 | C-3 | 80 | 15 | 5 |
| A-211 | B-3 | C-3 | 80 | 15 | 5 |
| A-212 | B-3 | C-3 | 80 | 15 | 5 |
| A-213 | B-3 | C-3 | 80 | 15 | 5 |
| A-220 | B-3 | C-3 | 80 | 15 | 5 |
| A-221 | B-3 | C-3 | 80 | 15 | 5 |
| A-222 | B-3 | C-3 | 80 | 15 | 5 |
| A-223 | B-3 | C-3 | 80 | 15 | 5 |
| A-224 | B-3 | C-3 | 80 | 15 | 5 |
| A-225 | B-3 | C-3 | 80 | 15 | 5 |
| A-226 | B-3 | C-3 | 80 | 15 | 5 |
| A-227 | B-3 | C-3 | 80 | 15 | 5 |
| A-228 | B-3 | C-3 | 80 | 15 | 5 |
| A-229 | B-3 | C-3 | 80 | 15 | 5 |
| A-230 | B-3 | C-3 | 80 | 15 | 5 |
| A-231 | B-3 | C-3 | 80 | 15 | 5 |
| A-232 | B-3 | C-3 | 80 | 15 | 5 |
| A-233 | B-3 | C-3 | 80 | 15 | 5 |
| A-234 | B-3 | C-3 | 80 | 15 | 5 |
| A-235 | B-3 | C-3 | 80 | 15 | 5 |
| A-236 | B-3 | C-3 | 80 | 15 | 5 |
| A-237 | B-3 | C-3 | 80 | 15 | 5 |
| A-238 | B-3 | C-3 | 80 | 15 | 5 |
| A-239 | B-3 | C-3 | 80 | 15 | 5 |
| A-240 | B-3 | C-3 | 80 | 15 | 5 |

TABLE 45

| | | | Ratio of | | |
|---|---|---|---|---|---|
| | | | units | | |
| | | | (mol %) | | |
| A | B | C | A | B | C |
| A-1 | B-3 | C-4 | 80 | 15 | 5 |
| A-2 | B-3 | C-4 | 80 | 15 | 5 |
| A-3 | B-3 | C-4 | 80 | 15 | 5 |
| A-4 | B-3 | C-4 | 80 | 15 | 5 |
| A-5 | B-3 | C-4 | 80 | 15 | 5 |
| A-6 | B-3 | C-4 | 80 | 15 | 5 |
| A-7 | B-3 | C-4 | 80 | 15 | 5 |
| A-8 | B-3 | C-4 | 80 | 15 | 5 |
| A-9 | B-3 | C-4 | 80 | 15 | 5 |
| A-10 | B-3 | C-4 | 80 | 15 | 5 |
| A-11 | B-3 | C-4 | 80 | 15 | 5 |
| A-12 | B-3 | C-4 | 80 | 15 | 5 |
| A-13 | B-3 | C-4 | 80 | 15 | 5 |
| A-14 | B-3 | C-4 | 80 | 15 | 5 |
| A-15 | B-3 | C-4 | 80 | 15 | 5 |
| A-16 | B-3 | C-4 | 80 | 15 | 5 |
| A-17 | B-3 | C-4 | 80 | 15 | 5 |
| A-18 | B-3 | C-4 | 80 | 15 | 5 |
| A-19 | B-3 | C-4 | 80 | 15 | 5 |
| A-20 | B-3 | C-4 | 80 | 15 | 5 |
| A-21 | B-3 | C-4 | 80 | 15 | 5 |
| A-22 | B-3 | C-4 | 80 | 15 | 5 |
| A-23 | B-3 | C-4 | 80 | 15 | 5 |
| A-24 | B-3 | C-4 | 80 | 15 | 5 |
| A-25 | B-3 | C-4 | 80 | 15 | 5 |
| A-26 | B-3 | C-4 | 80 | 15 | 5 |
| A-28 | B-3 | C-4 | 80 | 15 | 5 |
| A-29 | B-3 | C-4 | 80 | 15 | 5 |
| A-30 | B-3 | C-4 | 80 | 15 | 5 |
| A-31 | B-3 | C-4 | 80 | 15 | 5 |
| A-32 | B-3 | C-4 | 80 | 15 | 5 |
| A-33 | B-3 | C-4 | 80 | 15 | 5 |
| A-34 | B-3 | C-4 | 80 | 15 | 5 |
| A-35 | B-3 | C-4 | 80 | 15 | 5 |
| A-36 | B-3 | C-4 | 80 | 15 | 5 |
| A-37 | B-3 | C-4 | 80 | 15 | 5 |
| A-38 | B-3 | C-4 | 80 | 15 | 5 |
| A-39 | B-3 | C-4 | 80 | 15 | 5 |
| A-40 | B-3 | C-4 | 80 | 15 | 5 |
| A-41 | B-3 | C-4 | 80 | 15 | 5 |
| A-42 | B-3 | C-4 | 80 | 15 | 5 |
| A-43 | B-3 | C-4 | 80 | 15 | 5 |
| A-44 | B-3 | C-4 | 80 | 15 | 5 |
| A-45 | B-3 | C-4 | 80 | 15 | 5 |
| A-46 | B-3 | C-4 | 80 | 15 | 5 |
| A-47 | B-3 | C-4 | 80 | 15 | 5 |
| A-49 | B-3 | C-4 | 80 | 15 | 5 |
| A-50 | B-3 | C-4 | 80 | 15 | 5 |
| A-51 | B-3 | C-4 | 80 | 15 | 5 |
| A-52 | B-3 | C-4 | 80 | 15 | 5 |
| A-53 | B-3 | C-4 | 80 | 15 | 5 |
| A-54 | B-3 | C-4 | 80 | 15 | 5 |
| A-55 | B-3 | C-4 | 80 | 15 | 5 |
| A-56 | B-3 | C-4 | 80 | 15 | 5 |
| A-57 | B-3 | C-4 | 80 | 15 | 5 |
| A-58 | B-3 | C-4 | 80 | 15 | 5 |
| A-59 | B-3 | C-4 | 80 | 15 | 5 |
| A-60 | B-3 | C-4 | 80 | 15 | 5 |

TABLE 46

| | | | Ratio of | | |
|---|---|---|---|---|---|
| | | | units | | |
| | | | (mol %) | | |
| A | B | C | A | B | C |
| A-61 | B-3 | C-4 | 80 | 15 | 5 |
| A-62 | B-3 | C-4 | 80 | 15 | 5 |
| A-63 | B-3 | C-4 | 80 | 15 | 5 |
| A-64 | B-3 | C-4 | 80 | 15 | 5 |
| A-65 | B-3 | C-4 | 80 | 15 | 5 |
| A-66 | B-3 | C-4 | 80 | 15 | 5 |
| A-67 | B-3 | C-4 | 80 | 15 | 5 |
| A-68 | B-3 | C-4 | 80 | 15 | 5 |
| A-69 | B-3 | C-4 | 80 | 15 | 5 |
| A-70 | B-3 | C-4 | 80 | 15 | 5 |
| A-71 | B-3 | C-4 | 80 | 15 | 5 |
| A-72 | B-3 | C-4 | 80 | 15 | 5 |
| A-73 | B-3 | C-4 | 80 | 15 | 5 |
| A-74 | B-3 | C-4 | 80 | 15 | 5 |
| A-75 | B-3 | C-4 | 80 | 15 | 5 |
| A-76 | B-3 | C-4 | 80 | 15 | 5 |
| A-77 | B-3 | C-4 | 80 | 15 | 5 |
| A-78 | B-3 | C-4 | 80 | 15 | 5 |
| A-79 | B-3 | C-4 | 80 | 15 | 5 |
| A-80 | B-3 | C-4 | 80 | 15 | 5 |
| A-81 | B-3 | C-4 | 80 | 15 | 5 |
| A-82 | B-3 | C-4 | 80 | 15 | 5 |
| A-83 | B-3 | C-4 | 80 | 15 | 5 |
| A-84 | B-3 | C-4 | 80 | 15 | 5 |
| A-85 | B-3 | C-4 | 80 | 15 | 5 |
| A-86 | B-3 | C-4 | 80 | 15 | 5 |
| A-87 | B-3 | C-4 | 80 | 15 | 5 |
| A-88 | B-3 | C-4 | 80 | 15 | 5 |
| A-89 | B-3 | C-4 | 80 | 15 | 5 |
| A-90 | B-3 | C-4 | 80 | 15 | 5 |
| A-91 | B-3 | C-4 | 80 | 15 | 5 |
| A-92 | B-3 | C-4 | 80 | 15 | 5 |
| A-93 | B-3 | C-4 | 80 | 15 | 5 |
| A-94 | B-3 | C-4 | 80 | 15 | 5 |
| A-95 | B-3 | C-4 | 80 | 15 | 5 |
| A-96 | B-3 | C-4 | 80 | 15 | 5 |
| A-100 | B-3 | C-4 | 80 | 15 | 5 |
| A-101 | B-3 | C-4 | 80 | 15 | 5 |
| A-102 | B-3 | C-4 | 80 | 15 | 5 |
| A-103 | B-3 | C-4 | 80 | 15 | 5 |
| A-104 | B-3 | C-4 | 80 | 15 | 5 |
| A-105 | B-3 | C-4 | 80 | 15 | 5 |
| A-106 | B-3 | C-4 | 80 | 15 | 5 |
| A-107 | B-3 | C-4 | 80 | 15 | 5 |
| A-108 | B-3 | C-4 | 80 | 15 | 5 |
| A-109 | B-3 | C-4 | 80 | 15 | 5 |
| A-110 | B-3 | C-4 | 80 | 15 | 5 |
| A-111 | B-3 | C-4 | 80 | 15 | 5 |
| A-112 | B-3 | C-4 | 80 | 15 | 5 |
| A-113 | B-3 | C-4 | 80 | 15 | 5 |
| A-114 | B-3 | C-4 | 80 | 15 | 5 |
| A-115 | B-3 | C-4 | 80 | 15 | 5 |
| A-116 | B-3 | C-4 | 80 | 15 | 5 |
| A-117 | B-3 | C-4 | 80 | 15 | 5 |
| A-118 | B-3 | C-4 | 80 | 15 | 5 |
| A-119 | B-3 | C-4 | 80 | 15 | 5 |
| A-120 | B-3 | C-4 | 80 | 15 | 5 |
| A-121 | B-3 | C-4 | 80 | 15 | 5 |
| A-122 | B-3 | C-4 | 80 | 15 | 5 |
| A-123 | B-3 | C-4 | 80 | 15 | 5 |

TABLE 47

| | | | Ratio of | | |
|---|---|---|---|---|---|
| | | | units | | |
| | | | (mol %) | | |
| A | B | C | A | B | C |
| A-124 | B-3 | C-4 | 80 | 15 | 5 |
| A-125 | B-3 | C-4 | 80 | 15 | 5 |
| A-126 | B-3 | C-4 | 80 | 15 | 5 |
| A-127 | B-3 | C-4 | 80 | 15 | 5 |
| A-128 | B-3 | C-4 | 80 | 15 | 5 |
| A-129 | B-3 | C-4 | 80 | 15 | 5 |
| A-130 | B-3 | C-4 | 80 | 15 | 5 |
| A-131 | B-3 | C-4 | 80 | 15 | 5 |
| A-132 | B-3 | C-4 | 80 | 15 | 5 |
| A-133 | B-3 | C-4 | 80 | 15 | 5 |
| A-134 | B-3 | C-4 | 80 | 15 | 5 |
| A-135 | B-3 | C-4 | 80 | 15 | 5 |
| A-136 | B-3 | C-4 | 80 | 15 | 5 |
| A-137 | B-3 | C-4 | 80 | 15 | 5 |
| A-138 | B-3 | C-4 | 80 | 15 | 5 |
| A-139 | B-3 | C-4 | 80 | 15 | 5 |
| A-140 | B-3 | C-4 | 80 | 15 | 5 |
| A-141 | B-3 | C-4 | 80 | 15 | 5 |
| A-142 | B-3 | C-4 | 80 | 15 | 5 |
| A-143 | B-3 | C-4 | 80 | 15 | 5 |
| A-144 | B-3 | C-4 | 80 | 15 | 5 |
| A-145 | B-3 | C-4 | 80 | 15 | 5 |
| A-146 | B-3 | C-4 | 80 | 15 | 5 |
| A-147 | B-3 | C-4 | 80 | 15 | 5 |
| A-160 | B-3 | C-4 | 80 | 15 | 5 |
| A-161 | B-3 | C-4 | 80 | 15 | 5 |
| A-162 | B-3 | C-4 | 80 | 15 | 5 |
| A-163 | B-3 | C-4 | 80 | 15 | 5 |
| A-164 | B-3 | C-4 | 80 | 15 | 5 |
| A-165 | B-3 | C-4 | 80 | 15 | 5 |
| A-166 | B-3 | C-4 | 80 | 15 | 5 |
| A-167 | B-3 | C-4 | 80 | 15 | 5 |
| A-168 | B-3 | C-4 | 80 | 15 | 5 |
| A-169 | B-3 | C-4 | 80 | 15 | 5 |
| A-170 | B-3 | C-4 | 80 | 15 | 5 |
| A-171 | B-3 | C-4 | 80 | 15 | 5 |
| A-172 | B-3 | C-4 | 80 | 15 | 5 |
| A-173 | B-3 | C-4 | 80 | 15 | 5 |
| A-174 | B-3 | C-4 | 80 | 15 | 5 |
| A-175 | B-3 | C-4 | 80 | 15 | 5 |
| A-176 | B-3 | C-4 | 80 | 15 | 5 |
| A-177 | B-3 | C-4 | 80 | 15 | 5 |
| A-178 | B-3 | C-4 | 80 | 15 | 5 |
| A-179 | B-3 | C-4 | 80 | 15 | 5 |
| A-180 | B-3 | C-4 | 80 | 15 | 5 |
| A-181 | B-3 | C-4 | 80 | 15 | 5 |
| A-182 | B-3 | C-4 | 80 | 15 | 5 |
| A-183 | B-3 | C-4 | 80 | 15 | 5 |
| A-190 | B-3 | C-4 | 80 | 15 | 5 |
| A-191 | B-3 | C-4 | 80 | 15 | 5 |
| A-192 | B-3 | C-4 | 80 | 15 | 5 |
| A-193 | B-3 | C-4 | 80 | 15 | 5 |
| A-194 | B-3 | C-4 | 80 | 15 | 5 |
| A-195 | B-3 | C-4 | 80 | 15 | 5 |
| A-196 | B-3 | C-4 | 80 | 15 | 5 |
| A-197 | B-3 | C-4 | 80 | 15 | 5 |
| A-198 | B-3 | C-4 | 80 | 15 | 5 |
| A-199 | B-3 | C-4 | 80 | 15 | 5 |

TABLE 48

| | | | Ratio of | | |
|---|---|---|---|---|---|
| | | | units | | |
| | | | (mol %) | | |
| A | B | C | A | B | C |
| A-200 | B-3 | C-4 | 80 | 15 | 5 |
| A-201 | B-3 | C-4 | 80 | 15 | 5 |
| A-202 | B-3 | C-4 | 80 | 15 | 5 |
| A-203 | B-3 | C-4 | 80 | 15 | 5 |
| A-204 | B-3 | C-4 | 80 | 15 | 5 |
| A-205 | B-3 | C-4 | 80 | 15 | 5 |
| A-206 | B-3 | C-4 | 80 | 15 | 5 |
| A-207 | B-3 | C-4 | 80 | 15 | 5 |
| A-208 | B-3 | C-4 | 80 | 15 | 5 |
| A-209 | B-3 | C-4 | 80 | 15 | 5 |
| A-210 | B-3 | C-4 | 80 | 15 | 5 |
| A-211 | B-3 | C-4 | 80 | 15 | 5 |
| A-212 | B-3 | C-4 | 80 | 15 | 5 |
| A-213 | B-3 | C-4 | 80 | 15 | 5 |
| A-220 | B-3 | C-4 | 80 | 15 | 5 |
| A-221 | B-3 | C-4 | 80 | 15 | 5 |
| A-222 | B-3 | C-4 | 80 | 15 | 5 |
| A-223 | B-3 | C-4 | 80 | 15 | 5 |
| A-224 | B-3 | C-4 | 80 | 15 | 5 |
| A-225 | B-3 | C-4 | 80 | 15 | 5 |
| A-226 | B-3 | C-4 | 80 | 15 | 5 |
| A-227 | B-3 | C-4 | 80 | 15 | 5 |
| A-228 | B-3 | C-4 | 80 | 15 | 5 |
| A-229 | B-3 | C-4 | 80 | 15 | 5 |
| A-230 | B-3 | C-4 | 80 | 15 | 5 |
| A-231 | B-3 | C-4 | 80 | 15 | 5 |
| A-232 | B-3 | C-4 | 80 | 15 | 5 |
| A-233 | B-3 | C-4 | 80 | 15 | 5 |
| A-234 | B-3 | C-4 | 80 | 15 | 5 |
| A-235 | B-3 | C-4 | 80 | 15 | 5 |
| A-236 | B-3 | C-4 | 80 | 15 | 5 |
| A-237 | B-3 | C-4 | 80 | 15 | 5 |
| A-238 | B-3 | C-4 | 80 | 15 | 5 |
| A-239 | B-3 | C-4 | 80 | 15 | 5 |
| A-240 | B-3 | C-4 | 80 | 15 | 5 |

TABLE 49

| | | | Ratio of | | |
|---|---|---|---|---|---|
| | | | units | | |
| | | | (mol %) | | |
| A | B | C | A | B | C |
| A-1 | B-1 | C-1 | 50 | 47 | 3 |
| A-2 | B-1 | C-1 | 50 | 47 | 3 |
| A-3 | B-1 | C-1 | 50 | 47 | 3 |
| A-4 | B-1 | C-1 | 50 | 47 | 3 |
| A-5 | B-1 | C-1 | 50 | 47 | 3 |
| A-6 | B-1 | C-1 | 50 | 47 | 3 |
| A-7 | B-1 | C-1 | 50 | 47 | 3 |
| A-8 | B-1 | C-1 | 50 | 47 | 3 |
| A-9 | B-1 | C-1 | 50 | 47 | 3 |
| A-10 | B-1 | C-1 | 50 | 47 | 3 |
| A-11 | B-1 | C-1 | 50 | 47 | 3 |
| A-12 | B-1 | C-1 | 50 | 47 | 3 |
| A-13 | B-1 | C-1 | 50 | 47 | 3 |
| A-14 | B-1 | C-1 | 50 | 47 | 3 |
| A-15 | B-1 | C-1 | 50 | 47 | 3 |
| A-16 | B-1 | C-1 | 50 | 47 | 3 |
| A-17 | B-1 | C-1 | 50 | 47 | 3 |
| A-18 | B-1 | C-1 | 50 | 47 | 3 |
| A-19 | B-1 | C-1 | 50 | 47 | 3 |
| A-20 | B-1 | C-1 | 50 | 47 | 3 |
| A-21 | B-1 | C-1 | 50 | 47 | 3 |
| A-22 | B-1 | C-1 | 50 | 47 | 3 |
| A-23 | B-1 | C-1 | 50 | 47 | 3 |
| A-24 | B-1 | C-1 | 50 | 47 | 3 |
| A-25 | B-1 | C-1 | 50 | 47 | 3 |
| A-26 | B-1 | C-1 | 50 | 47 | 3 |
| A-28 | B-1 | C-1 | 50 | 47 | 3 |
| A-29 | B-1 | C-1 | 50 | 47 | 3 |
| A-30 | B-1 | C-1 | 50 | 47 | 3 |
| A-31 | B-1 | C-1 | 50 | 47 | 3 |
| A-32 | B-1 | C-1 | 50 | 47 | 3 |
| A-33 | B-1 | C-1 | 50 | 47 | 3 |
| A-34 | B-1 | C-1 | 50 | 47 | 3 |
| A-35 | B-1 | C-1 | 50 | 47 | 3 |
| A-36 | B-1 | C-1 | 50 | 47 | 3 |
| A-37 | B-1 | C-1 | 50 | 47 | 3 |
| A-38 | B-1 | C-1 | 50 | 47 | 3 |
| A-39 | B-1 | C-1 | 50 | 47 | 3 |
| A-40 | B-1 | C-1 | 50 | 47 | 3 |
| A-41 | B-1 | C-1 | 50 | 47 | 3 |
| A-42 | B-1 | C-1 | 50 | 47 | 3 |
| A-43 | B-1 | C-1 | 50 | 47 | 3 |
| A-44 | B-1 | C-1 | 50 | 47 | 3 |
| A-45 | B-1 | C-1 | 50 | 47 | 3 |
| A-46 | B-1 | C-1 | 50 | 47 | 3 |
| A-47 | B-1 | C-1 | 50 | 47 | 3 |
| A-49 | B-1 | C-1 | 50 | 47 | 3 |
| A-50 | B-1 | C-1 | 50 | 47 | 3 |
| A-51 | B-1 | C-1 | 50 | 47 | 3 |
| A-52 | B-1 | C-1 | 50 | 47 | 3 |
| A-53 | B-1 | C-1 | 50 | 47 | 3 |
| A-54 | B-1 | C-1 | 50 | 47 | 3 |
| A-55 | B-1 | C-1 | 50 | 47 | 3 |
| A-56 | B-1 | C-1 | 50 | 47 | 3 |
| A-57 | B-1 | C-1 | 50 | 47 | 3 |
| A-58 | B-1 | C-1 | 50 | 47 | 3 |
| A-59 | B-1 | C-1 | 50 | 47 | 3 |
| A-60 | B-1 | C-1 | 50 | 47 | 3 |

TABLE 50

| | | | Ratio of | | |
|---|---|---|---|---|---|
| | | | units | | |
| | | | (mol %) | | |
| A | B | C | A | B | C |
| A-61 | B-1 | C-1 | 50 | 47 | 3 |
| A-62 | B-1 | C-1 | 50 | 47 | 3 |
| A-63 | B-1 | C-1 | 50 | 47 | 3 |
| A-64 | B-1 | C-1 | 50 | 47 | 3 |
| A-65 | B-1 | C-1 | 50 | 47 | 3 |
| A-66 | B-1 | C-1 | 50 | 47 | 3 |
| A-67 | B-1 | C-1 | 50 | 47 | 3 |
| A-68 | B-1 | C-1 | 50 | 47 | 3 |
| A-69 | B-1 | C-1 | 50 | 47 | 3 |
| A-70 | B-1 | C-1 | 50 | 47 | 3 |
| A-71 | B-1 | C-1 | 50 | 47 | 3 |
| A-72 | B-1 | C-1 | 50 | 47 | 3 |
| A-73 | B-1 | C-1 | 50 | 47 | 3 |
| A-74 | B-1 | C-1 | 50 | 47 | 3 |
| A-75 | B-1 | C-1 | 50 | 47 | 3 |
| A-76 | B-1 | C-1 | 50 | 47 | 3 |
| A-77 | B-1 | C-1 | 50 | 47 | 3 |
| A-78 | B-1 | C-1 | 50 | 47 | 3 |
| A-79 | B-1 | C-1 | 50 | 47 | 3 |
| A-80 | B-1 | C-1 | 50 | 47 | 3 |
| A-81 | B-1 | C-1 | 50 | 47 | 3 |
| A-82 | B-1 | C-1 | 50 | 47 | 3 |
| A-83 | B-1 | C-1 | 50 | 47 | 3 |
| A-84 | B-1 | C-1 | 50 | 47 | 3 |
| A-85 | B-1 | C-1 | 50 | 47 | 3 |
| A-86 | B-1 | C-1 | 50 | 47 | 3 |
| A-87 | B-1 | C-1 | 50 | 47 | 3 |
| A-88 | B-1 | C-1 | 50 | 47 | 3 |
| A-89 | B-1 | C-1 | 50 | 47 | 3 |
| A-90 | B-1 | C-1 | 50 | 47 | 3 |
| A-91 | B-1 | C-1 | 50 | 47 | 3 |
| A-92 | B-1 | C-1 | 50 | 47 | 3 |
| A-93 | B-1 | C-1 | 50 | 47 | 3 |
| A-94 | B-1 | C-1 | 50 | 47 | 3 |
| A-95 | B-1 | C-1 | 50 | 47 | 3 |
| A-96 | B-1 | C-1 | 50 | 47 | 3 |
| A-100 | B-1 | C-1 | 50 | 47 | 3 |
| A-101 | B-1 | C-1 | 50 | 47 | 3 |
| A-102 | B-1 | C-1 | 50 | 47 | 3 |
| A-103 | B-1 | C-1 | 50 | 47 | 3 |
| A-104 | B-1 | C-1 | 50 | 47 | 3 |
| A-105 | B-1 | C-1 | 50 | 47 | 3 |
| A-106 | B-1 | C-1 | 50 | 47 | 3 |
| A-107 | B-1 | C-1 | 50 | 47 | 3 |
| A-108 | B-1 | C-1 | 50 | 47 | 3 |
| A-109 | B-1 | C-1 | 50 | 47 | 3 |
| A-110 | B-1 | C-1 | 50 | 47 | 3 |
| A-111 | B-1 | C-1 | 50 | 47 | 3 |
| A-112 | B-1 | C-1 | 50 | 47 | 3 |
| A-113 | B-1 | C-1 | 50 | 47 | 3 |
| A-114 | B-1 | C-1 | 50 | 47 | 3 |
| A-115 | B-1 | C-1 | 50 | 47 | 3 |
| A-116 | B-1 | C-1 | 50 | 47 | 3 |
| A-117 | B-1 | C-1 | 50 | 47 | 3 |
| A-118 | B-1 | C-1 | 50 | 47 | 3 |
| A-119 | B-1 | C-1 | 50 | 47 | 3 |
| A-120 | B-1 | C-1 | 50 | 47 | 3 |
| A-121 | B-1 | C-1 | 50 | 47 | 3 |
| A-122 | B-1 | C-1 | 50 | 47 | 3 |
| A-123 | B-1 | C-1 | 50 | 47 | 3 |

TABLE 51

| | | | Ratio of | | |
|---|---|---|---|---|---|
| | | | units | | |
| | | | (mol %) | | |
| A | B | C | A | B | C |
| A-124 | B-1 | C-1 | 50 | 47 | 3 |
| A-125 | B-1 | C-1 | 50 | 47 | 3 |
| A-126 | B-1 | C-1 | 50 | 47 | 3 |
| A-127 | B-1 | C-1 | 50 | 47 | 3 |
| A-128 | B-1 | C-1 | 50 | 47 | 3 |
| A-129 | B-1 | C-1 | 50 | 47 | 3 |
| A-130 | B-1 | C-1 | 50 | 47 | 3 |
| A-131 | B-1 | C-1 | 50 | 47 | 3 |
| A-132 | B-1 | C-1 | 50 | 47 | 3 |
| A-133 | B-1 | C-1 | 50 | 47 | 3 |
| A-134 | B-1 | C-1 | 50 | 47 | 3 |
| A-135 | B-1 | C-1 | 50 | 47 | 3 |
| A-136 | B-1 | C-1 | 50 | 47 | 3 |
| A-137 | B-1 | C-1 | 50 | 47 | 3 |
| A-138 | B-1 | C-1 | 50 | 47 | 3 |
| A-139 | B-1 | C-1 | 50 | 47 | 3 |
| A-140 | B-1 | C-1 | 50 | 47 | 3 |
| A-141 | B-1 | C-1 | 50 | 47 | 3 |
| A-142 | B-1 | C-1 | 50 | 47 | 3 |
| A-143 | B-1 | C-1 | 50 | 47 | 3 |
| A-144 | B-1 | C-1 | 50 | 47 | 3 |
| A-145 | B-1 | C-1 | 50 | 47 | 3 |
| A-146 | B-1 | C-1 | 50 | 47 | 3 |
| A-147 | B-1 | C-1 | 50 | 47 | 3 |
| A-160 | B-1 | C-1 | 50 | 47 | 3 |
| A-161 | B-1 | C-1 | 50 | 47 | 3 |
| A-162 | B-1 | C-1 | 50 | 47 | 3 |
| A-163 | B-1 | C-1 | 50 | 47 | 3 |
| A-164 | B-1 | C-1 | 50 | 47 | 3 |
| A-165 | B-1 | C-1 | 50 | 47 | 3 |
| A-166 | B-1 | C-1 | 50 | 47 | 3 |
| A-167 | B-1 | C-1 | 50 | 47 | 3 |
| A-168 | B-1 | C-1 | 50 | 47 | 3 |
| A-169 | B-1 | C-1 | 50 | 47 | 3 |
| A-170 | B-1 | C-1 | 50 | 47 | 3 |
| A-171 | B-1 | C-1 | 50 | 47 | 3 |
| A-172 | B-1 | C-1 | 50 | 47 | 3 |
| A-173 | B-1 | C-1 | 50 | 47 | 3 |
| A-174 | B-1 | C-1 | 50 | 47 | 3 |
| A-175 | B-1 | C-1 | 50 | 47 | 3 |
| A-176 | B-1 | C-1 | 50 | 47 | 3 |
| A-177 | B-1 | C-1 | 50 | 47 | 3 |
| A-178 | B-1 | C-1 | 50 | 47 | 3 |
| A-179 | B-1 | C-1 | 50 | 47 | 3 |
| A-180 | B-1 | C-1 | 50 | 47 | 3 |
| A-181 | B-1 | C-1 | 50 | 47 | 3 |
| A-182 | B-1 | C-1 | 50 | 47 | 3 |
| A-183 | B-1 | C-1 | 50 | 47 | 3 |
| A-190 | B-1 | C-1 | 50 | 47 | 3 |
| A-191 | B-1 | C-1 | 50 | 47 | 3 |
| A-192 | B-1 | C-1 | 50 | 47 | 3 |
| A-193 | B-1 | C-1 | 50 | 47 | 3 |
| A-194 | B-1 | C-1 | 50 | 47 | 3 |
| A-195 | B-1 | C-1 | 50 | 47 | 3 |
| A-196 | B-1 | C-1 | 50 | 47 | 3 |
| A-197 | B-1 | C-1 | 50 | 47 | 3 |
| A-198 | B-1 | C-1 | 50 | 47 | 3 |
| A-199 | B-1 | C-1 | 50 | 47 | 3 |
| A-200 | B-1 | C-1 | 50 | 47 | 3 |
| A-201 | B-1 | C-1 | 50 | 47 | 3 |
| A-202 | B-1 | C-1 | 50 | 47 | 3 |
| A-203 | B-1 | C-1 | 50 | 47 | 3 |
| A-204 | B-1 | C-1 | 50 | 47 | 3 |
| A-205 | B-1 | C-1 | 50 | 47 | 3 |
| A-206 | B-1 | C-1 | 50 | 47 | 3 |
| A-207 | B-1 | C-1 | 50 | 47 | 3 |
| A-208 | B-1 | C-1 | 50 | 47 | 3 |
| A-209 | B-1 | C-1 | 50 | 47 | 3 |
| A-210 | B-1 | C-1 | 50 | 47 | 3 |
| A-211 | B-1 | C-1 | 50 | 47 | 3 |
| A-212 | B-1 | C-1 | 50 | 47 | 3 |
| A-213 | B-1 | C-1 | 50 | 47 | 3 |

TABLE 52

| | | | Ratio of | | |
|---|---|---|---|---|---|
| | | | units | | |
| | | | (mol %) | | |
| A | B | C | A | B | C |
| A-220 | B-1 | C-1 | 50 | 47 | 3 |
| A-221 | B-1 | C-1 | 50 | 47 | 3 |
| A-222 | B-1 | C-1 | 50 | 47 | 3 |
| A-223 | B-1 | C-1 | 50 | 47 | 3 |
| A-224 | B-1 | C-1 | 50 | 47 | 3 |
| A-225 | B-1 | C-1 | 50 | 47 | 3 |
| A-226 | B-1 | C-1 | 50 | 47 | 3 |
| A-227 | B-1 | C-1 | 50 | 47 | 3 |
| A-228 | B-1 | C-1 | 50 | 47 | 3 |
| A-229 | B-1 | C-1 | 50 | 47 | 3 |
| A-230 | B-1 | C-1 | 50 | 47 | 3 |
| A-231 | B-1 | C-1 | 50 | 47 | 3 |
| A-232 | B-1 | C-1 | 50 | 47 | 3 |
| A-233 | B-1 | C-1 | 50 | 47 | 3 |
| A-234 | B-1 | C-1 | 50 | 47 | 3 |
| A-235 | B-1 | C-1 | 50 | 47 | 3 |
| A-236 | B-1 | C-1 | 50 | 47 | 3 |
| A-237 | B-1 | C-1 | 50 | 47 | 3 |
| A-238 | B-1 | C-1 | 50 | 47 | 3 |
| A-239 | B-1 | C-1 | 50 | 47 | 3 |
| A-240 | B-1 | C-1 | 50 | 47 | 3 |

TABLE 53

| | | | Ratio of | | |
|---|---|---|---|---|---|
| | | | units | | |
| | | | (mol %) | | |
| A | B | C | A | B | C |
| A-1 | B-1 | C-2 | 50 | 47 | 3 |
| A-2 | B-1 | C-2 | 50 | 47 | 3 |
| A-3 | B-1 | C-2 | 50 | 47 | 3 |
| A-4 | B-1 | C-2 | 50 | 47 | 3 |
| A-5 | B-1 | C-2 | 50 | 47 | 3 |
| A-6 | B-1 | C-2 | 50 | 47 | 3 |
| A-7 | B-1 | C-2 | 50 | 47 | 3 |
| A-8 | B-1 | C-2 | 50 | 47 | 3 |
| A-9 | B-1 | C-2 | 50 | 47 | 3 |
| A-10 | B-1 | C-2 | 50 | 47 | 3 |
| A-11 | B-1 | C-2 | 50 | 47 | 3 |
| A-12 | B-1 | C-2 | 50 | 47 | 3 |
| A-13 | B-1 | C-2 | 50 | 47 | 3 |
| A-14 | B-1 | C-2 | 50 | 47 | 3 |
| A-15 | B-1 | C-2 | 50 | 47 | 3 |
| A-16 | B-1 | C-2 | 50 | 47 | 3 |
| A-17 | B-1 | C-2 | 50 | 47 | 3 |
| A-18 | B-1 | C-2 | 50 | 47 | 3 |
| A-19 | B-1 | C-2 | 50 | 47 | 3 |
| A-20 | B-1 | C-2 | 50 | 47 | 3 |
| A-21 | B-1 | C-2 | 50 | 47 | 3 |
| A-22 | B-1 | C-2 | 50 | 47 | 3 |
| A-23 | B-1 | C-2 | 50 | 47 | 3 |
| A-24 | B-1 | C-2 | 50 | 47 | 3 |
| A-25 | B-1 | C-2 | 50 | 47 | 3 |
| A-26 | B-1 | C-2 | 50 | 47 | 3 |
| A-28 | B-1 | C-2 | 50 | 47 | 3 |
| A-29 | B-1 | C-2 | 50 | 47 | 3 |
| A-30 | B-1 | C-2 | 50 | 47 | 3 |
| A-31 | B-1 | C-2 | 50 | 47 | 3 |
| A-32 | B-1 | C-2 | 50 | 47 | 3 |
| A-33 | B-1 | C-2 | 50 | 47 | 3 |
| A-34 | B-1 | C-2 | 50 | 47 | 3 |
| A-35 | B-1 | C-2 | 50 | 47 | 3 |
| A-36 | B-1 | C-2 | 50 | 47 | 3 |
| A-37 | B-1 | C-2 | 50 | 47 | 3 |
| A-38 | B-1 | C-2 | 50 | 47 | 3 |
| A-39 | B-1 | C-2 | 50 | 47 | 3 |
| A-40 | B-1 | C-2 | 50 | 47 | 3 |
| A-41 | B-1 | C-2 | 50 | 47 | 3 |
| A-42 | B-1 | C-2 | 50 | 47 | 3 |
| A-43 | B-1 | C-2 | 50 | 47 | 3 |
| A-44 | B-1 | C-2 | 50 | 47 | 3 |
| A-45 | B-1 | C-2 | 50 | 47 | 3 |
| A-46 | B-1 | C-2 | 50 | 47 | 3 |
| A-47 | B-1 | C-2 | 50 | 47 | 3 |
| A-49 | B-1 | C-2 | 50 | 47 | 3 |
| A-50 | B-1 | C-2 | 50 | 47 | 3 |
| A-51 | B-1 | C-2 | 50 | 47 | 3 |
| A-52 | B-1 | C-2 | 50 | 47 | 3 |
| A-53 | B-1 | C-2 | 50 | 47 | 3 |
| A-54 | B-1 | C-2 | 50 | 47 | 3 |
| A-55 | B-1 | C-2 | 50 | 47 | 3 |
| A-56 | B-1 | C-2 | 50 | 47 | 3 |
| A-57 | B-1 | C-2 | 50 | 47 | 3 |
| A-58 | B-1 | C-2 | 50 | 47 | 3 |
| A-59 | B-1 | C-2 | 50 | 47 | 3 |
| A-60 | B-1 | C-2 | 50 | 47 | 3 |

TABLE 54

| | | | Ratio | | |
|---|---|---|---|---|---|
| | | | of units | | |
| | | | (mol %) | | |
| A | B | C | A | B | C |
| A-61 | B-1 | C-2 | 50 | 47 | 3 |
| A-62 | B-1 | C-2 | 50 | 47 | 3 |
| A-63 | B-1 | C-2 | 50 | 47 | 3 |
| A-64 | B-1 | C-2 | 50 | 47 | 3 |
| A-65 | B-1 | C-2 | 50 | 47 | 3 |
| A-66 | B-1 | C-2 | 50 | 47 | 3 |
| A-67 | B-1 | C-2 | 50 | 47 | 3 |
| A-68 | B-1 | C-2 | 50 | 47 | 3 |
| A-69 | B-1 | C-2 | 50 | 47 | 3 |
| A-70 | B-1 | C-2 | 50 | 47 | 3 |
| A-71 | B-1 | C-2 | 50 | 47 | 3 |
| A-72 | B-1 | C-2 | 50 | 47 | 3 |
| A-73 | B-1 | C-2 | 50 | 47 | 3 |
| A-74 | B-1 | C-2 | 50 | 47 | 3 |
| A-75 | B-1 | C-2 | 50 | 47 | 3 |
| A-76 | B-1 | C-2 | 50 | 47 | 3 |
| A-77 | B-1 | C-2 | 50 | 47 | 3 |
| A-78 | B-1 | C-2 | 50 | 47 | 3 |
| A-79 | B-1 | C-2 | 50 | 47 | 3 |
| A-80 | B-1 | C-2 | 50 | 47 | 3 |
| A-81 | B-1 | C-2 | 50 | 47 | 3 |
| A-82 | B-1 | C-2 | 50 | 47 | 3 |
| A-83 | B-1 | C-2 | 50 | 47 | 3 |
| A-84 | B-1 | C-2 | 50 | 47 | 3 |
| A-85 | B-1 | C-2 | 50 | 47 | 3 |
| A-86 | B-1 | C-2 | 50 | 47 | 3 |
| A-87 | B-1 | C-2 | 50 | 47 | 3 |
| A-88 | B-1 | C-2 | 50 | 47 | 3 |
| A-89 | B-1 | C-2 | 50 | 47 | 3 |
| A-90 | B-1 | C-2 | 50 | 47 | 3 |
| A-91 | B-1 | C-2 | 50 | 47 | 3 |
| A-92 | B-1 | C-2 | 50 | 47 | 3 |
| A-93 | B-1 | C-2 | 50 | 47 | 3 |
| A-94 | B-1 | C-2 | 50 | 47 | 3 |
| A-95 | B-1 | C-2 | 50 | 47 | 3 |
| A-96 | B-1 | C-2 | 50 | 47 | 3 |
| A-100 | B-1 | C-2 | 50 | 47 | 3 |
| A-101 | B-1 | C-2 | 50 | 47 | 3 |
| A-102 | B-1 | C-2 | 50 | 47 | 3 |
| A-103 | B-1 | C-2 | 50 | 47 | 3 |
| A-104 | B-1 | C-2 | 50 | 47 | 3 |
| A-105 | B-1 | C-2 | 50 | 47 | 3 |
| A-106 | B-1 | C-2 | 50 | 47 | 3 |
| A-107 | B-1 | C-2 | 50 | 47 | 3 |
| A-108 | B-1 | C-2 | 50 | 47 | 3 |
| A-109 | B-1 | C-2 | 50 | 47 | 3 |
| A-110 | B-1 | C-2 | 50 | 47 | 3 |
| A-111 | B-1 | C-2 | 50 | 47 | 3 |
| A-112 | B-1 | C-2 | 50 | 47 | 3 |
| A-113 | B-1 | C-2 | 50 | 47 | 3 |
| A-114 | B-1 | C-2 | 50 | 47 | 3 |
| A-115 | B-1 | C-2 | 50 | 47 | 3 |
| A-116 | B-1 | C-2 | 50 | 47 | 3 |
| A-117 | B-1 | C-2 | 50 | 47 | 3 |
| A-118 | B-1 | C-2 | 50 | 47 | 3 |
| A-119 | B-1 | C-2 | 50 | 47 | 3 |
| A-120 | B-1 | C-2 | 50 | 47 | 3 |
| A-121 | B-1 | C-2 | 50 | 47 | 3 |
| A-122 | B-1 | C-2 | 50 | 47 | 3 |
| A-123 | B-1 | C-2 | 50 | 47 | 3 |

TABLE 55

| | | | Ratio of | | |
|---|---|---|---|---|---|
| | | | units | | |
| | | | (mol %) | | |
| A | B | C | A | B | C |
| A-124 | B-1 | C-2 | 50 | 47 | 3 |
| A-125 | B-1 | C-2 | 50 | 47 | 3 |
| A-126 | B-1 | C-2 | 50 | 47 | 3 |
| A-127 | B-1 | C-2 | 50 | 47 | 3 |
| A-128 | B-1 | C-2 | 50 | 47 | 3 |
| A-129 | B-1 | C-2 | 50 | 47 | 3 |
| A-130 | B-1 | C-2 | 50 | 47 | 3 |
| A-131 | B-1 | C-2 | 50 | 47 | 3 |
| A-132 | B-1 | C-2 | 50 | 47 | 3 |
| A-133 | B-1 | C-2 | 50 | 47 | 3 |
| A-134 | B-1 | C-2 | 50 | 47 | 3 |
| A-135 | B-1 | C-2 | 50 | 47 | 3 |
| A-136 | B-1 | C-2 | 50 | 47 | 3 |
| A-137 | B-1 | C-2 | 50 | 47 | 3 |
| A-138 | B-1 | C-2 | 50 | 47 | 3 |
| A-139 | B-1 | C-2 | 50 | 47 | 3 |
| A-140 | B-1 | C-2 | 50 | 47 | 3 |
| A-141 | B-1 | C-2 | 50 | 47 | 3 |
| A-142 | B-1 | C-2 | 50 | 47 | 3 |
| A-143 | B-1 | C-2 | 50 | 47 | 3 |
| A-144 | B-1 | C-2 | 50 | 47 | 3 |
| A-145 | B-1 | C-2 | 50 | 47 | 3 |
| A-146 | B-1 | C-2 | 50 | 47 | 3 |
| A-147 | B-1 | C-2 | 50 | 47 | 3 |
| A-160 | B-1 | C-2 | 50 | 47 | 3 |
| A-161 | B-1 | C-2 | 50 | 47 | 3 |
| A-162 | B-1 | C-2 | 50 | 47 | 3 |
| A-163 | B-1 | C-2 | 50 | 47 | 3 |
| A-164 | B-1 | C-2 | 50 | 47 | 3 |
| A-165 | B-1 | C-2 | 50 | 47 | 3 |
| A-166 | B-1 | C-2 | 50 | 47 | 3 |
| A-167 | B-1 | C-2 | 50 | 47 | 3 |
| A-168 | B-1 | C-2 | 50 | 47 | 3 |
| A-169 | B-1 | C-2 | 50 | 47 | 3 |
| A-170 | B-1 | C-2 | 50 | 47 | 3 |
| A-171 | B-1 | C-2 | 50 | 47 | 3 |
| A-172 | B-1 | C-2 | 50 | 47 | 3 |
| A-173 | B-1 | C-2 | 50 | 47 | 3 |
| A-174 | B-1 | C-2 | 50 | 47 | 3 |
| A-175 | B-1 | C-2 | 50 | 47 | 3 |
| A-176 | B-1 | C-2 | 50 | 47 | 3 |
| A-177 | B-1 | C-2 | 50 | 47 | 3 |
| A-178 | B-1 | C-2 | 50 | 47 | 3 |
| A-179 | B-1 | C-2 | 50 | 47 | 3 |
| A-180 | B-1 | C-2 | 50 | 47 | 3 |
| A-181 | B-1 | C-2 | 50 | 47 | 3 |
| A-182 | B-1 | C-2 | 50 | 47 | 3 |
| A-183 | B-1 | C-2 | 50 | 47 | 3 |
| A-190 | B-1 | C-2 | 50 | 47 | 3 |
| A-191 | B-1 | C-2 | 50 | 47 | 3 |
| A-192 | B-1 | C-2 | 50 | 47 | 3 |
| A-193 | B-1 | C-2 | 50 | 47 | 3 |
| A-194 | B-1 | C-2 | 50 | 47 | 3 |
| A-195 | B-1 | C-2 | 50 | 47 | 3 |
| A-196 | B-1 | C-2 | 50 | 47 | 3 |
| A-197 | B-1 | C-2 | 50 | 47 | 3 |
| A-198 | B-1 | C-2 | 50 | 47 | 3 |
| A-199 | B-1 | C-2 | 50 | 47 | 3 |
| A-200 | B-1 | C-2 | 50 | 47 | 3 |
| A-201 | B-1 | C-2 | 50 | 47 | 3 |
| A-202 | B-1 | C-2 | 50 | 47 | 3 |
| A-203 | B-1 | C-2 | 50 | 47 | 3 |
| A-204 | B-1 | C-2 | 50 | 47 | 3 |
| A-205 | B-1 | C-2 | 50 | 47 | 3 |
| A-206 | B-1 | C-2 | 50 | 47 | 3 |
| A-207 | B-1 | C-2 | 50 | 47 | 3 |
| A-208 | B-1 | C-2 | 50 | 47 | 3 |
| A-209 | B-1 | C-2 | 50 | 47 | 3 |
| A-210 | B-1 | C-2 | 50 | 47 | 3 |
| A-211 | B-1 | C-2 | 50 | 47 | 3 |
| A-212 | B-1 | C-2 | 50 | 47 | 3 |
| A-213 | B-1 | C-2 | 50 | 47 | 3 |

TABLE 56

| | | | Ratio of | | |
|---|---|---|---|---|---|
| | | | units | | |
| | | | (mol %) | | |
| A | B | C | A | B | C |
| A-220 | B-1 | C-2 | 50 | 47 | 3 |
| A-221 | B-1 | C-2 | 50 | 47 | 3 |
| A-222 | B-1 | C-2 | 50 | 47 | 3 |
| A-223 | B-1 | C-2 | 50 | 47 | 3 |
| A-224 | B-1 | C-2 | 50 | 47 | 3 |
| A-225 | B-1 | C-2 | 50 | 47 | 3 |
| A-226 | B-1 | C-2 | 50 | 47 | 3 |
| A-227 | B-1 | C-2 | 50 | 47 | 3 |
| A-228 | B-1 | C-2 | 50 | 47 | 3 |
| A-229 | B-1 | C-2 | 50 | 47 | 3 |
| A-230 | B-1 | C-2 | 50 | 47 | 3 |
| A-231 | B-1 | C-2 | 50 | 47 | 3 |
| A-232 | B-1 | C-2 | 50 | 47 | 3 |
| A-233 | B-1 | C-2 | 50 | 47 | 3 |
| A-234 | B-1 | C-2 | 50 | 47 | 3 |
| A-235 | B-1 | C-2 | 50 | 47 | 3 |
| A-236 | B-1 | C-2 | 50 | 47 | 3 |
| A-237 | B-1 | C-2 | 50 | 47 | 3 |
| A-238 | B-1 | C-2 | 50 | 47 | 3 |
| A-239 | B-1 | C-2 | 50 | 47 | 3 |
| A-240 | B-1 | C-2 | 50 | 47 | 3 |

TABLE 57

| | | | Ratio of | | |
|---|---|---|---|---|---|
| | | | units | | |
| | | | (mol %) | | |
| A | B | C | A | B | C |
| A-1 | B-1 | C-3 | 50 | 30 | 20 |
| A-2 | B-1 | C-3 | 50 | 30 | 20 |
| A-3 | B-1 | C-3 | 50 | 30 | 20 |
| A-4 | B-1 | C-3 | 50 | 30 | 20 |
| A-5 | B-1 | C-3 | 50 | 30 | 20 |
| A-6 | B-1 | C-3 | 50 | 30 | 20 |
| A-7 | B-1 | C-3 | 50 | 30 | 20 |
| A-8 | B-1 | C-3 | 50 | 30 | 20 |
| A-9 | B-1 | C-3 | 50 | 30 | 20 |
| A-10 | B-1 | C-3 | 50 | 30 | 20 |
| A-11 | B-1 | C-3 | 50 | 30 | 20 |
| A-12 | B-1 | C-3 | 50 | 30 | 20 |
| A-13 | B-1 | C-3 | 50 | 30 | 20 |
| A-14 | B-1 | C-3 | 50 | 30 | 20 |
| A-15 | B-1 | C-3 | 50 | 30 | 20 |
| A-16 | B-1 | C-3 | 50 | 30 | 20 |
| A-17 | B-1 | C-3 | 50 | 30 | 20 |
| A-18 | B-1 | C-3 | 50 | 30 | 20 |
| A-19 | B-1 | C-3 | 50 | 30 | 20 |
| A-20 | B-1 | C-3 | 50 | 30 | 20 |
| A-21 | B-1 | C-3 | 50 | 30 | 20 |
| A-22 | B-1 | C-3 | 50 | 30 | 20 |
| A-23 | B-1 | C-3 | 50 | 30 | 20 |
| A-24 | B-1 | C-3 | 50 | 30 | 20 |
| A-25 | B-1 | C-3 | 50 | 30 | 20 |
| A-26 | B-1 | C-3 | 50 | 30 | 20 |
| A-28 | B-1 | C-3 | 50 | 30 | 20 |
| A-29 | B-1 | C-3 | 50 | 30 | 20 |
| A-30 | B-1 | C-3 | 50 | 30 | 20 |
| A-31 | B-1 | C-3 | 50 | 30 | 20 |
| A-32 | B-1 | C-3 | 50 | 30 | 20 |
| A-33 | B-1 | C-3 | 50 | 30 | 20 |
| A-34 | B-1 | C-3 | 50 | 30 | 20 |
| A-35 | B-1 | C-3 | 50 | 30 | 20 |
| A-36 | B-1 | C-3 | 50 | 30 | 20 |
| A-37 | B-1 | C-3 | 50 | 30 | 20 |
| A-38 | B-1 | C-3 | 50 | 30 | 20 |
| A-39 | B-1 | C-3 | 50 | 30 | 20 |
| A-40 | B-1 | C-3 | 50 | 30 | 20 |
| A-41 | B-1 | C-3 | 50 | 30 | 20 |
| A-42 | B-1 | C-3 | 50 | 30 | 20 |
| A-43 | B-1 | C-3 | 50 | 30 | 20 |
| A-44 | B-1 | C-3 | 50 | 30 | 20 |
| A-45 | B-1 | C-3 | 50 | 30 | 20 |
| A-46 | B-1 | C-3 | 50 | 30 | 20 |
| A-47 | B-1 | C-3 | 50 | 30 | 20 |
| A-49 | B-1 | C-3 | 50 | 30 | 20 |
| A-50 | B-1 | C-3 | 50 | 30 | 20 |
| A-51 | B-1 | C-3 | 50 | 30 | 20 |
| A-52 | B-1 | C-3 | 50 | 30 | 20 |
| A-53 | B-1 | C-3 | 50 | 30 | 20 |
| A-54 | B-1 | C-3 | 50 | 30 | 20 |
| A-55 | B-1 | C-3 | 50 | 30 | 20 |
| A-56 | B-1 | C-3 | 50 | 30 | 20 |
| A-57 | B-1 | C-3 | 50 | 30 | 20 |
| A-58 | B-1 | C-3 | 50 | 30 | 20 |
| A-59 | B-1 | C-3 | 50 | 30 | 20 |
| A-60 | B-1 | C-3 | 50 | 30 | 20 |

TABLE 58

| | | | Ratio of | | |
|---|---|---|---|---|---|
| | | | units | | |
| | | | (mol %) | | |
| A | B | C | A | B | C |
| A-61 | B-1 | C-3 | 50 | 30 | 20 |
| A-62 | B-1 | C-3 | 50 | 30 | 20 |
| A-63 | B-1 | C-3 | 50 | 30 | 20 |
| A-64 | B-1 | C-3 | 50 | 30 | 20 |
| A-65 | B-1 | C-3 | 50 | 30 | 20 |
| A-66 | B-1 | C-3 | 50 | 30 | 20 |
| A-67 | B-1 | C-3 | 50 | 30 | 20 |
| A-68 | B-1 | C-3 | 50 | 30 | 20 |
| A-69 | B-1 | C-3 | 50 | 30 | 20 |
| A-70 | B-1 | C-3 | 50 | 30 | 20 |
| A-71 | B-1 | C-3 | 50 | 30 | 20 |
| A-72 | B-1 | C-3 | 50 | 30 | 20 |
| A-73 | B-1 | C-3 | 50 | 30 | 20 |
| A-74 | B-1 | C-3 | 50 | 30 | 20 |
| A-75 | B-1 | C-3 | 50 | 30 | 20 |
| A-76 | B-1 | C-3 | 50 | 30 | 20 |
| A-77 | B-1 | C-3 | 50 | 30 | 20 |
| A-78 | B-1 | C-3 | 50 | 30 | 20 |
| A-79 | B-1 | C-3 | 50 | 30 | 20 |
| A-80 | B-1 | C-3 | 50 | 30 | 20 |
| A-81 | B-1 | C-3 | 50 | 30 | 20 |
| A-82 | B-1 | C-3 | 50 | 30 | 20 |
| A-83 | B-1 | C-3 | 50 | 30 | 20 |
| A-84 | B-1 | C-3 | 50 | 30 | 20 |
| A-85 | B-1 | C-3 | 50 | 30 | 20 |
| A-86 | B-1 | C-3 | 50 | 30 | 20 |
| A-87 | B-1 | C-3 | 50 | 30 | 20 |
| A-88 | B-1 | C-3 | 50 | 30 | 20 |
| A-89 | B-1 | C-3 | 50 | 30 | 20 |
| A-90 | B-1 | C-3 | 50 | 30 | 20 |
| A-91 | B-1 | C-3 | 50 | 30 | 20 |
| A-92 | B-1 | C-3 | 50 | 30 | 20 |
| A-93 | B-1 | C-3 | 50 | 30 | 20 |
| A-94 | B-1 | C-3 | 50 | 30 | 20 |
| A-95 | B-1 | C-3 | 50 | 30 | 20 |
| A-96 | B-1 | C-3 | 50 | 30 | 20 |
| A-100 | B-1 | C-3 | 50 | 30 | 20 |
| A-101 | B-1 | C-3 | 50 | 30 | 20 |
| A-102 | B-1 | C-3 | 50 | 30 | 20 |
| A-103 | B-1 | C-3 | 50 | 30 | 20 |
| A-104 | B-1 | C-3 | 50 | 30 | 20 |
| A-105 | B-1 | C-3 | 50 | 30 | 20 |
| A-106 | B-1 | C-3 | 50 | 30 | 20 |
| A-107 | B-1 | C-3 | 50 | 30 | 20 |
| A-108 | B-1 | C-3 | 50 | 30 | 20 |
| A-109 | B-1 | C-3 | 50 | 30 | 20 |
| A-110 | B-1 | C-3 | 50 | 30 | 20 |
| A-111 | B-1 | C-3 | 50 | 30 | 20 |
| A-112 | B-1 | C-3 | 50 | 30 | 20 |
| A-113 | B-1 | C-3 | 50 | 30 | 20 |
| A-114 | B-1 | C-3 | 50 | 30 | 20 |
| A-115 | B-1 | C-3 | 50 | 30 | 20 |
| A-116 | B-1 | C-3 | 50 | 30 | 20 |
| A-117 | B-1 | C-3 | 50 | 30 | 20 |
| A-118 | B-1 | C-3 | 50 | 30 | 20 |
| A-119 | B-1 | C-3 | 50 | 30 | 20 |
| A-120 | B-1 | C-3 | 50 | 30 | 20 |
| A-121 | B-1 | C-3 | 50 | 30 | 20 |
| A-122 | B-1 | C-3 | 50 | 30 | 20 |
| A-123 | B-1 | C-3 | 50 | 30 | 20 |

TABLE 59

| | | | Ratio of | | |
|---|---|---|---|---|---|
| | | | units | | |
| | | | (mol %) | | |
| A | B | C | A | B | C |
| A-124 | B-1 | C-3 | 50 | 30 | 20 |
| A-125 | B-1 | C-3 | 50 | 30 | 20 |
| A-126 | B-1 | C-3 | 50 | 30 | 20 |
| A-127 | B-1 | C-3 | 50 | 30 | 20 |
| A-128 | B-1 | C-3 | 50 | 30 | 20 |
| A-129 | B-1 | C-3 | 50 | 30 | 20 |
| A-130 | B-1 | C-3 | 50 | 30 | 20 |
| A-131 | B-1 | C-3 | 50 | 30 | 20 |
| A-132 | B-1 | C-3 | 50 | 30 | 20 |
| A-133 | B-1 | C-3 | 50 | 30 | 20 |
| A-134 | B-1 | C-3 | 50 | 30 | 20 |
| A-135 | B-1 | C-3 | 50 | 30 | 20 |
| A-136 | B-1 | C-3 | 50 | 30 | 20 |
| A-137 | B-1 | C-3 | 50 | 30 | 20 |
| A-138 | B-1 | C-3 | 50 | 30 | 20 |
| A-139 | B-1 | C-3 | 50 | 30 | 20 |
| A-140 | B-1 | C-3 | 50 | 30 | 20 |
| A-141 | B-1 | C-3 | 50 | 30 | 20 |
| A-142 | B-1 | C-3 | 50 | 30 | 20 |
| A-143 | B-1 | C-3 | 50 | 30 | 20 |
| A-144 | B-1 | C-3 | 50 | 30 | 20 |
| A-145 | B-1 | C-3 | 50 | 30 | 20 |
| A-146 | B-1 | C-3 | 50 | 30 | 20 |
| A-147 | B-1 | C-3 | 50 | 30 | 20 |
| A-160 | B-1 | C-3 | 50 | 30 | 20 |
| A-161 | B-1 | C-3 | 50 | 30 | 20 |
| A-162 | B-1 | C-3 | 50 | 30 | 20 |
| A-163 | B-1 | C-3 | 50 | 30 | 20 |
| A-164 | B-1 | C-3 | 50 | 30 | 20 |
| A-165 | B-1 | C-3 | 50 | 30 | 20 |
| A-166 | B-1 | C-3 | 50 | 30 | 20 |
| A-167 | B-1 | C-3 | 50 | 30 | 20 |
| A-168 | B-1 | C-3 | 50 | 30 | 20 |
| A-169 | B-1 | C-3 | 50 | 30 | 20 |
| A-170 | B-1 | C-3 | 50 | 30 | 20 |
| A-171 | B-1 | C-3 | 50 | 30 | 20 |
| A-172 | B-1 | C-3 | 50 | 30 | 20 |
| A-173 | B-1 | C-3 | 50 | 30 | 20 |
| A-174 | B-1 | C-3 | 50 | 30 | 20 |
| A-175 | B-1 | C-3 | 50 | 30 | 20 |
| A-176 | B-1 | C-3 | 50 | 30 | 20 |
| A-177 | B-1 | C-3 | 50 | 30 | 20 |
| A-178 | B-1 | C-3 | 50 | 30 | 20 |
| A-179 | B-1 | C-3 | 50 | 30 | 20 |
| A-180 | B-1 | C-3 | 50 | 30 | 20 |
| A-181 | B-1 | C-3 | 50 | 30 | 20 |
| A-182 | B-1 | C-3 | 50 | 30 | 20 |
| A-183 | B-1 | C-3 | 50 | 30 | 20 |
| A-190 | B-1 | C-3 | 50 | 30 | 20 |
| A-191 | B-1 | C-3 | 50 | 30 | 20 |
| A-192 | B-1 | C-3 | 50 | 30 | 20 |
| A-193 | B-1 | C-3 | 50 | 30 | 20 |
| A-194 | B-1 | C-3 | 50 | 30 | 20 |
| A-195 | B-1 | C-3 | 50 | 30 | 20 |
| A-196 | B-1 | C-3 | 50 | 30 | 20 |
| A-197 | B-1 | C-3 | 50 | 30 | 20 |
| A-198 | B-1 | C-3 | 50 | 30 | 20 |
| A-199 | B-1 | C-3 | 50 | 30 | 20 |
| A-200 | B-1 | C-3 | 50 | 30 | 20 |
| A-201 | B-1 | C-3 | 50 | 30 | 20 |
| A-202 | B-1 | C-3 | 50 | 30 | 20 |
| A-203 | B-1 | C-3 | 50 | 30 | 20 |
| A-204 | B-1 | C-3 | 50 | 30 | 20 |
| A-205 | B-1 | C-3 | 50 | 30 | 20 |
| A-206 | B-1 | C-3 | 50 | 30 | 20 |
| A-207 | B-1 | C-3 | 50 | 30 | 20 |
| A-208 | B-1 | C-3 | 50 | 30 | 20 |
| A-209 | B-1 | C-3 | 50 | 30 | 20 |
| A-210 | B-1 | C-3 | 50 | 30 | 20 |
| A-211 | B-1 | C-3 | 50 | 30 | 20 |
| A-212 | B-1 | C-3 | 50 | 30 | 20 |
| A-213 | B-1 | C-3 | 50 | 30 | 20 |

TABLE 60

| | | | Ratio | | |
|---|---|---|---|---|---|
| | | | of units | | |
| | | | (mol %) | | |
| A | B | C | A | B | C |
| A-220 | B-1 | C-3 | 50 | 30 | 20 |
| A-221 | B-1 | C-3 | 50 | 30 | 20 |
| A-222 | B-1 | C-3 | 50 | 30 | 20 |
| A-223 | B-1 | C-3 | 50 | 30 | 20 |
| A-224 | B-1 | C-3 | 50 | 30 | 20 |
| A-225 | B-1 | C-3 | 50 | 30 | 20 |
| A-226 | B-1 | C-3 | 50 | 30 | 20 |
| A-227 | B-1 | C-3 | 50 | 30 | 20 |
| A-228 | B-1 | C-3 | 50 | 30 | 20 |
| A-229 | B-1 | C-3 | 50 | 30 | 20 |
| A-230 | B-1 | C-3 | 50 | 30 | 20 |
| A-231 | B-1 | C-3 | 50 | 30 | 20 |
| A-232 | B-1 | C-3 | 50 | 30 | 20 |
| A-233 | B-1 | C-3 | 50 | 30 | 20 |
| A-234 | B-1 | C-3 | 50 | 30 | 20 |
| A-235 | B-1 | C-3 | 50 | 30 | 20 |
| A-236 | B-1 | C-3 | 50 | 30 | 20 |
| A-237 | B-1 | C-3 | 50 | 30 | 20 |
| A-238 | B-1 | C-3 | 50 | 30 | 20 |
| A-239 | B-1 | C-3 | 50 | 30 | 20 |
| A-240 | B-1 | C-3 | 50 | 30 | 20 |

TABLE 61

| | | | Ratio of | | |
|---|---|---|---|---|---|
| | | | units | | |
| | | | (mol %) | | |
| A | B | C | A | B | C |
| A-1 | B-1 | C-4 | 50 | 30 | 20 |
| A-2 | B-1 | C-4 | 50 | 30 | 20 |
| A-3 | B-1 | C-4 | 50 | 30 | 20 |
| A-4 | B-1 | C-4 | 50 | 30 | 20 |
| A-5 | B-1 | C-4 | 50 | 30 | 20 |
| A-6 | B-1 | C-4 | 50 | 30 | 20 |
| A-7 | B-1 | C-4 | 50 | 30 | 20 |
| A-8 | B-1 | C-4 | 50 | 30 | 20 |
| A-9 | B-1 | C-4 | 50 | 30 | 20 |
| A-10 | B-1 | C-4 | 50 | 30 | 20 |
| A-11 | B-1 | C-4 | 50 | 30 | 20 |
| A-12 | B-1 | C-4 | 50 | 30 | 20 |
| A-13 | B-1 | C-4 | 50 | 30 | 20 |
| A-14 | B-1 | C-4 | 50 | 30 | 20 |
| A-15 | B-1 | C-4 | 50 | 30 | 20 |
| A-16 | B-1 | C-4 | 50 | 30 | 20 |
| A-17 | B-1 | C-4 | 50 | 30 | 20 |
| A-18 | B-1 | C-4 | 50 | 30 | 20 |
| A-19 | B-1 | C-4 | 50 | 30 | 20 |
| A-20 | B-1 | C-4 | 50 | 30 | 20 |
| A-21 | B-1 | C-4 | 50 | 30 | 20 |
| A-22 | B-1 | C-4 | 50 | 30 | 20 |
| A-23 | B-1 | C-4 | 50 | 30 | 20 |
| A-24 | B-1 | C-4 | 50 | 30 | 20 |
| A-25 | B-1 | C-4 | 50 | 30 | 20 |
| A-26 | B-1 | C-4 | 50 | 30 | 20 |
| A-28 | B-1 | C-4 | 50 | 30 | 20 |
| A-29 | B-1 | C-4 | 50 | 30 | 20 |
| A-30 | B-1 | C-4 | 50 | 30 | 20 |
| A-31 | B-1 | C-4 | 50 | 30 | 20 |
| A-32 | B-1 | C-4 | 50 | 30 | 20 |
| A-33 | B-1 | C-4 | 50 | 30 | 20 |
| A-34 | B-1 | C-4 | 50 | 30 | 20 |
| A-35 | B-1 | C-4 | 50 | 30 | 20 |
| A-36 | B-1 | C-4 | 50 | 30 | 20 |
| A-37 | B-1 | C-4 | 50 | 30 | 20 |
| A-38 | B-1 | C-4 | 50 | 30 | 20 |
| A-39 | B-1 | C-4 | 50 | 30 | 20 |
| A-40 | B-1 | C-4 | 50 | 30 | 20 |
| A-41 | B-1 | C-4 | 50 | 30 | 20 |
| A-42 | B-1 | C-4 | 50 | 30 | 20 |
| A-43 | B-1 | C-4 | 50 | 30 | 20 |
| A-44 | B-1 | C-4 | 50 | 30 | 20 |
| A-45 | B-1 | C-4 | 50 | 30 | 20 |
| A-46 | B-1 | C-4 | 50 | 30 | 20 |
| A-47 | B-1 | C-4 | 50 | 30 | 20 |
| A-49 | B-1 | C-4 | 50 | 30 | 20 |
| A-50 | B-1 | C-4 | 50 | 30 | 20 |
| A-51 | B-1 | C-4 | 50 | 30 | 20 |
| A-52 | B-1 | C-4 | 50 | 30 | 20 |
| A-53 | B-1 | C-4 | 50 | 30 | 20 |
| A-54 | B-1 | C-4 | 50 | 30 | 20 |
| A-55 | B-1 | C-4 | 50 | 30 | 20 |
| A-56 | B-1 | C-4 | 50 | 30 | 20 |
| A-57 | B-1 | C-4 | 50 | 30 | 20 |
| A-58 | B-1 | C-4 | 50 | 30 | 20 |
| A-59 | B-1 | C-4 | 50 | 30 | 20 |
| A-60 | B-1 | C-4 | 50 | 30 | 20 |

TABLE 62

| | | | Ratio of | | |
|---|---|---|---|---|---|
| | | | units | | |
| | | | (mol %) | | |
| A | B | C | A | B | C |
| A-61 | B-1 | C-4 | 50 | 30 | 20 |
| A-62 | B-1 | C-4 | 50 | 30 | 20 |
| A-63 | B-1 | C-4 | 50 | 30 | 20 |
| A-64 | B-1 | C-4 | 50 | 30 | 20 |
| A-65 | B-1 | C-4 | 50 | 30 | 20 |
| A-66 | B-1 | C-4 | 50 | 30 | 20 |
| A-67 | B-1 | C-4 | 50 | 30 | 20 |
| A-68 | B-1 | C-4 | 50 | 30 | 20 |
| A-69 | B-1 | C-4 | 50 | 30 | 20 |
| A-70 | B-1 | C-4 | 50 | 30 | 20 |
| A-71 | B-1 | C-4 | 50 | 30 | 20 |
| A-72 | B-1 | C-4 | 50 | 30 | 20 |
| A-73 | B-1 | C-4 | 50 | 30 | 20 |
| A-74 | B-1 | C-4 | 50 | 30 | 20 |
| A-75 | B-1 | C-4 | 50 | 30 | 20 |
| A-76 | B-1 | C-4 | 50 | 30 | 20 |
| A-77 | B-1 | C-4 | 50 | 30 | 20 |
| A-78 | B-1 | C-4 | 50 | 30 | 20 |
| A-79 | B-1 | C-4 | 50 | 30 | 20 |
| A-80 | B-1 | C-4 | 50 | 30 | 20 |
| A-81 | B-1 | C-4 | 50 | 30 | 20 |
| A-82 | B-1 | C-4 | 50 | 30 | 20 |
| A-83 | B-1 | C-4 | 50 | 30 | 20 |
| A-84 | B-1 | C-4 | 50 | 30 | 20 |
| A-85 | B-1 | C-4 | 50 | 30 | 20 |
| A-86 | B-1 | C-4 | 50 | 30 | 20 |
| A-87 | B-1 | C-4 | 50 | 30 | 20 |
| A-88 | B-1 | C-4 | 50 | 30 | 20 |
| A-89 | B-1 | C-4 | 50 | 30 | 20 |
| A-90 | B-1 | C-4 | 50 | 30 | 20 |
| A-91 | B-1 | C-4 | 50 | 30 | 20 |
| A-92 | B-1 | C-4 | 50 | 30 | 20 |
| A-93 | B-1 | C-4 | 50 | 30 | 20 |
| A-94 | B-1 | C-4 | 50 | 30 | 20 |
| A-95 | B-1 | C-4 | 50 | 30 | 20 |
| A-96 | B-1 | C-4 | 50 | 30 | 20 |
| A-100 | B-1 | C-4 | 50 | 30 | 20 |
| A-101 | B-1 | C-4 | 50 | 30 | 20 |
| A-102 | B-1 | C-4 | 50 | 30 | 20 |
| A-103 | B-1 | C-4 | 50 | 30 | 20 |
| A-104 | B-1 | C-4 | 50 | 30 | 20 |
| A-105 | B-1 | C-4 | 50 | 30 | 20 |
| A-106 | B-1 | C-4 | 50 | 30 | 20 |
| A-107 | B-1 | C-4 | 50 | 30 | 20 |
| A-108 | B-1 | C-4 | 50 | 30 | 20 |
| A-109 | B-1 | C-4 | 50 | 30 | 20 |
| A-110 | B-1 | C-4 | 50 | 30 | 20 |
| A-111 | B-1 | C-4 | 50 | 30 | 20 |
| A-112 | B-1 | C-4 | 50 | 30 | 20 |
| A-113 | B-1 | C-4 | 50 | 30 | 20 |
| A-114 | B-1 | C-4 | 50 | 30 | 20 |
| A-115 | B-1 | C-4 | 50 | 30 | 20 |
| A-116 | B-1 | C-4 | 50 | 30 | 20 |
| A-117 | B-1 | C-4 | 50 | 30 | 20 |
| A-118 | B-1 | C-4 | 50 | 30 | 20 |
| A-119 | B-1 | C-4 | 50 | 30 | 20 |
| A-120 | B-1 | C-4 | 50 | 30 | 20 |
| A-121 | B-1 | C-4 | 50 | 30 | 20 |
| A-122 | B-1 | C-4 | 50 | 30 | 20 |
| A-123 | B-1 | C-4 | 50 | 30 | 20 |

TABLE 63

| | | | Ratio of | | |
|---|---|---|---|---|---|
| | | | units | | |
| | | | (mol %) | | |
| A | B | C | A | B | C |
| A-124 | B-1 | C-4 | 50 | 30 | 20 |
| A-125 | B-1 | C-4 | 50 | 30 | 20 |
| A-126 | B-1 | C-4 | 50 | 30 | 20 |
| A-127 | B-1 | C-4 | 50 | 30 | 20 |
| A-128 | B-1 | C-4 | 50 | 30 | 20 |
| A-129 | B-1 | C-4 | 50 | 30 | 20 |
| A-130 | B-1 | C-4 | 50 | 30 | 20 |
| A-131 | B-1 | C-4 | 50 | 30 | 20 |
| A-132 | B-1 | C-4 | 50 | 30 | 20 |
| A-133 | B-1 | C-4 | 50 | 30 | 20 |
| A-134 | B-1 | C-4 | 50 | 30 | 20 |
| A-135 | B-1 | C-4 | 50 | 30 | 20 |
| A-136 | B-1 | C-4 | 50 | 30 | 20 |
| A-137 | B-1 | C-4 | 50 | 30 | 20 |
| A-138 | B-1 | C-4 | 50 | 30 | 20 |
| A-139 | B-1 | C-4 | 50 | 30 | 20 |
| A-140 | B-1 | C-4 | 50 | 30 | 20 |
| A-141 | B-1 | C-4 | 50 | 30 | 20 |
| A-142 | B-1 | C-4 | 50 | 30 | 20 |
| A-143 | B-1 | C-4 | 50 | 30 | 20 |
| A-144 | B-1 | C-4 | 50 | 30 | 20 |
| A-145 | B-1 | C-4 | 50 | 30 | 20 |
| A-146 | B-1 | C-4 | 50 | 30 | 20 |
| A-147 | B-1 | C-4 | 50 | 30 | 20 |
| A-160 | B-1 | C-4 | 50 | 30 | 20 |
| A-161 | B-1 | C-4 | 50 | 30 | 20 |
| A-162 | B-1 | C-4 | 50 | 30 | 20 |
| A-163 | B-1 | C-4 | 50 | 30 | 20 |
| A-164 | B-1 | C-4 | 50 | 30 | 20 |
| A-165 | B-1 | C-4 | 50 | 30 | 20 |
| A-166 | B-1 | C-4 | 50 | 30 | 20 |
| A-167 | B-1 | C-4 | 50 | 30 | 20 |
| A-168 | B-1 | C-4 | 50 | 30 | 20 |
| A-169 | B-1 | C-4 | 50 | 30 | 20 |
| A-170 | B-1 | C-4 | 50 | 30 | 20 |
| A-171 | B-1 | C-4 | 50 | 30 | 20 |
| A-172 | B-1 | C-4 | 50 | 30 | 20 |
| A-173 | B-1 | C-4 | 50 | 30 | 20 |
| A-174 | B-1 | C-4 | 50 | 30 | 20 |
| A-175 | B-1 | C-4 | 50 | 30 | 20 |
| A-176 | B-1 | C-4 | 50 | 30 | 20 |
| A-177 | B-1 | C-4 | 50 | 30 | 20 |
| A-178 | B-1 | C-4 | 50 | 30 | 20 |
| A-179 | B-1 | C-4 | 50 | 30 | 20 |
| A-180 | B-1 | C-4 | 50 | 30 | 20 |
| A-181 | B-1 | C-4 | 50 | 30 | 20 |
| A-182 | B-1 | C-4 | 50 | 30 | 20 |
| A-183 | B-1 | C-4 | 50 | 30 | 20 |
| A-190 | B-1 | C-4 | 50 | 30 | 20 |
| A-191 | B-1 | C-4 | 50 | 30 | 20 |
| A-192 | B-1 | C-4 | 50 | 30 | 20 |
| A-193 | B-1 | C-4 | 50 | 30 | 20 |
| A-194 | B-1 | C-4 | 50 | 30 | 20 |
| A-195 | B-1 | C-4 | 50 | 30 | 20 |
| A-196 | B-1 | C-4 | 50 | 30 | 20 |
| A-197 | B-1 | C-4 | 50 | 30 | 20 |
| A-198 | B-1 | C-4 | 50 | 30 | 20 |
| A-199 | B-1 | C-4 | 50 | 30 | 20 |
| A-200 | B-1 | C-4 | 50 | 30 | 20 |
| A-201 | B-1 | C-4 | 50 | 30 | 20 |
| A-202 | B-1 | C-4 | 50 | 30 | 20 |
| A-203 | B-1 | C-4 | 50 | 30 | 20 |
| A-204 | B-1 | C-4 | 50 | 30 | 20 |
| A-205 | B-1 | C-4 | 50 | 30 | 20 |
| A-206 | B-1 | C-4 | 50 | 30 | 20 |
| A-207 | B-1 | C-4 | 50 | 30 | 20 |
| A-208 | B-1 | C-4 | 50 | 30 | 20 |
| A-209 | B-1 | C-4 | 50 | 30 | 20 |
| A-210 | B-1 | C-4 | 50 | 30 | 20 |
| A-211 | B-1 | C-4 | 50 | 30 | 20 |
| A-212 | B-1 | C-4 | 50 | 30 | 20 |
| A-213 | B-1 | C-4 | 50 | 30 | 20 |

TABLE 64

| | | | Ratio of | | |
|---|---|---|---|---|---|
| | | | units | | |
| | | | (mol %) | | |
| A | B | C | A | B | C |
| A-220 | B-1 | C-4 | 50 | 30 | 20 |
| A-221 | B-1 | C-4 | 50 | 30 | 20 |
| A-222 | B-1 | C-4 | 50 | 30 | 20 |
| A-223 | B-1 | C-4 | 50 | 30 | 20 |
| A-224 | B-1 | C-4 | 50 | 30 | 20 |
| A-225 | B-1 | C-4 | 50 | 30 | 20 |
| A-226 | B-1 | C-4 | 50 | 30 | 20 |
| A-227 | B-1 | C-4 | 50 | 30 | 20 |
| A-228 | B-1 | C-4 | 50 | 30 | 20 |
| A-229 | B-1 | C-4 | 50 | 30 | 20 |
| A-230 | B-1 | C-4 | 50 | 30 | 20 |
| A-231 | B-1 | C-4 | 50 | 30 | 20 |
| A-232 | B-1 | C-4 | 50 | 30 | 20 |
| A-233 | B-1 | C-4 | 50 | 30 | 20 |
| A-234 | B-1 | C-4 | 50 | 30 | 20 |
| A-235 | B-1 | C-4 | 50 | 30 | 20 |
| A-236 | B-1 | C-4 | 50 | 30 | 20 |
| A-237 | B-1 | C-4 | 50 | 30 | 20 |
| A-238 | B-1 | C-4 | 50 | 30 | 20 |
| A-239 | B-1 | C-4 | 50 | 30 | 20 |
| A-240 | B-1 | C-4 | 50 | 30 | 20 |

TABLE 65

| | | | Ratio of units (mol %) | | |
|---|---|---|---|---|---|
| A | B | C | A | B | C |
| A-1 | B-2 | C-1 | 50 | 47 | 3 |
| A-2 | B-2 | C-1 | 50 | 47 | 3 |
| A-3 | B-2 | C-1 | 50 | 47 | 3 |
| A-4 | B-2 | C-1 | 50 | 47 | 3 |
| A-5 | B-2 | C-1 | 50 | 47 | 3 |
| A-6 | B-2 | C-1 | 50 | 47 | 3 |
| A-7 | B-2 | C-1 | 50 | 47 | 3 |
| A-8 | B-2 | C-1 | 50 | 47 | 3 |
| A-9 | B-2 | C-1 | 50 | 47 | 3 |
| A-10 | B-2 | C-1 | 50 | 47 | 3 |
| A-11 | B-2 | C-1 | 50 | 47 | 3 |
| A-12 | B-2 | C-1 | 50 | 47 | 3 |
| A-13 | B-2 | C-1 | 50 | 47 | 3 |
| A-14 | B-2 | C-1 | 50 | 47 | 3 |
| A-15 | B-2 | C-1 | 50 | 47 | 3 |
| A-16 | B-2 | C-1 | 50 | 47 | 3 |
| A-17 | B-2 | C-1 | 50 | 47 | 3 |
| A-18 | B-2 | C-1 | 50 | 47 | 3 |
| A-19 | B-2 | C-1 | 50 | 47 | 3 |
| A-20 | B-2 | C-1 | 50 | 47 | 3 |
| A-21 | B-2 | C-1 | 50 | 47 | 3 |
| A-22 | B-2 | C-1 | 50 | 47 | 3 |
| A-23 | B-2 | C-1 | 50 | 47 | 3 |
| A-24 | B-2 | C-1 | 50 | 47 | 3 |
| A-25 | B-2 | C-1 | 50 | 47 | 3 |
| A-26 | B-2 | C-1 | 50 | 47 | 3 |
| A-28 | B-2 | C-1 | 50 | 47 | 3 |
| A-29 | B-2 | C-1 | 50 | 47 | 3 |
| A-30 | B-2 | C-1 | 50 | 47 | 3 |
| A-31 | B-2 | C-1 | 50 | 47 | 3 |
| A-32 | B-2 | C-1 | 50 | 47 | 3 |
| A-33 | B-2 | C-1 | 50 | 47 | 3 |
| A-34 | B-2 | C-1 | 50 | 47 | 3 |
| A-35 | B-2 | C-1 | 50 | 47 | 3 |
| A-36 | B-2 | C-1 | 50 | 47 | 3 |
| A-37 | B-2 | C-1 | 50 | 47 | 3 |
| A-38 | B-2 | C-1 | 50 | 47 | 3 |
| A-39 | B-2 | C-1 | 50 | 47 | 3 |
| A-40 | B-2 | C-1 | 50 | 47 | 3 |
| A-41 | B-2 | C-1 | 50 | 47 | 3 |
| A-42 | B-2 | C-1 | 50 | 47 | 3 |
| A-43 | B-2 | C-1 | 50 | 47 | 3 |
| A-44 | B-2 | C-1 | 50 | 47 | 3 |
| A-45 | B-2 | C-1 | 50 | 47 | 3 |
| A-46 | B-2 | C-1 | 50 | 47 | 3 |
| A-47 | B-2 | C-1 | 50 | 47 | 3 |
| A-49 | B-2 | C-1 | 50 | 47 | 3 |
| A-50 | B-2 | C-1 | 50 | 47 | 3 |
| A-51 | B-2 | C-1 | 50 | 47 | 3 |
| A-52 | B-2 | C-1 | 50 | 47 | 3 |
| A-53 | B-2 | C-1 | 50 | 47 | 3 |
| A-54 | B-2 | C-1 | 50 | 47 | 3 |
| A-55 | B-2 | C-1 | 50 | 47 | 3 |
| A-56 | B-2 | C-1 | 50 | 47 | 3 |
| A-57 | B-2 | C-1 | 50 | 47 | 3 |
| A-58 | B-2 | C-1 | 50 | 47 | 3 |
| A-59 | B-2 | C-1 | 50 | 47 | 3 |
| A-60 | B-2 | C-1 | 50 | 47 | 3 |

TABLE 66

| | | | Ratio of units (mol %) | | |
|---|---|---|---|---|---|
| A | B | C | A | B | C |
| A-61 | B-2 | C-1 | 50 | 47 | 3 |
| A-62 | B-2 | C-1 | 50 | 47 | 3 |
| A-63 | B-2 | C-1 | 50 | 47 | 3 |
| A-64 | B-2 | C-1 | 50 | 47 | 3 |
| A-65 | B-2 | C-1 | 50 | 47 | 3 |
| A-66 | B-2 | C-1 | 50 | 47 | 3 |
| A-67 | B-2 | C-1 | 50 | 47 | 3 |
| A-68 | B-2 | C-1 | 50 | 47 | 3 |
| A-69 | B-2 | C-1 | 50 | 47 | 3 |
| A-70 | B-2 | C-1 | 50 | 47 | 3 |
| A-71 | B-2 | C-1 | 50 | 47 | 3 |
| A-72 | B-2 | C-1 | 50 | 47 | 3 |
| A-73 | B-2 | C-1 | 50 | 47 | 3 |
| A-74 | B-2 | C-1 | 50 | 47 | 3 |
| A-75 | B-2 | C-1 | 50 | 47 | 3 |
| A-76 | B-2 | C-1 | 50 | 47 | 3 |
| A-77 | B-2 | C-1 | 50 | 47 | 3 |
| A-78 | B-2 | C-1 | 50 | 47 | 3 |
| A-79 | B-2 | C-1 | 50 | 47 | 3 |
| A-80 | B-2 | C-1 | 50 | 47 | 3 |
| A-81 | B-2 | C-1 | 50 | 47 | 3 |
| A-82 | B-2 | C-1 | 50 | 47 | 3 |
| A-83 | B-2 | C-1 | 50 | 47 | 3 |
| A-84 | B-2 | C-1 | 50 | 47 | 3 |
| A-85 | B-2 | C-1 | 50 | 47 | 3 |
| A-86 | B-2 | C-1 | 50 | 47 | 3 |
| A-87 | B-2 | C-1 | 50 | 47 | 3 |
| A-88 | B-2 | C-1 | 50 | 47 | 3 |
| A-89 | B-2 | C-1 | 50 | 47 | 3 |
| A-90 | B-2 | C-1 | 50 | 47 | 3 |
| A-91 | B-2 | C-1 | 50 | 47 | 3 |
| A-92 | B-2 | C-1 | 50 | 47 | 3 |
| A-93 | B-2 | C-1 | 50 | 47 | 3 |
| A-94 | B-2 | C-1 | 50 | 47 | 3 |
| A-95 | B-2 | C-1 | 50 | 47 | 3 |
| A-96 | B-2 | C-1 | 50 | 47 | 3 |
| A-100 | B-2 | C-1 | 50 | 47 | 3 |
| A-101 | B-2 | C-1 | 50 | 47 | 3 |
| A-102 | B-2 | C-1 | 50 | 47 | 3 |
| A-103 | B-2 | C-1 | 50 | 47 | 3 |
| A-104 | B-2 | C-1 | 50 | 47 | 3 |
| A-105 | B-2 | C-1 | 50 | 47 | 3 |
| A-106 | B-2 | C-1 | 50 | 47 | 3 |
| A-107 | B-2 | C-1 | 50 | 47 | 3 |
| A-108 | B-2 | C-1 | 50 | 47 | 3 |
| A-109 | B-2 | C-1 | 50 | 47 | 3 |
| A-110 | B-2 | C-1 | 50 | 47 | 3 |
| A-111 | B-2 | C-1 | 50 | 47 | 3 |
| A-112 | B-2 | C-1 | 50 | 47 | 3 |
| A-113 | B-2 | C-1 | 50 | 47 | 3 |
| A-114 | B-2 | C-1 | 50 | 47 | 3 |
| A-115 | B-2 | C-1 | 50 | 47 | 3 |
| A-116 | B-2 | C-1 | 50 | 47 | 3 |
| A-117 | B-2 | C-1 | 50 | 47 | 3 |
| A-118 | B-2 | C-1 | 50 | 47 | 3 |
| A-119 | B-2 | C-1 | 50 | 47 | 3 |
| A-120 | B-2 | C-1 | 50 | 47 | 3 |
| A-121 | B-2 | C-1 | 50 | 47 | 3 |
| A-122 | B-2 | C-1 | 50 | 47 | 3 |
| A-123 | B-2 | C-1 | 50 | 47 | 3 |

TABLE 67

| | | | Ratio of units (mol %) | | |
|---|---|---|---|---|---|
| A | B | C | A | B | C |
| A-124 | B-2 | C-1 | 50 | 47 | 3 |
| A-125 | B-2 | C-1 | 50 | 47 | 3 |
| A-126 | B-2 | C-1 | 50 | 47 | 3 |
| A-127 | B-2 | C-1 | 50 | 47 | 3 |
| A-128 | B-2 | C-1 | 50 | 47 | 3 |
| A-129 | B-2 | C-1 | 50 | 47 | 3 |
| A-130 | B-2 | C-1 | 50 | 47 | 3 |
| A-131 | B-2 | C-1 | 50 | 47 | 3 |
| A-132 | B-2 | C-1 | 50 | 47 | 3 |
| A-133 | B-2 | C-1 | 50 | 47 | 3 |
| A-134 | B-2 | C-1 | 50 | 47 | 3 |
| A-135 | B-2 | C-1 | 50 | 47 | 3 |
| A-136 | B-2 | C-1 | 50 | 47 | 3 |
| A-137 | B-2 | C-1 | 50 | 47 | 3 |
| A-138 | B-2 | C-1 | 50 | 47 | 3 |
| A-139 | B-2 | C-1 | 50 | 47 | 3 |
| A-140 | B-2 | C-1 | 50 | 47 | 3 |
| A-141 | B-2 | C-1 | 50 | 47 | 3 |
| A-142 | B-2 | C-1 | 50 | 47 | 3 |
| A-143 | B-2 | C-1 | 50 | 47 | 3 |
| A-144 | B-2 | C-1 | 50 | 47 | 3 |
| A-145 | B-2 | C-1 | 50 | 47 | 3 |
| A-146 | B-2 | C-1 | 50 | 47 | 3 |
| A-147 | B-2 | C-1 | 50 | 47 | 3 |
| A-160 | B-2 | C-1 | 50 | 47 | 3 |
| A-161 | B-2 | C-1 | 50 | 47 | 3 |
| A-162 | B-2 | C-1 | 50 | 47 | 3 |
| A-163 | B-2 | C-1 | 50 | 47 | 3 |
| A-164 | B-2 | C-1 | 50 | 47 | 3 |
| A-165 | B-2 | C-1 | 50 | 47 | 3 |
| A-166 | B-2 | C-1 | 50 | 47 | 3 |
| A-167 | B-2 | C-1 | 50 | 47 | 3 |
| A-168 | B-2 | C-1 | 50 | 47 | 3 |
| A-169 | B-2 | C-1 | 50 | 47 | 3 |
| A-170 | B-2 | C-1 | 50 | 47 | 3 |
| A-171 | B-2 | C-1 | 50 | 47 | 3 |
| A-172 | B-2 | C-1 | 50 | 47 | 3 |
| A-173 | B-2 | C-1 | 50 | 47 | 3 |
| A-174 | B-2 | C-1 | 50 | 47 | 3 |
| A-175 | B-2 | C-1 | 50 | 47 | 3 |
| A-176 | B-2 | C-1 | 50 | 47 | 3 |
| A-177 | B-2 | C-1 | 50 | 47 | 3 |
| A-178 | B-2 | C-1 | 50 | 47 | 3 |
| A-179 | B-2 | C-1 | 50 | 47 | 3 |
| A-180 | B-2 | C-1 | 50 | 47 | 3 |
| A-181 | B-2 | C-1 | 50 | 47 | 3 |
| A-182 | B-2 | C-1 | 50 | 47 | 3 |
| A-183 | B-2 | C-1 | 50 | 47 | 3 |
| A-190 | B-2 | C-1 | 50 | 47 | 3 |
| A-191 | B-2 | C-1 | 50 | 47 | 3 |
| A-192 | B-2 | C-1 | 50 | 47 | 3 |
| A-193 | B-2 | C-1 | 50 | 47 | 3 |
| A-194 | B-2 | C-1 | 50 | 47 | 3 |
| A-195 | B-2 | C-1 | 50 | 47 | 3 |
| A-196 | B-2 | C-1 | 50 | 47 | 3 |
| A-197 | B-2 | C-1 | 50 | 47 | 3 |
| A-198 | B-2 | C-1 | 50 | 47 | 3 |
| A-199 | B-2 | C-1 | 50 | 47 | 3 |
| A-200 | B-2 | C-1 | 50 | 47 | 3 |
| A-201 | B-2 | C-1 | 50 | 47 | 3 |
| A-202 | B-2 | C-1 | 50 | 47 | 3 |
| A-203 | B-2 | C-1 | 50 | 47 | 3 |
| A-204 | B-2 | C-1 | 50 | 47 | 3 |
| A-205 | B-2 | C-1 | 50 | 47 | 3 |
| A-206 | B-2 | C-1 | 50 | 47 | 3 |
| A-207 | B-2 | C-1 | 50 | 47 | 3 |
| A-208 | B-2 | C-1 | 50 | 47 | 3 |
| A-209 | B-2 | C-1 | 50 | 47 | 3 |
| A-210 | B-2 | C-1 | 50 | 47 | 3 |
| A-211 | B-2 | C-1 | 50 | 47 | 3 |
| A-212 | B-2 | C-1 | 50 | 47 | 3 |
| A-213 | B-2 | C-1 | 50 | 47 | 3 |

TABLE 68

| | | | Ratio of units (mol %) | | |
|---|---|---|---|---|---|
| A | B | C | A | B | C |
| A-220 | B-2 | C-1 | 50 | 47 | 3 |
| A-221 | B-2 | C-1 | 50 | 47 | 3 |
| A-222 | B-2 | C-1 | 50 | 47 | 3 |
| A-223 | B-2 | C-1 | 50 | 47 | 3 |
| A-224 | B-2 | C-1 | 50 | 47 | 3 |
| A-225 | B-2 | C-1 | 50 | 47 | 3 |
| A-226 | B-2 | C-1 | 50 | 47 | 3 |
| A-227 | B-2 | C-1 | 50 | 47 | 3 |
| A-228 | B-2 | C-1 | 50 | 47 | 3 |
| A-229 | B-2 | C-1 | 50 | 47 | 3 |
| A-230 | B-2 | C-1 | 50 | 47 | 3 |
| A-231 | B-2 | C-1 | 50 | 47 | 3 |
| A-232 | B-2 | C-1 | 50 | 47 | 3 |
| A-233 | B-2 | C-1 | 50 | 47 | 3 |
| A-234 | B-2 | C-1 | 50 | 47 | 3 |
| A-235 | B-2 | C-1 | 50 | 47 | 3 |
| A-236 | B-2 | C-1 | 50 | 47 | 3 |
| A-237 | B-2 | C-1 | 50 | 47 | 3 |
| A-238 | B-2 | C-1 | 50 | 47 | 3 |
| A-239 | B-2 | C-1 | 50 | 47 | 3 |
| A-240 | B-2 | C-1 | 50 | 47 | 3 |

TABLE 69

| | | | Ratio of units (mol %) | | |
|---|---|---|---|---|---|
| A | B | C | A | B | C |
| A-1 | B-2 | C-2 | 50 | 47 | 3 |
| A-2 | B-2 | C-2 | 50 | 47 | 3 |
| A-3 | B-2 | C-2 | 50 | 47 | 3 |
| A-4 | B-2 | C-2 | 50 | 47 | 3 |
| A-5 | B-2 | C-2 | 50 | 47 | 3 |
| A-6 | B-2 | C-2 | 50 | 47 | 3 |
| A-7 | B-2 | C-2 | 50 | 47 | 3 |
| A-8 | B-2 | C-2 | 50 | 47 | 3 |
| A-9 | B-2 | C-2 | 50 | 47 | 3 |
| A-10 | B-2 | C-2 | 50 | 47 | 3 |
| A-11 | B-2 | C-2 | 50 | 47 | 3 |
| A-12 | B-2 | C-2 | 50 | 47 | 3 |
| A-13 | B-2 | C-2 | 50 | 47 | 3 |
| A-14 | B-2 | C-2 | 50 | 47 | 3 |
| A-15 | B-2 | C-2 | 50 | 47 | 3 |
| A-16 | B-2 | C-2 | 50 | 47 | 3 |
| A-17 | B-2 | C-2 | 50 | 47 | 3 |
| A-18 | B-2 | C-2 | 50 | 47 | 3 |
| A-19 | B-2 | C-2 | 50 | 47 | 3 |
| A-20 | B-2 | C-2 | 50 | 47 | 3 |
| A-21 | B-2 | C-2 | 50 | 47 | 3 |
| A-22 | B-2 | C-2 | 50 | 47 | 3 |
| A-23 | B-2 | C-2 | 50 | 47 | 3 |
| A-24 | B-2 | C-2 | 50 | 47 | 3 |
| A-25 | B-2 | C-2 | 50 | 47 | 3 |
| A-26 | B-2 | C-2 | 50 | 47 | 3 |
| A-28 | B-2 | C-2 | 50 | 47 | 3 |
| A-29 | B-2 | C-2 | 50 | 47 | 3 |
| A-30 | B-2 | C-2 | 50 | 47 | 3 |
| A-31 | B-2 | C-2 | 50 | 47 | 3 |
| A-32 | B-2 | C-2 | 50 | 47 | 3 |
| A-33 | B-2 | C-2 | 50 | 47 | 3 |
| A-34 | B-2 | C-2 | 50 | 47 | 3 |
| A-35 | B-2 | C-2 | 50 | 47 | 3 |
| A-36 | B-2 | C-2 | 50 | 47 | 3 |
| A-37 | B-2 | C-2 | 50 | 47 | 3 |
| A-38 | B-2 | C-2 | 50 | 47 | 3 |
| A-39 | B-2 | C-2 | 50 | 47 | 3 |
| A-40 | B-2 | C-2 | 50 | 47 | 3 |
| A-41 | B-2 | C-2 | 50 | 47 | 3 |
| A-42 | B-2 | C-2 | 50 | 47 | 3 |
| A-43 | B-2 | C-2 | 50 | 47 | 3 |
| A-44 | B-2 | C-2 | 50 | 47 | 3 |
| A-45 | B-2 | C-2 | 50 | 47 | 3 |
| A-46 | B-2 | C-2 | 50 | 47 | 3 |
| A-47 | B-2 | C-2 | 50 | 47 | 3 |
| A-49 | B-2 | C-2 | 50 | 47 | 3 |
| A-50 | B-2 | C-2 | 50 | 47 | 3 |
| A-51 | B-2 | C-2 | 50 | 47 | 3 |
| A-52 | B-2 | C-2 | 50 | 47 | 3 |
| A-53 | B-2 | C-2 | 50 | 47 | 3 |
| A-54 | B-2 | C-2 | 50 | 47 | 3 |
| A-55 | B-2 | C-2 | 50 | 47 | 3 |
| A-56 | B-2 | C-2 | 50 | 47 | 3 |
| A-57 | B-2 | C-2 | 50 | 47 | 3 |
| A-58 | B-2 | C-2 | 50 | 47 | 3 |
| A-59 | B-2 | C-2 | 50 | 47 | 3 |
| A-60 | B-2 | C-2 | 50 | 47 | 3 |

TABLE 70

| | | | Ratio of units (mol %) | | |
|---|---|---|---|---|---|
| A | B | C | A | B | C |
| A-61 | B-2 | C-2 | 50 | 47 | 3 |
| A-62 | B-2 | C-2 | 50 | 47 | 3 |
| A-63 | B-2 | C-2 | 50 | 47 | 3 |
| A-64 | B-2 | C-2 | 50 | 47 | 3 |
| A-65 | B-2 | C-2 | 50 | 47 | 3 |
| A-66 | B-2 | C-2 | 50 | 47 | 3 |
| A-67 | B-2 | C-2 | 50 | 47 | 3 |
| A-68 | B-2 | C-2 | 50 | 47 | 3 |
| A-69 | B-2 | C-2 | 50 | 47 | 3 |
| A-70 | B-2 | C-2 | 50 | 47 | 3 |
| A-71 | B-2 | C-2 | 50 | 47 | 3 |
| A-72 | B-2 | C-2 | 50 | 47 | 3 |
| A-73 | B-2 | C-2 | 50 | 47 | 3 |
| A-74 | B-2 | C-2 | 50 | 47 | 3 |
| A-75 | B-2 | C-2 | 50 | 47 | 3 |
| A-76 | B-2 | C-2 | 50 | 47 | 3 |
| A-77 | B-2 | C-2 | 50 | 47 | 3 |
| A-78 | B-2 | C-2 | 50 | 47 | 3 |
| A-79 | B-2 | C-2 | 50 | 47 | 3 |
| A-80 | B-2 | C-2 | 50 | 47 | 3 |
| A-81 | B-2 | C-2 | 50 | 47 | 3 |
| A-82 | B-2 | C-2 | 50 | 47 | 3 |
| A-83 | B-2 | C-2 | 50 | 47 | 3 |
| A-84 | B-2 | C-2 | 50 | 47 | 3 |
| A-85 | B-2 | C-2 | 50 | 47 | 3 |
| A-86 | B-2 | C-2 | 50 | 47 | 3 |
| A-87 | B-2 | C-2 | 50 | 47 | 3 |
| A-88 | B-2 | C-2 | 50 | 47 | 3 |
| A-89 | B-2 | C-2 | 50 | 47 | 3 |
| A-90 | B-2 | C-2 | 50 | 47 | 3 |
| A-91 | B-2 | C-2 | 50 | 47 | 3 |
| A-92 | B-2 | C-2 | 50 | 47 | 3 |
| A-93 | B-2 | C-2 | 50 | 47 | 3 |
| A-94 | B-2 | C-2 | 50 | 47 | 3 |
| A-95 | B-2 | C-2 | 50 | 47 | 3 |
| A-96 | B-2 | C-2 | 50 | 47 | 3 |
| A-100 | B-2 | C-2 | 50 | 47 | 3 |
| A-101 | B-2 | C-2 | 50 | 47 | 3 |
| A-102 | B-2 | C-2 | 50 | 47 | 3 |
| A-103 | B-2 | C-2 | 50 | 47 | 3 |
| A-104 | B-2 | C-2 | 50 | 47 | 3 |
| A-105 | B-2 | C-2 | 50 | 47 | 3 |
| A-106 | B-2 | C-2 | 50 | 47 | 3 |
| A-107 | B-2 | C-2 | 50 | 47 | 3 |
| A-108 | B-2 | C-2 | 50 | 47 | 3 |
| A-109 | B-2 | C-2 | 50 | 47 | 3 |
| A-110 | B-2 | C-2 | 50 | 47 | 3 |
| A-111 | B-2 | C-2 | 50 | 47 | 3 |
| A-112 | B-2 | C-2 | 50 | 47 | 3 |
| A-113 | B-2 | C-2 | 50 | 47 | 3 |
| A-114 | B-2 | C-2 | 50 | 47 | 3 |
| A-115 | B-2 | C-2 | 50 | 47 | 3 |
| A-116 | B-2 | C-2 | 50 | 47 | 3 |
| A-117 | B-2 | C-2 | 50 | 47 | 3 |
| A-118 | B-2 | C-2 | 50 | 47 | 3 |
| A-119 | B-2 | C-2 | 50 | 47 | 3 |
| A-120 | B-2 | C-2 | 50 | 47 | 3 |
| A-121 | B-2 | C-2 | 50 | 47 | 3 |
| A-122 | B-2 | C-2 | 50 | 47 | 3 |
| A-123 | B-2 | C-2 | 50 | 47 | 3 |

TABLE 71

| | | | Ratio of units (mol %) | | |
|---|---|---|---|---|---|
| A | B | C | A | B | C |
| A-124 | B-2 | C-2 | 50 | 47 | 3 |
| A-125 | B-2 | C-2 | 50 | 47 | 3 |
| A-126 | B-2 | C-2 | 50 | 47 | 3 |
| A-127 | B-2 | C-2 | 50 | 47 | 3 |
| A-128 | B-2 | C-2 | 50 | 47 | 3 |
| A-129 | B-2 | C-2 | 50 | 47 | 3 |
| A-130 | B-2 | C-2 | 50 | 47 | 3 |
| A-131 | B-2 | C-2 | 50 | 47 | 3 |
| A-132 | B-2 | C-2 | 50 | 47 | 3 |
| A-133 | B-2 | C-2 | 50 | 47 | 3 |
| A-134 | B-2 | C-2 | 50 | 47 | 3 |
| A-135 | B-2 | C-2 | 50 | 47 | 3 |
| A-136 | B-2 | C-2 | 50 | 47 | 3 |
| A-137 | B-2 | C-2 | 50 | 47 | 3 |
| A-138 | B-2 | C-2 | 50 | 47 | 3 |
| A-139 | B-2 | C-2 | 50 | 47 | 3 |
| A-140 | B-2 | C-2 | 50 | 47 | 3 |
| A-141 | B-2 | C-2 | 50 | 47 | 3 |
| A-142 | B-2 | C-2 | 50 | 47 | 3 |
| A-143 | B-2 | C-2 | 50 | 47 | 3 |
| A-144 | B-2 | C-2 | 50 | 47 | 3 |
| A-145 | B-2 | C-2 | 50 | 47 | 3 |
| A-146 | B-2 | C-2 | 50 | 47 | 3 |
| A-147 | B-2 | C-2 | 50 | 47 | 3 |
| A-160 | B-2 | C-2 | 50 | 47 | 3 |
| A-161 | B-2 | C-2 | 50 | 47 | 3 |
| A-162 | B-2 | C-2 | 50 | 47 | 3 |
| A-163 | B-2 | C-2 | 50 | 47 | 3 |
| A-164 | B-2 | C-2 | 50 | 47 | 3 |
| A-165 | B-2 | C-2 | 50 | 47 | 3 |
| A-166 | B-2 | C-2 | 50 | 47 | 3 |
| A-167 | B-2 | C-2 | 50 | 47 | 3 |
| A-168 | B-2 | C-2 | 50 | 47 | 3 |
| A-169 | B-2 | C-2 | 50 | 47 | 3 |
| A-170 | B-2 | C-2 | 50 | 47 | 3 |
| A-171 | B-2 | C-2 | 50 | 47 | 3 |
| A-172 | B-2 | C-2 | 50 | 47 | 3 |
| A-173 | B-2 | C-2 | 50 | 47 | 3 |
| A-174 | B-2 | C-2 | 50 | 47 | 3 |
| A-175 | B-2 | C-2 | 50 | 47 | 3 |
| A-176 | B-2 | C-2 | 50 | 47 | 3 |
| A-177 | B-2 | C-2 | 50 | 47 | 3 |
| A-178 | B-2 | C-2 | 50 | 47 | 3 |
| A-179 | B-2 | C-2 | 50 | 47 | 3 |
| A-180 | B-2 | C-2 | 50 | 47 | 3 |
| A-181 | B-2 | C-2 | 50 | 47 | 3 |
| A-182 | B-2 | C-2 | 50 | 47 | 3 |
| A-183 | B-2 | C-2 | 50 | 47 | 3 |
| A-190 | B-2 | C-2 | 50 | 47 | 3 |
| A-191 | B-2 | C-2 | 50 | 47 | 3 |
| A-192 | B-2 | C-2 | 50 | 47 | 3 |
| A-193 | B-2 | C-2 | 50 | 47 | 3 |
| A-194 | B-2 | C-2 | 50 | 47 | 3 |
| A-195 | B-2 | C-2 | 50 | 47 | 3 |
| A-196 | B-2 | C-2 | 50 | 47 | 3 |
| A-197 | B-2 | C-2 | 50 | 47 | 3 |
| A-198 | B-2 | C-2 | 50 | 47 | 3 |
| A-199 | B-2 | C-2 | 50 | 47 | 3 |
| A-200 | B-2 | C-2 | 50 | 47 | 3 |
| A-201 | B-2 | C-2 | 50 | 47 | 3 |
| A-202 | B-2 | C-2 | 50 | 47 | 3 |
| A-203 | B-2 | C-2 | 50 | 47 | 3 |
| A-204 | B-2 | C-2 | 50 | 47 | 3 |
| A-205 | B-2 | C-2 | 50 | 47 | 3 |
| A-206 | B-2 | C-2 | 50 | 47 | 3 |
| A-207 | B-2 | C-2 | 50 | 47 | 3 |
| A-208 | B-2 | C-2 | 50 | 47 | 3 |
| A-209 | B-2 | C-2 | 50 | 47 | 3 |
| A-210 | B-2 | C-2 | 50 | 47 | 3 |
| A-211 | B-2 | C-2 | 50 | 47 | 3 |
| A-212 | B-2 | C-2 | 50 | 47 | 3 |
| A-213 | B-2 | C-2 | 50 | 47 | 3 |

TABLE 72

| | | | Ratio of units (mol %) | | |
|---|---|---|---|---|---|
| A | B | C | A | B | C |
| A-220 | B-2 | C-2 | 50 | 47 | 3 |
| A-221 | B-2 | C-2 | 50 | 47 | 3 |
| A-222 | B-2 | C-2 | 50 | 47 | 3 |
| A-223 | B-2 | C-2 | 50 | 47 | 3 |
| A-224 | B-2 | C-2 | 50 | 47 | 3 |
| A-225 | B-2 | C-2 | 50 | 47 | 3 |
| A-226 | B-2 | C-2 | 50 | 47 | 3 |
| A-227 | B-2 | C-2 | 50 | 47 | 3 |
| A-228 | B-2 | C-2 | 50 | 47 | 3 |
| A-229 | B-2 | C-2 | 50 | 47 | 3 |
| A-230 | B-2 | C-2 | 50 | 47 | 3 |
| A-231 | B-2 | C-2 | 50 | 47 | 3 |
| A-232 | B-2 | C-2 | 50 | 47 | 3 |
| A-233 | B-2 | C-2 | 50 | 47 | 3 |
| A-234 | B-2 | C-2 | 50 | 47 | 3 |
| A-235 | B-2 | C-2 | 50 | 47 | 3 |
| A-236 | B-2 | C-2 | 50 | 47 | 3 |
| A-237 | B-2 | C-2 | 50 | 47 | 3 |
| A-238 | B-2 | C-2 | 50 | 47 | 3 |
| A-239 | B-2 | C-2 | 50 | 47 | 3 |
| A-240 | B-2 | C-2 | 50 | 47 | 3 |

TABLE 73

| | | | Ratio of units (mol %) | | |
|---|---|---|---|---|---|
| A | B | C | A | B | C |
| A-1 | B-2 | C-3 | 50 | 30 | 20 |
| A-2 | B-2 | C-3 | 50 | 30 | 20 |
| A-3 | B-2 | C-3 | 50 | 30 | 20 |
| A-4 | B-2 | C-3 | 50 | 30 | 20 |
| A-5 | B-2 | C-3 | 50 | 30 | 20 |
| A-6 | B-2 | C-3 | 50 | 30 | 20 |
| A-7 | B-2 | C-3 | 50 | 30 | 20 |
| A-8 | B-2 | C-3 | 50 | 30 | 20 |
| A-9 | B-2 | C-3 | 50 | 30 | 20 |
| A-10 | B-2 | C-3 | 50 | 30 | 20 |
| A-11 | B-2 | C-3 | 50 | 30 | 20 |
| A-12 | B-2 | C-3 | 50 | 30 | 20 |
| A-13 | B-2 | C-3 | 50 | 30 | 20 |
| A-14 | B-2 | C-3 | 50 | 30 | 20 |
| A-15 | B-2 | C-3 | 50 | 30 | 20 |
| A-16 | B-2 | C-3 | 50 | 30 | 20 |
| A-17 | B-2 | C-3 | 50 | 30 | 20 |
| A-18 | B-2 | C-3 | 50 | 30 | 20 |
| A-19 | B-2 | C-3 | 50 | 30 | 20 |
| A-20 | B-2 | C-3 | 50 | 30 | 20 |
| A-21 | B-2 | C-3 | 50 | 30 | 20 |
| A-22 | B-2 | C-3 | 50 | 30 | 20 |
| A-23 | B-2 | C-3 | 50 | 30 | 20 |
| A-24 | B-2 | C-3 | 50 | 30 | 20 |
| A-25 | B-2 | C-3 | 50 | 30 | 20 |
| A-26 | B-2 | C-3 | 50 | 30 | 20 |
| A-28 | B-2 | C-3 | 50 | 30 | 20 |
| A-29 | B-2 | C-3 | 50 | 30 | 20 |
| A-30 | B-2 | C-3 | 50 | 30 | 20 |
| A-31 | B-2 | C-3 | 50 | 30 | 20 |
| A-32 | B-2 | C-3 | 50 | 30 | 20 |
| A-33 | B-2 | C-3 | 50 | 30 | 20 |
| A-34 | B-2 | C-3 | 50 | 30 | 20 |
| A-35 | B-2 | C-3 | 50 | 30 | 20 |
| A-36 | B-2 | C-3 | 50 | 30 | 20 |
| A-37 | B-2 | C-3 | 50 | 30 | 20 |
| A-38 | B-2 | C-3 | 50 | 30 | 20 |
| A-39 | B-2 | C-3 | 50 | 30 | 20 |
| A-40 | B-2 | C-3 | 50 | 30 | 20 |
| A-41 | B-2 | C-3 | 50 | 30 | 20 |
| A-42 | B-2 | C-3 | 50 | 30 | 20 |
| A-43 | B-2 | C-3 | 50 | 30 | 20 |
| A-44 | B-2 | C-3 | 50 | 30 | 20 |
| A-45 | B-2 | C-3 | 50 | 30 | 20 |
| A-46 | B-2 | C-3 | 50 | 30 | 20 |
| A-47 | B-2 | C-3 | 50 | 30 | 20 |
| A-49 | B-2 | C-3 | 50 | 30 | 20 |
| A-50 | B-2 | C-3 | 50 | 30 | 20 |
| A-51 | B-2 | C-3 | 50 | 30 | 20 |
| A-52 | B-2 | C-3 | 50 | 30 | 20 |
| A-53 | B-2 | C-3 | 50 | 30 | 20 |
| A-54 | B-2 | C-3 | 50 | 30 | 20 |
| A-55 | B-2 | C-3 | 50 | 30 | 20 |
| A-56 | B-2 | C-3 | 50 | 30 | 20 |
| A-57 | B-2 | C-3 | 50 | 30 | 20 |
| A-58 | B-2 | C-3 | 50 | 30 | 20 |
| A-59 | B-2 | C-3 | 50 | 30 | 20 |
| A-60 | B-2 | C-3 | 50 | 30 | 20 |

TABLE 74

| | | | Ratio of units (mol %) | | |
|---|---|---|---|---|---|
| A | B | C | A | B | C |
| A-61 | B-2 | C-3 | 50 | 30 | 20 |
| A-62 | B-2 | C-3 | 50 | 30 | 20 |
| A-63 | B-2 | C-3 | 50 | 30 | 20 |
| A-64 | B-2 | C-3 | 50 | 30 | 20 |
| A-65 | B-2 | C-3 | 50 | 30 | 20 |
| A-66 | B-2 | C-3 | 50 | 30 | 20 |
| A-67 | B-2 | C-3 | 50 | 30 | 20 |
| A-68 | B-2 | C-3 | 50 | 30 | 20 |
| A-69 | B-2 | C-3 | 50 | 30 | 20 |
| A-70 | B-2 | C-3 | 50 | 30 | 20 |
| A-71 | B-2 | C-3 | 50 | 30 | 20 |
| A-72 | B-2 | C-3 | 50 | 30 | 20 |
| A-73 | B-2 | C-3 | 50 | 30 | 20 |
| A-74 | B-2 | C-3 | 50 | 30 | 20 |
| A-75 | B-2 | C-3 | 50 | 30 | 20 |
| A-76 | B-2 | C-3 | 50 | 30 | 20 |
| A-77 | B-2 | C-3 | 50 | 30 | 20 |
| A-78 | B-2 | C-3 | 50 | 30 | 20 |
| A-79 | B-2 | C-3 | 50 | 30 | 20 |
| A-80 | B-2 | C-3 | 50 | 30 | 20 |
| A-81 | B-2 | C-3 | 50 | 30 | 20 |
| A-82 | B-2 | C-3 | 50 | 30 | 20 |
| A-83 | B-2 | C-3 | 50 | 30 | 20 |
| A-84 | B-2 | C-3 | 50 | 30 | 20 |
| A-85 | B-2 | C-3 | 50 | 30 | 20 |
| A-86 | B-2 | C-3 | 50 | 30 | 20 |
| A-87 | B-2 | C-3 | 50 | 30 | 20 |
| A-88 | B-2 | C-3 | 50 | 30 | 20 |
| A-89 | B-2 | C-3 | 50 | 30 | 20 |
| A-90 | B-2 | C-3 | 50 | 30 | 20 |
| A-91 | B-2 | C-3 | 50 | 30 | 20 |
| A-92 | B-2 | C-3 | 50 | 30 | 20 |
| A-93 | B-2 | C-3 | 50 | 30 | 20 |
| A-94 | B-2 | C-3 | 50 | 30 | 20 |
| A-95 | B-2 | C-3 | 50 | 30 | 20 |
| A-96 | B-2 | C-3 | 50 | 30 | 20 |
| A-100 | B-2 | C-3 | 50 | 30 | 20 |
| A-101 | B-2 | C-3 | 50 | 30 | 20 |
| A-102 | B-2 | C-3 | 50 | 30 | 20 |
| A-103 | B-2 | C-3 | 50 | 30 | 20 |
| A-104 | B-2 | C-3 | 50 | 30 | 20 |
| A-105 | B-2 | C-3 | 50 | 30 | 20 |
| A-106 | B-2 | C-3 | 50 | 30 | 20 |
| A-107 | B-2 | C-3 | 50 | 30 | 20 |
| A-108 | B-2 | C-3 | 50 | 30 | 20 |
| A-109 | B-2 | C-3 | 50 | 30 | 20 |
| A-110 | B-2 | C-3 | 50 | 30 | 20 |
| A-111 | B-2 | C-3 | 50 | 30 | 20 |
| A-112 | B-2 | C-3 | 50 | 30 | 20 |
| A-113 | B-2 | C-3 | 50 | 30 | 20 |
| A-114 | B-2 | C-3 | 50 | 30 | 20 |
| A-115 | B-2 | C-3 | 50 | 30 | 20 |
| A-116 | B-2 | C-3 | 50 | 30 | 20 |
| A-117 | B-2 | C-3 | 50 | 30 | 20 |
| A-118 | B-2 | C-3 | 50 | 30 | 20 |
| A-119 | B-2 | C-3 | 50 | 30 | 20 |
| A-120 | B-2 | C-3 | 50 | 30 | 20 |
| A-121 | B-2 | C-3 | 50 | 30 | 20 |
| A-122 | B-2 | C-3 | 50 | 30 | 20 |
| A-123 | B-2 | C-3 | 50 | 30 | 20 |

TABLE 75

| | | | Ratio of units (mol %) | | |
|---|---|---|---|---|---|
| A | B | C | A | B | C |
| A-124 | B-2 | C-3 | 50 | 30 | 20 |
| A-125 | B-2 | C-3 | 50 | 30 | 20 |
| A-126 | B-2 | C-3 | 50 | 30 | 20 |
| A-127 | B-2 | C-3 | 50 | 30 | 20 |
| A-128 | B-2 | C-3 | 50 | 30 | 20 |
| A-129 | B-2 | C-3 | 50 | 30 | 20 |
| A-130 | B-2 | C-3 | 50 | 30 | 20 |
| A-131 | B-2 | C-3 | 50 | 30 | 20 |
| A-132 | B-2 | C-3 | 50 | 30 | 20 |
| A-133 | B-2 | C-3 | 50 | 30 | 20 |
| A-134 | B-2 | C-3 | 50 | 30 | 20 |
| A-135 | B-2 | C-3 | 50 | 30 | 20 |
| A-136 | B-2 | C-3 | 50 | 30 | 20 |
| A-137 | B-2 | C-3 | 50 | 30 | 20 |
| A-138 | B-2 | C-3 | 50 | 30 | 20 |
| A-139 | B-2 | C-3 | 50 | 30 | 20 |
| A-140 | B-2 | C-3 | 50 | 30 | 20 |
| A-141 | B-2 | C-3 | 50 | 30 | 20 |
| A-142 | B-2 | C-3 | 50 | 30 | 20 |
| A-143 | B-2 | C-3 | 50 | 30 | 20 |
| A-144 | B-2 | C-3 | 50 | 30 | 20 |
| A-145 | B-2 | C-3 | 50 | 30 | 20 |
| A-146 | B-2 | C-3 | 50 | 30 | 20 |
| A-147 | B-2 | C-3 | 50 | 30 | 20 |
| A-160 | B-2 | C-3 | 50 | 30 | 20 |
| A-161 | B-2 | C-3 | 50 | 30 | 20 |
| A-162 | B-2 | C-3 | 50 | 30 | 20 |
| A-163 | B-2 | C-3 | 50 | 30 | 20 |
| A-164 | B-2 | C-3 | 50 | 30 | 20 |
| A-165 | B-2 | C-3 | 50 | 30 | 20 |
| A-166 | B-2 | C-3 | 50 | 30 | 20 |
| A-167 | B-2 | C-3 | 50 | 30 | 20 |
| A-168 | B-2 | C-3 | 50 | 30 | 20 |
| A-169 | B-2 | C-3 | 50 | 30 | 20 |
| A-170 | B-2 | C-3 | 50 | 30 | 20 |
| A-171 | B-2 | C-3 | 50 | 30 | 20 |
| A-172 | B-2 | C-3 | 50 | 30 | 20 |
| A-173 | B-2 | C-3 | 50 | 30 | 20 |
| A-174 | B-2 | C-3 | 50 | 30 | 20 |
| A-175 | B-2 | C-3 | 50 | 30 | 20 |
| A-176 | B-2 | C-3 | 50 | 30 | 20 |
| A-177 | B-2 | C-3 | 50 | 30 | 20 |
| A-178 | B-2 | C-3 | 50 | 30 | 20 |
| A-179 | B-2 | C-3 | 50 | 30 | 20 |
| A-180 | B-2 | C-3 | 50 | 30 | 20 |
| A-181 | B-2 | C-3 | 50 | 30 | 20 |
| A-182 | B-2 | C-3 | 50 | 30 | 20 |
| A-183 | B-2 | C-3 | 50 | 30 | 20 |
| A-190 | B-2 | C-3 | 50 | 30 | 20 |
| A-191 | B-2 | C-3 | 50 | 30 | 20 |
| A-192 | B-2 | C-3 | 50 | 30 | 20 |
| A-193 | B-2 | C-3 | 50 | 30 | 20 |
| A-194 | B-2 | C-3 | 50 | 30 | 20 |
| A-195 | B-2 | C-3 | 50 | 30 | 20 |
| A-196 | B-2 | C-3 | 50 | 30 | 20 |
| A-197 | B-2 | C-3 | 50 | 30 | 20 |
| A-198 | B-2 | C-3 | 50 | 30 | 20 |
| A-199 | B-2 | C-3 | 50 | 30 | 20 |
| A-200 | B-2 | C-3 | 50 | 30 | 20 |
| A-201 | B-2 | C-3 | 50 | 30 | 20 |
| A-202 | B-2 | C-3 | 50 | 30 | 20 |
| A-203 | B-2 | C-3 | 50 | 30 | 20 |
| A-204 | B-2 | C-3 | 50 | 30 | 20 |
| A-205 | B-2 | C-3 | 50 | 30 | 20 |
| A-206 | B-2 | C-3 | 50 | 30 | 20 |
| A-207 | B-2 | C-3 | 50 | 30 | 20 |
| A-208 | B-2 | C-3 | 50 | 30 | 20 |
| A-209 | B-2 | C-3 | 50 | 30 | 20 |
| A-210 | B-2 | C-3 | 50 | 30 | 20 |
| A-211 | B-2 | C-3 | 50 | 30 | 20 |
| A-212 | B-2 | C-3 | 50 | 30 | 20 |
| A-213 | B-2 | C-3 | 50 | 30 | 20 |

TABLE 76

| | | | Ratio of units (mol %) | | |
|---|---|---|---|---|---|
| A | B | C | A | B | C |
| A-220 | B-2 | C-3 | 50 | 30 | 20 |
| A-221 | B-2 | C-3 | 50 | 30 | 20 |
| A-222 | B-2 | C-3 | 50 | 30 | 20 |
| A-223 | B-2 | C-3 | 50 | 30 | 20 |
| A-224 | B-2 | C-3 | 50 | 30 | 20 |
| A-225 | B-2 | C-3 | 50 | 30 | 20 |
| A-226 | B-2 | C-3 | 50 | 30 | 20 |
| A-227 | B-2 | C-3 | 50 | 30 | 20 |
| A-228 | B-2 | C-3 | 50 | 30 | 20 |
| A-229 | B-2 | C-3 | 50 | 30 | 20 |
| A-230 | B-2 | C-3 | 50 | 30 | 20 |
| A-231 | B-2 | C-3 | 50 | 30 | 20 |
| A-232 | B-2 | C-3 | 50 | 30 | 20 |
| A-233 | B-2 | C-3 | 50 | 30 | 20 |
| A-234 | B-2 | C-3 | 50 | 30 | 20 |
| A-235 | B-2 | C-3 | 50 | 30 | 20 |
| A-236 | B-2 | C-3 | 50 | 30 | 20 |
| A-237 | B-2 | C-3 | 50 | 30 | 20 |
| A-238 | B-2 | C-3 | 50 | 30 | 20 |
| A-239 | B-2 | C-3 | 50 | 30 | 20 |
| A-240 | B-2 | C-3 | 50 | 30 | 20 |

TABLE 77

| | | | Ratio of units (mol %) | | |
|---|---|---|---|---|---|
| A | B | C | A | B | C |
| A-1 | B-2 | C-4 | 50 | 30 | 20 |
| A-2 | B-2 | C-4 | 50 | 30 | 20 |
| A-3 | B-2 | C-4 | 50 | 30 | 20 |
| A-4 | B-2 | C-4 | 50 | 30 | 20 |
| A-5 | B-2 | C-4 | 50 | 30 | 20 |
| A-6 | B-2 | C-4 | 50 | 30 | 20 |
| A-7 | B-2 | C-4 | 50 | 30 | 20 |
| A-8 | B-2 | C-4 | 50 | 30 | 20 |
| A-9 | B-2 | C-4 | 50 | 30 | 20 |
| A-10 | B-2 | C-4 | 50 | 30 | 20 |
| A-11 | B-2 | C-4 | 50 | 30 | 20 |
| A-12 | B-2 | C-4 | 50 | 30 | 20 |
| A-13 | B-2 | C-4 | 50 | 30 | 20 |
| A-14 | B-2 | C-4 | 50 | 30 | 20 |
| A-15 | B-2 | C-4 | 50 | 30 | 20 |
| A-16 | B-2 | C-4 | 50 | 30 | 20 |
| A-17 | B-2 | C-4 | 50 | 30 | 20 |
| A-18 | B-2 | C-4 | 50 | 30 | 20 |
| A-19 | B-2 | C-4 | 50 | 30 | 20 |
| A-20 | B-2 | C-4 | 50 | 30 | 20 |
| A-21 | B-2 | C-4 | 50 | 30 | 20 |
| A-22 | B-2 | C-4 | 50 | 30 | 20 |
| A-23 | B-2 | C-4 | 50 | 30 | 20 |
| A-24 | B-2 | C-4 | 50 | 30 | 20 |
| A-25 | B-2 | C-4 | 50 | 30 | 20 |
| A-26 | B-2 | C-4 | 50 | 30 | 20 |
| A-28 | B-2 | C-4 | 50 | 30 | 20 |
| A-29 | B-2 | C-4 | 50 | 30 | 20 |
| A-30 | B-2 | C-4 | 50 | 30 | 20 |
| A-31 | B-2 | C-4 | 50 | 30 | 20 |
| A-32 | B-2 | C-4 | 50 | 30 | 20 |
| A-33 | B-2 | C-4 | 50 | 30 | 20 |
| A-34 | B-2 | C-4 | 50 | 30 | 20 |
| A-35 | B-2 | C-4 | 50 | 30 | 20 |
| A-36 | B-2 | C-4 | 50 | 30 | 20 |
| A-37 | B-2 | C-4 | 50 | 30 | 20 |
| A-38 | B-2 | C-4 | 50 | 30 | 20 |
| A-39 | B-2 | C-4 | 50 | 30 | 20 |
| A-40 | B-2 | C-4 | 50 | 30 | 20 |
| A-41 | B-2 | C-4 | 50 | 30 | 20 |
| A-42 | B-2 | C-4 | 50 | 30 | 20 |
| A-43 | B-2 | C-4 | 50 | 30 | 20 |
| A-44 | B-2 | C-4 | 50 | 30 | 20 |
| A-45 | B-2 | C-4 | 50 | 30 | 20 |
| A-46 | B-2 | C-4 | 50 | 30 | 20 |
| A-47 | B-2 | C-4 | 50 | 30 | 20 |
| A-49 | B-2 | C-4 | 50 | 30 | 20 |
| A-50 | B-2 | C-4 | 50 | 30 | 20 |
| A-51 | B-2 | C-4 | 50 | 30 | 20 |
| A-52 | B-2 | C-4 | 50 | 30 | 20 |
| A-53 | B-2 | C-4 | 50 | 30 | 20 |
| A-54 | B-2 | C-4 | 50 | 30 | 20 |
| A-55 | B-2 | C-4 | 50 | 30 | 20 |
| A-56 | B-2 | C-4 | 50 | 30 | 20 |
| A-57 | B-2 | C-4 | 50 | 30 | 20 |
| A-58 | B-2 | C-4 | 50 | 30 | 20 |
| A-59 | B-2 | C-4 | 50 | 30 | 20 |
| A-60 | B-2 | C-4 | 50 | 30 | 20 |

TABLE 78

| | | | Ratio of units (mol %) | | |
|---|---|---|---|---|---|
| A | B | C | A | B | C |
| A-61 | B-2 | C-4 | 50 | 30 | 20 |
| A-62 | B-2 | C-4 | 50 | 30 | 20 |
| A-63 | B-2 | C-4 | 50 | 30 | 20 |
| A-64 | B-2 | C-4 | 50 | 30 | 20 |
| A-65 | B-2 | C-4 | 50 | 30 | 20 |
| A-66 | B-2 | C-4 | 50 | 30 | 20 |
| A-67 | B-2 | C-4 | 50 | 30 | 20 |
| A-68 | B-2 | C-4 | 50 | 30 | 20 |
| A-69 | B-2 | C-4 | 50 | 30 | 20 |
| A-70 | B-2 | C-4 | 50 | 30 | 20 |
| A-71 | B-2 | C-4 | 50 | 30 | 20 |
| A-72 | B-2 | C-4 | 50 | 30 | 20 |
| A-73 | B-2 | C-4 | 50 | 30 | 20 |
| A-74 | B-2 | C-4 | 50 | 30 | 20 |
| A-75 | B-2 | C-4 | 50 | 30 | 20 |
| A-76 | B-2 | C-4 | 50 | 30 | 20 |
| A-77 | B-2 | C-4 | 50 | 30 | 20 |
| A-78 | B-2 | C-4 | 50 | 30 | 20 |
| A-79 | B-2 | C-4 | 50 | 30 | 20 |
| A-80 | B-2 | C-4 | 50 | 30 | 20 |
| A-81 | B-2 | C-4 | 50 | 30 | 20 |
| A-82 | B-2 | C-4 | 50 | 30 | 20 |
| A-83 | B-2 | C-4 | 50 | 30 | 20 |
| A-84 | B-2 | C-4 | 50 | 30 | 20 |
| A-85 | B-2 | C-4 | 50 | 30 | 20 |
| A-86 | B-2 | C-4 | 50 | 30 | 20 |
| A-87 | B-2 | C-4 | 50 | 30 | 20 |
| A-88 | B-2 | C-4 | 50 | 30 | 20 |
| A-89 | B-2 | C-4 | 50 | 30 | 20 |
| A-90 | B-2 | C-4 | 50 | 30 | 20 |
| A-91 | B-2 | C-4 | 50 | 30 | 20 |
| A-92 | B-2 | C-4 | 50 | 30 | 20 |
| A-93 | B-2 | C-4 | 50 | 30 | 20 |
| A-94 | B-2 | C-4 | 50 | 30 | 20 |
| A-95 | B-2 | C-4 | 50 | 30 | 20 |
| A-96 | B-2 | C-4 | 50 | 30 | 20 |
| A-100 | B-2 | C-4 | 50 | 30 | 20 |
| A-101 | B-2 | C-4 | 50 | 30 | 20 |
| A-102 | B-2 | C-4 | 50 | 30 | 20 |
| A-103 | B-2 | C-4 | 50 | 30 | 20 |
| A-104 | B-2 | C-4 | 50 | 30 | 20 |
| A-105 | B-2 | C-4 | 50 | 30 | 20 |
| A-106 | B-2 | C-4 | 50 | 30 | 20 |
| A-107 | B-2 | C-4 | 50 | 30 | 20 |
| A-108 | B-2 | C-4 | 50 | 30 | 20 |
| A-109 | B-2 | C-4 | 50 | 30 | 20 |
| A-110 | B-2 | C-4 | 50 | 30 | 20 |
| A-111 | B-2 | C-4 | 50 | 30 | 20 |
| A-112 | B-2 | C-4 | 50 | 30 | 20 |
| A-113 | B-2 | C-4 | 50 | 30 | 20 |
| A-114 | B-2 | C-4 | 50 | 30 | 20 |
| A-115 | B-2 | C-4 | 50 | 30 | 20 |
| A-116 | B-2 | C-4 | 50 | 30 | 20 |
| A-117 | B-2 | C-4 | 50 | 30 | 20 |
| A-118 | B-2 | C-4 | 50 | 30 | 20 |
| A-119 | B-2 | C-4 | 50 | 30 | 20 |
| A-120 | B-2 | C-4 | 50 | 30 | 20 |
| A-121 | B-2 | C-4 | 50 | 30 | 20 |
| A-122 | B-2 | C-4 | 50 | 30 | 20 |
| A-123 | B-2 | C-4 | 50 | 30 | 20 |

TABLE 79

| | | | Ratio of units (mol %) | | |
|---|---|---|---|---|---|
| A | B | C | A | B | C |
| A-124 | B-2 | C-4 | 50 | 30 | 20 |
| A-125 | B-2 | C-4 | 50 | 30 | 20 |
| A-126 | B-2 | C-4 | 50 | 30 | 20 |
| A-127 | B-2 | C-4 | 50 | 30 | 20 |
| A-128 | B-2 | C-4 | 50 | 30 | 20 |
| A-129 | B-2 | C-4 | 50 | 30 | 20 |
| A-130 | B-2 | C-4 | 50 | 30 | 20 |
| A-131 | B-2 | C-4 | 50 | 30 | 20 |
| A-132 | B-2 | C-4 | 50 | 30 | 20 |
| A-133 | B-2 | C-4 | 50 | 30 | 20 |
| A-134 | B-2 | C-4 | 50 | 30 | 20 |
| A-135 | B-2 | C-4 | 50 | 30 | 20 |
| A-136 | B-2 | C-4 | 50 | 30 | 20 |
| A-137 | B-2 | C-4 | 50 | 30 | 20 |
| A-138 | B-2 | C-4 | 50 | 30 | 20 |
| A-139 | B-2 | C-4 | 50 | 30 | 20 |
| A-140 | B-2 | C-4 | 50 | 30 | 20 |
| A-141 | B-2 | C-4 | 50 | 30 | 20 |
| A-142 | B-2 | C-4 | 50 | 30 | 20 |
| A-143 | B-2 | C-4 | 50 | 30 | 20 |
| A-144 | B-2 | C-4 | 50 | 30 | 20 |
| A-145 | B-2 | C-4 | 50 | 30 | 20 |
| A-146 | B-2 | C-4 | 50 | 30 | 20 |
| A-147 | B-2 | C-4 | 50 | 30 | 20 |
| A-160 | B-2 | C-4 | 50 | 30 | 20 |
| A-161 | B-2 | C-4 | 50 | 30 | 20 |
| A-162 | B-2 | C-4 | 50 | 30 | 20 |
| A-163 | B-2 | C-4 | 50 | 30 | 20 |
| A-164 | B-2 | C-4 | 50 | 30 | 20 |
| A-165 | B-2 | C-4 | 50 | 30 | 20 |
| A-166 | B-2 | C-4 | 50 | 30 | 20 |
| A-167 | B-2 | C-4 | 50 | 30 | 20 |
| A-168 | B-2 | C-4 | 50 | 30 | 20 |
| A-169 | B-2 | C-4 | 50 | 30 | 20 |
| A-170 | B-2 | C-4 | 50 | 30 | 20 |
| A-171 | B-2 | C-4 | 50 | 30 | 20 |
| A-172 | B-2 | C-4 | 50 | 30 | 20 |
| A-173 | B-2 | C-4 | 50 | 30 | 20 |
| A-174 | B-2 | C-4 | 50 | 30 | 20 |
| A-175 | B-2 | C-4 | 50 | 30 | 20 |
| A-176 | B-2 | C-4 | 50 | 30 | 20 |
| A-177 | B-2 | C-4 | 50 | 30 | 20 |
| A-178 | B-2 | C-4 | 50 | 30 | 20 |
| A-179 | B-2 | C-4 | 50 | 30 | 20 |
| A-180 | B-2 | C-4 | 50 | 30 | 20 |
| A-181 | B-2 | C-4 | 50 | 30 | 20 |
| A-182 | B-2 | C-4 | 50 | 30 | 20 |
| A-183 | B-2 | C-4 | 50 | 30 | 20 |
| A-190 | B-2 | C-4 | 50 | 30 | 20 |
| A-191 | B-2 | C-4 | 50 | 30 | 20 |
| A-192 | B-2 | C-4 | 50 | 30 | 20 |
| A-193 | B-2 | C-4 | 50 | 30 | 20 |
| A-194 | B-2 | C-4 | 50 | 30 | 20 |
| A-195 | B-2 | C-4 | 50 | 30 | 20 |
| A-196 | B-2 | C-4 | 50 | 30 | 20 |
| A-197 | B-2 | C-4 | 50 | 30 | 20 |
| A-198 | B-2 | C-4 | 50 | 30 | 20 |
| A-199 | B-2 | C-4 | 50 | 30 | 20 |
| A-200 | B-2 | C-4 | 50 | 30 | 20 |
| A-201 | B-2 | C-4 | 50 | 30 | 20 |
| A-202 | B-2 | C-4 | 50 | 30 | 20 |
| A-203 | B-2 | C-4 | 50 | 30 | 20 |
| A-204 | B-2 | C-4 | 50 | 30 | 20 |
| A-205 | B-2 | C-4 | 50 | 30 | 20 |
| A-206 | B-2 | C-4 | 50 | 30 | 20 |
| A-207 | B-2 | C-4 | 50 | 30 | 20 |
| A-208 | B-2 | C-4 | 50 | 30 | 20 |
| A-209 | B-2 | C-4 | 50 | 30 | 20 |
| A-210 | B-2 | C-4 | 50 | 30 | 20 |
| A-211 | B-2 | C-4 | 50 | 30 | 20 |
| A-212 | B-2 | C-4 | 50 | 30 | 20 |
| A-213 | B-2 | C-4 | 50 | 30 | 20 |

TABLE 80

| | | | Ratio of units (mol %) | | |
|---|---|---|---|---|---|
| A | B | C | A | B | C |
| A-220 | B-2 | C-4 | 50 | 30 | 20 |
| A-221 | B-2 | C-4 | 50 | 30 | 20 |
| A-222 | B-2 | C-4 | 50 | 30 | 20 |
| A-223 | B-2 | C-4 | 50 | 30 | 20 |
| A-224 | B-2 | C-4 | 50 | 30 | 20 |
| A-225 | B-2 | C-4 | 50 | 30 | 20 |
| A-226 | B-2 | C-4 | 50 | 30 | 20 |
| A-227 | B-2 | C-4 | 50 | 30 | 20 |
| A-228 | B-2 | C-4 | 50 | 30 | 20 |
| A-229 | B-2 | C-4 | 50 | 30 | 20 |
| A-230 | B-2 | C-4 | 50 | 30 | 20 |
| A-231 | B-2 | C-4 | 50 | 30 | 20 |
| A-232 | B-2 | C-4 | 50 | 30 | 20 |
| A-233 | B-2 | C-4 | 50 | 30 | 20 |
| A-234 | B-2 | C-4 | 50 | 30 | 20 |
| A-235 | B-2 | C-4 | 50 | 30 | 20 |
| A-236 | B-2 | C-4 | 50 | 30 | 20 |
| A-237 | B-2 | C-4 | 50 | 30 | 20 |
| A-238 | B-2 | C-4 | 50 | 30 | 20 |
| A-239 | B-2 | C-4 | 50 | 30 | 20 |
| A-240 | B-2 | C-4 | 50 | 30 | 20 |

TABLE 81

| | | | Ratio of units (mol %) | | |
|---|---|---|---|---|---|
| A | B | C | A | B | C |
| A-1 | B-3 | C-1 | 50 | 45 | 5 |
| A-2 | B-3 | C-1 | 50 | 45 | 5 |
| A-3 | B-3 | C-1 | 50 | 45 | 5 |
| A-4 | B-3 | C-1 | 50 | 45 | 5 |
| A-5 | B-3 | C-1 | 50 | 45 | 5 |
| A-6 | B-3 | C-1 | 50 | 45 | 5 |
| A-7 | B-3 | C-1 | 50 | 45 | 5 |
| A-8 | B-3 | C-1 | 50 | 45 | 5 |
| A-9 | B-3 | C-1 | 50 | 45 | 5 |
| A-10 | B-3 | C-1 | 50 | 45 | 5 |
| A-11 | B-3 | C-1 | 50 | 45 | 5 |
| A-12 | B-3 | C-1 | 50 | 45 | 5 |
| A-13 | B-3 | C-1 | 50 | 45 | 5 |
| A-14 | B-3 | C-1 | 50 | 45 | 5 |
| A-15 | B-3 | C-1 | 50 | 45 | 5 |
| A-16 | B-3 | C-1 | 50 | 45 | 5 |
| A-17 | B-3 | C-1 | 50 | 45 | 5 |
| A-18 | B-3 | C-1 | 50 | 45 | 5 |
| A-19 | B-3 | C-1 | 50 | 45 | 5 |
| A-20 | B-3 | C-1 | 50 | 45 | 5 |
| A-21 | B-3 | C-1 | 50 | 45 | 5 |
| A-22 | B-3 | C-1 | 50 | 45 | 5 |
| A-23 | B-3 | C-1 | 50 | 45 | 5 |
| A-24 | B-3 | C-1 | 50 | 45 | 5 |
| A-25 | B-3 | C-1 | 50 | 45 | 5 |
| A-26 | B-3 | C-1 | 50 | 45 | 5 |
| A-28 | B-3 | C-1 | 50 | 45 | 5 |
| A-29 | B-3 | C-1 | 50 | 45 | 5 |
| A-30 | B-3 | C-1 | 50 | 45 | 5 |
| A-31 | B-3 | C-1 | 50 | 45 | 5 |
| A-32 | B-3 | C-1 | 50 | 45 | 5 |
| A-33 | B-3 | C-1 | 50 | 45 | 5 |
| A-34 | B-3 | C-1 | 50 | 45 | 5 |
| A-35 | B-3 | C-1 | 50 | 45 | 5 |
| A-36 | B-3 | C-1 | 50 | 45 | 5 |
| A-37 | B-3 | C-1 | 50 | 45 | 5 |
| A-38 | B-3 | C-1 | 50 | 45 | 5 |
| A-39 | B-3 | C-1 | 50 | 45 | 5 |
| A-40 | B-3 | C-1 | 50 | 45 | 5 |
| A-41 | B-3 | C-1 | 50 | 45 | 5 |
| A-42 | B-3 | C-1 | 50 | 45 | 5 |
| A-43 | B-3 | C-1 | 50 | 45 | 5 |
| A-44 | B-3 | C-1 | 50 | 45 | 5 |
| A-45 | B-3 | C-1 | 50 | 45 | 5 |
| A-46 | B-3 | C-1 | 50 | 45 | 5 |
| A-47 | B-3 | C-1 | 50 | 45 | 5 |
| A-49 | B-3 | C-1 | 50 | 45 | 5 |
| A-50 | B-3 | C-1 | 50 | 45 | 5 |
| A-51 | B-3 | C-1 | 50 | 45 | 5 |
| A-52 | B-3 | C-1 | 50 | 45 | 5 |
| A-53 | B-3 | C-1 | 50 | 45 | 5 |
| A-54 | B-3 | C-1 | 50 | 45 | 5 |
| A-55 | B-3 | C-1 | 50 | 45 | 5 |
| A-56 | B-3 | C-1 | 50 | 45 | 5 |
| A-57 | B-3 | C-1 | 50 | 45 | 5 |
| A-58 | B-3 | C-1 | 50 | 45 | 5 |
| A-59 | B-3 | C-1 | 50 | 45 | 5 |
| A-60 | B-3 | C-1 | 50 | 45 | 5 |

TABLE 82

| | | | Ratio of units (mol %) | | |
|---|---|---|---|---|---|
| A | B | C | A | B | C |
| A-61 | B-3 | C-1 | 50 | 45 | 5 |
| A-62 | B-3 | C-1 | 50 | 45 | 5 |
| A-63 | B-3 | C-1 | 50 | 45 | 5 |
| A-64 | B-3 | C-1 | 50 | 45 | 5 |
| A-65 | B-3 | C-1 | 50 | 45 | 5 |
| A-66 | B-3 | C-1 | 50 | 45 | 5 |
| A-67 | B-3 | C-1 | 50 | 45 | 5 |
| A-68 | B-3 | C-1 | 50 | 45 | 5 |
| A-69 | B-3 | C-1 | 50 | 45 | 5 |
| A-70 | B-3 | C-1 | 50 | 45 | 5 |
| A-71 | B-3 | C-1 | 50 | 45 | 5 |
| A-72 | B-3 | C-1 | 50 | 45 | 5 |
| A-73 | B-3 | C-1 | 50 | 45 | 5 |
| A-74 | B-3 | C-1 | 50 | 45 | 5 |
| A-75 | B-3 | C-1 | 50 | 45 | 5 |
| A-76 | B-3 | C-1 | 50 | 45 | 5 |
| A-77 | B-3 | C-1 | 50 | 45 | 5 |
| A-78 | B-3 | C-1 | 50 | 45 | 5 |
| A-79 | B-3 | C-1 | 50 | 45 | 5 |
| A-80 | B-3 | C-1 | 50 | 45 | 5 |
| A-81 | B-3 | C-1 | 50 | 45 | 5 |
| A-82 | B-3 | C-1 | 50 | 45 | 5 |
| A-83 | B-3 | C-1 | 50 | 45 | 5 |
| A-84 | B-3 | C-1 | 50 | 45 | 5 |
| A-85 | B-3 | C-1 | 50 | 45 | 5 |
| A-86 | B-3 | C-1 | 50 | 45 | 5 |
| A-87 | B-3 | C-1 | 50 | 45 | 5 |
| A-88 | B-3 | C-1 | 50 | 45 | 5 |
| A-89 | B-3 | C-1 | 50 | 45 | 5 |
| A-90 | B-3 | C-1 | 50 | 45 | 5 |
| A-91 | B-3 | C-1 | 50 | 45 | 5 |
| A-92 | B-3 | C-1 | 50 | 45 | 5 |
| A-93 | B-3 | C-1 | 50 | 45 | 5 |
| A-94 | B-3 | C-1 | 50 | 45 | 5 |
| A-95 | B-3 | C-1 | 50 | 45 | 5 |
| A-96 | B-3 | C-1 | 50 | 45 | 5 |
| A-100 | B-3 | C-1 | 50 | 45 | 5 |
| A-101 | B-3 | C-1 | 50 | 45 | 5 |
| A-102 | B-3 | C-1 | 50 | 45 | 5 |
| A-103 | B-3 | C-1 | 50 | 45 | 5 |
| A-104 | B-3 | C-1 | 50 | 45 | 5 |
| A-105 | B-3 | C-1 | 50 | 45 | 5 |
| A-106 | B-3 | C-1 | 50 | 45 | 5 |
| A-107 | B-3 | C-1 | 50 | 45 | 5 |
| A-108 | B-3 | C-1 | 50 | 45 | 5 |
| A-109 | B-3 | C-1 | 50 | 45 | 5 |
| A-110 | B-3 | C-1 | 50 | 45 | 5 |
| A-111 | B-3 | C-1 | 50 | 45 | 5 |
| A-112 | B-3 | C-1 | 50 | 45 | 5 |
| A-113 | B-3 | C-1 | 50 | 45 | 5 |
| A-114 | B-3 | C-1 | 50 | 45 | 5 |
| A-115 | B-3 | C-1 | 50 | 45 | 5 |
| A-116 | B-3 | C-1 | 50 | 45 | 5 |
| A-117 | B-3 | C-1 | 50 | 45 | 5 |
| A-118 | B-3 | C-1 | 50 | 45 | 5 |
| A-119 | B-3 | C-1 | 50 | 45 | 5 |
| A-120 | B-3 | C-1 | 50 | 45 | 5 |
| A-121 | B-3 | C-1 | 50 | 45 | 5 |
| A-122 | B-3 | C-1 | 50 | 45 | 5 |
| A-123 | B-3 | C-1 | 50 | 45 | 5 |

TABLE 83

| | | | Ratio of units (mol %) | | |
|---|---|---|---|---|---|
| A | B | C | A | B | C |
| A-124 | B-3 | C-1 | 50 | 45 | 5 |
| A-125 | B-3 | C-1 | 50 | 45 | 5 |
| A-126 | B-3 | C-1 | 50 | 45 | 5 |
| A-127 | B-3 | C-1 | 50 | 45 | 5 |
| A-128 | B-3 | C-1 | 50 | 45 | 5 |
| A-129 | B-3 | C-1 | 50 | 45 | 5 |
| A-130 | B-3 | C-1 | 50 | 45 | 5 |
| A-131 | B-3 | C-1 | 50 | 45 | 5 |
| A-132 | B-3 | C-1 | 50 | 45 | 5 |
| A-133 | B-3 | C-1 | 50 | 45 | 5 |
| A-134 | B-3 | C-1 | 50 | 45 | 5 |
| A-135 | B-3 | C-1 | 50 | 45 | 5 |
| A-136 | B-3 | C-1 | 50 | 45 | 5 |
| A-137 | B-3 | C-1 | 50 | 45 | 5 |
| A-138 | B-3 | C-1 | 50 | 45 | 5 |
| A-139 | B-3 | C-1 | 50 | 45 | 5 |
| A-140 | B-3 | C-1 | 50 | 45 | 5 |
| A-141 | B-3 | C-1 | 50 | 45 | 5 |
| A-142 | B-3 | C-1 | 50 | 45 | 5 |
| A-143 | B-3 | C-1 | 50 | 45 | 5 |
| A-144 | B-3 | C-1 | 50 | 45 | 5 |
| A-145 | B-3 | C-1 | 50 | 45 | 5 |
| A-146 | B-3 | C-1 | 50 | 45 | 5 |
| A-147 | B-3 | C-1 | 50 | 45 | 5 |
| A-160 | B-3 | C-1 | 50 | 45 | 5 |
| A-161 | B-3 | C-1 | 50 | 45 | 5 |
| A-162 | B-3 | C-1 | 50 | 45 | 5 |
| A-163 | B-3 | C-1 | 50 | 45 | 5 |
| A-164 | B-3 | C-1 | 50 | 45 | 5 |
| A-165 | B-3 | C-1 | 50 | 45 | 5 |
| A-166 | B-3 | C-1 | 50 | 45 | 5 |
| A-167 | B-3 | C-1 | 50 | 45 | 5 |
| A-168 | B-3 | C-1 | 50 | 45 | 5 |
| A-169 | B-3 | C-1 | 50 | 45 | 5 |
| A-170 | B-3 | C-1 | 50 | 45 | 5 |
| A-171 | B-3 | C-1 | 50 | 45 | 5 |
| A-172 | B-3 | C-1 | 50 | 45 | 5 |
| A-173 | B-3 | C-1 | 50 | 45 | 5 |
| A-174 | B-3 | C-1 | 50 | 45 | 5 |
| A-175 | B-3 | C-1 | 50 | 45 | 5 |
| A-176 | B-3 | C-1 | 50 | 45 | 5 |
| A-177 | B-3 | C-1 | 50 | 45 | 5 |
| A-178 | B-3 | C-1 | 50 | 45 | 5 |
| A-179 | B-3 | C-1 | 50 | 45 | 5 |
| A-180 | B-3 | C-1 | 50 | 45 | 5 |
| A-181 | B-3 | C-1 | 50 | 45 | 5 |
| A-182 | B-3 | C-1 | 50 | 45 | 5 |
| A-183 | B-3 | C-1 | 50 | 45 | 5 |
| A-190 | B-3 | C-1 | 50 | 45 | 5 |
| A-191 | B-3 | C-1 | 50 | 45 | 5 |
| A-192 | B-3 | C-1 | 50 | 45 | 5 |
| A-193 | B-3 | C-1 | 50 | 45 | 5 |
| A-194 | B-3 | C-1 | 50 | 45 | 5 |
| A-195 | B-3 | C-1 | 50 | 45 | 5 |
| A-196 | B-3 | C-1 | 50 | 45 | 5 |
| A-197 | B-3 | C-1 | 50 | 45 | 5 |
| A-198 | B-3 | C-1 | 50 | 45 | 5 |
| A-199 | B-3 | C-1 | 50 | 45 | 5 |
| A-200 | B-3 | C-1 | 50 | 45 | 5 |
| A-201 | B-3 | C-1 | 50 | 45 | 5 |
| A-202 | B-3 | C-1 | 50 | 45 | 5 |
| A-203 | B-3 | C-1 | 50 | 45 | 5 |
| A-204 | B-3 | C-1 | 50 | 45 | 5 |
| A-205 | B-3 | C-1 | 50 | 45 | 5 |
| A-206 | B-3 | C-1 | 50 | 45 | 5 |
| A-207 | B-3 | C-1 | 50 | 45 | 5 |
| A-208 | B-3 | C-1 | 50 | 45 | 5 |
| A-209 | B-3 | C-1 | 50 | 45 | 5 |
| A-210 | B-3 | C-1 | 50 | 45 | 5 |
| A-211 | B-3 | C-1 | 50 | 45 | 5 |
| A-212 | B-3 | C-1 | 50 | 45 | 5 |
| A-213 | B-3 | C-1 | 50 | 45 | 5 |

TABLE 84

| | | | Ratio of units (mol %) | | |
|---|---|---|---|---|---|
| A | B | C | A | B | C |
| A-220 | B-3 | C-1 | 50 | 45 | 5 |
| A-221 | B-3 | C-1 | 50 | 45 | 5 |
| A-222 | B-3 | C-1 | 50 | 45 | 5 |
| A-223 | B-3 | C-1 | 50 | 45 | 5 |
| A-224 | B-3 | C-1 | 50 | 45 | 5 |
| A-225 | B-3 | C-1 | 50 | 45 | 5 |
| A-226 | B-3 | C-1 | 50 | 45 | 5 |
| A-227 | B-3 | C-1 | 50 | 45 | 5 |
| A-228 | B-3 | C-1 | 50 | 45 | 5 |
| A-229 | B-3 | C-1 | 50 | 45 | 5 |
| A-230 | B-3 | C-1 | 50 | 45 | 5 |
| A-231 | B-3 | C-1 | 50 | 45 | 5 |
| A-232 | B-3 | C-1 | 50 | 45 | 5 |
| A-233 | B-3 | C-1 | 50 | 45 | 5 |
| A-234 | B-3 | C-1 | 50 | 45 | 5 |
| A-235 | B-3 | C-1 | 50 | 45 | 5 |
| A-236 | B-3 | C-1 | 50 | 45 | 5 |
| A-237 | B-3 | C-1 | 50 | 45 | 5 |
| A-238 | B-3 | C-1 | 50 | 45 | 5 |
| A-239 | B-3 | C-1 | 50 | 45 | 5 |
| A-240 | B-3 | C-1 | 50 | 45 | 5 |

TABLE 85

| | | | Ratio of units (mol %) | | |
|---|---|---|---|---|---|
| A | B | C | A | B | C |
| A-1 | B-3 | C-2 | 50 | 45 | 5 |
| A-2 | B-3 | C-2 | 50 | 45 | 5 |
| A-3 | B-3 | C-2 | 50 | 45 | 5 |
| A-4 | B-3 | C-2 | 50 | 45 | 5 |
| A-5 | B-3 | C-2 | 50 | 45 | 5 |
| A-6 | B-3 | C-2 | 50 | 45 | 5 |
| A-7 | B-3 | C-2 | 50 | 45 | 5 |
| A-8 | B-3 | C-2 | 50 | 45 | 5 |
| A-9 | B-3 | C-2 | 50 | 45 | 5 |
| A-10 | B-3 | C-2 | 50 | 45 | 5 |
| A-11 | B-3 | C-2 | 50 | 45 | 5 |
| A-12 | B-3 | C-2 | 50 | 45 | 5 |
| A-13 | B-3 | C-2 | 50 | 45 | 5 |
| A-14 | B-3 | C-2 | 50 | 45 | 5 |
| A-15 | B-3 | C-2 | 50 | 45 | 5 |
| A-16 | B-3 | C-2 | 50 | 45 | 5 |
| A-17 | B-3 | C-2 | 50 | 45 | 5 |
| A-18 | B-3 | C-2 | 50 | 45 | 5 |
| A-19 | B-3 | C-2 | 50 | 45 | 5 |
| A-20 | B-3 | C-2 | 50 | 45 | 5 |
| A-21 | B-3 | C-2 | 50 | 45 | 5 |
| A-22 | B-3 | C-2 | 50 | 45 | 5 |
| A-23 | B-3 | C-2 | 50 | 45 | 5 |
| A-24 | B-3 | C-2 | 50 | 45 | 5 |
| A-25 | B-3 | C-2 | 50 | 45 | 5 |
| A-26 | B-3 | C-2 | 50 | 45 | 5 |
| A-28 | B-3 | C-2 | 50 | 45 | 5 |
| A-29 | B-3 | C-2 | 50 | 45 | 5 |
| A-30 | B-3 | C-2 | 50 | 45 | 5 |
| A-31 | B-3 | C-2 | 50 | 45 | 5 |
| A-32 | B-3 | C-2 | 50 | 45 | 5 |
| A-33 | B-3 | C-2 | 50 | 45 | 5 |
| A-34 | B-3 | C-2 | 50 | 45 | 5 |
| A-35 | B-3 | C-2 | 50 | 45 | 5 |
| A-36 | B-3 | C-2 | 50 | 45 | 5 |
| A-37 | B-3 | C-2 | 50 | 45 | 5 |
| A-38 | B-3 | C-2 | 50 | 45 | 5 |
| A-39 | B-3 | C-2 | 50 | 45 | 5 |
| A-40 | B-3 | C-2 | 50 | 45 | 5 |
| A-41 | B-3 | C-2 | 50 | 45 | 5 |
| A-42 | B-3 | C-2 | 50 | 45 | 5 |
| A-43 | B-3 | C-2 | 50 | 45 | 5 |
| A-44 | B-3 | C-2 | 50 | 45 | 5 |
| A-45 | B-3 | C-2 | 50 | 45 | 5 |
| A-46 | B-3 | C-2 | 50 | 45 | 5 |
| A-47 | B-3 | C-2 | 50 | 45 | 5 |
| A-49 | B-3 | C-2 | 50 | 45 | 5 |
| A-50 | B-3 | C-2 | 50 | 45 | 5 |
| A-51 | B-3 | C-2 | 50 | 45 | 5 |
| A-52 | B-3 | C-2 | 50 | 45 | 5 |
| A-53 | B-3 | C-2 | 50 | 45 | 5 |
| A-54 | B-3 | C-2 | 50 | 45 | 5 |
| A-55 | B-3 | C-2 | 50 | 45 | 5 |
| A-56 | B-3 | C-2 | 50 | 45 | 5 |
| A-57 | B-3 | C-2 | 50 | 45 | 5 |
| A-58 | B-3 | C-2 | 50 | 45 | 5 |
| A-59 | B-3 | C-2 | 50 | 45 | 5 |
| A-60 | B-3 | C-2 | 50 | 45 | 5 |

TABLE 86

| | | | Ratio of units (mol %) | | |
|---|---|---|---|---|---|
| A | B | C | A | B | C |
| A-61 | B-3 | C-2 | 50 | 45 | 5 |
| A-62 | B-3 | C-2 | 50 | 45 | 5 |
| A-63 | B-3 | C-2 | 50 | 45 | 5 |
| A-64 | B-3 | C-2 | 50 | 45 | 5 |
| A-65 | B-3 | C-2 | 50 | 45 | 5 |
| A-66 | B-3 | C-2 | 50 | 45 | 5 |
| A-67 | B-3 | C-2 | 50 | 45 | 5 |
| A-68 | B-3 | C-2 | 50 | 45 | 5 |
| A-69 | B-3 | C-2 | 50 | 45 | 5 |
| A-70 | B-3 | C-2 | 50 | 45 | 5 |
| A-71 | B-3 | C-2 | 50 | 45 | 5 |
| A-72 | B-3 | C-2 | 50 | 45 | 5 |
| A-73 | B-3 | C-2 | 50 | 45 | 5 |
| A-74 | B-3 | C-2 | 50 | 45 | 5 |
| A-75 | B-3 | C-2 | 50 | 45 | 5 |
| A-76 | B-3 | C-2 | 50 | 45 | 5 |
| A-77 | B-3 | C-2 | 50 | 45 | 5 |
| A-78 | B-3 | C-2 | 50 | 45 | 5 |
| A-79 | B-3 | C-2 | 50 | 45 | 5 |
| A-80 | B-3 | C-2 | 50 | 45 | 5 |
| A-81 | B-3 | C-2 | 50 | 45 | 5 |
| A-82 | B-3 | C-2 | 50 | 45 | 5 |
| A-83 | B-3 | C-2 | 50 | 45 | 5 |
| A-84 | B-3 | C-2 | 50 | 45 | 5 |
| A-85 | B-3 | C-2 | 50 | 45 | 5 |
| A-86 | B-3 | C-2 | 50 | 45 | 5 |
| A-87 | B-3 | C-2 | 50 | 45 | 5 |
| A-88 | B-3 | C-2 | 50 | 45 | 5 |
| A-89 | B-3 | C-2 | 50 | 45 | 5 |
| A-90 | B-3 | C-2 | 50 | 45 | 5 |
| A-91 | B-3 | C-2 | 50 | 45 | 5 |
| A-92 | B-3 | C-2 | 50 | 45 | 5 |
| A-93 | B-3 | C-2 | 50 | 45 | 5 |
| A-94 | B-3 | C-2 | 50 | 45 | 5 |
| A-95 | B-3 | C-2 | 50 | 45 | 5 |
| A-96 | B-3 | C-2 | 50 | 45 | 5 |
| A-100 | B-3 | C-2 | 50 | 45 | 5 |
| A-101 | B-3 | C-2 | 50 | 45 | 5 |
| A-102 | B-3 | C-2 | 50 | 45 | 5 |
| A-103 | B-3 | C-2 | 50 | 45 | 5 |
| A-104 | B-3 | C-2 | 50 | 45 | 5 |
| A-105 | B-3 | C-2 | 50 | 45 | 5 |
| A-106 | B-3 | C-2 | 50 | 45 | 5 |
| A-107 | B-3 | C-2 | 50 | 45 | 5 |
| A-108 | B-3 | C-2 | 50 | 45 | 5 |
| A-109 | B-3 | C-2 | 50 | 45 | 5 |
| A-110 | B-3 | C-2 | 50 | 45 | 5 |
| A-111 | B-3 | C-2 | 50 | 45 | 5 |
| A-112 | B-3 | C-2 | 50 | 45 | 5 |
| A-113 | B-3 | C-2 | 50 | 45 | 5 |
| A-114 | B-3 | C-2 | 50 | 45 | 5 |
| A-115 | B-3 | C-2 | 50 | 45 | 5 |
| A-116 | B-3 | C-2 | 50 | 45 | 5 |
| A-117 | B-3 | C-2 | 50 | 45 | 5 |
| A-118 | B-3 | C-2 | 50 | 45 | 5 |
| A-119 | B-3 | C-2 | 50 | 45 | 5 |
| A-120 | B-3 | C-2 | 50 | 45 | 5 |
| A-121 | B-3 | C-2 | 50 | 45 | 5 |
| A-122 | B-3 | C-2 | 50 | 45 | 5 |
| A-123 | B-3 | C-2 | 50 | 45 | 5 |

TABLE 87

| | | | Ratio of units (mol %) | | |
|---|---|---|---|---|---|
| A | B | C | A | B | C |
| A-124 | B-3 | C-2 | 50 | 45 | 5 |
| A-125 | B-3 | C-2 | 50 | 45 | 5 |
| A-126 | B-3 | C-2 | 50 | 45 | 5 |
| A-127 | B-3 | C-2 | 50 | 45 | 5 |
| A-128 | B-3 | C-2 | 50 | 45 | 5 |
| A-129 | B-3 | C-2 | 50 | 45 | 5 |
| A-130 | B-3 | C-2 | 50 | 45 | 5 |
| A-131 | B-3 | C-2 | 50 | 45 | 5 |
| A-132 | B-3 | C-2 | 50 | 45 | 5 |
| A-133 | B-3 | C-2 | 50 | 45 | 5 |
| A-134 | B-3 | C-2 | 50 | 45 | 5 |
| A-135 | B-3 | C-2 | 50 | 45 | 5 |
| A-136 | B-3 | C-2 | 50 | 45 | 5 |
| A-137 | B-3 | C-2 | 50 | 45 | 5 |
| A-138 | B-3 | C-2 | 50 | 45 | 5 |
| A-139 | B-3 | C-2 | 50 | 45 | 5 |
| A-140 | B-3 | C-2 | 50 | 45 | 5 |
| A-141 | B-3 | C-2 | 50 | 45 | 5 |
| A-142 | B-3 | C-2 | 50 | 45 | 5 |
| A-143 | B-3 | C-2 | 50 | 45 | 5 |
| A-144 | B-3 | C-2 | 50 | 45 | 5 |
| A-145 | B-3 | C-2 | 50 | 45 | 5 |
| A-146 | B-3 | C-2 | 50 | 45 | 5 |
| A-147 | B-3 | C-2 | 50 | 45 | 5 |
| A-160 | B-3 | C-2 | 50 | 45 | 5 |
| A-161 | B-3 | C-2 | 50 | 45 | 5 |
| A-162 | B-3 | C-2 | 50 | 45 | 5 |
| A-163 | B-3 | C-2 | 50 | 45 | 5 |
| A-164 | B-3 | C-2 | 50 | 45 | 5 |
| A-165 | B-3 | C-2 | 50 | 45 | 5 |
| A-166 | B-3 | C-2 | 50 | 45 | 5 |
| A-167 | B-3 | C-2 | 50 | 45 | 5 |
| A-168 | B-3 | C-2 | 50 | 45 | 5 |
| A-169 | B-3 | C-2 | 50 | 45 | 5 |
| A-170 | B-3 | C-2 | 50 | 45 | 5 |
| A-171 | B-3 | C-2 | 50 | 45 | 5 |
| A-172 | B-3 | C-2 | 50 | 45 | 5 |
| A-173 | B-3 | C-2 | 50 | 45 | 5 |
| A-174 | B-3 | C-2 | 50 | 45 | 5 |
| A-175 | B-3 | C-2 | 50 | 45 | 5 |
| A-176 | B-3 | C-2 | 50 | 45 | 5 |
| A-177 | B-3 | C-2 | 50 | 45 | 5 |
| A-178 | B-3 | C-2 | 50 | 45 | 5 |
| A-179 | B-3 | C-2 | 50 | 45 | 5 |
| A-180 | B-3 | C-2 | 50 | 45 | 5 |
| A-181 | B-3 | C-2 | 50 | 45 | 5 |
| A-182 | B-3 | C-2 | 50 | 45 | 5 |
| A-183 | B-3 | C-2 | 50 | 45 | 5 |
| A-190 | B-3 | C-2 | 50 | 45 | 5 |
| A-191 | B-3 | C-2 | 50 | 45 | 5 |
| A-192 | B-3 | C-2 | 50 | 45 | 5 |
| A-193 | B-3 | C-2 | 50 | 45 | 5 |
| A-194 | B-3 | C-2 | 50 | 45 | 5 |
| A-195 | B-3 | C-2 | 50 | 45 | 5 |
| A-196 | B-3 | C-2 | 50 | 45 | 5 |
| A-197 | B-3 | C-2 | 50 | 45 | 5 |
| A-198 | B-3 | C-2 | 50 | 45 | 5 |
| A-199 | B-3 | C-2 | 50 | 45 | 5 |
| A-200 | B-3 | C-2 | 50 | 45 | 5 |
| A-201 | B-3 | C-2 | 50 | 45 | 5 |
| A-202 | B-3 | C-2 | 50 | 45 | 5 |
| A-203 | B-3 | C-2 | 50 | 45 | 5 |
| A-204 | B-3 | C-2 | 50 | 45 | 5 |
| A-205 | B-3 | C-2 | 50 | 45 | 5 |
| A-206 | B-3 | C-2 | 50 | 45 | 5 |
| A-207 | B-3 | C-2 | 50 | 45 | 5 |
| A-208 | B-3 | C-2 | 50 | 45 | 5 |
| A-209 | B-3 | C-2 | 50 | 45 | 5 |
| A-210 | B-3 | C-2 | 50 | 45 | 5 |
| A-211 | B-3 | C-2 | 50 | 45 | 5 |
| A-212 | B-3 | C-2 | 50 | 45 | 5 |
| A-213 | B-3 | C-2 | 50 | 45 | 5 |

TABLE 88

| | | | Ratio of units (mol %) | | |
|---|---|---|---|---|---|
| A | B | C | A | B | C |
| A-220 | B-3 | C-2 | 50 | 45 | 5 |
| A-221 | B-3 | C-2 | 50 | 45 | 5 |
| A-222 | B-3 | C-2 | 50 | 45 | 5 |
| A-223 | B-3 | C-2 | 50 | 45 | 5 |
| A-224 | B-3 | C-2 | 50 | 45 | 5 |
| A-225 | B-3 | C-2 | 50 | 45 | 5 |
| A-226 | B-3 | C-2 | 50 | 45 | 5 |
| A-227 | B-3 | C-2 | 50 | 45 | 5 |
| A-228 | B-3 | C-2 | 50 | 45 | 5 |
| A-229 | B-3 | C-2 | 50 | 45 | 5 |
| A-230 | B-3 | C-2 | 50 | 45 | 5 |
| A-231 | B-3 | C-2 | 50 | 45 | 5 |
| A-232 | B-3 | C-2 | 50 | 45 | 5 |
| A-233 | B-3 | C-2 | 50 | 45 | 5 |
| A-234 | B-3 | C-2 | 50 | 45 | 5 |
| A-235 | B-3 | C-2 | 50 | 45 | 5 |
| A-236 | B-3 | C-2 | 50 | 45 | 5 |
| A-237 | B-3 | C-2 | 50 | 45 | 5 |
| A-238 | B-3 | C-2 | 50 | 45 | 5 |
| A-239 | B-3 | C-2 | 50 | 45 | 5 |
| A-240 | B-3 | C-2 | 50 | 45 | 5 |

TABLE 89

| | | | Ratio of units (mol %) | | |
|---|---|---|---|---|---|
| A | B | C | A | B | C |
| A-1 | B-3 | C-3 | 50 | 45 | 5 |
| A-2 | B-3 | C-3 | 50 | 45 | 5 |
| A-3 | B-3 | C-3 | 50 | 45 | 5 |
| A-4 | B-3 | C-3 | 50 | 45 | 5 |
| A-5 | B-3 | C-3 | 50 | 45 | 5 |
| A-6 | B-3 | C-3 | 50 | 45 | 5 |
| A-7 | B-3 | C-3 | 50 | 45 | 5 |
| A-8 | B-3 | C-3 | 50 | 45 | 5 |
| A-9 | B-3 | C-3 | 50 | 45 | 5 |
| A-10 | B-3 | C-3 | 50 | 45 | 5 |
| A-11 | B-3 | C-3 | 50 | 45 | 5 |
| A-12 | B-3 | C-3 | 50 | 45 | 5 |
| A-13 | B-3 | C-3 | 50 | 45 | 5 |
| A-14 | B-3 | C-3 | 50 | 45 | 5 |
| A-15 | B-3 | C-3 | 50 | 45 | 5 |
| A-16 | B-3 | C-3 | 50 | 45 | 5 |
| A-17 | B-3 | C-3 | 50 | 45 | 5 |
| A-18 | B-3 | C-3 | 50 | 45 | 5 |
| A-19 | B-3 | C-3 | 50 | 45 | 5 |
| A-20 | B-3 | C-3 | 50 | 45 | 5 |
| A-21 | B-3 | C-3 | 50 | 45 | 5 |
| A-22 | B-3 | C-3 | 50 | 45 | 5 |
| A-23 | B-3 | C-3 | 50 | 45 | 5 |
| A-24 | B-3 | C-3 | 50 | 45 | 5 |
| A-25 | B-3 | C-3 | 50 | 45 | 5 |
| A-26 | B-3 | C-3 | 50 | 45 | 5 |
| A-28 | B-3 | C-3 | 50 | 45 | 5 |
| A-29 | B-3 | C-3 | 50 | 45 | 5 |
| A-30 | B-3 | C-3 | 50 | 45 | 5 |
| A-31 | B-3 | C-3 | 50 | 45 | 5 |
| A-32 | B-3 | C-3 | 50 | 45 | 5 |
| A-33 | B-3 | C-3 | 50 | 45 | 5 |
| A-34 | B-3 | C-3 | 50 | 45 | 5 |
| A-35 | B-3 | C-3 | 50 | 45 | 5 |
| A-36 | B-3 | C-3 | 50 | 45 | 5 |
| A-37 | B-3 | C-3 | 50 | 45 | 5 |
| A-38 | B-3 | C-3 | 50 | 45 | 5 |
| A-39 | B-3 | C-3 | 50 | 45 | 5 |
| A-40 | B-3 | C-3 | 50 | 45 | 5 |
| A-41 | B-3 | C-3 | 50 | 45 | 5 |
| A-42 | B-3 | C-3 | 50 | 45 | 5 |
| A-43 | B-3 | C-3 | 50 | 45 | 5 |
| A-44 | B-3 | C-3 | 50 | 45 | 5 |
| A-45 | B-3 | C-3 | 50 | 45 | 5 |
| A-46 | B-3 | C-3 | 50 | 45 | 5 |
| A-47 | B-3 | C-3 | 50 | 45 | 5 |
| A-49 | B-3 | C-3 | 50 | 45 | 5 |
| A-50 | B-3 | C-3 | 50 | 45 | 5 |
| A-51 | B-3 | C-3 | 50 | 45 | 5 |
| A-52 | B-3 | C-3 | 50 | 45 | 5 |
| A-53 | B-3 | C-3 | 50 | 45 | 5 |
| A-54 | B-3 | C-3 | 50 | 45 | 5 |
| A-55 | B-3 | C-3 | 50 | 45 | 5 |
| A-56 | B-3 | C-3 | 50 | 45 | 5 |
| A-57 | B-3 | C-3 | 50 | 45 | 5 |
| A-58 | B-3 | C-3 | 50 | 45 | 5 |
| A-59 | B-3 | C-3 | 50 | 45 | 5 |
| A-60 | B-3 | C-3 | 50 | 45 | 5 |

TABLE 90

| | | | Ratio of units (mol %) | | |
|---|---|---|---|---|---|
| A | B | C | A | B | C |
| A-61 | B-3 | C-3 | 50 | 45 | 5 |
| A-62 | B-3 | C-3 | 50 | 45 | 5 |
| A-63 | B-3 | C-3 | 50 | 45 | 5 |
| A-64 | B-3 | C-3 | 50 | 45 | 5 |
| A-65 | B-3 | C-3 | 50 | 45 | 5 |
| A-66 | B-3 | C-3 | 50 | 45 | 5 |
| A-67 | B-3 | C-3 | 50 | 45 | 5 |
| A-68 | B-3 | C-3 | 50 | 45 | 5 |
| A-69 | B-3 | C-3 | 50 | 45 | 5 |
| A-70 | B-3 | C-3 | 50 | 45 | 5 |
| A-71 | B-3 | C-3 | 50 | 45 | 5 |
| A-72 | B-3 | C-3 | 50 | 45 | 5 |
| A-73 | B-3 | C-3 | 50 | 45 | 5 |
| A-74 | B-3 | C-3 | 50 | 45 | 5 |
| A-75 | B-3 | C-3 | 50 | 45 | 5 |
| A-76 | B-3 | C-3 | 50 | 45 | 5 |
| A-77 | B-3 | C-3 | 50 | 45 | 5 |
| A-78 | B-3 | C-3 | 50 | 45 | 5 |
| A-79 | B-3 | C-3 | 50 | 45 | 5 |
| A-80 | B-3 | C-3 | 50 | 45 | 5 |
| A-81 | B-3 | C-3 | 50 | 45 | 5 |
| A-82 | B-3 | C-3 | 50 | 45 | 5 |
| A-83 | B-3 | C-3 | 50 | 45 | 5 |
| A-84 | B-3 | C-3 | 50 | 45 | 5 |
| A-85 | B-3 | C-3 | 50 | 45 | 5 |
| A-86 | B-3 | C-3 | 50 | 45 | 5 |
| A-87 | B-3 | C-3 | 50 | 45 | 5 |
| A-88 | B-3 | C-3 | 50 | 45 | 5 |
| A-89 | B-3 | C-3 | 50 | 45 | 5 |
| A-90 | B-3 | C-3 | 50 | 45 | 5 |
| A-91 | B-3 | C-3 | 50 | 45 | 5 |
| A-92 | B-3 | C-3 | 50 | 45 | 5 |
| A-93 | B-3 | C-3 | 50 | 45 | 5 |
| A-94 | B-3 | C-3 | 50 | 45 | 5 |
| A-95 | B-3 | C-3 | 50 | 45 | 5 |
| A-96 | B-3 | C-3 | 50 | 45 | 5 |
| A-100 | B-3 | C-3 | 50 | 45 | 5 |
| A-101 | B-3 | C-3 | 50 | 45 | 5 |
| A-102 | B-3 | C-3 | 50 | 45 | 5 |
| A-103 | B-3 | C-3 | 50 | 45 | 5 |
| A-104 | B-3 | C-3 | 50 | 45 | 5 |
| A-105 | B-3 | C-3 | 50 | 45 | 5 |
| A-106 | B-3 | C-3 | 50 | 45 | 5 |
| A-107 | B-3 | C-3 | 50 | 45 | 5 |
| A-108 | B-3 | C-3 | 50 | 45 | 5 |
| A-109 | B-3 | C-3 | 50 | 45 | 5 |
| A-110 | B-3 | C-3 | 50 | 45 | 5 |
| A-111 | B-3 | C-3 | 50 | 45 | 5 |
| A-112 | B-3 | C-3 | 50 | 45 | 5 |
| A-113 | B-3 | C-3 | 50 | 45 | 5 |
| A-114 | B-3 | C-3 | 50 | 45 | 5 |
| A-115 | B-3 | C-3 | 50 | 45 | 5 |
| A-116 | B-3 | C-3 | 50 | 45 | 5 |
| A-117 | B-3 | C-3 | 50 | 45 | 5 |
| A-118 | B-3 | C-3 | 50 | 45 | 5 |
| A-119 | B-3 | C-3 | 50 | 45 | 5 |
| A-120 | B-3 | C-3 | 50 | 45 | 5 |
| A-121 | B-3 | C-3 | 50 | 45 | 5 |
| A-122 | B-3 | C-3 | 50 | 45 | 5 |
| A-123 | B-3 | C-3 | 50 | 45 | 5 |

TABLE 91

| | | | Ratio of units (mol %) | | |
|---|---|---|---|---|---|
| A | B | C | A | B | C |
| A-124 | B-3 | C-3 | 50 | 45 | 5 |
| A-125 | B-3 | C-3 | 50 | 45 | 5 |
| A-126 | B-3 | C-3 | 50 | 45 | 5 |
| A-127 | B-3 | C-3 | 50 | 45 | 5 |
| A-128 | B-3 | C-3 | 50 | 45 | 5 |
| A-129 | B-3 | C-3 | 50 | 45 | 5 |
| A-130 | B-3 | C-3 | 50 | 45 | 5 |
| A-131 | B-3 | C-3 | 50 | 45 | 5 |
| A-132 | B-3 | C-3 | 50 | 45 | 5 |
| A-133 | B-3 | C-3 | 50 | 45 | 5 |
| A-134 | B-3 | C-3 | 50 | 45 | 5 |
| A-135 | B-3 | C-3 | 50 | 45 | 5 |
| A-136 | B-3 | C-3 | 50 | 45 | 5 |
| A-137 | B-3 | C-3 | 50 | 45 | 5 |
| A-138 | B-3 | C-3 | 50 | 45 | 5 |
| A-139 | B-3 | C-3 | 50 | 45 | 5 |
| A-140 | B-3 | C-3 | 50 | 45 | 5 |
| A-141 | B-3 | C-3 | 50 | 45 | 5 |
| A-142 | B-3 | C-3 | 50 | 45 | 5 |
| A-143 | B-3 | C-3 | 50 | 45 | 5 |
| A-144 | B-3 | C-3 | 50 | 45 | 5 |
| A-145 | B-3 | C-3 | 50 | 45 | 5 |
| A-146 | B-3 | C-3 | 50 | 45 | 5 |
| A-147 | B-3 | C-3 | 50 | 45 | 5 |
| A-160 | B-3 | C-3 | 50 | 45 | 5 |
| A-161 | B-3 | C-3 | 50 | 45 | 5 |
| A-162 | B-3 | C-3 | 50 | 45 | 5 |
| A-163 | B-3 | C-3 | 50 | 45 | 5 |
| A-164 | B-3 | C-3 | 50 | 45 | 5 |
| A-165 | B-3 | C-3 | 50 | 45 | 5 |
| A-166 | B-3 | C-3 | 50 | 45 | 5 |
| A-167 | B-3 | C-3 | 50 | 45 | 5 |
| A-168 | B-3 | C-3 | 50 | 45 | 5 |
| A-169 | B-3 | C-3 | 50 | 45 | 5 |
| A-170 | B-3 | C-3 | 50 | 45 | 5 |
| A-171 | B-3 | C-3 | 50 | 45 | 5 |
| A-172 | B-3 | C-3 | 50 | 45 | 5 |
| A-173 | B-3 | C-3 | 50 | 45 | 5 |
| A-174 | B-3 | C-3 | 50 | 45 | 5 |
| A-175 | B-3 | C-3 | 50 | 45 | 5 |
| A-176 | B-3 | C-3 | 50 | 45 | 5 |
| A-177 | B-3 | C-3 | 50 | 45 | 5 |
| A-178 | B-3 | C-3 | 50 | 45 | 5 |
| A-179 | B-3 | C-3 | 50 | 45 | 5 |
| A-180 | B-3 | C-3 | 50 | 45 | 5 |
| A-181 | B-3 | C-3 | 50 | 45 | 5 |
| A-182 | B-3 | C-3 | 50 | 45 | 5 |
| A-183 | B-3 | C-3 | 50 | 45 | 5 |
| A-190 | B-3 | C-3 | 50 | 45 | 5 |
| A-191 | B-3 | C-3 | 50 | 45 | 5 |
| A-192 | B-3 | C-3 | 50 | 45 | 5 |
| A-193 | B-3 | C-3 | 50 | 45 | 5 |
| A-194 | B-3 | C-3 | 50 | 45 | 5 |
| A-195 | B-3 | C-3 | 50 | 45 | 5 |
| A-196 | B-3 | C-3 | 50 | 45 | 5 |
| A-197 | B-3 | C-3 | 50 | 45 | 5 |
| A-198 | B-3 | C-3 | 50 | 45 | 5 |
| A-199 | B-3 | C-3 | 50 | 45 | 5 |
| A-200 | B-3 | C-3 | 50 | 45 | 5 |
| A-201 | B-3 | C-3 | 50 | 45 | 5 |
| A-202 | B-3 | C-3 | 50 | 45 | 5 |
| A-203 | B-3 | C-3 | 50 | 45 | 5 |
| A-204 | B-3 | C-3 | 50 | 45 | 5 |
| A-205 | B-3 | C-3 | 50 | 45 | 5 |
| A-206 | B-3 | C-3 | 50 | 45 | 5 |
| A-207 | B-3 | C-3 | 50 | 45 | 5 |
| A-208 | B-3 | C-3 | 50 | 45 | 5 |
| A-209 | B-3 | C-3 | 50 | 45 | 5 |
| A-210 | B-3 | C-3 | 50 | 45 | 5 |
| A-211 | B-3 | C-3 | 50 | 45 | 5 |
| A-212 | B-3 | C-3 | 50 | 45 | 5 |
| A-213 | B-3 | C-3 | 50 | 45 | 5 |

TABLE 92

| | | | Ratio of units (mol %) | | |
|---|---|---|---|---|---|
| A | B | C | A | B | C |
| A-220 | B-3 | C-3 | 50 | 45 | 5 |
| A-221 | B-3 | C-3 | 50 | 45 | 5 |
| A-222 | B-3 | C-3 | 50 | 45 | 5 |
| A-223 | B-3 | C-3 | 50 | 45 | 5 |
| A-224 | B-3 | C-3 | 50 | 45 | 5 |
| A-225 | B-3 | C-3 | 50 | 45 | 5 |
| A-226 | B-3 | C-3 | 50 | 45 | 5 |
| A-227 | B-3 | C-3 | 50 | 45 | 5 |
| A-228 | B-3 | C-3 | 50 | 45 | 5 |
| A-229 | B-3 | C-3 | 50 | 45 | 5 |
| A-230 | B-3 | C-3 | 50 | 45 | 5 |
| A-231 | B-3 | C-3 | 50 | 45 | 5 |
| A-232 | B-3 | C-3 | 50 | 45 | 5 |
| A-233 | B-3 | C-3 | 50 | 45 | 5 |
| A-234 | B-3 | C-3 | 50 | 45 | 5 |
| A-235 | B-3 | C-3 | 50 | 45 | 5 |
| A-236 | B-3 | C-3 | 50 | 45 | 5 |
| A-237 | B-3 | C-3 | 50 | 45 | 5 |
| A-238 | B-3 | C-3 | 50 | 45 | 5 |
| A-239 | B-3 | C-3 | 50 | 45 | 5 |
| A-240 | B-3 | C-3 | 50 | 45 | 5 |

TABLE 93

| | | | Ratio of units (mol %) | | |
|---|---|---|---|---|---|
| A | B | C | A | B | C |
| A-1 | B-3 | C-4 | 50 | 45 | 5 |
| A-2 | B-3 | C-4 | 50 | 45 | 5 |
| A-3 | B-3 | C-4 | 50 | 45 | 5 |
| A-4 | B-3 | C-4 | 50 | 45 | 5 |
| A-5 | B-3 | C-4 | 50 | 45 | 5 |
| A-6 | B-3 | C-4 | 50 | 45 | 5 |
| A-7 | B-3 | C-4 | 50 | 45 | 5 |
| A-8 | B-3 | C-4 | 50 | 45 | 5 |
| A-9 | B-3 | C-4 | 50 | 45 | 5 |
| A-10 | B-3 | C-4 | 50 | 45 | 5 |
| A-11 | B-3 | C-4 | 50 | 45 | 5 |
| A-12 | B-3 | C-4 | 50 | 45 | 5 |
| A-13 | B-3 | C-4 | 50 | 45 | 5 |
| A-14 | B-3 | C-4 | 50 | 45 | 5 |
| A-15 | B-3 | C-4 | 50 | 45 | 5 |
| A-16 | B-3 | C-4 | 50 | 45 | 5 |
| A-17 | B-3 | C-4 | 50 | 45 | 5 |
| A-18 | B-3 | C-4 | 50 | 45 | 5 |
| A-19 | B-3 | C-4 | 50 | 45 | 5 |
| A-20 | B-3 | C-4 | 50 | 45 | 5 |
| A-21 | B-3 | C-4 | 50 | 45 | 5 |
| A-22 | B-3 | C-4 | 50 | 45 | 5 |
| A-23 | B-3 | C-4 | 50 | 45 | 5 |
| A-24 | B-3 | C-4 | 50 | 45 | 5 |
| A-25 | B-3 | C-4 | 50 | 45 | 5 |
| A-26 | B-3 | C-4 | 50 | 45 | 5 |
| A-28 | B-3 | C-4 | 50 | 45 | 5 |
| A-29 | B-3 | C-4 | 50 | 45 | 5 |
| A-30 | B-3 | C-4 | 50 | 45 | 5 |
| A-31 | B-3 | C-4 | 50 | 45 | 5 |
| A-32 | B-3 | C-4 | 50 | 45 | 5 |
| A-33 | B-3 | C-4 | 50 | 45 | 5 |
| A-34 | B-3 | C-4 | 50 | 45 | 5 |
| A-35 | B-3 | C-4 | 50 | 45 | 5 |
| A-36 | B-3 | C-4 | 50 | 45 | 5 |
| A-37 | B-3 | C-4 | 50 | 45 | 5 |
| A-38 | B-3 | C-4 | 50 | 45 | 5 |
| A-39 | B-3 | C-4 | 50 | 45 | 5 |
| A-40 | B-3 | C-4 | 50 | 45 | 5 |
| A-41 | B-3 | C-4 | 50 | 45 | 5 |
| A-42 | B-3 | C-4 | 50 | 45 | 5 |
| A-43 | B-3 | C-4 | 50 | 45 | 5 |
| A-44 | B-3 | C-4 | 50 | 45 | 5 |
| A-45 | B-3 | C-4 | 50 | 45 | 5 |
| A-46 | B-3 | C-4 | 50 | 45 | 5 |
| A-47 | B-3 | C-4 | 50 | 45 | 5 |
| A-49 | B-3 | C-4 | 50 | 45 | 5 |
| A-50 | B-3 | C-4 | 50 | 45 | 5 |
| A-51 | B-3 | C-4 | 50 | 45 | 5 |
| A-52 | B-3 | C-4 | 50 | 45 | 5 |
| A-53 | B-3 | C-4 | 50 | 45 | 5 |
| A-54 | B-3 | C-4 | 50 | 45 | 5 |
| A-55 | B-3 | C-4 | 50 | 45 | 5 |
| A-56 | B-3 | C-4 | 50 | 45 | 5 |
| A-57 | B-3 | C-4 | 50 | 45 | 5 |
| A-58 | B-3 | C-4 | 50 | 45 | 5 |
| A-59 | B-3 | C-4 | 50 | 45 | 5 |
| A-60 | B-3 | C-4 | 50 | 45 | 5 |

TABLE 94

| | | | Ratio of units (mol %) | | |
|---|---|---|---|---|---|
| A | B | C | A | B | C |
| A-61 | B-3 | C-4 | 50 | 45 | 5 |
| A-62 | B-3 | C-4 | 50 | 45 | 5 |
| A-63 | B-3 | C-4 | 50 | 45 | 5 |
| A-64 | B-3 | C-4 | 50 | 45 | 5 |
| A-65 | B-3 | C-4 | 50 | 45 | 5 |
| A-66 | B-3 | C-4 | 50 | 45 | 5 |
| A-67 | B-3 | C-4 | 50 | 45 | 5 |
| A-68 | B-3 | C-4 | 50 | 45 | 5 |
| A-69 | B-3 | C-4 | 50 | 45 | 5 |
| A-70 | B-3 | C-4 | 50 | 45 | 5 |
| A-71 | B-3 | C-4 | 50 | 45 | 5 |
| A-72 | B-3 | C-4 | 50 | 45 | 5 |
| A-73 | B-3 | C-4 | 50 | 45 | 5 |
| A-74 | B-3 | C-4 | 50 | 45 | 5 |
| A-75 | B-3 | C-4 | 50 | 45 | 5 |
| A-76 | B-3 | C-4 | 50 | 45 | 5 |
| A-77 | B-3 | C-4 | 50 | 45 | 5 |
| A-78 | B-3 | C-4 | 50 | 45 | 5 |
| A-79 | B-3 | C-4 | 50 | 45 | 5 |
| A-80 | B-3 | C-4 | 50 | 45 | 5 |
| A-81 | B-3 | C-4 | 50 | 45 | 5 |
| A-82 | B-3 | C-4 | 50 | 45 | 5 |
| A-83 | B-3 | C-4 | 50 | 45 | 5 |
| A-84 | B-3 | C-4 | 50 | 45 | 5 |
| A-85 | B-3 | C-4 | 50 | 45 | 5 |
| A-86 | B-3 | C-4 | 50 | 45 | 5 |
| A-87 | B-3 | C-4 | 50 | 45 | 5 |
| A-88 | B-3 | C-4 | 50 | 45 | 5 |
| A-89 | B-3 | C-4 | 50 | 45 | 5 |
| A-90 | B-3 | C-4 | 50 | 45 | 5 |
| A-91 | B-3 | C-4 | 50 | 45 | 5 |
| A-92 | B-3 | C-4 | 50 | 45 | 5 |
| A-93 | B-3 | C-4 | 50 | 45 | 5 |
| A-94 | B-3 | C-4 | 50 | 45 | 5 |
| A-95 | B-3 | C-4 | 50 | 45 | 5 |
| A-96 | B-3 | C-4 | 50 | 45 | 5 |
| A-100 | B-3 | C-4 | 50 | 45 | 5 |
| A-101 | B-3 | C-4 | 50 | 45 | 5 |
| A-102 | B-3 | C-4 | 50 | 45 | 5 |
| A-103 | B-3 | C-4 | 50 | 45 | 5 |
| A-104 | B-3 | C-4 | 50 | 45 | 5 |
| A-105 | B-3 | C-4 | 50 | 45 | 5 |
| A-106 | B-3 | C-4 | 50 | 45 | 5 |
| A-107 | B-3 | C-4 | 50 | 45 | 5 |
| A-108 | B-3 | C-4 | 50 | 45 | 5 |
| A-109 | B-3 | C-4 | 50 | 45 | 5 |
| A-110 | B-3 | C-4 | 50 | 45 | 5 |
| A-111 | B-3 | C-4 | 50 | 45 | 5 |
| A-112 | B-3 | C-4 | 50 | 45 | 5 |
| A-113 | B-3 | C-4 | 50 | 45 | 5 |
| A-114 | B-3 | C-4 | 50 | 45 | 5 |
| A-115 | B-3 | C-4 | 50 | 45 | 5 |
| A-116 | B-3 | C-4 | 50 | 45 | 5 |
| A-117 | B-3 | C-4 | 50 | 45 | 5 |
| A-118 | B-3 | C-4 | 50 | 45 | 5 |
| A-119 | B-3 | C-4 | 50 | 45 | 5 |
| A-120 | B-3 | C-4 | 50 | 45 | 5 |
| A-121 | B-3 | C-4 | 50 | 45 | 5 |
| A-122 | B-3 | C-4 | 50 | 45 | 5 |
| A-123 | B-3 | C-4 | 50 | 45 | 5 |

TABLE 95

| | | | Ratio of units (mol %) | | |
|---|---|---|---|---|---|
| A | B | C | A | B | C |
| A-124 | B-3 | C-4 | 50 | 45 | 5 |
| A-125 | B-3 | C-4 | 50 | 45 | 5 |
| A-126 | B-3 | C-4 | 50 | 45 | 5 |
| A-127 | B-3 | C-4 | 50 | 45 | 5 |
| A-128 | B-3 | C-4 | 50 | 45 | 5 |
| A-129 | B-3 | C-4 | 50 | 45 | 5 |
| A-130 | B-3 | C-4 | 50 | 45 | 5 |
| A-131 | B-3 | C-4 | 50 | 45 | 5 |
| A-132 | B-3 | C-4 | 50 | 45 | 5 |
| A-133 | B-3 | C-4 | 50 | 45 | 5 |
| A-134 | B-3 | C-4 | 50 | 45 | 5 |
| A-135 | B-3 | C-4 | 50 | 45 | 5 |
| A-136 | B-3 | C-4 | 50 | 45 | 5 |
| A-137 | B-3 | C-4 | 50 | 45 | 5 |
| A-138 | B-3 | C-4 | 50 | 45 | 5 |
| A-139 | B-3 | C-4 | 50 | 45 | 5 |
| A-140 | B-3 | C-4 | 50 | 45 | 5 |
| A-141 | B-3 | C-4 | 50 | 45 | 5 |
| A-142 | B-3 | C-4 | 50 | 45 | 5 |
| A-143 | B-3 | C-4 | 50 | 45 | 5 |
| A-144 | B-3 | C-4 | 50 | 45 | 5 |
| A-145 | B-3 | C-4 | 50 | 45 | 5 |
| A-146 | B-3 | C-4 | 50 | 45 | 5 |
| A-147 | B-3 | C-4 | 50 | 45 | 5 |
| A-160 | B-3 | C-4 | 50 | 45 | 5 |
| A-161 | B-3 | C-4 | 50 | 45 | 5 |
| A-162 | B-3 | C-4 | 50 | 45 | 5 |
| A-163 | B-3 | C-4 | 50 | 45 | 5 |
| A-164 | B-3 | C-4 | 50 | 45 | 5 |
| A-165 | B-3 | C-4 | 50 | 45 | 5 |
| A-166 | B-3 | C-4 | 50 | 45 | 5 |
| A-167 | B-3 | C-4 | 50 | 45 | 5 |
| A-168 | B-3 | C-4 | 50 | 45 | 5 |
| A-169 | B-3 | C-4 | 50 | 45 | 5 |
| A-170 | B-3 | C-4 | 50 | 45 | 5 |
| A-171 | B-3 | C-4 | 50 | 45 | 5 |
| A-172 | B-3 | C-4 | 50 | 45 | 5 |
| A-173 | B-3 | C-4 | 50 | 45 | 5 |
| A-174 | B-3 | C-4 | 50 | 45 | 5 |
| A-175 | B-3 | C-4 | 50 | 45 | 5 |
| A-176 | B-3 | C-4 | 50 | 45 | 5 |
| A-177 | B-3 | C-4 | 50 | 45 | 5 |
| A-178 | B-3 | C-4 | 50 | 45 | 5 |
| A-179 | B-3 | C-4 | 50 | 45 | 5 |
| A-180 | B-3 | C-4 | 50 | 45 | 5 |
| A-181 | B-3 | C-4 | 50 | 45 | 5 |
| A-182 | B-3 | C-4 | 50 | 45 | 5 |
| A-183 | B-3 | C-4 | 50 | 45 | 5 |
| A-190 | B-3 | C-4 | 50 | 45 | 5 |
| A-191 | B-3 | C-4 | 50 | 45 | 5 |
| A-192 | B-3 | C-4 | 50 | 45 | 5 |
| A-193 | B-3 | C-4 | 50 | 45 | 5 |
| A-194 | B-3 | C-4 | 50 | 45 | 5 |
| A-195 | B-3 | C-4 | 50 | 45 | 5 |
| A-196 | B-3 | C-4 | 50 | 45 | 5 |
| A-197 | B-3 | C-4 | 50 | 45 | 5 |
| A-198 | B-3 | C-4 | 50 | 45 | 5 |
| A-199 | B-3 | C-4 | 50 | 45 | 5 |
| A-200 | B-3 | C-4 | 50 | 45 | 5 |
| A-201 | B-3 | C-4 | 50 | 45 | 5 |
| A-202 | B-3 | C-4 | 50 | 45 | 5 |
| A-203 | B-3 | C-4 | 50 | 45 | 5 |
| A-204 | B-3 | C-4 | 50 | 45 | 5 |
| A-205 | B-3 | C-4 | 50 | 45 | 5 |
| A-206 | B-3 | C-4 | 50 | 45 | 5 |
| A-207 | B-3 | C-4 | 50 | 45 | 5 |
| A-208 | B-3 | C-4 | 50 | 45 | 5 |
| A-209 | B-3 | C-4 | 50 | 45 | 5 |
| A-210 | B-3 | C-4 | 50 | 45 | 5 |
| A-211 | B-3 | C-4 | 50 | 45 | 5 |
| A-212 | B-3 | C-4 | 50 | 45 | 5 |
| A-213 | B-3 | C-4 | 50 | 45 | 5 |

TABLE 96

| | | | Ratio of units (mol %) | | |
|---|---|---|---|---|---|
| A | B | C | A | B | C |
| A-220 | B-3 | C-4 | 50 | 45 | 5 |
| A-221 | B-3 | C-4 | 50 | 45 | 5 |
| A-222 | B-3 | C-4 | 50 | 45 | 5 |
| A-223 | B-3 | C-4 | 50 | 45 | 5 |
| A-224 | B-3 | C-4 | 50 | 45 | 5 |
| A-225 | B-3 | C-4 | 50 | 45 | 5 |
| A-226 | B-3 | C-4 | 50 | 45 | 5 |
| A-227 | B-3 | C-4 | 50 | 45 | 5 |
| A-228 | B-3 | C-4 | 50 | 45 | 5 |
| A-229 | B-3 | C-4 | 50 | 45 | 5 |
| A-230 | B-3 | C-4 | 50 | 45 | 5 |
| A-231 | B-3 | C-4 | 50 | 45 | 5 |
| A-232 | B-3 | C-4 | 50 | 45 | 5 |
| A-233 | B-3 | C-4 | 50 | 45 | 5 |
| A-234 | B-3 | C-4 | 50 | 45 | 5 |
| A-235 | B-3 | C-4 | 50 | 45 | 5 |
| A-236 | B-3 | C-4 | 50 | 45 | 5 |
| A-237 | B-3 | C-4 | 50 | 45 | 5 |
| A-238 | B-3 | C-4 | 50 | 45 | 5 |
| A-239 | B-3 | C-4 | 50 | 45 | 5 |
| A-240 | B-3 | C-4 | 50 | 45 | 5 |

In the cross-conjugated polymer, the molar ratio (M_{A}) of the structural unit (A) and the molar ratio (M_{B}) of the structural unit (B) are preferably in the relationship represented by formula (1):

M_{A}≥M_{B} (1)

and more preferably in the relationship represented by formula (2):

M_{A}>M_{B} (2).

The molar ratio (M_{A}) of the structural unit (A) is preferably 50 mol % or more, more preferably more than 50 mol %, further more preferably 60 mol % or more, and still further more preferably 80 mol % or more, each based on the whole copolymer.

The molar ratio (M_{B}) of the structural unit (B) is preferably 50 mol % or less, more preferably less than 50 mol %, further more preferably 45 mol % or less, and still further more preferably 40 mol % or less, each based on the whole copolymer. The molar ratio (M_{B}) is preferably 10 mol % or more, more preferably 20 mol % or more, and further more preferably 30 mol % or more.

In the cross-conjugated polymer, the molar ratio (M_{A}) of the structural unit (A), the molar ratio (M_{B}) of the structural unit (B), and the molar ratio (M_{C}) of the structural unit (C) are preferably in the relationship represented by formula (3):

M_{A}≥M_{B}+M_{C} (3).

The molar ratio (M_{C}) of the structural unit (C) is preferably more than 0 mol %, more preferably 1 mol % or more, further more preferably 5 mol % or more, and still further more preferably 10 mol % or more, each based on the whole copolymer. The molar ratio (M_{C}) is preferably 30 mol % or less, and more preferably 20 mol % or less.

In view of obtaining a viscosity suitable for a coating method, the weight average molecular weight Mw of the cross-conjugated polymer is preferably 10,000 to 1,500,000 and more preferably 15,000 to 1,000,000.

The number average molecular weight Mn of the copolymer is preferably 1,000 to 5,000,000, more preferably 3,000 to 500,000, and further more preferably 5,000 to 100,000.

The molecular weight distribution represented by Mw/Mn, wherein Mw is a weight average molecular weight and Mn is a number average molecular weight is preferably small, although not particularly limited. The molecular weight distribution (Mw/Mn) is preferably 1.0 to 20, more preferably 1.2 to 18, and further more preferably 1.5 to 15.

The number average molecular weight and the weight average molecular weight can be determined by size-exclusion chromatography (SEC) employing the calibration with a standard polystyrene.

The polymer of the invention can be produced by cross-coupling the monomers corresponding to the structural unit (A), the structural unit (B), and the structural unit (C) using a general cross-coupling reaction, such as Suzuki coupling reaction.

A palladium compound, such as a Pd(II) salt, preferably palladium acetate, or a Pd(0) complex, preferably Pd(Ph₃P)₄ wherein Ph is a phenyl group, may be used as the polymerization catalyst. When a Pd(II) salt is used, it is advantageous to add a phosphine derivative into the reaction mixture in an amount of 2 to 8 molar equivalent per one mole of the Pd salt. A Pd(II)-Ph₃P complex, such as PdCl₂(Ph₃P)₂, is also usable. The amount of Pd per one mole of a halogen-containing functional monomer, for example, a monomer wherein a halogen atom is bonded to the bonding site of the structural unit (A), the structural unit (B), or the structural unit (C), is preferably 1×10⁻⁶ to 1×10⁻² mol, more preferably 1×10⁻⁵ to 1×10⁻² mol, and most preferably 1×10⁻⁴ to 5×10⁻³ mol. Within the above ranges, the production of a polymer having a weight average molecular weight of 50,000 to 1,500,000 may be ensured.

In the polymerization by Suzuki coupling reaction, a base, such as cesium carbonate, is generally used. The amount of the base to a boronic acid functional monomer, for example, a monomer wherein boronic acid is bonded to the bonding site of the structural unit (A), the structural unit (B), or the structural unit (C), is preferably 0.5 to 2.0 mol per one molar equivalent of boronic acid.

Examples of the polymerization solvent include, but not particularly limited to, an aromatic hydrocarbon solvent, an aliphatic hydrocarbon solvent, an ether solvent, N-methylpyrrolidone, N, N-dimethylformamide, N, N-dimethylacetamide, dimethyl sulfoxide, and acetonitrile. These may be used alone or in combination.

Examples of the aromatic hydrocarbon solvent include benzene, toluene, xylene, and mesitylene; examples of the aliphatic hydrocarbon solvent include hexane, heptane, cyclohexane, methylcyclohexane, and ethylcyclohexane; and examples of the ether solvent include diethyl ether, diisopropyl ether, ethylene glycol dimethyl ether, diethylene glycol dimethyl ether, 1,4-dioxane, and tetrahydrofuran.

A mixed solvent of an aromatic hydrocarbon solvent, such as toluene, with an ether solvent, such as 1, 4-dioxane, is preferable, because it ensures the production of a polymer having a weight average molecular weight of 50,000 to 1,500,000.

The polymerization pressure is preferably 0.01 to 10 MPa and particularly preferably ambient pressure to 1 MPa, both in terms of absolute pressure. If being excessively high, a special apparatus is needed for working safety, this reducing the industrial productivity.

The polymerization temperature is preferably 0 to 200° C. and particularly preferably 50 to 150° C. If being excessively low, the reaction speed is decreased to prolong the reaction time. If being excessively high, the catalyst is easily deactivated and the desired polymer is not obtained.

The representative polymerization of the invention is illustrated below.

The following is an example of the production of the cross-conjugated polymer.

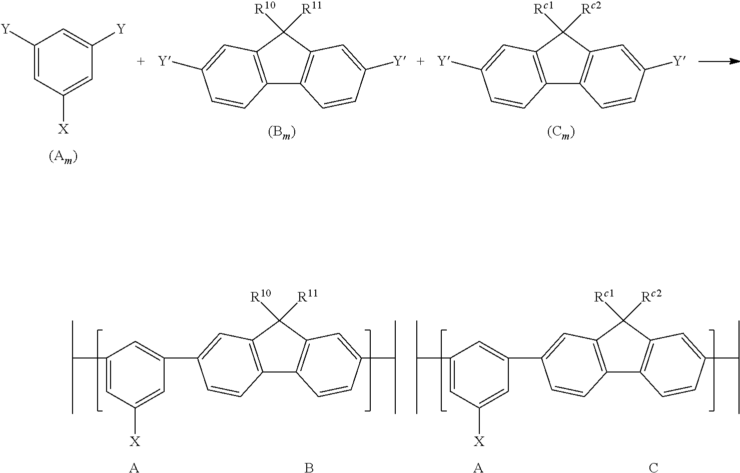

In the above, R¹⁰, R¹¹, R^{c1}, and R^{c2} are the same as R mentioned above. When Y is I, Br, Cl, or OSO₂(C₆H₄)CH₃ and Y′ is boronic acid or a boronic ester, or when Y is boronic acid or a boronic ester and Y′ is I, Br, Cl, or OSO₂(C₆H₄)CH₃, an alternating polymer is obtained. The molar equivalent of Y is preferably the same as the molar equivalent of Y′. The ratio of the structural unit A-B and the structural unit A-C can be controlled by changing the charged amounts of the starting monomer Am and the starting monomer Cm.

When a random copolymer is intended, the starting monomers are selected as shown below.

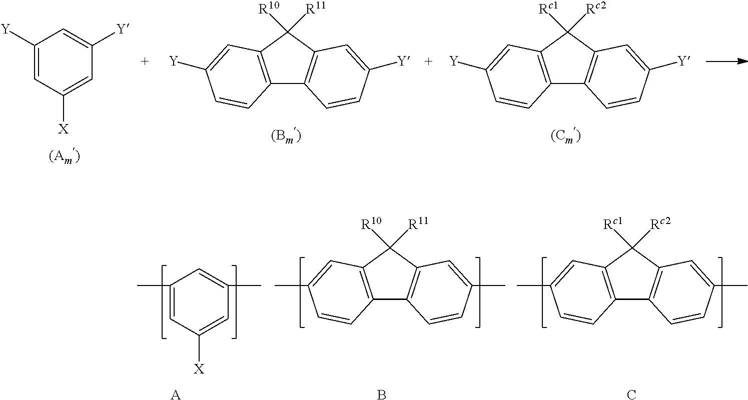

In the above, R¹⁰, R¹¹, R^{c1}, and R^{c2} are the same as described above. Y is I, Br, Cl, or OSO₂(C₆H₄)CH₃, and Y′ is boronic acid or a boronic ester. For example, by using the monomers (Am′), (Bm′), and (Cm′), the units can be arranged randomly.

In the present invention, a dispersion or a solution in which at least one polymer is dispersed or dissolved in a solution is preferably used.

An antioxidant which does not adversely affect the performance of the organic EL device, such as a phosphorus-containing antioxidant, may be added in the thin film-forming process. In addition, other compounds, such as an acceptor material, may be added depending upon the use.

The polymer of the invention is useful as a material for electronic device and a material for organic electroluminescence device. Examples of the electronic device other than the organic electroluminescence device include an organic thin film solar cell and an organic thin film transistor. The polymer of the invention is particularly suitable as a material for organic electroluminescence device, such as a material for use in a light emitting layer or a hole transporting zone (a hole transporting layer, a hole injecting layer, etc.).

The organic EL device of the invention comprises an anode, a cathode, and an organic thin film layer and at least one layer of the organic thin film layer comprises the polymer of the invention. The method of producing the organic EL device of the invention comprises forming a coating solution comprising the polymer of the invention into a thin film layer by a coating method.

The organic thin film layer comprising the polymer of the invention is preferably a hole transporting zone. The layer to be formed in the hole transporting zone may include a hole transporting layer and a hole injecting layer. The hole transporting layer or the hole injecting layer is preferably in contact with the light emitting layer.

In the present invention, the polymer is included in at least one layer selected from the hole transporting layer and the hole injecting layer more preferably as a main component. The content of the polymer of the invention in the hole transporting layer or the hole injecting layer is preferably 51 to 100% by mass.

The following (1) to (13) are representative device structures for the organic EL device of the invention:

(1) Anode/Light emitting layer/Cathode;
(2) Anode/Hole injecting layer/Light emitting layer/Cathode;
(3) Anode/Light emitting layer/Electron injecting layer/Cathode;
(4) Anode/Hole injecting layer/Light emitting layer/Electron injecting layer/Cathode;
(5) Anode/Organic semiconductor layer/Light emitting layer/Cathode;
(6) Anode/Organic semiconductor layer/Electron blocking layer/light emitting layer/Cathode;
(7) Anode/Organic semiconductor layer/Light emitting layer/Adhesion improving layer/Cathode;
(8) Anode/Hole injecting layer/Hole transporting layer/Light emitting layer/Electron injecting layer/Cathode;
(9) Anode/Insulating layer/Light emitting layer/Insulating layer/Cathode;
(10) Anode/Inorganic semiconductor layer/Insulating layer/Light emitting layer/Insulating layer/Cathode;
(11) Anode/Organic semiconductor layer/Insulating layer/Light emitting layer/Insulating layer/Cathode;
(12) Anode/Insulating layer/Hole injecting layer/Hole transporting layer/Light emitting layer/Insulating layer/Cathode; and
(13) Anode/Insulating layer/Hole injecting layer/Hole transporting layer/Light emitting layer/Electron injecting layer/Cathode.

Of the above, the device structure (8) is preferably used, although not limited thereto.

Each layer of the organic EL device may be formed by a known method, such as a vacuum vapor deposition method and a spin coating method, for example, by a vacuum vapor deposition method, a molecular beam epitaxy method (MBE method) or a coating method using a solution of the compound for forming the layer, such as a dipping method, a spin coating method, a casting method, a bar coating method and a roll coating method.

The thickness of each organic layer of the organic EL device is not particularly limited and generally 1 nm to 15 μm, preferably 5 nm to 10 μm, more preferably 5 nm to 1 μm, and further more preferably 5 nm to 0.2 μm, because an excessively small thickness may cause defects, such as pin holes, and an excessively large thickness may require a high applied voltage to reduce the efficiency.

The layer comprising the polymer of the invention (a hole transporting layer and a hole injecting layer) is preferably formed by the above coating method using a solution comprising a solvent and the polymer (ink composition).

The coating method is preferably a wet film-forming method, for example, a letterpress printing method, an intaglio printing method, a lithographic printing method, a stencil printing method, a combination of the preceding methods with an offset printing method, an inkjet printing method, a dispenser coating method, a spin coating method, a bar coating method, a dip coating method, a spray coating method, a slit coating method, a roll coating method, a cap coating method, a rotogravure roll coating method, and a meniscus coating method. If a fine patterning is required, a letterpress printing method, an intaglio printing method, a lithographic printing method, a stencil printing method, a combination of the preceding methods with an offset printing method, an inkjet printing method, and a dispenser coating method are preferred. A transfer coating method is also usable, in which the polymer is preformed into a film on a substrate by the wet film-foaming method described above and then the preformed film is transferred onto a substrate having an electrode printed thereon by a laser light or hot press. The film formation by the above methods can be made under the conditions well known to a person skilled in the art, therefore the details thereof is omitted.

The coating solution for use in the coating method (ink composition) may include at least one polymer of the invention, which may be dissolved or dispersed in a solvent. The content of the polymer in the coating solution (ink composition) is preferably 0.1 to 15% by mass and more preferably 0.5 to 10% by mass based on the total of the film-forming solution.

The solvent is preferably an organic solvent, for example, a chlorine-containing solvent, such as chloroform, chlorobenzene, chlorotoluene, chloroxylene, chloroanisole, dichloromethane, dichlorobenzene, dichlorotoluene, dichloroethane, trichloroethane, trichlorobenzene, trichloromethylbenzene, bromobenzene, dibromobenzene, and bromoanisole; an ether solvent, such as tetrahydrofuran, dioxane, dioxolane, oxazole, methylbenzoxazole, benzisooxazole, furan, furazan, benzofuran, and dihydrobenzofuran; an aromatic hydrocarbon solvent, such as ethylbenzene, diethylbenzene, triethylbenzene, trimethylbenzene, trimethoxybenzene, propylbenzene, isopropylbenzene, diisopropylbenzene, dibutylbenzene, amylbenzene, dihexylbenzene, cyclohexylbenzene, tetramethylbenzene, dodecylbenzene, benzonitrile, acetophenone, methylacetophenone, methoxyacetophenone, ethyl toluate, toluene, ethyltoluene, methoxytoluene, dimethoxytoluene, trimethoxytoluene, isopropyltoluene, xylene, butylxylene, isopropylxylene, anisole, ethylanisole, dimethylanisole, trimethylanisole, propylanisole, isopropylanisole, butylanisole, methylethylanisole, anethole, anisyl alcohol, methyl benzoate, ethyl benzoate, propyl benzoate, butyl benzoate, diphenyl ether, butyl phenyl ether, benzyl methyl ether, benzyl ethyl ether, methylenedioxybenzene, methylnaphthalene, tetrahydronaphthalene, aniline, methylaniline, ethylaniline, butylaniline, biphenyl, methylbiphenyl, and isopropylbiphenyl; an aliphatic hydrocarbon solvent, such as cyclohexane, methylcyclohexane, n-pentane, n-hexane, n-heptane, n-octane, n-nonane, n-decane, tetradecane, decalin, and isopropylcyclohexane; a ketone solvent, such as acetone, methyl ethyl ketone, cyclohexanone, and acetophenone; an ester solvent, such as ethyl acetate, butyl acetate, ethyl cellosolve acetate, methyl benzoate, and phenyl acetate; a polyhydric alcohol and its derivatives, such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol; an alcoholic solvent, such as methanol, ethanol, propanol, isopropanol, and cyclohexanol; a sulfoxide solvent, such as dimethyl sulfoxide; and an amide solvent, such as N-methyl-2-pyrrolidone and N,N-dimethylformamide. These solvents may be used alone or in combination of two or more.

Of the above solvents, in view of solubility, uniform film formation, and viscosity, at least one of toluene, xylene, ethylbenzene, amylbenzene, anisole, 4-methoxytoluene, 2-methoxytoluene, 1,2-dimethoxybenzene, mesitylene, tetrahydronaphthalene, cyclohexylbenzene, 2,3-dihydrobenzofuran, cyclohexanone, methylcyclohexanone is preferably used.

The above solvents and a solvent represented by formula (Si) having a boiling point of 110° C. or higher and a water solubility of 1% by mass or less at 20° C. are preferably used:

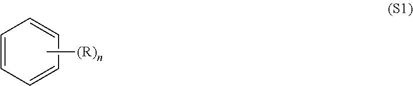

wherein R is independently a substituent having 1 to 20 carbon atoms and n is an integer of 0 to 6.

The film-forming solution (ink composition) preferably comprises the above compound and the solvent represented by formula (Si) having a boiling point of 110° C. or higher and a water solubility of 1% by mass or less at 20° C. The film-forming solution (ink composition) may further comprise, if necessary, a viscosity modifier, a surface tension regulator, a crosslinking initiator, or a crosslinking catalyst, which are preferably selected from those not adversely affecting the device performance even when remaining in the film or those being removable from the film in the film forming process.

In the organic EL device of the invention, each layer other than the organic thin film layer comprising the polymer of the invention may be formed by using a known material. For example, the light emitting layer may be formed from a styrylamine compound, an arylamine compound, or a fluoranthene compound.

Each layer other than the organic thin film layer comprising the polymer of the invention may be formed by a known method, for example, a dry film-forming method such as a vapor deposition method, a sputtering method, a plasma method, and an ion plating method, and a wet film-forming method such as a spin-coating method, a dipping method, and a flow-coating method, and a printing method.

The thickness of each layer is not particularly limited and generally selected from an appropriate range. An excessively large thickness may require a high applied voltage for obtaining a certain level of optical output to reduce the efficiency. An excessively small thickness may cause defects, such as pin holes, and a sufficient luminance is not obtained even when applying an electric field. A thickness of 5 nm to 10 μm is generally appropriate, and more preferably 5 nm to 0.2 um.

The organic EL device may be produced by forming an anode, a light emitting layer, an optional hole injecting/transporting layer, an optional electron injecting/transporting layer, and a cathode by using the materials and the methods for forming the layers. The organic EL device may be produced from a cathode to an anode in the reverse order to the above.

### EXAMPLES

The present invention will be described in more detail. It should be noted that the present invention is not limited to the following examples as long as not departing from the generic concept of the invention.

### NMR Apparatus

The nuclear magnetic resonance spectra of the compounds were measured by GSX-400 (tradename, 400 MHz resolution) manufactured by JEOL Ltd. using heavy chloroform as the solvent.

### GPC Apparatus

The molecular weight was determined by size-exclusion chromatography (SEC) under the following conditions:

Sample solution: a solution of 10 mg of a sample in 10 ml of THF
(tetrahydrofuran);
Injected amount: 100 μl;
Flow rate: 1 ml/min;
Column temperature: 40° C.;
SEC apparatus: HLC-8220 manufactured by Tosoh Corporation;
Detector: refractive index (RI) detector or ultraviolet-visible (UV) light detector;
Column: two TSKgel GMH-XL and one TSKgel G2000-XL manufactured by Tosoh Corporation; and
Standard polystyrene: TSK standard polystyrene manufactured by Tosoh Corporation.

### Synthesis Example 1: Synthesis of 3-[9-(3, 5-dibromophenyl)carbazole-3-yl]-9-phenyl-carbazole (compound I)

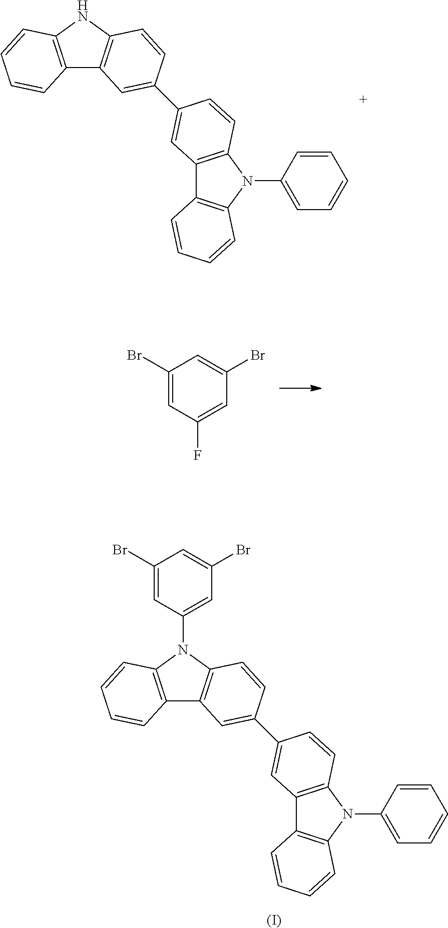

A mixture of 3-(9H-carbazole-3-yl)-9-phenyl-carbazole (16.32 g, 0.04 mol), 1,3-dibromo-5-fluorobenzene (15.23 g, 0.06 mol), cesium carbonate (19.54 g, 0.06 mol), and N-methylpyrrolidone (50 mL) was stirred in a flask at 180° C. for 4 h under heating. The reaction solution was poured into 500 mL of water to precipitate solid. The obtained mixture was neutralized by acetic acid. The solid was collected by filtration and washed with water twice. The obtained solid was dried and recrystallized from toluene twice to obtain 13.6 g of the compound I (53% yield).

### Synthesis Example 2: Synthesis of 9-(3, 5-dibromophenyl)-3, 6-bis(9-phenylcarbazole-3-yl)carbazole (compound II)

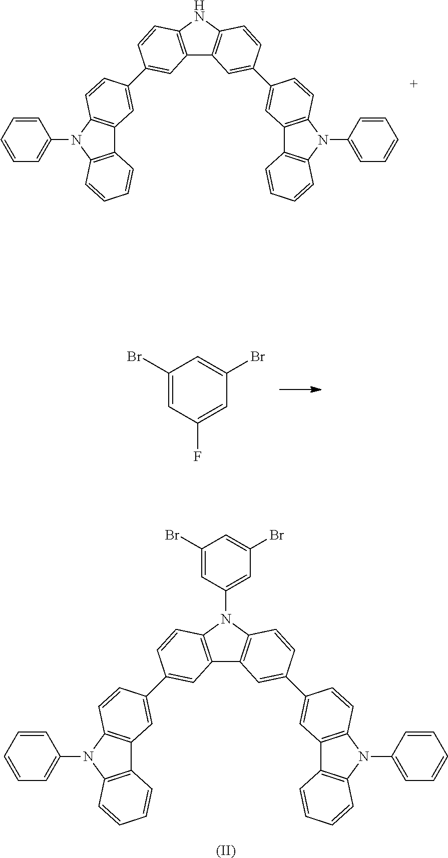 [SMILES: c1ccc(-n2c3ccccc3c3cc(-c4ccc5[nH]c6ccc(-c7ccc8c(c7)c7ccccc7n8-c7ccccc7)cc6c5c4)ccc32)cc1 ; Fc1cc(Br)cc(Br)c1 ; Brc1cc(Br)cc(-n2c3ccc(-c4ccc5c(c4)c4ccccc4n5-c4ccccc4)cc3c3cc(-c4ccc5c(c4)c4ccccc4n5-c4ccccc4)ccc32)c1 ; "(II)"]

A mixture of 3,6-bis(9-phenylcarbazole-3-yl)-911-carbazole (19.5 g, 0.03 mol), 1,3-dibromo-5-fluorobenzene (25 g, 0.1 mol), cesium carbonate (14.7 g, 0.045 mol), and N-methylpyrrolidone (40 ml) was stirred in a flask at 180° C. for 4 h under heating. The reaction solution was poured into 500 ml of water to precipitate solid. The obtained mixture was neutralized by acetic acid. The solid was collected by filtration and washed with water twice. The obtained solid was dried and recrystallized from toluene twice to obtain 17.7 g of the compound II (67% yield).

### Production Example 1: Synthesis of Compound III

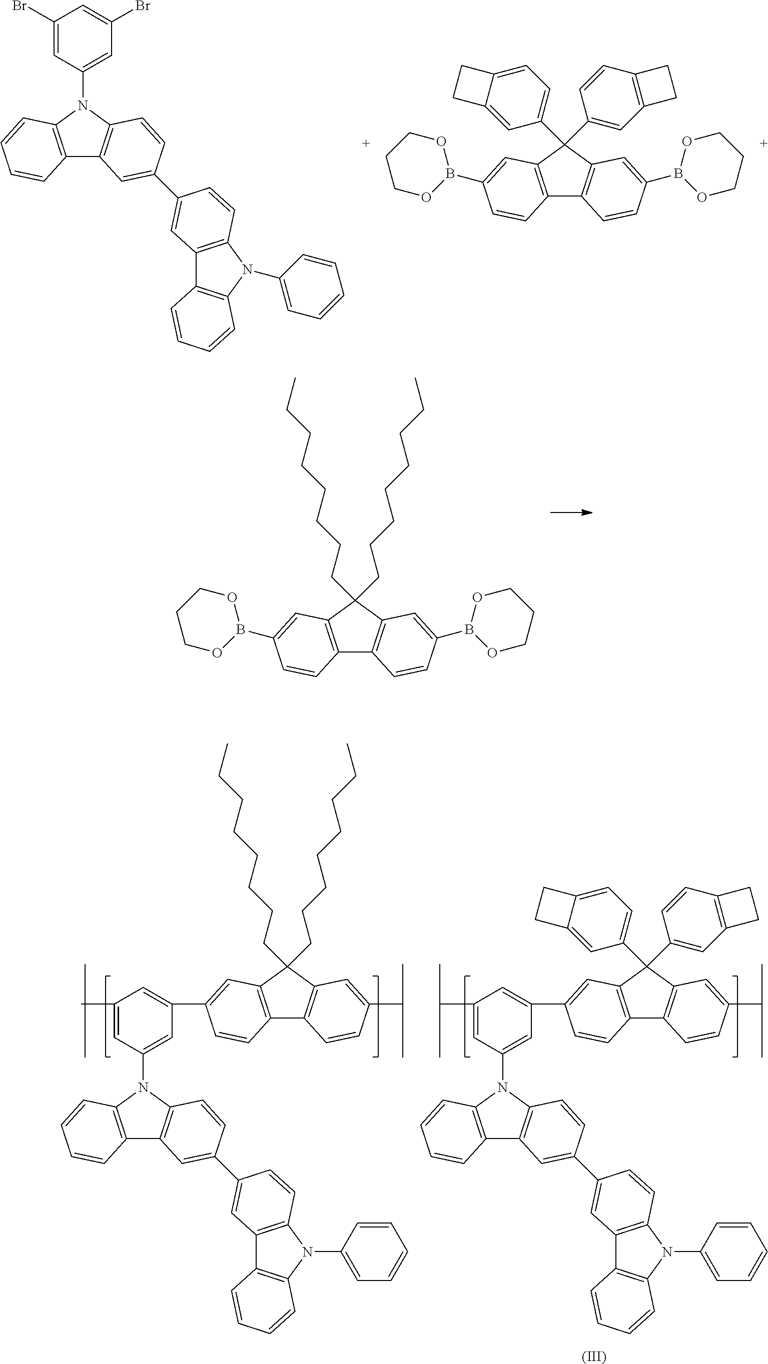

Into a 50-ml flask, 3-[9-(3,5-dibromophenyl)carbazole-3-yl]-9-phenyl-carbazole (1.933 g, 0.003 mol), 2-[7-(1,3,2-dioxaborinane-2-yl)-9,9-dioctyl-fluorene-2-yl]-1,3,2-dioxaborinane (1.346 g, 0.0024 mol), 2,7-bis(1,3,2-dioxaborinane-2-yl)-9,9-bis(4-bicyclo[4.2.0]octa-1,3,5-trienyl)fluorene (0.323 g, 0.0006 mol), tri(o-tolyl)phosphine (18 mg), palladium acetate (7 mg), cesium carbonate (4.92 g), dioxane (9 ml), toluene (6 ml), and water (5 ml) were charged. After repeating the deaeration and the replacement with argon five times, the mixture was stirred at 90° C. for 25 h under heating. After removing the solid in the reaction mixture by filtration, the solution was poured into methanol to precipitate solid. The solid was dissolved in toluene. After adding 5 g of QuadraSil MP (a metal scavenger manufactured by Reaxa Ltd.), the solution was stirred at room temperature. Then, the solid was removed by filtration to obtain a polymer solution. This operation was repeated six times. Then, the polymer solution was reprecipitated in methanol, and the polymer was collected by filtration and dried to obtain 2.4 g of polymer. The GPC measurement using a standard polystyrene showed that Mn was 17,400, Mw was 36,200, and Mw/Mn was 2.07.

### Production Example 2: Synthesis of Compound IV

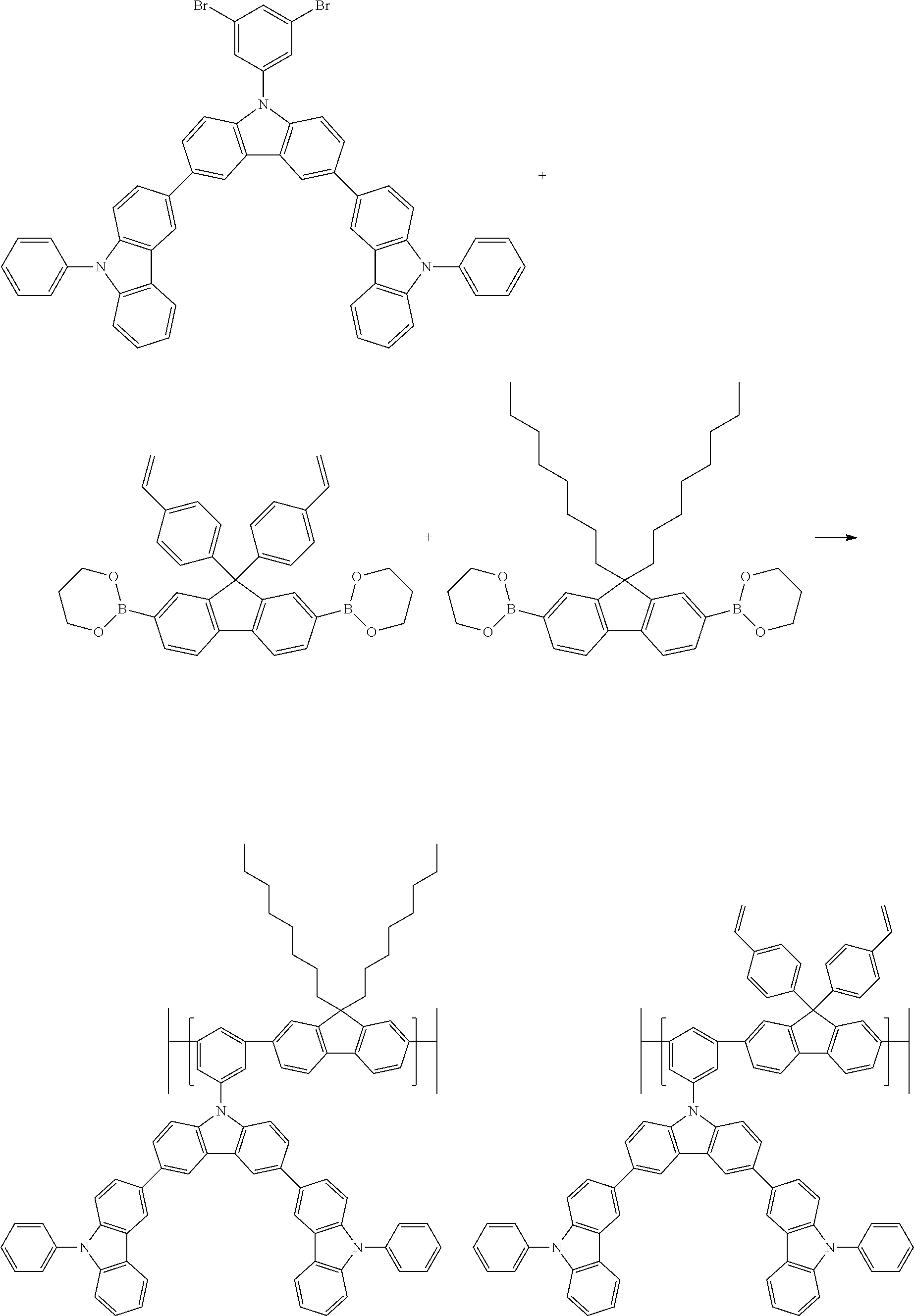

Into a 50-ml flask, 9-(3, 5-dibromophenyl)-3, 6-bis(9-phenylcarbazole-3-yl)carbazole (2.65 g, 0.003 mol), 2-[7-(1,3,2-dioxaborinane-2-yl)-9,9-dioctyl-fluorene-2-yl]-1,3,2-dioxaborinane (1.346 g, 0.0024 mol), 2,7-bis(1,3,2-dioxaborinane-2-yl)-9,9-bis[(4-vinylphenyl)]fluorene (0.323 g, 0.0006 mol), tri(o-tolyl)phosphine (18 mg), palladium acetate (7 mg), cesium carbonate (4.92 g), dioxane (9 ml), toluene (6 ml), and water (5 ml) were charged. After repeating the deaeration and the replacement with argon five times, the mixture was stirred at 90° C. for 25 h under heating. After removing the solid in the reaction mixture by filtration, the solution was poured into methanol to precipitate solid. The solid was dissolved in toluene. After adding 5 g of QuadraSil MP (a metal scavenger manufactured by Reaxa Ltd.), the solution was stirred at room temperature. Then, the solid was removed by filtration to obtain a polymer solution. This operation was repeated six times. Then, the polymer solution was reprecipitated in methanol, and the polymer was collected by filtration and dried to obtain 3.1 g of polymer. The GPC measurement using a standard polystyrene showed that Mn was 11,300, Mw was 56,300, and Mw/Mn was 4.96.

### Production Example 3: Synthesis of Compound V

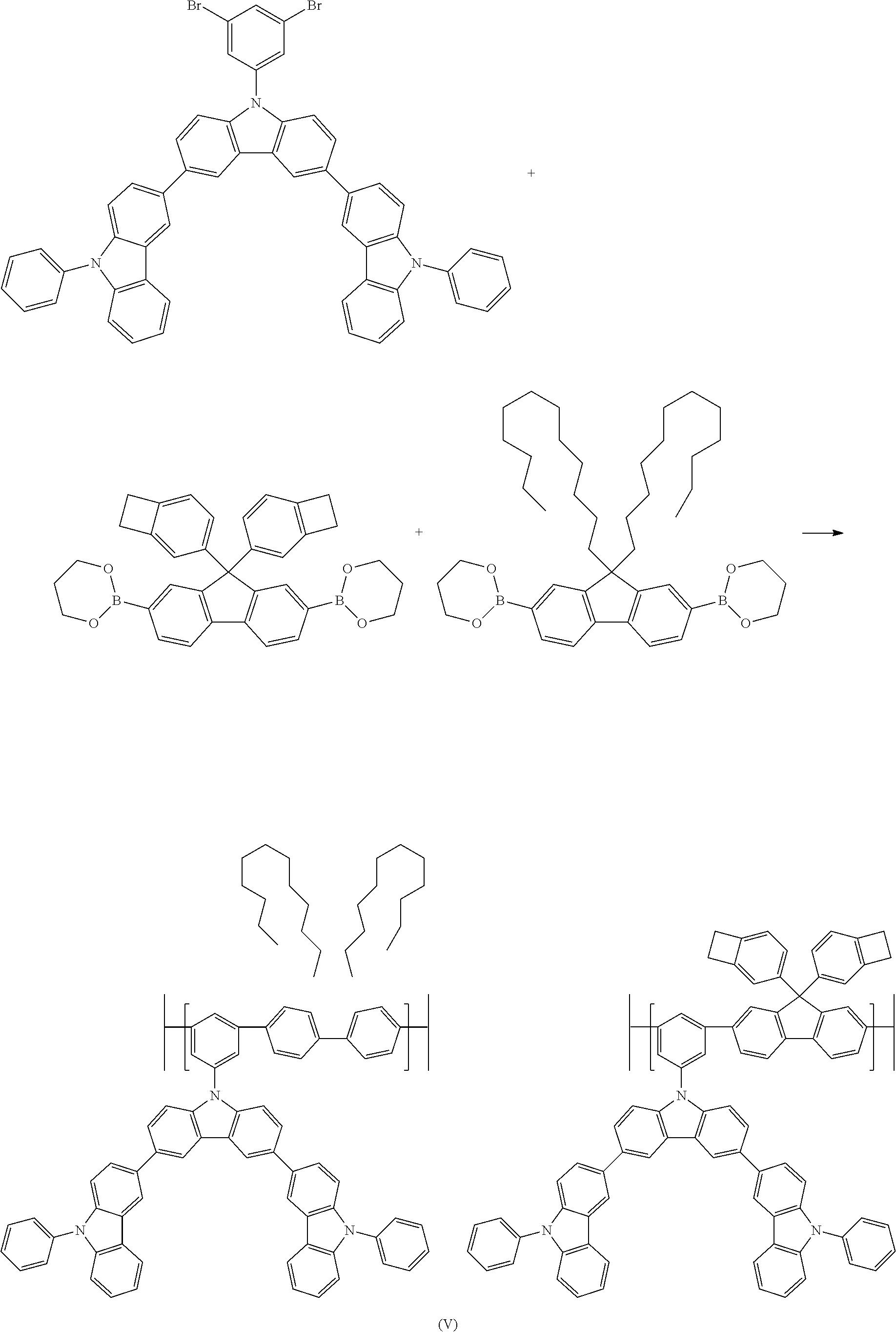

Into a 50-ml flask, 9-(3, 5-dibromophenyl)-3,6-bis(9-phenylcarbazole-3-yl)carbazole (2.65 g, 0.003 mol), 2-[7-(1,3,2-dioxaborinane-2-yl)-9,9-didodecyl-fluorene-2-yl]-1,3,2-dioxaborinane (1.615 g, 0.0024 mol), 2,7-bis (1,3,2-dioxaborinane-2-yl)-9,9-bis(4-bicyclo[4.2.0]octa-1,3,5-trienyl)fluorene (0.323 g, 0.0006 mol), tri(o-tolyl)phosphine (18 mg), palladium acetate (7 mg), cesium carbonate (4.92 g), dioxane (9 nil), toluene (6 ml), and water (5 ml) were charged. After repeating the deaeration and the replacement with argon five times, the mixture was stirred at 90° C. for 25 h under heating. After removing the solid in the reaction mixture by filtration, the solution was poured into methanol to precipitate solid. The solid was dissolved in toluene. After adding 5 g of QuadraSil MP (a metal scavenger manufactured by Reaxa Ltd.), the solution was stirred at room temperature. Then, the solid was removed by filtration to obtain a polymer solution. This operation was repeated six times. Then, the polymer solution was reprecipitated in methanol, and the polymer was collected by filtration and dried to obtain 3.3 g of polymer. The GPC measurement using a standard polystyrene showed that Mn was 11,000, Mw was 23,100, and Mw/Mn was 2.09.

### Production Example 4: Synthesis of Compound VI

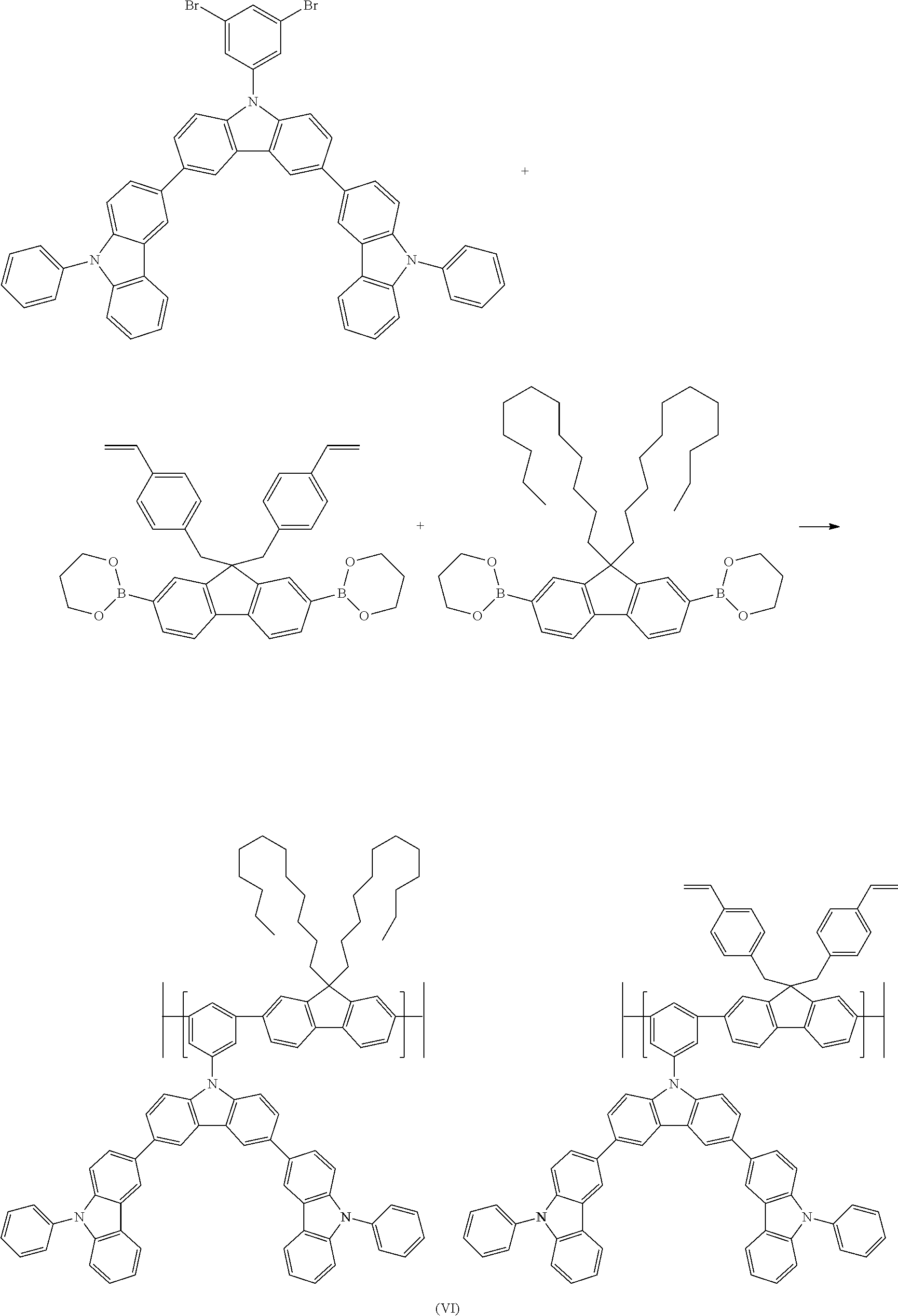

Into a 50-ml flask, 9-(3,5-dibromophenyl)-3,6-bis(9-phenylcarbazole-3-yl)carbazole (2.65 g, 0.003 mol), 2-[7-(1,3,2-dioxaborinane-2-yl)-9,9-didodecyl-fluorene-2-yl]-1,3,2-dioxaborinane (1.615 g, 0.0024 mol), 2,7-bis(1,3,2-dioxaborinane-2-yl)-9,9-bis[(4-vinylphenyl)methyl]fluorene (0.334 g, 0.0006 mol), tri(o-tolyl)phosphine (18 mg), palladium acetate (7 mg), cesium carbonate (4.92 g), dioxane (9 ml), toluene (6 ml), and water (5 ml) were charged. After repeating the deaeration and the replacement with argon five times, the mixture was stirred at 90° C. for 25 h under heating. After removing the solid in the reaction mixture by filtration, the solution was poured into methanol to precipitate solid. The solid was dissolved in toluene. After adding 5 g of QuadraSil MP (a metal scavenger manufactured by Reaxa Ltd.), the solution was stirred at room temperature. Then, the solid was removed by filtration to obtain a polymer solution. This operation was repeated six times. Then, the polymer solution was reprecipitated in methanol, and the polymer was collected by filtration and dried to obtain 3.5 g of polymer. The GPC measurement using a standard polystyrene showed that Mn was 13,000, Mw was 43,100, and Mw/Mn was 3.31.

### Preparation of Solution and Film

A 0.8% by mass solution of the compound of Production Example 1 in toluene (electronics grade manufactured by Kanto Chemical Co., Inc.) was prepared in a glass sample tube (SV-10 manufactured by Nichiden Rika Glass Co., Ltd.). The solution was stirred at 90° C. for 30 min by a stirrer (Laboran Stirrer manufactured by AS ONE Corporation: 10 mm×4 φ) and then cooled for one hour at room temperature to obtain a coating solution. A super white glass (product of Asahi Glass Co., Ltd.) was ultrasonically cleaned in isopropyl alcohol for 5 min and UV-ozone cleaned for 5 min to obtain a glass substrate for coating.

The coating solution was spin-coated on the glass substrate to form a hole transporting layer, thereby obtaining a glass substrate having a hole transporting layer formed thereon. After the spin coating, the substrate was dried on a hot plate at 230° C. for 30 min under heating. The operations from the preparation of the solution to the drying under heating were conducted in a glovebox under nitrogen atmosphere. After the drying under heating, the substrate was cooled to room temperature and the film was partly scraped off. The half of the film thus scraped off was immersed in toluene for 30 s. The shapes around the interfacial portion were measured by New View 6300 manufactured by Zygo Corporation. The degree of insolubilization to toluene was evaluated by the film residue calculated by:

Film residue=thickness of immersed portion/thickness of unimmersed portion.

The compounds of Production Examples 2, 3, and 4 and the comparative compound A were measured for the film residue (insolubilization) in the same manner. The results are shown in Table E-1.

The comparative compound A is a polymer having the following repeating unit, which was produced in accordance with the method described in WO 2014/042265, paragraph [0147]. Mw was 3.5×10⁵ and the molecular weight distribution represented by Mw/Mn was 13.3 when measured by GPC using a standard polystyrene.

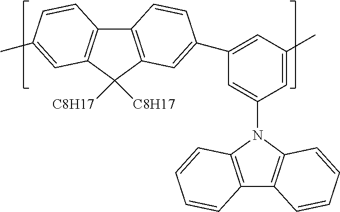

TABLE E-1
Calculation of insolubilization

| | | a | Thickness of toluene-immersed portion (nm) |
|---|---|---|---|
| | Com- | b | Thickness of unimmersed portion (nm) |
| | pound | c | Film reside (insolubilization) (%) |
| Exam- | Production | a | 48.7 |
| ple 1 | example 1 | b | 51.2 |
| | | c | 95.1 |
| Exam- | Production | a | 43.2 |
| ple 2 | example 2 | b | 38.9 |
| | | c | 90.0 |
| Exam- | Production | a | 46.9 |
| ple 3 | example 3 | b | 47.2 |
| | | c | 99.4 |
| Exam- | Production | a | 47.2 |
| ple 4 | example 4 | b | 44.3 |
| | | c | 93.9 |
| Compar- | Compar- | a | 2.2 |
| ative | ative | b | 48.8 |
| exam- | com- | c | 4.5 |
| ple 1 | pound A | | |

### Production of Organic EL Device

### Cleaning of Substrate

A glass substrate of 25 mm×25 mm×1.1 mm thickness having an ITO transparent electrode (product of Geomatec Company) was cleaned by ultrasonic cleaning in isopropyl alcohol for 5 min and then UV ozone cleaning for 5 min.

### Formation of Underlayer

Clevious AI4083 (tradename) manufactured by Heraeus as a material for underlayer was spin-coated on the ITO substrate to form a film with a thickness of 30 nm. Thereafter, unnecessary portion was removed by acetone and then a base substrate was produced by baking in air for 10 min on a hot plate at 200° C. These operations were all conducted in an atmospheric atmosphere.

### Formation of Hole Transporting Layer

A 0.8% by mass solution of the compound of Production Example 1 in toluene (electronics grade manufactured by Kanto Chemical Co., Inc.) was prepared in a glass sample tube (SV-10 manufactured by Nichiden Rika Glass Co., Ltd.). The solution was stirred at 90° C. for 30 min by a stirrer (Laboran Stirrer manufactured by AS ONE Corporation: 10 mm×4 φ) and then cooled for one hour at room temperature to obtain a coating solution. The coating solution was spin-coated on the layer of AI4083 on the base substrate to form a hole transporting layer. After removing unnecessary portion, the substrate thus spin-coated was baked for 30 min on a hot plate at 230° C. to obtain a substrate having a hole transporting layer laminated. The operations from the preparation of the solution and to the baking were all conducted in a glovebox under nitrogen atmosphere.

### Formation of Light Emitting Layer

A 1.6% by weight toluene solution of the compound H-1 (host material) and the compound D-1 (dopant material) was prepared in a mixing ratio of compound H-1: compound D-1=95:5 by mass. The toluene solution was spin-coated on the base substrate having a hole transporting layer laminated into a thickness of 50 nm. Thereafter, unnecessary portion was removed by toluene and then the coated solution was dried under heating on a hot plate at 150° C. 100° C. to obtain a base substrate having a light emitting layer formed thereon. The operations for forming the light emitting layer were all conducted in a glovebox under a nitrogen atmosphere.

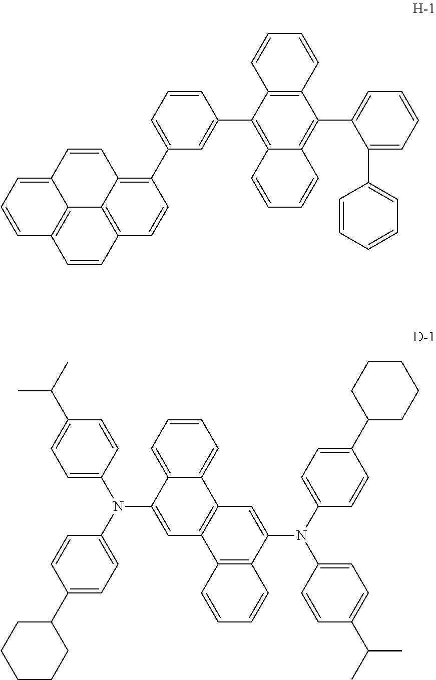

### Vapor Deposition and Sealing

The base substrate having layers thus laminated was conveyed into a vapor deposition chamber and an electron transporting layer was formed by vapor-depositing the compound ET-1 into a thickness of 50 nm. Then, lithium fluoride was vapor-deposited into a thickness of 1 nm and aluminum was vapor-deposited into a thickness of 80 nm. After completing all the vapor deposition processes, the substrate with laminated films was sealed with a bored glass in a glovebox under a nitrogen atmosphere to produce an organic EL device.

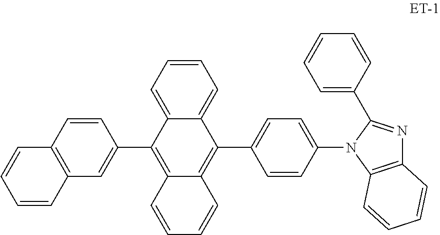 [SMILES: c1ccc(-c2nc3ccccc3n2-c2ccc(-c3c4ccccc4c(-c4ccc5ccccc5c4)c4ccccc34)cc2)cc1]

### Evaluation of Organic EL Device

By driving at a direct current, the obtained organic EL device was allowed to emit light to measure the external quantum efficiency (EQE) at a current density of 10 mA/cm². The devices employing the compounds of Production Examples 2 to 4 and the comparative compound A were also measured for EQE in the same manner. The results are shown in Table E-2.

TABLE E-2
Results of device evaluation

| | Hole transporting layer | EQE (%) |
|---|---|---|
| Example 5 | Production example 1 | 3.3 |
| Example 6 | Production example 2 | 2.9 |
| Example 7 | Production example 3 | 3.4 |
| Example 8 | Production example 4 | 2.8 |

1. An organic electroluminescence device, comprising:
a cathode,
an anode, and
at least one organic thin film layer which is disposed between the cathode and the anode and comprises a light emitting layer,
wherein:
at least one layer of the at least one organic thin film layer is a hole injecting layer or a hole transporting layer; and
at least one of the hole injecting layer and the hole transporting layer comprises a cross-conjugated polymer comprising a structural unit (A) for forming a cross-conjugation system as a repeating unit and a substituent X comprising at least one selected from the group consisting of an arylamine portion, a carbazole portion, and an indole portion as a side chain.

2. The organic electroluminescence device according to claim 1, wherein the structural unit (A) is represented by at least one of formulae (A1) to (A5): 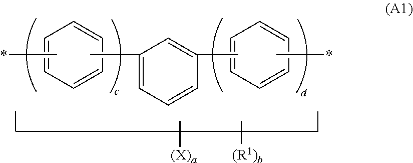
wherein:
X is the substituent X;
each R¹ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
a is 0 or 1;
b is an integer of 0 to 3; and
each of c and d is independently an integer of 0 to 3; 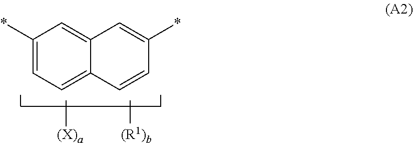
wherein:
X is the substituent X;
each R¹ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
a is 0 or 1; and
b is an integer of 0 to 3; 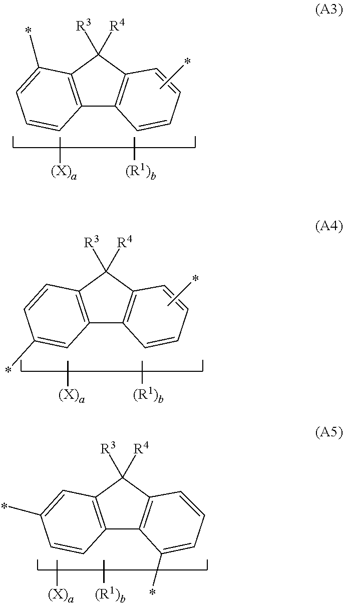
wherein:
X is the substituent X;
each R¹ is independently a tertiary alkyl group comprising 4 to 60 carbon atoms, an aryl group, a mono valent aromatic heterocyclic group, an aralkyl group, or a substituted amino group;
each of R³ and R⁴ is independently a hydrogen atom, an alkyl group comprising 1 to 60 carbon atoms, an aryl group, a mono valent aromatic heterocyclic group, an aralkyl group, or a substituted amino group;
* is a bonding site in a main skeleton;
a is 0 or 1; and
b is an integer of 0 to 3.

3. The organic electroluminescence device according to claim 1, wherein the substituent X is represented by formula (HTG1): 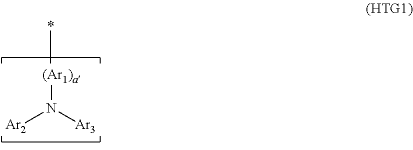
wherein:
* is a bonding site;
each of Ar₁, Ar₂, and Ar₃ is independently a substituted or unsubstituted aromatic hydrocarbon group comprising 6 to 60 ring carbon atoms, a substituted or unsubstituted aromatic heterocyclic group comprising 5 to 60 ring atoms, or a substituted or unsubstituted arylamino group;
Ar₂ and Ar₃ are optionally bonded to each other to form a ring;
a′ is 0 or 1;
when a′ is 0, the bonding site * exists on the nitrogen atom; and
when a′ is 1, the bonding site * exists on Ar₁, Ar₂, or Ar₃.

4. The organic electroluminescence device according to claim 1, wherein the substituent X is bonded to the structural unit (A).

5. The organic electroluminescence device according to claim 1, wherein the cross-conjugated polymer further comprises a structural unit (B) comprising at least one selected from the group consisting of an aromatic hydrocarbon ring group and an aromatic heterocyclic group as a repeating unit.

6. The organic electroluminescence device according to claim 5, wherein the structural unit (B) is represented by at least one of formula (B1) to (B3): 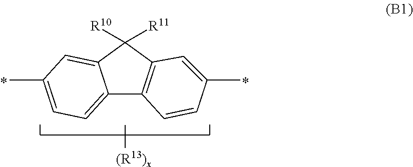
wherein:
each of R¹⁰ and R¹¹ is independently a hydrogen atom, an alkyl group comprising 1 to 60 carbon atoms, an aryl group, a mono valent aromatic heterocyclic group, an aralkyl group, or a substituted amino group;
each R¹³ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton; and
x is an integer of 0 to 3; 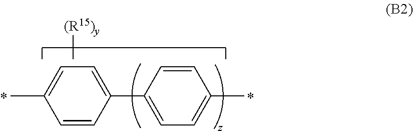
wherein:
each R¹⁵ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
y is an integer of 0 to 3; and
z is 0 or 1; 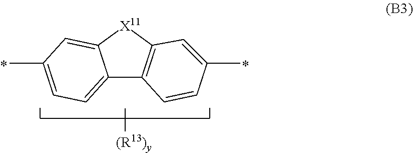
wherein:
X¹¹ is an oxygen atom or a sulfur atom;
each R¹³ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton; and
y is an integer of 0 to 3.

7. The organic electroluminescence device according to claim 5, wherein a molar ratio M_{A} of the structural unit (A) and a molar ratio M_{B} of the structural unit (B) satisfy a relationship (1):
M_{A}>M_{B} (1).

8. The organic electroluminescence device according to claim 7, wherein M_{A} and M_{B} satisfy a relationship (2):
M_{A}>M_{B} (2).

9. The organic electroluminescence device according to claim 1, wherein the cross-conjugated polymer further comprises a structural unit (C) comprising a cross-linkable group as a repeating unit.

10. The organic electroluminescence device according to claim 9, wherein the structural unit (C) is represented by at least one of formulae (C1) and (C2): 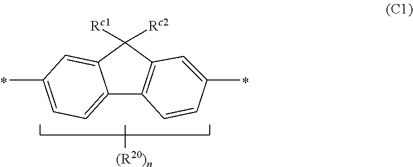
wherein:
each of R^{c1} and R^{c2} is independently a group comprising a small ring comprising 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;
each R²⁰ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton; and
n is an integer of 0 to 3; 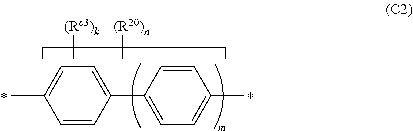
wherein:
each R^{c3} is a group comprising a small ring comprising 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, a group comprising a silanol structure;
each R²⁰ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
m is 0 or 1;
n is an integer of 0 to 3; and
k is an integer of 1 to 3.

11. The organic electroluminescence device according to claim 1, wherein a weight average molecular weight of the cross-conjugated polymer is 50,000 to 1,500,000.

12. A cross-conjugated polymer, comprising:
a structural unit (A) and a structural unit (B) as repeating units and
a substituent X as a side chain,
wherein:
the structural unit (A) forms a cross-conjugation system;
the structural unit (B) comprises at least one of an aromatic hydrocarbon ring group and an aromatic heterocyclic group in a main chain;
the substituent X comprises at least one of an arylamine portion, a carbazole portion, and an indole portion; and
a molar ratio M_{A} of the structural unit (A) and a molar ratio M_{B} of the structural unit (B) satisfy a relationship (1):
M_{A}≥M_{B} (1).

13. The cross-conjugated polymer according to claim 12, wherein M_{A} and M_{B} satisfy a relationship (2):
M_{A}>M_{B} (2).

14. The cross-conjugated polymer according to claim 12, wherein the structural unit (A) is represented by at least one of formulae (A1) to (A5): 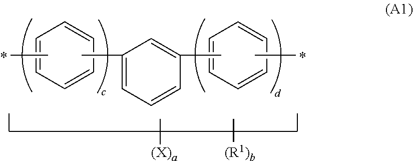
wherein:
X is the substituent X;
each R¹ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
a is 0 or 1;
b is an integer of 0 to 3; and
each of c and d is independently an integer of 0 to 3; 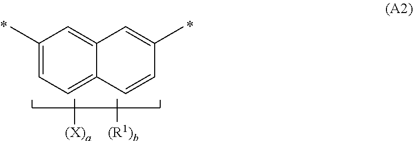
wherein:
X is the substituent X;
each R¹ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
a is 0 or 1; and
b is an integer of 0 to 3; 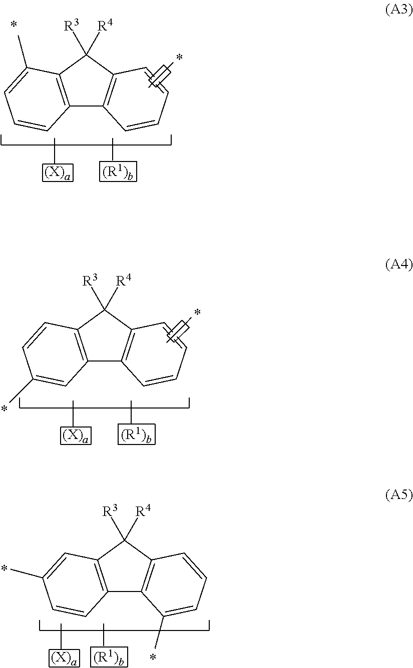
wherein:
X is the substituent X;
each R¹ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
each of R³ and R⁴ is independently a hydrogen atom, an alkyl group comprising 1 to 60 carbon atoms, an aryl group, a mono valent aromatic heterocyclic group, an aralkyl group, or a substituted amino group;
* is a bonding site in a main skeleton;
a is 0 or 1; and
b is an integer of 0 to 3.

15. The cross-conjugated polymer according to claim 12, wherein the substituent X is represented by formula (HTG1): 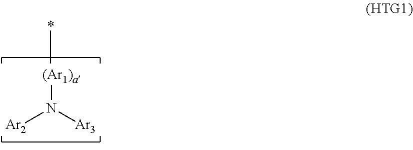
wherein:
* is a bonding site;
each of Ar₁, Ar₂, and Ar₃ is independently a substituted or unsubstituted aromatic hydrocarbon group comprising 6 to 60 ring carbon atoms, a substituted or unsubstituted aromatic heterocyclic group comprising 5 to 60 ring atoms, or a substituted or unsubstituted arylamino group;
Ar₂ and Ar₃ are optionally bonded to each other to form a ring;
a′ is 0 or 1;
when a′ is 0, the bonding site * exists on the nitrogen atom; and
when a′ is 1, the bonding site * exists on Ar₁, Ar₂, or Ar₃.

16. The cross-conjugated polymer according to claim 12, wherein the substituent X is bonded to the structural unit (A).

17. The cross-conjugated polymer according to claim 12, wherein the structural unit (B) is represented by at least one of formula (B1) to (B3): 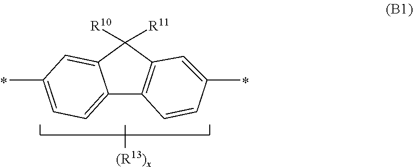
wherein:
each of R¹⁰ and R¹¹ is independently a hydrogen atom, an alkyl group comprising 1 to 60 carbon atoms, an aryl group, a mono valent aromatic heterocyclic group, an aralkyl group, or a substituted amino group;
each R¹³ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main chain; and
x is an integer of 0 to 3; 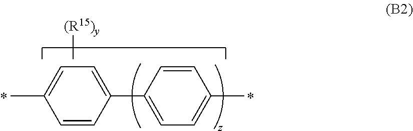
wherein:
each R¹⁵ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
y is an integer of 0 to 3; and
z is 0 or 1; 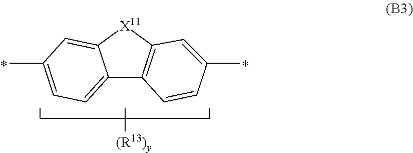
wherein:
X¹¹ is an oxygen atom or a sulfur atom;
each R¹³ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton; and
y is an integer of 0 to 3.

18. The cross-conjugated polymer according to claim 12, wherein the cross-conjugated polymer further comprises a structural unit (C) comprising a cross-linkable group as a repeating unit.

19. The cross-conjugated polymer according to claim 18, wherein the structural unit (C) is represented by at least one of formulae (C1) and (C2): 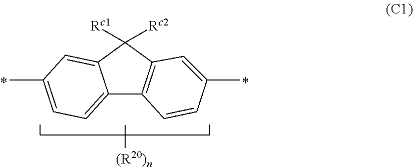
wherein:
each of R^{c1} and R^{c2} is independently a group comprising a small ring comprising 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;
each R²⁰ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton; and
n is an integer of 0 to 3; 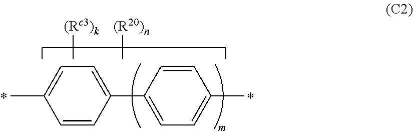
wherein:
each R^{c3} is a group comprising a small ring comprising 3 to 4 ring atoms, a vinyl group, an ethynyl group, a butenyl group, a group comprising an acryl structure, a group comprising an acrylate structure, a group comprising an acrylamide structure, a group comprising a methacryl structure, a group comprising a methacrylate structure, a group comprising a methacrylamide structure, a group comprising a vinyl ether structure, a vinylamino group, or a group comprising a silanol structure;
each R²⁰ is independently a tertiary alkyl group comprising 4 to 60 carbon atoms, an aryl group, a mono valent aromatic heterocyclic group, an alkoxy group comprising 1 to 20 carbon atoms, a cycloalkoxy group comprising 3 to 10 ring carbon atoms, an aryloxy group comprising 6 to 30 ring carbon atoms, an aralkyl group, a substituted amino group, a substituted silyl group, a cyano group, or a halogen atom;
* is a bonding site in a main skeleton;
m is 0 or 1;
n is an integer of 0 to 3; and
k is an integer of 1 to 3.

20. The cross-conjugated polymer according to claim 18, wherein a molar ratio M_{A} of the structural unit (A), a molar ratio M_{B} of the structural unit (B), and a molar ratio M_{C} of the structural unit (C) satisfy a relationship (3):
M_{A}≥Mₛ+M_{C} (3).

21. The cross-conjugated polymer according to claim 12, wherein a weight average molecular weight of the cross-conjugated polymer is 50,000 to 1,500,000.

22. A material for electronic device, comprising the polymer according to claim 12.

23. A material for organic electroluminescence device, comprising the polymer according to claim 12.

24. An organic electroluminescence device, comprising
an organic thin film layer between an anode and a cathode,
wherein:
the organic thin film layer comprises one or more layers which comprise a light emitting layer; and
at least one layer of the organic thin film layer comprises the polymer according to claim 12.

25. The organic electroluminescence device according to claim 24, wherein the organic thin film layer comprises a hole transporting layer, and the hole transporting layer comprises the polymer.

26. A coating solution, comprising:
the polymer according to claim 12 and
a solvent.

27. A method of producing an organic electroluminescence device, the method comprising:
forming a thin film layer by using the coating solution according to claim 26.

28. The method according to claim 27, wherein the thin film layer is formed by a wet film-forming method.
